(19) United States
(12) Patent Application Publication
(10) Pub. No.: US 2015/0067923 A1
(43) Pub. Date: Mar. 5, 2015

(54) Transgenic Plants And A Transient Transformation System For Genome-Wide Transcription Factor Target Discovery

(71) Applicant: New York University, New York, NY (US)

(72) Inventors: Gloria Coruzzi, New York, NY (US); Kenneth Birnbaum, Brookly, NY (US); Bastiaan Bargmann, San Diego, CA (US); Gabriel Krouk, Grabels (FR)

(73) Assignee: New York University, New York, NY (US)

(21) Appl. No.: 14/457,402

(22) Filed: Aug. 12, 2014

Related U.S. Application Data
(60) Provisional application No. 62/011,729, filed on Jun. 13, 2014.
(60) Provisional application No. 61/865,438, filed on Aug. 13, 2013.

(51) Int. Cl. C12Q 1/68; C12N 15/82

(52) U.S. Cl. CPC C12Q 1/6806; C12N 15/8241
USPC 800/298; 435/412; 435/414; 435/415; 435/416; 435/411; 435/417; 435/419; 435/6.12; 506/3; 536/23.4

(57) ABSTRACT

Plant genes regulated by transcription factors that control the gene network response to an environmental perturbation or signal are described. This class of genes responds to the perturbation of a transcription factor and the signal it transduces, but surprisingly, without stable binding of the transcription factor. These genes represent members of the “dark matter” of metabolic regulatory circuits.

The invention involves the transgenic manipulation of these “response genes” and/or the genes encoding their regulatory transcription factors in plants so that their respective gene products are either overexpressed or underexpressed in the plant in order to confer a desired phenotype.

The invention also relates to a rapid technique named “TARGET” (Transient Assay Reporting Genome-wide Effects of Transcription factors) for determining such “response genes” and their transcription factors by perturbation of the expression of the transcription factors of interest in protoplasts of any plant species.

This application claims the benefit of U.S. Provisional Application No. 61/865,438 filed on Aug. 13, 2013 and U.S. Provisional Application 62/011,729 filed on Jun. 13, 2014, the entire contents of both of which are incorporated by reference herein in their entireties.

TABLE OF CONTENTS

| | | | Page |
|---|---|---|---|
| 1. | INTRODUCTION | | 1 |
| 2. | BACKGROUND | | 1 |
| 3. | SUMMARY | | 2 |
| | 3.1. | TERMINOLOGY | 16 |
| 4. | DESCRIPTION OF THE FIGURES | | 33 |
| 5. | DETAILED DESCRIPTION | | 42 |
| | 5.1. | Response genes and transcription factors | 47 |
| | 5.2. | Transgenic Plants | 54 |

| | | |
|---|---|---|
| 5.2.1. | Modulation of Gene Expression | 54 |
| 5.2.2. | Transfection | 55 |
| 5.2.3. | Nucleic Acid Constructs | 61 |
| 5.2.4. | Host Cells | 65 |
| 5.2.5. | Promoters and Other Regulatory Sequences | 67 |
| 5.2.6. | Isolating Related Promoter Sequences | 73 |
| 5.2.7. | Cell-Type Preferential Transcription | 76 |
| 5.2.8. | Selection and Identification of Transfected | 76 |
| | Host Cells | |
| 5.2.9. | Plant Regeneration | 78 |
| 5.2.10. | Plants | 81 |
| 5.2.11. | Cultivation | 83 |
| 5.2.12. | Products of Transgenic Plants | 84 |

| | | |
|---|---|---|
| 5.3. | Components of The TARGET system | 87 |

| | | |
|---|---|---|
| 5.3.1. | Transcription Factors | 87 |
| 5.3.2. | Localization Signals and Inducing Agents | 87 |
| 5.3.3. | Expression System and Selectable Markers | 87 |
| 5.3.4. | Detecting the Level of mRNA Expressed in | 89 |
| | Host Cells | |
| 5.3.5. | Detecting TF Binding to Gene Targets | 90 |
| 5.3.6. | Identifying Conserved Connections Across | 91 |
| | Species | |

| 6 | EXAMPLE 1 | | 91 |
|---|---|---|---|
| | 6.1. | Introduction | 91 |
| | 6.2. | Materials and Methods | 92 |
| | 6.3. | Results | 95 |
| | 6.4. | Discussion | 96 |
| 7 | EXAMPLE 2 | | 98 |
| | 7.1. | Introduction | 98 |
| | 72 | Materials and Methods | 98 |
| | 7.3. | Results | 102 |
| | 7.4. | Discussion and Concluding Remarks | 113 |
| 8. | EXAMPLE 3 | | 116 |
| | 8.1. | Plant Growth and Treatment | 116 |
| | 8.2. | Microarray Experiments and Analysis | 117 |
| | 8.3. | Network Analysis | 117 |
| 9. | EXAMPLE 4 | | 121 |
| | 9.1. | Building Crop Networks | 121 |
| 10. | EXAMPLE 5 | | 123 |
| | 10.1. | Introduction | 123 |
| | 10.2. | Materials and Methods | 125 |
| | 10.3. | Results | 130 |
| | 10.4. | Discussion | 244 |

| | | |
|---|---|---|
| 11. | EXAMPLE 6 | 247 |
| 12. | EXAMPLE 7 | 253 |
| 13. | EQUIVALENTS | 259 |

### 1. INTRODUCTION

This invention relates to plant genes regulated by transcription factors that control the gene network response to an environmental perturbation or signal, and the manipulation of the expression of these “response genes” and/or their regulatory transcription factors in transgenic plants to confer a desired phenotype. The invention also relates to a rapid technique named “TARGET” (Transient Assay Reporting Genome-wide Effects of Transcription factors) for determining such “response genes” and their regulatory transcription factors as well as the structure of the involved gene regulatory networks (GRN)— including “transient” targets of transcription factors (TF)— by transiently perturbing the expression of the transcription factors of interest and the signals they transduce in protoplasts of any plant species.

### 2. BACKGROUND

Determining the fundamental structure of gene regulatory networks (GRN) is a major challenge of systems biology. In particular, inferring GRN structure from comprehensive gene expression and transcription factor (TF)-promoter interaction datasets has become an increasingly sought after aim in both fundamental and agronomical research in plant biology (Bonneau et al, 2007, Cell 131:1354-1365; Ruffel et al., 2010, Plant Physiol 152:445-452). A crucial step for the assessment of GRN is the identification of the direct TF-target genes.

Transgenic plant lines expressing tagged versions of the TF-of-interest can be used together with transcriptomic and DNA-binding analyses to obtain high-confidence lists of direct targets (see e.g., Mönke et al., 2012, Nucleic acids research 40:8240-825). However, the generation of such transgenics can be a limiting factor, especially in large-scale studies or in non-model species.

Another major challenge in systems biology is the generation of gene regulatory networks (GRNs) that describe, and ideally, predict how the network will respond to perturbation. Currently, the global structure of a GRN is modeled by inferring regulatory relationships between transcription factors (TFs) and their target genes from genomic data (Krouk et al., 2010, Genome Biology 11:R123; Brady et al., 2011, Molecular Systems Biology 7:459; Petricka et al., 2011, Trends in Cell Biology 21:442). While diverse experimental approaches have been devised to validate interactions between specific TFs and their targets (Matallana-Ramirez et al., 2013, Molecular Plant [epub ahead of print, doi: 10.1093/mp/sst012]; Bargmann et al., 2013, Molecular Plant 6(3):978; Gorte et al., 2011, Plant Transcription Factors, vol. 754, pp. 119-141; Iwata et al., 2011, Plant Transcription Factors, vol. 754, pp. 107-117; Wehner et al., 2011, Frontiers in Plant Science 2:68), the “gold standard” in the field has been to identify primary TF-targets as genes that are both transcriptionally regulated and whose promoter region is bound by the TF of interest (Oh et al., 2009, The Plant Cell Online 21:403). However, a GRN built purely on this “gold standard” rule (Reeves et al., 2011, Plant Molecular Biology 75:347; Gorski et al., 2011, Nucleic Acids Research 39:9536; Hull et al., 2013, BMC Genomics 14:92; Fujisawa et al., 2011, Planta 235:1107), renders a static network that only includes targets stably bound by a TF under the studied conditions, and likely underestimates the dynamic interactions occurring in vivo.

For example, in higher plants, fluctuating nitrogen levels in the soil cause rapid and dramatic changes in plant gene expression. Nitrogen is both a metabolic nutrient and signal that broadly and rapidly reprograms genome-wide responses. While genomic responses to nitrogen have been studied for many years, only a small number of genes in nitrogen genome-wide reprogramming have been identified. The unidentified genes represent the so-called “dark matter” of such metabolic regulatory circuits, a crucial problem in understanding system-wide genetic regulation in many fields.

### 3. SUMMARY

Plant genes regulated by transcription factors that control the gene network response to an environmental perturbation or signal (e.g., nitrogen, water, sunlight, oxygen, temperature) are described. These genes respond rapidly to their environment, but surprisingly, there is no evidence of direct transcription factor interaction. More particularly, the large class of genes described herein (and exemplified in Tables 1, 2, 19, 20, and 23) respond to the perturbation of a regulatory transcription factor and the signal it transduces, but in fact are not stably bound to the transcription factor, and yet are most relevant to the signal induced in vivo—in other words, they represent members of the “dark matter” of metabolic regulatory circuits. The invention involves the transgenic manipulation of these “response genes” and/or the genes encoding their regulatory transcription factors in plants so that their respective gene products are either overexpressed or underexpressed in the plant in order to confer a desired phenotype; e.g., increased N usage (to enhance plant growth/biomass) or N storage/yield (to enhance N storage and/or protein accumulation in seeds of seed crops).

The invention is based, in part, on the development of a rapid technique named “TARGET” (Transient Assay Reporting Genome-wide Effects of Transcription factors) that uses transient transformation of a plasmid containing a glucocorticoid receptor (GR)-tagged TF in protoplasts to study the genome-wide effects of TF activation. The TARGET system can be used to rapidly retrieve information on direct TF target genes in less than two week's time. The technique can be used as a part of various experimental designs, as show in FIG. 1. The core of the technique makes use of an isolated nucleic acid molecule encoding a chimeric protein comprising a transcription factor fused to a domain comprising an inducible cellular localization signal and an independently expressed selectable marker. A host cell such as a plant protoplast may then be transiently transfected with the nucleic acid molecule. The selectable marker allows for the determination of which cells have been successfully transfected. The TF-inducible signal fusion is sequestered in one cellular location until this retention mechanism is released through treatment with a localization-inducing signal, such as a small molecule. To determine the transcription factor response in the presence of an environmental signal, pre-treatment with such a signal may optionally be performed before the treatment with the cellular localization-inducing signal. mRNA transcripts may then be measured by microarray analysis or other suitable method in those cells identified to be successfully transfected by means of the selectable marker. To distinguish between primary and secondary response genes, a translation inhibitor such as cyclohexamide may optionally be used to inhibit translation of mRNA. Likewise, to determine the binding properties of the transcription factors to their target sequences, an additional step of ChIP-Seq analysis may be optionally added concurrently to microarray analysis which detects mRNAs of TF targets. ChIP-Seq analysis may be done on the same cell samples as the microarray analysis.

While not intending to be bound to any theory of operation, using the TARGET system, gene networks have been identified that are regulated by TFs via transient associations with the target gene. Unexpectedly, these transient TF targets were found to be biologically relevant in controlling responsiveness to the applied signal/pertubation/cue. The target genes of interest are referred to herein as “response genes” that are regulated by what is referred to herein as their transiently associated “touch and go” or “hit and run” transcription factors. Conventional wisdom has focused on the “Golden Set” of genes stably bound and regulated by a TF, and has failed to uncover these transient associations described herein.

As a proof-of-principle candidate, the well-studied transcription factor, Abscicic acid insensitive 3 (ABI3) was investigated using TARGET, as described in more detail herein in Section 6 (Example 1). The de novo identification of the abscisic acid response element (ABRE) and a majority of the previously classified direct targets was established by use of the TARGET method, confirming its applicability. The TARGET system was then further modified, as described in further detail in Sections 7 and 10 (Examples 2 and 5), to identify genes transiently bound and regulated by the TF of the system in response to an environmental signal. These modifications allowed for the discovery of a “hit-and-run” (“touch-and-go”) mode-of-action for a proof-of-principle transcription factor candidate, bZIP1, where bZIP1 “hits” its target, initiates transcription, then dissociates (“run”), leaving the transcription going on even without bZIP1 binding to the promoter. As evidence that transcription of a gene initiated by “the Hit” continues after “the Run,” an affinity-tagged UTP was used to label and capture newly synthesized mRNA, as described in Section 11 (Example 6). By adding this UTP affinity label at a time-point when bZIP1 is not detectably bound, it was determined that response genes were still actively transcribed. Section 12 (Example 7) describes the discovery that the transient TF-targets detected specifically in the TARGET cell-based system make a unique contribution to understanding how signal transduction occurs in planta, while eluding detection in planta.

In Section 8 (Example 3), a method for identifying nitrogen-regulated connections conserved across model species and crops is detailed. This method is a rapid way to assess whether the function of a gene of interest is conserved across species and enables the enhancement of the translational discoveries of the TARGET system. The method of Section 8 may be used as an alternative or supplement to using the TARGET system directly in protoplasts of crops or other plant species. Section 9 (Example 4) also describes a method for identifying networks conserved across species to identify translational targets that may be used as an alternative or supplement to the TARGET system.

One advantage of the TARGET system is the ability to study gene regulatory networks and targets of transcription factors in a transient assay system, which means the method can be applied to plants that cannot be stably transformed. Protoplasts can be made from any plant species, and a transcription factor of interest can be transiently expressed to identify its targets genome-wide. Target genes of transcription factors can be rapidly identified because the method does not rely on the use of transgenic plants, which normally have to be stably transformed. Also, the TARGET technique allows for cross-species studies in order to analyze evolutionary conserved networks using genes from a poorly characterized plant genus or species in a better characterized model genus, such as Arabidopsis, which has a fully sequenced genome and has microarray chip data available. This also has important implications for translational studies of gene function, from data-rich models (e.g. Arabidopsis) to data-poor crops. By providing the ability to do reciprocal cross species genetic network comparisons, the TARGET technique allows for the determination of TF-target connections that are evolutionarily conserved and therefore likely the most important elements of transcription factor networks. The optional modifications to the TARGET system confers the further advantage of the ability to detect gene networks that are controlled transiently in response to environmental signals by TF interactions that have been previously ignored. TF regulation is not always associated with stable TF binding. The TARGET system uncovers TF targets that would otherwise be missed in other systems that require TF binding to identify gene targets. The TARGET system allows for the identification of the functional mode of action for any TF within and across species.

The most recent advance in the field of nitrogen-signaling uncovered a master transcription factor, NLP7, which when mutated, affects >58% of the nitrogen-responsive genes in plants, yet can be shown to bind to only 10% of these targets. This conundrum represents a general problem in the field of transcription, and a particular problem in metabolic signaling, where TF binding is a poor indicator of system-wide gene regulation. In fact, most GRN studies have focused on determining when and how TF binding does, or does not, result in activation of its target genes. Such TF-binding approaches have missed the “dark matter” of signal transduction. The TARGET system has revealed that the largest class of genes responding to the perturbation of a TF and a signal it transduces are in fact not stably bound to the TF, and this class of genes which has the most relevance to the signal transduced has been missed in all TF studies to date. Several unique aspects of the system described enable the discovery of this large set of primary TF targets that are regulated by, but do not stably bind to the TF.

In one embodiment, the present invention is directed to a transgenic plant that ectopically expresses one or more touch and go (hit and run) transcription factor genes and exhibits a desired phenotype, wherein the said one or more genes comprises a polynucleotide that encodes At1g01060, At1g01720, At1g13300, At1g15100, At1g22070, At1g25550, At1g25560, At1g29160, At1g43160, At1g51700, At1g51950, At1g53910, At1g66140, At1g68670, At1g68840, At1g74660, At1g74840, At1g75390, At1g77450, At1g80840, At2g04880, At2g20570, At2g22430, At2g22850, At2g24570, At2g25000, At2g28510, At2g28550, At2g30250, At2g33710, At2g38470, At2g46830, At3g01560, At3g04070, At3g06590, At3g20770, At3g25790, At3g46130, At3g47620, At3g51920, At3g54620, At3g60490, At3g61150, At3g61890, At3g62420, At4g17490, At4g17500, At4g24240, At4g27410, At4g31800, At4g34590, At4g36540, At4g37180, At4g37260, At4g37610, At4g37730, At5g05410, At5g06800, At5G10030, At5g13080, At5g14540, At5g24800, At5g39610, At5g44190, At5g47230, At5g48655, At5g49450, At5g49520, At5g56270, At5g60850, At5g63790, At5G65210, or At5g65640. In another embodiment, the present invention is directed to a transgenic plant that ectopically expresses one or more touch and go (hit and run) transcription factor genes and exhibits a desired phenotype, wherein the said one or more genes comprises a polynucleotide that encodes At1g01060, At1g01720, At1g13300, At1g15100, At1g25550, At1g25560, At1g29160, At1g51700, At1g51950, At1g53910, At1g66140, At1g68670, At1g68840, At1g74660, At1g75390, At1g77450, At1g80840, At2g04880, At2g22850, At2g24570, At2g28510, At2g28550, At2g30250, At2g33710, At3g04070, At3g06590, At3g20770, At3g25790, At3g46130, At3g47620, At3g51920, At3g54620, At3g60490, At3g62420, At4g17490, At4g24240, At4g27410, At4g31800, At4g34590, At4g36540, At4g37180, At4g37610, At4g37730, At5g05410, At5g06800, At5G10030, At5g13080, At5g39610, At5g47230, At5g49520, At5g56270, At5g60850, At5g63790, At5G65210, or At5g65640.

In one embodiment, the present invention is directed to an isolated nucleic acid molecule that encodes (a) a chimeric protein comprising a transcription factor fused to a domain comprising an inducible nuclear localization signal; and (b) an independently expressed selectable marker. In another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the nucleic acid molecule is a DNA plasmid. In yet another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the domain comprising an inducible nuclear localization signal is glucocorticoid receptor. In yet another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the nucleic acid molecule is a DNA plasmid and the domain comprising an inducible nuclear localization signal is glucocorticoid receptor.

In one embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the selectable marker is a fluorescent selection marker. In another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the nucleic acid molecule is a DNA plasmid, and wherein the selectable marker is a fluorescent selection marker. In yet another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the domain comprising an inducible nuclear localization signal is glucocorticoid receptor. In yet another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the nucleic acid molecule is a DNA plasmid, the domain comprising an inducible nuclear localization signal is glucocorticoid receptor, and the selectable marker is a fluorescent selection marker.

In one embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the selectable marker is green fluorescent protein, yellow fluorescent protein, red fluorescent protein, cyan fluorescent protein, or blue fluorescent protein. In another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the nucleic acid molecule is a DNA plasmid, and wherein the selectable marker is a green fluorescent protein, yellow fluorescent protein, red fluorescent protein, cyan fluorescent protein, or blue fluorescent protein. In yet another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the domain comprising an inducible nuclear localization signal is glucocorticoid receptor. In yet another embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the nucleic acid molecule is a DNA plasmid, the domain comprising an inducible nuclear localization signal is glucocorticoid receptor, and the selectable marker is green fluorescent protein, yellow fluorescent protein, red fluorescent protein, cyan fluorescent protein, or blue fluorescent protein.

In one embodiment, the present invention is directed to an isolated nucleic acid molecule that encodes (a) a chimeric protein comprising a transcription factor fused to a domain comprising an inducible nuclear localization signal; and (b) an independently expressed selectable marker, wherein the isolated nucleic acid is DNA plasmid pBeaconRFP_GR, which comprises the nucleotide sequence of SEQ ID NO: 1.

In one embodiment, the present invention is directed to a host cell comprising an isolated nucleic acid molecule that encodes (a) a chimeric protein comprising a transcription factor fused to a domain comprising an inducible nuclear localization signal; and (b) an independently expressed selectable marker.

In one embodiment, the present invention is directed to a host cell comprising an isolated nucleic acid molecule that encodes (a) a chimeric protein comprising a transcription factor fused to a domain comprising an inducible nuclear localization signal; and (b) an independently expressed selectable marker, wherein the host cell is a plant protoplast.

In one embodiment, the present invention is directed to a host cell comprising an isolated nucleic acid molecule that encodes (a) a chimeric protein comprising a transcription factor fused to a domain comprising an inducible nuclear localization signal; and (b) an independently expressed selectable marker, wherein the host cell is a plant protoplast, and wherein the plant protoplast is derived from one of the following genuses: Acorus, Aegilops, Allium, Amborella, Antirrhinum, Apium, Arabidopsis, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

In one embodiment, the present invention is directed to a host cell comprising an isolated nucleic acid molecule that encodes (a) a chimeric protein comprising a transcription factor fused to a domain comprising an inducible nuclear localization signal; and (b) an independently expressed selectable marker, wherein the host cell is transfected with the nucleic acid molecule.

In one embodiment, the present invention is directed to a host cell comprising an isolated nucleic acid molecule that encodes (a) a chimeric protein comprising a transcription factor fused to a domain comprising an inducible nuclear localization signal; and (b) an independently expressed selectable marker, wherein the host cell is transiently transfected with the nucleic acid molecule.

In one embodiment, the present invention is directed to a host cell comprising an isolated nucleic acid molecule that encodes (a) a chimeric protein comprising a transcription factor fused to a domain comprising an inducible nuclear localization signal; and (b) an independently expressed selectable marker, wherein the host cell is derived from a genus that is different from the genus from which the transcription factor is derived from.

In one embodiment, the present invention is directed to a host cell comprising an isolated nucleic acid molecule that encodes (a) a chimeric protein comprising a transcription factor fused to a domain comprising an inducible nuclear localization signal; and (b) an independently expressed selectable marker, wherein the host cell is a plant protoplast derived from the genus Arabidopsis and the transcription factor is derived from the genus Zea.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor; and (v) identifying direct target genes of the transcription factor using a method comprising: (a) contacting the host cells with cyclohexamide; and (b) detecting the level of mRNA expressed in the host cells; wherein an alteration in the level of the mRNA expressed in the host cells treated with cyclohexamide compared to the level of the mRNA expressed in the host cells not treated with cyclohexamide indicates the identification of direct target genes of the transcription factor.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, wherein the host cell is a plant protoplast.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor; and (v) identifying direct target genes of the transcription factor using a method comprising: (a) contacting the host cells with cyclohexamide; and (b) detecting the level of mRNA expressed in the host cells; wherein an alteration in the level of the mRNA expressed in the host cells treated with cyclohexamide compared to the level of the mRNA expressed in the host cells not treated with cyclohexamdie indicates the identification of direct target genes of the transcription factor, wherein the host cell is a plant protoplast.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, wherein the host cell is a plant protoplast derived from one of the following genuses: Acorus, Aegilops, Allium, Amborella, Antirrhinum, Apium, Arabidopsis, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor; and (v) identifying direct target genes of the transcription factor using a method comprising: (a) contacting the host cells with cyclohexamide; and (b) detecting the level of mRNA expressed in the host cells; wherein an alteration in the level of the mRNA expressed in the host cells treated with cyclohexamide compared to the level of the mRNA expressed in the host cells not treated with cyclohexamide indicates the identification of direct target genes of the transcription factor, wherein the host cell is a plant protoplast derived from one of the following genuses: Acorus, Aegilops, Allium, Amborella, Antirrhinum, Apium, Arabidopsis, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, wherein the host cells are transiently transfected with the nucleic acid molecules.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, wherein the agent that induces nuclear localization of the chimeric protein is dexamethasone.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, wherein the step of detecting host cells that express the selectable marker is performed by Fluorescence Activated Cell Sorting (FACS).

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, wherein the step of detecting the level of mRNA expressed in the host cells is performed by quantitative PCR, high throughput sequencing, or gene microarrays.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, wherein the host cell is derived from a genus that is different from the genus from which the transcription factor is derived from.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, wherein the host cell is a plant protoplast derived from the genus Arabidopsis and the transcription factor is derived from the genus Zea.

In one embodiment, the present invention is directed to a method for identifying target genes of a transcription factor comprising: (i) transfecting plant protoplasts with a DNA plasmid that encodes (a) a chimeric protein comprising a transcription factor fused to a glucocorticoid receptor; and (b) an independently expressed red fluorescent protein; (ii) detecting the plant protoplasts that express the red fluorescent protein by performing Fluorescence Activated Cell Sorting.(FACS); (iii) contacting the plant protoplasts that express the red fluorescent protein with an dexamethasone; and (iv) detecting the level of mRNA expressed in the host cells, wherein an alteration in the level of the mRNA expressed in the plant protoplasts that have nuclear localization of the chimeric protein compared to the level of the mRNA expressed in the plant protoplasts that do not have nuclear localization of the chimeric protein indicates the identification of target genes of the transcription factor.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor; and (v) detecting transcription factor binding to genomic DNA in the host cells.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; and (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, and wherein the transcription factor is not ABI3.

In one embodiment, the present invention is directed to a method for identifying target genes of a transcription factor comprising: (i) transfecting host cells with a nucleic acid molecule described above; (ii) detecting host cells that express the selectable marker; (iii) contacting the host cells that express the selectable marker with an agent that induces nuclear localization of the chimeric protein; (iv) detecting the level of mRNA expressed in the host cells, wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor; and (v) detecting transcription factor binding to genomic DNA in the host cells, wherein the transcription factor is not ABI3.

### 3.1. TERMINOLOGY

Units, prefixes, and symbols may be denoted in their SI accepted form. Unless otherwise indicated, nucleic acids are written left to right in 5′ to 3′ orientation; amino acid sequences are written left to right in amino to carboxyl orientation, respectively. Numeric ranges recited within the specification are inclusive of the numbers defining the range and include each integer within the defined range. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

As used herein, the term “agronomic” includes, but is not limited to, changes in root size, vegetative yield, seed yield or overall plant growth. Other agronomic properties include factors desirable to agricultural production and business.

By “amplified” is meant the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., 1993, American Society for Microbiology, Washington, D.C. The product of amplification is termed an amplicon.

As used herein, “antisense orientation” includes reference to a duplex polynucleotide sequence that is operably linked to a promoter in an orientation where the antisense strand is transcribed. The antisense strand is sufficiently complementary to an endogenous transcription product such that translation of the endogenous transcription product is often inhibited.

In its broadest sense, a “delivery system,” as used herein, is any vehicle capable of facilitating delivery of a nucleic acid (or nucleic acid complex) to a cell and/or uptake of the nucleic acid by the cell.

The term “ectopic” is used herein to mean abnormal subcellular (e.g., switch between organellar and cytosolic localization), cell-type, tissue-type and/or developmental or temporal expression (e.g., light/dark) patterns for the particular gene or enzyme in question. Such ectopic expression does not necessarily exclude expression in tissues or developmental stages normal for said enzyme but rather entails expression in tissues or developmental stages not normal for the said enzyme.

By “endogenous nucleic acid sequence” and similar terms, it is intended that the sequences are natively present in the recipient plant genome and not substantially modified from its original form.

The term “exogenous nucleic acid sequence” as used herein refers to a nucleic acid foreign to the recipient plant host or, native to the host if the native nucleic acid is substantially modified from its original form. For example, the term includes a nucleic acid originating in the host species, where such sequence is operably linked to a promoter that differs from the natural or wild-type promoter.

By “encoding” or “encoded”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code. However, variants of the universal code, such as are present in some plant, animal, and fungal mitochondria, the bacterium Mycoplasma capricolum, or the ciliate Macronucleus, may be used when the nucleic acid is expressed therein.

When the nucleic acid is prepared or altered synthetically, advantage can be taken of known codon preferences of the intended host where the nucleic acid is to be expressed. For example, although nucleic acid sequences of the present invention may be expressed in both monocotyledonous and dicotyledonous plant species, sequences can be modified to account for the specific codon preferences and GC content preferences of monocotyledons or dicotyledons as these preferences have been shown to differ (Murray et al., 1989, Nucl. Acids Res. 17: 477-498). Thus, the maize preferred codon for a particular amino acid may be derived from known gene sequences from maize. Maize codon usage for 28 genes from maize plants is listed in Table 4 of Murray et al., supra.

By “fragment” is intended a portion of the nucleotide sequence. Fragments of the modulator sequence will generally retain the biological activity of the native suppressor protein. Alternatively, fragments of the targeting sequence may or may not retain biological activity. Such targeting sequences may be useful as hybridization probes, as antisense constructs, or as co-suppression sequences. Thus, fragments of a nucleotide sequence may range from at least about 20 nucleotides, about 50 nucleotides, about 100 nucleotides, and up to the full-length nucleotide sequence of the invention.

As used herein “full-length sequence” in reference to a specified polynucleotide or its encoded protein means having the entire amino acid sequence of, a native (non-synthetic), endogenous, biologically active form of the specified protein. Methods to determine whether a sequence is full-length are well known in the art including such exemplary techniques as northern or western blots, primer extension, 51 protection, and ribonuclease protection. See, e.g., Plant Molecular Biology: A Laboratory Manual, Clark, Ed., 1997, Springer-Verlag, Berlin. Comparison to known full-length homologous (orthologous and/or paralogous) sequences can also be used to identify full-length sequences of the present invention. Additionally, consensus sequences typically present at the 5′ and 3′ untranslated regions of mRNA aid in the identification of a polynucleotide as full-length. For example, the consensus sequence ANNNNAUGG, where the underlined codon represents the N-terminal methionine, aids in determining whether the polynucleotide has a complete 5′ end. Consensus sequences at the 3′ end, such as polyadenylation sequences, aid in determining whether the polynucleotide has a complete 3′ end.

The term “gene activity” refers to one or more steps involved in gene expression, including transcription, translation, and the functioning of the protein encoded by the gene.

The term “genetic modification” as used herein refers to the introduction of one or more exogenous nucleic acid sequences as well as regulatory sequences, into one or more plant cells, which in certain cases can generate whole, sexually competent, viable plants. The term “genetically modified” or “genetically engineered” as used herein refers to a plant which has been generated through the aforementioned process. Genetically modified plants of the invention are capable of self-pollinating or cross-pollinating with other plants of the same species so that the foreign gene, carried in the germ line, can be inserted into or bred into agriculturally useful plant varieties.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

By “host cell” is meant a cell that contains a vector and supports the replication and/or expression of the vector. Host cells may be prokaryotic cells such as E. coli, or eukaryotic cells such as yeast, plant, insect, amphibian, or mammalian cells. Preferably, host cells are monocotyledonous or dicotyledonous plant cells. A particularly preferred monocotyledonous host cell is a maize host cell.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “isolated” refers to material, such as a nucleic acid or a protein, which is: (1) substantially or essentially free from components which normally accompany or interact with it as found in its natural environment. The isolated material optionally comprises material not found with the material in its natural environment; or (2) if the material is in its natural environment, the material has been synthetically altered or synthetically produced by deliberate human intervention and/or placed at a different location within the cell. The synthetic alteration or creation of the material can be performed on the material within or apart from its natural state. For example, a naturally-occurring nucleic acid becomes an isolated nucleic acid if it is altered or produced by non-natural, synthetic methods, or if it is transcribed from DNA which has been altered or produced by non-natural, synthetic methods. See, e.g., Compounds and Methods for Site Directed Mutagenesis in Eukaryotic Cells, Kmiec, U.S. Pat. No. 5,565,350; In vivo Homologous Sequence Targeting in Eukaryotic Cells; Zarling et al., PCT/US93/03868. The isolated nucleic acid may also be produced by the synthetic re-arrangement (“shuffling”) of a part or parts of one or more allelic forms of the gene of interest. Likewise, a naturally-occurring nucleic acid (e.g., a promoter) becomes isolated if it is introduced to a different locus of the genome. Nucleic acids which are “isolated,” as defined herein, are also referred to as “heterologous” nucleic acids.

As used herein, the term “marker” refers to a gene encoding a trait or a phenotype which permits the selection of, or the screening for, a plant or plant cell containing the marker.

As used herein, “nucleic acid” includes reference to a deoxyribonucleotide or ribonucleotide polymer, or chimeras thereof, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

By “nucleic acid library” is meant a collection of isolated DNA or RNA molecules which comprise and substantially represent the entire transcribed fraction of a genome of a specified organism or of a tissue from that organism. Construction of exemplary nucleic acid libraries, such as genomic and cDNA libraries, is taught in standard molecular biology references such as Berger and Kimmel, Guide to Molecular Cloning Techniques, Methods in Enzymology, Vol. 152, Academic Press, Inc., San Diego, Calif. (Berger); Sambrook et al., 1989, Molecular Cloning—A Laboratory Manual, 2nd ed., Vol. 1-3; and Current Protocols in Molecular Biology, F. M. Ausubel et al., Eds., 1994, Current Protocols, a joint venture between Greene Publishing Associates, Inc. and John Wiley & Sons, Inc.

As used herein “operably linked” includes reference to a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame.

The term “orthologous” as used herein describes a relationship between two or more polynucleotides or proteins. Two polynucleotides or proteins are “orthologous” to one another if they are derived from a common ancestral gene and serve a similar function in different organisms. In general, orthologous polynucleotides or proteins will have similar catalytic functions (when they encode enzymes) or will serve similar structural functions (when they encode proteins or RNA that form part of the ultrastructure of a cell).

The term “overexpression” is used herein to mean above the normal expression level in the particular tissue, all and/or developmental or temporal stage for said enzyme/expressed protein product.

As used herein, the term “plant” is used in its broadest sense, including, but is not limited to, any species of woody, ornamental or decorative, crop or cereal, fruit or vegetable plant, and algae (e.g., Chlamydomonas reinhardtii). Non-limiting examples of plants include plants from the genus Arabidopsis or the genus Oryza. Other examples include plants from the genuses Acorus, Aegilops, Allium, Amborella, Antirrhinum, Apium, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.” Plants included in the invention are any plants amenable to transformation techniques, including gymnosperms and angiosperms, both monocotyledons and dicotyledons. Examples of monocotyledonous angiosperms include, but are not limited to, asparagus, field and sweet corn, barley, wheat, rice, sorghum, onion, pearl millet, rye and oats and other cereal grains. Examples of dicotyledonous angiosperms include, but are not limited to tomato, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers and various ornamentals. Examples of woody species include poplar, pine, sequoia, cedar, oak, etc. Still other examples of plants include, but are not limited to, wheat, cauliflower, tomato, tobacco, corn, petunia, trees, etc. As used herein, the term “cereal crop” is used in its broadest sense. The term includes, but is not limited to, any species of grass, or grain plant (e.g., barley, corn, oats, rice, wild rice, rye, wheat, millet, sorghum, triticale, etc.), non-grass plants (e.g., buckwheat flax, legumes or soybeans, etc.). As used herein, the term “crop” or “crop plant” is used in its broadest sense. The term includes, but is not limited to, any species of plant or algae edible by humans or used as a feed for animals or used, or consumed by humans, or any plant or algae used in industry or commerce. As used herein, the term “plant” also refers to either a whole plant, a plant part, or organs (e.g., leaves, stems, roots, etc.), a plant cell, or a group of plant cells, such as plant tissue, plant seeds and progeny of same. Plantlets are also included within the meaning of “plant.” The class of plants which can be used in the methods of the invention is generally as broad as the class of higher plants amenable to transformation techniques, including both monocotyledonous and dicotyledonous plants.

The term “plant cell” as used herein refers to protoplasts, gamete producing cells, and cells which regenerate into whole plants. Plant cell, as used herein, further includes, without limitation, cells obtained from or found in: seeds, suspension cultures, embryos, meristematic regions, callus tissue, leaves, roots, shoots, gametophytes, sporophytes, pollen, and microspores. Plant cells can also be understood to include modified cells, such as protoplasts, obtained from the aforementioned tissues.

As used herein, “polynucleotide” includes reference to a deoxyribopolynucleotide, ribopolynucleotide, or chimeras or analogs thereof that have the essential nature of a natural deoxy- or ribo-nucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically-, enzymatically- or metabolically-modified forms of polynucleotides, as well as the chemical forms of DNA and RNA characteristic of viruses and cells, including among other things, simple and complex cells.

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally-occurring amino acid, as well as to naturally-occurring amino acid polymers. The essential nature of such analogues of naturally-occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulfation, gamma-carboxylation of glutamic acid residues, hydroxylation and ADP-ribosylation. Further, this invention contemplates the use of both the methionine-containing and the methionine-less amino terminal variants of the protein of the invention.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such Agrobacterium or Rhizobium. Examples of promoters under developmental control include promoters that preferentially initiate transcription in certain tissues, such as leaves, roots, or seeds. Such promoters are referred to as “tissue preferred.” Promoters which initiate transcription only in certain tissue are referred to as “tissue specific.” A “cell type” specific promoter primarily drives expression in certain cell types in one or more organs, for example, vascular cells in roots or leaves. An “inducible” or “repressible” promoter is a promoter which is under environmental control. Examples of environmental conditions that may affect transcription by inducible promoters include anaerobic conditions or the presence of light. Tissue specific, tissue preferred, cell type specific, and inducible promoters represent the class of “non-constitutive” promoters. A “constitutive” promoter is a promoter which is active under most environmental conditions.

As used herein “recombinant” includes reference to a cell or vector that has been modified by the introduction of a heterologous nucleic acid, or to a cell derived from a cell so modified. Thus, for example, recombinant cells express genes that are not found in identical form within the native (non-recombinant) form of the cell, or exhibit altered expression of native genes, as a result of deliberate human intervention. The term “recombinant” as used herein does not encompass the alteration of the cell or vector by events (e.g., spontaneous mutation, natural transformation, transduction, or transposition) occurring without deliberate human intervention.

As used herein, a “recombinant expression cassette” is a nucleic acid construct, generated recombinantly or synthetically, with a series of specified nucleic acid elements which permit transcription of a particular nucleic acid in a host cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid to be transcribed, and a promoter.

The term “regulatory sequence” as used herein refers to a nucleic acid sequence capable of controlling the transcription of an operably associated gene. Therefore, placing a gene under the regulatory control of a promoter or a regulatory element means positioning the gene such that the expression of the gene is controlled by the regulatory sequence(s). Because a microRNA binds to its target, it is a post transcriptional mechanism for regulating levels of mRNA. Thus, an miRNA can also be considered a “regulatory sequence” herein. Not just transcription factors.

The term “residue” or “amino acid residue” or “amino acid” are used interchangeably herein to refer to an amino acid that is incorporated into a protein, polypeptide, or peptide (collectively “protein”). The amino acid may be a naturally occurring amino acid and, unless otherwise limited, may encompass non-natural analogs of natural amino acids that can function in a similar manner as naturally occurring amino acids.

The term “tissue-specific promotor” is a polynucleotide sequence that specifically binds to transcription factors expressed primarily or only in such specific tissue.

The term “selectively hybridizes” includes reference to hybridization, under stringent hybridization conditions, of a nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, preferably 90% sequence identity, and most preferably 100% sequence identity (i.e., complementary) with each other.

As used herein, a “stem-loop motif” or a “stem-loop structure,” sometimes also referred to as a “hairpin structure,” is given its ordinary meaning in the art, i.e., in reference to a single nucleic acid molecule having a secondary structure that includes a double-stranded region (a “stem” portion) composed of two regions of nucleotides (of the same molecule) forming either side of the double-stranded portion, and at least one “loop” region, comprising uncomplemented nucleotides (i.e., a single-stranded region).

The term “stringent conditions” or “stringent hybridization conditions” includes reference to conditions under which a probe will selectively hybridize to its target sequence, to a detectably greater degree than to other sequences (e.g., at least 2-fold over background). Stringent conditions are sequence-dependent and will be different in different circumstances. By controlling the stringency of the hybridization and/or washing conditions, target sequences can be identified which are 100% complementary to the probe (homologous probing). Alternatively, stringency conditions can be adjusted to allow some mismatching in sequences so that lower degrees of similarity are detected (heterologous probing). Generally, a probe is less than about 1000 nucleotides in length, optionally less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C.

Specificity is typically the function of post-hybridization washes, the critical factors being the ionic strength and temperature of the final wash solution. For DNA-DNA hybrids, the Tₘ can be approximated from the equation of Meinkoth and Wahl, 1984, Anal. Biochem., 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired identity. For example, if sequences with ≧90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); low stringency conditions can utilize a hybridization and/or wash at 11, 12, 13, 14, 15, or 20° C. lower than the thermal melting point (Tₘ). Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution) it is preferred to increase the SSC concentration so that a higher temperature can be used. An extensive guide to the hybridization of nucleic acids is found in Tijssen, 1993, Laboratory Techniques in Biochemistry and Molecular Biology—Hybridization with Nucleic Acid Probes, Part I, Chapter 2 “Overview of principles of hybridization and the strategy of nucleic acid probe assays”, Elsevier, New York; and Current Protocols in Molecular Biology, Chapter 2, Ausubel et al., Eds., 1995, Greene Publishing and Wiley-Interscience, New York. Hybridization and/or wash conditions can be applied for at least 10, 30, 60, 90, 120, or 240 minutes.

As used herein, “transcription factor” (“TF”) includes reference to a protein which interacts with a DNA regulatory element to affect expression of a structural gene or expression of a second regulatory gene. “Transcription factor” may also refer to the DNA encoding said transcription factor protein. The function of a transcription factor may include activation or repression of transcription initiation.

The term “transfection,” as used herein, refers to the introduction of a nucleic acid into a cell. The term “transient transfection,’ as used herein, refers to the introduction of a nucleic acid into a cell, wherein the nucleic acids introduced into the transfected cell are not permanently incorporated into the cellular genome.

As used herein, “transgenic plant” includes reference to a plant which comprises within its genome a heterologous polynucleotide or which lacks, by means of homologous recombination or other methods, a native polynucleotide. Generally, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to include any cell, cell line, callus, tissue, plant part or plant, the genotype of which has been altered by the presence of heterologous nucleic acid or lacks a native nucleic acid including those transgenics initially so altered as well as those created by sexual crosses or asexual propagation from the initial transgenic. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

The term “underexpression” is used herein to mean below the normal expression level in the particular tissue, all and/or developmental or temporal stage for said enzyme/expressed protein product.

As used herein, “vector” includes reference to a nucleic acid used in introduction of a polynucleotide of the present invention into a host cell. Vectors are often replicons. Expression vectors permit transcription of a nucleic acid inserted therein.

The following terms are used to describe the sequence relationships between a polynucleotide/polypeptide of the present invention with a reference polynucleotide/polypeptide: (a) “reference sequence”, (b) “comparison window”, (c) “sequence identity”, and (d) “percentage of sequence identity”.

(a) As used herein, “reference sequence” is a defined sequence used as a basis for sequence comparison with a polynucleotide/polypeptide of the present invention. A reference sequence may be a subset or the entirety of a specified sequence; for example, as a segment of a full-length cDNA or gene sequence, or the complete cDNA or gene sequence.

(b) As used herein, “comparison window” includes reference to a contiguous and specified segment of a polynucleotide/polypeptide sequence, wherein the polynucleotide/polypeptide sequence may be compared to a reference sequence and wherein the portion of the polynucleotide/polypeptide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides/amino acids residues in length, and optionally can be 30, 40, 50, 100, or longer. Those of skill in the art understand that to avoid a high similarity to a reference sequence due to inclusion of gaps in the polynucleotide/polypeptide sequence, a gap penalty is typically introduced and is subtracted from the number of matches.

Methods of alignment of sequences for comparison are well-known in the art. Optimal alignment of sequences for comparison may be conducted by the local homology algorithm of Smith and Waterman, 1981, Adv. Appl. Math. 2: 482; by the homology alignment algorithm of Needleman and Wunsch, 1970, J. Mol. Biol. 48: 443; by the search for similarity method of Pearson and Lipman, 1988, Proc. Natl. Acad. Sci. 85: 2444; by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, 1988, Gene 73: 237-244; Higgins and Sharp, 1989, CABIOS 5: 151-153; Corpet et al., 1988, Nucleic Acids Research 16: 10881-90; Huang et al., 1992, Computer Applications in the Biosciences 8: 155-65; and Pearson et al., 1994, Methods in Molecular Biology 24: 307-331.

The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel et al., Eds., 1995, Greene Publishing and Wiley-Interscience, New York.

Software for performing BLAST analyses is publicly available, e.g., through the National Center for Biotechnology Information (world-wide web at ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, 1989, Proc. Natl. Acad. Sci. USA 89:10915).

In addition to calculating percent sequence identity, the BLAST algorithm also performs a statistical analysis of the similarity between two sequences (see, e.g., Karlin & Altschul, 1993, Proc. Natl. Acad. Sci. USA 90:5873-5877). One measure of similarity provided by the BLAST algorithm is the smallest sum probability (P(N)), which provides an indication of the probability by which a match between two nucleotide or amino acid sequences would occur by chance.

BLAST searches assume that proteins can be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, 1993, Comput. Chem., 17:149-163) and XNU (Clayerie and States, 1993, Comput. Chem., 17:191-201) low-complexity filters can be employed alone or in combination.

Unless otherwise stated, nucleotide and protein identity/similarity values provided herein are calculated using GAP (GCG Version 10) under default values.

GAP (Global Alignment Program) can also be used to compare a polynucleotide or polypeptide of the present invention with a reference sequence. GAP uses the algorithm of Needleman and Wunsch (J. Mol. Biol. 48: 443-453, 1970) to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 100. Thus, for example, the gap creation and gap extension penalties can each independently be: 0, 1, 2, 3, 4, 5, 6, 7, 8, 9, 10, 15, 20, 30, 40, 50, 60 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff & Henikoff, 1989, Proc. Natl. Acad. Sci. USA 89:10915).

Multiple alignment of the sequences can be performed using the CLUSTAL method of alignment (Higgins and Sharp, 1989, CABIOS. 5:151-153) with the default parameters (GAP PENALTY=10, GAP LENGTH PENALTY=10). Default parameters for pairwise alignments using the CLUSTAL method are KTUPLE 1, GAP PENALTY=3, WINDOW=5 and DIAGONALS SAVED=5.

(c) As used herein, “sequence identity” or “identity” in the context of two nucleic acid or polypeptide sequences includes reference to the residues in the two sequences which are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, 1988, Computer Applic. Biol. Sci., 4:11-17, e.g., as implemented in the program PC/GENE (Intelligenetics, Mountain View, Calif., USA).

Polynucleotide sequences having “substantial identity” are those sequences having at least about 50%, 60% sequence identity, generally 70% sequence identity, preferably at least 80%, more preferably at least 90%, and most preferably at least 95%, compared to a reference sequence using one of the alignment programs described above. Preferably sequence identity is determined using the default parameters determined by the program. Substantial identity of amino acid sequences generally means sequence identity of at least 50%, more preferably at least 70%, 80%, 90%, and most preferably at least 95%. Nucleotide sequences are generally substantially identical if the two molecules hybridize to each other under stringent conditions.

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

As used herein, the term “transgenic,” when used in reference to a plant (i.e., a “transgenic plant”) refers to a plant that contains at least one heterologous gene in one or more of its cells, or that lacks at least one native gene, such as by means of homologous recombination, in one or more of its cells.

As used herein, “substantially complementary,” in reference to nucleic acids, refers to sequences of nucleotides (which may be on the same nucleic acid molecule or on different molecules) that are sufficiently complementary to be able to interact with each other in a predictable fashion, for example, producing a generally predictable secondary structure, such as a stem-loop motif. In some cases, two sequences of nucleotides that are substantially complementary may be at least about 75% complementary to each other, and in some cases, are at least about 80%, at least about 85%, at least about 90%, at least about 95%, at least about 96%, at least about 97%, at least about 98%, at least about 99%, at least about 99.5%, or 100% complementary to each other. In some cases, two molecules that are sufficiently complementary may have a maximum of 40 mismatches (e.g., where one base of the nucleic acid sequence does not have a complementary partner on the other nucleic acid sequence, for example, due to additions, deletions, substitutions, bulges, etc.), and in other cases, the two molecules may have a maximum of 30 mismatches, 20 mismatches, 10 mismatches, or 7 mismatches. In still other cases, the two sufficiently complementary nucleic acid sequences may have a maximum of 0, 1, 2, 3, 4, 5, or 6 mismatches.

By “variants” is intended substantially similar sequences. For “variant” nucleotide sequences, conservative variants include those sequences that, because of the degeneracy of the genetic code, encode the amino acid sequence of the modulator of the invention. Variant nucleotide sequences include synthetically derived sequences, such as those generated, for example, using site-directed mutagenesis. Generally, variants of a particular nucleotide sequence of the invention will have at least about 40%, 50%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters. By “variant” protein is intended a protein derived from the native protein by deletion or addition of one or more amino acids to the N-terminal and/or C-terminal end of the native protein; deletion or addition of one or more amino acids at one or more sites in the native protein; or substitution of one or more amino acids at one or more sites in the native protein. Such variants may result from, for example, genetic polymorphism or human manipulation. Conservative amino acid substitutions will generally result in variants that retain biological function

As used herein, the term “yield” or “plant yield” refers to increased plant growth, and/or increased biomass. In one embodiment, increased yield results from increased growth rate and increased root size. In another embodiment, increased yield is derived from shoot growth. In still another embodiment, increased yield is derived from fruit growth.

### 4. DESCRIPTION OF THE FIGURES

FIG. 1. Experimental scheme for TF and signal perturbation (A) and parallel RNA-Seq and ChIP-Seq analysis (B) of bZIP1 primary targets. (A) A GR::TF fusion protein is overexpressed in a protoplast and its location is restricted to the cytoplasm by Hsp90. DEX-treatment, releases the GR::TF from Hsp90 allowing TF entry to nucleus, where the TF binds and regulates its target genes (Bargmann et al., 2013, Molecular Plant 6(3):978; Eklund et al., 2010, Plant Cell 22:349). In the presence of CHX, translation is blocked so that gene expression level changes are caused solely by the TF association with primary targets, and not downstream effectors. (B) Prior to the GR::TF nuclear import, a pre-treatment with a signal (e.g. N) could result in post-translational modifications of the TF and/or transcriptional/post-translational effects on its TF partners (TF2). (C) Experimental design for temporal induction of TF and/or signal followed by identification of primary bZIP1 targets by either Microarray or ChIP-Seq analysis in the TARGET cell-based system (Bargmann et al., 2013, Molecular Plant 6(3):978). CHX: cycloheximide; DEX: dexamethasone; N: nitrogen; GR: glucocorticoid receptor.

FIG. 2. Diagram of the pBeaconRFP_GR vector. The pBeaconRFP_GR vector contains a red fluorescent protein (RFP) positive selection cassette and a Gateway recombination cassette that is in frame with the rat glucocorticoid receptor (GR) fusion protein. The plasmid is used to transfect protoplast suspensions, followed by treatment with dexamethasone and/or cycloheximide and cell-sorting of successful transformants for transcriptomic analysis.

FIG. 3. Preliminary analysis and microarray validation. (A) Timecourse qPCR analysis of PER1 and CRU3 induction by DEX in the presence of CHX. (B) The induction of six genes found to be significantly induced by ABI3 activation in the microarray was verified by qPCR analysis of independent transformations. Averages+/−SEM are presented, ns-not significant, **p<0.01, ***p<0.001 t-test DEX-treatment n=3.

FIG. 4. Promoter analysis of genes directly up-regulated by ABI3. (A) Spatial representation of RY-repeat, ABRE, G-box and bZIP-core CREs in the promoters of the 186 direct ABI3 up-regulated genes. Genes were ordered by fold induction. (B) Relative binding-site density distribution for the CREs in A 1000 bp upstream of the transcription start site in the 186 direct up-regulated genes. (C) Statistical overrepresentation of CREs in direct up-regulated genes. A sliding window of 30 genes was applied to calculate significance according to a hypergeometric test. Black dotted line indicates log fold change of the 186 genes. (D) The ABRE, G-box and bZIP-core elements.

FIG. 5. qPCR quantification of CRU3 transcript levels in protoplasts transformed with pBeaconRFP_GR-ABI3 or an empty vector control and treated with DEX and/or CHX. Averages+/−SEM are presented, ns-not significant, *p<0.05, ***p<0.001 t-test DEX-treatment n=3.

FIG. 6. qPCR quantification of PER1 transcript levels in protoplasts transformed with pBeaconRFP_GR-ABI3 or an empty vector control and treated with DEX and/or CHX. Averages+/−SEM are presented, ns-not significant, *p<0.05, ***p<0.001 t-test DEX-treatment n=3. FIG. 6. Proposed model of the interaction between the Arabidopsis circadian clock and N-assimilatory pathway. Arrows indicate influences that affect the function of the two processes. Black arrow: Clock function would affect N-assimilation. This influence is at least partly due to the direct regulatory role of CCA1 on N-assimilation. Grey arrow: N-assimilation would influence clock function through downstream metabolites such as Glu, Gln and possibly other N-metabolites.

FIG. 7. The intersection of 186 genes identified by TARGET as directly up-regulated by ABI3 and genes identified by previous studies as direct up-regulated targets of ABI3 (98 genes;), up-regulated targets of VP1 (51 genes) and ABI5 (59 genes).

FIG. 8. Network model of putative ABI3 connections to its direct up-regulated target genes via the RY-repeat motif (CATGCA) and through interaction with ABRE binding factors (ABFs) and ABRE (ACGTGKC) or the more degenerate G-box (CACGTG) and bZIP core (ACGTG) elements. Target genes (circles) are sized according to their strength of induction.

FIG. 9. Weight matrix representation of the ABRE-like (CACGTGKC) motif retrieved by the MotifSampler and MEME algorithms from the 1 kb upstream of the transcription start sites of the top fifty direct up-regulated ABI3 targets, Ze=7.19 and Ze=7.11, respectively.

FIG. 10. Identification of primary targets of bZIP1 by either Microarray or ChIP-Seq and integration of results. (A) Bioinformatics pipeline used to analyze the transcriptome data for transcriptionally regulated genes and the ChIP-Seq data for bZIP1-bound genes. Data from both sources were then integrated to decipher the binding and regulation dynamics. (B) Identification of primary targets regulated by bZIP1 in the presence of cycloheximide (to block secondary targets) and (C) their associated cis-regulatory motifs. (D) Identification of bZIP1-bound genes by ChIP-Seq (E) and their associated cis-regulatory motifs.

FIG. 11. Three distinct classes of bZIP1 primary targets identified by integration of microarray and ChIP-SEQ data (A) TF primary targets identified by either bZIP1-induced regulation in the presence of CHX (microarray) or bZIP1 binding (ChIP-SEQ) led to the identification of three distinct classes of bZIP1 primary targets: (I) “Poised” TF-bound but not regulated, (II) “Active” TF-bound and regulated, and (III) “Transient” TF-regulated but no binding, which can further be divided into subclasses based on the direction of regulation. Note that 187 bZIP1-bound TF-targets are not on the ATH1 microarray. The over-represented GO terms (FDR<0.01) for each subclass are listed. The significance of overlap with the N-responsive genes, or genes regulated by N*bZIP1 interaction was calculated for each subclass by hypergeometric distribution. (B) Comparison of the subclasses with previous reported bZIP1 regulated genes in planta (Kang et al., 2010, Molecular Plant 3:361), steady-state N-regulated genes (Gutierrez et al., 2008, Proc. Natl. Acad. Sci. U.S.A. 105:4939), and early/transient N-regulated genes (Krouk et al., 2010, Genome Biology 11:R123). (C) Enrichment of mRNA of different half-lives (Chiba et al., 2013, Plant & cell physiology. 54:180) in Class II and Class III of bZIP1 primary targets (filtered to only contain genes that are regulated by DEX in the presence and absence of CHX). The number of genes overlapping in each comparison is listed and the significance of the overlap is noted. Any overlap significance <0.01 is highlighted.

FIG. 12. A model for three modes of temporal TF Action of bZIP1 on primary target genes: “poised”, “active” and “transient”. This model illustrates temporal modes of action of bZIP1 with the three different classes of primary gene targets—I “poised”, II “active”, and III “transient” (A) and significantly over-represented cis-element motifs in each class (B). The significance of the over-representation of known bZIP binding motifs (hybrid ACGT box [ACG]ACGT[GC] (Kang et al., 2010, Molecular Plant 3:361) and GCN4 binding motif (Onodera et al., 2001, Journal of Biological Chemistry 276:14139)) are listed. The significance of specific cis-motifs enriched in each subclass, compared to other classes, is shown as a heat-map.

FIG. 13. Heatmap showing the expression profiles of nitrogen (N)-responsive genes in the TARGET cell-based system (Bargmann et al., 2013, Molecular Plant 6(3):978) identified by microarray. The GO terms over-represented (FDR adjusted pval<0.05) were identified for the N up-regulated and N down-regulated genes.

FIG. 14. Genes regulated in response to DEX treatment (i.e. DEX-induced TF nuclear import) (FDR<0.05) and with a significant N*DEX interaction (pval<0.01) from ANOVA analysis. (A) Heatmap showing four distinct clusters were observed and their significantly enriched GO terms are listed. (B) Gene regulatory network constructed from the genes in (A) and bZIP1 using Multinetwork feature in VirtualPlant (Katari et al., 2010, Plant Physiology 152:500).

FIG. 15. bZIP1 targets identified in this study validate the predicted bZIP1 targets based on network analysis of in planta N-treatment transcriptome data (Gutierrez et al., 2008, Proc. Natl. Acad. Sci. U.S.A. 105:4939). 27 genes were predicted to be the targets of bZIP1 of which 14 were confirmed by this study.

The comparison of the genes of the 5 subclasses with (A) DEX regulated genes in the absence of CHX and (B) previously reported Carbon (C)- and Light (L)-regulated gene lists identified from roots and shoots (Krouk et al., 2009, PLoS Computational Biology 5:e1000326). The number of genes overlapping in each comparison is listed and the significance of the overlap noted. A significance of overlap <0.01 is highlighted.

FIG. 17. Cis-regulatory motif analysis of the subclasses of bZIP1 target genes. The significance of over-representation of known cis-regulatory motifs were calculated for each subclass, and if the significance in at least one subclass is smaller than 0.01, the motif is listed and significance shown as a heatmap (A). From this collection of significant motifs, relatively enriched motifs in each subclass were selected by the pattern match algorithm PTM in Mev (B). The motifs enriched in the subgroups were also identified by PTM for the following subgroups: activated subgroup, repressed subgroup, bound and regulated subgroup, and no binding but regulated subgroup.

FIG. 18. Enrichment of mRNA of different half-lives (34) in Class II and Class III of bZIP1 primary target genes. The Class II and Class III genes here are filtered to only contain genes that are also regulated by DEX in the absence of CHX. Number of genes overlapping in each comparison is listed and the significance of the overlap noted. A significance of overlap <0.01 is highlighted.

FIG. 19. Schematic diagram of the data mining approach used in this study. Briefly, O. sativa (rice) and A. thaliana plants were grown for 12 days before treatment with nitrogen. Genome-wide analysis using Affymetrix chips has been used in order to quantify mRNA levels. Modeling of microarray data, using ANOVA and ortholog and network analysis (detailed in Methods), were used to identify a core translational network.

FIG. 20. Number of N-responsive genes in O. sativa and A. thaliana with ortholog information in the other species (*E-value cutoff 1e⁻²⁰).

FIG. 21. Flowchart of N-regulated rice core correlated network analysis process.

FIG. 22. NutriNet Modules: Constructing maize N-regulatory networks exploiting Arabidopsis Network Knowledge.

FIG. 23. A NutriNet Module: Core N-regulatory module conserved between maize and Arabidopsis includes previously validated transcription factor hubs (CCA1, GLK1, and bZIP) (Gutierrez et al., 2008, Proc Natl Acad Sci USA 105(12):4939; Baulcombe, 2010, Science 327(5967):761).

FIGS. 24 A-D. Experimental scheme for TF (A) and N-signal perturbation (B), and parallel RNA-Seq and ChIP-Seq analysis (C & D) of bZIP1 primary targets. (A) A GR::TF fusion protein is overexpressed in protoplasts and its location is restricted to the cytoplasm by Hsp90. DEX-treatment releases the GR::TF from Hsp90 allowing TF entry to the nucleus, where the TF binds to and regulates its target genes. CHX blocks translation. Thus, when DEX-induced TF import is performed in the presence of CHX, changes in transcript levels are attributed to the direct interaction of the target with the TF of interest. (B) Prior to DEX-induction of GR::TF nuclear import, pre-treatment with a signal (e.g. N-nutrient signal) could result in posttranslational modifications of the TF and/or transcriptional/post-translational effects on its TF partners (e.g. TF2). Genes whose response to TF-induced regulation (by DEX) is altered by CHX treatment were removed from the study to eliminate potential side effects of CHX. (C) Experimental design for identification of primary bZIP1 targets by either Microarray or ChIP-Seq analysis in the cell-based TARGET system (11, 26). CHX: cycloheximide; DEX: dexamethasone; N: nitrogen; GR: glucocorticoid receptor. (D) Bioinformatics pipeline to identify bZIP1 primary targets based on transcriptional response or TF binding. bZIP1-regulated genes were identified by ATH1 arrays. bZIP1-bound genes were identified by ChIP-Seq analysis. The integrated datasets were analyzed for the functional significance of classes of genes grouped based on TF-binding and/or TF-regulation.

FIG. 25. Nitrogen-responsive genes in the cell-based TARGET system. A heat map showing the expression profiles of 328 nitrogen (N)-responsive genes in the TARGET cell-based system as identified by microarray in this study. The GO terms over-represented (FDR adjusted p-val<0.05) were identified for the genes up-regulated or down-regulated in response to the N-signal perturbation.

FIG. 26. Validation of N-response in TARGET system. The 328 N-responsive genes in the cell-based TARGET system show significant overlaps with previously reported N-response gene in roots of whole plants and in seedlings. The significance of overlap between any two of these N-responsive sets is determined by the Genesect tool in VirtualPlant Platform (www.virtualplant.org).

FIGS. 27 A-D. Primary targets of bZIP1 are identified by either TF-activation or TF-binding. (A) Cluster analysis of bZIP1 primary target genes identified by their upregulation or down-regulation by DEX-induced bZIP1 nuclear import in Arabidopsis root protoplasts sequentially treated with inorganic N, CHX and DEX. bZIP motifs and other cismotifs are significantly over-represented in the promoters of bZIP1 primary target genes identified by transcriptional response (B), or by bZIP1 binding (D). (C) Examples of primary targets bound transiently by bZIP1 based on time-course ChIP-Seq.

FIG. 28. Genes influenced by a significant N-signal×bZIP1 interaction in the cell-based TARGET system. Genes regulated in response to DEX-induced bZIP1 nuclear import (FDR<0.05) and with a significant N-signal*bZIP1 interaction (p-val<0.01) from ANOVA analysis. Heat map showing four distinct clusters of genes regulated by a N-signal×bZIP1 interaction. Note that two of the “early response” genes shown to bind transiently to bZIP1 (NLP3 and LBD39, see FIG. 29C), are in cluster 1 of the genes regulated by a N-signal×bZIP1 interaction.

FIGS. 29 A-D. Class III transient targets of bZIP1 are uniquely associated with rapid N signaling. (A) Primary bZIP1 targets identified by either bZIP1-induced regulation or bZIP1-binding assayed in the same root protoplasts samples. Intersection of these datasets revealed three distinct classes of primary targets: (Class I) “Poised”, TF-bound but not regulated, (Class II) “Stable”, TF-bound and regulated, and (Class III) “Transient”, TF-regulated but no detectable binding. Classes II and III are subdivided into activated or repressed, with their associated over-represented GO terms (FDR<0.01) listed. (B) bZIP1 primary targets detected in protoplasts were compared with bZIP1 regulated genes in planta. The size of overlap is listed and significance is indicated by asterisks (highlight: p-val<0.001)). (C) bZIP1 primary targets detected in protoplasts were compared with and N-regulated genes in plants. The size of overlap is listed and significance is indicated by asterisks (highlight: p-val<0.001)). Class III “transient” targets are uniquely enriched in genes related to rapid N-signaling. (D) Class IIIA target genes (NLP3 and NRT2.1) show transient bZIP1 binding at 1 and 5 minutes after nuclear import of bZIP1, but not at later time-points (30 and 60 min).

FIG. 30. Class III bZIP1 transient targets are specifically enriched in co-inherited cis-motif elements. The significance of the over-representation of the known bZIP binding motifs hybrid ACGT box, and GCN4 binding motif, are listed for each class of bZIP1 primary targets. In addition to these bZIP binding sites, the significance of enrichment of co-inherited cis-regulatory motifs is shown as a heat-map specific to each subclass.

FIG. 31. Over-represented GO terms in each of the bZIP1 target classes. The set of genes from each class of bZIP1 targets were analyzed for over-representation of GO terms using the BioMaps feature of VirtualPlant (www.virtualplant.org). All classes of bZIP1 targets have an over-representation of GO terms related to “Stress” and “Stimulus”. When sub-divided by direction of regulation, Class IIA loses all significant GO terms. In addition to the stress terms, Class I is over-represented for genes responding to “biotic stress” and “divalent ion transport”. Class IIIA shows specific enrichment of GO terms for “Amino acid metabolism,” hence showing an enrichment of genes related to the N-signal. Class IIIB has specific enrichment of genes related to cell death and phosphorus metabolism.

FIG. 32. A network of biological processes represented by Class III transient bZIP1 targets. The set of genes from Class III “transient” bZIP1 targets were analyzed for over-representation of GO terms using the Bingo plugin in Cytoscape (Smoot et al., 2011, Bioinformatics 27(3):431-432). In addition to terms related to “Stress” and “Stimulus” which are found in all 3 classes of bZIP1 targets, the Class III transient targets also shows class-specific enrichment of GO terms both for “nitrogen metabolism” and the “regulation of nitrogen compound metabolism”, hence showing an enrichment of genes related to the N-signal. Class III transient targets also show overrepresentation of genes involved in “defense response”, “phosphorylation” and “regulation of metabolism.”

FIG. 33. bZIP1 as a pioneer TF for N-uptake/assimilation pathway genes. Global analysis of bZIP1 targets reveals that it regulates multiple genes encoding for the Nuptake/assimilation pathway. Multiple genes encoding nitrate transporters and isoenzymes in the N-assimilation pathway are represented by hexagonal nodes. The nodes targeted by bZIP1 are connected with red arrows. Thickness of the arrow is proportional to the number of genes in that node that are targeted by bZIP1. The IDs of the targeted genes are listed adjacent to the node. This pathway overview suggests that bZIP1 is a master regulator of the N-assimilation pathway. The pathway was constructed in Cytoscape (www.cytoscape.org) based on KEGG annotation (www.genome.jp/kegg/). Node abbreviations: NRT: Nitrate transporters; AMT: Ammonia transporters; GDH: Glutamate dehydrogenases; GOGAT: Glutamate synthases; GS: Glutamine synthetases; ASN: Asparagine synthetases.

FIG. 34. A “Hit-and-Run” transcription model enables bZIP1 to rapidly and catalytically activate genes in response to a N-signal. The transient mode-of-action for Class III bZIP1 targets follows a classic model for “hit-and-run” transcription. In this model, transient interactions of bZIP1 with Class III targets (the “hit”), lead to recruitment of the transcription machinery and possibly other TFs. Next, the transient nature of the bZIP1-target interaction (the “run”) enables bZIP1 to catalytically activate a large set of rapidly induced genes (e.g. target 2 . . . target n) biologically relevant to rapid transduction of the N-signal.

FIGS. 35 A-D. 4sU RNA tagging. (A) Dot blot showing that protoplasts are able to use 4sU for RNA synthesis in 20 min after the addition of 4sU. (B) Overlap of the actively transcribed genes regulated by bZIP1 (rows) with the three classes of bZIP1 targets (columns). The size of the overlap of two gene sets (labeled by the row and the column) was indicated by the numbers. The significance of overlap was indicated as: **: p<0.01; ***: p<0.001 (shade). (C). Time-series ChIP-seq showing the transient binding of bZIP1 to NLP3 at 1-5 min after nuclear import of bZIP1. (D) 4sU tagging showing that NLP3 is transcribed due to bZIP1 at both 20 min and 5 hr after nuclear import of bZIP1.

FIG. 36. Transient bZIP1 targets detected in TARGET cell-based system (inner circle) are predicted to regulate secondary targets of TF1 identified in planta (outer circle).

FIG. 37. The Network Walking Pipeline. Network inference links transient TF2 targets of TF1, detected only in the cell-based TARGET system, to secondary TF targets (gene Z) detected only by in planta TF1 perturbation.

### 5. DETAILED DESCRIPTION

The present invention involves plant genes that are regulated by transcription factors that control the gene network response to an environmental perturbation or signal (e.g., nitrogen, water, sunlight, oxygen, temperature). These genes respond rapidly to their environment, but surprisingly, there is no evidence of direct transcription factor interaction. More particularly, the large class of genes described herein (and exemplified in Tables 1, 2, 19, 20, and 23) respond to the perturbation of a regulatory transcription factor and the signal it transduces, but in fact are not stably bound to the transcription factor, and yet are most relevant to the signal induced in vivo—in other words, they represent members of the “dark matter” of metabolic regulatory circuits. In some embodiments, these “response genes” are transgenically manipulated so that their respective gene products are either overexpressed or underexpressed in a plant in order to confer a desired phenotype. In other embodiments, the genes encoding the transcription factors regulating these “response genes” are transgenically manipulated so that their respective gene products are either overexpressed or underexpressed in a plant in order to confer a desired phenotype. In a particular embodiment, the desired phenotype is increased nitrogen usage, which may be desired to enhance plant growth. In another embodiment, the desired phenotype is increased nitrogen storage, which may be desired to enhance the storage of nitrogen in seeds of seed crops. In yet other embodiments, the desired phenotype is

[symbol]

In certain embodiments, the transgenically manipulated response gene is one or more of the following (also listed in Tables 1 and 2): At3g28510, At1g73260, At1g22400, At1g80460, At1g05570, At5g22570, At5g65110, At1g24440, At5g04310, At3g16150, At4g13430, At1g08090, At5g57655, At1g62660, At3g14050, At5g18670, At1g15380, At5g56870, At2g43400, At3g28510, At1g73260, At1g22400, At1g80460, At1g05570, At5g22570, At5g65110, At1g24440, At5g04310, At3g16150, At4g13430, At1g08090, At5g57655, At1g62660, At3g14050, At5g18670, At1g15380, At5g56870, At2g43400, At3g28510, At1g73260, At1g22400, At1g80460, At1g05570, At5g22570, At5g65110, At1g24440, At5g04310, At3g16150, At4g13430, At1g08090, At5g57655, At1g62660, At3g14050, At5g18670, At1g15380, At5g56870, At2g43400, At3g28510, At1g73260, At1g22400, At1g80460, At1g05570, At5g22570, At5g65110, At1g24440, At5g04310, At3g16150, At4g13430, At1g08090, At5g57655, At1g62660, At3g14050, At5g18670, At1g15380, At5g56870, or At2g43400.

In certain embodiments, the transgenically manipulated TF is one or more of the following (also listed in Table 3): At1g01060, At1g01720, At1g13300, At1g15100, At1g22070, At1g25550, At1g25560, At1g29160, At1g43160, At1g51700, At1g51950, At1g53910, At1g66140, At1g68670, At1g68840, At1g74660, At1g74840, At1g75390, At1g77450, At1g80840, At2g04880, At2g20570, At2g22430, At2g22850, At2g24570, At2g25000, At2g28510, At2g28550, At2g30250, At2g33710, At2g38470, At2g46830, At3g01560, At3g04070, At3g06590, At3g20770, At3g25790, At3g46130, At3g47620, At3g51920, At3g54620, At3g60490, At3g61150, At3g61890, At3g62420, At4g17490, At4g17500, At4g24240, At4g27410, At4g31800, At4g34590, At4g36540, At4g37180, At4g37260, At4g37610, At4g37730, At5g05410, At5g06800, At5G10030, At5g13080, At5g14540, At5g24800, At5g39610, At5g44190, At5g47230, At5g48655, At5g49450, At5g49520, At5g56270, At5g60850, At5g63790, At5G65210, or At5g65640.

In certain embodiments, the transgenically manipulated plant is a species of woody, ornamental, decorative, crop, cereal, fruit, or vegetable. In other embodiments, the plant is a species of one of the following genuses: Acorus, Aegilops, Allium, Amborella, Antirrhimum, Apium, Arabidopsis, Arachis, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Peryea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

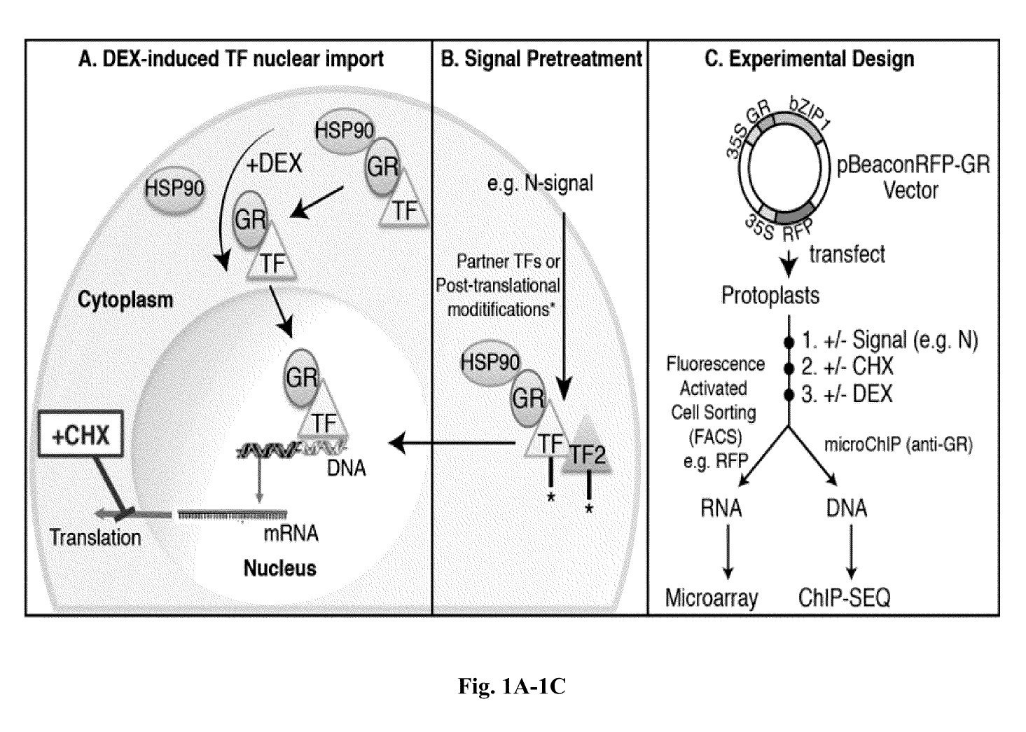

The invention is based, in part, on the development of a rapid technique named “TARGET” that uses transient expression of a glucocorticoid receptor (GR)-tagged TF in protoplasts to study the genome-wide effects of TF activation. In some embodiments, the TARGET system can retrieve information on direct target genes in less than two weeks time. Multiple experimental designs exist for use of the TARGET system, as shown in FIG. 1. In some embodiments, the present invention is directed to a method for identifying target genes of a transcription factor comprising: (i) transfecting host cells with an isolated nucleic acid molecule that encodes (a) a chimeric protein comprising a transcription factor fused to a domain comprising an inducible cellular localization signal; and (b) an independently expressed selectable marker; (ii) detecting host cells that express the selectable marker; (iii) contacting the host cells that express the selectable marker with an agent that induces localization (e.g. counters sequestration in the cytoplasm and/or targets to the nucleus, mitochondria, or chloroplasts) of the chimeric protein; and (iv) detecting the level of mRNA expressed in the host cells; wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor.

In certain embodiments, the method of the present invention further comprises identifying direct target genes of the transcription factor comprising: (v) contacting the host cells with cyclohexamide; and (vi) detecting the level of mRNA expressed in the host cells; wherein an alteration in the level of the mRNA expressed in the host cells treated with cyclohexamide compared to the level of the mRNA expressed in the host cells not treated with cyclohexamide indicates the identification of direct target genes of the transcription factor.

In some embodiments, the nucleic acid molecule utilized in the methods of the invention is a DNA plasmid. In some embodiments, the domain comprising an inducible cellular localization signal encoded by the nucleic acid molecule used in the method of the invention is glucocorticoid receptor and the agent that allows for nuclear localization of the chimeric protein is dexamethasone. Dexamethasone prevents sequestration of the GR-TF fusion in the cytoplasm, allowing for localization to the nucleus. In some embodiments, the cellular localization signal encoded by the nucleic acid molecule allows for localization to the chloroplast or mitochondria upon treatment with the inducing agent.

In one embodiment, a) an isolated nucleic acid encoding a GR-TF fusion construct and an independently expressed selectable marker (e.g. a fluorescent protein such as RFP) is transiently transfected into plant protoplasts; b) treatment of the protoplasts with dexamethasone releases the GR-TF fusion from sequestration in the cytoplasm, allowing the TF to reach target genes; c) protoplasts that have been transiently transfected are identified by means of the detectable signal gene (e.g. by fluorescence activated cell sorting (FACS) to determine the presence of a fluorescent protein such as RFP); d) mRNA transcripts are measured from the transiently transfected protoplasts through use of a microarray analysis.

In some embodiments, the protoplasts are optionally exposed to an environmental signal, such as nitrogen, before treatment with dexamethasone, allowing for the measurement of transcription factor activity in response to the signal. In some embodiments, protoplasts may optionally be treated with cyclohexamide prior to or concurrently with dexamethasone treatment, which blocks translation, allowing for the distinction of primary target genes, which are still expressed in the presence of cyclohexamide, from secondary target genes, which are not expressed in the presence of cyclohexamide. In some embodiments, TF binding to response genes in transiently transfected protoplasts may optionally be analyzed using ChIP-Seq. In some embodiments, ChIP-Seq or microarray analysis is performed at differing time points after an environmental signal in order to determine temporal changes in TF binding or gene expression.

In certain embodiments, gene networks are identified that are regulated by TFs which demonstrate only transient association with a target gene. The identified TFs that regulate a target gene but are only transiently associated with that target gene can be referred to as “touch and go” or “hit and run” TFs. Touch and go (hit and run) TFs are implicated when (i) one or more particular gene transcript levels are perturbed when the TF-fusion construct is transiently expressed and released from sequestration in the cytoplasm, and (ii) stable binding to the gene or genes is not detected by ChIP SEQ analysis. In some embodiments, these touch and go (hit and run) TFs regulate genes that control responsiveness to an environmental signal, perturbation, or cue. The identified genes targeted by these transiently-associating TFs in response to an environmental signal, perturbation, or cue can be referred to as “response genes.” “Response genes” are implicated when, in the presence of an environmental signal, perturbation, or cue, “touch and go” (hit and run) TFs perturb the levels of one or more particular gene transcript yet do not stably bind the gene as measured by ChIP-Seq analysis. The identification of a particular response gene or set of genes may vary with time after the protoplast is exposed to the environmental signal, perturbation, or cue.

The present invention uses nucleic acid molecules, compositions and methods for determining the target genes of transcription factors and the structure of gene regulatory networks (GRN) by transiently expressing transcription factors of interest in host cells, such as protoplasts. The protoplasts can be isolated and utilized from virtually any plant genus and species in the methods of the invention so that target genes and gene regulatory networks in poorly characterized plant genus and species can be studied. The methods of the invention allow for cross-species studies in order to analyze evolutionary conserved networks using genes from a poorly characterized plant genus or species in a better characterized model genus, such as Arabidopsis, which has a fully sequenced genome and has microarray chip data available. By providing the ability to do reciprocal cross species genetic network comparisons, the TARGET technique allows for the determination of what is evolutionary conserved and therefore likely the most important elements of transcription factor networks.

In some embodiments, the selectable marker encoded by the nucleic acid molecule used in the method of the invention is a fluorescent selection marker. A fluorescent selection marker that can be used in the method of the invention includes, but is not limited to, green fluorescent protein, yellow fluorescent protein, red fluorescent protein, cyan fluorescent protein, or blue fluorescent protein. In a specific embodiment, the fluorescent selection marker used in the method of the invention is red fluorescent protein. In certain embodiments, the step of detecting host cells that express the selectable marker is performed by Fluorescence Activated Cell Sorting (“FACS”).

In a specific embodiment, the nucleic acid molecule utilized in the methods of the invention is DNA plasmid pBeaconRFP_GR, which comprises the nucleotide sequence of SEQ ID NO: 1.

In certain embodiments, the host cell utilized in the methods of the present invention are transiently transfected with the nucleic acid molecules of the invention. In some embodiments, the host cell utilized in the methods of the present invention is a plant protoplast. In particular embodiments, the plant protoplast is derived from one of the following genuses: Acorus, Aegilops, Allium, Amborella, Antirrhinum, Apium, Arabidopsis, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia. In some embodiments, the host cell is derived from a genus that is different from the genus from which the transcription factor is derived from. For example, the host cell is a plant protoplast derived from the genus Arabidopsis and the transcription factor is derived from the genus Zea.

5.1. Response Genes and Transcription Factors

The tables below list transcription factors and response genes for which expression may be modified in transgenic plants to produce desired phenotypes. In Section 5.2, methods for the production of transgenic plants with modified expression of one or more of these genes are enumerated.

Table 1 shows 20 genes that are (1) ClassIIIA, i.e. no TF binding but TF-activated and (2) transiently upregulated by N. These genes are examples of “response” genes. Table 2 shows 14 genes that are (1) ClassIIIA, i.e. no binding but activated and (2) early (9-20 min) upregulated by N. These are also “response” genes. Table 3 lists “touch and go” (“hit and run”) transcription factors that may be utilized with the TARGET system to discover more response genes, which may be modified in transgenic plants to create a desired phenotype. Likewise, the transcription factor genes listed in Table 3 may themselves be modified in transgenic plants to create a desired phenotype.

TABLE 1

| PUB LOCUS | ANNOTATION |
|---|---|
| At3g28510 | P-loop containing nucleoside triphosphate hydrolases |
| | superfamily protein |
| At1g73260 | ATKTI1, KTI1, kunitz trypsin inhibitor 1 |
| At1g22400 | ATUGT85A1, UGT85A1, UDP-Glycosyltransferase |
| | superfamily protein |
| At1g80460 | GLI1, NHO1, Actin-like ATPase superfamily protein |
| At1g05570 | ATGSL06, ATGSL6, CALS1, GSL06, GSL6, callose |
| | synthase 1 |
| At5g22570 | ATWRKY38, WRKY38, WRKY DNA-binding protein |
| | 38 |
| At5g65110 | ACX2, ATACX2, acyl-CoA oxidase 2 |
| At1g24440 | RING/U-box superfamily protein |
| At5g04310 | Pectin lyase-like superfamily protein |
| At3g16150 | N-terminal nucleophile aminohydrolases (Ntn |
| | hydrolases superfamily protein) |
| At4g13430 | ATLEUC1, IIL1, isopropyl malate isomerase large |
| | subunit 1 |
| At1g08090 | ACH1, ATNRT2.1, ATNRT2:1, LIN1, NRT2, NRT2.1, |
| | NRT2:1, NRT2; 1AT, nitrate transporter 2:1 |
| At5g57655 | xylose isomerase family protein |
| At1g62660 | Glycosyl hydrolases family 32 protein |
| At3g14050 | AT-RSH2, ATRSH2, RSH2, RELA/SPOT homolog 2 |
| At5g18670 | BAM9, BMY3, beta-amylase 3 |
| At1g15380 | Lactoylglutathione lyase/glyoxalase I family protein |
| At5g56870 | BGAL4, beta-galactosidase 4 |
| At2g43400 | ETFQO, electron-transfer flavoprotein:ubiquinone |
| | oxidoreductase |

TABLE 2

| PUB LOCUS | ANNOTATION |
|---|---|
| At1g62660: | Glycosyl hydrolases family 32 protein |
| At3g49940: | LBD38, LOB domain-containing protein 38 |
| At5g10210: | CONTAINS InterPro DOMAIN/s: C2 calcium-dependent |
| | membrane targeting (InterPro:IPR000008); BEST |
| | Arabidopsis thaliana protein match is: unknown |
| | protein (TAIR:AT5G65030.1); Has 1807 Blast hits to |
| | 1807 proteins in 277 species: Archae - 0; Bacteria - |
| | 0; Metazoa - 736; Fungi - 347; Plants - 385; Viruses - |
| | 0; Other Eukaryotes - 339 (source: NCBI BLink). |
| At1g07150: | MAPKKK13, mitogen-activated protein kinase kinase |
| | kinase 13 |
| At3g20320: | TGD2, trigalactosyldiacylglycerol2 |
| At2g43400: | ETFQO, electron-transfer flavoprotein:ubiquinone |
| | oxidoreductase |
| At1g22400: | ATUGT85A1, UGT85A1, UDP-Glycosyltransferase |
| | superfamily protein |
| At1g05570: | ATGSL06, ATGSL6, CALS1, GSL06, GSL6, callose |
| | synthase 1 |
| At4g38490: | unknown protein; Has 30201 Blast hits to 17322 proteins |
| | in 780 species: Archae - 12; Bacteria - 1396; Metazoa - |
| | 17338; Fungi - 3422; Plants - 5037; Viruses - 0; Other |
| | Eukaryotes - 2996 (source: NCBI BLink). |
| At4g37540: | LBD39, LOB domain-containing protein 39 |
| At5g65110: | ACX2, ATACX2, acyl-CoA oxidase 2 |
| At5g04310: | Pectin lyase-like superfamily protein |
| At4g39780: | Integrase-type DNA-binding superfamily protein |
| At5g51550: | EXL3, EXORDIUM like 3 |

TABLE 3

| PUB LOCUS | Name/Symbol | Annotation |
|---|---|---|
| At1g01060 | myb-related transcription factor | LHY encodes a myb-related putative transcription |
| | (LHY) | factor involved in circadian rhythm along with another |
| | | myb transcription factor CCA1 |
| At1g01720 | putative transcriptional activator with | Belongs to a large family of putative transcriptional |
| | NAC domain (ANAC002) | activators with NAC domain. Transcript level |
| | | increases in response to wounding and abscisic acid. |
| | | ATAF1 attentuates ABA signaling and sythesis. |
| | | Mutants are hyposensitive to ABA |
| At1g13300 | HRS1 | Overexpression confers hypersensitivity to low |
| | | phosphate-elicited inhibition of primary root growth |
| At1g15100 | Ring-H2 finger A2A (RHA2A) | Encodes a putative RING-H2 finger protein RHA2a. |
| At1g22070 | bZIP1 family transcription factor | Encodes a transcription factor. Like Other TGA1a- |
| | (TGA3) | related factors, TGA3 has a highly conserved bZIP |
| | | region and exhibits similar DNA-binding properties. |
| At1g25550 | HHO3 | myb-like transcription factor family protein |
| At1g25560 | putative AP2-domain containing | Encodes a member of the RAV transcription factor |
| | transcription factor (TEM1) | family that contains AP2 and B3 binding domains. |
| | | Involved in the regulation of flowering under long |
| | | days. Loss of function results in early flowering. |
| | | Overexpression causes late flowering and repression |
| | | of expression of FT. TBDvel transcriptional regulator |
| | | involved in ethylene signaling. Promoter bound by |
| | | EIN3. EDF1 in turn, binds to promoter elements in |
| | | ethylene responsive genes. |
| At1g29160 | Dof-type zinc finger domain- | |
| | containing protein | |
| At1g43160 | AP2 domain-containing protein | encodes a member of the ERF (ethylene response |
| | RAP2.6 (RAP2.6) | factor) subfamily B-4 of ERF/AP2 transcription factor |
| | | family (RAP2.6). the protein contains one AP2 |
| | | domain |
| At1g51700 | Dof-type zinc finger domain- | Encodes dof zinc finger protein (adof1). |
| | containing protein (ADOF1) | |
| At1g51950 | IAA18, indole-3-acetic acid inducible | Auxin responsive |
| | 18 | |
| At1g53910 | AP2 domain-containing protein | Encodes a member of the ERF (ethylene response |
| | RAP2.12 (RAP2.12) | factor) subfamily B-2 of ERF/AP2 transcription factor |
| | | family (RAP2.12). the protein contains one AP2 |
| | | domain. There are 5 members in this subfamily |
| | | including RAP2.2 AND RAP2.12. Involved in oxygen |
| | | sensing. |
| At1g66140 | zinc finger protein 4 transcription | |
| | factor (ZFP4) | |
| At1g68670 | HHO2 | myb-like transcription factor family protein |
| At1g68840 | regulator of ATPase of the vacuolar | Rav2 is part of a complex that has been named |
| | membrane (RAV2) | ‘regulator of the (H+)-ATPase of the vacuolar and |
| | | endosomal membranes’ (RAVE) |
| At1g74660 | Mini zinc finger 1 transcription factor | |
| | (MIF1) | |
| At1g74840 | MYB | Homeodomain-like superfamily protein |
| At1g75390 | AtbZIP44, bZTP44, basic leucine- | |
| | zipper 44 | |
| At1g77450 | NAC45 | NAC domain containing protein 32 (NAC032); |
| | | FUNCTIONS IN: sequence-specific DNA binding |
| | | transcription factor activity; INVOLVED IN: |
| | | multicellular organismal development, regulation of |
| | | transcription |
| At1g80840 | WRKY40 | Pathogen-induced transcription factor. Binds W-box |
| | | sequences in vitro. Forms protein complexes with |
| | | itself and with WRKY40 and WRKY60. Coexpression |
| | | with WRKY18 or WRKY60 made plants more |
| | | susceptible to both P. syringae and B. cinerea. |
| At2g04880 | WRKY1 | Encodes WRKY1, a member of the WRKY |
| | | transcription factors in plants involved in disease |
| | | resistance, abiotic stress, senescence as well as in |
| | | some developmental processes. WRKY1 is involved |
| | | in the salicylic acid signaling pathway. The crystal |
| | | structure of the WRKY1 C-terminal domain revealed |
| | | a zinc-binding site and identified the DNA-binding |
| | | residues of WRKY1. |
| At2g20570 | golden2-like transcription factor | Encodes GLK1, Golden2-like 1, one of a pair of |
| | (GLK1) | partially redundant nuclear transcription factors that |
| | | regulate chloroplast development in a cell-autonomous |
| | | manner. GLK2, Golden2-like 2, is encoded by |
| | | At5g44190. GLK1 and GLK2 regulate the expression |
| | | of the photosynthetic apparatus. |
| At2g22430 | ATHB6 | Encodes a homeodomain leucine zipper class I (HD- |
| | | Zip I) protein that is a target of the protein |
| | | phosphatase ABI1 and regulates hormone responses in |
| | | Arabidopsis. |
| At2g22850 | AtbZIP6, bZIP6, basic leucine-zipper | |
| | 6 | |
| At2g24570 | WRKY17 | |
| At2g25000 | WRKY60 | Pathogen-induced transcription factor. Forms protein |
| | | complexes with itself and with WRKY40 |
| At2g28510 | Dof-type zinc finger domain | Dof-type zinc finger DNA-binding family protein |
| | containing protein | |
| At2g28550 | RAP2.7/TOE1 | related to AP2.7 (RAP2.7) |
| At2g30250 | WRKY25 | member of WRKY Transcription Factor; Group I. |
| | | Located in nucleus. Involved in response to various |
| | | abiotic stresses - especially salt stress |
| At2g33710 | AP2-33 | encodes a member of the ERF (ethylene response |
| | | factor) subfamily B-4 of ERF/AP2 transcription factor |
| | | family. The protein contains one AP2 domain |
| At2g38470 | WRKY33 | Member of the plant WRKY transcription factor |
| | | family. Regulates the antagonistic relationship |
| | | between defense pathways mediating responses to |
| | | P. syringae and necrotrophic fungal pathogens. Located |
| | | in nucleus. Involved in response to various abiotic |
| | | stresses - especially salt stress. |
| At2g46830 | myb-related transcription factor | Encodes a transcriptional represser that performs |
| | (CCA1) | overlapping functions with LHY in a regulatory |
| | | feedback loop that is closely associated with the |
| | | circadian oscillator of Arabidopsis. |
| At3g01560 | TTF1 | Ubiquitin-associated/translation elongation factor |
| | | EF1B, N-terminal |
| At3g04070 | NAC transcription factor family | NAC domain containing protein 47 (NAC047); |
| | (ANAC047) | FUNCTIONS IN: sequence-specific DNA binding |
| | | transcription factor activity; INVOLVED IN: |
| | | multicellular organismal development, regulation of |
| | | transcription |
| At3g06590 | Basic helix-loop-helix (bHLH) DNA | |
| | binding superfamily protein | |
| At3g20770 | EIN3 | Encodes EIN3 (ethylene-insensitive3), a nuclear |
| | | transcription factor that initiates downstream |
| | | transcriptional cascades for ethylene responses. |
| At3g25790 | HHO1 | myb-like transcription factor family protein |
| At3g46130 | ATMYB48, ATMYB48-1, | |
| | ATMYB48-2, ATMYB48-3, | |
| | MYB48, myb domain protein 48 | |
| At3g47620 | AtTCP14, TCP14, TEOSINTE | |
| | BRANCHED, cycloidea and PCF | |
| | (TCP) 14 | |
| At3g51920 | Calmodulin-like protein 9 (CAM9) | encodes a divergent member of calmodulin, which is |
| | | an EF-hand family of Ca2+-binding proteins. |
| At3g54620 | bZIP25 | |
| At3g60490 | Integrase-type DNA-binding | |
| | superfamily protein | |
| At3g61150 | HBZIP | Encodes a homeobox-leucine zipper family protein |
| | | belonging to the HD-ZIP IV family. |
| At3g61890 | ATHB12 | Encodes a homeodomain leucine zipper class I (HD- |
| | | Zip I) protein. Loss of function mutant has abnormally |
| | | shaped leaves and stems. |
| At3g62420 | bZIP53 | Encodes a group-S bZIP transcription factor. Forms |
| | | heterodimers with group-C bZIP transcription factors. |
| | | The heterodimers bind to the ACTCAT cis-element of |
| | | proline dehydrogenase gene. |
| At4g17490 | ethylene-responsive element binding | Encodes a member of the ERF (ethylene response |
| | factor 6 (ERF6) | factor) subfamily B-3 of ERF/AP2 transcription factor |
| | | family (ATERF-6). The protein contains one AP2 |
| | | domain. There are 18 members in this subfamily |
| | | including ATERF-1, ATERF-2, AND ATERF-5. It is |
| | | involved in the response to reactive oxygen species |
| | | and light stress. |
| At4g17500 | ethylene-responsive element-binding | Encodes a member of the ERF (ethylene response |
| | protein 1 (ERF1) | factor) subfamily B-3 of ERF/AP2 transcription factor |
| | | family (ATERF-1). The protein contains one AP2 |
| | | domain. |
| At4g24240 | WRKY7 | Encodes a Ca-dependent calmodulin binding protein. |
| | | Sequence similarity to the WRKY transcription factor |
| | | gene family. |
| At4g27410 | NAC transcription factor family | Encodes a NAC transcription factor induced in |
| | (RD26) | response to dessication. It is localized to the nucleus |
| | | and acts as a transcriptional activator in ABA- |
| | | mediated dehydration response. |
| At4g31800 | WRKY18 | Pathogen-induced transcription factor. Binds W-box |
| | | sequences in vitro. Forms protein complexes with |
| | | itself and with WRKY40 and WRKY60 |
| At4g34590 | ATB2, AtbZIP11, BZIP11, GBF6, G- | |
| | box binding factor 6 | |
| At4g36540 | BEE2, BR enhanced expression 2 | |
| At4g37180 | HHO5 | |
| At4g37260 | myb family transcription factor | Member of the R2R3 factor gene family. |
| | (MYB73) | |
| At4g37610 | BT5 | BTB and TAZ domain protein. Located in cytoplasm |
| | | and expressed in fruit, flower and leaves. |
| At4g37730 | AtbZIP7, bZIP7, basic leucine-zipper | |
| | 7 | |
| At5g05410 | DRE-binding protein 2A (DREB2A) | Encodes a transcription factor that specifically binds |
| | | to DRE/CRT cis elements (responsive to drought and |
| | | low-temperature stress). Belongs to the DREB |
| | | subfamily A-2 of ERF/AP2 transcription factor family |
| | | (DREB2A) |
| At5g06800 | myb-like HTH transcriptional | |
| | regulator family protein | |
| At5G10030 | TGA4 | |
| At5g13080 | ATWRKY75, WRKY75, WRKY | |
| | DNA-binding protein 75 | |
| At5g14540 | TTF2 | proline-rich family protein contains proline rich |
| | | extensin domains |
| At5g24800 | bZIP1 transcription factor family | Encodes bZIP protein BZO2H2. |
| | protein (bZIP9) | |
| At5g39610 | NAC6 | Encodes a NAC-domain transcription factor. |
| | | Positively regulates aging-induced cell death and |
| | | senescence in leaves. This gene is upregulated in |
| | | response to salt stress in wildtype as well as NTHK1 |
| | | transgenic lines although in the latter case the |
| | | induction was drastically reduced |
| At5g44190 | myb family transcription factor | Encodes GLK2, Golden2-like 2, one of a pair of |
| | (GLK2) | partially redundant nuclear transcription factors that |
| | | regulate chloroplast development in a cell-autonomous |
| | | manner. GLK1, Golden2-like 1, is encoded by |
| | | At2g20570. GLK1 and GLK2 regulate the expression |
| | | of the photosynthetic apparatus. |
| At5g47230 | AP2-6 | encodes a member of the ERF (ethylene response |
| | | factor) subfamily B-3 of ERF/AP2 transcription factor |
| | | family (ATERF-5). The protein contains one AP2 |
| | | domain |
| At5g48655 | C3HC4 RING | RING/U-box superfamily protein |
| At5g49450 | bZIP1 transcription factor family | Encodes a transcription activator is a positive |
| | protein (bZIP1) | regulator of plant tolerance to salt, osmotic and |
| | | drought stresses. |
| At5g49520 | ATWRKY48, WRKY48, WRKY | |
| | DNA-binding protein 48 | |
| At5g56270 | ATWRKY2, WRKY2, WRKY | |
| | DNA-binding protein 2 | |
| At5g60850 | Dof-type zinc finger domain | Encodes a zinc finger protein. |
| | containing protein (OBF4) | |
| At5g63790 | NAC transcription factor family | Encodes a member of the NAC family of transcription |
| | (ANAC102) | factors. ANAC102 appears to have a role in mediating |
| | | response to low oxygen stress (hypoxia) in |
| | | germinating seedlings. |
| At5G65210 | TGA1 | |
| At5g65640 | BHLH093 | beta HLH protein 93 (bHLH093) |

5.2. Transgenic Plants

5.2.1. Modulation of Gene Expression

The methods of the invention involve modulation of the expression of one, two, three or more target nucleotide sequences (i.e., target genes) in a host cell, such as a plant protoplast. That is, the expression of a target nucleotide sequence of interest may be increased or decreased.

The target nucleotide sequences may be endogenous or exogenous in origin. By “modulate expression of a target gene” is intended that the expression of the target gene is increased or decreased relative to the expression level in a host cell that has not been altered by the methods described herein.

By “increased or over expression” is intended that expression of the target nucleotide sequence is increased over expression observed in conventional transgenic lines for heterologous genes and over endogenous levels of expression for homologous genes. Heterologous or exogenous genes comprise genes that do not occur in the host cell of interest in its native state. Homologous or endogenous genes are those that are natively present in the plant genome. Generally, expression of the target sequence is substantially increased. That is expression is increased at least about 25%-50%, preferably about 50%-100%, more preferably about 100%, 200% and greater.

By “decreased expression” or “underexpression” it is intended that expression of the target nucleotide sequence is decreased below expression observed in conventional transgenic lines for heterologous genes and below endogenous levels of expression for homologous genes. Generally, expression of the target nucleotide sequence of interest is substantially decreased. That is expression is decreased at least about 25%-50%, preferably about 50%-100%, more preferably about 100%, 200% and greater.

Expression levels may be assessed by determining the level of a gene product by any method known in the art including, but not limited to determining the levels of the RNA and protein encoded by a particular target gene. For genes that encode proteins, expression levels may determined, for example, by quantifying the amount of the protein present in plant cells, or in a plant or any portion thereof. Alternatively, it desired target gene encodes a protein that has a known measurable activity, then activity levels may be measured to assess expression levels.

5.2.2. Transfection

Any method or delivery system may be used for the delivery and/or transfection of the nucleic acid vectors encoding any of the genes of interest of the present invention in the host cell, e.g., plant protoplast. The vectors may be delivered to the host cell either alone, or in combination with other agents. Transient expression systems may also be used. Homologous recombination may also be used.

Transfection may be accomplished by a wide variety of means, as is known to those of ordinary skill in the art. Such methods include, but are not limited to, Agrobacterium-mediated transformation (e.g., Komari et al., 1998, Curr. Opin. Plant Biol., 1:161), particle bombardment mediated transformation (e.g., Finer et al., 1999, Curr. Top. Microbiol. Immunol., 240:59), protoplast electroporation (e.g., Bates, 1999, Methods Mol. Biol., 111:359), viral infection (e.g., Porta and Lomonossoff, 1996, Mol. Biotechnol. 5:209), microinjection, and liposome injection. Other exemplary delivery systems that can be used to facilitate uptake by a cell of the nucleic acid include calcium phosphate and other chemical mediators of intracellular transport, microinjection compositions, and homologous recombination compositions (e.g., for integrating a gene into a preselected location within the chromosome of the cell). Alternative methods may involve, for example, the use of liposomes, electroporation, or chemicals that increase free (or “naked”) DNA uptake, transformation using viruses or pollen and the use of microprojection. Standard molecular biology techniques are common in the art (e.g., Sambrook et al., 1989, Molecular Cloning: A Laboratory Manual, 2nd ed., Cold Spring Harbor Laboratory Press, New York).

One of skill in the art will be able to select an appropriate vector for introducing the encoding nucleic acid sequence in a relatively intact state. Thus, any vector which will produce a host cell, e.g., plant protoplast, carrying the introduced encoding nucleic acid should be sufficient. The selection of the vector, or whether to use a vector, is typically guided by the method of transformation selected.

The transformation of plants cells in accordance with the invention may be carried out in essentially any of the various ways known to those skilled in the art of plant molecular biology. (See, for example, Methods of Enzymology, Vol. 153, 1987, Wu and Grossman, Eds., Academic Press, incorporated herein by reference).

Plant cells can comprise two or more nucleotide sequence constructs. Any means for producing a plant cell, e.g., protoplast, comprising the nucleotide sequence constructs described herein are encompassed by the present invention. For example, a nucleotide sequence encoding the modulator can be used to transform a plant cell at the same time as the nucleotide sequence encoding the precursor RNA. The nucleotide sequence encoding the precursor mRNA can be introduced into a plant cell that has already been transformed with the modulator nucleotide sequence. Likewise, viral vectors may be used to express gene products by various methods generally known in the art. Suitable plant viral vectors for expressing genes should be self-replicating, capable of systemic infection in a host, and stable. Additionally, the viruses should be capable of containing the nucleic acid sequences that are foreign to the native virus forming the vector.

Homologous recombination may be used as a method of gene inactivation.

The particular choice of a transformation technology will be determined by its efficiency to transform certain plant species as well as the experience and preference of the person practicing the invention with a particular methodology of choice. It will be apparent to the skilled person that the particular choice of a transformation system to introduce nucleic acid into plant cells is not essential to or a limitation of the invention, nor is the choice of technique for plant regeneration.

Agrobacterium.

The nucleic acid sequences utilized in the present invention can be introduced into plant cells using Ti plasmids of Agrobacterium tumefaciens (A. tumefaciens), root-inducing (Ri) plasmids of Agrobacterium rhizogenes (A. rhizogenes), and plant virus vectors. For reviews of such techniques see, for example, Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9, and Horsch et al., 1985, Science, 227:1229.

In using an A. tumefaciens culture as a transformation vehicle, it is most advantageous to use a non-oncogenic strain of Agrobacterium as the vector carrier so that normal non-oncogenic differentiation of the transformed tissues is possible. It is also preferred that the Agrobacterium harbor a binary Ti plasmid system. Such a binary system comprises 1) a first Ti plasmid having a virulence region essential for the introduction of transfer DNA (T-DNA) into plants, and 2) a chimeric plasmid. The chimeric plasmid contains at least one border region of the T-DNA region of a wild-type Ti plasmid flanking the nucleic acid to be transferred. Binary Ti plasmid systems have been shown effective in the transformation of plant cells (De Framond, Biotechnology, 1983, 1:262; Hoekema et al., 1983, Nature, 303:179). Such a binary system is preferred because it does not require integration into the Ti plasmid of A. tumefaciens, which is an older methodology.

In some embodiments, a disarmed Ti-plasmid vector carried by Agrobacterium exploits its natural gene transferability (EP-A-270355, EP-A-01 16718, Townsend et al., 1984, NAR, 12:8711, U.S. Pat. No. 5,563,055).

Methods involving the use of Agrobacterium in transformation according to the present invention include, but are not limited to: 1) co-cultivation of Agrobacterium with cultured isolated protoplasts; 2) transformation of plant cells or tissues with Agrobacterium; or 3) transformation of seeds, apices or meristems with Agrobacterium.

In addition, gene transfer can be accomplished by in planta transformation by Agrobacterium, as described by Bechtold et al., (C.R. Acad. Sci. Paris, 1993, 316:1194). This approach is based on the vacuum infiltration of a suspension of Agrobacterium cells.

In certain embodiments, nucleic acid molecue is introduced into plant cells by infecting such plant cells, an explant, a meristem or a seed, with transformed A. tumefaciens as described above. Under appropriate conditions known in the art, the transformed plant cells are grown to form shoots, roots, and develop further into plants.

Other methods described herein, such as microprojectile bombardment, electroporation and direct DNA uptake can be used where Agrobacterium is inefficient or ineffective. Alternatively, a combination of different techniques may be employed to enhance the efficiency of the transformation process, e.g., bombardment with Agrobacterium-coated microparticles (EP-A-486234) or microprojectile bombardment to induce wounding followed by co-cultivation with Agrobacterium (EP-A-486233).

CaMV.

In some embodiments, cauliflower mosaic virus (CaMV) is used as a vector for introducing a desired nucleic acid into plant cells (U.S. Pat. No. 4,407,956). CaMV viral DNA genome can be inserted into a parent bacterial plasmid creating a recombinant DNA molecule which can be propagated in bacteria. After cloning, the recombinant plasmid again can be cloned and further modified by introduction of the desired nucleic acid sequence. The modified viral portion of the recombinant plasmid can then be excised from the parent bacterial plasmid, and used to inoculate the plant cells or plants.

Mechanical and Chemical Means.

In some embodiments, a nucleic acid molecule of the invention is introduced into a plant cell using mechanical or chemical means. Exemplary mechanical and chemical means are provided below.

As used herein, the term “contacting” refers to any means of introducing a nucleic acid molecule into a plant cell, including chemical and physical means as described above. Preferably, contacting refers to introducing the nucleic acid or vector containing the nucleic acid into plant cells (including an explant, a meristem or a seed), via A. tumefaciens transformed with the nucleic acid molecule.

Microinjection.

In one embodiment, the nucleic acid molecule can be mechanically transferred into the plant cell by microinjection using a micropipette. See, e.g., WO 92/09696, WO 94/00583, EP 331083, EP 175966, Green et al., 1987, Plant Tissue and Cell Culture, Academic Press, Crossway et al., 1986, Biotechniques 4:320-334.

PEG.

In other embodiment, the nucleic acid can also be transferred into the plant cell by using polyethylene glycol (PEG) which forms a precipitation complex with genetic material that is taken up by the cell.

Electroporation.

Electroporation can be used, in another set of embodiments, to deliver a nucleic acid to the cell (see, e.g., Fromm et al., 1985, PNA5, 82:5824). “Electroporation,” as used herein, is the application of electricity to a cell, such as a plant protoplast, in such a way as to cause delivery of a nucleic acid into the cell without killing the cell. Typically, electroporation includes the application of one or more electrical voltage “pulses” having relatively short durations (usually less than 1 second, and often on the scale of milliseconds or microseconds) to a media containing the cells. The electrical pulses typically facilitate the non-lethal transport of extracellular nucleic acids into the cells. The exact electroporation protocols (such as the number of pulses, duration of pulses, pulse waveforms, etc.), will depend on factors such as the cell type, the cell media, the number of cells, the substance(s) to be delivered, etc., and can be determined by those of ordinary skill in the art. Electroporation is discussed in greater detail in, e.g., EP 290395, WO 8706614, Riggs et al., 1986, Proc. Natl. Acad. Sci. USA 83:5602-5606; D'Halluin et al., 1992, Plant Cell 4:1495-1505). Other forms of direct DNA uptake can also be used in the methods provided herein, such as those discussed in, e.g., DE 4005152, WO 9012096, U.S. Pat. No. 4,684,611, Paszkowski et al., 1984, EMBO J. 3:2717-2722.

Ballistic and Particle Bombardment.

Another method for introducing a nucleic acid molecule is high velocity ballistic penetration by small particles with the nucleic acid to be introduced contained either within the matrix of such particles, or on the surface thereof (Klein et al., 1987, Nature 327:70). Genetic material can be introduced into a cell using particle gun (“gene gun”) technology, also called microprojectile or microparticle bombardment. In this method, small, high-density particles (microprojectiles) are accelerated to high velocity in conjunction with a larger, powder-fired macroprojectile in a particle gun apparatus. The microprojectiles have sufficient momentum to penetrate cell walls and membranes, and can carry RNA or other nucleic acids into the interiors of bombarded cells. It has been demonstrated that such microprojectiles can enter cells without causing death of the cells, and that they can effectively deliver foreign genetic material into intact tissue. Bombardment transformation methods are also described in Sanford et al. (Techniques 3:3-16, 1991) and Klein et al. (Bio/Techniques 10:286, 1992). Although, typically only a single introduction of a new nucleic acid sequence(s) is required, this method particularly provides for multiple introductions.

Particle or microprojectile bombardment are discussed in greater detail in, e.g., the following references: U.S. Pat. No. 5,100,792, EP-A-444882, EP-A-434616; Sanford et al., U.S. Pat. No. 4,945,050; Tomes et al., 1995, “Direct DNA Transfer into Intact Plant Cells via Microprojectile Bombardment,” in Plant Cell, Tissue, and Organ Culture: Fundamental Methods, ed. Gamborg and Phillips (Springer-Verlag, Berlin); and McCabe et al., 1988, Biotechnology 6:923-926.

Colloidal Dispersion.

In other embodiments, a colloidal dispersion system may be used to facilitate delivery of a nucleic acid into the cell. As used herein, a “colloidal dispersion system” refers to a natural or synthetic molecule, other than those derived from bacteriological or viral sources, capable of delivering to and releasing the nucleic acid to the cell. Colloidal dispersion systems include, but are not limited to, macromolecular complexes, beads, and lipid-based systems including oil-in-water emulsions, micelles, mixed micelles, and liposomes. One example of a colloidal dispersion system is a liposome. Liposomes are artificial membrane vessels. It has been shown that large unilamellar vessels (“LUV”), which-range in size from 0.2 to 4.0 microns, can encapsulate large macromolecules within the aqueous interior and these macromolecules can be delivered to cells in a biologically active form (e.g., Fraley et al., 1981, Trends Biochem. Sci., 6:77).

Lipids.

Lipid formulations for the transfection and/or intracellular delivery of nucleic acids are commercially available, for instance, from QIAGEN, for example as EFFECTENE® (a non-liposomal lipid with a special DNA condensing enhancer) and SUPER-FECTO (a novel acting dendrimeric technology) as well as Gibco BRL, for example, as LIPOFECTINO and LIPOFECTACE®, which are formed of cationic lipids such as N-[1-(2,3-dioleyloxy)-propyl]-N,N,N-trimethylammonium chloride (“DOTMA”) and dimethyl dioctadecylammonium bromide (“DDAB”). Liposomes are well known in the art and have been widely described in the literature, for example, in Gregoriadis, G., 1985, Trends in Biotechnology 3:235-241; Freeman et al., 1984, Plant Cell Physiol. 29:1353).

Other Methods.

In addition to the above, other physical methods for the transformation of plant cells are reviewed in the following and can be used in the methods provided herein. Oard, 1991, Biotech. Adv. 9:1-11. See generally, Weissinger et al., 1988, sAnn. Rev. Genet. 22:421-477; Sanford et al., 1987, Particulate Science and Technology 5:27-37; Christou et al., 1988, Plant Physiol. 87:671-674; McCabe et al., 1988, Bio/Technology 6:923-926; Finer and McMullen, 1991, In vitro Cell Dev. Biol. 27P:175-182; Singh et al., 1998, Theor. Appl. Genet. 96:319-324; Datta et al., 1990, Biotechnology 8:736-740; Klein et al., 1988, Proc. Natl. Acad. Sci. USA 85:4305-4309; Klein et al., 1988, Biotechnology 6:559-563; Tomes, U.S. Pat. No. 5,240,855; Buising et al., U.S. Pat. Nos. 5,322,783 and 5,324,646; Klein et al., 1988, Plant Physiol. 91:440-444; Fromm et al., 1990, Biotechnology 8:833-839; Hooykaas-Van Slogteren et al., 1984, Nature (London) 311:763-764; Bytebier et al., 1987, Proc. Natl. Acad. Sci. USA 84:5345-5349; De Wet et al., 1985, The Experimental Manipulation of Ovule Tissues, ed. Chapman et al. (Longman, N.Y.), pp. 197-209; Kaeppler et al., 1990, Plant Cell Reports 9:415-418 and Kaeppler et al., 1992, Theor. Appl. Genet. 84:560-566; Li et al., 1993, Plant Cell Reports 12:250-255 and Christou and Ford, 1995, Annals of Botany 75:407-413; Osjoda et al., 1996, Nature Biotechnology 14:745-750; all of which are herein incorporated by reference.

5.2.3. Nucleic Acid Constructs

The nucleic acid molecules of the invention may be provided in nucleotide sequence constructs or expression cassettes for expression in the plant cell of interest. The cassette will include 5′ and 3′ regulatory sequences operably linked to an encoding nucleotide sequence of the invention.

The expression cassette may additionally contain at least one additional gene to be co-transformed into the organism. Alternatively, the additional gene(s) can be provided on multiple expression cassettes.

In certain embodiments, an expression cassette can be used with a plurality of restriction sites for insertion of the sequences of the invention to be under the transcriptional regulation of the regulatory regions. The expression cassette can additionally contain selectable marker genes (see below).

The expression cassette will generally include in the 5′-3′ direction of transcription, a transcriptional and translational initiation region, a DNA sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. By “foreign” is intended that the transcriptional initiation region is not found in the native plant into which the transcriptional initiation region is introduced. As used herein, a chimeric gene comprises a coding sequence operably linked to a transcription initiation region that is heterologous to the coding sequence.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al., 1991, Mol. Gen. Genet. 262:141-144; Proudfoot, 1991, Cell 64:671-674; Sanfacon et al., 1991, Genes Dev. 5:141-149; Mogen et al., 1990, Plant Cell 2:1261-1272; Munroe et al., 1990, Gene 91:151-158; Ballas et al., 1989, Nucleic Acids Res. 17:7891-7903; and Joshi et al., 1987, Nucleic Acid Res. 15:9627-9639.

In some embodiments, a nucleic acid can be delivered to the cell in a vector. As used herein, a “vector” is any vehicle capable of facilitating the transfer of the nucleic acid to the cell such that the nucleic acid can be processed and/or expressed in the cell. The vector may transport the nucleic acid to the cells with reduced degradation, relative to the extent of degradation that would result in the absence of the vector. The vector optionally includes gene expression sequences or other components (such as promoters and other regulatory elements) able to enhance expression of the nucleic acid within the cell. The invention also encompasses the cells transfected with these vectors, including those cells previously described.

To commence a transformation process in certain embodiments, it is first necessary to construct a suitable vector and properly introduce it into the plant cell. Vector(s) employed in the present invention for transformation of a plant cell include an encoding nucleic acid sequence operably associated with a promoter, such as a leaf-specific promoter. Details of the construction of vectors utilized herein are known to those skilled in the art of plant genetic engineering.

In general, vectors useful in the invention include, but are not limited to, plasmids, phagemids, viruses, other vehicles derived from viral or bacterial sources that have been manipulated by the insertion or incorporation of the nucleotide sequences (or precursor nucleotide sequences) of the invention. Viral vectors useful in certain embodiments include, but are not limited to, nucleic acid sequences from the following viruses: retroviruses; adenovirus, or other adeno-associated viruses; mosaic viruses such as tobamoviruses; potyviruses, nepoviruses, and RNA viruses such as retroviruses. One can readily employ other vectors not named but known to the art. Some viral vectors can be based on non-cytopathic eukaryotic viruses in which non-essential genes have been replaced with the nucleotide sequence of interest. Non-cytopathic viruses include retroviruses, the life cycle of which involves reverse transcription of genomic viral RNA into DNA with subsequent proviral integration into host cellular DNA.

Genetically altered retroviral expression vectors can have general utility for the high-efficiency transduction of nucleic acids. Standard protocols for producing replication-deficient retroviruses (including the steps of incorporation of exogenous genetic material into a plasmid, transfection of a packaging cell lined with plasmid, production of recombinant retroviruses by the packaging cell line, collection of viral particles from tissue culture media, and infection of the cells with viral particles) are well known to those of ordinary skill in the art. Examples of standard protocols can be found in Kriegler, M., 1990, Gene Transfer and Expression, A Laboratory Manual, W.H. Freeman Co., New York, or Murry, E. J. Ed., 1991, Methods in Molecular Biology, Vol. 7, Humana Press, Inc., Cliffton, N.J.

Another-example of a virus for certain applications is the adeno-associated virus, which is a double-stranded DNA virus. The adeno-associated virus can be engineered to be replication-deficient and is capable of infecting a wide range of-cell types and species. The adeno-associated virus further has advantages, such as heat and lipid solvent stability; high transduction frequencies in cells of diverse lineages; and/or lack of superinfection inhibition, which may allow multiple series of transductions.

Another vector suitable for use with the method provided herein is a plasmid vector. Plasmid vectors, have been extensively described in the art and are well-known to those of skill in the art. See, e.g., Sambrook et al., 1989, Molecular Cloning: A Laboratory Manual, Second Edition, Cold Spring Harbor Laboratory Press. These plasmids may have a promoter compatible with the host cell, and the plasmids can express a peptide from a gene operatively encoded within the plasmid. Some commonly used plasmids include pBR322, pUC18, pUC19, pRC/CMV, SV40, and pBlueScript. Other plasmids are well-known to those of ordinary skill in the art. Additionally, plasmids may be custom-designed, for example, using restriction enzymes and ligation reactions, to remove and add specific fragments of DNA or other nucleic acids, as necessary. The present invention also includes vectors for producing nucleic acids or precursor nucleic acids containing a desired nucleotide sequence (which can, for instance, then be cleaved or otherwise processed within the cell to produce a precursor miRNA). These vectors may include a sequence encoding a nucleic acid and an in vivo expression element, as further described below. In some cases, the in vivo expression element includes at least one promoter.

Where appropriate, the gene(s) for enhanced expression may be optimized for expression in the transformed plant. That is, the genes can be synthesized using plant-preferred codons corresponding to the plant of interest. Methods are available in the art for synthesizing plant-preferred genes. See, for example, U.S. Pat. Nos. 5,380,831, and 5,436,391, and Murray et al., 1989, Nucleic Acids Res. 17:477-498.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When desired, the sequence is modified to avoid predicted hairpin secondary mRNA structures. However, it is recognized that in the case of nucleotide sequences encoding the miRNA precursors, one or more hairpin and other secondary structures may be desired for proper processing of the precursor into an mature miRNA and/or for the functional activity of the miRNA in gene silencing.

The expression cassettes can additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al., 1989, PNAS USA 86:6126-6130); potyvirus leaders, for example, TEV leader (Tobacco Etch Virus) (Allison et al., 1986); MDMV leader (Maize Dwarf Mosaic Virus); Virology 154:9-20), and human immunoglobulin heavy-chain binding protein (BiP), (Macejak et al., 1991, Nature 353:90-94); untranslated leader from the coat protein miRNA of alfalfa mosaic virus (AMV RNA 4) (Jobling et al., 1987, Nature 325:622-625); tobacco mosaic virus leader (TMV) (Gallie et al., 1989, Molecular Biology of RNA, ed. Cech (Liss, New York), pp. 237-256); and maize chlorotic mottle virus leader (MCMV) (Lommel et al., 1991, Virology 81:382-385). See also, Della-Cioppa et al., 1987, Plant Physiol. 84:965-968.

In preparing the expression cassette, the various DNA fragments can be manipulated, so as to provide for the DNA sequences in the proper orientation and, as appropriate, in the proper reading frame. Toward this end, adapters or linkers can be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

5.2.4. Host Cells

Provided herein are host cells that contain a vector, e.g., a DNA plasmid and support the replication and/or expression of the vector. Host cells may be prokaryotic cells such as E. coli, or eukaryotic cells such as yeast, plant, insect, amphibian, or mammalian cells. In some embodiments, host cells are monocotyledonous or dicotyledonous plant cells. In other embodiments monocotyledonous host cell is a maize host cell. In certain embodiments, the host cell utilized in the methods of the present invention are transiently transfected with the nucleic acid molecules of the invention.

In preferred embodiments, the host cell utilized in the methods of the present invention is a plant protoplast. Plant protoplasts are plant cells that had their entire plant cell wall enzymatically removed prior to the introduction of the molecule of interest. The complete removal of the cell wall disrupts the connection between cells producing a homogenous suspension of individualized cells which allows more uniform and large scale transfection experiments. This comprises, but is not restricted to protoplast fusion, electroporation, liposome-mediated transfection, and polyethylene glycol-mediated transfection. Protoplast preparation is therefore a very reliable and inexpensive method to produce millions of cells.

In particular embodiments, the plant protoplast is derived from one of the following genuses: Acorus, Aegilops, Allium, Amborella, Antirrhinum, Apium, Arabidopsis, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia. In some embodiments, the host cell is derived from a genus that is different from the genus from which the transcription factor is derived from. For example, the host cell is a plant protoplast derived from the genus Arabidopsis and the transcription factor is derived from the genus Zea.

Also provided herein are plant cells having the nucleotide sequence constructs of the invention. A further aspect of the present invention provides a method of making such a plant cell involving introduction of a vector including the construct into a plant cell. For integration of the construct into the plant genome, such introduction will be followed by recombination between the vector and the plant cell genome to introduce the sequence of nucleotides into the genome. RNA encoded by the introduced nucleic acid construct may then be transcribed in the cell and descendants thereof, including cells in plants regenerated from transformed material. A gene stably incorporated into the genome of a plant is passed from generation to generation to descendants of the plant, so such descendants should show the desired phenotype.

Optionally, germ line cells may be used in the methods described herein rather than, or in addition to, somatic cells. The term “germ line cells” refers to cells in the plant organism which can trace their eventual cell lineage to either the male or female reproductive cell of the plant. Other cells, referred to as “somatic cells” are cells which give rise to leaves, roots and vascular elements which, although important to the plant, do not directly give rise to gamete cells. Somatic cells, however, also may be used. With regard to callus and suspension cells which have somatic embryogenesis, many or most of the cells in the culture have the potential capacity to give rise to an adult plant. If the plant originates from single cells or a small number of cells from the embryogenic callus or suspension culture, the cells in the callus and suspension can therefore be referred to as germ cells. In the case of immature embryos which are prepared for treatment by the methods described herein, certain cells in the apical meristem region of the plant have been shown to produce a cell lineage which eventually gives rise to the female and male reproductive organs. With many or most species, the apical meristem is generally regarded as giving rise to the lineage that eventually will give rise to the gamete cells. An example of a non-gamete cell in an embryo would be the first leaf primordia in corn which is destined to give rise only to the first leaf and none of the reproductive structures.

5.2.5. Promoters and Other Regulatory Sequences

In the broad method of the invention, the nucleic acid molecule of the invention is operably linked with a promoter. It may be desirable to introduce more than one copy of a polynucleotide into a plant cell for enhanced expression.

In general, promoters are found positioned 5′ (upstream) of the genes that they control. Thus, in the construction of promoter gene combinations, the promoter is preferably positioned upstream of the gene and at a distance from the transcription start site that approximates the distance between the promoter and the gene it controls in the natural setting. As is known in the art, some variation in this distance can be tolerated without loss of promoter function. Similarly, the preferred positioning of a regulatory element, such as an enhancer, with respect to a heterologous gene placed under its control reflects its natural position relative to the structural gene it naturally regulates.

Thus, the nucleic acid, in one embodiment, is operably linked to a gene expression sequence, which directs the expression of the nucleic acid within the cell. A “gene expression sequence,” as used herein, is any regulatory nucleotide sequence, such as a promoter sequence or promoter-enhancer combination, which facilitates the efficient transcription and translation of the nucleotide sequence to which it is operably linked. The gene expression sequence may, for example, be a eukaryotic promoter or a viral promoter, such as a constitutive or inducible promoter. Promoters and enhancers consist of short arrays of DNA sequences that interact specifically with cellular proteins involved in transcription, for instance, as discussed in Maniatis et al., 1987, Science 236:1237. Promoter and enhancer elements have been isolated from a variety of eukaryotic sources including genes in plant, yeast, insect and mammalian cells and viruses (analogous control elements, i.e., promoters, are also found in prokaryotes). In some embodiments, the nucleic acid is linked to a gene expression sequence which permits expression of the nucleic acid in a plant cell. A sequence which permits expression of the nucleic acid in a plant cell is one which is selectively active in the particular plant cell and thereby causes the expression of the nucleic acid in these cells. Those of ordinary skill in the art will be able to easily identify promoters that are capable of expressing a nucleic acid in a cell based on the type of plant cell.

A number of promoters can be used in the practice of the invention. The promoters can be selected based on the desired outcome. Generally, the nucleotide sequence and the modulator sequences can be combined with promoters of choice to alter gene expression if the target sequences in the tissue or organ of choice. Thus, the nucleotide sequence or modulator nucleotide sequence can be combined with constitutive, tissue-preferred, inducible, developmental, or other promoters for expression in plants depending upon the desired outcome.

The selection of a particular promoter and enhancer depends on what cell type is to be used and the mode of delivery. For example, a wide variety of promoters have been isolated from plants and animals, which are functional not only in the cellular source of the promoter, but also in numerous other plant species. There are also other promoters (e.g., viral and Ti-plasmid) which can be used. For example, these promoters include promoters from the Ti-plasmid, such as the octopine synthase promoter, the nopaline synthase promoter, the mannopine synthase promoter, and promoters from other open reading frames in the T-DNA, such as ORF7, etc. Promoters isolated from plant viruses include the 35S promoter from cauliflower mosaic virus. Promoters that have been isolated and reported for use in plants include ribulose-1,3-biphosphate carboxylase small subunit promoter, phaseolin promoter, etc. Thus, a variety of promoters and regulatory elements may be used in the expression vectors of the present invention.

Promoters useful in the compositions and methods provided herein include both natural constitutive and inducible promoters as well as engineered promoters. The CaMV promoters are examples of constitutive promoters. Other constitutive mammalian promoters include, but are not limited to, polymerase promoters as well as the promoters for the following genes: hypoxanthine phosphoribosyl transferase (“HPTR”), adenosine deaminase, pyruvate kinase, and alpha-actin.

Promoters useful as expression elements of the invention also include inducible promoters. Inducible promoters are expressed in the presence of an inducing agent. For example, a metallothionein promoter can be induced to promote transcription in the presence of certain metal ions. Other inducible promoters are known to those of ordinary skill in the art. The in vivo expression element can include, as necessary, 5′ non-transcribing and 5′ non-translating sequences involved with the initiation of transcription, and can optionally include enhancer sequences or upstream activator sequences.

For example, in some embodiments an inducible promoter is used to allow control of nucleic acid expression through the presentation of external stimuli (e.g., environmentally inducible promoters), as discussed below. Thus, the timing and amount of nucleic acid expression can be controlled in some cases. Non-limiting examples of expression systems, promoters, inducible promoters, environmentally inducible promoters, and enhancers are well known to those of ordinary skill in the art. Examples include those described in International Patent Application Publications WO 00/12714, WO 00/11175, WO 00/12713, WO 00/03012, WO 00/03017, WO 00/01832, WO 99/50428, WO 99/46976 and U.S. Pat. Nos. 6,028,250, 5,959,176, 5,907,086, 5,898,096, 5,824,857, 5,744,334, 5,689,044, and 5,612,472. A general descriptions of plant expression vectors and reporter genes can also be found in Gruber et al., 1993, “Vectors for Plant Transformation,” in Methods in Plant Molecular Biology & Biotechnology, Glich et al., Eds., p. 89-119, CRC Press.

For plant expression vectors, viral promoters that can be used in certain embodiments include the 35S RNA and 19S RNA promoters of CaMV (Brisson et al., Nature, 1984, 310:511; Odell et al., Nature, 1985, 313:810); the full-length transcript promoter from Figwort Mosaic Virus (FMV) (Gowda et al., 1989, J. Cell Biochem., 13D: 301) and the coat protein promoter to TMV (Takamatsu et al., 1987, EMBO J. 6:307). Alternatively, plant promoters such as the light-inducible promoter from the small subunit of ribulose bis-phosphate carboxylase (ssRUBISCO) (Coruzzi et al., 1984, EMBO J., 3:1671; Broglie et al., 1984, Science, 224:838); mannopine synthase promoter (Velten et al., 1984, EMBO J., 3:2723) nopaline synthase (NOS) and octopine synthase (OCS) promoters (carried on tumor-inducing plasmids of Agrobacterium tumefaciens) or heat shock promoters, e.g., soybean hsp17.5-E or hsp17.3-B (Gurley et al., 1986, Mol. Cell. Biol., 6:559; Severin et al., 1990, Plant Mol. Biol., 15:827) may be used. Exemplary viral promoters which function constitutively in eukaryotic cells include, for example, promoters from the simian virus, papilloma virus, adenovirus, human immunodeficiency virus, Rous sarcoma virus, cytomegalovirus, the long terminal repeats of Moloney leukemia virus and other retroviruses, and the thymidine kinase promoter of herpes simplex virus. Other constitutive promoters are known to those of ordinary skill in the art.

To be most useful, an inducible promoter should 1) provide low expression in the absence of the inducer; 2) provide high expression in the presence of the inducer; 3) use an induction scheme that does not interfere with the normal physiology of the plant; and 4) have no effect on the expression of other genes. Examples of inducible promoters useful in plants include those induced by chemical means, such as the yeast metallothionein promoter which is activated by copper ions (Mett et al., Proc. Natl. Acad. Sci., U.S.A., 90:4567, 1993); In2-1 and In2-2 regulator sequences which are activated by substituted benzenesulfonamides, e.g., herbicide safeners (Hershey et al., Plant Mol. Biol., 17:679, 1991); and the GRE regulatory sequences which are induced by glucocorticoids (Schena et al., Proc. Natl. Acad. Sci., U.S.A., 88:10421, 1991). Other promoters, both constitutive and inducible will be known to those of skill in the art.

A number of inducible promoters are known in the art. For resistance genes, a pathogen-inducible promoter can be utilized. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al., 1983, Neth. J. Plant Pathol. 89:245-254; Uknes et al., 1992, Plant Cell 4:645-656; and Van Loon, 1985, Plant Mol. Virol. 4:111-116. Of particular interest are promoters that are expressed locally at or near the site of pathogen infection. See, for example, Marineau et al., 1987, Plant Mol. Biol. 9:335-342; Matton et al., 1989, Molecular Plant-Microbe Interactions 2:325-331; Somsisch et al., 1986, Proc. Natl. Acad. Sci. USA 83:2427-2430; Somsisch et al., 1988, Mol. Gen. Genet. 2:93-98; and Yang, 1996, Proc. Natl. Acad. Sci. USA 93:14972-14977. See also, Chen et al., 1996, Plant J. 10:955-966; Zhang et al., 1994, Proc. Natl. Acad. Sci. USA 91:2507-2511; Warner et al., 1993, Plant J. 3:191-201; Siebertz et al., 1989, Plant Cell 1:961-968; U.S. Pat. No. 5,750,386; Cordero et al., 1992, Physiol. Mol. Plant Path. 41:189-200; and the references cited therein.

Additionally, as pathogens find entry into plants through wounds or insect damage, a wound-inducible promoter may be used in the DNA constructs of the invention. Such wound-inducible promoters include potato proteinase inhibitor (pin II) gene (Ryan, 1990, Ann. Rev. Phytopath. 28:425-449; Duan et al., 1996, Nature Biotechnology 14:494-498); wun1 and wun2, U.S. Pat. No. 5,428,148; win1 and win2 (Stanford et al., 1989, Mol. Gen. Genet. 215:200-208); systemin (McGurl et al., 1992, Science 225:1570-1573); WIPI (Rohmeier et al., 1993, Plant Mol. Biol. 22:783-792; Eckelkamp et al., 1993, FEBS Letters 323:73-76); MPI gene (Corderok et al., 1994, Plant J. 6(2):141-150); and the like. Such references are herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1 a promoter, which is activated by salicylic acid. Other chemical-regulated promoters of interest include steroid-responsive promoters (see, for example, the glucocorticoid-inducible promoter in Schena et al., 1991, Proc. Natl. Acad. Sci. USA 88:10421-10425 and McNellis et al., 1998, Plant J. 14(2):247-257) and tetramiR167e-inducible and tetramiR167e-repressible promoters (see, for example, Gatz et al., 1991, Mol. Gen. Genet. 227:229-237, and U.S. Pat. Nos. 5,814,618 and 5,789,156), herein incorporated by reference.

Where enhanced expression in particular tissues is desired, tissue-preferred promoters can be utilized. Tissue-preferred promoters include those described by Yamamoto et al., 1997, Plant J. 12(2):255-265; Kawamata et al., 1997, Plant Cell Physiol. 38(7):792-803; Hansen et al., 1997, Mol. Gen Genet. 254(3):337-343; Russell et al., 1997, Transgenic Res. 6(2):157-168; Rinehart et al., 1996, Plant Physiol. 112(3):1331-1341; Van Camp et al., 1996, Plant Physiol. 112(2):525-535; Canevascini et al., 1996, Plant Physiol. 12(2):513-524; Yamamoto et al., 1994, Plant Cell Physiol. 35(5):773-778; Lam, 1994, Results Probl. Cell Differ. 20:181-196; Orozco et al., 1993, Plant Mol. Biol. 23(6): 1129-1138; Matsuoka et al., 1993, Proc Natl. Acad. Sci. USA 90(20):9586-9590; and Guevara-Garcia et al., 1993, Plant J 4(3):495-505.

The particular promoter selected should be capable of causing sufficient expression to result in the production of an effective amount of structural gene product in the plant cell to cause upregulation of genes as compared to wild type. The promoters used in the vector constructs of the present invention may be modified, if desired, to affect their control characteristics. In certain embodiments, chimeric promoters can be used.

There are promoters known which limit expression to particular plant parts or in response to particular stimuli. One skilled in the art will know of many such plant part-specific promoters which would be useful in the present invention. In certain embodiments, to provide pericycle-specific expression, any of a number of promoters from genes in Arabidopsis can be used. In some embodiments, the promoter from one (or more) of the following genes may be used: (i) At1g11080, (ii) At3g60160, (iii) At1g24575, (iv) At3g45160, or (v) At1g23130. In specific embodiments, (vi) promoter elements from the GFP-marker line used in Gifford et al. (in preparation) will be used (see also, Bonke et al., 2003, Nature 426, 181-6; Tian et al., 2004, Plant Physiol 135, 25-38). Several of the predicted genes have a number of potential orthologs in rice and poplar and thus are predicted that they will be applicable for use in crop species; (i) Os04g44410, Os10g39560, Os06g51370, Os02g42310, Os01g22980, Os05g06660, and Poptr1#568263, Poptr1#555534, Poptr1#365170; (ii) Os04g49900, Os04g49890, Os01g67580, and Poptr1#87573, Poptr1#80582, Poptr1#565079, Poptr1#99223.

Promoters used in the nucleic acid constructs of the present invention can be modified, if desired, to affect their control characteristics. For example, the CaMV 35S promoter may be ligated to the portion of the ssRUBISCO gene that represses the expression of ssRUBISCO in the absence of light, to create a promoter which is active in leaves but not in roots. The resulting chimeric promoter may be used as described herein. For purposes of this description, the phrase “CaMV 35S” promoter thus includes variations of CaMV 35S promoter, e.g., promoters derived by means of ligation with operator regions, random or controlled mutagenesis, etc. Furthermore, the promoters may be altered to contain multiple “enhancer sequences” to assist in elevating gene expression.

An efficient plant promoter that may be used in specific embodiments is an “overproducing” or “overexpressing” plant promoter. Overexpressing plant promoters that can be used in the compositions and methods provided herein include the promoter of the small sub-unit (“ss”) of the ribulose-1,5-biphosphate carboxylase from soybean (e.g., Berry-Lowe et al., 1982, J. Molecular & App. Genet., 1:483), and the promoter of the chorophyll a-b binding protein. These two promoters are known to be light-induced in eukaryotic plant cells. For example, see Cashmore, Genetic Engineering of plants: An Agricultural Perspective, p. 29-38; Coruzzi et al., 1983, J. Biol. Chem., 258:1399; and Dunsmuir et al., 1983, J. Molecular & App. Genet., 2:285.

The promoters and control elements of, e.g., SUCS (root nodules; broadbean; Kuster et al., 1993, Mol Plant Microbe Interact 6:507-14) for roots can be used in compositions and methods provided herein to confer tissue specificity.

In certain embodiment, two promoter elements can be used in combination, such as, for example, (i) an inducible element responsive to a treatment that can be provided to the plant prior to N-fertilizer treatment, and (ii) a plant tissue-specific expression element to drive expression in the specific tissue alone.

Any promoter of other expression element described herein or known in the art may be used either alone or in combination with any other promoter or other expression element described herein or known in the art. For example, promoter elements that confer tissue specific expression of a gene can be used with other promoter elements conferring constitutive or inducible expression.

5.2.6. Isolating Related Promoter Sequences

Promoter and promoter control elements that are related to those described in herein can also be used in the compositions and methods provided herein. Such related sequence can be isolated utilizing (a) nucleotide sequence identity; (b) coding sequence identity of related, orthologous genes; or (c) common function or gene products.

Relatives can include both naturally occurring promoters and non-natural promoter sequences. Non-natural related promoters include nucleotide substitutions, insertions or deletions of naturally-occurring promoter sequences that do not substantially affect transcription modulation activity. For example, the binding of relevant DNA binding proteins can still occur with the non-natural promoter sequences and promoter control elements of the present invention.

According to current knowledge, promoter sequences and promoter control elements exist as functionally important regions, such as protein binding sites, and spacer regions. These spacer regions are apparently required for proper positioning of the protein binding sites. Thus, nucleotide substitutions, insertions and deletions can be tolerated in these spacer regions to a certain degree without loss of function.

In contrast, less variation is permissible in the functionally important regions, since changes in the sequence can interfere with protein binding. Nonetheless, some variation in the functionally important regions is permissible so long as function is conserved.

The effects of substitutions, insertions and deletions to the promoter sequences or promoter control elements may be to increase or decrease the binding of relevant DNA binding proteins to modulate transcript levels of a polynucleotide to be transcribed. Effects may include tissue-specific or condition-specific modulation of transcript levels of the polypeptide to be transcribed. Polynucleotides representing changes to the nucleotide sequence of the DNA-protein contact region by insertion of additional nucleotides, changes to identity of relevant nucleotides, including use of chemically-modified bases, or deletion of one or more nucleotides are considered encompassed by the present invention.

Typically, related promoters exhibit at least 80% sequence identity, preferably at least 85%, more preferably at least 90%, and most preferably at least 95%, even more preferably, at least 96%, at least 97%, at least 98% or at least 99% sequence identity. Such sequence identity can be calculated by the algorithms and computers programs described above.

Usually, such sequence identity is exhibited in an alignment region that is at least 75% of the length of a sequence or corresponding full-length sequence of a promoter described herein; more usually at least 80%; more usually, at least 85%, more usually at least 90%, and most usually at least 95%, even more usually, at least 96%, at least 97%, at least 98% or at least 99% of the length of a sequence of a promoter described herein.

The percentage of the alignment length is calculated by counting the number of residues of the sequence in region of strongest alignment, e.g., a continuous region of the sequence that contains the greatest number of residues that are identical to the residues between two sequences that are being aligned. The number of residues in the region of strongest alignment is divided by the total residue length of a sequence of a promoter described herein. These related promoters may exhibit similar preferential transcription as those promoters described herein.

In certain embodiments, a promoter, such as a leaf-preferred or leaf-specific promoter, can be identified by sequence homology or sequence identity to any root specific promoter identified herein. In other embodiments, orthologous genes identified herein as leaf-specific genes (e.g., the same gene or different gene that if functionally equivalent) for a given species can be identified and the associated promoter can also be used in the compositions and methods provided herein. For example, using high, medium or low stringency conditions, standard promoter rules can be used to identify other useful promoters from orthologous genes for use in the compositions and methods provided herein. In specific embodiments, the orthologous gene is a gene expressed only or primarily in the root, such as pericycle cells.

Polynucleotides can be tested for activity by cloning the sequence into an appropriate vector, transforming plants with the construct and assaying for marker gene expression. Recombinant DNA constructs can be prepared, which comprise the polynucleotide sequences of the invention inserted into a vector suitable for transformation of plant cells. The construct can be made using standard recombinant DNA techniques (Sambrook et al., 1989) and can be introduced to the species of interest by Agrobacterium-mediated transformation or by other means of transformation as referenced below.

The vector backbone can be any of those typical in the art such as plasmids, viruses, artificial chromosomes, BACs, YACs and PACs and vectors of the sort described by (a) BAC: Shizuya et al., 1992, Proc. Natl. Acad. Sci. USA 89: 8794-8797; Hamilton et al., 1996, Proc. Natl. Acad. Sci. USA 93: 9975-9979; (b) YAC: Burke et al., 1987, Science 236:806-812; (c) PAC: Stemberg N. et al., 1990, Proc Natl Acad Sci USA. January; 87(1):103-7; (d) Bacteria-Yeast Shuttle Vectors: Bradshaw et al., 1995, Nucl Acids Res 23: 4850-4856; (e) Lambda Phage Vectors: Replacement Vector, e.g., Frischauf et al., 1983, J. Mol. Biol. 170: 827-842; or Insertion vector, e.g., Huynh et al., 1985, In: Glover N M (ed) DNA Cloning: A practical Approach, Vol. 1 Oxford: IRL Press; T-DNA gene fusion vectors: Walden et al., 1990, Mol Cell Biol 1: 175-194; and (g) Plasmid vectors: Sambrook et al., infra.

Typically, the construct comprises a vector containing a sequence of the present invention operationally linked to any marker gene. The polynucleotide was identified as a promoter by the expression of the marker gene. Although many marker genes can be used, Green Fluorescent Protein (GFP) is preferred. The vector may also comprise a marker gene that confers a selectable phenotype on plant cells. The marker may encode biocide resistance, particularly antibiotic resistance, such as resistance to kanamycin, G418, bleomycin, hygromycin, or herbicide resistance, such as resistance to chlorosulfuron or phosphinotricin (see below). Vectors can also include origins of replication, scaffold attachment regions (SARs), markers, homologous sequences, introns, etc.

5.2.7. Cell-Type Preferential Transcription

Specific promoters may be used in the compositions and methods provided herein. As used herein, “specific promoters” refers to a subset of promoters that have a high preference for modulating transcript levels in a specific tissue or organ or cell and/or at a specific time during development of an organism. By “high preference” is meant at least 3-fold, preferably 5-fold, more preferably at least 10-fold still more preferably at least 20-fold, 50-fold or 100-fold increase in transcript levels under the specific condition over the transcription under any other reference condition considered. Typical examples of temporal and/or tissue or organ specific promoters of plant origin that can be used in the compositions and methods of the present invention, inlcude RCc2 and RCc3, promoters that direct root-specific gene transcription in rice (Xu et al., 1995, Plant Mol. Biol. 27:237 and TobRB27, a root-specific promoter from tobacco (Yamamoto et al., 1991, Plant Cell 3:371). Examples of tissue-specific promoters under developmental control include promoters that initiate transcription only in certain tissues or organs, such as roots

“Preferential transcription” is defined as transcription that occurs in a particular pattern of cell types or developmental times or in response to specific stimuli or combination thereof. Non-limitative examples of preferential transcription include: high transcript levels of a desired sequence in root tissues; detectable transcript levels of a desired sequence in certain cell types during embryogenesis; and low transcript levels of a desired sequence under drought conditions. Such preferential transcription can be determined by measuring initiation, rate, and/or levels of transcription.

Typically, promoter or control elements, which provide preferential transcription in cells, tissues, or organs of a root, produce transcript levels that are statistically significant as compared to other cells, organs or tissues. For preferential up-regulation of transcription, promoter and control elements produce transcript levels that are above background of the assay.

5.2.8. Selection and Identification of Transfected Host Cells

The method of the present invention comprises detecting host cells that express a selectable marker. In certain embodiments, the step of detecting host cells that express the selectable marker is performed by Fluorescence Activated Cell Sorting (FACS) in the methods of the present invention. Fluorescence activated cell sorting (FACS) is a well-known method for separating particles, including cells, based on the fluorescent properties of the particles (see, e.g., Kamarch, 1987, Methods Enzymol, 151:150-165). Laser excitation of fluorescent moieties in the individual particles results in a small electrical charge allowing electromagnetic separation of positive and negative particles from a mixture. In one embodiment, cell surface marker-specific antibodies or ligands are labeled with distinct fluorescent labels. Cells are processed through the cell sorter, allowing separation of cells based on their ability to bind to the antibodies used. FACS sorted particles may be directly deposited into individual wells of 96-well or 384-well plates to facilitate separation and cloning.

Also, desired plants may be obtained by engineering the disclosed gene constructs into a variety of plant cell types, including but not limited to, protoplasts, tissue culture cells, tissue and organ explants, pollens, embryos as well as whole plants. In an embodiment of the present invention, the engineered plant material is selected or screened for transformants (those that have incorporated or integrated the introduced gene construct(s)) following the approaches and methods described below. An isolated transformant may then be regenerated into a plant. Alternatively, the engineered plant material may be regenerated into a plant or plantlet before subjecting the derived plant or plantlet to selection or screening for the marker gene traits. Procedures for regenerating plants from plant cells, tissues or organs, either before or after selecting or screening for marker gene(s), are well known to those skilled in the art.

A transformed plant cell, callus, tissue or plant may be identified and isolated by selecting or screening the engineered plant material for traits encoded by the marker genes present on the transforming DNA. For instance, selection may be performed by growing the engineered plant material on media containing inhibitory amount of the antibiotic or herbicide to which the transforming gene construct confers resistance. Further, transformed plants and plant cells may also be identified by screening for the activities of any visible marker genes (e.g., the β-glucuronidase, luciferase, B or C1 genes) that may be present on the recombinant nucleic acid constructs of the present invention. Such selection and screening methodologies are well known to those skilled in the art.

Physical and biochemical methods also may be also to identify plant or plant cell transformants containing the gene constructs of the present invention. These methods include but are not limited to: 1) Southern analysis or PCR amplification for detecting and determining the structure of the recombinant DNA insert; 2) Northern blot, Si RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct; 4) protein gel electrophoresis, Western blot techniques, immunoprecipitation, or enzyme-linked immunoassays, where the gene construct products are proteins. Additional techniques, such as in situ hybridization, enzyme staining, and immunostaining, also may be used to detect the presence or expression of the recombinant construct in specific plant organs and tissues. The methods for doing all these assays are well known to those skilled in the art.

5.2.9. Plant Regeneration

Following transformation, a plant may be regenerated, e.g., from single cells, callus tissue or leaf discs, as is standard in the art. Almost any plant can be entirely regenerated from cells, tissues, and organs of the plant. Available techniques are reviewed in Vasil et al., 1984, in Cell Culture and Somatic Cell Genetics of Plants, Vols. I, II, and III, Laboratory Procedures and Their Applications (Academic Press); and Weissbach et al., 1989, Methods For Plant Mol. Biol.

The transformed plants may then be grown, and either pollinated with the same transformed strain or different strains, and the resulting hybrid having expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved.

Normally, a plant cell is regenerated to obtain a whole plant from the transformation process. The term “growing” or “regeneration” as used herein means growing a whole plant from a plant cell, a group of plant cells, a plant part (including seeds), or a plant piece (e.g., from a protoplast, callus, or tissue part).

Regeneration from protoplasts varies from species to species of plants, but generally a suspension of protoplasts is first made. In certain species, embryo formation can then be induced from the protoplast suspension. The culture media will generally contain various amino acids and hormones, necessary for growth and regeneration. Examples of hormones utilized include auxins and cytokinins Efficient regeneration will depend on the medium, on the genotype, and on the history of the culture. If these variables are controlled, regeneration is reproducible.

Regeneration also occurs from plant callus, explants, organs or parts. Transformation can be performed in the context of organ or plant part regeneration (see Methods in Enzymology, Vol. 118 and Klee et al., Annual Review of Plant Physiology, 38:467, 1987). Utilizing the leaf disk-transformation-regeneration method of Horsch et al., Science, 227:1229, 1985, disks are cultured on selective media, followed by shoot formation in about 2-4 weeks. Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature transgenic plants are propagated by utilizing cuttings or tissue culture techniques to produce multiple identical plants. Selection of desirable transgenics is made and new varieties are obtained and propagated vegetatively for commercial use.

In seed propagated crops, mature transgenic plants can be self crossed to produce a homozygous inbred plant. The resulting inbred plant produces seed containing the newly introduced foreign gene(s). These seeds can be grown to produce plants that would produce the selected phenotype, e.g., increased lateral root growth, uptake of nutrients, overall plant growth and/or vegetative or reproductive yields.

Parts obtained from the regenerated plant, such as flowers, seeds, leaves, branches, fruit, and the like are included in the invention, provided that these parts comprise cells comprising the isolated nucleic acid of the present invention. Progeny and variants, and mutants of the regenerated plants are also included within the scope of the invention, provided that these parts comprise the introduced nucleic acid sequences. Transgenic plants expressing the selectable marker can be screened for transmission of the nucleic acid of the present invention by, for example, standard immunoblot and DNA detection techniques. Transgenic lines are also typically evaluated on levels of expression of the heterologous nucleic acid. Expression at the RNA level can be determined initially to identify and quantitate expression-positive plants. Standard techniques for RNA analysis can be employed and include PCR amplification assays using oligonucleotide primers designed to amplify only the heterologous RNA templates and solution hybridization assays using heterologous nucleic acid-specific probes. The RNA-positive plants can then analyzed for protein expression by Western immunoblot analysis using the specifically reactive antibodies of the present invention. In addition, in situ hybridization and immunocytochemistry according to standard protocols can be done using heterologous nucleic acid specific polynucleotide probes and antibodies, respectively, to localize sites of expression within transgenic tissue. Generally, a number of transgenic lines are usually screened for the incorporated nucleic acid to identify and select plants with the most appropriate expression profiles.

A preferred embodiment is a transgenic plant that is homozygous for the added heterologous nucleic acid; i.e., a transgenic plant that contains two added nucleic acid sequences, one gene at the same locus on each chromosome of a chromosome pair. A homozygous transgenic plant can be obtained by sexually mating (selfing) a heterozygous transgenic plant that contains a single added heterologous nucleic acid, germinating some of the seed produced and analyzing the resulting plants produced for altered expression of a polynucleotide of the present invention relative to a control plant (i.e., native, non-transgenic). Back-crossing to a parental plant and out-crossing with a non-transgenic plant are also contemplated.

Transformed plant cells which are derived by any of the above transformation techniques can be cultured to regenerate a whole plant which possesses the transformed genotype. Such regeneration techniques often rely on manipulation of certain phytohormones in a tissue culture growth medium. For transformation and regeneration of maize see, Gordon-Kamm et al., 1990, The Plant Cell, 2:603-618.

Plants cells transformed with a plant expression vector can be regenerated, e.g., from single cells, callus tissue or leaf discs according to standard plant tissue culture techniques. It is well known in the art that various cells, tissues, and organs from almost any plant can be successfully cultured to regenerate an entire plant. Plant regeneration from cultured protoplasts is described in Evans et al., 1983, Protoplasts Isolation and Culture, Handbook of Plant Cell Culture, Macmillan Publishing Company, New York, pp. 124-176; and Binding, Regeneration of Plants, Plant Protoplasts, 1985, CRC Press, Boca Raton, pp. 21-73.

The regeneration of plants containing the foreign gene introduced by Agrobacterium from leaf explants can be achieved as described by Horsch et al., 1985, Science, 227:1229-1231. In this procedure, transformants are grown in the presence of a selection agent and in a medium that induces the regeneration of shoots in the plant species being transformed as described by Fraley et al., 1983, Proc. Natl. Acad. Sci. (U.S.A.), 80:4803. This procedure typically produces shoots within two to four weeks and these transformant shoots are then transferred to an appropriate root-inducing medium containing the selective agent and an antibiotic to prevent bacterial growth. Transgenic plants of the present invention may be fertile or sterile.

The regeneration of plants from either single plant protoplasts or various explants is well known in the art. See, for example, Methods for Plant Molecular Biology, A. Weissbach and H. Weissbach, eds., 1988, Academic Press, Inc., San Diego, Calif. This regeneration and growth process includes the steps of selection of transformant cells and shoots, rooting the transformant shoots and growth of the plantlets in soil. For maize cell culture and regeneration see generally, The Maize Handbook, Freeling and Walbot, Eds., 1994, Springer, New York 1994; Corn and Corn Improvement, 3rd edition, Sprague and Dudley Eds., 1988, American Society of Agronomy, Madison, Wis.

5.2.10. Plants

The present invention also provides a plant comprising a plant cell as disclosed. Transformed seeds and plant parts are also encompassed.

In addition to a plant, the present invention provides any clone of such a plant, seed, selfed or hybrid progeny and descendants, and any part of any of these, such as cuttings, seed. The invention provides any plant propagule, that is any part which may be used in reproduction or propagation, sexual or asexual, including cuttings, seed and so on. Also encompassed by the invention is a plant which is a sexually or asexually propagated off-spring, clone or descendant of such a plant, or any part or propagule of said plant, off-spring, clone or descendant. Plant extracts and derivatives are also provided.

Any species of woody, ornamental or decorative, crop or cereal, fruit or vegetable plant, and algae (e.g., Chlamydomonas reinhardtii) may be used in the compositions and methods provided herein. Non-limiting examples of plants include plants from the genus Arabidopsis or the genus Oryza. Other examples include plants from the genuses Acorus, Aegilops, Allium, Amborella, Antirrhinum, Apium, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Peryea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

Plants included in the invention are any plants amenable to transformation techniques, including gymnosperms and angiosperms, both monocotyledons and dicotyledons.

Examples of monocotyledonous angiosperms include, but are not limited to, asparagus, field and sweet corn, barley, wheat, rice, sorghum, onion, pearl millet, rye and oats and other cereal grains.

Examples of dicotyledonous angiosperms include, but are not limited to tomato, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers and various ornamentals.

Examples of woody species include poplar, pine, sequoia, cedar, oak, etc.

Still other examples of plants include, but are not limited to, wheat, cauliflower, tomato, tobacco, corn, petunia, trees, etc.

In certain embodiments, plants of the present invention are crop plants (for example, cereals and pulses, maize, wheat, potatoes, tapioca, rice, sorghum, millet, cassaya, barley, pea, and other root, tuber, or seed crops. Exemplary cereal crops used in the compositions and methods of the invention include, but are not limited to, any species of grass, or grain plant (e.g., barley, corn, oats, rice, wild rice, rye, wheat, millet, sorghum, triticale, etc.), non-grass plants (e.g., buckwheat flax, legumes or soybeans, etc.). Grain plants that provide seeds of interest include oil-seed plants and leguminous plants. Other seeds of interest include grain seeds, such as corn, wheat, barley, rice, sorghum, rye, etc. Oil seed plants include cotton, soybean, safflower, sunflower, Brassica, maize, alfalfa, palm, coconut, etc. Other important seed crops are oil-seed rape, sugar beet, maize, sunflower, soybean, and sorghum. Leguminous plants include beans and peas. Beans include guar, locust bean, fenugreek, soybean, garden beans, cowpea, mungbean, lima bean, fava bean, lentils, chickpea, etc.

Horticultural plants to which the present invention may be applied may include lettuce, endive, and vegetable brassicas including cabbage, broccoli, and cauliflower, and carnations and geraniums. The present invention may also be applied to tobacco, cucurbits, carrot, strawberry, sunflower, tomato, pepper, chrysanthemum, poplar, eucalyptus, and pine.

The present invention may be used for transformation of other plant species, including, but not limited to, corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum, Nicotiana benthamiana), potato (Solanum tuberosum), peanuts (Arachis hypogaea), cotton (Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats, barley, Arabidopsis spp., vegetables, ornamentals, and conifers.

5.2.11. Cultivation

Methods of cultivation of plants are well known in the art. For example, for the cultivation of wheat see Alcoz et al., 1993, Agronomy Journal 85:1198-1203; Rao and Dao, 1992, J. Am. Soc. Agronomy 84:1028-1032; Howard and Lessman, 1991, Agronomy Journal 83:208-211; for the cultivation of corn see Tollenear et al., 1993, Agronomy Journal 85:251-255; Straw et al., Tennessee Farm and Home Science: Progress Report, Spring 1993, 166:20-24; Miles, S. R., 1934, J. Am. Soc. Agronomy 26:129-137; Dara et al., 1992, J. Am. Soc. Agronomy 84:1006-1010; Binford et al., 1992, Agronomy Journal 84:53-59; for the cultivation of soybean see Chen et al., 1992, Canadian Journal of Plant Science 72:1049-1056; Wallace et al., 1990, Journal of Plant Nutrition 13:1523-1537; for the cultivation of rice see Oritani and Yoshida, 1984, Japanese Journal of Crop Science 53:204-212; for the cultivation of linseed see Diepenbrock and Porksen, 1992, Industrial Crops and Products 1:165-173; for the cultivation of tomato see Grubinger et al., 1993, Journal of the American Society for Horticultural Science 118:212-216; Cerne, M., 1990, Acta Horticulture 277:179-182; for the cultivation of pineapple see Magistad et al., 1932, J. Am. Soc. Agronomy 24:610-622; Asoegwu, S, N., 1988, Fertilizer Research 15:203-210; Asoegwu, S, N., 1987, Fruits 42:505-509; for the cultivation of lettuce see Richardson and Hardgrave, 1992, Journal of the Science of Food and Agriculture 59:345-349; for the cultivation of mint see Munsi, P. S., 1992, Acta Horticulturae 306:436-443; for the cultivation of camomile see Letchamo, W., 1992, Acta Horticulturae 306:375-384; for the cultivation of tobacco see Sisson et al., 1991, Crop Science 31:1615-1620; for the cultivation of potato see Porter and Sisson, 1991, American Potato Journal, 68:493-505; for the cultivation of brassica crops see Rahn et al., 1992, Conference “Proceedings, second congress of the European Society for Agronomy” Warwick Univ., p. 424-425; for the cultivation of banana see Hegde and Srinivas, 1991, Tropical Agriculture 68:331-334; Langenegger and Smith, 1988, Fruits 43:639-643; for the cultivation of strawberries see Human and Kotze, 1990, Communications in Soil Science and Plant Analysis 21:771-782; for the cultivation of songhum see Mahalle and Seth, 1989, Indian Journal of Agricultural Sciences 59:395-397; for the cultivation of plantain see Anjorin and Obigbesan, 1985, Conference “International Cooperation for Effective Plantain and Banana Research” Proceedings of the third meeting. Abidjan, Ivory Coast, p. 115-117; for the cultivation of sugar cane see Yadav, R. L., 1986, Fertiliser News 31:17-22; Yadav and Sharma, 1983, Indian Journal of Agricultural Sciences 53:38-43; for the cultivation of sugar beet see Draycott et al., 1983, Conference “Symposium Nitrogen and Sugar Beet” International Institute for Sugar Beet Research—Brussels Belgium, p. 293-303. See also Goh and Haynes, 1986, “Nitrogen and Agronomic Practice” in Mineral Nitrogen in the Plant-Soil System, Academic Press, Inc., Orlando, Fla., p. 379-468; Engelstad, O. P., 1985, Fertilizer Technology and Use, Third Edition, Soil Science Society of America, p. 633; Yadav and Sharmna, 1983, Indian Journal of Agricultural Sciences, 53:3-43.

5.2.12. Products of Transgenic Plants

Engineered plants exhibiting the desired physiological and/or agronomic changes can be used directly in agricultural production.

Thus, provided herein are products derived from the transgenic plants or methods of producing transgenic plants provided herein. In certain embodiments, the products are commercial products. Some non-limiting example include genetically engineered trees for e.g., the production of pulp, paper, paper products or lumber; tobacco, e.g., for the production of cigarettes, cigars, or chewing tobacco; crops, e.g., for the production of fruits, vegetables and other food, including grains, e.g., for the production of wheat, bread, flour, rice, corn; and canola, sunflower, e.g., for the production of oils or biofuels.

In certain embodiments, commercial products are derived from a genetically engineered (e.g., comprising overexpression of GLK1 in the vegetative tissues of the plant) species of woody, ornamental or decorative, crop or cereal, fruit or vegetable plant, and algae (e.g., Chlamydomonas reinhardtii), which may be used in the compositions and methods provided herein. Non-limiting examples of plants include plants from the genus Arabidopsis or the genus Oryza. Other examples include plants from the genuses Acorus, Aegilops, Allium, Amborella, Antirrhinum, Apium, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

In some embodiments, commercial products are derived from a genetically engineered gymnosperms and angiosperms, both monocotyledons and dicotyledons. Examples of monocotyledonous angiosperms include, but are not limited to, asparagus, field and sweet corn, barley, wheat, rice, sorghum, onion, pearl millet, rye and oats and other cereal grains. Examples of dicotyledonous angiosperms include, but are not limited to tomato, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers and various ornamentals.

In certain embodiments, commercial products are derived from a genetically engineered woody species, such as poplar, pine, sequoia, cedar, oak, etc.

In other embodiments, commercial products are derived from a genetically engineered plant including, but are not limited to, wheat, cauliflower, tomato, tobacco, corn, petunia, trees, etc.

In certain embodiments, commercial products are derived from a genetically engineered crop plants, for example, cereals and pulses, maize, wheat, potatoes, tapioca, rice, sorghum, millet, cassaya, barley, pea, and other root, tuber, or seed crops. In one embodiment, commercial products are derived from a genetically engineered cereal crops, including, but are not limited to, any species of grass, or grain plant (e.g., barley, corn, oats, rice, wild rice, rye, wheat, millet, sorghum, triticale, etc.), non-grass plants (e.g., buckwheat flax, legumes or soybeans, etc.). In another embodiments, commercial products are derived from a genetically engineered grain plants that provide seeds of interest, oil-seed plants and leguminous plants. In other embodiments, commercial products are derived from a genetically engineered grain seed plants, such as corn, wheat, barley, rice, sorghum, rye, etc. In yet other embodiments, commercial products are derived from a genetically engineered oil seed plants, such as cotton, soybean, safflower, sunflower, Brassica, maize, alfalfa, palm, coconut, etc. In certain embodiments, commercial products are derived from a genetically engineered oil-seed rape, sugar beet, maize, sunflower, soybean, or sorghum. In some embodiments, commercial products are derived from a genetically engineered leguminous plants, such as beans and peas (e.g., guar, locust bean, fenugreek, soybean, garden beans, cowpea, mungbean, lima bean, fava bean, lentils, chickpea, etc.)

In certain embodiments, commercial products are derived from a genetically engineered horticultural plant of the present invention, such as lettuce, endive, and vegetable brassicas including cabbage, broccoli, and cauliflower, and carnations and geraniums; tomato, tobacco, cucurbits, carrot, strawberry, sunflower, tomato, pepper, chrysanthemum, poplar, eucalyptus, and pine.

In still other embodiments, commercial products are derived from a genetically engineered corn (Zea mays), canola (Brassica napus, Brassica rapa ssp.), alfalfa (Medicago sativa), rice (Oryza sativa), rye (Secale cereale), sorghum (Sorghum bicolor, Sorghum vulgare), sunflower (Helianthus annuus), wheat (Triticum aestivum), soybean (Glycine max), tobacco (Nicotiana tabacum, Nicotiana benthamiana), potato (Solanum tuberosum), peanuts (Arachis hypogaea), cotton (Gossypium hirsutum), sweet potato (Ipomoea batatus), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Peryea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Prunus amygdalus), sugar beets (Beta vulgaris), oats, barley, Arabidopsis spp., vegetables, ornamentals, and conifers.

5.3. Components of the Target System

The TARGET system utilizes a nucleic acid encoding a chimeric protein comprising a transcription factor fused to a domain comprising an inducible cellular localization signal and an independently expressed selectable marker. Nucleic acids for use with the target system may be plasmids or other appropriate nucleic acid constructs as described in Section 5.2.3. The TARGET system also comprises methods of measuring mRNA expression levels and may additionally comprise methods of detecting TF binding to gene targets.

5.3.1. Transcription Factors

The transcription factor component chimeric protein encoded by the nucleic acid constuct may be, but is not limitied to, one of those listed in Table 3. The transcription factor used is not limited to nuclear transcription factors, but may also include proteins that modulate mitochondrial or chloroplast gene expression.

5.3.2. Localization Signals and Inducing Agents

The glucorticoid receptor (GR) may be used as the inducible cellular localization signal in the chimeric protein encoded by the nucleic acid construct. In the case of the a TF-GR chimeric protein, dexamethasone may be used as the inducing agent. Alternately, another glucocorticoid may be used instead of dexamethasone. Treatement with dexamethasone releases the glucocorticoid receptor from sequestration in the cytoplasm, allowing the TF-GR fusion protein to access its target genes (e.g., in the nucleus). The GR is not the only such inducible cellular localization signal that may be used in this method. Any receptor component or other protein known in the art that is capable of being released from sequestration or otherwise re-localized to the destination of the transcription factor component by treatment of the protoplasts with an inducing agent may potentially be used in the TARGET system.

5.3.3. Expression System and Selectable Markers

Using any gene transfer technique, such as the above-listed techniques (of Section 5.2), an expression vector harboring the nucleic acid may be transformed into a cell to achieve temporary or prolonged expression. Any suitable expression system may be used, so long as it is capable of undergoing transformation and expressing of the precursor nucleic acid in the cell. In one embodiment, a pET vector (Novagen, Madison, Wis.), or a pBI vector (Clontech, Palo Alto, Calif.) is used as the expression vector. In some embodiments an expression vector further encoding a green fluorescent protein (“GFP”) is used to allow simple selection of transfected cells and to monitor expression levels. Non-limiting examples of such vectors include Clontech's “Living Colors Vectors” pEYFP and pEYFP-C.

The recombinant construct of the present invention may include a selectable marker for propagation of the construct. For example, a construct to be propagated in bacteria preferably contains an antibiotic resistance gene, such as one that confers resistance to kanamycin, tetracycline, streptomycin, or chloramphenicol. Suitable vectors for propagating the construct include plasmids, cosmids, bacteriophages or viruses, to name but a few.

In some embodiments, the selectable marker encoded by the nucleic acid molecule used in the method of the invention is a fluorescent selection marker. A fluorescent selection marker that can be used in the method of the invention includes, but is not limited to, green fluorescent protein, yellow fluorescent protein, red fluorescent protein, cyan fluorescent protein, or blue fluorescent protein. In a specific embodiment, the fluorescent selection marker used in the method of the invention is red fluorescent protein. In certain embodiments, the step of detecting host cells that express the selectable marker is performed by Fluorescence Activated Cell Sorting (FACS). Any selectable marker known in the art that may be encoded in the nucleic acid construct and which is selectable using a cell sorting or other selection technique may be used to identify those cells that have expressed the nucleic acid construct containing the chimeric protein.

In addition, the recombinant constructs may include plant-expressible selectable or screenable marker genes for isolating, identifying or tracking of plant cells transformed by these constructs. Selectable markers include, but are not limited to, genes that confer antibiotic resistances (e.g., resistance to kanamycin or hygromycin) or herbicide resistance (e.g., resistance to sulfonylurea, phosphinothricin, or glyphosate). Screenable markers include, but are not limited to, the genes encoding .beta.-glucuronidase (Jefferson, 1987, Plant Molec Biol. Rep 5:387-405), luciferase (Ow et al., 1986, Science 234:856-859), B and C1 gene products that regulate anthocyanin pigment production (Goff et al., 1990, EMBO J 9:2517-2522).

In some cases, a selectable marker may be included with the nucleic acid being delivered to the cell. A selectable marker may refer to the use of a gene that encodes an enzymatic or other detectable activity (e.g., luminescence or fluorescence) that confers the ability to distinguish cells expressing the nucleic acid construct from those that do not. A selectable marker may confer resistance to an antibiotic or drug upon the cell in which the selectable marker is expressed. Selectable markers may be “dominant” in some cases; a dominant selectable marker encodes an enzymatic or other activity (e.g., luminescence or fluorescence) that can be detected in any cell or cell line.

In some embodiments, the marker gene is an antibiotic resistance gene whereby the appropriate antibiotic can be used to select for transformed cells from among cells that are not transformed. Examples of suitable selectable markers include adenosine deaminase, dihydrofolate reductase, hygromycin-B-phosphotransferase, thymidine kinase, xanthine-guanine phospho-ribosyltransferase and amino-glycoside 3′-O-phosphotransferase II. Other suitable markers will be known to those of skill in the art.

5.3.4. Detecting the Level of mRNA Expressed in Host Cells

The methods of the present invention comprise a step of detecting the level of mRNA expressed in the host cells of the invention.

In some embodiments, the level of mRNA expressed in host cells is determined by quantitative real-time PCR (qPCR), a method for DNA amplification in which fluorescent dyes are used to detect the amount of PCR product after each PCR cycle. (Higuchi et al., 1992; Simultaneous amplification and detection of specific DNA-sequences. Bio-Technology 10(4), 413-417].). The qPCR method has become the tool of choice for many scientists because of method's dynamic range, accuracy, high sensitivity, specificity and speed. Quantitative PCR is carried out in a thermal cycler with the capacity to illuminate each sample with a beam of light of a specified wavelength and detect the fluorescence emitted by the excited fluorochrome. The thermal cycler is also able to rapidly heat and chill samples thereby taking advantage of the physicochemical properties of the nucleic acids and DNA polymerase.

In some embodiments, the level of mRNA expressed in host cells is determined by high high throughput sequencing (Next-generation sequencing; also ‘Next-gen sequencing’ or NGS).=NGS methods are highly parallelized processes that enable the sequencing of thousands to millions of molecules at once. Popular NGS methods include pyrosequencing developed by 454 Life Sciences (now Roche), which makes use of luciferase to read out signals as individual nucleotides are added to DNA templates, Illumina sequencing that uses reversible dye-terminator techniques that adds a single nucleotide to the DNA template in each cycle and SOLiD sequencing by Life Technologies that sequences by preferential ligation of fixed-length oligonucleotides.

In some embodiments, the level of mRNA expressed in host cells is determined by gene microarrays. A microarray works by exploiting the ability of a given mRNA molecule to bind specifically to, or hybridize to, the DNA template from which it originated. By using an array containing many DNA samples, it can be determined in a single experiment, the expression levels of hundreds or thousands of genes within a cell by measuring the amount of mRNA bound to each site on the array. With the aid of a computer, the amount of mRNA bound to the spots on the microarray is precisely measured, generating a profile of gene expression in the cell.

5.3.5. Detecting TF Binding to Gene Targets

In some embodiments, the method comprises detection of the level of TF binding to gene targets by ChIP-Seq analysis. ChIP-Seq analysis utilizes chromatin immunoprecipitation in parallel with DNA sequencing to map the binding sites of a TF or other protein of interest. First, protein interactions with chromatin are cross-linked and fragmented. Then, immunoprecipitation is used to isolate the TF with bound chromatin/DNA. The associated chromatin/DNA fragments are sequenced to determine the gene location of protein binding. Other assays known in the art may be used to detect the location of TF binding to genomic regions of DNA.

In some embodiments, the yeast one hybrid method may be used. The yeast one hybrid method detects protein-DNA interactions, and may be adapted for use in plants. The DNA binding domains unveiled by ChIP-Seq may be cloned upstream of a reporter gene in a vector or may be introduced into the plant genome by homologous recombination, which allows the transcription factor to interact with the DNA element in a natural environment. A fusion protein containing a constitutive TF activation domain and the DNA binding domain of the TF of interest may then be expressed, and the interaction of the binding domain with the DNA will be detected by reporter gene expression. The yeast one hybrid method can thus be used in some embodiments as a way to interrogate the relationship between binding and activation, as only the binding domain of the TF of interest is used in the fusion protein in the heterologous system.

5.3.6. Identifying Conserved Connections Across Species

In some embodiments, gene networks conserved between Arabidopsis (or another model species) and a species of interest may be determined by a data mining approach. In this approach, Arabidopsis plants are grown under the same conditions as plants from another species of interest, including perturbation of environmental signals (e.g. nitrogen). RNA is then extracted from the roots and shoots of the plants, and cDNA synthesized from the extracted RNA. A microarray analysis and filtering approach may be used to determine the genes of each species regulated by the environmental signal when compared with control conditions. An ortholog analysis may then determine the genes orthologous between the two species. Data integration and network analysis then allows for the determination of a core translational network. In some embodiments, the response genes in a species of plant for which a protoplast system is not feasible may be discovered by using such a data mining approach, as described, in combination with the TARGET system for Arabidopsis or another species used as a model.

### 6. EXAMPLE 1

### 6.1. Introduction

A rapid technique to study the genome-wide effects of TF activation in protoplasts that uses transient expression of a glucocorticoid receptor (GR)-tagged TF has been developed in the present invention. This system can be used to rapidly retrieve information on direct target genes in less than two week's time. As a proof-of-principle candidate, the well-studied transcription factor, Abscicic acid insensitive 3 (ABI3; Koommeef et al., 1989, Plant physiology, 90:463-469; Mönke et al., 2012, Nucleic acids research 40:8240-8254) was used. The de novo identification of the abscisic acid response element (ABRE) and a majority of the previously classified direct targets was established by use of this method. This technique was named TARGET, for Transient Assay Reporting Genome-wide Effects of Transcription factors.

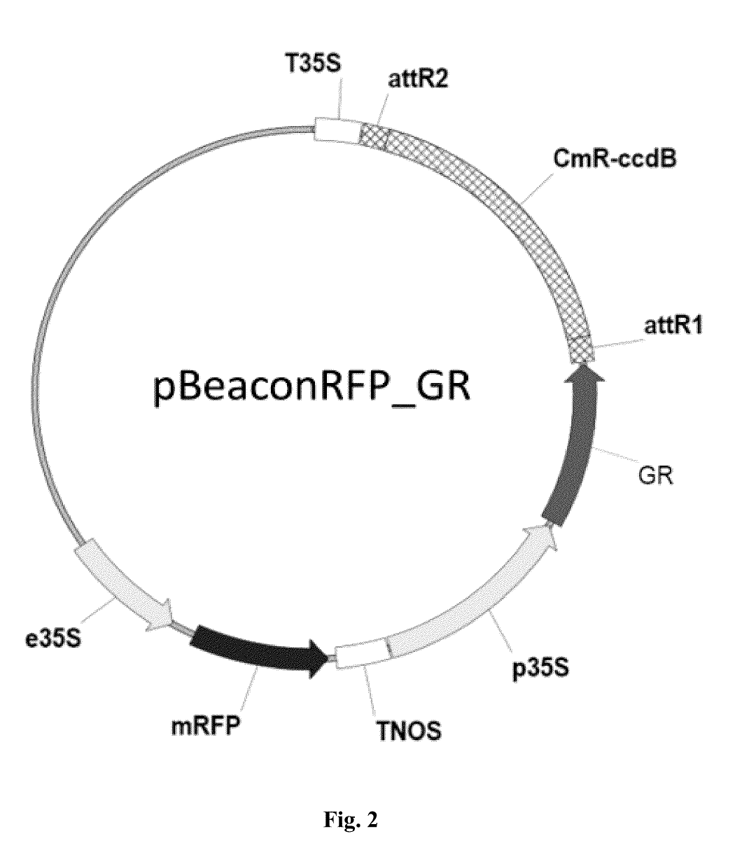

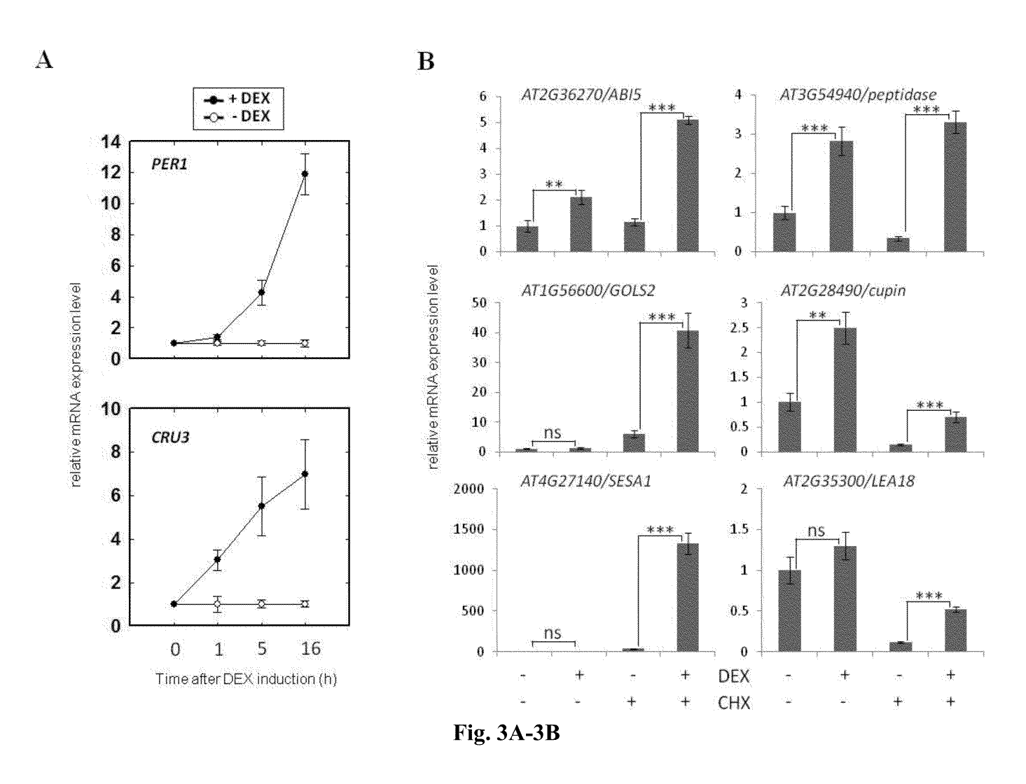

Technically, plant protoplasts are transfected with a plasmid (pBeaconRFP_GR) that expresses the TF-of-interest fused to GR, which allows the controlled entry of the chimeric GR-TF into the nucleus by addition of the GR-ligand dexamethasone (DEX; Schena and Yamamoto, 1988, Science 241:965-967). In addition, the vector contains a separate expression cassette with a positive fluorescent selection marker (red fluorescent protein; RFP) which enables fluorescence activated cell sorting (FACS) of successfully transformed protoplasts (see FIG. 2; Bargmann and Birnbaum, 2009, Plant physiology 149:1231-1239). This purification step allows reliable qPCR or transcriptomic analysis of multiple independent transfections, which would otherwise be hampered by the presence of a population of untransformed cells that varies from experiment to experiment. Lastly, the effect of target gene induction by DEX treatment is measured in the presence or absence of the translation inhibitor cycloheximide (CHX), allowing for the distinction of direct and indirect target genes of the TF under study. pBeaconRFP_GR-ABI3 was used to transfect protoplasts prepared from the roots of Arabidopsis seedlings, where ABI3, known largely for its role in seed development, has also been shown to be involved in development (Brady et al., 2003, The Plant journal: for cell and molecular biology 34:67-75).

### 6.2. Materials and Methods

Plant Materials and Treatment.

Wild-type Arabidopsis thaliana seed (Col-0, Arabidopsis Biological Resource Center) was sterilized by 5 min incubation with 96% ethanol followed by 20 min incubation with 50% household bleach and rinsing with sterile water. Seeds were plated on square 10×10 cm plates (Fisher Scientific) with MS-agar (2.2 g/l Murashige and Skoog Salts [Sigma-Aldrich], 1% [w/v] sucrose, 1% [w/v] agar, 0.5 g/l MES hydrate [Sigma-Aldrich], pH 5.7 with KOH) on top of a sterile nylon mesh (NITEX 03-100/47, Sefar filtration Inc.) to facilitate harvesting of the roots. Seeds were plated in two dense rows. Plates were vernalized for 2 days at 4° C. in the dark and placed vertically in an Advanced Intellus environmental controller (Percival) set to 35 μmol/m₂*sec₋₁ and 22° C. with an 18 h-light/6 h-dark regime.

Vector Construction.

pBeaconRFP_GR was constructed by PCR amplification of the glucocorticoid receptor from pJCGLOX (Joubes et al., 2004, The Plant Journal 37: 889-896) with primers GR-F and GR-R, both with an SpeI restriction site, using Phusion polymerase (New England Biolabs). The PCR product was ligated into the SpeI site upstream of the GATEWAY (Invitrogen) cassette in pBeaconRFP (Bargmann and Birnbaum, 2009; Plant physiology 149:1231-1239). The orientation of the insert was checked by PCR. The pBeaconRFP_GR vector (as well as the pMON999_mRFP control vector, containing only 35S::mRFP) will be made available through the VIB website: http://gateway.psb.ugent.be/.

ABI3 cDNA was PCR amplified with primers ABI3_AttB1 and ABI3_AttB2, and subsequently re-amplified with primers AttB1 and AttB2 using Phusion polymerase. The PCR product was recombined into pDONR221 using BP clonase and subsequently shuttled into pBeaconRFP_GR with LR clonase (Invitrogen).

Protoplast Preparation, Transfection, Treatment and Cell Sorting.

Protoplast were prepared, transfected and sorted as described in Bargmann and Birnbaum, 2009; Plant physiology 149:1231-1239; and Bargmann and Birnbaum, 2010, JoVE. Briefly, roots of 10-day-old seedling were harvested and treated with cell wall digesting enzymes (Cellulase and Macerozyme; Yakult, Japan) for 3 hours. Cells were filtered, washed and 106 cells were transfected with a polyethylene glycol treatment using 50 μg of plasmid DNA and incubated at room temperature overnight. Protoplast suspensions were pretreated with 35 μM cycloheximide (CHX; Sigma-Aldrich) for 30 min, after which 10 μM dexamethasone (DEX; Sigma-Aldrich) was added and cells were incubated at room temperature. Controls were treated with solvent alone. A 10 mM DEX stock was dissolved in ethanol and a 50 mM CHX stock was dissolved in dimethylsulfoxide, both were stored at −20° C. All transfections and treatments were performed in triplicate. Treated protoplasts suspensions were sorted with a FACSAria (BD Biosciences), using 488 nm excitation and measuring emission at 530/30 nm for green fluorescence and 610/20 nm for red fluorescence. RFP-positive cells were sorted directly into RNA extraction buffer. Twenty thousand RFPpositive cells (+/−10% of sorted events were RFP-positive under these experimental conditions) were then isolated by FACS and RNA was extracted for transcript analysis by qPCR.

A temporal qPCR analysis of PER1 and CRU3 induction by DEX in the presence of CHX was performed after a 1-hour, 5-hour and overnight (16-hour) incubation (see FIG. 3A). Results indicated that, although induction could be seen as early as 1 hour after the addition of DEX for CRU3, the expression of both PER1 and CRU3 continued to increase after 5 and 16 hours (see FIG. 3A). In order to achieve a large fold-change in expression between control and treatment, microarray analysis was performed after an overnight treatment.

qPCR and Microarray Analysis.

RNA was extracted using an RNeasy Micro Kit with RNase-free DNase Set according to the manufacturer's instructions (QIAGEN). RNA was quantified with a Bioanalyzer (Agilent Technologies). Gene expression was determined by quantitative real-time PCR (LightCycler; Roche Diagnostics) using gene-specific primers and LightCycler FastStart DNA Master SYBR Green (Roche Diagnostics). Expression levels of tested genes were normalized to expression levels of the ACT2/8 and CLATHRIN genes as described in (Krouk et al., 2006 Plant Physiol 142:1075-1086). For microarray analysis, RNA was amplified and labeled with WT-Ovation Pico RNA Amplification System and FL-Ovation cDNA Biotin Module V2, respectively (NuGEN). The labeled cDNA was hybridized, washed and stained on an ATH-121501 Arabidopsis full genome microarray using a Hybridization Control Kit, a GeneChip Hybridization, Wash, and Stain Kit, a GeneChip Fluidics Station 450 and a GeneChip Scanner (Affymetrix). The microarray data reported in this paper have been deposited in the Gene Expression Omnibus (GEO, http://www.ncbi.nlm.nih.gov/geo/) database, (accession #GSE33344). Raw microarray data was normalized using MAS5.0 (scaling factor of 250, Flexarray; http://www.gqinnovationcenter.com/services/bioinformatics/flexarray/index.aspx?l=e). Data was logged prior to running a Tukey post hoc test on the significance coefficients of a two way ANOVA carried out on CHX versus DEX treatment (in-house [R] script) for differential responses to DEX with or without CHX on non-ambiguous probesets. Heatmaps were created using Multiple Experiment Viewer software (TIGR; http://www.tm4.org/mev/). For the overlap analysis with previously identified targets of ABI3 (Mönke et al., 2012, Nucleic acids research 40:8240-8254), VP1 (Suzuki et al., 2003, Plant physiology 132:1664-1677) and ABI5 (Reeves et al., 2011, Plant molecular biology, 75:347-363), distance between non-parametric distributions (one from the overlap of sampled input gene sets and one from two randomly sampled sets of genes represented on the ATH1 array) was calculated using the genesect [R] script (Krouk et al., 2010, Genome biology 11:R123). For the overlap with VP1 targets, the background consisted of genes represented on both the ATH1- and the 8 k AG array [Affymetrix] used by Suzuki and co-workers.

GO-Term and Promoter Analysis.

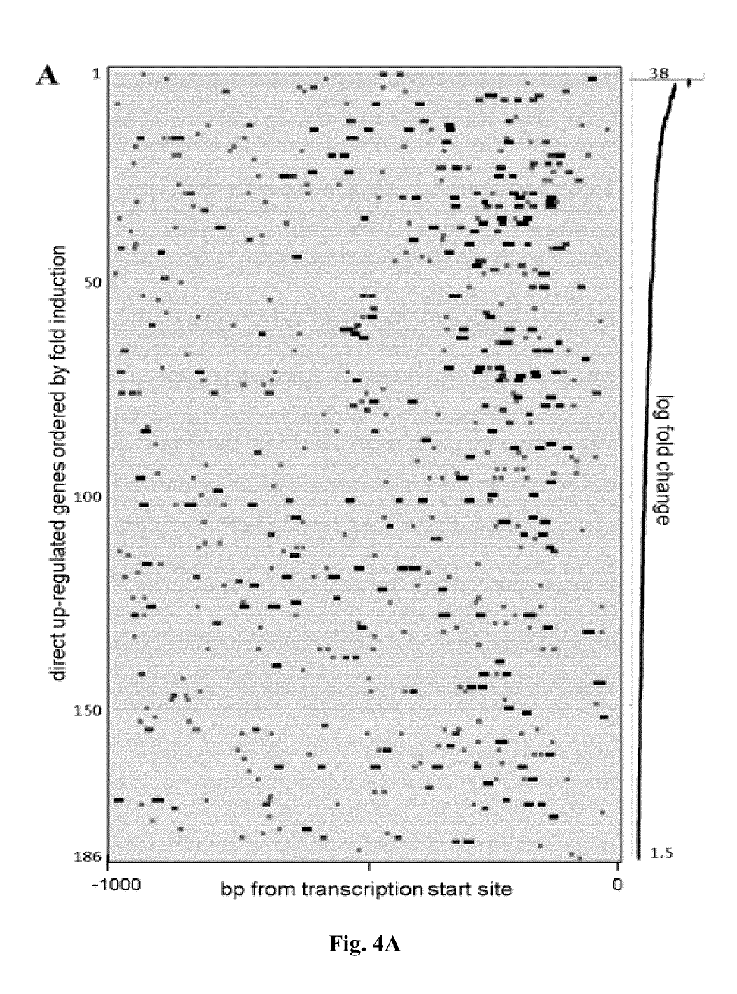

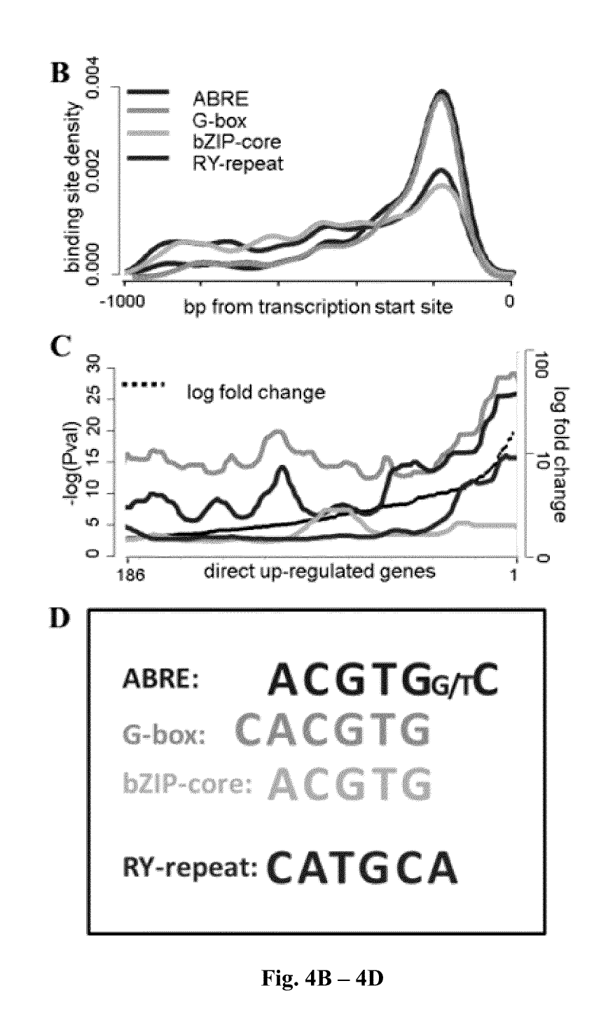

GO-term analysis was performed online using the BioMaps function on the VirtualPlant website (www.virtualplant.org) with a default corrected p-value cutoff on the Fisher exact test of p<10-3 (Katari et al., 2010; Plant Physiology, 152:500-515). To determine enrichment of known promoter motifs, the number of 1 kb upstream promoters, out of the top fifty ABI3 up-regulated genes, having one or more of the motifs described in the PLACE database was counted (http://www.dna.affrc.go.jp/PLACE/). p-values were generated using hypergeometric distribution, and values were FDR corrected using an FDR q-value cutoff of 0.01.promoter element enrichment analysis was performed using [R] (http://www.r-project.org/). For the sliding window analysis for promoter element enrichment (see FIG. 4), significance was calculated using the hypergeometric test, comparing the number of motif occurrences in a 30-gene window to the number expected by chance, which was derived from the propensity of the motif in the promoters of all genes nonambiguously represented on the ATH1 chips. The search for recurring promoter motifs was performed using the Cistome website (http://bar.utoronto.ca/cistome/cgibin/BAR_Cistome.cgi). Motif Sampler and MEME were used to look for recurring 8-mer motifs in the 1000 bp upstream of the top fifty direct up-regulated genes with the following significance parameters: Ze cutoff 3.0, functional depth cutoff 0.35, proportion of genes the motif should be found in 0.5.

### 6.3. Results

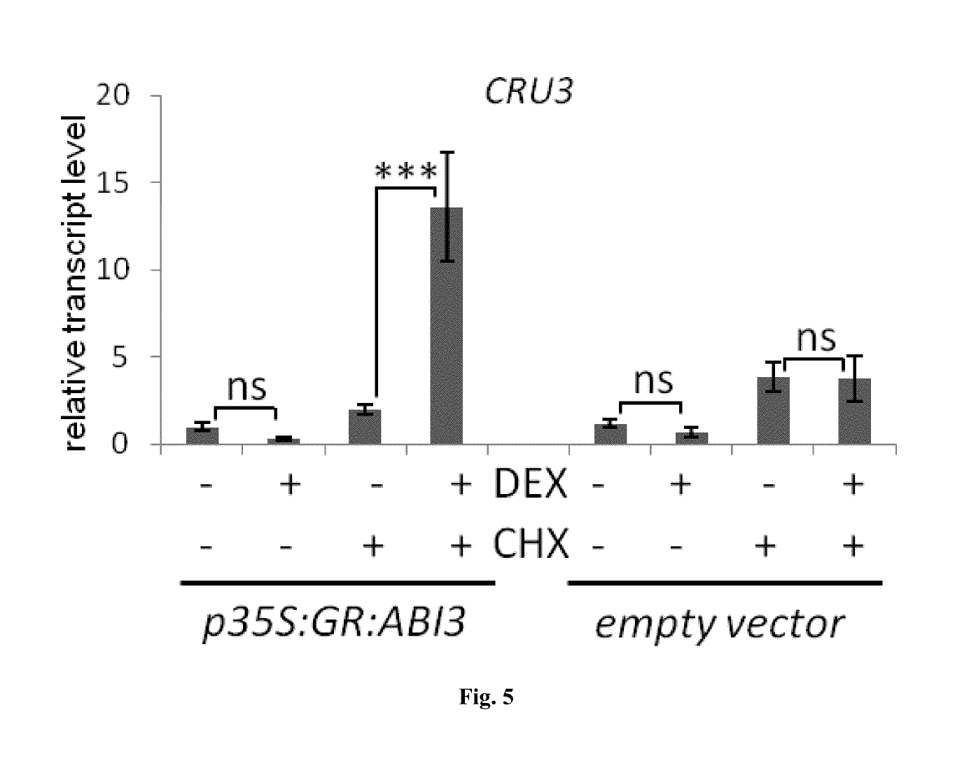

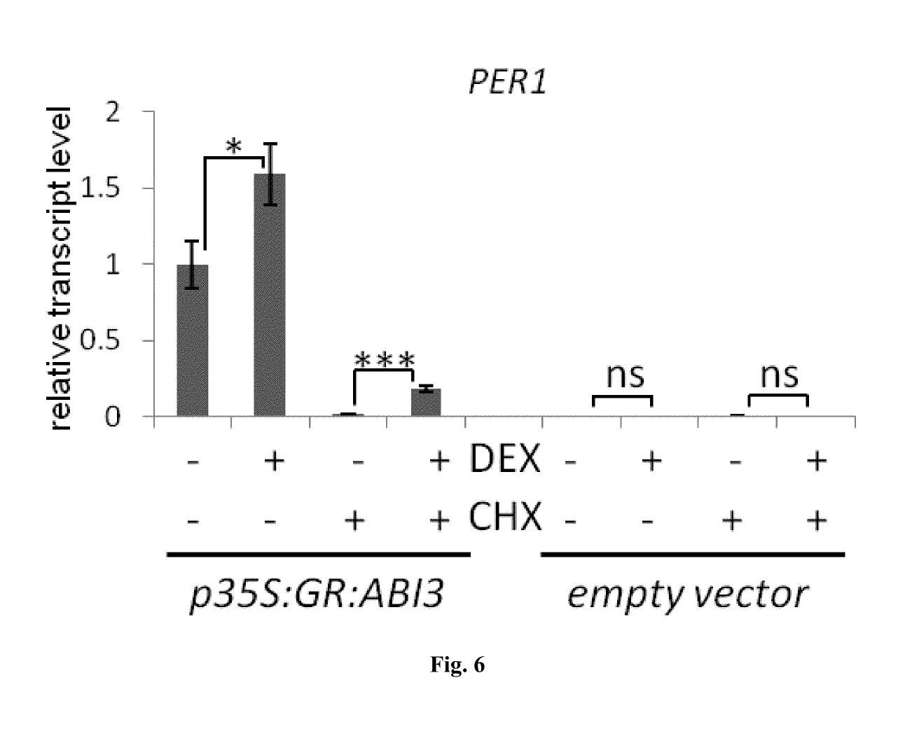

As a first test of the TARGET system, the expression of known direct ABI3 targets PER1 and CRU3 were assayed by qPCR. Compared to control gene expression, both PER1 and CRU3 showed significant induction of transcript levels upon DEX treatment in the ABI3-GR transfected protoplasts in the presence of CHX (FIGS. 5 and 6). PER1 and CRU3 expression in protoplasts transformed with an empty vector control showed no significant induction by DEX treatment (FIGS. 5 and 6). Significant induction of CRU3 expression could only be measured when CHX was present, indicating that the effects of CHX may in some cases facilitate ABI3 function. Enhancement of ABA signaling output by protein synthesis inhibitors, that could explain this phenomenon, has been noted before by independent studies (Reeves et al., 2011, Plant molecular biology 75:347-363). For the transcriptomic analysis, using ATH1 Genome Array chips, a two-way analysis of variance (ANOVA) was performed, followed by a Tukey post hoc test to identify genes whose expression is differentially regulated in response to DEX treatment in the absence or presence of CHX (p<0.05, fold change>1.5). Genes found to be significantly regulated by DEX treatment in the empty vector control were omitted from further analysis. This analysis yielded a total of 668 unique genes whose expression was affected by DEX-induced nuclear localization of ABI3; 227 regulated genes without CHX and 458 regulated genes with CHX (microarray results were validated by qPCR). There was just a 17-gene overlap with and without CHX, reiterating that (as was seen for CRU3 in preliminary qPCR analysis) there are many genes whose response to GR-ABI3 was facilitated by the presence of the protein synthesis inhibitor CHX. The 210 genes regulated only in the absence of CHX were categorized as putative indirect targets of ABI3, whereas the 458 genes regulated in the presence of CHX (186 induced and 272 repressed genes) were designated as putative direct targets of ABI3.

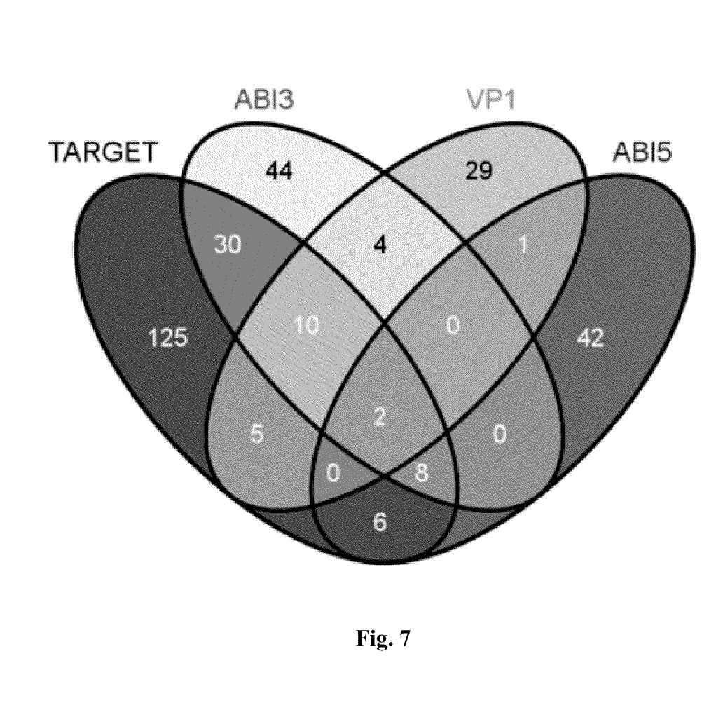

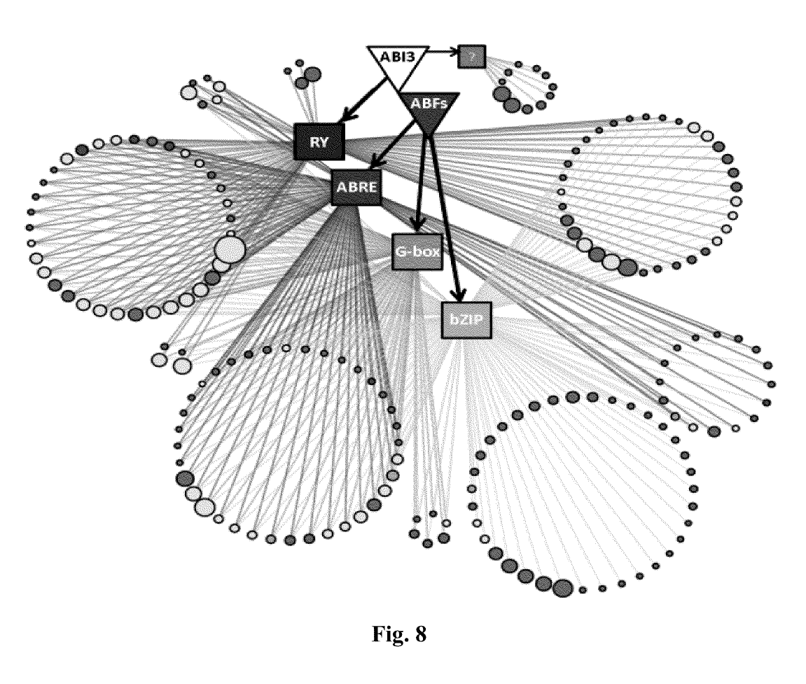

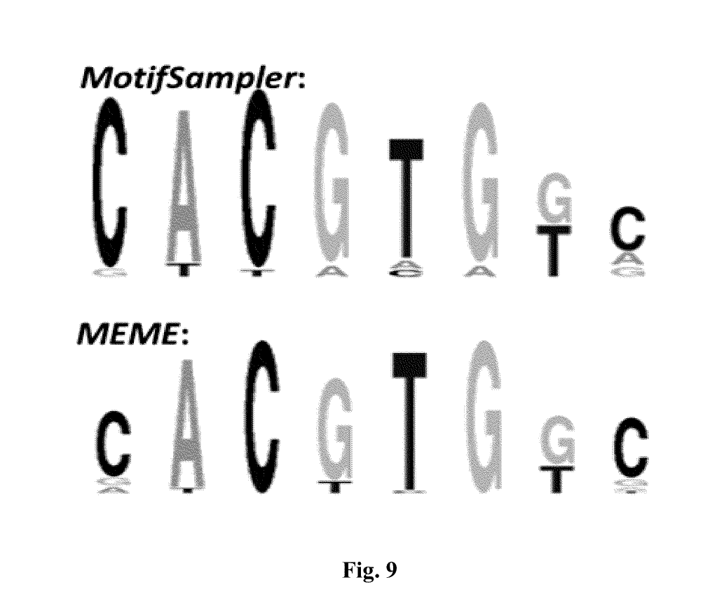

The list of 186 putative direct up-regulated genes was highly significantly enriched for genes previously identified as direct targets of ABI3 in whole plant studies (Ze=54.3), as well as targets of the maize homolog VIVIPAROUS1 (Ze=20.8) and co-regulator ABI5 (Ze=20.9) (FIGS. 7 and 8; (Mönke et al., 2012, Nucleic acids research 40:8240-8254; Reeves et al., 2011, Plant molecular biology 75:347-363; Suzuki et al., 2003, Plant physiology 132:1664-1677). These substantial intersections indicate that the activation of ABI3 in protoplasts reflects the effects attributed to this transcriptional regulator in in planta studies. The list also showed a significant overrepresentation of GO-terms, including response to ABA, response to water deprivation, lipid storage and embryo development (no significant overlap or enrichments were found in the lists of indirect targets or direct down-regulated targets). Furthermore, promoter analysis of the fifty most strongly induced direct up-regulated genes found significant enrichment of previously identified ABRE-like elements and the RY-repeat motif (FIG. 8). De novo searches for recurring motifs within these promoters (using two independent algorithms, MEME and MotifSampler) yielded the recovery of the CACGTGKC ABRE (FIG. 9). These results show the TARGET system can be used successfully to investigate TF function in protoplasts with significance to whole plants.

### 6.4. Discussion

One advantage of the TARGET system lies in the speed at which identification of genome-wide TF targets can be performed. A candidate TF can now be scrutinized for its target genes in a genome in a matter of weeks rather than the months required for the generation of stable transgenic plant lines. The TARGET transient transformation system can also be used purely as a verification of specific TF-target interactions by qPCR, much as yeast-one-hybrid (Y1H) assays are often used, but now in the context of endogenous gene activation in plant cells rather than promoter binding in a yeast strain. The TARGET approach brings the convenience of microbiological systems like Y1H to the genome-wide transcriptomic capabilities of in planta studies. Another advantage of the use of protoplast transformation in the TARGET system is that it can be done in a wide range of species where the generation of transgenic plant lines is either impossible or problematic and more time-consuming (Sheen et al., 2001, Plant physiology 127:1466-1475). The TARGET system combined with RNA sequencing, can enable rapid and systematic assessment of TF function in numerous plant species, for example in important crop model species.

This system is not a replacement for in-depth studies using transcriptional- and chromatin immuno-precipitation (ChIP) analyses in transgenic plants. Rather, TARGET is rapid tool for GRN investigations that may have uses in particular circumstances. There are considerations associated with the use of this system. On its own, a genome-wide analysis will yield results that contain false-positives and false-negatives. Identification of direct regulated genes by TARGET is therefore not unequivocal, additional assays for direct TF-target interaction (e.g. ChIP, Y1H, gel shift assays) are required for definitive identification of TF targets. The functionality of the chimeric GR-TF is not tested in this system, other than by the substance of the results. CHX treatment by itself may have effects on transcription that influence the DEX effect on certain direct target genes. Lastly, the cellular dissociation procedure itself may induce gene expression responses that could conceal the effects of TF activation. One can envisage two ways of using the TARGET system; either in combination with other techniques to get high confidence target lists for a particular TF, or as a high-throughput analysis of numerous TFs in a given GRN to get a broad view of putative interactions.

Overall, the results presented here demonstrate that TARGET represents a novel and rapid transient system for TF investigation that can be used to help map GRN. Important indications of TF operation, such as direct target genes, biological function by GO-term associations and cis-regulatory elements involved in its action, can be obtained in a rapid and straightforward manner. The proof-of-principle analysis with ABI3 offers a new dataset of transcripts affected by this TF, adding to the understanding of the downstream significance of this central regulator.

The pBeaconRFP_GR vector will be made available through the VIB website (http://gateway.psb.ugent.be/).

### 7. EXAMPLE 2

### 7.1. Introduction

Evidence for temporal, signal induced TF-target associations that involve the rapid and transient induction of genes related to the signal has been developed in the present invention. This discovery was enabled by a combination of conceptual and technical advances in a cell-based system, which enabled overexpression of a specific TF of interest and temporal induction of its nuclear localization. By temporally inducing TF nuclear localization using dexamethasone (DEX) in the presence of cycloheximide (CHX) to block translation, identification of the primary targets of a TF of interest was possible, based on either TF-regulation or TF-binding assayed in the same samples, exposed to a signal. Moreover, the perturbation of both the TF and the signal it transduces uncovered three distinct TF modes-of-action, “poised”, “active” and “transient”, the latter encompassing signal-dependent, transient TF-target associations. This discovery was made for bZIP1 (BASIC LEUCINE ZIPPER 1), a TF implicated as an integrator of cellular and metabolic signaling in Arabidopsis and shared in other eukayrotes (Weltmeier et al., 2008, Plant Molecular Biology 69:107; Sun et al., 2011, Journal of Plant Research 125:429; Baena-Gonzalez et al., 2007, Nature 448:938; Kietrich et al., 2011, The Plant Cell 23:381; Kang et al., 2010, Molecular Plant 3:361; Gutierrez et al., 2008, Proc. Natl. Acad. Sci. U.S.A., 105:4939; Obertello et al., 2010, BMC systems biology 4:111). The discovery of this new class of“transient”, signal-induced TF-target interactions opens a window into TF network dynamics that has been missed in previous TF studies in plants and animals. The inclusion of such context-dependent TF-target interactions in GRNs, will improve the predictive capability of GRN models to generate hypotheses that will direct future experimental efforts in living systems.

### 7.2. Materials and Methods

Plant Materials and DNA Constructs.

Wild-type Arabidopsis thaliana seeds [Columbia ecotype (Col-0)] were vapor-phase sterilized, vernalized for 3 days, then 1 ml of seeds were sown on 24 agar plates containing MS [2.2 g/l custom made Murashige and Skoog salts without N or sucrose [Sigma-Aldrich]; 1% [w/v] sucrose; 0.5 g/l MES hydrate [Sigma-Aldrich]; 1 mM KNO₃; 2% [w/v] agar; pH 5.7 with HCl]. Plants were grown vertically in an Intellus environment controller [Percival Scientific, Perry, Iowa] set to 35 μmol m⁻² s⁻¹ and 16 h-light/8 h-dark regime at constant 22° C. bZIP1 [At5g49450] cDNA in pENTR was obtained from the REGIA collection (Paz-Ares et al., 2002, Comparative and functional genomics 3:102) and was then cloned into the destination vector pBeaconRFP_GR (Bargmann et al., 2013, Molecular Plant 6(3):978) by LR recombination [Life Technologies].

Protoplast Preparation, Transfection, Treatment and Cell Sorting.

Protoplasts were prepared, transfected and sorted as previously described (Bargmann et al., 2013, Molecular Plant 6(3):978; Yoo et al., 2007, Nature Protocols 2:1565; Bargmann et al., 2009, Plant physiology 149:1231). Briefly, roots of 10-day-old seedlings were harvested and treated with cell wall digesting enzymes [Cellulase and Macerozyme; Yakult, Japan] for 4 h. Cells were filtered and washed then transfected with 40 μg of pBeaconRFP_GR::bZIP1 plasmid DNA per 1×10⁶ cells facilitated by polyethylene glycol treatment [PEG; Fluka 81242] for 25 minutes (Bargmann et al., 2013, Molecular Plant 6(3):978). Cells were washed drop-wise, concentrated by centrifugation, then resuspended in wash solution for overnight incubation at room temperature. Protoplast suspensions were treated sequentially with a N-signal treatment of either a 20 mM KNO₃ and 20 mM NH₄NO₃ solution [N] or 20 mM KCl [control] for 2 h, either cycloheximide [CHX][35 μM in DMSO; Sigma-Aldrich] or solvent alone as mock for 20 min, and then with either dexamethasone [DEX][10 μM in EtOH; Sigma-Aldrich] or solvent alone as mock for 4 h at room temperature. Treated protoplast suspensions were sorted as in (Bargmann et al., 2009, Plant physiology 149:1231): approximately 10,000 RFP-positive cells were sorted directly into RLT buffer [QIAGEN].

RNA Extraction and Microarray.

RNA was extracted from protoplasts [6 replicates: 3 treatment replicates and 2 biological replicates] using an RNeasy Micro Kit with RNase-free DNaseI Set [QIAGEN] and quantified on a Bioanalyzer RNA Pico Chip [Agilent Technologies]. RNA was then converted into cDNA, amplified and labeled with Ovation Pico WTA System V2 [NuGEN] and Encore Biotin Module [NuGEN], respectively. The labeled cDNA was hybridized, washed and stained on an ATH1-121501 Arabidopsis Genome Array [Affymetrix] using a Hybridization Control Kit [Affymetrix], a GeneChip Hybridization, Wash, and Stain Kit [Affymetrix], a GeneChip Fluidics Station 450 and a GeneChip Scanner [Affymetrix].

Analysis of Microarray Data with CHX Treatment:

Microarray intensities were normalized using the GCRMA [http://www.bioconductor.org/packages/2.11/bioc/html/germa.html] package. Differentially expressed genes were then determined by a 3-way ANOVA with N, DEX and biological replicates as factors. The raw p-value from ANOVA was adjusted by False Discovery Rate [FDR] to control for multiple testing (Benjamini et al., 2005, Genetics 171:783). Genes significantly regulated by N and/or bZIP1 were then selected with a FDR cutoff of 5% while genes significantly regulated by the interaction of N and bZIP1 [NXbZIP1] were selected with a p-val [ANOVA] cutoff of 0.01. Only unambiguous probes were included. Heatmaps were created using Multiple Experiment Viewer software [TIGR; http://www.tm4.org/mev/]. The significance of overlaps of gene sets were calculated using the genesect [R] script (Krouk et al., 2010, Genome Biology 11:R123) or the hypergeometric method [R].

Analysis of Microarray Data without CHX Treatment:

Analysis was identical to with CHX except a 2-way ANOVA with N and bZIP1 as factors was used to identify differentially expressed genes.

Micro Chromatin Immunoprecipitation.

For each combination of protoplast treatments (see above), an unsorted suspension of protoplasts containing approximately 5,000-10,000 GR::bZIP1 transfected cells was incubated with gentle rotation in 1% formahaldeyde in W5 buffer for 7 minutes, then washed with W5 buffer and frozen in liquid N2. μChIP was performed according to Dahl et al, 2008 (Dahl et al., 2008, Nucleic Acids Research, 36:e15) with a few modifications. The GR::bZIP1-DNA complexes were captured using anti-GR antibody [GR [P-20]-Santa Cruz biotech] bound to Protein A beads [Life Biotechnologies]. A washing step with LiCl buffer [0.25M LiCl, 1% Na deoxycholate, 10 mM Tris-HCl (pH8), 1% NP-40] was added in between the wash with RIPA buffer and TE (Dahl et al., 2008, Nucleic Acids Research, 36:e15). After elution from the beads, the ChIP material and the INPUT DNA were cleaned and concentrated using QIAGEN MinElute Kit [QIAGEN]. The protoplast suspension used for micro ChIP was not FACS sorted to maintain a comparable incubation time between the samples that were used for microarray analyses and for micro ChIP. Additionally, FACS sorting of transformed cells was not required to identify DNA targets, as it is required for microarray studies.

ChIP-Seq Library Prep.

The ChIP DNA and Input DNA were prepared for Illumina HiSeq sequencing platform following the Illumina ChIP-Seq protocol [Illumina, San Diego, Calif.] with modifications. Barcoded adaptors and enrichment primers [BiOO Scientific, TX, USA] were used according to the manufacturer's protocol. The concentration and the quality of the libraries was determined by the Qubit Fluorometric DNA Assay [InVitrogen, NY, USA], DNA 12000 Bioanalzyer chip [Agilent, CA, USA] and KAPA Quant Library Kit for Illumina [KAPA Biosystems, MA, USA]. A total of 8 libraries were then pooled equimolarly and sequenced on two lanes of an Illumina HiSeq platform for 100 cycles in paired-end configuration [Cold Spring Harbor Lab, NY].

ChIP-Seq Analysis.

Reads obtained from the four treatments were filtered and aligned to the Arabidopsis thaliana genome [TAIR10] and clonal reads were removed. The ChIP alignment data was compared to its partner Input DNA and peaks were called using the QuEST package (Valouev et al., 2008, Nature Methods 5:829.) with a ChIP seeding enrichment ≧5, and extension and background enrichments ≧2. These regions were overlapped with the genome annotation to identify genes within 500 bp downstream of the peak. The gene lists from multiple treatments were largely overlapping sets and hence were pooled to generate a single list of 850 genes that show significant binding of bZIP1. Due to technical issues, the experimental design used for ChIP-Seq precludes the observation of significant differences between the genes bound by bZIP1 under the different treatment conditions. This is because the samples fixed for ChIP included a variable number of transfected cells that were not sorted by FACS.

Cis-Element Motif Analysis.

1 Kb regions upstream of the TSS (Transcription Start Site) for target genes were extracted based on TAIR10 annotation and submitted to the Elefinder program (Li et al., 2011, Plant physiology 156:2124.) or MEME (53) to determine over-representation of known binding sites. (Different parameters used in specific cases were notified in the paper if applicable). The E-value of significance for each motif was used to cluster the occurrence of motifs in the various subsets using the HCL algorithm in MeV (Saeed et al., 2006, Methods in Enzymology 411:134). Motifs that show a higher specificity to a particular category or a sub-group were identified with the PTM algorithm in MeV. De novo motif identification was performed on 1 Kb upstream sequence of the genes regulated by bZIP1 from microarray and ChIP-Seq data separately using the MEME suite (Bailey et al., 2009, Nucleic Acids Research 37:W202).

### 7.3. Results

Perturbation of a TF and the Signal it Transduces Uncovers Context-Dependent Primary TF Target Genes.

To discern mechanisms by which TFs controlling GRNs respond to a signal perceived in vivo, both a TF (bZIP1) and a metabolic signal that it transduces (nitrogen, N) were perturbed (Gutierrez et al., 2008, Proc. Natl. Acad. Sci. U.S.A. 105:4939; Obertello et al., 2010, BMC systems biology 4:111). The Arabidopsis TF bZIP1 was transiently overexpressed as a glucocorticoid receptor fusion (35S::GR-bZIP1) in a rapid cell-based system called TARGET (Transient Assay Reporting Genome-wide Effects of Transcription factors) (Bargmann et al., 2013, Molecular Plant 6(3):978) and genome-wide responses were monitored (FIG. 1). The GR-TF fusion enabled temporal induction of the nuclear localization of the TF using dexamethasone (DEX), as performed previously in planta (Eklund et al., 2010, Plant Cell 22:349) and in the cell-based TARGET system (Bargmann et al., 2013, Molecular Plant 6(3):978). In detail, Arabidopsis root protoplast cells overexpressing the 35S::GR-bZIP fusion protein were sequentially treated as follows: i) pre-treatment with an external metabolic signal (nitrogen, +/−N), followed by ii) CHX to block the synthesis of proteins, and iii) DEX to induce bZIP1 nuclear import of the GR-TF fusion (FIG. 1). Importantly, the addition of CHX blocks translation of mRNAs of bZIP1 primary targets, enabling identification of primary TF targets based solely on their TF-induced regulation (Bargmann et al., 2013, Molecular Plant 6(3):978; et al., 2010, Plant Cell 22:349). This sequence of treatments enabled identification of i) bZIP1 primary targets based on either TF-induced gene regulation or TF-binding and ii) the “context-dependence” of TF-target gene regulation (i.e. response to both TF and signal perturbation).

Discovery of bZIP1 Primary Targets by Either Gene Regulation or Promoter Binding.

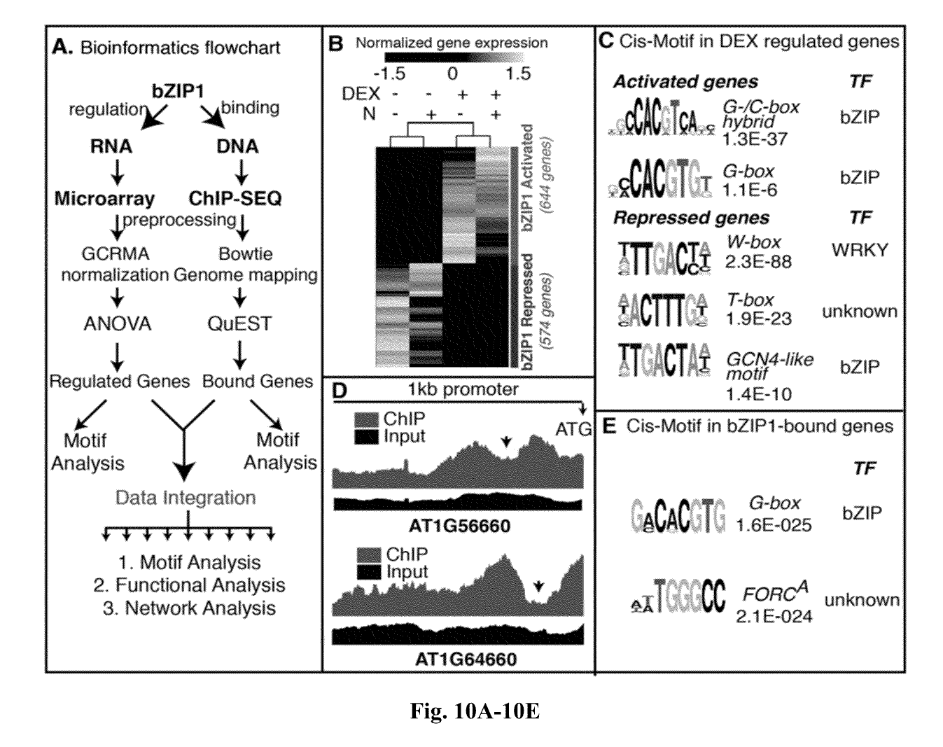

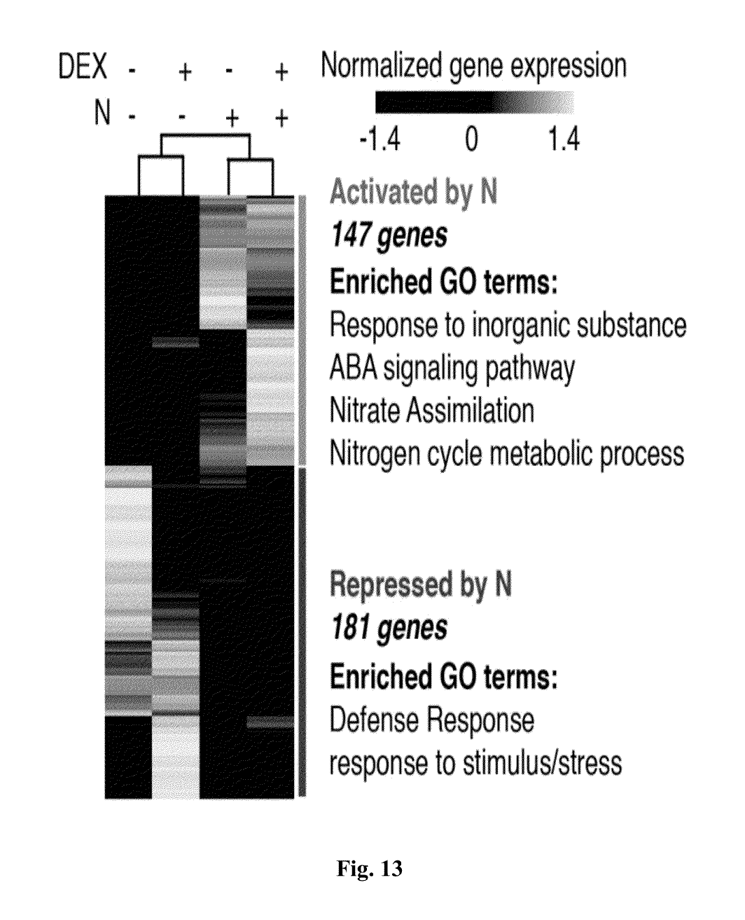

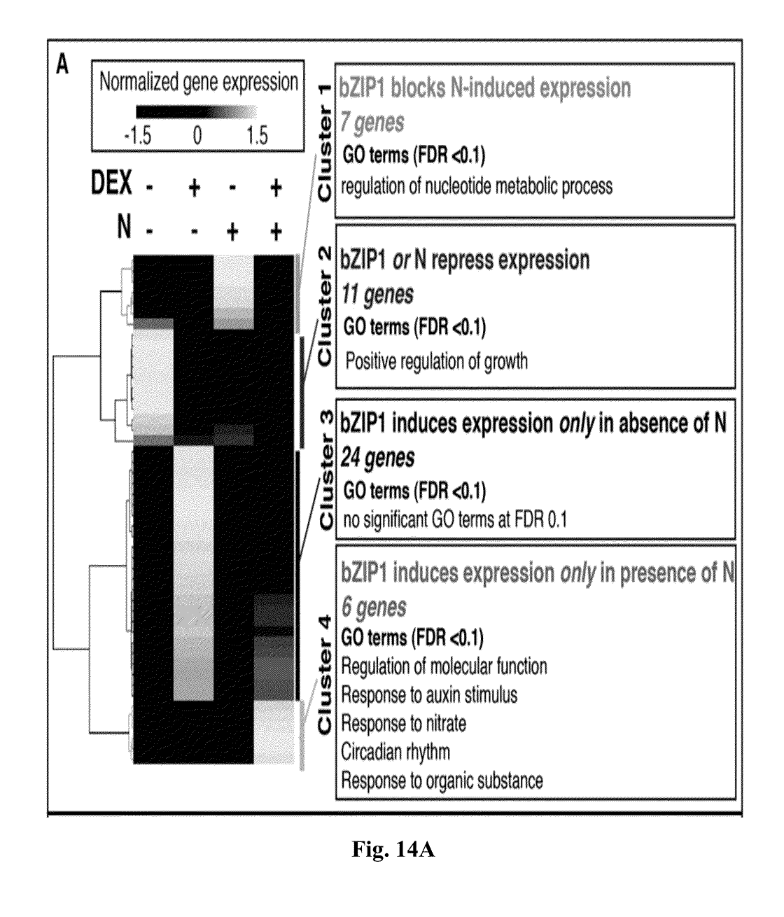

Transcriptome analysis using ATH1 Affymetrix Gene Chips was performed on cells transfected with 35S::GR-bZIP1 and subjected to the N, CHX and DEX treatments shown in FIG. 1C, in order to identify the primary targets regulated by bZIP1 in the context of the N-signal it transduces. ANOVA analysis identified 1,218 genes significantly regulated (FDR<0.05) in response to DEX-induced bZIP1 nuclear import (FIG. 10A; FIG. 10B; Table 4 and 5). 328 genes responded significantly to the N-signal in protoplasts, and show significant intersections with N-responses observed with a similar N-treatment (NH₄NO₃) and/or similar tissue (root) in planta (pval<0.001) (FIG. 13; Table 4) (Krouk et al., 2010, Genome biology 11:R123; Gutierrez et al., 2008, Proc. Natl. Acad. Sci. U.S.A. 105:4939; Palenchar et al., 2004, Genome Biology 5:R91; Gutierrez et al., 2007, Genome Biology 8:R7). With regard to signal perturbation, the N-responsive genes (328 genes) (FIG. 13) identified in the cell-based system, overlap significantly with the N-responsive genes identified from in planta studies (Krouk et al., 2010, Genome biology 11:R123; Gutierrez et al., 2008, Proc. Natl. Acad. Sci. U.S.A. 105:4939; Palenchar et al., 2004, Genome Biology 5:R91; Gutierrez et al., 2007, Genome Biology 8:R7) with a similar N-treatment (NH4NO3) and/or similar tissue (root) (pval<0.001 by Genesect) underscoring their in planta relevance. These N-responsive genes were also significantly enriched (pval=8.8E-13) with genes responsive to N across all root cell-types (Gifford et al., 2008, Proc. Natl. Acad. Sci. U.S.A. 105:803), suggesting the root protoplasts used in this study has an even representation of different root cell types.

TABLE 4
Genes identified by ANOVA and ChIP-Seq analysis.

| | |
|---|---|
| | Number |
| Category of Genes | of Genes |

Microarray Analysis

| | | |
|---|---|---|
| Significantly regulated | Nitrogen (FDR < 0.05) | 328 |
| by ANOVA factor | bZIP1* (FDR < 0.05) | 1218 |
| | NitrogenXbZIP1 (pval < 0.01) | 108 |
| | bZIP1* (FDR < 0.05) AND | 48 |
| | NitrogenXbZIP1* (pval < 0.01) | |

ChIP-SEQ Analysis

| | |
|---|---|
| bZIP1 bound genes* | 850 |

*genes considered as TF primary targets in this study.

TABLE 5

| | |
|---|---|
| Term | p-value |

A. Significantly over-represented GO terms in the DEX up-regulated genes (+CHX)

| | | |
|---|---|---|
| GO:0042221 | response to chemical stimulus | 1.75E−07 |
| GO:0050896 | response to stimulus | 1.75E−07 |
| GO:0009628 | response to abiotic stimulus | 2.22E−05 |
| GO:0009310 | amine catabolic process | 3.66E−05 |
| GO:0010033 | response to organic substance | 5.33E−05 |
| GO:0009063 | cellular amino acid catabolic process | 0.000127 |
| GO:0016054 | organic acid catabolic process | 0.000239 |
| GO:0046395 | carboxylic acid catabolic process | 0.000239 |
| GO:0009719 | response to endogenous stimulus | 0.000436 |
| GO:0006950 | response to stress | 0.000529 |
| GO:0009651 | response to salt stress | 0.000747 |
| GO:0044282 | small molecule catabolic process | 0.000899 |
| GO:0080167 | response to karrikin | 0.000899 |
| GO:0009725 | response to hormone stimulus | 0.00146 |
| GO:0006970 | response to osmotic stress | 0.00171 |
| GO:0009081 | branched chain family amino acid metabolic process | 0.00197 |
| GO:0009737 | response to abscisic acid stimulus | 0.00553 |

B. Significantly over-represented GO terms in the DEX down-regulated genes (+CHX)

| | | |
|---|---|---|
| GO:0050896 | response to stimulus | 8.89E−16 |
| GO:0006952 | defense response | 6.77E−12 |
| GO:0042221 | response to chemical stimulus | 6.77E−12 |
| GO:0006950 | response to stress | 1.19E−10 |
| GO:0010033 | response to organic substance | 5.79E−10 |
| GO:0051707 | response to other organism | 3.57E−09 |
| GO:0009607 | response to biotic stimulus | 1.37E−08 |
| GO:0051704 | multi-organism process | 1.37E−08 |
| GO:0010200 | response to chitin | 2.84E−08 |
| GO:0009620 | response to fungus | 1.24E−07 |
| GO:0031347 | regulation of defense response | 3.60E−07 |
| GO:0080134 | regulation of response to stress | 3.72E−07 |
| GO:0002376 | immune system process | 3.79E−06 |
| GO:0009743 | response to carbohydrate stimulus | 1.72E−05 |
| GO:0048583 | regulation of response to stimulus | 1.96E−05 |
| GO:0009719 | response to endogenous stimulus | 2.45E−05 |
| GO:0050832 | defense response to fungus | 2.95E−05 |
| GO:0009611 | response to wounding | 9.30E−05 |
| GO:0031348 | negative regulation of defense response | 0.000105 |
| GO:0045087 | innate immune response | 0.000151 |
| GO:0006955 | immune response | 0.000172 |
| GO:0009753 | response to jasmonic acid stimulus | 0.000241 |
| GO:0002682 | regulation of immune system process | 0.000326 |
| GO:0031408 | oxylipin biosynthetic process | 0.00076 |
| GO:0045088 | regulation of innate immune response | 0.00125 |
| GO:0050776 | regulation of immune response | 0.00125 |
| GO:0016310 | phosphorylation | 0.00135 |
| GO:0031407 | oxylipin metabolic process | 0.0014 |
| GO:0006468 | protein phosphorylation | 0.00169 |
| GO:0006793 | phosphorus metabolic process | 0.00194 |
| GO:0006796 | phosphate metabolic process | 0.00194 |
| GO:0009695 | jasmonic acid biosynthetic process | 0.0022 |
| GO:0008219 | cell death | 0.00326 |
| GO:0009694 | jasmonic acid metabolic process | 0.00326 |
| GO:0009725 | response to hormone stimulus | 0.00326 |
| GO:0009863 | salicylic acid mediated signaling pathway | 0.00326 |
| GO:0016265 | death | 0.00326 |
| GO:0050794 | regulation of cellular process | 0.00326 |
| GO:0071446 | cellular response to salicylic acid stimulus | 0.00326 |
| GO:0009737 | response to abscisic acid stimulus | 0.00331 |
| GO:0006334 | nucleosome assembly | 0.00467 |
| GO:0034728 | nucleosome organization | 0.00467 |
| GO:0010941 | regulation of cell death | 0.00486 |
| GO:0048584 | positive regulation of response to stimulus | 0.00497 |
| GO:0065004 | protein-DNA complex assembly | 0.00529 |
| GO:0071824 | protein-DNA complex subunit organization | 0.00529 |
| GO:0042742 | defense response to bacterium | 0.0057 |
| GO:0060548 | negative regulation of cell death | 0.0057 |
| GO:0045727 | positive regulation of translation | 0.00575 |
| GO:0009409 | response to cold | 0.00577 |
| GO:0031349 | positive regulation of defense response | 0.00577 |
| GO:0009751 | response to salicylic acid stimulus | 0.00661 |
| GO:0050789 | regulation of biological process | 0.00785 |
| GO:0010185 | regulation of cellular defense response | 0.00856 |
| GO:0010193 | response to ozone | 0.00856 |
| GO:0032270 | positive regulation of cellular protein metabolic | 0.00856 |
| | process | |
| GO:0051247 | positive regulation of protein metabolic process | 0.00856 |
| GO:0012501 | programmed cell death | 0.00886 |

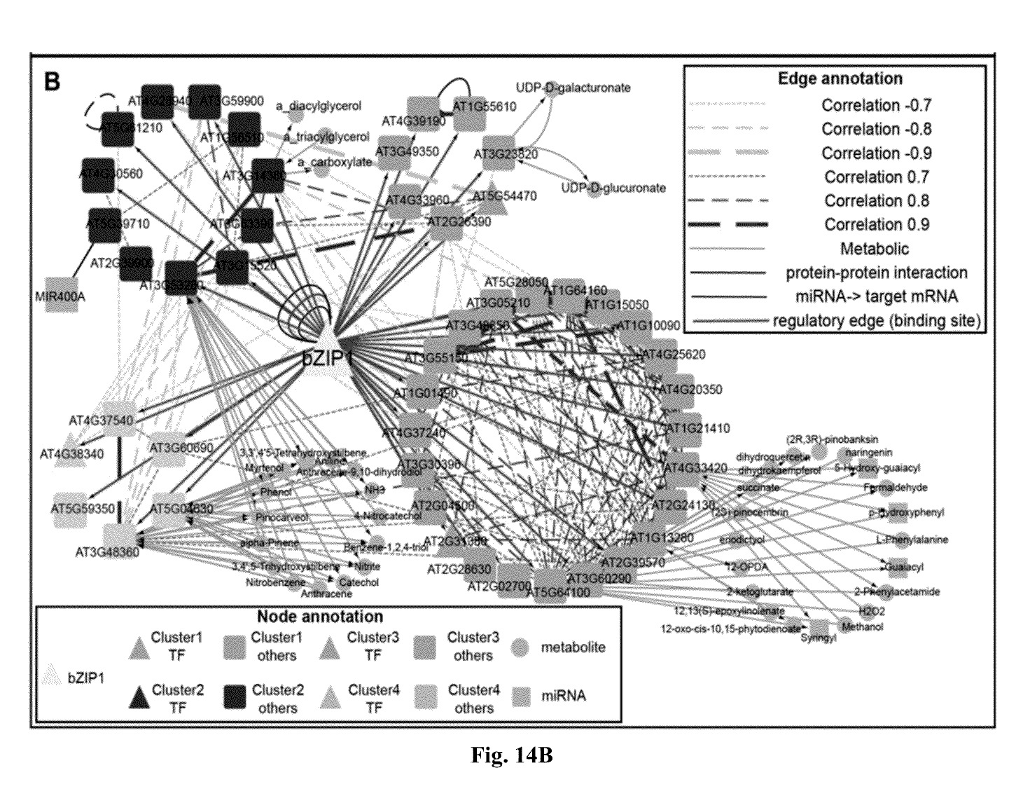

Forty-eight bZIP1 primary targets (FDR<0.05) were uncovered that show a significant TF×N-signal interaction (pval<0.01) (Table 6). These genes responding to bZIP1×N interactions form four distinct expression clusters (FIG. 14A) that can be viewed as a context-dependent bZIP1 GRN (FIG. 14B). Intriguingly, cluster 4 genes, whose induction is completely dependent on the bZIP1×N interaction, are enriched with N-regulated biological processes such as auxin stimulus, circadian, and response to organic substance (FIG. 14A). These 1,218 genes (including the 48 bZIP1×N responsive genes) are deemed to be primary targets of bZIP1, as gene responses to DEX-induced TF nuclear import were assayed in the presence of CHX, which blocks regulation of secondary targets controlled by other TFs downstream of bZIP1 (Bargmann et al., 2013, Molecular Plant 6(3):978). Thus, bZIP1 primary targets are expected to be regulated in response to TF perturbation under both +CHX and −CHX conditions. A significant overlap (pval<0.001) was observed between the bZIP1-regulated genes identified in +CHX samples and −CHX samples.

TABLE 6
Genes that are regualted by DEX (FDR < 0.05) and also regulated
by the interaction of N and DEX (pval < 0.01) forming 4 clusters
based on their expression patterns by Hierachical clustering in Mev

| | | |
|---|---|---|
| Locus | Symbol | Fullname |

A. Cluster 1

| | | |
|---|---|---|
| AT4G39190 | | |
| AT1G55610 | BRL1 | BRI1 like |
| AT3G49350 | | |
| AT3G23820 | GAE6 | UDP-D-glucuronate 4-epimerase 6 |
| AT4G33960 | | |
| AT5G54470 | BBX29 | B-box domain protein 29 |
| AT2G26390 | | |

B. Cluster 2

| | | |
|---|---|---|
| AT3G59900 | ARGOS | AUXIN-REGULATED GENE INVOLVED |
| | | IN ORGAN SIZE |
| AT5G39710 | EMB2745 | EMBRYO DEFECTIVE 2745 |
| AT4G28940 | | |
| AT4G30560 | ATCNGC9 | cyclic nucleotide gated channel 9 |
| AT3G15520 | | |
| AT1G56510 | ADR2 | ACTIVATED DISEASE RESISTANCE 2 |
| AT2G39900 | WLIM2a | WLIM2a |
| AT3G63390 | | |
| AT3G14360 | | |
| AT3G53280 | CYP71B5 | cytochrome p45 71b5 |
| AT5G61210 | ATSNAP33 | |

C. Cluster 3

| | | |
|---|---|---|
| AT2G04500 | | |
| AT3G05210 | ERCC1 | |
| AT3G30396 | | |
| AT1G13280 | AOC4 | allene oxide cyclase 4 |
| AT2G28630 | KCS12 | 3-ketoacyl-CoA synthase 12 |
| AT4G33420 | | |
| AT2G31380 | BBX25 | B-box domain protein 25 |
| AT3G60290 | | |
| AT2G02700 | | |
| AT5G64100 | | |
| AT4G37240 | | |
| AT4G20350 | | |
| AT1G64160 | AtDIR5 | |
| AT1G15050 | IAA34 | indole-3-acetic acid inducible 34 |
| AT1G10090 | | |
| AT1G13270 | MAP1B | METHIONINE AMINOPEPTIDASE 1B |
| AT3G55150 | ATEXO7H1 | exocyst subunit exo7 family protein H1 |
| AT3G48650 | | |
| AT2G39570 | ACR9 | ACT domain repeats 9 |
| AT2G24130 | | |
| AT5G28050 | | |
| AT4G25620 | | |
| AT1G21410 | SKP2A | |
| AT1G01490 | | |

D. Cluster 4

| | | |
|---|---|---|
| AT3G60690 | | |
| AT3G48360 | ATBT2 | |
| AT4G37540 | LBD39 | LOB domain-containing protein 39 |
| AT5G59350 | | |
| AT5G04630 | CYP77A9 | cytochrome P45, family 77, subfamily A, |
| | | polypeptide 9 |
| AT4G38340 | | |

To next identify primary bZIP1 targets whose promoter was bound by the GR-bZIP1 fusion protein either directly or indirectly through an interacting TF partner in a protein complex, a micro-ChIP protocol (Dahl et al., 2008, Nucleic Acids Research 36:e15) was adapted using anti-GR antibodies to pull down genomic regions bound to bZIP1 (FIG. 1C). Micro-ChIP and transcriptome data were derived from cells expressing 35S::GR-bZIP1 in parallel (FIG. 1C). Genic regions enriched in the ChIP DNA bound to GR-bZIP1 (peak seeding >=5 fold; extension >=2 fold) compared to the background (input DNA), were identified using the QuEST peak-calling algorithm (Valouev et al., 2008, Nature Methods 5:829) (FIG. 10A). This analysis identified 850 target genes with significant bZIP1 binding (FDR<0.05) (FIG. 10D), which includes several validated bZIP1 target genes (e.g. ASN1 and PropH) previously uncovered by ChIP-qPCR in planta (Dietrich et al., 2011, The Plant Cell 23:381-395).

It was confirmed that the 1,218 genes responding to bZIP1 perturbation and the 850 genes with significant binding to bZIP1 are enriched in bZIP1 primary targets by cis-regulatory motif analysis using MEME (Bailey et al., 2009, Nucleic Acids Research 37:W202) and elefinder (Li et al., 2011, Plant physiology 156:2124), which searches for known bZIP1 binding sites. Genes induced or bound by bZIP1 (644 genes) showed a highly significant overrepresentation of “G/C-box” (FIGS. 10 C&E), a cis-element previously shown to bind bZIP1 in vitro (Kang et al., 2010, Molecular Plant 3:361). A distinct bZIP-binding motif called the “GCN4 binding motif” (Onodera et al., 2001, The Journal of Biological Chemistry 276:14139) was significantly over-represented in the 574 genes repressed in response to bZIP1 perturbation (FIG. 10C). The GCN4 motif has been reported to mediate nitrogen and amino acid starvation sensing in both yeast and plants (Hill et al., 1986, Science 234:451; Muller et al., 1993, The Plant Journal: for cell and molecular biology 4:343), suggesting a functional conservation between bZIP1 and nutrient sensing. Lastly, the FORC^{A} motif, previously implicated in integrating light and defense signaling (Evrard et al., 2009. BMC Plant Biology 9:2), was shown to be over-represented in the 850 bZIP1 bound genes (FIG. 10E), consistent with the known role of bZIP1 in planta (Baena-Gonzalez et al., 2007, Nature 448:938; Kang et al., 2010, Molecular Plant 3:361; Hanson et al., 2007, The Plant Journal 53:935).

Identification of Temporal Modes of bZIP1 Primary Target Gene Regulation.

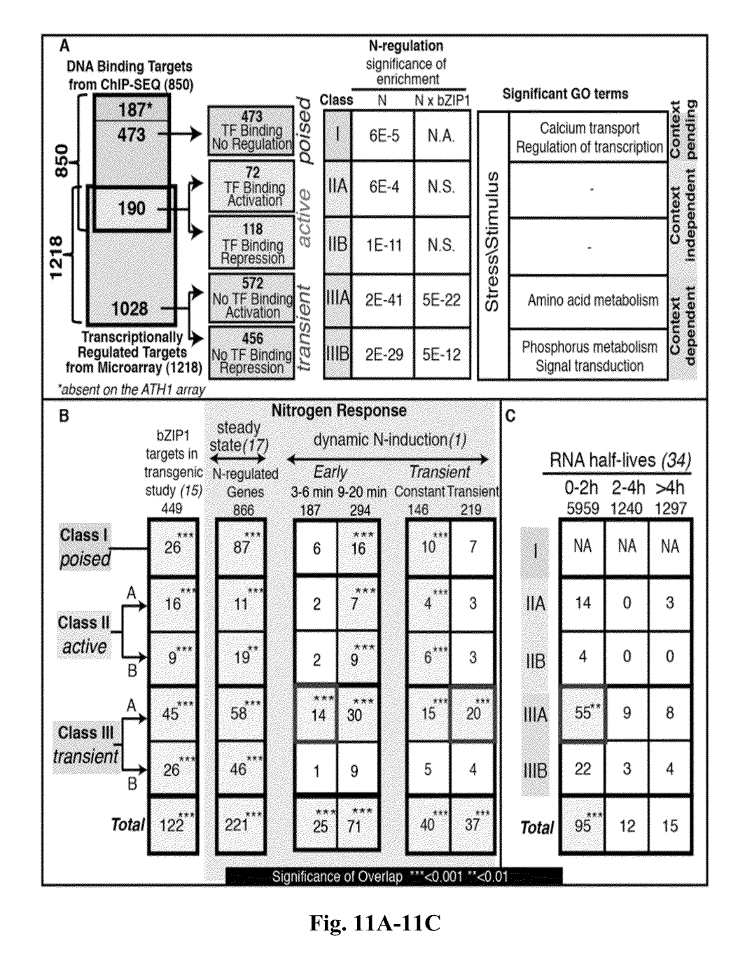

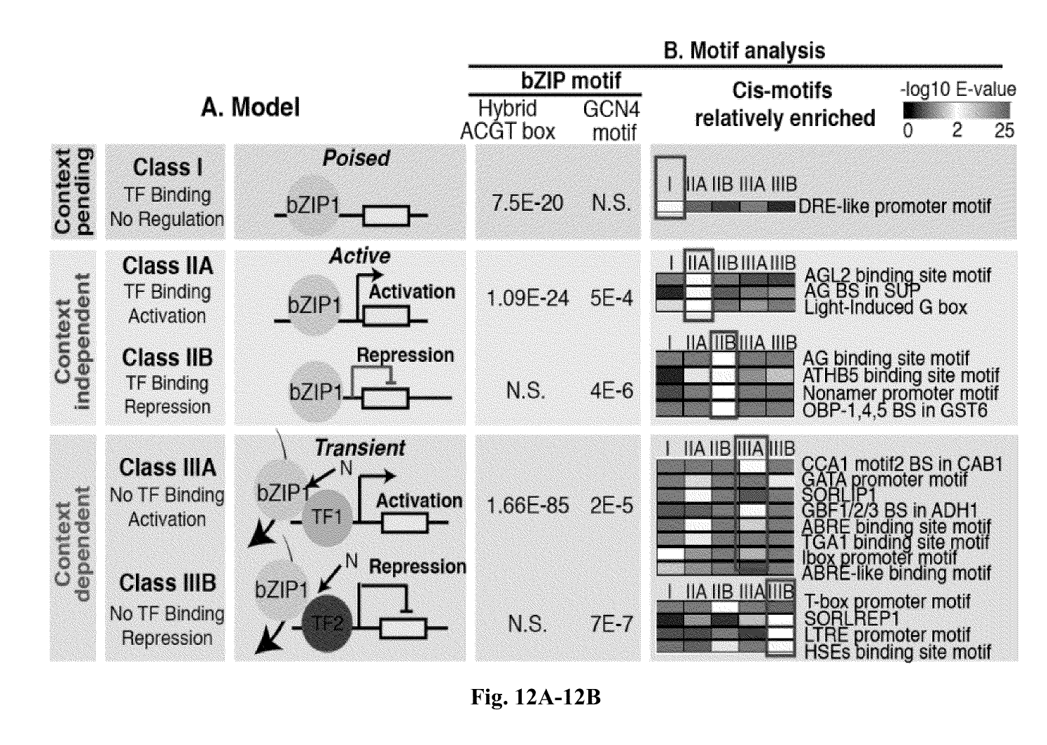

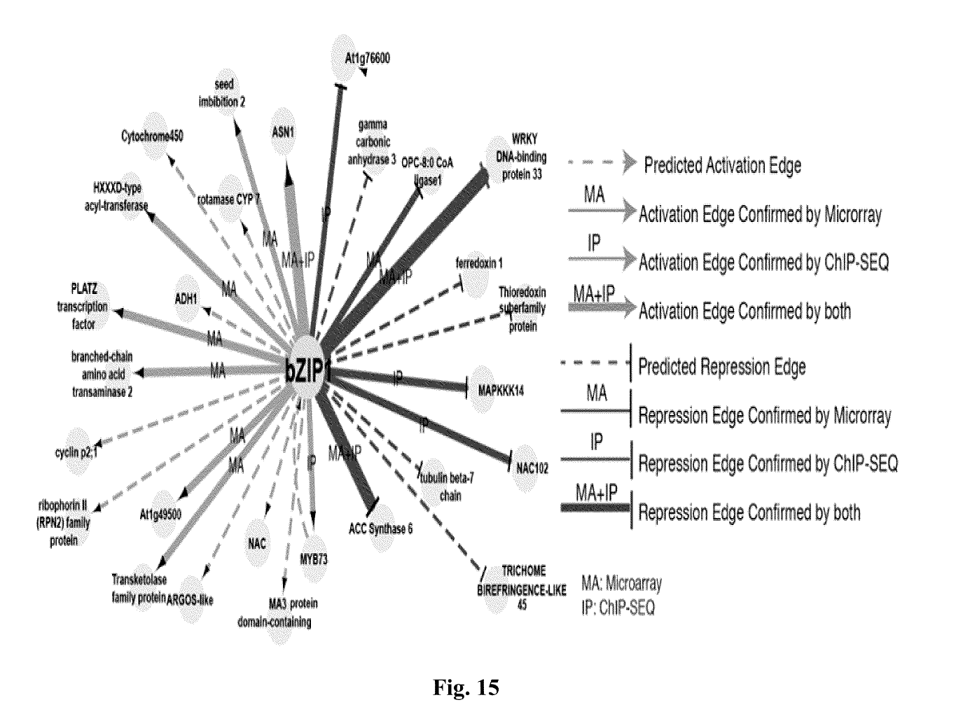

Mechanisms underlying temporal, signal-mediated modes of TF action were identified by integrating results from transcriptome and ChIP-Seq, and then performing analysis of signal context, biological function, and cis-element enrichment in bZIP1 primary target genes (FIG. 10A). bZIP1-regulated primary TF targets (1,218 genes) were compared with the bZIP1-bound TF-targets (663 out of 850 genes, because 187 are not on the ATH1 microarray) (FIG. 11A). This analysis identified three classes of primary TF targets (FIG. 11A) that represent distinct modes-of action for bZIP1: Class I: 473 genes with TF binding only; Class II: 190 genes that are TF bound and regulated; and Class III: 1,028 genes that are regulated by, but not bound to the TF (FIG. 11A). All three classes of bZIP1 primary targets are: i) enriched in known bZIP1 binding sites (FIG. 12B); ii) overlap significantly with genes previously shown to be regulated by bZIP1 from in planta studies (Kang et al., 2010, Molecular Plant 3:361; Gutierrez et al., 2008, Proc. Natl. Acad. Sci. U.S.A. 105:4939) (FIG. 11B; FIG. 15); iii) shared significant GO terms associated with known bZIP1 functions (e.g. Stimulus/Stress) (FIG. 11A); and iv) overlap with genes induced by carbon-starvation and darkness (Krouk et al., 2009, PLoS Computer Biology 5:e1000326) (FIG. 16), which is consistent with the known role of bZIP1 in planta (Baena-Gonzalez et al., 2007, Nature 448:938; Kang et al., 2010, Molecular Plant 3:361; Hanson et al., 2007, The Plant Journal 53:935). In addition to these common features, the three classes of bZIP1 primary target genes show distinguishing features.

In Planta Cross-Validation of the Three Classes of bZIP1 Primary Targets.

The in vivo relevance of all three classes of bZIP1 primary targets was validated based on comparison to targets identified in planta in i) a constitutive bZIP1 overexpression line (Kang et al., 2010, Molecular Plant 3:361) (122/449 genes; p-val<0.001) (FIG. 11B) and ii) predicted from an organic-N regulatory network (Gutierrez et al., 2008, Proc. Natl. Acad. Sci. U.S.A. 105:4939) (14/27 genes; p-val<0.001) (FIG. 15). Additionally, the potential relevance was determined for each bZIP1-target class in the signaling pathways previously associated with bZIP1 regulation in planta, including sugar (Kang et al., 2010, Molecular Plant 3:361) and light (Baena-Gonzalez et al., 2007, Nature 448:938). Intersections with genes repressed by carbon (C) and light (L) (Krouk et al., 2009, PLoS Computer Biology 5:e1000326) in roots and shoots (FIG. 16) were highly significant (p-val<0.001) across all three classes of bZIP1 primary targets identified. This result is consistent with previous reports that bZIP1 is a master regulator in response to light and sugar starvation (Weltmeier et al., 2008, Plant Molecular Biology 69:107; Baena-Gonzalez et al., 2007, Nature 448:938; Kang et al., 2010, Molecular Plant 3:361; Hanson et al., 2007, The Plant Journal 53:935).

Cis-Element Analysis of the Three Classes of bZIP1 Targets.

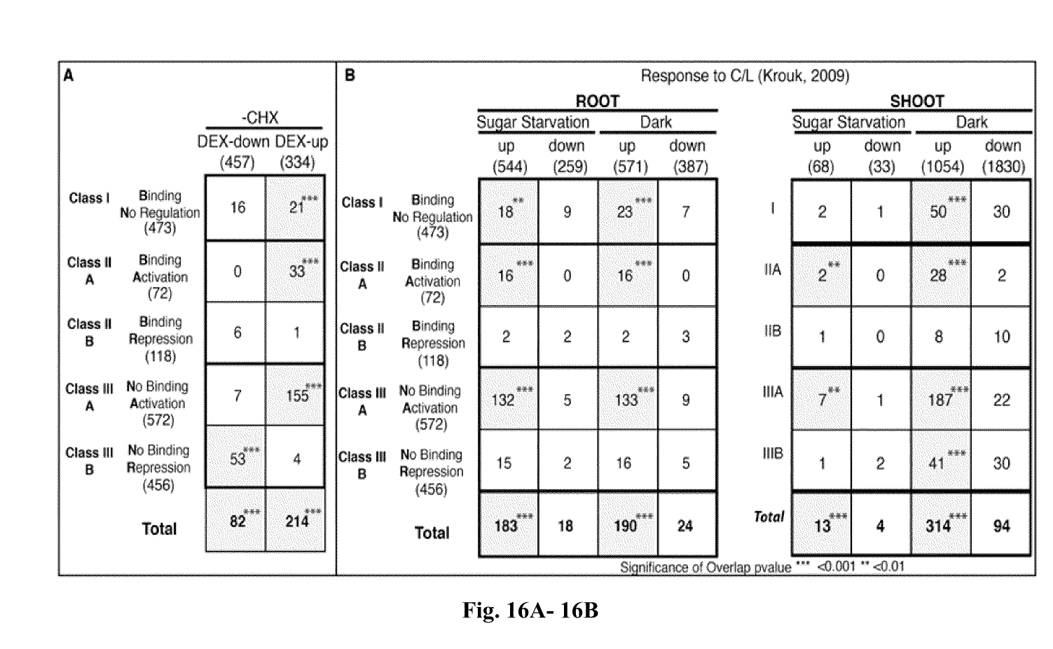

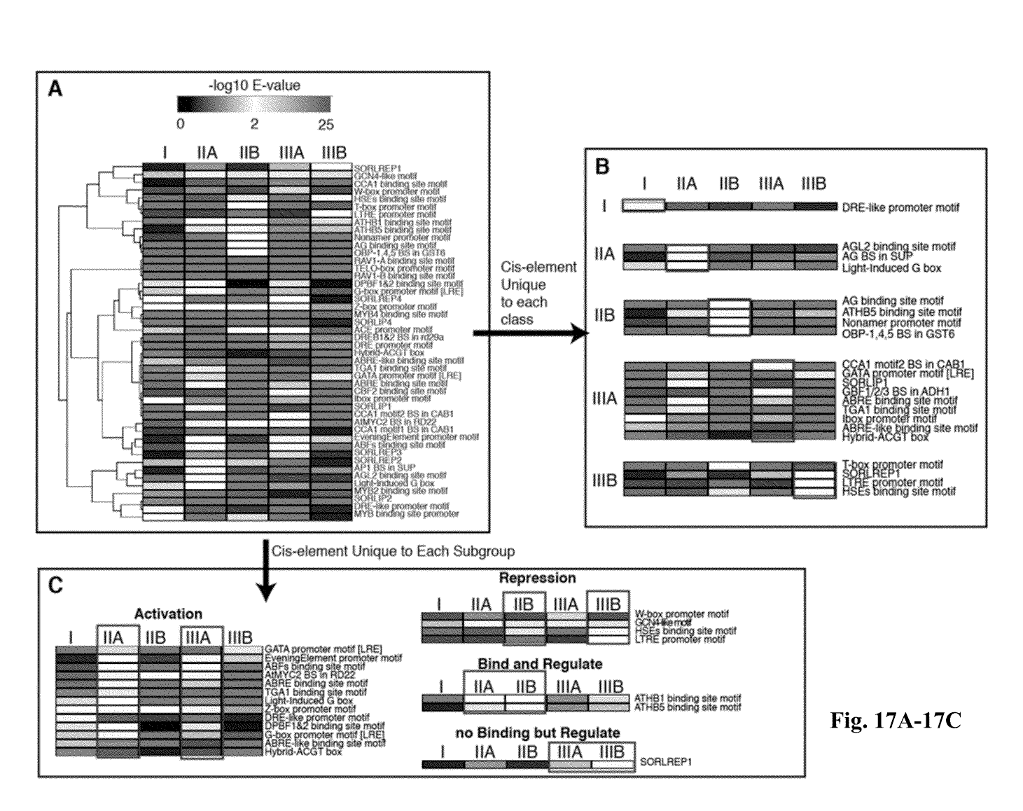

Cis-element analysis of each of the three subclasses of bZIP1 regulated gene targets show enrichment of known bZIP binding sites (FIG. 12B). Genes that either bind to bZIP1 or are activated by bZIP1 (Class I, IIA and IIIA), show significant over-representation of the known bZIP1 binding site “ACGT” box: including G-box, C-box or hybrid G/C-box (Kang et al., 2010, Molecular Plant 3:361) (FIG. 12B; FIG. 17). By contrast, genes that are repressed by bZIP1 do not have the canonical “ACGT” core, and instead posses the GCN4 binding motif for the bZIP family—as well as a W-box (FIG. 12B; FIG. 17). Interestingly, the GCN4 motif was reported to mediate nitrogen and amino acid starvation sensing in both yeast and plants (Onodera et al., 2001, The Journal of Biological Chemistry 276:14139; Hill et al., 1986, Science 234:451; Muller et al., 1993, The Plant Journal: for cell and molecular biology 4:343), suggesting a link between bZIP1 and nutrient sensing. A non-exclusive alternative interpretation is that bZIP1 may work with a WRKY family partner to repress primary target genes.

Class I “Poised” bZIP1 Targets:

TF Binding, No regulation. This class of bZIP1 primary targets were specifically and significantly overrepresented in genes involved in “regulation of transcription” and “calcium transport” (FDR<0.01) (FIG. 11A). These functions suggest that bZIP1 may serve as a master TF, that is bound to and “poised” to activate these downstream regulatory genes in response to a signal not provided in the experimental set-up, or that requires a TF partner not present in root cell protoplasts.

Class II “Active” bZIP1 Targets: TF Binding and Regulation.

The 190 primary bZIP1 target genes in Class II, represents a 29% overlap (p-val<0.001) between the transcriptome and ChIP-Seq data, which compares favorably to such overlaps in other TF studies in planta (23% ABI3 (Monke et al., 2012, Nucleic Acids Research 40:8240); 25% PIL5 (Oh et al., 2009, The Plant Cell Online 21:403)). Class II genes are the classical “gold standard” set that are the only primary targets identified in other TF studies that require TF-binding to define primary targets. For bZIP1, these primary targets in Class II have an overrepresentation in genes involved in “response to stress/stimulus” (FDR<0.01), which was a term common to all three classes of bZIP1 targets. No class-specific GO-terms were identified for these “classic” Class II bZIP1 primary target genes (FIG. 11A).

Class III “Transient” bZIP1 Targets: TF Regulation, but No Detectable TF Binding.

Unexpectedly, the Class III bZIP1 primary target genes, that are regulated by, but not detectably bound to the TF, turned out to be the largest set of bZIP1 primary target genes (1,028) detected in this study. The Class III genes were identified as primary bZIP1 targets based on gene regulation in response to the nuclear import of bZIP1 performed in the presence of CHX (to block activation of secondary targets), but were not detected in the parallel ChIP-Seq analysis to be bound by bZIP1 directly or indirectly in a protein complex containing bZIP1. In either scenario—direct binding of bZIP1 to its gene target or bZIP1 binding via interacting TF partners—the bZIP1 target gene should be detected by ChIP-Seq if the interaction is stable. This led to the hypothesis that the Class III primary bZIP1 target genes that are regulated in response to DEX-induced bZIP1 nuclear import may be the result of a transient TF-target association not detectable by ChIP-Seq at the time of sampling. A series of results supports this view, and also indicates that the Class III “transient” bZIP1 primary targets are most relevant to the function of bZIP1 in transducing the N-signal provided. First, the Class III “transient” bZIP1 primary target genes show a substantial (117/328) and the most significant overlap with N-responsive genes (FIG. 13) identified in the study (Class IIIA: pval=2e-41; Class IIIB: pval=2e-29) compared to Classes I and II (FIG. 11A). Second, out of the 48 primary targets regulated by bZIP1×N interaction (FIG. 14), 47 of these belong to Class III: Class IIIA (29 genes regulated by bZIP1×N interaction) (pval=5e-22) and Class IIIB (18 genes regulated by bZIP1×N interaction) (pval=5e-12) (FIG. 11A). This suggests that the bZIP1 regulation of Class III genes is likely modified by the N-signal, which may involve a post-translational modification of bZIP1 and/or by translational/transcription effects on its interacting partners (FIG. 1B). Third, only Class III bZIP1 primary targets showed a significant enrichment in genes involved in processes related to the N-signal including “amino acid metabolism”, “phosphorus metabolism” and “signal transduction” (FDR<0.01) (FIG. 11A). Lastly, but most importantly, only Class IIIA bZIP1 primary targets are specifically enriched with genes that respond to N in a transient and rapid manner in planta (FIG. 11B) (Krouk et al., 2010, Genome Biology 11:R123), as discussed in detail below.

Class III “Transient” bZIP1 Target Genes Show an Early and Transient N-Response in Planta.

To assess the significance of the three classes of bZIP1 targets identified in this cell-based system, the classes were compared to studies that have implicated bZIP1 as a master hub in mediating responses to N nutrient signals in planta (Gutierrez et al., 2008, Proc. Natl. Acad. Sci. U.S.A. 105:4939; Obertello et al., 2010, BMC Systems Biology 4:111). Indeed, all three classes of bZIP1 primary targets identified in this cell-based system were significantly enriched (pval<0.001) in genes regulated by an identical nitrogen treatment (NH₄NO₃) in an in planta study (FIG. 11B) (Gutierrez et al., 2008, Proc. Natl. Acad. Sci. U.S.A. 105:4939). The link between temporal N nutrient signaling and the bZIP1 “transient” mode of action was investigated by comparing all three Classes of bZIP1 primary targets to a fine-scale, time-series dataset that uncovered dynamic N-responsive genes in roots (Krouk et al., 2010, Genome Biology 11:R123). This analysis shows that only Class IIIA “transient” bZIP1 targets genes are rapidly and transiently regulated by nitrogen treatments in planta, as follows: i) Rapid N-induction: Only Class IIIA “transient” bZIP1 primary targets show a significant overlap (pval<0.001) with early nitrate-responsive genes induced within 6 minutes following N-treatment (Krouk et al., 2009, PLoS Computer Biology 5:e1000326) (FIG. 11B). ii) Transient N-induction: Only Class IIIA “transient” bZIP1 activated targets are distinguished by their significant overlap (pval<0.001) with genes that show a transient response to nitrate-induction in roots from the in planta time-course study (Krouk et al., 2010, Genome Biology 11:R123) (FIG. 11B). Specifically, 20 Class IIIA bZIP1 primary target genes (Table 1) are transiently N-induced in planta, and specific gene induction kinetics (3-20 min) are shown for three sample genes (AT2G43400, AT4G38490, and AT5G04310) (FIG. 11B). These data support the notion that a temporal relationship between bZIP1 and the Class IIIA “transient” primary target genes likely mediates an early and transient response to the N-signal.

Cis-Element Context Analysis Uncovers Elements Associated with Signal×TF Interactions.

A distinguishing feature of the Class III “transient” bZIP1 primary targets is their significant enrichment in genes responding to a bZIP1×N-signal interaction (FIG. 10A). This could be a result of i) the post-translational modification of bZIP1 and/or ii) the transcriptional or post-translational modification of its interactors in response to N-signaling (FIG. 1B; FIG. 12A). To uncover evidence for possible bZIP1 TF partners, the class-specific enrichment of cis-elements in the promoters of genes in each of the three bZIP1 primary target classes was examined (FIG. 12B). The Class III “transient” bZIP1 primary target genes contained the largest number and most highly significant enrichment of cis-motifs, compared to the other classes of bZIP1 targets (FIG. 12B; FIG. 17). Specifically, promoters of Class IIIA genes (primary targets activated by bZIP1, but no detectable bZIP1 binding) are significantly enriched with bZIP family TF binding sites (e.g. the TGA1 binding site (Yilmaz et al., 2011, Nucleic Acids Research 39:D1118), ABRE binding site (Yilmaz et al., 2011, Nucleic Acids Research 39:D1118), and GBF1/2/3 binding site (de Vetten et al., 1995, Plant Journal 7:589)). Other significant co-inherited cis-elements were specifically found in Class IIIA bZIP1 targets and include: MYB family TF binding sites (1-box (Yilmaz et al., 2011, Nucleic Acids Research 39:D1118) and CCA1 motif (Yilmaz et al., 2011, Nucleic Acids Research 39:D1118)), GATA promoter motif (Yilmaz et al., 2011, Nucleic Acids Research 39:D1118), and the light responsive motif SORLIP1 (Yilmaz et al., 2011, Nucleic Acids Research 39:D1118). These findings suggest that Class IIIA “transient” TF-target genes may be co-activated by bZIP1 and other TFs, including other bZIP family members, for which there is in vivo evidence of association with bZIP1 (Kang et al., 2010, Molecular Plant 3:361; Ehlert et al., 2006, The Plant Journal 46:890). For the Class IIIB bZIP1 target genes (primary target genes repressed by bZIP1, but no detectable bZIP1 binding), a number of cis-elements implicated in light and temperature signaling were significantly over-represented in their promoters, including T-box, SORLREP1, LTRE, and HSE binding site (Yilmaz et al., 2011, Nucleic Acids Research 39:D1118). Combined, the significant enrichment in Class III “transient” bZIP1 primary targets of genes i) early and ii) transiently regulated in response to a N-signal, iii) whose expression depends on a N×TF interaction, and iv) whose promoters are enriched in co-inherited cis-elements, support a model of temporal bZIP1-target association in response to the N-signal and/or a N-responsive interaction of bZIP1 with other TFs, as depicted in FIG. 12A.

### 7.4. Discussion and Concluding Remarks

A previously unrecognized “transient” mode of TF action was uncovered by a conceptual innovation in the experimental design to temporally perturb both a TF and signal, and in the integration and interpretation of TF-binding and TF-regulation data. This allowed for identification of primary TF targets based on either gene regulation or TF-binding, and the association of this regulation with a signal. This contrasts with previous studies of TFs in both plants and animals, where the identification of primary targets has been limited to TF-binding and/or the overlap between TF-regulation and TF-binding (Reeves et al., 2011, Plant Molecular Biology 75:347; Gorski et al., 2011, Nucleic Acids Research 39:9536; Hull et al., 2013, BMC Genomics 14:92; Fujisawa et al., 2011, Planta 235:1107; Wagner et al., 2004, The Plant Journal: for cellular and molecular biology 39:273). The approach enabled discovery of a new class of “transient” TF targets that are regulated by the TF but not detectably bound by it, because of three complementary features of the system: i) the ability to temporally induce the nuclear import of the TF bZIP1 in the presence or absence of a signal; ii) the use of a protein synthesis inhibitor (CHX) to identify primary TF-targets based solely on gene regulation; and iii) the ability to perform transcriptome analysis and ChIP-Seq on the same samples which allowed direct data comparison. Combining these features enabled the distinction between three temporal modes of bZIP1 action in regulating primary TF-target genes: “poised”, “active” and “transient”. By examining the TF modes of action in the presence or absence of a signal it transduces (N), it was found that Class III “transient” gene targets (TF-regulated but not bound) were most relevant to the N-signal provided, as they show unique and significant: i) enrichment in N-responsive genes (FIG. 11A), ii) early and iii) transient induction by a N-signal (FIG. 11B), iv) regulation by TF×N-signal interactions (FIG. 11A), and v) GO-term enrichment in N-related processes (FIG. 11A). These features distinguish the Class III “transient” TF-target genes, compared to the other two classes of primary TF targets: “poised” and “active”. It is noteworthy that the Class III “transient” TF-targets identified in the cell-based system also play an important role in vivo—based on significant overlap with in planta data (FIG. 11B). However, they would have been dismissed as secondary TF-targets in those in planta studies, and their role in mediating a dynamic GRN would have been missed.

This discovery suggests that the Class III “transient” TF-target genes are likely the result of a temporal association between bZIP1 with these targets, acting either directly on the primary target DNA and/or through TF partner interactions (FIG. 12A). In support of the role of TF partners in this temporal, N-signal mediated regulation, cis-element analysis revealed that the Class III “transient” bZIP1 target genes had the highest enrichment, both in number and in significance, of cis-elements that co-occurred with the bZIP1 binding site, compared to the inactive “poised” Class I genes and the constitutively “active” Class II genes (FIG. 12B). TFs associated with these co-occurring cis-elements include other bZIP family members and TFs belonging to the MYB family. Querying a protein-protein interaction database (Katari et al., 2010, Plant physiology 152:500) revealed that bZIP1 interacts with 11 other members of the bZIP family (Table 7). Interestingly, 3 out of these 11 bZIP TFs shown to interact with bZIP1 in vitro (Katari et al., 2010, Plant physiology 152:500), were also determined to be primary targets of bZIP1 in this study (bZIP25, bZIP53, bZIP9), suggesting that bZIP1 regulates and activates some of its protein-interaction TF partners. The interactions between bZIP1 with bZIP25/53/9 have also been independently experimentally validated in vivo (Baena-Gonzalez et al., 2007, Nature 448:938; Kang et al., 2010, Molecular Plant 3:361; Ehlert et al., 2006, The Plant Journal 46:890). These data support the hypothesis that bZIP1 may be a master response gene that activates and interacts with specific bZIP family members, and/or potentially with members of the MYB family, to “temporally” co-regulate downstream genes in response to a N-signal.

TABLE 7
bZIP1 protein-protein interaction partners.

| | |
|---|---|
| At5g37780 | ACAM-1, CAM1, TCH1, calmodulin 1 |
| At1g66410 | ACAM-4, CAM4, calmodulin 4 |
| At5g21274 | ACAM-6, CAM6, calmodulin 6 |
| At2g41100 | ATCAL4, TCH3, Calcium-binding EF hand family protein |
| At3g51920 | ATCML9, CAM9, CML9, calmodulin 9 |
| At2g41090 | Calcium-binding EF-hand family protein |
| At3g43810 | CAM7, calmodulin 7 |
| At4g14640 | CAM8, calmodulin 8 |
| At5g41910 | MED10A, Mediator complex, subunit Med10 |
| At4g34590 | ATB2, AtbZIP11, BZIP11, GBF6, G-box binding factor 6 |
| At5g49450 | AtbZIP1, bZIP1, basic leucine-zipper 1 |
| At4g02640 | ATBZIP10, BZO2H1, bZIP transcription factor family |
| | protein |
| At2g18160 | ATBZIP2, bZIP2, GBF5, basic leucine-zipper 2 |
| At3g54620 | ATBZIP25, BZIP25, BZO2H4, basic leucine zipper 25 |
| At1g59530 | ATBZIP4, bZIP4, basic leucine-zipper 4 |
| At3g30530 | ATBZIP42, bZIP42, basic leucine-zipper 42 |
| At1g75390 | AtbZIP44, bZIP44, basic leucine-zipper 44 |
| At3g62420 | ATBZIP53, BZIP53, basic region/leucine zipper motif 53 |
| At1g13600 | AtbZIP58, bZIP58, basic leucine-zipper 58 |
| At5g28770 | AtbZIP63, BZO2H3, bZIP transcription factor family |
| | protein |
| At5g24800 | ATBZIP9, BZIP9, BZO2H2, basic leucine zipper 9 |

To place these findings in perspective, the general field of GRN validation has focused on determining when and how TF binding does, or does not, result in gene activation (Reeves et al., 2011, Plant Molecular Biology 75:347; Gorski et al, 2011, Nucleic Acids Research 39:9536). This focus has limited the field to studying the more stable and static “gold standard” interactions exemplified by the bZIP1 Class II genes (TF-bound and regulated). The discovery of the Class III “transient” TF-targets (TF-regulated, no binding) now opens the opposite question/perspective in the general field of transcriptional control: How and why can TF-induced changes in mRNA occur in the absence of stable TF binding? The simple explanation that the Class IIIA mRNA is stabilized by CHX or bZIP1 is not supported by the data, as +/−CHX results are comparable (FIG. 16), and there was no evidence for either bZIP1 regulated small RNAs or 3′ UTR elements that could affect RNA stability in Class III genes. Therefore, these transient TF-target interactions may be conceptualized as the “hit-and-run” model of transcription, which posits that a TF can act as a trigger to organize a stable transcriptional complex, after which transcription by RNA polymerase II can continue without the TF being bound to the DNA (Schaffner, 1988, Nature 336:427-428).

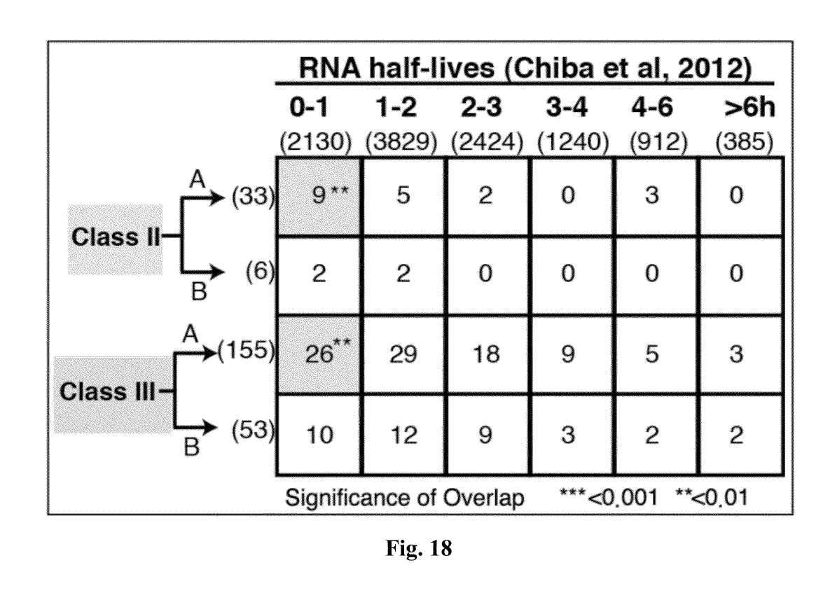

In support of this “hit-and-run” model, the Class III “transient” genes are enriched in mRNAs with short half-lives (<2 hour) (Chiba et al., 2013, Plant & cell physiology 54:180) indicating that they are actively transcribed at the 5 hour time-point when the gene is induced by the TF but is not stably bound to it (FIG. 18). This “hit-and-run” model of TF action suggests a general mechanism for the deployment of an acute response to nutrient level change, in which a master regulatory TF transiently and rapidly activates a large set of genes in response to a signal. This “pioneer” TF responds to N-signals possibly by recruiting TF partners, as supported by the finding that Class III targets are most significantly enriched with cis-regulatory elements of known bZIP1 interactors.

The “transient”, signal-induced association of a target with a TF can be analaogized to a “touch-and-go” (hit-and-run) landing or circuit maneuver used in aviation. This involves landing a plane on a runway and taking off again without coming to a full stop, allowing many landings in a short time. This maneuver also allows pilots to rapidly detect or avoid another plane or object on the runway, and could serve an analogous role for bZIP1 and its TF partners. The “touch-and-go” (hit-and-run) mode may enable bZIP1 to “direct”, “detect” or “avoid” TFs on a gene target, or alternatively to rapidly activate and leave the promoter “empty” for its TF partners to occupy. By contrast, the more traditional “stop-and-go” action requiring a full stop before taking off again, is a more stable maneuver which can be analogized to the classic Class II “gold standard” set, in which the TF lands (stably binds) and regulates a gene. While these more stable and static interactions have been the focus of most TF studies, the discovery of this new “touch-and-go” (hit-and-run) mode of TF action opens a new concept and field of inquiry in the study of dynamic GRNs in plants and animals.

### 8. EXAMPLE 3

### 8.1. Plant Growth and Treatment

Rice seeds (Oryza sativa ssp. japonica) were kindly provided by Dale Bumpers of the National Rice Research Center (AR, USA). Seeds were surface-sterilized and vernalized on 1× Murashige and Skoog (MS) basal salts (custom-made; GIBCO) with 0.5 mM ammonium succinate and 3 mM sucrose, 0.8% BactoAgar at pH 5.5 for 3 days in dark conditions at 27° C. Germinated seeds were transferred to a hydroponic system (Phytatray II, Sigma Aldrich) containing basal MS salts (custom-made; GIBCO) with 0.5 mM ammonium succinate and 3 mM sucrose at pH 5.5 to grow for 12 days under long-day (16 h light: 8 h dark) at 27° C., at light intensity of 180 μE·s⁻¹·m⁻². Media was replaced every 3 days and the plants were transferred to fresh media containing basal MS salts for 24 h prior treatment. On day 13, plants were transiently treated for 2 h at the start of their light cycle by adding Nitrogen (N) at a final concentration of 20 mM KNO₃ and 20 mM NH₄NO₃ (referred here as lxN). Control plants were treated with KCl at a final concentration of 20 mM. After treatment, roots and shoots were harvested separately using a blade, and immediately submerged into liquid nitrogen and stored at −80° C. prior to RNA extraction.

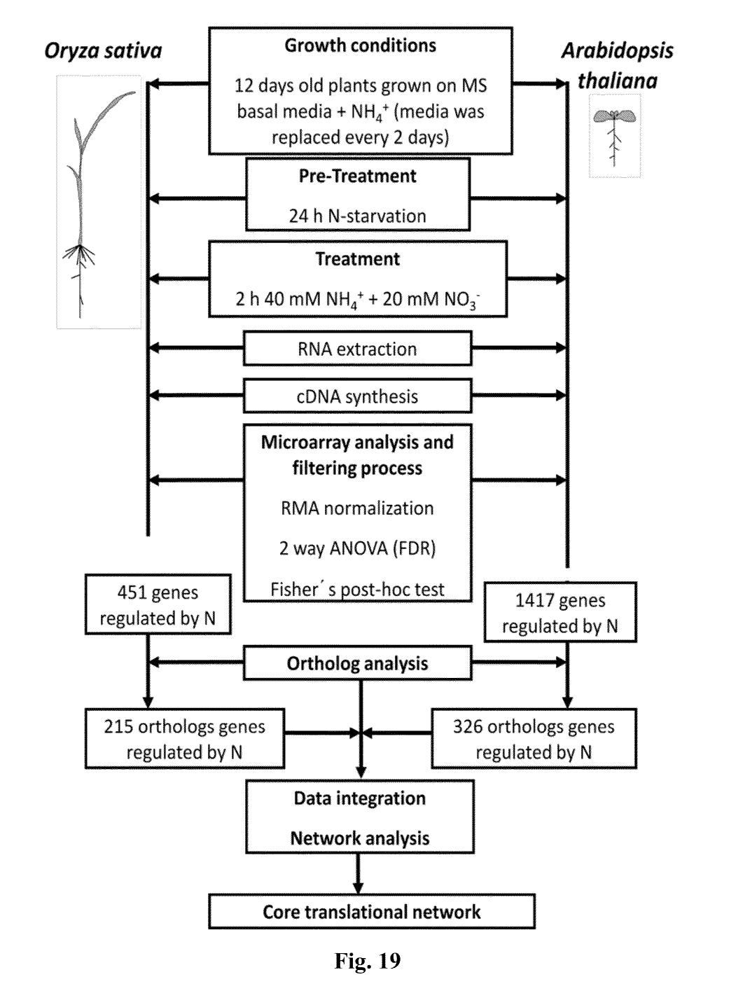

Arabidopsis seeds were placed for 2 days in the dark at 4° C. to synchronize germination. Seeds were surface-sterilized and then transferred to a hydroponic system (Phytatray I, Sigma Aldrich) containing the same media previously described for rice (pH 5.7). Growth conditions were the same as in rice, except that plants were under 50 μE·s-1·m-2 light intensity at 22° C. N-starvation and treatments were done as described above (FIG. 19). RNA was isolated using TRIzol reagent following manufacturer's protocols.

### 8.2. Microarray Experiments and Analysis

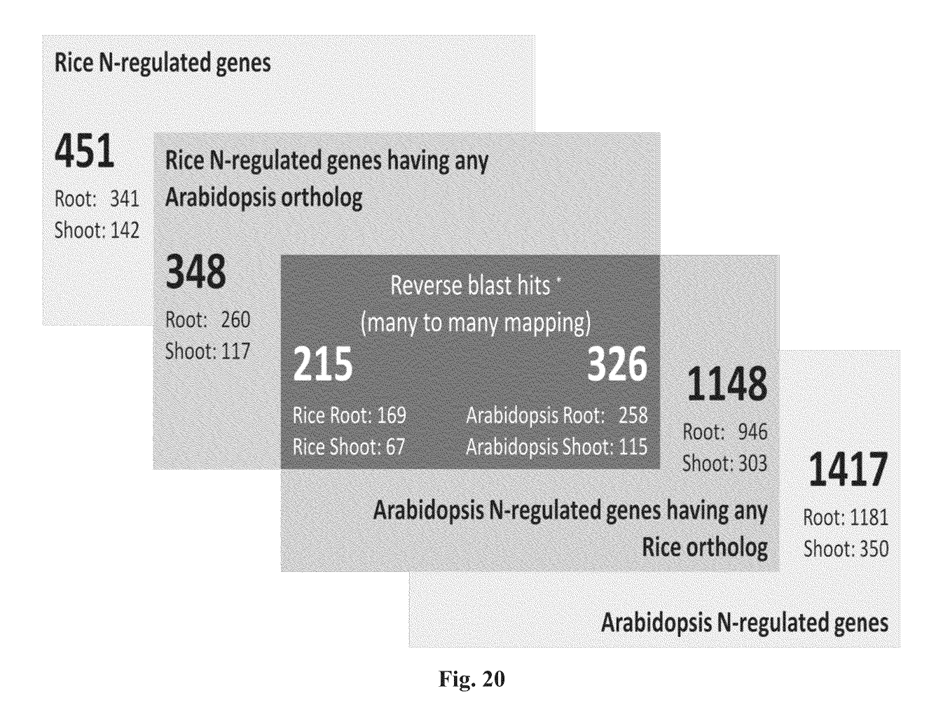

cDNA synthesis, array hybridization and normalization of the signal intensities were performed according to the instructions provided by Affymetrix. Affymetrix Arabidopsis ATH1 Genome Array Chip and Rice Genome Array Chip were used for respective species. Data normalization was performed using the RMA (Robust Microarray Analysis) method in the Bioconductor package in R statistical environment. A two-way Analysis of Variance (ANOVA) was performed using custom-made function in R to identify probes that were differentially expressed following N treatment. The p-values for the model were corrected for multiple hypotheses testing using FDR correction at 5% (Benjamini and Hochberg, 1995, Journal of the Royal Statistical Society 57:289). The probes passing the cut-off (p≦0.05) for the model and, N treatment or interaction of N treatment and tissue, were deemed significant. A Tukey's HSD post-hoc analysis was performed on significant probes to determine the tissue specificity of N-regulation at p-value cut-off ≦0.05 and fold-change ≧1.5-fold (FIG. 19). Affy probes mapping to more than one gene were disregarded resulting in a significant set of N-regulated 1417 Arabidopsis genes and 451 Rice genes (FIG. 20).

Orthologous N-regulated genes between Rice and Arabidopsis were obtained using reverse Blast (Camacho et al., 2008, BMC Bioinformatics 10:421) with an e-value ≦1e⁻²⁰, thereby allowing for multiple ortholog hits (FIG. 20).

### 8.3. Network Analysis

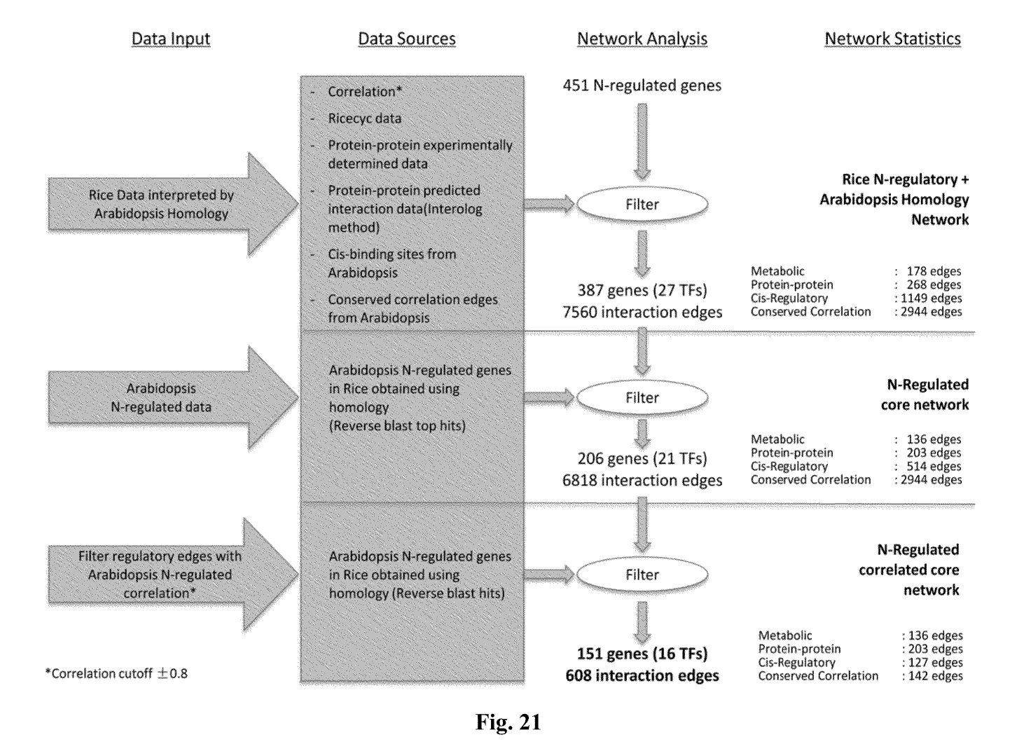

A Rice Multinetwork was generated using the following interactions (FIG. 21):

Metabolic interactions were obtained from RiceCyc (Dharmawardhana et al., 2013, Rice 6:15).

Protein-Protein interactions were obtained from the PRIN database (Gu et al., 2011, BMC Bioinformatics 12:161), and published work, which include experimentally determined and computationally predicted interactions (Ding et al., 2009, Plant Physiology 149(3):1478; Rohila et al., 2006, The Plant Journal 46:1; Ho et al., 2012, The Rice Journal 5:15).

Predicted Regulatory interactions were created between a Transcription Factor (TF) and its putative target using TF family membership obtained from Grassius (Yilmaz et al., 2009, Plant Physiology 149:171) and identification of cis-regulatory motifs, obtained from AGRIS (Palaniswamy et al., 2006, Plant Physioloy 140:818), in 1000 bp upstream of promoter sequence of Target genes. Motifs were searched using the DNA pattern search tool from the RSA tools server with default parameters (van Helden, 2003, Nucleic Acids Research 31:3593).

The 451 N-regulated rice genes were queried against the Rice Multinetwork to create a N-regulated gene network in Rice. Additionally, conserved correlation edges between two N-regulated Rice genes were proposed if the respective Arabidopsis N-regulated orthologs were also correlated significantly in the same direction (both positively or negatively) with Pearson correlation coefficient ≧0.8. Predicted regulatory interactions were further restricted to those TF and Target pairs where the two were also significantly correlated (Pearson correlation coefficient ≧0.8 and p-value ≦0.01), which resulted in a network of 206 Rice genes, of which 21 are transcription factors, with 6,818 edges (FIG. 21).

The network was further refined by removing conserved correlation edges that are not supported with predicted regulatory edges which resulted in a “N-regulated correlated network” containing 151 Rice genes, of which 16 were TFs (Table 8). All network visualizations were created using Cytoscape (v2.8.3) software (Shannon et al., 2003, Genome Research 13:2498).

TABLE 8
Number of targets of transcription factors at each step in the network creation process.

| | | | | Rice Core |
|---|---|---|---|---|
| | | Rice Homology | Rice Core | Correlated |
| | | Network | Network | Network |
| Gene Locus ID | Gene Description | # of targets | # of targets | # of targets |
| LOC_OS01G54020 | transcription factor HBP-1b, putative, expressed | 210 | 114 | 41 |
| LOC_OS01G64000 | ABA response element binding factor, putative, | 136 | 79 | 34 |
| | expressed | | | |
| LOC_OS06G41100 | TGA10 transciption factor, putative, expressed | 114 | 48 | 5 |
| LOC_OS09G35030 | sbC8F6, putative, expressed | 108 | 64 | 1 |
| LOC_OS01G06640 | DNA binding protein, putative, expressed | 106 | 58 | 20 |
| LOC_OS05G37170 | transcription factor TGA6, putative, expressed | 86 | 51 | 9 |
| LOC_OS04G42950 | DNA binding factor 6, putative, expressed | 84 | 36 | 6 |
| LOC_OS02G06910 | auxin response factor 6, putative, expressed | 68 | | |
| LOC_OS04G55970 | DNA binding protein, putative, expressed | 63 | 30 | 4 |
| LOC_OS03G04310 | DNA binding protein, putative, expressed | 52 | 20 | 4 |
| LOC_OS06G07030 | dehydration responsive element binding protein, | 45 | | |
| | putative, expressed | | | |
| LOC_OS09G26420 | ethylene response factor, putative, expressed | 30 | | |
| LOC_OS08G42550 | AP2 domain containing protein, expressed | 26 | 9 | 1 |
| LOC_OS01G34060 | DNA binding protein, putative, expressed | 14 | 5 | 2 |
| LOC_OS05G38140 | bHLH transcription factor, putative, expressed | 6 | | |
| LOC_OS03G47730 | knotted1-interacting protein, putative, expressed | 3 | | |

A comparison of the number of TF targets at various network building steps as shown in FIG. 21, demonstrates that TFs with the most targets are more likely to be conserved between Arabidopsis and Rice and therefore are candidates for further translational studies (Table 9). BioMaps (GO-term enrichment analysis) of the targets of all TFs present in the “N-regulated core network” revealed that targets of only two TFs, LOC_Os01g64000 and LOC_Os01g64020, are enriched for “nitrate assimilation” and “nitrate metabolic process” (Table 10). A closer look at the N-assimilation pathway in the N-regulated Core Network revealed a set of 7 Rice transcription factors, which are directly targeting the genes in the N-assimilation pathway (Table 11). Three of the 7 TFs were also present in the correlated core N-regulated network, which implies that these TF-target gene pairs have conserved N-response in both Arabidopsis and Rice (Table 11).

TABLE 9
Rice and Arabidopsis orthologous transcription factors in the “N-regulated core network.”

| | | | | Arabidopsis Orthologs | |
|---|---|---|---|---|---|
| Rice Core Network | | | | Orthologs | |
| Gene Locus ID | Hubs | Rank | Gene Description | Gene ID | Orthologs Gene Description |
| LOC_OS01G64020 | 114 | 1 | transciption factor HBP-1b, | AT1G22070 | TGA3, TGA1A related gene 3 |
| | | | putative, expressed | AT1G77920 | bZIP transcription factor family protein |
| | | | | AT5G10030 | OBF4, TGA4, TGACG motif binding factor 4 |
| | | | | AT5G65210 | TGA1, bZIP transcription factor family protein |
| LOC_OS01G64000 | 79 | 2 | ABA response element binding | AT3G56850 | AREB3, DPBF3, ABA-responsive element binding protein 3 |
| | | | factor, putative, expressed | | |
| LOC_OS09G35030 | 64 | 3 | sbCBF6, putative, expressed | AT1G63030 | ddf2, Integrase type DNA-binding superfamily protein |
| LOC_OS01G05640 | 58 | 4 | DNA binding protein, putative, | AT3G25710 | ATAIG1, BHLH32, TMO5, basic helix-loop helix 32 |
| | | | expressed | | |
| LOC_OS05G17170 | 51 | 5 | transcription factor TGA6, | AT1G22070 | TGA3, TGA1A related gene 3 |
| | | | putative, expressed | AT3G77920 | bZIP transcription factor family protein |
| | | | | AT5G10030 | OBF4, TGA4, TGACG motif-binding factor 4 |
| | | | | AT5G65210 | TGA1, bZIP transcription factor family protein |
| LOC_OS06G41100 | 48 | 6 | TGA10 transciption factor, | AT1G22070 | TGA3, TGA1A-related gene 3 |
| | | | putative, expressed | AT1G77920 | bZIP transcription factor family protein |
| | | | | AT5G10030 | OBF4, TGA4, TGACG motif-binding factor 4 |
| | | | | AT5G65210 | TGA1, bZIP transcription factor family protein |
| LOC_OS04G42950 | 36 | 7 | DNA binding protein, putative, | AT5G26660 | ATMY886, MY886, myb domain protein 85 |
| | | | expressed | AT5G60890 | ATM834, ATR1, MY834, myb domain protein 34 |
| | | | | AT5G61420 | AtMY828, HAG1, MY828, P84G1, myb domain protein 28 |
| LOC_OS04G53970 | 30 | 8 | DNA binding protein, putative, | AT1G79700 | Integrase-type DNA-binding superfamily |
| | | | expressed | | proteinANTEGUMENTA-like 6 |
| | | | | AT2G23550 | RAP2.7, TOE1, related to AP2.7 |
| | | | | AT3G20840 | PLT1, integrase-type DNA binding superfamily protein |
| | | | | AT3G54320 | ASML1, ATWRI1, WRI, WRI1, integrase-type DNA |
| | | | | | binding superfamily protein |
| | | | | AT5G10510 | AIL6, PLT3, ANTEGUMENTA-like 6 |
| LOC_OS03G04310 | 20 | 9 | DNA binding protein, putative, | AT3G26744 | ATICE1, ICE1, SCRM, basic helix-loop-helix [bHLH] |
| | | | expressed | | DNA-binding superfamily protein |
| LOC_OS08G42950 | 9 | 10 | AP2 domain containing protein, | AT1G29200 | O fucosyltransferase family protein |
| | | | expressed | | |
| LOC_OS01G34060 | 5 | 11 | DNA binding protein, putative, | AT1G49010 | Duplicated homeodomain-like superfamily protein |
| | | | expressed | AT3G15350 | Homeodomain-likesuperfamily proteom |

TABLE 10
BioMaps (Gene Ontology Enrichment Analysis) of N-regulated TF targets in the “N-regulated
Core Network.” Only LOC_OS01G64020 and LOC_OS01G64000 targets had over-represented
GO-terms (“nitrate metabolic process” and “nitrate assimilation”) (p-value cutoff ≦ 0.05).

| | | # of Targets in | |
|---|---|---|---|
| | | Rice Core Network | |
| | | (N-assimilation | Over-represented |
| Gene Locus ID | Gene Description | pathway) | GO:Terms for targets |
| LOC_OS01G64020 | transcription factor HBP-1b, | 114 (5) | nitrate metabolic process |
| | putative, expressed | | (GO:0042126), nitrate |
| | | | assimilation (GO:0042128) |
| LOC_OS01G64000 | ABA response element | 79 (4) | nitrate metabolic process |
| | binding factor, putative, | | (GO:0042126), nitrate |
| | expressed | | assimilation (GO:0042128) |

TABLE 11
Rice and Arabidopsis orthologus transcription factors targeting the “N-assimilation pathway.”

| Rice TF Targets | | Rice Tfs | |
|---|---|---|---|
| ID | Description | ID | Description |
| LOC_OS01G25484 | ferrodoxin-nitrite reductase, chloroplast | LOC_OS01G64020 | transcription factor HBP-1b, putative |
| | precursor, putative, expressed | | expressed |
| LOC_OS01G48960 | glutamate synthase, chloroplast precursor, | | |
| | putative expressed | | |
| LOC_OS02G53130 | nitrate reductase, putative expressed | | |
| LOC_OS03G13250 | peptide transporter PTR2, putative, expressed | | |
| LOC_OS06G15370 | peptide transporter PTR2, putative, expressed | | |
| LOC_OS08G15379 | peptide transporter PTR2, putative, expressed | LOC_OS01G64000 | ABA response element binding factor, |
| LOC_OS01G25484 | ferrodoxin-nitrite reductase, chloroplast | | putative, expressed |
| | precursor, putative, expressed | | |
| LOC_OS14G48960 | glutamate synthase, chloroplast precursor, | | |
| | putative expressed | | |
| LOC_OS02G53130 | nitrate reductase, putative expressed | | |
| LOC_OS01G25484 | ferrodoxin-nitrite reductase, chloroplast | LOC_OS09G35030 | dbC8F6, putative, expressed |
| | precursor, putative, expressed | | |
| LOC_OS01G48960 | glutamate synthase, chloroplast precursor, | | |
| | putative expressed | | |
| LOC_OS03G15250 | peptide transporter PTR2, putative, expressed | | |
| LOC_OS08G15370 | peptide transporter PTR2, putative, expressed | LOC_OS01G05940 | DNA binding protein, putative, |
| LOC_OS01G48960 | glutamate synthase, chloroplast precursor, | | expressed |
| | putative expressed | | |
| LOC_OS01G48960 | glutamate synthase, chloroplast precursor, | LOC_OS05G37170 | transcrption factor TGAS putative, |
| | putative expressed | | expressed |
| LOC_OS03G13250 | peptide transporter PTR2, putative, expressed | | |
| LOC_OS02G20360 | tyrosine aminotransferase, putative, expressed | | DNA binding protein, putative, expressed |
| LOC_OS02G20360 | tyrosine aminotransferase, putative, expressed | | DNA bidning protein, putative, expressed |

Arabidopsis Orthologs of Rice TF

| ID | Description |
|---|---|
| AT1G72070 | TGA3, TGA1A-related gene 3 |
| AT1G77920 | bZIP transcription factor family protein |
| AT5G10030 | OBF4, TGA4, TGACG motif-binding factor 4 |
| AT5G65210 | TGA1, bZIP transcription factor family |
| | protein |
| AT3G50890 | AREB3, DPBF3, ABA-responsive element |
| | binding protein 3 |
| AT1G63030 | ddf2, Integrase type DNA-binding |
| | superfamily protein |
| AT3825710 | ATAIG1, BHLH32, TMO5,basic [symbol] - |
| | loop helix 32 |
| AT1G22070 | TGA3, TGA1A related gene 3 |
| AT1G77920 | |
| AT5G10030 | bZIP transcription factor family protein |
| AT5G55210 | OBF4, TGA4, TGACG motif-binding factor 4 |
| | TGA1, bZIP transcription factor family |
| | protein |
| AT3G26744 | ATICE1, ICE1, SCRM, basic helix- |
| | loop helix (bHLH) DNA binding |
| | superfamily protein |
| AT1645010 | Duplicated homeodomain like superfamily |
| | protein |
| AT3G16350 | Homeodomain-like superfamily protein |

[symbol]

indicates data missing or illegible when filed

### 9. EXAMPLE 4

### 9.1. Building Crop Networks

Network analysis and tools can be used to translate knowledge from models-to-crops to aid in translation to agriculture. By using a publicly available microarray N-treatment dataset of maize that discovered biomarkers nitrogen status in the field, a step-by-step analysis incorporating Arabidopsis network knowledge results in networks that enable focused hypothesis generation with translational value.

5,057 N-responsive genes were identified using functions in VirtualPlant maize, which form a correlation network of 4,278 maize genes. This network is too large to enable focused translational targets, and more than 50% of the maize genes are unannotated. This maize transcriptome data may be interpreted in the context of the Arabidopsis network to derive networks and focused translational targets.

First, the 5,057 maize gnes were mapped to 3,756 arabidopsis homologs using VirtualPlant maize, which uses the maize “best-hit” to Arabidopsis data provided by Phytozyme (www.phytozyme.net).

Next, the “gene network” function in VirtualPlant (protein:protein, metabolic, cis-binding, and text-mining edges) was used to obtain a network of 2,262 connected maize genes. A GO term over-representation test on this network identifies Nitrogen metabolic process (p<1e⁻³³) and sulfur metabolic process (p<0.005) among the significant terms. Hyoptheses were focused for translational studies using conserved N-networks, and the maize translational network was refined by selecting genes that are N-regulated in both maize and Arabidopsis in Step 3.

Subsequently, an Arabidopsis nitrogen response gene set (1,254 genes) was created as a union of genes responsive in shoots (Gutierrez et al., 2008, Proc Natl Acad Sci USA, 105(12):4939) and roots (Schena and Yamamoto, 1988, Science 241(4868):965). These Arabidopsis genes and the 2,262 maize genes were intersected to produce a highly significant (p<0.001) overlapping gene list of 223 N-regulated genes. The regulatory edges in this conserved network were required to have a correlation of >0.7 or <−0.7 (within maize), as described in (Gutierrez et al., 2008, Proc Natl Acad Sci USA, 105(12):4939) and (Sheen, 2001, Plant physiology 149(3):1231). BioMaps analysis in Virtual plant uncovered significant GO terms including photoperiodism (p-val<0.005) and nitrate transport (p-val<0.01) and 15 TF hubs for focused generation of translational targets.

Using the VirtualPlant-meets-Cytoscape function, a “hubbiness” table was generated to identify the master regulatory nodes in the core N-regulatory network conserved between maize and Arabidopsis. Remarkably, the 5 top TF hubs include TFs (CCA1, GLK1 and bZIP9) (FIG. 22) previously validated in Arabidopsis as major regulators of an organic N-response network to regulate genes involved in N-assimilation, including ASN1 (Gutierrez et al., 2008, Proc Natl Acad Sci USA, 105(12):4939; Baena-Gonzalez, 2010, Mol Plant 3(2):300). Components of this network-including AS and a bZIP TF have also been implicated in NUE studies of maize by QTL analysis and Q-PCR.

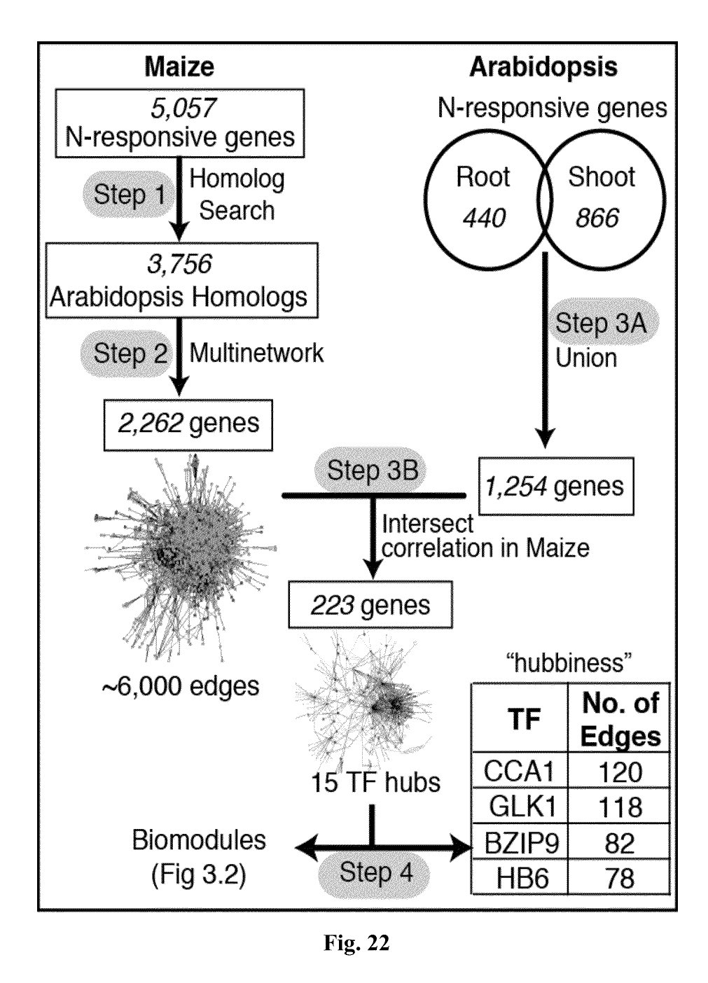

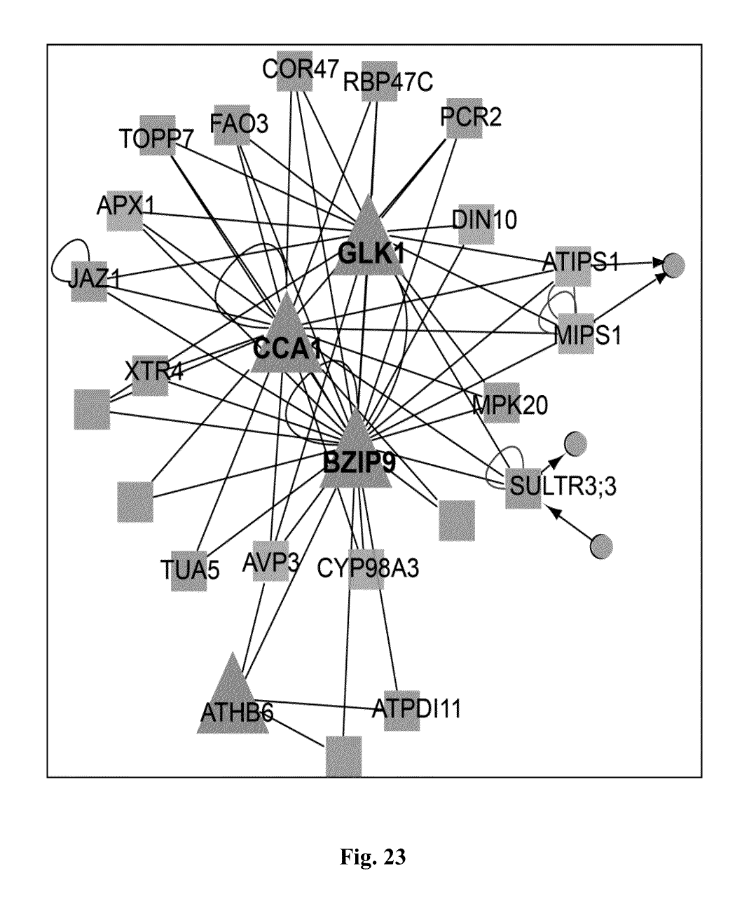

The TF hubs of this N-regulatory network between maize and Arabidopsis (FIG. 22) provide a focus for network module identification and translational targeting. For example, a conserved network module (FIG. 23) shows several TF hubs previously validated to regulate genes involved in N-assimilation in Arabidopsis (Gutierrez et al., 2008, Proc Natl Acad Sci USA, 105(12):4939). Additionally, the likely maize ortholog of Arabidopsis bZIP1 lies within a strong QTL for NUE in maize (Moose lab, unpublished). This netork module also reinforces the discovery that nitrogen-regulation of CCA1 imparts nutrient regulation of N-assimilation and the circadian clock in Arabidopsis (Gutierrez et al., 2008, Proc Natl Acad Sci USA, 105(12):4939) and now in maize. This conserved network also suggests nitrogen influences sulfure uptake (e.g. sulfur transporter gene).

### 10. EXAMPLE 5

### 10.1. Introduction

Signal propagation through gene regulatory networks (GRNs) enables organisms to rapidly respond to changes in environmental signals. For example, dynamic GRN studies in plants have uncovered genome-wide responses that occur within as little as three minutes following a nitrogen (N) nutrient signal perturbation (Kouk et al., 2010, Genome Biology 11:R123). Yet, many of the underlying rapid and temporal network connections between transcription factors (TFs) and their targets elude detection even in fine-scale time-course studies (Ni et al., 2009, Gene Dev 23(11):1351-1363; Chang et al., 2013, Elife 2:e00675), as current methods used (e.g. chromatin immunoprecipitation, ChIP) require stable TF-binding in at least one time-point to identify primary targets (Gorski et al, 2011, Nucleic Acids Research 39(22):9536-9548; Hughes et al., 2013, Genetics 195(1):9-36; Marchive et al., 2013, Nature Communications 4). However, recent models suggest that GRNs built solely on TF-binding data are insufficient to recapture transcriptional regulation (Biggin MD, 2011, Dev Cell 21(4):611-626; Walhout AJM, 2011, Genome Biol 12(4); Lickwar et al., 2012, Nature 484(7393):251-255). Compounding this dilemma, TFs have been found to stably bind to only a small percentage (5-32%) of the TF-regulated genes across eukaryotes (Gorski et al, 2011, Nucleic Acids Research 39(22):9536-9548; Hughes et al., 2013, Genetics 195(1):9-36; Marchive et al., 2013, Nature Communications 4; Monke et al., 2012, Nucleic Acids Research 40:82401; Arenhart et al., 2014, Molecular plant 7(4):709-721; Bolduc et al., 2012, Gene Dev 26(15):1685-1690; Bianco et al., 2014, Cancer research 74(7):2015-2025). Since TF-binding is required to define the primary targets in current GRN studies, the large set of TF-regulated, but not TF-bound genes must be categorically dismissed as indirect or secondary targets (Gorski et al, 2011, Nucleic Acids Research 39(22):9536-9548; Hughes et al., 2013, Genetics 195(1):9-36; Arenhart et al., 2014, Molecular plant 7(4):709-721; Bolduc et al., 2012, Gene Dev 26(15):1685-1690; Bianco et al., 2014, Cancer research 74(7):2015-2025). Provided herein is an alternative—and more intriguing conclusion—that these typically dismissed targets comprise the “dark matter” of rapid and transient signal transduction that has previously eluded detection across eukaryotes.

To capture these rapid and dynamic network connections that elude detection by biochemical TF-binding assays, an approach was developed that can identify primary targets based on a functional read out—TF-induced gene regulation—even in the absence of detectable TF-binding. This study focuses on the master TF bZIP1 (BASIC LEUCINE ZIPPER 1), a central integrator of metabolic signaling including sugar (Baena-Gonzalez et al., 2007, Nature 448:938; Kang et al., 2010, Molecular Plant 3:361-373; Dietrich et al., 2011, The Plant Cell 23:381-395) and N nutrient signals (Gutierrez et al., 2008, Proc. Natl. Acad. Sci. U.S.A. 105:4939; Obertello et al., 2010, BMC Systems Biology 4:111). To uncover the underlying dynamic GRNs, both bZIP1 and the N-signal it transduces were temporally perturbed in a cell-based system designed for temporal TF perturbation. This cell-based system named TARGET (Transient Assay Reporting Genome-wide Effects of Transcription factors), which involves inducible TF nuclear localization, is able to identify primary TF targets based solely on TF-induced gene regulation, as shown for a well-studied TF involved in plant hormone signaling—ABI3 (Bargmann et al., 2013, Molecular Plant 6(3):978). In this study, by adapting a micro-ChIP protocol (Dahl et al., 2008, Nucleic Acids Research, 36:e15) to the cell-based TARGET system, primary targets were monitored based on either TF-induced gene regulation or TF-binding quantified in the same cell samples, enabling a direct comparison. The use of isolated cells allowed the capture of rapid and transient regulatory events including the formation of TF-DNA complexes within 1-5 min from the onset of TF translocation to the nucleus. Such a short-lived interaction would likely be missed in planta, as effective protein-DNA cross-linking in intact plant tissues requires prolonged (for a minimum of 15 minutes) infiltration under vacuum. Unexpectedly, the primary TF targets that are regulated by, but not stably bound to bZIP1—termed “transient”—were the most biologically relevant to rapid transduction of the N-signal. These transient TF-targets include first-responder genes, induced as early as 3-6 minutes after N-signal perturbation in planta (Kouk et al., 2010, Genome Biology 11:R123). This discovery suggests that the current “gold-standard” of GRNs built solely on the intersection of TF-binding and TF-regulation data miss a large and important class of transient TF targets, which are at the heart of dynamic networks. Moreover, the shared features of these transient bZIP1 targets and their role in rapid N-signaling provides genome-wide support for a classic, but largely forgotten model of “hit-and-run” transcription (Schaffner, 1988, Nature 336:427-428). This transient mode-of-action can enable a master TF to catalytically and rapidly activate a large set of genes in response to a signal.

### 10.2. Materials and Methods

Plant Materials and DNA Constructs.

Wild-type Arabidopsis thaliana seeds [Columbia ecotype (Col-0)] were vapor-phase sterilized, vernalized for 3 days, then 1 ml of seed were sown on agar plates containing 2.2 g/l custom made Murashige and Skoog salts without N or sucrose (Sigma-Aldrich), 1% [w/v] sucrose, 0.5 g/l MES hydrate (Sigma-Aldrich), 1 mM KNO3 and 2% [w/v] agar. Plants were grown vertically on plates in an Intellus environment controller (Percival Scientific, Perry, Iowa), whose light regime was set to 50 μmol m⁻² s⁻¹ and 16 h-light/8 h-dark at constant temp of 22° C. The bZIP1 (At5g49450) cDNA in pENTR was obtained from the REGIA collection (Paz-Ares et al., 2002 Comp Funct Genomics 3(2):102-108) and was then cloned into the destination vector pBeaconRFP_GR used in the protoplast expression system (Bargmann et al., 2009, Plant physiology 149:1231) by LR recombination (Life Technologies). The pBeaconRFP_GR vector is available through the VIB website (http://gateway.psb.ugent.be/).

Protoplast Preparation, Transfection, Treatments and Cell Sorting.

Root protoplasts were prepared, transfected and sorted as previously described (Bargmann et al., 2013, Molecular Plant 6(3):978; Yoo et al., 2007, Nature Protocols 2:1565; Bargmann et al., 2009, Plant physiology 149:1231). Briefly, roots of 10-day-old seedlings were harvested and treated with cell wall digesting enzymes (Cellulase and Macerozyme; Yakult, Japan) for 4 h. Cells were filtered and washed then transfected with 40 μg of pBeaconRFP_GR::bZIP1 plasmid DNA per 1×106 cells facilitated by polyethylene glycol treatment (PEG; Fluka 81242) for 25 minutes (Bargmann et al., 2009, Plant physiology 149:1231). Cells were washed drop-wise, concentrated by centrifugation, then resuspended in wash solution W5 (154 mM NaCl, 125 mM CaCl₂, 5 mM KCl, 5 mM MES, 1 mM Glucose) for overnight incubation at room temperature. Protoplast suspensions were treated sequentially with: 1) a N-signal treatment of either a 20 mM KNO3 and 20 mM NH4NO3 solution (N) or 20 mM KCl (control) for 2 h, 2) either CHX (35 μM in DMSO, Sigma-Aldrich) or solvent alone as mock for 20 min, and then 3) with either DEX (10 μM in EtOH, Sigma-Aldrich) or solvent alone as mock for 5 h at room temperature. Treated protoplast suspensions were FACS sorted as in (13): approximately 10,000 RFP-positive cells were FACS sorted directly into RLT buffer (QIAGEN) for RNA extraction.

RNA Extraction and Microarray.

RNA from 6 replicates (3 treatment replicates and 2 biological replicates) was extracted from protoplasts using an RNeasy Micro Kit with RNase-free DNaseI Set (QIAGEN and quantified on a Bioanalyzer RNA Pico Chip (Agilent Technologies). RNA was then converted into cDNA, amplified and labeled with Ovation Pico WTA System V2 (NuGEN) and Encore Biotin Module (NuGEN), respectively. The labeled cDNA was hybridized, washed and stained on an ATH1-121501 Arabidopsis Genome Array (Affymetrix) using a Hybridization Control Kit (Affymetrix), a GeneChip Hybridization, Wash, and Stain Kit (Affymetrix), a GeneChip Fluidics Station 450 and a GeneChip Scanner (Affymetrix).

Analysis of Microarray Data with CHX Treatment.

Microarray intensities were normalized using the GCRMA (http://www.bioconductor.org/packages/2.11/bioc/html/gcrma.html) package. Differentially expressed genes were then determined by a 3-way ANOVA with N, DEX and biological replicates as factors. The raw p-value from ANOVA was adjusted by False Discovery Rate (FDR) to control for multiple testing (Benjamini et al., 2005, Genetics 171:783). Genes significantly regulated by the N-signal and/or DEX-induced bZIP1 nuclear localization were then selected with a FDR cutoff of 5%. Genes significantly regulated by the interaction of the N-signal and bZIP1 (N-signal×bZIP1) were selected with a p-val (ANOVA) cutoff of 0.01. Only unambiguous probes were included. Heat maps were created using Multiple Experiment Viewer software (TIGR; http://www.tm4.org/mev/). The significance of overlaps of gene sets were calculated using the GeneSect (R)script (Katari et al., 2010, Plant physiology 152:500) using the microarray as background. Hypergeometric distribution was used in one case (specified in the manuscript) to evaluate the enrichment of gene sets, when a specific background—N-responsive genes identified in different root cell types (Gifford et al., 2008, Proc. Natl. Acad. Sci. U.S.A. 105:803-808)—was needed.

Filtering bZIP1 targets for the effects of protoplasting, and response to CHX or DEX. In this step, genes were filtered out whose expression states responded to protoplasting, or to treatments of DEX or CHX that were not related to the bZIP1 mediated regulation, in the following three steps: Filter 1: DEX-response filter: Genes responding to DEX independent of TF. Genes significantly induced/repressed by DEX-treatment in protoplasts transfected with the empty pBeanconRFP_GR plasmid (ANOVA analysis; FDR<0.05), were excluded from analysis (1.6% genes filtered). Filter 2: Protoplast-response filter: Genes induced by protoplasting. Genes that are induced by root protoplasting (Birnbaum K, et al., 2003, Science 302(5652):1956-1960) were removed from the list of bZIP1 targets (12.3% genes filtered). Filter 3: DE××CHX interaction filter. Genes whose DEX-regulation is modified by CHX. This filter removes genes from the analysis in cases where the effects of DEX-induced TF nuclear import on gene regulation are affected by CHX treatment. To do this, a 3-way ANOVA was performed (Factors Nitrogen, DEX, and CHX) and bZIP1 primary targets were identified whose gene expression regulation by the DEX-induced nuclear import of bZIP1 is different between +CHX and −CHX conditions (FDR cutoff of interaction term CHX*DEX<0.05). This eliminated genes that are regulated by bZIP1 in the presence of CHX, but not in the absence of CHX. This gene set may contain bZIP1 targets under a self-control negative feedback loop, and bZIP1 targets for which the half-lives of the transcripts affected by CHX. While the first case is potentially interesting, the second case represents the CHX artifact to be removed. Since it is difficult to differentiate between the two outcomes, these CHX-sensitive DEX-responsive genes dependent on bZIP1 were eliminated from the list of bZIP1 target genes (17.4% genes filtered), thus increasing precision over recall.

Micro-Chromatin Immunoprecipitation.

For each combination of protoplast treatments (see above), an unsorted suspension of protoplasts containing approximately 5,000-10,000 GR::bZIP1 transfected cells was fixed for ChIP analysis, using an adapted version of the micro-ChIP protocol by Dahl et al (Dahl et al., 2008, Nucleic Acids Research 36:e15). The advantage in a ChIP analysis from protoplasts is that short-lived interactions would likely be missed in planta assays, as effective protein-DNA cross-linking in intact plant tissues requires prolonged (for a minimum of 15 minutes) infiltration under vacuum (Gendrel et al., 2005, Nat Methods 2(3):213-218). Cells were incubated with gentle rotation in 1% formaldehyde in W5 buffer for 7 minutes, then washed with W5 buffer and frozen in liquid N2. μChIP was performed according to Dahl et al. (2008, Nucleic Acids Research 36:e15) with a few modifications below. The GR::bZIP1-DNA complexes were captured using anti-GR antibody [GR(P-20) (Santa Cruz biotech) bound to Protein-A beads (Life Biotechnologies)]. A washing step with LiCl buffer [0.25M LiCl, 1% Na deoxycholate, 10 mM Tris-HCl (pH8), 1% NP-40] was added in between the wash with RIPA buffer and TE (Dahl et al., 2008, Nucleic Acids Research 36:e15). After elution from the beads, the ChIP material and the Input DNA were cleaned and concentrated using QIAGEN MiniElute Kit (QIAGEN). The protoplast suspension used for micro-ChIP was not FACS sorted in order to maintain a comparable incubation time between the samples that were used for microarray analyses and for micro ChIP. Importantly, while FACS sorting of transformed cells is required for microarray studies, it was not required to identify DNA targets using ChIP-seq.

ChIP-Seq Library Preparation.

The ChIP DNA and Input DNA were prepared for Illumina HiSeq sequencing platform following the Illumina ChIP-Seq protocol (Illumina, San Diego, Calif.) with modifications. Barcoded adaptors and enrichment primers (BiOO Scientific, TX, USA) were used according to the manufacturer's protocol. The concentration and the quality of the libraries was determined by the Qubit Fluorometric DNA Assay (InVitrogen, NY, USA), DNA 12000 Bioanalzyer chip (Agilent, CA, USA) and KAPA Quant Library Kit for Illumina (KAPA Biosystems, MA, USA). A total of 8 libraries were then pooled in equimolar amounts and sequenced on two lanes of an Illumina HiSeq platform for 100 cycles in paired-end configuration (Cold Spring Harbor Lab, NY).

ChIP-Seq Analysis.

Reads obtained from the four treatments (with DEX and N in the presence of CHX) were filtered and aligned to the Arabidopsis thaliana genome (TAIR10) and clonal reads were removed. The ChIP alignment data was compared to its partner Input DNA and peaks were called using the QuEST package (20) with a ChIP seeding enrichment ≧3, and extension and background enrichments ≧2. These regions were overlapped with the genome annotation to identify genes within 500 bp downstream of the peak. The gene lists from multiple treatments were largely overlapping sets, and hence were pooled to generate a single list of genes that show significant binding of bZIP1. Due to technical issues, the experimental design used for ChIP-Seq precludes the observation of significant differences between the genes bound by bZIP1 under the different treatment conditions. This is because the samples fixed for ChIP included a variable number of transfected cells that were not sorted by FACS.

The ChIP-seq studies were performed using a micro-ChIP protocol on ˜10,000 cells, which result in a low DNA input, compared to standard ChIP studies. It has been shown that peak discovery from ChIP data becomes more challenging as the number of cells goes down (FIG. 3 in Gilfillan et al., 2012, Bmc Genomics, 13). Therefore, ChIP libraries made from these very low input-DNA samples have a higher level of background noise, necessitating lower peak calling thresholds. However, even with this caveat for micro-ChIP studies, we were able to recover 850 targets including several previously validated bZIP1 targets (ASN1 and PropH) (Dietrich et al., 2011, The Plant Cell 23:381-395).

Time-Series ChIP-seq.

The ChIP time-series samples were pre-treated with a N-signal treatment of 20 mM KNO3 and 20 mM NH4NO3 solution (N) for 2 h, followed by CHX (35 μM in DMSO, Sigma-Aldrich) for 20 min. Protoplasts were then treated with DEX (10 μM in Ethanol, Sigma-Aldrich) and samples were harvested at 1, 5, 30 and 60 min after the start of the DEX-induced bZIP1 nuclear localization.

Cis-Element Motif Analysis.

1 Kb regions upstream of the TSS (Transcription Start Site) for target genes were extracted based on TAIR10 annotation and submitted to the Elefinder program (all promoters from the genome as background) (Li et al., 2011, Plant physiology 156:2124-2140) or MEME (against a randomized dinucleotide background) (Bailey et al., 2009, Nucleic Acids Research 37:W202-208) to determine over-representation of known cis-element binding sites (different parameters used in specific cases were notified in the paper if applicable). The E-value of significance for each motif was used to cluster the occurrence of motifs in the various subsets using the HCL algorithm in MeV (Saeed et al., 2006, Methods in Enzymology 411:134-193). Motifs that show a higher specificity to a particular category or a sub-group were identified with the PTM algorithm in MeV. De novo motif identification was performed on 1 Kb upstream sequence of the genes regulated by bZIP1 from microarray and ChIP-Seq data separately using the MEME suite (Bailey et al., 2009, Nucleic Acids Research 37:W202-208).

Accession Numbers.

The raw data from all Microarray assays, were submitted to NCBI GEO and is available under the accession number GSE54049. The raw sequencing data from ChIP-Seq assays is available from NCBI SRA under the accession SRX425878.

### 10.3. Results

Temporal Perturbation of Both bZIP1 and the N-Signal it Transduces.

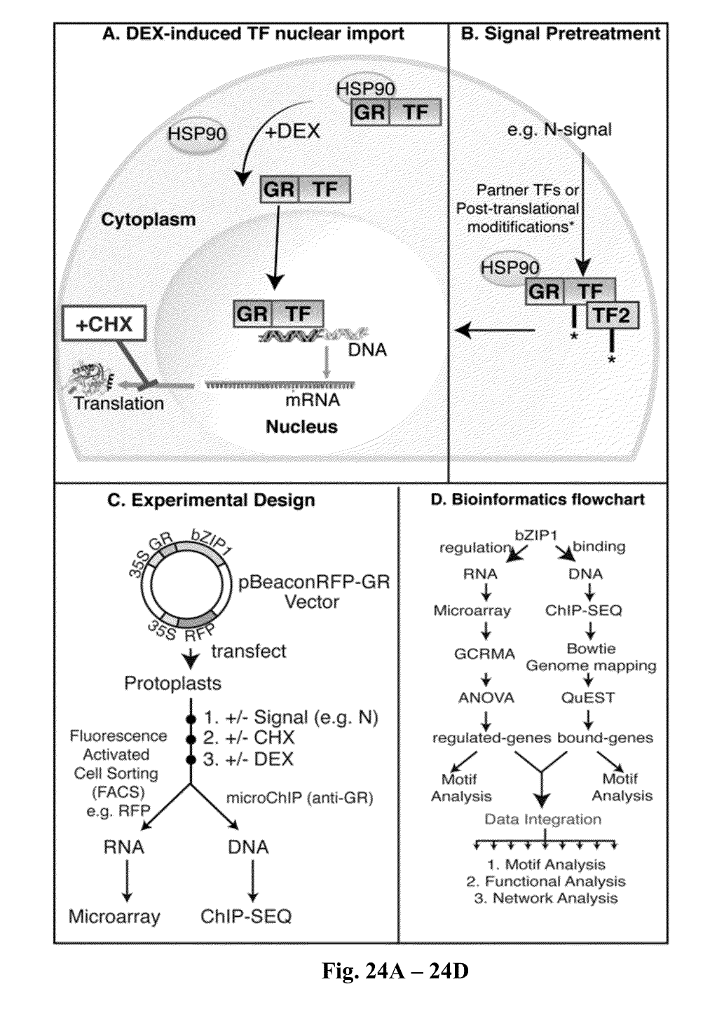

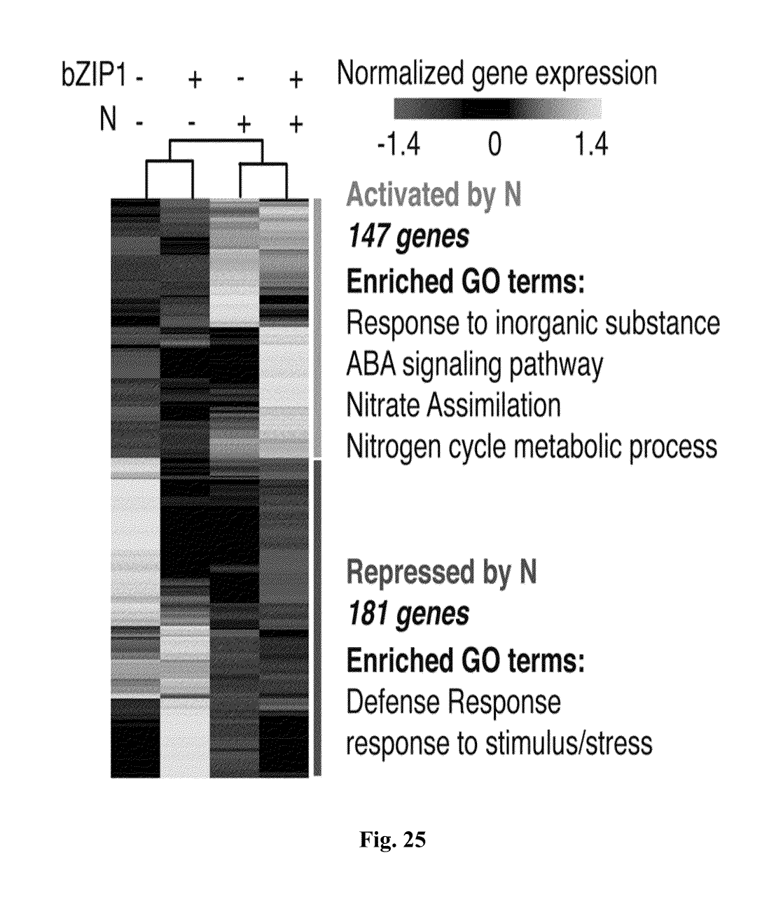

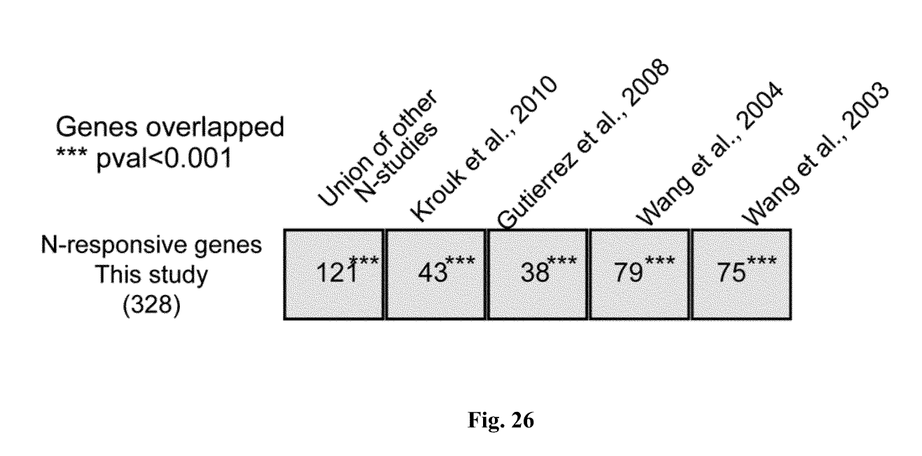

To identify how bZIP1 mediates the rapid propagation of a N-signal in a GRN, both bZIP1 and the N-signal it transduces were temporally perturbed in the cell-based TARGET system (FIGS. 24 A&B) (Bargmann et al., 2013, Molecular Plant 6(3):978). bZIP1, which is ubiquitously expressed across all root cell-types (Birnbaum K, et al., 2003, Science 302(5652):1956-1960), was transiently overexpressed in root protoplasts as a GR::bZIP1 fusion protein, enabling temporal induction of nuclear localization by dexamethasone (DEX) (FIG. 24A) (Bargmann et al., 2013, Molecular Plant 6(3):978). Transfected root cells expressing the GR::bZIP1 fusion protein were sequentially treated with: 1) inorganic nitrogen (+/−N), 2) cycloheximide (+/−CHX) and 3) dexamethasone (+/−DEX) (FIG. 24C). The N-treatment can induce post-translational modifications of bZIP1 (Baena-Gonzalez et al., 2007, Nature 448:938-942), or influence bZIP1 partners by transcriptional or post-transcriptional mechanisms (FIG. 24B). DEX-treatment induces TF nuclear import (FIG. 24A) (Bargmann et al., 2013, Molecular Plant 6(3):978). Further, genes regulated by DEX-induced TF import are deemed primary targets, as a CHX pre-treatment blocks translation of downstream regulators, as previously shown in the TARGET system (Bargmann et al., 2013, Molecular Plant 6(3):978) and in planta (Eklund et al., 2010, Plant Cell 22:349-363) (FIG. 24A). Importantly, to eliminate any side effects caused by CHX pre-treatment, only genes whose transcriptome response to DEX-induced TF nuclear import is the same in either the presence or absence of CHX were considered. Such bZIP1 primary targets identified based on gene regulation following DEX-induced TF import, were identified using Affymetrix ATH1 microarrays. In parallel, primary targets identified by TF-binding were identified in a micro-ChIP-Seq assay (Dahl et al., 2008, Nucleic Acids Research 36:e15) using anti-GR antibodies. Both transcriptome and ChIP-seq data were obtained 5 hours after the DEX-induced nuclear import of bZIP1, from the same cell samples, enabling a direct comparison (FIGS. 24 C&D). Regarding the N-signal, 328 N-responsive genes were identified in the cell-based experiments (FIG. 25; Table 12). These N-responsive genes significantly overlap with the N-responsive genes identified in whole seedlings exposed to a similar N-treatment (NH₄NO₃) (Gutierrez et al., 2008, Proc. Natl. Acad. Sci. U.S.A. 105:4939-4944), and from roots treated with nitrate (Wang et al., 2003, Plant Physiol. 132(2):556-567; Wang et al., 2004, Plant physiology 136(1):2512-2522), including a dynamic study (Krouk et al., 2010, Genome Biology 11:R123) (121/328, p-val<0.001) (FIG. 26; Table 13). The N-responsive genes in the cell-based experiments are enriched with genes that respond to N-treatment across all root cell-types in planta (p-val=8.8E-13, hypergeometric distribution) (Gifford et al., 2008, Proc. Natl. Acad. Sci. U.S.A. 105:803-808).

TABLE 12
N-responsive genes (FDR < 0.05) in root protoplasts used in the TARGET system.

| | | |
|---|---|---|
| Locus | Symbol | FullName |

A. Genes that are up-regulated by N-treatment (FDR < 0.05)

| | | |
|---|---|---|
| AT3G17790 | ATACP5 | |
| AT4G39260 | ATGRP8 | GLYCINE-RICH PROTEIN 8 |
| AT3G20770 | AtEIN3 | |
| AT2G38530 | cdf3 | cell growth defect factor-3 |
| AT3G47420 | AtG3Pp1 | Glycerol-3-phosphate permease 1 |
| AT3G61860 | At-RS31 | arginine/serine-rich splicing factor 31 |
| AT4G13250 | NYC1 | NON-YELLOW COLORING 1 |
| AT4G24620 | PGI | |
| AT5G19430 | | |
| AT4G11560 | | |
| AT5G24870 | | |
| AT1G20110 | | |
| AT2G01850 | ATXTH27 | |
| AT4G14930 | | |
| AT1G19730 | ATH4 | thioredoxin H-type 4 |
| AT3G60750 | | |
| AT5G01340 | AtmSFC1 | |
| AT5G04540 | AtMTM2 | |
| AT3G56150 | ATEIF3C-1 | |
| AT5G48180 | AtNSP5 | |
| AT4G00940 | | |
| AT5G53460 | GLT1 | NADH-dependent glutamate synthase 1 |
| AT1G25550 | | |
| AT4G36760 | APP1 | aminopeptidase P1 |
| AT1G23820 | SPDS1 | spermidine synthase 1 |
| AT3G10740 | ARAF | ALPHA-L-ARABINOFURANOSIDASE |
| AT4G32070 | Phox4 | Phox4 |
| AT2G21290 | | |
| AT5G07890 | | |
| AT3G62140 | | |
| AT3G19030 | | |
| AT5G11470 | | |
| AT4G17340 | DELTA-TIP2 | |
| AT1G04400 | AT-PHH1 | |
| AT3G49620 | DIN11 | DARK INDUCIBLE 11 |
| AT2G26150 | ATHSFA2 | heat shock transcription factor A2 |
| AT3G58610 | | |
| AT1G64190 | | |
| AT1G74310 | ATHSP101 | heat shock protein 11 |
| AT2G26980 | CIPK3 | CBL-interacting protein kinase 3 |
| AT4G12400 | Hop3 | Hop3 |
| AT1G68720 | ATTADA | ARABIDOPSIS THALIANA TRNA ADENOSINE DEAMINASE A |
| AT1G27300 | | |
| AT2G18550 | ATHB21 | homeobox protein 21 |
| AT1G78050 | PGM | phosphoglycerate/bisphosphoglycerate mutase |
| AT3G19290 | ABF4 | ABRE binding factor 4 |
| AT4G27910 | ATX4 | |
| AT2G18050 | HIS 1-3 | histone H1-3 |
| AT5G12860 | DiT1 | dicarboxylate transporter 1 |
| AT5G41670 | | |
| AT3G49630 | | |
| AT4G09620 | | |
| AT1G54050 | | |
| AT2G03270 | | |
| AT5G48570 | ATFKBP65 | |
| AT4G24000 | ATCSLG2 | ARABIDOPSIS THALIANA CELLULOSE SYNTHASE LIKE G2 |
| AT1G65540 | AtLETM2 | |
| AT4G23440 | | |
| AT5G12030 | AT-HSP17.6A | heat shock protein 17.6A |
| AT5G62900 | | |
| AT1G53540 | | |
| AT1G37130 | ATNR2 | ARABIDOPSIS NITRATE REDUCTASE 2 |
| AT3G16050 | A37 | |
| AT1G58360 | AAP1 | amino acid permease 1 |
| AT3G52340 | ATSPP2 | SUCROSE-PHOSPHATASE 2 |
| AT2G16060 | AHB1 | hemoglobin 1 |
| AT5G49470 | | |
| AT1G58080 | ATATP-PRT1 | ATP phosphoribosyl transferase 1 |
| AT1G13300 | HRS1 | HYPERSENSITIVITY TO LOW PI-ELICITED PRIMARY ROOT |
| | | SHORTENING 1 |
| AT5G20790 | | |
| AT5G13180 | ANAC083 | NAC domain containing protein 83 |
| AT5G49000 | | |
| AT5G63680 | | |
| AT1G06570 | HPD | 4-hydroxyphenylpyruvate dioxygenase |
| AT1G55510 | BCDH BETA1 | branched-chain alpha-keto acid decarboxylase E1 beta subunit |
| AT3G52490 | | |
| AT3G60690 | | |
| AT2G38400 | AGT3 | alanine:glyoxylate aminotransferase 3 |
| AT4G23100 | ATECS1 | |
| AT1G09460 | | |
| AT4G38470 | STY46 | serine/threonine/tyrosine kinase 46 |
| AT2G41190 | | |
| AT5G07010 | ATST2A | ARABIDOPSIS THALIANA SULFOTRANSFERASE 2A |
| AT1G23190 | PGM3 | phosphoglucomutase 3 |
| AT5G04630 | CYP77A9 | cytochrome P45, family 77, subfamily A, polypeptide 9 |
| AT3G48360 | ATBT2 | |
| AT4G37540 | LBD39 | LOB domain-containing protein 39 |
| AT1G49500 | | |
| AT1G80160 | GLYI7 | glyoxylase I 7 |
| AT5G47560 | ATSDAT | |
| AT1G53580 | ETHE1 | ETHE1-LIKE |
| AT4G34030 | MCCB | 3-methylcrotonyl-CoA carboxylase |
| AT3G49940 | LBD38 | LOB domain-containing protein 38 |
| AT5G10210 | | |
| AT2G33150 | KAT2 | 3-KETOACYL-COA THIOLASE 2 |
| AT1G03790 | SOM | SOMNUS |
| AT4G31240 | | |
| AT1G04410 | c-NAD-MDH1 | cytosolic-NAD-dependent malate dehydrogenase 1 |
| AT3G13750 | BGAL1 | beta galactosidase 1 |
| AT1G23870 | ATTPS9 | trehalose-phosphatase/synthase 9 |
| AT1G62660 | | |
| AT5G54080 | AtHGO | |
| AT4G09760 | | |
| AT4G38340 | | |
| AT5G52300 | LTI65 | LOW-TEMPERATURE-INDUCED 65 |
| AT1G08190 | ATVAM2 | |
| AT1G14340 | | |
| AT2G45960 | ATHH2 | |
| AT1G23800 | ALDH2B | aldehyde dehydrogenase 2B |
| AT3G01420 | ALPHA-DOX1 | alpha-dioxygenase 1 |
| AT3G16240 | AQP1 | |
| AT5G04250 | | |
| AT4G33080 | | |
| AT2G42560 | | |
| AT5G13110 | G6PD2 | glucose-6-phosphate dehydrogenase 2 |
| AT1G16170 | | |
| AT5G20885 | | |
| AT5G66400 | ATDI8 | ARABIDOPSIS THALIANA DROUGHT-INDUCED 8 |
| AT3G45060 | ATNRT2.6 | ARABIDOPSIS THALIANA HIGH AFFINITY NITRATE TRANSPORTER 2.6 |
| AT2G42750 | | |
| AT3G45300 | ATIVD | |
| AT5G40450 | | |
| AT2G38800 | | |
| AT1G52320 | | |
| AT2G23030 | SNRK2-9 | SUCROSE NONFERMENTING 1-RELATED PROTEIN KINASE 2-9 |
| AT4G35090 | CAT2 | catalase 2 |
| AT3G42860 | | |
| AT3G53540 | TRM19 | TON1 Recruiting Motif 19 |
| AT4G34000 | ABF3 | abscisic acid responsive elements-binding factor 3 |
| AT3G27820 | ATMDAR4 | MONODEHYDROASCORBATE REDUCTASE 4 |
| AT5G48250 | BBX8 | B-box domain protein 8 |
| AT5G50850 | MAB1 | MACCI-BOU |
| AT1G30510 | ATRFNR2 | root FNR 2 |
| AT1G63940 | MDAR6 | monodehydroascorbate reductase 6 |
| AT3G26100 | | |
| AT5G65210 | TGA1 | TGACG sequence-specific binding protein 1 |
| AT1G73920 | | |
| AT1G60710 | ATB2 | |
| AT5G15450 | APG6 | ALBINO AND PALE GREEN 6 |
| AT3G48990 | AAE3 | ACYL-ACTIVATING ENZYME 3 |
| AT2G15620 | ATHNIR | ARABIDOPSIS THALIANA NITRITE REDUCTASE |
| AT5G39590 | | |
| AT1G68670 | | |
| AT5G65660 | | |
| AT3G61430 | ATPIP1 | ARABIDOPSIS THALIANA PLASMA MEMBRANE INTRINSIC PROTEIN 1 |
| AT4G12340 | | |
| AT5G67420 | ASL39 | ASYMMETRIC LEAVES2-LIKE 39 |

B. genes that are down-regulated by N-treatment (FDR < 0.05)

| | | |
|---|---|---|
| AT1G56060 | | |
| AT1G53430 | | |
| AT3G21230 | 4CL5 | 4-coumarate:CoA ligase 5 |
| AT4G02330 | AtPME41 | |
| AT4G01850 | AtSAM2 | |
| AT1G52200 | | |
| AT2G23270 | | |
| AT5G59480 | | |
| AT2G17220 | Kin3 | kinase 3 |
| AT3G10640 | VPS60.1 | |
| AT5G58120 | | |
| AT5G61210 | ATSNAP33 | |
| AT1G10160 | | |
| AT3G15520 | | |
| AT4G19960 | ATKUP9 | |
| AT4G28940 | | |
| AT4G30560 | ATCNGC9 | cyclic nucleotide gated channel 9 |
| AT2G38120 | AtAUX1 | |
| AT3G59900 | ARGOS | AUXIN-REGULATED GENE INVOLVED IN ORGAN SIZE |
| AT4G28850 | ATXTH26 | |
| AT4G39720 | | |
| AT1G09920 | | |
| AT4G24580 | REN1 | ROP1 ENHANCER 1 |
| AT4G39940 | AKN2 | APS-kinase 2 |
| AT1G54690 | G-H2AX | GAMMA H2AX |
| AT3G10940 | LSF2 | LIKE SEX4 2 |
| AT5G01490 | ATCAX4 | |
| AT1G73530 | | |
| AT4G24350 | | |
| AT3G55630 | ATDFD | DHFS-FPGS homolog D |
| AT5G43520 | | |
| AT1G74870 | | |
| AT2G35990 | LOG2 | LONELY GUY 2 |
| AT1G32350 | AOX1D | alternative oxidase 1D |
| AT3G56400 | ATWRKY70 | ARABIDOPSIS THALIANA WRKY DNA-BINDING PROTEIN 7 |
| AT2G47140 | AtSDR5 | |
| AT4G26470 | | |
| AT1G73066 | | |
| AT2G43000 | ANAC042 | NAC domain containing protein 42 |
| AT5G06720 | ATPA2 | peroxidase 2 |
| AT1G09930 | ATOPT2 | oligopeptide transporter 2 |
| AT1G09520 | | |
| AT4G25030 | | |
| AT1G18860 | ATWRKY61 | WRKY DNA-BINDING PROTEIN 61 |
| AT2G39530 | | |
| AT3G02850 | SKOR | STELAR K+ outward rectifier |
| AT5G24540 | BGLU31 | beta glucosidase 31 |
| AT5G39680 | EMB2744 | EMBRYO DEFECTIVE 2744 |
| AT1G16380 | ATCHX1 | |
| AT4G11170 | | |
| AT3G07390 | AIR12 | Auxin-Induced in Root cultures 12 |
| AT5G44060 | | |
| AT1G35200 | | |
| AT1G72070 | | |
| AT2G25735 | | |
| AT2G32020 | | |
| AT3G10630 | | |
| AT1G53920 | GLIP5 | GDSL-motif lipase 5 |
| AT1G18570 | AtMYB51 | myb domain protein 51 |
| AT2G19570 | AT-CDA1 | |
| AT3G08750 | | |
| AT1G30370 | DLAH | DAD1-like acylhydrolase |
| AT3G08730 | ATPK1 | ARABIDOPSIS THALIANA PROTEIN-SERINE KINASE 1 |
| AT1G14540 | PER4 | peroxidase 4 |
| AT5G15130 | ATWRKY72 | ARABIDOPSIS THALIANA WRKY DNA-BINDING PROTEIN 72 |
| AT1G14550 | | |
| AT4G22720 | | |
| AT5G60250 | | |
| AT1G73510 | | |
| AT4G14368 | | |
| AT2G33710 | | |
| AT4G37900 | | |
| AT1G33590 | | |
| AT4G08770 | Prx37 | peroxidase 37 |
| AT3G50790 | | |
| AT4G23570 | SGT1A | |
| AT1G18390 | | |
| AT5G26920 | CBP60G | Cam-binding protein 6-like G |
| AT1G05575 | | |
| AT3G01500 | ATBCA1 | BETA CARBONIC ANHYDRASE 1 |
| AT1G68765 | IDA | INFLORESCENCE DEFICIENT IN ABSCISSION |
| AT5G64650 | | |
| AT3G55090 | ABCG16 | ATP-binding cassette G16 |
| AT4G17785 | MYB39 | myb domain protein 39 |
| AT1G02900 | ATRALF1 | RAPID ALKALINIZATION FACTOR 1 |
| AT3G57080 | NRPE5 | |
| AT5G05220 | | |
| AT3G22900 | NRPD7 | |
| AT1G03990 | | |
| AT4G04490 | CRK36 | cysteine-rich RLK (RECEPTOR-like protein kinase) 36 |
| AT5G14740 | BETA CA2 | BETA CARBONIC ANHYDRASE 2 |
| AT1G76550 | | |
| AT2G29330 | TRI | tropinone reductase |
| AT5G45280 | | |
| AT5G64860 | DPE1 | disproportionating enzyme |
| AT1G54890 | | |
| AT4G18950 | | |
| AT1G02360 | | |
| AT1G10330 | | |
| AT1G76570 | | |
| AT2G44790 | UCC2 | uclacyanin 2 |
| AT2G22870 | EMB2001 | embryo defective 21 |
| AT2G42880 | ATMPK20 | MAP kinase 2 |
| AT1G51680 | 4CL.1 | 4-COUMARATE:COA LIGASE 1 |
| AT1G75960 | | |
| AT1G05670 | | |
| AT2G18190 | | |
| AT1G80240 | DGR1 | DUF642 L-GalL responsive gene 1 |
| AT5G11910 | | |
| AT5G16770 | AtMYB9 | myb domain protein 9 |
| AT1G17300 | | |
| AT5G40770 | ATPHB3 | prohibitin 3 |
| AT1G22890 | | |
| AT5G65930 | KCBP | KINESIN-LIKE CALMODULIN-BINDING PROTEIN |
| AT1G72280 | AERO1 | endoplasmic reticulum oxidoreductins 1 |
| AT5G03620 | | |
| AT2G18180 | | |
| AT1G71400 | AtRLP12 | receptor like protein 12 |
| AT3G29250 | AtSDR4 | |
| AT3G63220 | | |
| AT1G80850 | | |
| AT5G22270 | | |
| AT4G17486 | | |
| AT2G33820 | ATMBAC1 | |
| AT4G23690 | AtDIR6 | Arabidopsis thaliana dirigent protein 6 |
| AT4G09650 | ATPD | ATP synthase delta-subunit gene |
| AT1G03920 | | |
| AT2G43610 | | |
| AT3G22800 | | |
| AT1G13210 | ACA.1 | autoinhibited Ca2+/ATPase II |
| AT1G30750 | | |
| AT1G50590 | | |
| AT5G63040 | | |
| AT5G07110 | PRA1.B6 | prenylated RAB acceptor 1.B6 |
| AT5G63780 | SHA1 | shoot apical meristem arrest 1 |
| AT5G66390 | | |
| AT3G01280 | ATVDAC1 | ARABIDOPSIS THALIANA VOLTAGE DEPENDENT ANION CHANNEL 1 |
| AT2G34610 | | |
| AT2G44380 | | |
| AT3G55150 | ATEXO70H1 | exocyst subunit exo7 family protein H1 |
| AT3G49130 | | |
| AT5G41610 | ATCHX18 | ARABIDOPSIS THALIANA CATION/H+ EXCHANGER 18 |
| AT1G10090 | | |
| AT1G64160 | AtDIR5 | |
| AT3G48650 | | |
| AT5G61440 | ACHT5 | atypical CYS HIS rich thioredoxin 5 |
| AT4G37240 | | |
| AT5G64100 | | |
| AT3G46280 | | |
| AT5G24030 | SLAH3 | SLAC1 homologue 3 |
| AT1G13280 | AOC4 | allene oxide cyclase 4 |
| AT2G10640 | | |
| AT1G02450 | NIMIN-1 | |
| AT3G22920 | | |
| AT1G65840 | ATPAO4 | polyamine oxidase 4 |
| AT3G30396 | | |
| AT3G05210 | ERCC1 | |
| AT5G58630 | TRM31 | TON1 Recruiting Motif 31 |
| AT2G44370 | | |
| AT4G20870 | ATFAH2 | ARABIDOPSIS FATTY ACID HYDROXYLASE 2 |
| AT5G02780 | GSTL1 | glutathione transferase lambda 1 |
| AT1G16150 | WAKL4 | wall associated kinase-like 4 |
| AT3G01175 | | |
| AT5G64120 | | |
| AT2G31380 | BBX25 | B-box domain protein 25 |
| AT4G33420 | | |
| AT1G56150 | | |
| AT2G43620 | | |
| AT1G32930 | | |
| AT3G23230 | AtERF98 | |
| AT3G22890 | APS1 | ATP sulfurylase 1 |
| AT1G68850 | | |
| AT3G23240 | ATERF1 | ETHYLENE RESPONSE FACTOR 1 |
| AT1G71530 | | |
| AT4G26690 | GDPDL3 | Glycerophosphodiester phosphodiesterase (GDPD) like 3 |
| AT5G17990 | pat1 | PHOSPHORIBOSYLANTHRANILATE TRANSFERASE 1 |
| AT2G04500 | | |
| AT5G14470 | | |
| AT2G02180 | TOM3 | tobamovirus multiplication protein 3 |
| AT5G48430 | | |
| AT5G67450 | AZF1 | zinc-finger protein 1 |

TABLE 13
Overlap of N-responsive genes in protoplasts vs. N-response studies performed in planta

| | |
|---|---|
| At4g24620 | PGI, PGI1, phosphoglucose isomerase 1 |
| At3g49940 | LBD38, LOB domain-containing protein 38 |
| At1g52200 | PLAC8 family protein |
| At3g61430 | ATPIP1, PIP1, PIP1;1, PIP1A, plasma membrane intrinsic protein 1A |
| At3g58610 | ketol-acid reductoisomerase |
| At3g21230 | 4CL5, 4-coumarate:CoA ligase 5 |
| At1g73920 | alpha/beta-Hydrolases superfamily protein |
| At5g15130 | ATWRKY72, WRKY72, WRKY DNA-binding protein 72 |
| At5g48180 | NSP5, nitrile specifier protein 5 |
| At4g35090 | CAT2, catalase 2 |
| At5g39590 | TLD-domain containing nucleolar protein |
| At1g23870 | ATTPS9, TPS9, TPS9, trehalose-phosphatase/synthase 9 |
| At4g09620 | Mitochondrial transcription termination factor family protein |
| At2g19570 | AT-CDA1, CDA1, DESZ, cytidine deaminase 1 |
| At5g43520 | Cysteine/Histidine-rich C1 domain family protein |
| At1g05575 | unknown protein; FUNCTIONS IN: molecular_function unknown; INVOLVED IN: anaerobic |
| | respiration; LOCATED IN: endomembrane system; EXPRESSED IN: 17 plant structures; |
| | EXPRESSED DURING: 9 growth stages; BEST Arabidopsis thaliana protein match is: unknown |
| | protein (TAIR:AT2G31945.1); Has 63 Blast hits to 63 proteins in 10 species: Archae - 0; Bacteria - 0; |
| | Metazoa - 0; Fungi - 0; Plants - 63; Viruses - 0; Other Eukaryotes - 0 (source: NCBI BLink). |
| At1g37130 | ATNR2, B29, CHL3, NIA2, NIA2-1, NR, NR2, nitrate reductase 2 |
| At5g22270 | unknown protein; BEST Arabidopsis thaliana protein match is: unknown protein |
| | (TAIR:AT3G11600.1); Has 136 Blast hits to 136 proteins in 15 species: Archae - 0; Bacteria - 0; |
| | Metazoa - 0; Fungi - 0; Plants - 136; Viruses - 0; Other Eukaryotes - 0 (source: NCBI BLink). |
| At5g04540 | Myotubularin-like phosphatases II superfamily |
| At1g56150 | SAUR-like auxin-responsive protein family |
| At5g67420 | ASL39, LBD37, LOB domain-containing protein 37 |
| At5g64100 | Peroxidase superfamily protein |
| At3g19030 | unknown protein; FUNCTIONS IN: molecular_function unknown; INVOLVED IN: pyridoxine |
| | biosynthetic process, homoserine biosynthetic process; LOCATED IN: endomembrane system; |
| | EXPRESSED IN: 19 plant structures; EXPRESSED DURING: 9 growth stages; BEST Arabidopsis |
| | thaliana protein match is: unknown protein (TAIR:AT1G49500.1); Has 22 Blast hits to 22 proteins in 2 |
| | species: Archae - 0; Bacteria - 0; Metazoa - 0; Fungi - 0; Plants - 22; Viruses - 0; Other Eukaryotes - 0 |
| | (source: NCBI BLink). |
| At5g20885 | RING/U-box superfamily protein |
| At1g06570 | HPD, PDS1, phytoene desaturation 1 |
| At5g24870 | RING/U-box superfamily protein |
| At5g04250 | Cysteine proteinases superfamily protein |
| At2g01850 | ATXTH27, EXGT-A3, XTH27, endoxyloglucan transferase A3 |
| At3g07390 | AIR12, auxin-responsive family protein |
| At1g02900 | ATRALF1, RALF1, RALFL1, rapid alkalinization factor 1 |
| At5g01340 | Mitochondrial substrate carrier family protein |
| At1g60710 | ATB2, NAD(P)-linked oxidoreductase superfamily protein |
| At4g00940 | Dof-type zinc finger DNA-binding family protein |
| At2g02180 | TOM3, tobamovirus multiplication protein 3 |
| At1g68720 | ATTADA, TADA, tRNA arginine adenosine deaminase |
| At4g39940 | AKN2, APK2, APS-kinase 2 |
| At3g48360 | ATBT2, BT2, BTB and TAZ domain protein 2 |
| At3g47420 | ATPS3, PS3, phosphate starvation-induced gene 3 |
| At5g12860 | DiT1, dicarboxylate transporter 1 |
| At5g10210 | CONTAINS InterPro DOMAIN/s: C2 calcium-dependent membrane targeting (InterPro:IPR000008); |
| | BEST Arabidopsis thaliana protein match is: unknown protein (TAIR:AT5G65030.1); Has 1807 Blast |
| | hits to 1807 proteins in 277 species: Archae - 0; Bacteria - 0; Metazoa - 736; Fungi - 347; Plants - 385; |
| | Viruses - 0; Other Eukaryotes - 339 (source: NCBI BLink). |
| At4g19960 | ATKUP9, HAK9, KT9, KUP9, K+ uptake permease 9 |
| At1g13280 | AOC4, allene oxide cyclase 4 |
| At3g60750 | Transketolase |
| At2g15620 | ATHNIR, NIR, NIR1, nitrite reductase 1 |
| At1g65840 | ATPAO4, PAO4, polyamine oxidase 4 |
| At5g24030 | SLAH3, SLAC1 homologue 3 |
| At2g16060 | AHB1, ARATH GLB1, ATGLB1, GLB1, HB1, NSHB1, hemoglobin 1 |
| At3g55150 | ATEXO70H1, EXO70H1, exocyst subunit exo70 family protein H1 |
| At2g23030 | SNRK2-9, SNRK2.9, SNF1-related protein kinase 2.9 |
| At1g58360 | AAP1, NAT2, amino acid permease 1 |
| At4g38340 | Plant regulator RWP-RK family protein |
| At2g32020 | Acyl-CoA N-acyltransferases (NAT) superfamily protein |
| At5g48570 | ATFKBP65, FKBP65, ROF2, FKBP-type peptidyl-prolyl cis-trans isomerase family protein |
| At1g62660 | Glycosyl hydrolases family 32 protein |
| At2g34610 | unknown protein; BEST Arabidopsis thaliana protein match is: unknown protein |
| | (TAIR:AT1G30190.1); Has 342 Blast hits to 279 proteins in 74 species: Archae - 0; Bacteria - 7; |
| | Metazoa - 76; Fungi - 18; Plants - 51; Viruses - 0; Other Eukaryotes - 190 (source: NCBI BLink). |
| At1g49500 | unknown protein; FUNCTIONS IN: molecular function unknown; INVOLVED IN: |
| | biological_process unknown; LOCATED IN: endomembrane system; EXPRESSED IN: 19 plant |
| | structures; EXPRESSED DURING: 10 growth stages; BEST Arabidopsis thaliana protein match is: |
| | unknown protein (TAIR:AT3G19030.1); Has 24 Blast hits to 24 proteins in 2 species: Archae - 0; |
| | Bacteria - 0; Metazoa - 0; Fungi - 0; Plants - 24; Viruses - 0; Other Eukaryotes - 0 (source: NCBI |
| | BLink). |
| At1g54690 | G-H2AX, GAMMA-H2AX, H2AXB, HTA3, gamma histone variant H2AX |
| At2g33710 | Integrase-type DNA-binding superfamily protein |
| At3g22890 | APS1, ATP sulfurylase 1 |
| At3g23240 | ATERF1, ERF1, ethylene response factor 1 |
| At1g54050 | HSP20-like chaperones superfamily protein |
| At4g37540 | LBD39, LOB domain-containing protein 39 |
| At1g58080 | ATATP-PRT1, ATP-PRT1, HISN1A, ATP phosphoribosyl transferase 1 |
| At5g50850 | MAB1, Transketolase family protein |
| At5g12030 | AT-HSP17.6A, HSP17.6, HSP17.6A, heat shock protein 17.6A |
| At1g13300 | HRS1, myb-like transcription factor family protein |
| At1g14340 | RNA-binding (RRM/RBD/RNP motifs) family protein |
| At3g60690 | SAUR-like auxin-responsive protein family |
| At2g43620 | Chitinase family protein |
| At5g63780 | SHA1, RING/FYVE/PHD zinc finger superfamily protein |
| At5g59480 | Haloacid dehalogenase-like hydrolase (HAD) superfamily protein |
| At1g09460 | Carbohydrate-binding X8 domain superfamily protein |
| At5g13180 | ANAC083, NAC083, VNI2, NAC domain containing protein 83 |
| At5g62900 | unknown protein; FUNCTIONS IN: molecular_function unknown; INVOLVED IN: N-terminal protein |
| | myristoylation; LOCATED IN: cellular_component unknown; EXPRESSED IN: 22 plant structures; |
| | EXPRESSED DURING: 12 growth stages; BEST Arabidopsis thaliana protein match is: unknown |
| | protein (TAIR:AT5G50090.1); Has 157 Blast hits to 157 proteins in 14 species: Archae - 0; Bacteria - |
| | 0; Metazoa - 0; Fungi - 0; Plants - 157; Viruses - 0; Other Eukaryotes - 0 (source: NCBI BLink). |
| At4g34000 | ABF3, DPBF5, abscisic acid responsive elements -binding factor 3 |
| At2g39530 | Uncharacterised protein family (UPF0497) |
| At2g17220 | Protein kinase superfamily protein |
| At1g64190 | 6-phosphogluconate dehydrogenase family protein |
| At1g14540 | Peroxidase superfamily protein |
| At1g33590 | Leucine-rich repeat (LRR) family protein |
| At1g78050 | PGM, phosphoglycerate/bisphosphoglycerate mutase |
| At1g63940 | MDAR6, monodehydroascorbate reductase 6 |
| At3g59900 | ARGOS, auxin-regulated gene involved in organ size |
| At4g37900 | Protein of unknown function (duplicated DUF1399) |
| At2g26980 | CIPK3, SnRK3.17, CBL-interacting protein kinase 3 |
| At1g50590 | RmlC-like cupins superfamily protein |
| At5g26920 | CBP60G, Cam-binding protein 60-like G |
| At4g34030 | MCCB, 3-methylcrotonyl-CoA carboxylase |
| At5g64120 | Peroxidase superfamily protein |
| At5g65210 | TGA1, bZIP transcription factor family protein |
| At1g18390 | Protein kinase superfamily protein |
| At1g14550 | Peroxidase superfamily protein |
| At5g13110 | G6PD2, glucose-6-phosphate dehydrogenase 2 |
| At2g42880 | ATMPK20, MPK20, MAP kinase 20 |
| At3g10740 | ARAF, ARAF1, ASD1, ATASD1, alpha-L-arabinofuranosidase 1 |
| At2g44380 | Cysteine/Histidine-rich C1 domain family protein |
| At5g53460 | GLT1, NADH-dependent glutamate synthase 1 |
| At5g16770 | AtMYB9, MYB9, myb domain protein 9 |
| At1g23190 | Phosphoglucomutase/phosphomannomutase family protein |
| At3g48990 | AMP-dependent synthetase and ligase family protein |
| At5g47560 | ATSDAT, ATTDT, TDT, tonoplast dicarboxylate transporter |
| At1g76550 | Phosphofructokinase family protein |
| At5g07010 | ATST2A, ST2A, sulfotransferase 2A |
| At1g30510 | ATRFNR2, RFNR2, root FNR 2 |
| At1g30370 | alpha/beta-Hydrolases superfamily protein |
| At1g68670 | myb-like transcription factor family protein |
| At5g45280 | Pectinacetylesterase family protein |
| At4g38470 | ACT-like protein tyrosine kinase family protein |
| At1g16170 | unknown protein; FUNCTIONS IN: molecular_function unknown; INVOLVED IN: |
| | biological_process unknown; LOCATED IN: cellular_component unknown; EXPRESSED IN: 24 |
| | plant structures; EXPRESSED DURING: 15 growth stages; BEST Arabidopsis thaliana protein match |
| | is: unknown protein (TAIR:AT1G79660.1); Has 55 Blast hits to 55 proteins in 13 species: Archae - 0; |
| | Bacteria - 0; Metazoa - 0; Fungi - 0; Plants - 55; Viruses - 0; Other Eukaryotes - 0 (source: NCBI |
| | BLink). |
| At5g41670 | 6-phosphogluconate dehydrogenase family protein |
| At2g43000 | anac042, NAC042, NAC domain containing protein 42 |
| At4g39720 | VQ motif-containing protein |
| At1g51680 | 4CL.1, 4CL1, AT4CL1, 4-coumarate:CoA ligase 1 |
| At3g55090 | ABC-2 type transporter family protein |
| At5g15450 | APG6, CLPB-P, CLPB3, casein lytic proteinase B3 |
| At1g53920 | GLIP5, GDSL-motif lipase 5 |
| At5g07890 | myosin heavy chain-related |
| At3g29250 | NAD(P)-binding Rossmann-fold superfamily protein |
| At1g25550 | myb-like transcription factor family protein |
| At5g48430 | Eukaryotic aspartyl protease family protein |
| At4g37240 | unknown protein; FUNCTIONS IN: molecular_function unknown; INVOLVED IN: N-terminal protein |
| | myristoylation; LOCATED IN: cellular_component unknown; EXPRESSED IN: 22 plant structures; |
| | EXPRESSED DURING: 13 growth stages; BEST Arabidopsis thaliana protein match is: unknown |
| | protein (TAIR:AT2G23690.1); Has 30201 Blast hits to 17322 proteins in 780 species: Archae - 12; |
| | Bacteria - 1396; Metazoa - 17338; Fungi - 3422; Plants - 5037; Viruses - 0; Other Eukaryotes - 2996 |
| | (source: NCBI BLink). |

Primary Targets of bZIP1 can be Identified by Either TF-Regulation or TF-Binding.

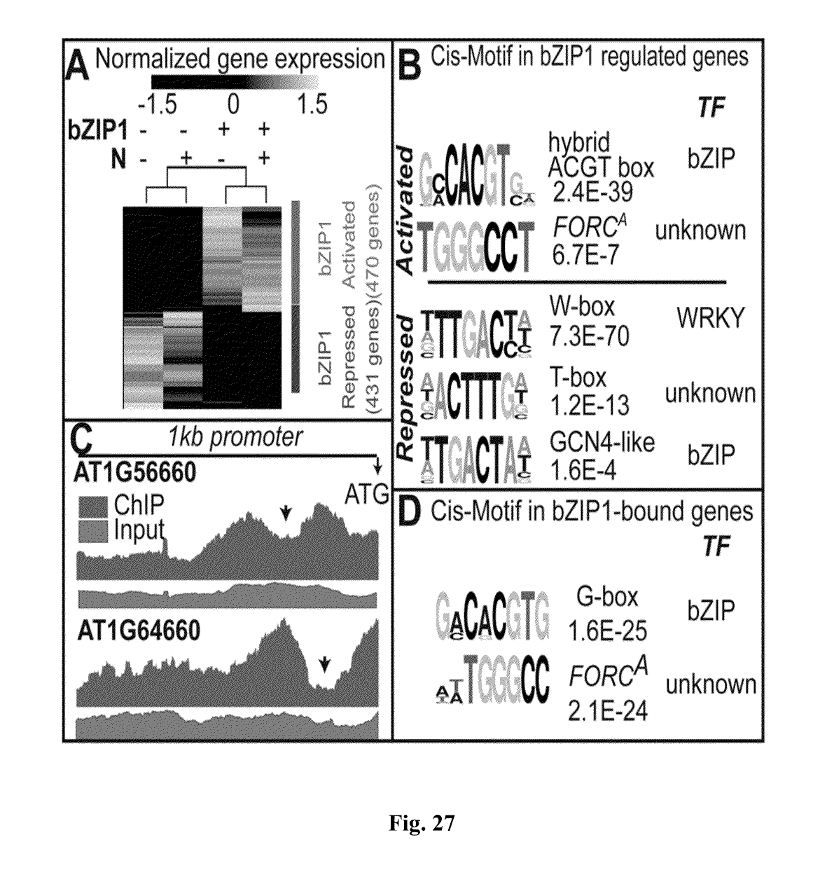

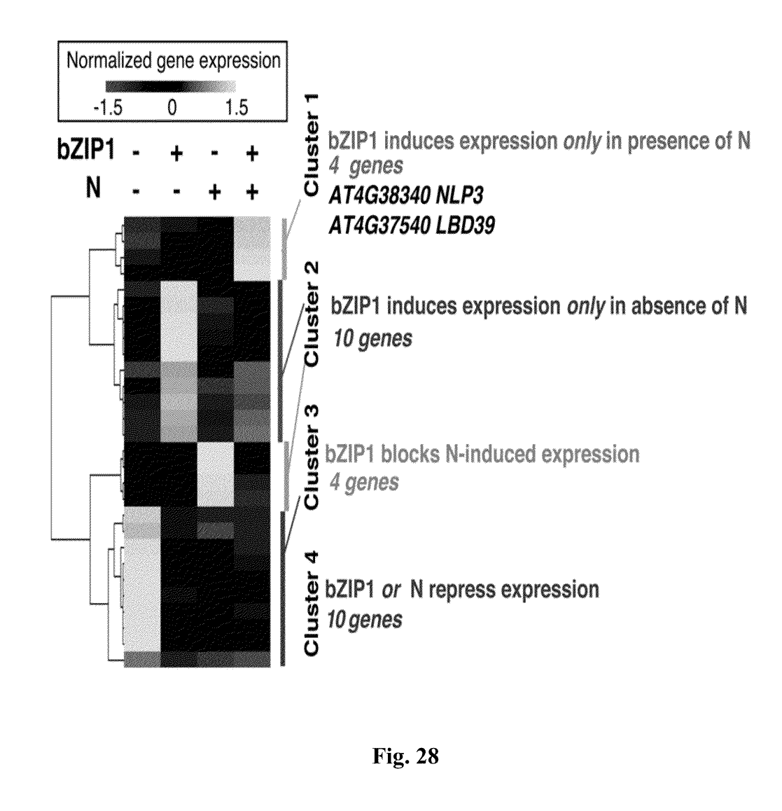

bZIP1 primary targets were first identified based solely on TF-induced gene regulation. A total of 901 genes were identified as primary bZIP1 targets based on significant regulation in response to DEX-induced TF nuclear import, compared to minus DEX controls (ANOVA analysis; FDR adjusted p-value <0.05) (FIG. 27A; FIG. 24D; Tables 14-16). These DEX-responsive genes are deemed to be primary targets of bZIP1, as pre-treatment of the samples with CHX (prior to DEX-induced TF nuclear import) blocks translation of mRNAs of primary bZIP1 targets, thus preventing changes in the mRNA levels of secondary targets in the GRN. To control for the potential side effects of CHX, this list of bZIP1 primary targets excluded genes whose DEX-induced mRNA response was altered by CHX treatment. With regard to the N-signal, 28 out of the 901 bZIP1 primary targets were regulated in response to a significant N-treatment×TF interaction (p-val<0.01) (FIG. 28; Table 17). This could reflect a post-translational modification of bZIP1 by the N-signal, or the N-induced modification of bZIP1 partners at the transcriptional and/or post-translational level (FIG. 24B).

bZIP1 primary targets were next identified based solely on TF-DNA binding. Genes bound by bZIP1 were identified as genic regions enriched in the ChIP DNA, compared to the background (input DNA), using the QuEST peak-calling algorithm (FIG. 27C) (Valouev et al., 2008, Nature Methods 5:829-834). This identified 850 genes with significant bZIP1 binding (FDR<0.05) (FIG. 24D; Table 18), which included validated bZIP1 targets identified by single gene studies (e.g. ASN1 and PropH) (Dietrich et al., 2011, The Plant Cell 23:381-395). It is noted that ChIP-seq can potentially detect genes directly bound to bZIP1, as well as genes indirectly bound by bZIP1 through bridging interactors. Thus, to independently assess whether primary targets identified either by TF-binding or TF-regulation were due to direct binding of bZIP1, cis-element analysis was performed (FIGS. 27 B&D). The bZIP1-bound genes and the bZIP1 regulated genes, are each highly significantly enriched in known bZIP1 binding sites, based on analysis of de novo cis-motifs using MEME (Bailey et al., 2009, Nucleic Acids Research 37:W202-208) or known cis-motif enrichment using Elefinder (Li et al., 2011, Plant physiology 156:2124-2140) (FIGS. 27 B&D).

TABLE 14
Genes identified to be ZIP1 targets based on ANOVA analysis
of transcriptome and/or by ChIP-Seq analysis.

| | |
|---|---|
| Category of Genes | Number of Genes |

Microarray Analysis

| | | |
|---|---|---|
| Significantly regulated | Nitrogen (FDR < 0.05) | 328 |
| by ANOVA factor | bZIP1 (FDR < 0.05) | 901 |
| | NitrogenXbZIP1 (pval < 0.01) | 82 |
| | bZIP1 (FDR < 0.05) AND | 28 |
| | NitrogenXbZIP1 (pval < 0.01) | |

ChIP-SEQ Analysis

| | |
|---|---|
| bZIP1 bound genes | 850 |

In italic: genes considered as TF primary targets in this study.

TABLE 15
bZIP1 primary targets identified as genes up-regulated or
down-regulated by DEX-induced nuclear import of bZIP1 (FDR < 0.05).

| | mean | mean | mean | mean |
|---|---|---|---|---|
| | expression | expression | expression | expression |
| | level | level | level | level |
| | (−N/−Dex) | (−N/+Dex) | (+N/−Dex) | (+N/+Dex) |
| A. Genes | | | | |
| that are | | | | |
| up-regulated | | | | |
| by DEX | | | | |
| (FDR < 0.05) | | | | |
| AT3G01290 | 10184.42 | 11470.63 | 9717.07 | 11446.96 |
| AT5G07440 | 7205.35 | 10345.22 | 7677.83 | 10608.98 |
| AT1G73260 | 8932.55 | 9699.11 | 9311.45 | 10476.40 |
| AT5G52050 | 8413.93 | 9799.60 | 8527.51 | 10023.89 |
| AT3G30775 | 5957.48 | 9395.19 | 5816.87 | 9054.40 |
| AT5G01600 | 4784.75 | 7836.06 | 5315.54 | 7812.26 |
| AT3G60140 | 4631.95 | 7436.68 | 5268.33 | 7486.52 |
| AT5G40780 | 6351.57 | 7390.86 | 6651.01 | 7327.18 |
| AT5G12340 | 6655.56 | 7648.56 | 6683.99 | 7311.89 |
| AT1G69490 | 4711.22 | 7387.64 | 4805.96 | 7198.73 |
| AT3G45970 | 4244.45 | 6891.99 | 4430.99 | 6987.22 |
| AT5G03380 | 5921.87 | 6920.93 | 6001.52 | 6778.08 |
| AT5G28050 | 2746.82 | 7454.05 | 2832.16 | 6293.82 |
| AT2G34600 | 5129.64 | 6443.44 | 5192.91 | 6111.11 |
| AT5G64120 | 5699.56 | 7021.79 | 5052.95 | 6031.23 |
| AT4G15610 | 5307.80 | 6201.30 | 5323.19 | 5859.60 |
| AT1G80380 | 3193.36 | 5593.63 | 3482.22 | 5681.45 |
| AT2G39200 | 5179.32 | 5821.64 | 5037.85 | 5665.74 |
| AT3G56360 | 3523.14 | 5696.51 | 3522.84 | 5602.26 |
| AT1G15040 | 2677.05 | 5590.67 | 2974.84 | 5585.75 |
| AT5G66400 | 3990.79 | 4256.05 | 4625.69 | 5544.75 |
| AT4G01870 | 4589.86 | 5531.82 | 4415.21 | 5483.60 |
| AT5G56870 | 3710.66 | 5064.44 | 3959.70 | 5090.22 |
| AT3G19390 | 2282.54 | 5063.88 | 2733.05 | 5028.37 |
| AT5G06300 | 4016.04 | 4502.49 | 4312.07 | 4779.59 |
| AT1G68440 | 2879.57 | 4408.78 | 3220.60 | 4635.18 |
| AT1G10070 | 1991.74 | 4673.62 | 2354.02 | 4455.75 |
| AT5G20150 | 2916.04 | 3829.24 | 3543.14 | 4451.22 |
| AT1G23870 | 2774.60 | 3629.67 | 3635.51 | 4415.35 |
| AT3G47960 | 2989.02 | 3938.43 | 3321.11 | 4262.19 |
| AT5G47740 | 3367.67 | 3947.58 | 3614.82 | 4217.10 |
| AT2G23170 | 3558.08 | 4503.52 | 3485.93 | 4165.23 |
| AT4G38470 | 1408.69 | 3152.12 | 2007.53 | 4099.55 |
| AT2G19800 | 2246.77 | 4333.96 | 2200.99 | 3882.93 |
| AT5G67300 | 3211.67 | 3812.88 | 3290.90 | 3832.21 |
| AT3G61260 | 2826.59 | 4226.43 | 2752.02 | 3824.18 |
| AT2G38400 | 1970.17 | 3129.70 | 2516.19 | 3716.21 |
| AT1G54100 | 2555.41 | 3120.81 | 3004.69 | 3689.79 |
| AT5G49440 | 2727.51 | 3759.00 | 2516.28 | 3613.92 |
| AT1G67480 | 1002.02 | 3773.82 | 1059.32 | 3525.08 |
| AT1G64660 | 1905.16 | 3536.41 | 2134.11 | 3434.33 |
| AT1G25275 | 2905.62 | 3755.00 | 2568.03 | 3299.31 |
| AT4G33150 | 1814.47 | 2840.47 | 2230.03 | 3288.96 |
| AT3G04070 | 2724.93 | 3390.50 | 2797.02 | 3266.73 |
| AT5G57655 | 2290.70 | 2911.98 | 2555.48 | 3247.33 |
| AT5G43580 | 2427.72 | 3256.69 | 2635.41 | 3222.34 |
| AT4G35770 | 948.38 | 3140.55 | 1314.57 | 3177.19 |
| AT5G11090 | 2078.24 | 2784.47 | 2283.94 | 3085.78 |
| AT1G08830 | 2441.65 | 2922.21 | 2469.18 | 2780.36 |
| AT3G56240 | 2353.08 | 2907.75 | 2327.40 | 2728.18 |
| AT1G79340 | 2204.62 | 2609.32 | 2337.77 | 2721.73 |
| AT5G54500 | 2372.67 | 3095.73 | 2004.25 | 2690.71 |
| AT3G05200 | 1793.81 | 2231.06 | 1938.52 | 2553.69 |
| AT4G36040 | 1903.75 | 2772.28 | 1948.94 | 2551.54 |
| AT1G68620 | 1757.50 | 2432.63 | 1713.98 | 2503.30 |
| AT1G11260 | 1818.65 | 2621.83 | 1712.04 | 2398.40 |
| AT4G32950 | 481.13 | 2304.42 | 619.27 | 2368.04 |
| AT4G20860 | 1743.34 | 2314.59 | 1847.07 | 2193.24 |
| AT1G14330 | 1769.07 | 2184.54 | 1787.45 | 2156.24 |
| AT3G14990 | 1486.66 | 2353.45 | 1600.26 | 2108.65 |
| AT4G15550 | 1482.06 | 1895.48 | 1505.45 | 2052.15 |
| AT5G50200 | 1702.01 | 2185.46 | 1724.26 | 2040.88 |
| AT4G37790 | 1516.07 | 2019.80 | 1563.38 | 2034.09 |
| AT1G03090 | 1196.37 | 1905.17 | 1458.24 | 2014.86 |
| AT2G33150 | 1467.96 | 1678.65 | 1719.03 | 2011.98 |
| AT1G43160 | 1640.78 | 2089.38 | 1677.85 | 2004.44 |
| AT5G05340 | 1990.71 | 2455.06 | 1675.36 | 1997.69 |
| AT1G22360 | 1435.43 | 1834.51 | 1651.79 | 1940.94 |
| AT5G64260 | 1833.49 | 2167.71 | 1692.73 | 1935.14 |
| AT1G32460 | 1457.27 | 2439.73 | 1366.24 | 1929.38 |
| AT1G29400 | 1732.04 | 2012.76 | 1657.37 | 1917.29 |
| AT5G11520 | 1366.90 | 1831.79 | 1466.17 | 1910.76 |
| AT4G39780 | 1312.50 | 1899.95 | 1496.49 | 1897.93 |
| AT5G67310 | 1827.99 | 2284.68 | 1585.68 | 1860.24 |
| AT5G08350 | 73.19 | 1944.64 | 79.08 | 1798.98 |
| AT3G15450 | 1476.21 | 1901.72 | 1501.24 | 1773.58 |
| AT5G28610 | 1341.26 | 1888.26 | 1387.43 | 1761.45 |
| AT4G03510 | 953.82 | 1726.31 | 968.89 | 1759.19 |
| AT2G38750 | 1213.34 | 1600.58 | 1313.42 | 1695.27 |
| AT5G67320 | 1596.01 | 2100.45 | 1420.94 | 1678.29 |
| AT3G14770 | 643.54 | 1627.16 | 767.40 | 1620.43 |
| AT1G27100 | 1301.70 | 1680.26 | 1281.77 | 1598.90 |
| AT1G69890 | 1143.76 | 1882.02 | 1024.97 | 1556.08 |
| AT5G61600 | 1383.51 | 1762.18 | 1271.92 | 1552.54 |
| AT1G80460 | 1202.78 | 1535.00 | 1168.86 | 1548.39 |
| AT5G48430 | 1757.39 | 2322.30 | 1372.87 | 1508.49 |
| AT3G11410 | 1189.35 | 1363.58 | 1210.84 | 1479.68 |
| AT4G27260 | 1018.07 | 1484.79 | 1009.82 | 1464.57 |
| AT3G51730 | 907.42 | 1308.15 | 1001.97 | 1457.17 |
| AT1G04410 | 946.12 | 1162.96 | 1212.68 | 1441.19 |
| AT2G02800 | 1173.58 | 1632.63 | 1183.50 | 1407.36 |
| AT2G32660 | 1135.17 | 1410.73 | 1132.61 | 1400.63 |
| AT3G43430 | 905.85 | 1670.63 | 819.65 | 1400.22 |
| AT3G55450 | 1238.32 | 1609.73 | 1068.06 | 1354.31 |
| AT1G08930 | 1194.49 | 1381.44 | 1091.15 | 1306.69 |
| AT5G44380 | 1054.14 | 1584.83 | 983.22 | 1289.14 |
| AT3G52060 | 867.54 | 1171.26 | 825.77 | 1284.28 |
| AT3G15630 | 970.70 | 1691.80 | 902.42 | 1237.34 |
| AT1G08920 | 843.98 | 1132.36 | 964.58 | 1195.78 |
| AT1G30820 | 610.39 | 1245.12 | 725.50 | 1164.67 |
| AT4G34350 | 630.77 | 988.61 | 876.35 | 1164.52 |
| AT5G16110 | 798.11 | 1210.43 | 756.99 | 1139.91 |
| AT4G38060 | 885.30 | 1080.25 | 916.81 | 1109.34 |
| AT3G19930 | 669.32 | 1349.89 | 603.66 | 1089.31 |
| AT3G06850 | 705.39 | 1259.52 | 746.19 | 1072.72 |
| AT1G68410 | 917.29 | 1179.01 | 921.44 | 1054.98 |
| AT3G12320 | 768.10 | 957.25 | 887.47 | 1030.97 |
| AT1G18270 | 554.66 | 1117.09 | 589.44 | 1007.85 |
| AT4G15630 | 660.42 | 1000.90 | 631.83 | 1003.19 |
| AT1G15380 | 677.06 | 949.20 | 711.13 | 1002.44 |
| AT4G30490 | 790.90 | 982.97 | 819.52 | 992.49 |
| AT5G20250 | 295.01 | 1063.61 | 377.67 | 976.27 |
| AT3G45300 | 527.47 | 768.10 | 788.61 | 968.78 |
| AT3G15950 | 637.26 | 1011.75 | 601.23 | 948.72 |
| AT5G65110 | 645.46 | 1043.94 | 629.59 | 925.59 |
| AT3G46690 | 512.82 | 889.96 | 495.74 | 921.56 |
| AT2G39210 | 530.68 | 850.67 | 614.41 | 890.85 |
| AT5G41610 | 493.04 | 1077.30 | 399.49 | 882.34 |
| AT4G24220 | 595.64 | 978.37 | 543.62 | 877.62 |
| AT5G04040 | 492.68 | 886.80 | 594.52 | 877.26 |
| AT1G28130 | 569.60 | 844.63 | 506.85 | 876.67 |
| AT5G67420 | 206.65 | 326.20 | 733.39 | 876.59 |
| AT1G76990 | 511.46 | 794.92 | 531.69 | 869.96 |
| AT5G24530 | 533.88 | 826.44 | 559.74 | 845.58 |
| AT4G18340 | 257.55 | 1111.63 | 246.45 | 834.12 |
| AT5G10450 | 700.33 | 846.43 | 688.08 | 822.06 |
| AT3G17110 | 530.43 | 585.81 | 570.25 | 819.94 |
| AT2G32510 | 497.35 | 774.09 | 529.25 | 811.11 |
| AT1G29760 | 610.06 | 780.86 | 724.85 | 788.34 |
| AT1G22830 | 535.37 | 873.50 | 565.71 | 787.30 |
| AT2G30600 | 358.04 | 829.99 | 331.61 | 780.48 |
| AT1G22190 | 620.74 | 786.03 | 592.52 | 768.94 |
| AT1G58180 | 440.02 | 836.01 | 428.79 | 761.66 |
| AT2G31390 | 481.43 | 580.20 | 596.80 | 761.61 |
| AT3G29240 | 326.16 | 792.83 | 352.29 | 756.15 |
| AT3G49790 | 474.90 | 852.12 | 449.07 | 731.71 |
| AT2G38820 | 295.83 | 728.53 | 332.08 | 715.79 |
| AT1G08720 | 633.75 | 805.71 | 610.86 | 709.33 |
| AT4G01026 | 499.36 | 799.47 | 501.33 | 689.90 |
| AT1G26270 | 437.10 | 749.41 | 455.57 | 684.98 |
| AT4G21440 | 518.10 | 847.63 | 390.31 | 677.20 |
| AT5G54080 | 466.34 | 563.21 | 571.81 | 670.32 |
| AT1G62570 | 480.50 | 653.30 | 563.75 | 668.93 |
| AT1G76410 | 477.22 | 685.02 | 530.84 | 665.41 |
| AT4G32870 | 600.84 | 739.01 | 436.98 | 652.54 |
| AT5G45630 | 293.64 | 851.38 | 248.54 | 650.24 |
| AT3G51840 | 456.42 | 561.01 | 539.55 | 649.19 |
| AT1G55510 | 359.48 | 505.19 | 438.72 | 632.62 |
| AT1G76240 | 416.87 | 627.84 | 476.67 | 628.97 |
| AT3G16150 | 73.84 | 851.91 | 77.41 | 622.58 |
| AT5G40450 | 425.44 | 515.17 | 539.91 | 610.18 |
| AT2G23450 | 477.27 | 663.73 | 456.64 | 608.98 |
| AT5G49360 | 100.52 | 564.27 | 138.45 | 583.56 |
| AT4G10840 | 411.43 | 503.25 | 459.63 | 553.75 |
| AT5G15190 | 245.58 | 502.01 | 320.39 | 540.57 |
| AT2G44670 | 312.67 | 631.34 | 285.27 | 535.16 |
| AT3G61060 | 163.22 | 600.33 | 208.76 | 531.13 |
| AT2G12400 | 323.07 | 500.47 | 366.46 | 529.50 |
| AT3G13460 | 433.42 | 520.52 | 408.87 | 525.71 |
| AT1G06570 | 263.25 | 395.08 | 327.78 | 518.78 |
| AT2G26280 | 431.35 | 509.69 | 443.22 | 516.43 |
| AT5G04740 | 413.67 | 480.88 | 418.89 | 506.93 |
| AT2G14170 | 302.99 | 514.20 | 317.60 | 505.01 |
| AT1G02860 | 290.17 | 632.44 | 310.74 | 504.02 |
| AT4G13430 | 356.83 | 440.33 | 378.32 | 502.99 |
| AT1G72770 | 250.42 | 475.96 | 359.01 | 501.13 |
| AT1G55020 | 222.81 | 510.73 | 247.00 | 500.39 |
| AT3G54620 | 384.47 | 486.84 | 414.77 | 496.60 |
| AT1G65840 | 487.99 | 619.99 | 440.66 | 490.79 |
| AT3G54140 | 301.69 | 421.20 | 364.28 | 486.97 |
| AT4G39730 | 304.20 | 457.54 | 298.99 | 467.18 |
| AT4G17950 | 378.17 | 463.37 | 417.61 | 466.59 |
| AT4G01120 | 171.46 | 448.16 | 195.47 | 455.26 |
| AT1G01490 | 296.79 | 504.22 | 354.46 | 455.22 |
| AT1G16150 | 389.48 | 664.54 | 258.23 | 451.86 |
| AT3G57890 | 359.52 | 398.76 | 365.14 | 448.61 |
| AT3G23230 | 477.73 | 720.03 | 284.96 | 446.59 |
| AT3G51860 | 359.25 | 406.42 | 347.34 | 439.13 |
| AT1G61660 | 376.46 | 482.03 | 340.63 | 433.51 |
| AT2G39570 | 147.42 | 576.87 | 162.97 | 427.86 |
| AT1G67810 | 258.42 | 466.67 | 261.45 | 418.71 |
| AT1G63180 | 293.52 | 504.12 | 298.99 | 418.36 |
| AT5G16970 | 274.42 | 361.16 | 293.68 | 417.10 |
| AT5G63620 | 321.59 | 383.09 | 361.31 | 415.60 |
| AT4G29950 | 248.77 | 424.65 | 265.88 | 410.83 |
| AT3G46440 | 296.17 | 388.65 | 320.64 | 407.40 |
| AT3G01175 | 360.83 | 535.86 | 283.40 | 405.06 |
| AT3G17420 | 277.61 | 422.27 | 317.09 | 397.44 |
| AT1G66470 | 226.02 | 346.31 | 299.54 | 391.92 |
| AT3G46280 | 386.23 | 572.31 | 286.47 | 390.51 |
| AT3G57540 | 246.02 | 338.91 | 315.59 | 386.72 |
| AT3G53150 | 309.42 | 429.81 | 287.16 | 383.79 |
| AT1G03790 | 223.72 | 293.00 | 296.24 | 383.24 |
| AT1G61740 | 204.52 | 373.13 | 248.24 | 382.22 |
| AT5G61590 | 169.36 | 322.75 | 217.79 | 369.15 |
| AT4G23880 | 246.89 | 339.77 | 230.07 | 368.41 |
| AT4G15620 | 220.23 | 431.88 | 163.64 | 360.52 |
| AT5G64460 | 232.76 | 344.86 | 270.38 | 357.94 |
| AT1G75450 | 201.99 | 431.13 | 186.76 | 355.21 |
| AT2G15695 | 223.60 | 335.08 | 210.60 | 354.64 |
| AT3G17440 | 235.08 | 325.79 | 244.52 | 350.48 |
| AT3G20410 | 260.99 | 397.46 | 248.52 | 347.30 |
| AT3G19920 | 181.43 | 297.01 | 181.99 | 347.01 |
| AT2G27490 | 265.83 | 366.46 | 281.46 | 346.25 |
| AT1G75230 | 298.68 | 362.99 | 303.28 | 344.29 |
| AT5G37260 | 183.58 | 323.24 | 191.07 | 338.89 |
| AT3G48690 | 136.33 | 376.40 | 124.31 | 333.70 |
| AT5G06980 | 229.34 | 369.71 | 276.34 | 328.19 |
| AT4G28040 | 126.83 | 311.27 | 164.96 | 326.75 |
| AT1G35580 | 249.68 | 388.17 | 229.39 | 326.38 |
| AT5G24470 | 237.51 | 330.86 | 230.82 | 321.05 |
| AT4G14420 | 278.60 | 365.61 | 218.88 | 314.73 |
| AT2G25900 | 230.39 | 321.40 | 271.91 | 312.60 |
| AT5G18630 | 212.25 | 282.12 | 248.33 | 301.33 |
| AT5G13740 | 148.46 | 287.89 | 167.62 | 297.04 |
| AT1G03100 | 184.63 | 398.39 | 169.38 | 294.96 |
| AT1G49670 | 246.84 | 272.38 | 248.68 | 292.52 |
| AT1G54740 | 50.65 | 279.25 | 63.67 | 287.90 |
| AT3G03170 | 188.22 | 240.68 | 195.94 | 280.19 |
| AT1G67470 | 233.81 | 347.51 | 219.61 | 279.31 |
| AT1G06520 | 246.49 | 338.83 | 194.75 | 277.92 |
| AT1G56700 | 173.77 | 270.96 | 225.74 | 276.90 |
| AT3G13450 | 115.41 | 292.52 | 112.43 | 273.07 |
| AT1G03610 | 176.69 | 299.85 | 197.11 | 271.81 |
| AT3G14050 | 184.59 | 249.89 | 184.11 | 269.92 |
| AT5G46590 | 54.42 | 277.55 | 61.46 | 267.30 |
| AT1G11380 | 112.85 | 259.85 | 131.99 | 263.86 |
| AT5G66030 | 210.45 | 277.21 | 211.28 | 262.39 |
| AT2G43060 | 121.21 | 237.36 | 133.52 | 261.39 |
| AT4G30550 | 176.17 | 235.71 | 202.63 | 257.78 |
| AT1G56145 | 153.41 | 292.27 | 152.77 | 256.92 |
| AT1G19700 | 210.69 | 250.61 | 214.73 | 256.72 |
| AT2G17500 | 213.62 | 274.99 | 194.47 | 253.08 |
| AT4G03080 | 218.91 | 264.94 | 211.69 | 252.74 |
| AT4G24330 | 166.59 | 259.57 | 205.16 | 252.10 |
| AT5G18610 | 137.12 | 200.94 | 163.91 | 244.41 |
| AT5G43190 | 173.58 | 276.27 | 162.71 | 243.92 |
| AT3G11340 | 103.93 | 253.84 | 95.09 | 242.44 |
| AT1G69570 | 53.47 | 280.68 | 66.82 | 240.47 |
| AT5G16120 | 166.52 | 193.99 | 185.28 | 239.74 |
| AT1G03080 | 141.88 | 212.23 | 177.46 | 237.30 |
| AT5G47390 | 214.99 | 261.98 | 197.83 | 237.13 |
| AT5G02780 | 208.46 | 325.07 | 147.70 | 230.92 |
| AT1G08630 | 86.59 | 305.91 | 81.73 | 227.16 |
| AT2G22080 | 167.39 | 214.32 | 161.86 | 226.72 |
| AT3G61070 | 159.27 | 230.78 | 167.05 | 222.96 |
| AT5G49690 | 134.05 | 194.00 | 132.84 | 222.19 |
| AT4G15280 | 162.18 | 216.41 | 177.44 | 221.97 |
| AT1G48840 | 138.48 | 191.27 | 166.86 | 220.61 |
| AT1G23550 | 115.16 | 205.81 | 126.48 | 219.78 |
| AT3G52710 | 185.70 | 224.55 | 173.68 | 219.41 |
| AT4G26290 | 100.09 | 204.28 | 99.72 | 217.88 |
| AT1G66070 | 187.56 | 241.42 | 186.15 | 214.37 |
| AT1G71980 | 87.74 | 200.94 | 104.80 | 211.66 |
| AT5G27350 | 169.81 | 217.50 | 181.09 | 209.46 |
| AT4G30170 | 81.34 | 235.80 | 82.59 | 204.62 |
| AT1G76160 | 149.85 | 241.80 | 165.87 | 203.41 |
| AT3G16800 | 136.82 | 188.05 | 163.34 | 203.20 |
| AT4G15545 | 156.88 | 210.11 | 145.84 | 202.89 |
| AT2G29380 | 133.78 | 205.70 | 133.94 | 195.73 |
| AT3G05390 | 162.24 | 261.87 | 157.21 | 195.25 |
| AT4G32320 | 139.86 | 219.65 | 135.38 | 194.46 |
| AT1G23880 | 108.11 | 207.96 | 154.52 | 193.41 |
| AT5G43430 | 167.02 | 202.42 | 152.23 | 192.88 |
| AT5G02810 | 115.69 | 171.62 | 147.36 | 191.61 |
| AT4G33910 | 133.44 | 231.40 | 133.01 | 188.06 |
| AT3G16910 | 132.96 | 200.97 | 129.81 | 187.98 |
| AT4G37540 | 23.62 | 63.65 | 56.50 | 187.59 |
| AT5G07080 | 174.68 | 247.22 | 134.82 | 185.93 |
| AT5G24030 | 177.37 | 240.76 | 142.93 | 183.54 |
| AT3G57020 | 115.75 | 152.78 | 139.84 | 183.40 |
| AT1G17190 | 92.87 | 188.86 | 111.21 | 180.24 |
| AT3G14780 | 137.88 | 158.41 | 144.07 | 179.84 |
| AT4G14500 | 124.89 | 193.26 | 132.77 | 179.33 |
| AT1G08090 | 90.27 | 189.04 | 104.52 | 177.75 |
| AT3G60690 | 63.78 | 115.19 | 80.58 | 175.69 |
| AT3G55150 | 68.56 | 258.96 | 42.22 | 174.54 |
| AT5G16960 | 123.23 | 167.73 | 131.78 | 171.56 |
| AT2G46270 | 77.59 | 150.29 | 99.41 | 171.54 |
| AT5G17640 | 107.49 | 190.13 | 121.67 | 171.17 |
| AT3G20860 | 102.93 | 177.80 | 86.02 | 170.02 |
| AT3G45060 | 88.06 | 139.17 | 146.70 | 169.39 |
| AT1G67880 | 141.83 | 194.88 | 147.45 | 169.14 |
| AT5G26740 | 88.42 | 165.00 | 91.06 | 165.11 |
| AT5G43830 | 124.16 | 213.01 | 108.34 | 164.50 |
| AT1G32200 | 94.05 | 141.29 | 96.44 | 163.07 |
| AT5G04770 | 106.01 | 197.32 | 98.70 | 162.60 |
| AT5G18850 | 87.29 | 173.24 | 106.32 | 161.22 |
| AT4G17140 | 125.32 | 158.91 | 129.87 | 160.87 |
| AT3G15610 | 127.30 | 184.88 | 138.02 | 159.29 |
| AT1G18260 | 121.01 | 130.77 | 129.39 | 157.84 |
| AT4G39070 | 81.16 | 197.76 | 78.43 | 157.28 |
| AT1G13080 | 56.39 | 132.02 | 56.78 | 155.59 |
| AT4G27657 | 89.63 | 155.91 | 96.74 | 153.67 |
| AT1G68850 | 191.27 | 332.59 | 80.41 | 153.54 |
| AT3G22930 | 120.29 | 248.25 | 108.40 | 152.64 |
| AT4G37590 | 107.97 | 167.29 | 121.12 | 152.57 |
| AT5G59220 | 121.92 | 147.72 | 123.64 | 151.98 |
| AT4G35780 | 101.85 | 146.06 | 104.35 | 151.98 |
| AT1G31480 | 103.91 | 150.30 | 117.73 | 151.70 |
| AT5G57630 | 99.77 | 156.71 | 119.20 | 151.17 |
| AT1G21310 | 110.32 | 184.92 | 105.96 | 143.26 |
| AT4G32300 | 84.23 | 150.08 | 79.46 | 142.38 |
| AT1G66170 | 143.51 | 216.29 | 88.76 | 141.44 |
| AT5G27920 | 51.23 | 141.94 | 55.44 | 140.96 |
| AT2G40170 | 66.43 | 140.44 | 63.41 | 139.40 |
| AT5G13750 | 79.95 | 151.37 | 81.87 | 139.08 |
| AT5G65630 | 109.66 | 146.68 | 116.75 | 138.71 |
| AT4G32960 | 108.46 | 144.34 | 116.28 | 138.12 |
| AT3G47500 | 103.77 | 139.68 | 110.41 | 137.67 |
| AT5G03720 | 74.31 | 137.22 | 89.23 | 137.53 |
| AT4G36670 | 93.16 | 179.25 | 91.44 | 133.77 |
| AT4G20870 | 109.56 | 213.99 | 77.04 | 133.70 |
| AT5G56100 | 95.00 | 127.73 | 90.07 | 133.47 |
| AT5G18170 | 58.10 | 106.68 | 83.00 | 132.91 |
| AT2G44360 | 111.72 | 152.31 | 114.74 | 131.90 |
| AT5G61510 | 97.58 | 121.52 | 105.48 | 131.82 |
| AT3G14067 | 84.56 | 132.94 | 109.50 | 131.21 |
| AT5G23050 | 79.96 | 112.81 | 85.27 | 128.37 |
| AT1G09460 | 51.78 | 96.15 | 68.09 | 128.23 |
| AT5G60200 | 57.29 | 113.64 | 51.11 | 127.35 |
| AT3G62650 | 83.14 | 114.05 | 100.20 | 127.07 |
| AT5G56180 | 93.37 | 125.51 | 108.00 | 126.69 |
| AT1G61810 | 55.96 | 171.39 | 50.57 | 126.49 |
| AT4G36790 | 93.30 | 121.16 | 92.98 | 124.48 |
| AT2G28200 | 80.09 | 107.22 | 66.42 | 122.50 |
| AT5G18650 | 80.20 | 119.46 | 94.34 | 121.87 |
| AT1G66550 | 16.87 | 178.52 | 8.85 | 121.01 |
| AT2G43400 | 80.72 | 117.84 | 102.26 | 120.82 |
| AT4G24060 | 74.58 | 116.88 | 79.81 | 120.75 |
| AT1G60940 | 89.47 | 114.81 | 94.79 | 120.19 |
| AT5G13110 | 50.81 | 55.50 | 85.60 | 117.80 |
| AT1G73240 | 84.91 | 120.96 | 82.12 | 115.06 |
| AT3G47640 | 88.22 | 124.31 | 95.33 | 113.52 |
| AT1G79700 | 99.25 | 144.06 | 84.18 | 110.96 |
| AT5G67450 | 137.07 | 197.99 | 91.95 | 108.74 |
| AT4G01030 | 80.36 | 134.69 | 62.12 | 108.14 |
| AT5G07070 | 79.82 | 107.17 | 83.36 | 107.96 |
| AT3G54630 | 35.72 | 138.03 | 32.62 | 106.41 |
| AT5G57660 | 57.73 | 90.98 | 77.73 | 105.82 |
| AT5G10210 | 21.30 | 48.72 | 64.20 | 102.49 |
| AT3G29160 | 78.64 | 120.24 | 79.77 | 98.18 |
| AT1G63700 | 78.58 | 105.41 | 68.50 | 95.18 |
| AT4G37220 | 49.77 | 108.81 | 49.51 | 94.74 |
| AT5G05440 | 83.03 | 131.52 | 73.58 | 93.44 |
| AT3G56000 | 72.31 | 96.23 | 66.14 | 92.92 |
| AT2G19350 | 68.37 | 78.86 | 72.52 | 92.41 |
| AT4G31240 | 52.21 | 69.75 | 70.95 | 92.27 |
| AT4G38500 | 32.40 | 67.18 | 48.08 | 88.95 |
| AT1G75220 | 77.58 | 101.05 | 73.89 | 87.30 |
| AT2G19810 | 66.83 | 96.10 | 60.49 | 87.19 |
| AT3G49060 | 71.49 | 102.76 | 76.53 | 86.88 |
| AT4G36730 | 70.75 | 99.16 | 72.19 | 86.62 |
| AT1G57680 | 80.22 | 119.55 | 70.72 | 85.92 |
| AT1G52240 | 24.19 | 113.85 | 24.24 | 84.31 |
| AT2G39130 | 60.17 | 77.61 | 62.96 | 83.54 |
| AT1G15050 | 26.09 | 148.94 | 25.21 | 82.55 |
| AT1G07250 | 65.31 | 70.10 | 59.97 | 80.93 |
| AT1G28260 | 38.14 | 59.37 | 53.22 | 80.56 |
| AT3G06780 | 69.06 | 95.10 | 65.59 | 80.51 |
| AT1G79350 | 77.83 | 99.11 | 70.96 | 80.08 |
| AT1G14340 | 44.89 | 51.11 | 62.44 | 78.79 |
| AT5G49650 | 58.97 | 83.31 | 69.58 | 76.81 |
| AT1G20300 | 42.65 | 75.93 | 44.46 | 76.36 |
| AT2G39980 | 43.88 | 88.55 | 40.86 | 74.51 |
| AT5G58620 | 64.50 | 88.80 | 63.60 | 72.85 |
| AT2G22870 | 88.47 | 110.12 | 61.46 | 70.40 |
| AT3G15260 | 52.85 | 64.15 | 55.86 | 70.34 |
| AT1G75800 | 35.21 | 55.46 | 43.50 | 69.04 |
| AT3G02550 | 42.17 | 91.77 | 38.20 | 67.55 |
| AT1G18460 | 37.13 | 60.92 | 42.38 | 66.81 |
| AT5G13760 | 47.74 | 66.43 | 54.92 | 66.73 |
| AT1G26730 | 49.92 | 91.39 | 52.09 | 66.01 |
| AT2G35230 | 55.94 | 73.72 | 45.53 | 65.92 |
| AT3G14760 | 30.68 | 107.79 | 22.41 | 65.15 |
| AT3G50780 | 50.65 | 62.62 | 44.75 | 64.77 |
| AT1G69910 | 54.48 | 71.77 | 51.52 | 64.24 |
| AT5G39040 | 48.05 | 70.13 | 45.72 | 64.19 |
| AT3G51540 | 38.50 | 68.02 | 37.25 | 63.50 |
| AT2G41190 | 6.86 | 45.71 | 15.34 | 62.77 |
| AT5G20050 | 51.77 | 72.19 | 47.39 | 62.08 |
| AT1G32930 | 63.44 | 86.24 | 44.68 | 61.75 |
| AT2G01570 | 46.85 | 71.90 | 51.08 | 61.62 |
| AT3G14740 | 26.43 | 87.61 | 24.95 | 58.46 |
| AT3G24520 | 35.14 | 63.23 | 34.03 | 58.25 |
| AT2G40420 | 40.27 | 53.19 | 45.52 | 58.14 |
| AT1G18330 | 26.34 | 54.45 | 37.77 | 57.86 |
| AT3G49940 | 23.55 | 32.95 | 38.48 | 52.17 |
| AT3G57420 | 29.77 | 51.55 | 31.01 | 50.89 |
| AT3G16170 | 38.77 | 48.49 | 44.68 | 50.20 |
| AT5G47560 | 17.64 | 31.89 | 28.49 | 49.55 |
| AT3G27690 | 14.62 | 72.47 | 13.43 | 49.52 |
| AT4G33420 | 47.88 | 90.04 | 43.70 | 48.00 |
| AT2G19320 | 24.44 | 42.04 | 20.39 | 47.63 |
| AT1G66890 | 11.36 | 50.55 | 10.69 | 46.58 |
| AT3G14750 | 32.16 | 55.23 | 29.39 | 45.87 |
| AT4G38490 | 23.46 | 40.16 | 28.81 | 45.63 |
| AT2G26600 | 34.19 | 45.31 | 33.79 | 44.77 |
| AT3G54960 | 24.86 | 44.56 | 25.62 | 43.05 |
| AT1G08980 | 34.96 | 47.23 | 25.50 | 42.02 |
| AT3G13965 | 35.28 | 45.80 | 27.62 | 41.19 |
| AT2G02040 | 31.00 | 37.83 | 30.63 | 40.12 |
| AT1G67070 | 21.84 | 44.07 | 19.20 | 38.23 |
| AT5G47240 | 8.75 | 37.13 | 10.66 | 37.97 |
| AT1G67510 | 31.53 | 52.76 | 20.67 | 37.92 |
| AT5G06690 | 32.61 | 57.90 | 29.49 | 36.67 |
| AT1G06560 | 27.24 | 35.93 | 28.73 | 34.64 |
| AT5G19090 | 19.12 | 32.24 | 22.01 | 34.38 |
| AT1G64670 | 13.42 | 38.93 | 13.04 | 33.64 |
| AT4G01330 | 23.71 | 37.06 | 27.66 | 33.62 |
| AT5G59590 | 26.63 | 34.86 | 25.22 | 33.14 |
| AT3G22920 | 27.78 | 61.59 | 17.92 | 32.41 |
| AT4G38340 | 14.34 | 15.20 | 19.22 | 30.49 |
| AT4G38480 | 10.86 | 22.11 | 13.01 | 30.31 |
| AT1G15060 | 21.88 | 31.42 | 23.75 | 29.48 |
| AT2G03220 | 18.19 | 29.62 | 19.33 | 27.68 |
| AT1G10060 | 6.06 | 19.90 | 6.33 | 26.91 |
| AT1G22400 | 19.68 | 27.72 | 19.28 | 25.25 |
| AT3G15440 | 17.92 | 24.49 | 18.42 | 24.07 |
| AT4G23870 | 10.43 | 19.24 | 9.65 | 23.39 |
| AT3G15620 | 15.25 | 34.17 | 11.40 | 22.99 |
| AT2G02700 | 18.77 | 35.42 | 22.59 | 22.72 |
| AT5G52250 | 13.84 | 20.92 | 14.12 | 22.51 |
| AT1G64010 | 13.47 | 26.14 | 14.03 | 22.13 |
| AT5G67440 | 12.57 | 24.98 | 12.56 | 22.07 |
| AT5G03550 | 15.43 | 19.74 | 13.92 | 21.98 |
| AT2G37440 | 10.35 | 21.93 | 11.17 | 21.62 |
| AT1G42480 | 17.86 | 25.22 | 17.67 | 21.38 |
| AT4G27480 | 11.87 | 20.41 | 11.65 | 21.02 |
| AT1G03870 | 6.85 | 36.12 | 6.04 | 21.00 |
| AT3G15650 | 11.63 | 25.13 | 9.63 | 20.65 |
| AT3G02150 | 16.33 | 22.41 | 16.55 | 20.49 |
| AT1G10560 | 13.15 | 18.66 | 15.05 | 20.25 |
| AT5G20885 | 8.36 | 11.31 | 13.58 | 20.01 |
| AT1G30900 | 14.17 | 22.25 | 14.04 | 18.91 |
| AT5G51850 | 10.59 | 15.80 | 13.04 | 17.91 |
| AT1G76185 | 9.06 | 12.95 | 9.57 | 17.54 |
| AT1G51820 | 14.48 | 29.13 | 11.64 | 17.32 |
| AT3G19400 | 11.02 | 21.42 | 10.14 | 17.18 |
| AT5G63800 | 13.42 | 20.95 | 14.00 | 16.50 |
| AT3G52490 | 9.07 | 12.77 | 10.98 | 15.69 |
| AT2G03740 | 7.17 | 24.53 | 5.19 | 15.60 |
| AT2G28120 | 7.11 | 14.95 | 6.95 | 15.21 |
| AT3G03470 | 10.74 | 11.23 | 11.17 | 13.91 |
| AT3G60510 | 9.09 | 17.53 | 10.61 | 13.77 |
| AT1G68400 | 11.34 | 20.12 | 10.76 | 13.07 |
| AT5G01590 | 8.10 | 15.68 | 7.19 | 11.21 |
| AT1G12080 | 5.10 | 19.09 | 5.14 | 10.96 |
| AT2G31380 | 10.73 | 17.66 | 9.54 | 10.27 |
| AT1G11780 | 7.50 | 8.31 | 7.75 | 10.21 |
| AT1G63710 | 6.48 | 11.95 | 6.23 | 9.70 |
| AT4G16690 | 6.14 | 8.67 | 6.33 | 9.51 |
| AT3G01270 | 7.93 | 13.97 | 6.75 | 9.12 |
| AT2G01860 | 6.16 | 8.89 | 6.91 | 9.11 |
| AT5G03350 | 6.09 | 8.50 | 5.72 | 8.74 |
| AT2G10640 | 7.04 | 14.98 | 5.78 | 8.73 |
| AT4G01110 | 7.63 | 8.89 | 7.20 | 8.58 |
| AT3G30396 | 7.82 | 14.39 | 6.03 | 8.50 |
| AT3G18980 | 7.17 | 8.50 | 7.11 | 8.38 |
| AT5G04310 | 5.80 | 7.82 | 5.66 | 8.19 |
| AT1G20340 | 4.92 | 16.33 | 4.92 | 8.19 |
| AT4G19810 | 6.62 | 7.83 | 6.37 | 7.42 |
| AT1G03600 | 5.81 | 7.32 | 5.90 | 6.94 |
| AT2G28630 | 6.31 | 12.16 | 5.64 | 6.79 |
| AT4G38200 | 5.45 | 5.78 | 5.70 | 6.77 |
| AT3G28510 | 5.31 | 6.30 | 5.30 | 6.55 |
| AT1G02670 | 4.97 | 7.19 | 5.44 | 6.53 |
| AT5G04630 | 5.19 | 5.07 | 5.16 | 6.43 |
| AT3G24310 | 5.10 | 5.34 | 5.26 | 6.31 |
| AT2G41200 | 5.07 | 5.59 | 4.99 | 5.81 |
| B. Genes | | | | |
| that are | | | | |
| down-regulated | | | | |
| by DEX | | | | |
| (FDR < 0.05) | | | | |
| AT2G38470 | 10594.94 | 9805.00 | 10690.91 | 9439.25 |
| AT3G57450 | 10275.67 | 9151.09 | 9958.55 | 8270.15 |
| AT3G45640 | 8895.22 | 8082.87 | 8991.34 | 7649.50 |
| AT2G41730 | 7745.15 | 7011.42 | 7278.40 | 6457.43 |
| AT4G30280 | 7638.56 | 7227.50 | 7735.48 | 6672.97 |
| AT2G38870 | 7550.52 | 5944.54 | 6578.59 | 5449.26 |
| AT5G64310 | 7247.82 | 6331.15 | 7483.09 | 6501.53 |
| AT5G02230 | 7230.54 | 6000.23 | 7098.06 | 5757.55 |
| AT1G30370 | 7198.83 | 6096.88 | 6392.67 | 4996.87 |
| AT2G35980 | 6887.25 | 5915.12 | 6900.24 | 6080.70 |
| AT2G17660 | 6519.25 | 6035.21 | 7218.16 | 6322.89 |
| AT1G14540 | 6503.27 | 5600.91 | 5905.89 | 4876.12 |
| AT5G13190 | 6327.96 | 5777.02 | 6277.25 | 5641.66 |
| AT4G12720 | 5417.69 | 4831.47 | 5626.66 | 4720.71 |
| AT3G06490 | 5298.66 | 4516.36 | 5209.36 | 4230.12 |
| AT5G19240 | 5206.39 | 4093.63 | 4888.19 | 3710.43 |
| AT1G14550 | 5125.17 | 3201.22 | 3718.19 | 2242.62 |
| AT1G78100 | 4689.46 | 3678.75 | 4742.38 | 3865.18 |
| AT4G34150 | 4607.37 | 4291.35 | 4572.67 | 3996.05 |
| AT2G27390 | 4566.43 | 3837.18 | 4464.66 | 3801.81 |
| AT4G08850 | 4428.08 | 4007.17 | 4267.77 | 3829.57 |
| AT1G56060 | 4412.46 | 3059.50 | 3859.42 | 2460.41 |
| AT3G52400 | 4286.43 | 3807.93 | 4148.80 | 3653.63 |
| AT4G40040 | 4135.05 | 3616.24 | 3965.38 | 3614.05 |
| AT4G32020 | 3994.28 | 3119.61 | 3736.47 | 3114.00 |
| AT3G53730 | 3945.81 | 3259.21 | 4221.20 | 3631.17 |
| AT5G08240 | 3875.35 | 3274.32 | 3788.89 | 3294.04 |
| AT3G62720 | 3800.72 | 3410.65 | 3918.77 | 3158.22 |
| AT1G73010 | 3512.54 | 2948.47 | 4400.95 | 3492.79 |
| AT1G70130 | 3413.86 | 2346.61 | 3432.51 | 2378.90 |
| AT5G47910 | 3328.40 | 3079.49 | 3463.66 | 2840.76 |
| AT4G02380 | 3292.06 | 2079.41 | 3198.37 | 2088.63 |
| AT2G23270 | 3149.67 | 1677.37 | 2305.40 | 1208.21 |
| AT5G41810 | 3111.61 | 2679.01 | 3039.98 | 2659.79 |
| AT4G17230 | 3054.74 | 2738.50 | 3105.45 | 2656.17 |
| AT2G30130 | 2997.40 | 2304.29 | 3366.78 | 2457.53 |
| AT2G22500 | 2981.76 | 2536.55 | 3104.64 | 2641.18 |
| AT3G02800 | 2956.62 | 2435.58 | 2501.23 | 2086.28 |
| AT2G31880 | 2880.46 | 2387.48 | 2754.07 | 2290.80 |
| AT4G11360 | 2822.28 | 2152.72 | 2401.20 | 1756.26 |
| AT3G21070 | 2814.10 | 2300.53 | 2588.43 | 2215.90 |
| AT1G06760 | 2776.71 | 2378.31 | 2853.96 | 2519.50 |
| AT1G51920 | 2773.17 | 1979.42 | 2214.57 | 1517.34 |
| AT3G24550 | 2722.50 | 2655.04 | 2851.78 | 2564.13 |
| AT3G02880 | 2715.25 | 2563.73 | 2713.98 | 2352.20 |
| AT5G51190 | 2664.80 | 2220.62 | 2418.21 | 1944.26 |
| AT1G11210 | 2645.36 | 2160.38 | 3164.87 | 2356.86 |
| AT2G06050 | 2616.60 | 2165.16 | 2711.72 | 2128.88 |
| AT2G01450 | 2579.51 | 2177.06 | 2716.31 | 2140.39 |
| AT5G44610 | 2554.63 | 2138.70 | 2434.61 | 1936.00 |
| AT5G62350 | 2356.45 | 1655.92 | 2504.33 | 1559.98 |
| AT4G22470 | 2292.25 | 1969.35 | 2081.97 | 1738.58 |
| AT2G22470 | 2273.65 | 1776.05 | 2061.53 | 1672.60 |
| AT1G52200 | 2223.03 | 1509.49 | 1794.15 | 1240.12 |
| AT4G39260 | 2222.20 | 1848.46 | 2425.54 | 2171.34 |
| AT5G66070 | 2187.98 | 1822.99 | 2143.00 | 1836.79 |
| AT4G01850 | 2159.94 | 1177.09 | 1656.44 | 936.20 |
| AT4G37910 | 2039.79 | 1589.72 | 1708.63 | 1426.12 |
| AT4G24160 | 1998.33 | 1756.43 | 1899.18 | 1586.29 |
| AT4G32060 | 1969.28 | 1557.50 | 1840.41 | 1513.36 |
| AT2G19570 | 1967.42 | 1602.44 | 1658.96 | 1249.48 |
| AT5G61210 | 1920.02 | 1836.03 | 1854.71 | 1498.34 |
| AT5G07310 | 1917.88 | 1485.37 | 2045.93 | 1539.41 |
| AT1G13340 | 1867.19 | 1491.57 | 1944.69 | 1536.05 |
| AT2G17220 | 1821.13 | 1578.15 | 1680.18 | 1256.00 |
| AT1G80820 | 1802.73 | 1554.35 | 1839.63 | 1563.73 |
| AT3G13650 | 1722.32 | 901.05 | 1328.90 | 762.96 |
| AT5G48540 | 1708.97 | 1409.01 | 1678.11 | 1375.50 |
| AT1G04440 | 1701.41 | 1560.40 | 1762.71 | 1487.92 |
| AT3G55960 | 1694.09 | 1446.07 | 1693.87 | 1399.27 |
| AT4G30290 | 1653.42 | 1210.90 | 2032.78 | 1267.09 |
| AT4G28350 | 1653.13 | 1171.60 | 1439.88 | 1176.22 |
| AT3G11820 | 1600.50 | 1325.60 | 1566.12 | 1278.85 |
| AT1G59910 | 1591.72 | 1364.44 | 1646.71 | 1393.59 |
| AT5G07620 | 1569.64 | 1126.48 | 1339.31 | 1038.34 |
| AT5G44070 | 1567.49 | 1174.12 | 1195.05 | 1001.80 |
| AT3G17020 | 1555.88 | 1411.42 | 1574.25 | 1369.51 |
| AT3G59080 | 1536.13 | 1236.25 | 1350.16 | 1102.62 |
| AT3G61390 | 1524.35 | 1139.96 | 1463.77 | 830.00 |
| AT5G60680 | 1522.99 | 1052.96 | 1329.86 | 1009.48 |
| AT4G22820 | 1520.78 | 1316.01 | 1544.71 | 1311.00 |
| AT4G40030 | 1509.28 | 1144.40 | 1388.30 | 1028.36 |
| AT2G28570 | 1453.74 | 1102.83 | 1338.15 | 1081.58 |
| AT1G16670 | 1432.88 | 1244.29 | 1390.31 | 1188.06 |
| AT1G55920 | 1416.03 | 1028.24 | 1171.04 | 960.14 |
| AT5G39670 | 1332.60 | 1026.04 | 1354.50 | 999.29 |
| AT2G25735 | 1322.34 | 1095.95 | 1075.97 | 794.10 |
| AT1G28190 | 1320.00 | 1083.64 | 1321.61 | 1112.49 |
| AT1G72060 | 1292.51 | 1074.21 | 1075.49 | 836.87 |
| AT5G62390 | 1283.87 | 1092.32 | 1285.84 | 1090.86 |
| AT3G18250 | 1276.11 | 843.37 | 941.65 | 678.63 |
| AT4G18880 | 1273.25 | 1066.78 | 1254.86 | 948.83 |
| AT3G49720 | 1223.58 | 1045.45 | 1140.05 | 960.26 |
| AT2G25250 | 1156.23 | 798.81 | 1091.29 | 789.02 |
| AT1G02400 | 1125.76 | 824.28 | 1042.52 | 815.59 |
| AT3G50900 | 1123.55 | 910.84 | 1304.73 | 953.10 |
| AT1G17370 | 1117.83 | 820.42 | 998.40 | 784.41 |
| AT5G65020 | 1116.90 | 955.98 | 1226.70 | 1082.21 |
| AT4G25030 | 1106.98 | 910.25 | 898.93 | 760.64 |
| AT5G49620 | 1094.51 | 923.27 | 1155.71 | 887.29 |
| AT5G66880 | 1075.21 | 846.26 | 1061.06 | 939.67 |
| AT4G34180 | 1054.26 | 916.35 | 957.82 | 882.79 |
| AT3G22160 | 1027.28 | 773.11 | 897.50 | 660.04 |
| AT3G10640 | 1011.64 | 891.22 | 960.27 | 772.48 |
| AT5G58110 | 1009.27 | 882.17 | 1068.85 | 822.53 |
| AT1G72070 | 991.01 | 759.65 | 756.53 | 543.38 |
| AT2G26380 | 986.98 | 727.38 | 841.17 | 548.62 |
| AT5G06720 | 979.96 | 470.49 | 598.64 | 278.98 |
| AT3G52360 | 912.90 | 699.55 | 1017.10 | 882.60 |
| AT4G30470 | 894.34 | 752.07 | 1057.46 | 688.34 |
| AT4G37180 | 889.54 | 672.72 | 918.95 | 755.82 |
| AT5G57340 | 881.06 | 713.57 | 951.94 | 693.08 |
| AT3G44720 | 874.87 | 704.84 | 803.58 | 638.68 |
| AT1G18210 | 865.92 | 721.87 | 841.77 | 668.69 |
| AT4G37900 | 847.54 | 719.50 | 577.15 | 462.63 |
| AT4G38420 | 832.76 | 469.66 | 572.49 | 293.99 |
| AT3G09020 | 817.18 | 715.83 | 739.90 | 575.96 |
| AT5G26030 | 800.07 | 590.54 | 678.60 | 552.31 |
| AT3G08760 | 796.60 | 564.43 | 743.38 | 551.95 |
| AT3G21230 | 790.87 | 554.21 | 669.69 | 431.90 |
| AT1G32350 | 774.03 | 454.60 | 499.64 | 344.20 |
| AT2G32030 | 770.33 | 558.70 | 609.13 | 426.36 |
| AT5G60350 | 757.40 | 531.49 | 814.90 | 509.35 |
| AT1G51915 | 752.57 | 369.57 | 551.41 | 272.93 |
| AT1G09920 | 750.89 | 629.51 | 570.88 | 531.45 |
| AT2G39660 | 749.80 | 565.80 | 707.07 | 492.75 |
| AT1G78340 | 729.18 | 554.44 | 642.19 | 535.97 |
| AT3G54150 | 727.23 | 552.33 | 660.76 | 486.37 |
| AT5G37770 | 718.69 | 580.26 | 685.02 | 541.13 |
| AT1G20510 | 706.29 | 597.83 | 783.87 | 542.51 |
| AT2G19190 | 685.28 | 400.28 | 553.52 | 376.68 |
| AT1G18890 | 678.67 | 590.44 | 785.53 | 608.15 |
| AT5G14930 | 677.86 | 497.36 | 720.33 | 477.00 |
| AT3G54200 | 669.01 | 578.02 | 677.09 | 506.17 |
| AT1G73510 | 666.96 | 518.32 | 417.74 | 307.73 |
| AT4G31780 | 661.82 | 508.60 | 656.34 | 477.88 |
| AT3G05490 | 657.57 | 387.27 | 502.10 | 374.36 |
| AT1G63830 | 650.19 | 546.39 | 642.66 | 480.94 |
| AT3G28580 | 647.41 | 558.14 | 727.50 | 526.40 |
| AT5G39680 | 642.29 | 368.12 | 352.34 | 206.80 |
| AT4G24390 | 636.82 | 379.19 | 424.50 | 269.58 |
| AT5G42830 | 630.34 | 319.86 | 363.41 | 229.22 |
| AT4G28085 | 624.10 | 500.53 | 545.49 | 449.97 |
| AT1G09940 | 619.64 | 520.36 | 623.27 | 495.64 |
| AT2G24180 | 614.05 | 486.07 | 674.28 | 520.77 |
| AT2G26290 | 611.67 | 428.70 | 605.78 | 441.57 |
| AT3G04120 | 598.45 | 487.62 | 571.67 | 505.88 |
| AT4G37730 | 590.41 | 387.61 | 477.88 | 298.68 |
| AT1G51620 | 589.71 | 397.80 | 544.85 | 392.83 |
| AT4G30530 | 586.26 | 446.12 | 544.76 | 414.90 |
| AT2G20960 | 582.08 | 455.39 | 507.30 | 397.68 |
| AT4G33300 | 577.05 | 444.58 | 530.56 | 412.41 |
| AT3G10630 | 572.60 | 447.96 | 428.14 | 305.24 |
| AT1G19220 | 567.55 | 359.63 | 469.34 | 391.68 |
| AT1G74590 | 566.35 | 322.51 | 478.94 | 298.72 |
| AT2G42350 | 552.15 | 400.88 | 505.70 | 405.11 |
| AT2G26190 | 540.73 | 404.79 | 481.87 | 356.94 |
| AT2G39110 | 538.72 | 429.26 | 558.14 | 369.80 |
| AT1G11310 | 537.13 | 491.58 | 514.83 | 415.15 |
| AT2G41630 | 535.84 | 443.16 | 541.46 | 421.65 |
| AT3G47550 | 527.25 | 450.86 | 543.54 | 436.06 |
| AT4G00330 | 517.44 | 441.24 | 499.60 | 354.96 |
| AT2G38830 | 513.49 | 403.57 | 440.46 | 328.92 |
| AT4G37940 | 506.95 | 427.75 | 507.13 | 447.15 |
| AT3G08710 | 506.90 | 409.13 | 464.95 | 366.87 |
| AT5G62630 | 505.16 | 417.40 | 449.98 | 328.76 |
| AT5G51390 | 500.46 | 316.95 | 473.91 | 283.61 |
| AT2G21120 | 490.38 | 428.08 | 506.41 | 417.25 |
| AT3G55630 | 480.92 | 339.84 | 277.84 | 198.71 |
| AT5G41100 | 479.59 | 388.13 | 397.73 | 320.80 |
| AT2G43000 | 476.73 | 217.41 | 281.93 | 127.29 |
| AT4G11350 | 473.60 | 370.33 | 422.41 | 394.08 |
| AT4G16780 | 469.56 | 300.33 | 378.03 | 236.91 |
| AT5G04720 | 448.61 | 368.00 | 406.96 | 337.63 |
| AT2G46140 | 439.56 | 347.42 | 407.54 | 279.81 |
| AT4G36900 | 437.23 | 362.76 | 496.33 | 369.50 |
| AT2G42430 | 436.12 | 313.08 | 401.95 | 295.15 |
| AT5G59510 | 427.55 | 250.69 | 417.49 | 218.37 |
| AT2G47130 | 418.63 | 310.91 | 388.64 | 238.18 |
| AT3G48090 | 417.97 | 371.87 | 453.22 | 357.78 |
| AT4G18890 | 417.22 | 378.73 | 425.34 | 345.77 |
| AT3G61850 | 416.47 | 307.85 | 502.52 | 311.56 |
| AT2G39700 | 415.94 | 312.42 | 314.64 | 262.48 |
| AT4G39890 | 413.32 | 343.49 | 419.01 | 295.70 |
| AT5G59480 | 408.82 | 251.29 | 324.14 | 202.63 |
| AT5G45750 | 402.86 | 343.76 | 360.93 | 285.44 |
| AT5G60250 | 401.41 | 302.26 | 322.78 | 233.26 |
| AT3G09270 | 395.41 | 298.12 | 336.49 | 238.53 |
| AT1G71450 | 394.30 | 191.87 | 215.77 | 136.56 |
| AT1G10160 | 384.80 | 242.75 | 234.68 | 206.77 |
| AT1G65690 | 384.45 | 291.73 | 338.94 | 280.36 |
| AT1G24140 | 376.60 | 282.18 | 369.70 | 244.70 |
| AT4G02200 | 375.43 | 306.60 | 344.27 | 252.99 |
| AT4G29670 | 374.13 | 285.58 | 360.31 | 292.47 |
| AT4G14368 | 372.74 | 299.65 | 250.04 | 185.77 |
| AT1G34750 | 371.50 | 331.40 | 383.80 | 302.44 |
| AT5G54170 | 368.19 | 277.24 | 379.24 | 282.00 |
| AT4G31000 | 366.31 | 244.84 | 283.94 | 217.10 |
| AT5G12880 | 364.45 | 296.84 | 344.63 | 228.53 |
| AT1G79160 | 359.73 | 250.73 | 377.09 | 258.36 |
| AT1G18860 | 355.43 | 239.05 | 237.25 | 162.56 |
| AT2G17120 | 354.39 | 243.88 | 280.12 | 224.50 |
| AT5G66640 | 352.37 | 224.36 | 297.84 | 170.23 |
| AT3G54040 | 352.28 | 235.31 | 288.67 | 169.22 |
| AT5G24620 | 349.85 | 279.62 | 286.06 | 257.58 |
| AT4G23010 | 346.37 | 284.66 | 326.72 | 216.42 |
| AT1G70530 | 330.15 | 264.52 | 340.91 | 262.10 |
| AT4G01720 | 329.46 | 195.87 | 290.78 | 169.55 |
| AT2G26560 | 328.67 | 217.69 | 238.05 | 148.38 |
| AT2G19710 | 321.11 | 275.00 | 305.59 | 252.88 |
| AT3G28740 | 320.29 | 195.94 | 326.46 | 209.90 |
| AT4G21390 | 318.39 | 254.61 | 322.11 | 249.73 |
| AT3G55950 | 314.00 | 208.52 | 276.03 | 198.12 |
| AT5G65870 | 313.09 | 207.66 | 295.96 | 209.03 |
| AT1G53430 | 311.41 | 218.88 | 263.74 | 162.28 |
| AT1G57630 | 301.78 | 179.80 | 292.68 | 189.47 |
| AT5G01540 | 296.89 | 218.77 | 286.07 | 206.48 |
| AT5G53130 | 290.17 | 253.21 | 273.00 | 217.14 |
| AT1G75540 | 289.25 | 229.29 | 284.86 | 267.65 |
| AT2G16430 | 288.37 | 242.09 | 340.20 | 274.37 |
| AT2G24240 | 285.10 | 179.59 | 310.84 | 197.68 |
| AT2G47140 | 274.18 | 144.79 | 162.65 | 85.14 |
| AT4G30210 | 271.35 | 213.19 | 253.06 | 190.97 |
| AT4G39940 | 263.87 | 201.21 | 161.80 | 131.95 |
| AT3G21080 | 263.37 | 158.47 | 191.66 | 96.73 |
| AT3G25070 | 260.11 | 185.94 | 248.21 | 168.51 |
| AT1G17310 | 259.77 | 180.69 | 208.28 | 171.07 |
| AT3G52430 | 259.01 | 182.07 | 316.62 | 174.16 |
| AT3G05510 | 254.46 | 156.80 | 167.87 | 152.16 |
| AT1G07130 | 252.68 | 188.29 | 259.23 | 185.23 |
| AT4G12070 | 251.34 | 182.24 | 238.72 | 212.56 |
| AT3G29670 | 245.29 | 195.88 | 260.11 | 214.41 |
| AT5G24430 | 242.79 | 172.19 | 249.47 | 172.19 |
| AT5G44350 | 237.68 | 182.15 | 249.92 | 175.38 |
| AT3G02790 | 237.46 | 154.39 | 218.06 | 166.72 |
| AT3G03020 | 235.62 | 167.21 | 208.94 | 173.60 |
| AT4G40020 | 233.07 | 172.14 | 187.39 | 145.78 |
| AT3G43250 | 230.33 | 168.91 | 216.98 | 138.94 |
| AT5G22530 | 227.62 | 149.07 | 210.52 | 102.89 |
| AT2G01150 | 226.39 | 183.45 | 300.00 | 200.26 |
| AT3G59900 | 224.19 | 143.94 | 119.47 | 108.04 |
| AT2G27690 | 223.63 | 173.37 | 229.40 | 140.60 |
| AT5G40010 | 223.44 | 149.11 | 179.26 | 112.00 |
| AT3G20510 | 220.97 | 185.82 | 197.76 | 157.64 |
| AT1G18570 | 215.25 | 167.37 | 173.04 | 121.02 |
| AT1G07000 | 212.12 | 189.78 | 224.52 | 166.21 |
| AT1G61560 | 206.08 | 111.67 | 134.07 | 78.72 |
| AT5G46710 | 204.13 | 115.24 | 178.68 | 98.07 |
| AT1G08510 | 202.66 | 158.09 | 182.44 | 166.13 |
| AT3G11840 | 200.71 | 146.58 | 164.44 | 123.71 |
| AT4G00080 | 200.58 | 139.69 | 241.23 | 160.09 |
| AT1G61370 | 198.89 | 161.66 | 184.72 | 130.95 |
| AT5G43520 | 196.01 | 137.87 | 113.86 | 85.56 |
| AT3G07390 | 194.86 | 130.69 | 122.34 | 91.62 |
| AT3G23090 | 187.47 | 130.73 | 152.74 | 118.35 |
| AT2G44090 | 187.45 | 138.06 | 158.65 | 115.52 |
| AT3G47380 | 184.44 | 82.64 | 149.15 | 70.09 |
| AT4G11850 | 175.57 | 124.51 | 143.86 | 117.19 |
| AT3G19630 | 175.34 | 126.04 | 183.13 | 146.21 |
| AT2G41890 | 172.57 | 103.33 | 202.75 | 115.01 |
| AT3G16030 | 172.35 | 117.74 | 137.15 | 97.15 |
| AT5G22690 | 170.36 | 144.46 | 158.94 | 116.55 |
| AT1G74870 | 166.63 | 95.53 | 99.13 | 70.00 |
| AT1G73066 | 165.80 | 111.21 | 123.41 | 95.52 |
| AT1G05060 | 165.30 | 80.42 | 131.75 | 65.50 |
| AT1G44830 | 163.47 | 72.94 | 126.20 | 56.04 |
| AT3G14360 | 159.92 | 70.48 | 109.23 | 66.97 |
| AT1G07520 | 159.28 | 135.21 | 149.96 | 112.75 |
| AT4G01700 | 158.84 | 88.10 | 131.39 | 73.00 |
| AT5G10400 | 158.79 | 103.88 | 124.58 | 86.10 |
| AT3G63390 | 157.00 | 98.40 | 107.62 | 104.12 |
| AT2G11520 | 148.26 | 116.99 | 126.40 | 105.06 |
| AT3G53130 | 146.81 | 130.51 | 175.82 | 112.17 |
| AT2G34930 | 144.25 | 81.36 | 130.87 | 61.45 |
| AT1G29250 | 140.47 | 89.92 | 101.42 | 95.01 |
| AT1G30040 | 140.10 | 84.66 | 120.28 | 64.56 |
| AT2G39530 | 137.58 | 83.44 | 84.55 | 50.39 |
| AT1G32690 | 137.33 | 97.19 | 110.85 | 84.09 |
| AT2G42360 | 137.30 | 82.50 | 142.29 | 78.23 |
| AT2G22680 | 134.47 | 104.98 | 141.50 | 109.21 |
| AT3G02770 | 133.37 | 110.31 | 139.56 | 90.55 |
| AT5G57500 | 132.87 | 62.88 | 78.12 | 45.79 |
| AT2G37940 | 132.34 | 112.90 | 132.46 | 114.85 |
| AT4G21780 | 128.86 | 99.03 | 110.50 | 78.21 |
| AT1G80530 | 127.35 | 88.43 | 128.13 | 70.73 |
| AT5G62680 | 127.34 | 88.09 | 107.42 | 78.22 |
| AT1G66090 | 124.24 | 84.29 | 110.97 | 71.78 |
| AT1G48320 | 123.74 | 65.39 | 90.36 | 48.64 |
| AT3G27110 | 120.14 | 98.59 | 116.86 | 95.35 |
| AT3G23820 | 119.79 | 114.87 | 144.10 | 108.77 |
| AT1G74710 | 119.70 | 78.43 | 128.49 | 75.38 |
| AT2G37840 | 119.50 | 93.26 | 118.62 | 92.18 |
| AT5G48175 | 115.84 | 87.46 | 96.02 | 69.89 |
| AT3G09405 | 115.62 | 72.35 | 102.70 | 47.74 |
| AT1G07750 | 113.10 | 83.54 | 125.44 | 86.83 |
| AT5G09980 | 110.04 | 75.78 | 106.56 | 64.75 |
| AT3G53280 | 109.25 | 49.15 | 81.72 | 45.51 |
| AT3G01820 | 108.90 | 78.79 | 97.13 | 73.82 |
| AT2G44450 | 107.93 | 81.49 | 100.31 | 62.24 |
| AT3G44735 | 105.44 | 70.03 | 84.11 | 62.64 |
| AT1G53980 | 103.44 | 57.11 | 81.68 | 40.82 |
| AT3G17700 | 102.91 | 70.63 | 83.73 | 57.39 |
| AT2G16500 | 102.35 | 70.20 | 91.71 | 74.38 |
| AT5G10750 | 101.55 | 65.81 | 97.39 | 74.41 |
| AT5G60800 | 101.43 | 63.70 | 94.64 | 66.92 |
| AT1G10650 | 100.69 | 70.18 | 116.03 | 74.97 |
| AT1G53440 | 99.13 | 61.54 | 86.87 | 42.22 |
| AT1G16380 | 98.90 | 59.21 | 53.67 | 40.07 |
| AT3G04630 | 98.30 | 65.67 | 67.35 | 58.42 |
| AT2G40180 | 97.56 | 49.67 | 70.53 | 32.23 |
| AT5G25190 | 96.39 | 53.81 | 96.36 | 55.40 |
| AT2G45080 | 93.74 | 49.21 | 97.93 | 49.04 |
| AT3G08750 | 93.07 | 65.98 | 71.04 | 38.94 |
| AT5G63770 | 92.87 | 79.12 | 115.58 | 71.90 |
| AT3G49350 | 92.15 | 88.09 | 128.98 | 90.92 |
| AT4G09570 | 90.60 | 69.84 | 86.66 | 60.25 |
| AT2G20150 | 89.57 | 49.56 | 48.52 | 33.97 |
| AT4G37400 | 88.98 | 75.23 | 94.82 | 56.32 |
| AT2G04160 | 88.96 | 69.59 | 92.04 | 59.41 |
| AT5G52240 | 88.72 | 69.14 | 68.60 | 63.23 |
| AT1G24150 | 82.18 | 49.97 | 88.44 | 50.10 |
| AT3G03660 | 78.51 | 35.64 | 51.51 | 26.41 |
| AT1G05710 | 78.04 | 50.95 | 65.42 | 45.80 |
| AT1G28390 | 77.59 | 49.23 | 62.11 | 56.64 |
| AT4G02330 | 76.52 | 32.55 | 59.17 | 21.47 |
| AT5G41680 | 76.34 | 44.71 | 85.15 | 58.78 |
| AT3G48850 | 76.26 | 26.22 | 41.82 | 26.39 |
| AT1G05800 | 76.23 | 22.18 | 76.25 | 18.98 |
| AT1G53920 | 75.05 | 52.32 | 55.22 | 33.42 |
| AT2G32220 | 74.40 | 47.77 | 60.82 | 33.68 |
| AT4G39840 | 73.11 | 51.49 | 70.31 | 38.51 |
| AT2G37810 | 73.02 | 34.00 | 50.68 | 24.68 |
| AT2G22750 | 72.42 | 54.77 | 62.63 | 40.93 |
| AT2G01880 | 70.53 | 60.05 | 73.81 | 53.64 |
| AT4G19960 | 69.95 | 45.98 | 45.27 | 38.32 |
| AT4G11370 | 69.74 | 49.88 | 67.25 | 47.44 |
| AT1G05055 | 68.76 | 48.32 | 57.59 | 42.69 |
| AT4G15120 | 68.53 | 43.90 | 50.95 | 39.99 |
| AT1G52560 | 67.76 | 28.42 | 83.14 | 34.54 |
| AT4G30080 | 66.84 | 52.74 | 80.29 | 50.25 |
| AT1G29860 | 66.78 | 36.25 | 46.75 | 30.49 |
| AT4G14630 | 64.86 | 37.68 | 52.74 | 35.81 |
| AT5G38210 | 63.74 | 41.46 | 55.84 | 32.01 |
| AT5G66620 | 63.09 | 49.06 | 59.64 | 47.29 |
| AT4G38000 | 62.11 | 49.69 | 79.63 | 58.88 |
| AT5G65600 | 61.42 | 30.17 | 38.14 | 21.61 |
| AT5G07870 | 60.63 | 40.51 | 56.74 | 26.82 |
| AT2G24600 | 60.55 | 47.27 | 55.85 | 38.17 |
| AT2G26480 | 59.95 | 39.35 | 67.91 | 40.83 |
| AT2G38010 | 59.18 | 41.36 | 65.07 | 46.06 |
| AT5G58120 | 58.25 | 51.88 | 50.62 | 35.19 |
| AT1G21830 | 58.10 | 45.98 | 63.22 | 37.68 |
| AT1G77030 | 56.83 | 36.04 | 38.03 | 31.58 |
| AT1G63480 | 56.33 | 32.70 | 53.25 | 34.52 |
| AT4G28940 | 55.88 | 30.46 | 27.12 | 24.99 |
| AT2G46150 | 55.77 | 30.19 | 42.67 | 25.63 |
| AT5G41550 | 54.53 | 39.88 | 47.68 | 34.78 |
| AT3G49220 | 54.38 | 30.24 | 50.59 | 30.68 |
| AT4G17260 | 51.13 | 29.62 | 34.86 | 24.60 |
| AT3G09000 | 50.81 | 34.37 | 39.21 | 31.70 |
| AT3G27160 | 49.43 | 37.26 | 45.09 | 38.05 |
| AT4G11170 | 44.31 | 26.55 | 25.21 | 17.90 |
| AT1G44100 | 43.23 | 30.10 | 50.34 | 31.34 |
| AT5G56760 | 43.19 | 33.63 | 44.35 | 37.50 |
| AT4G34320 | 43.13 | 35.74 | 39.56 | 29.61 |
| AT1G17750 | 42.72 | 26.80 | 48.52 | 22.57 |
| AT1G70940 | 42.16 | 31.28 | 50.67 | 34.90 |
| AT2G35910 | 41.06 | 32.08 | 31.72 | 23.72 |
| AT1G59850 | 40.89 | 23.62 | 35.25 | 22.56 |
| AT5G62070 | 39.79 | 34.81 | 40.01 | 33.58 |
| AT3G50480 | 38.95 | 27.53 | 26.65 | 14.65 |
| AT1G53050 | 35.29 | 27.77 | 35.59 | 25.51 |
| AT5G13870 | 34.95 | 26.45 | 38.18 | 25.58 |
| AT1G63040 | 33.11 | 22.87 | 38.04 | 23.62 |
| AT5G67570 | 32.93 | 20.46 | 25.77 | 21.88 |
| AT1G58080 | 32.56 | 21.90 | 53.15 | 40.06 |
| AT1G73750 | 31.67 | 24.34 | 27.29 | 20.16 |
| AT4G02360 | 31.22 | 26.07 | 30.44 | 22.52 |
| AT3G10190 | 30.27 | 20.52 | 25.16 | 19.98 |
| AT4G26120 | 30.12 | 17.27 | 28.82 | 15.97 |
| AT5G58787 | 30.05 | 21.31 | 38.13 | 26.25 |
| AT4G36680 | 28.74 | 20.86 | 24.64 | 20.30 |
| AT5G22550 | 28.35 | 22.42 | 27.37 | 20.72 |
| AT1G67050 | 25.58 | 18.34 | 23.54 | 13.63 |
| AT3G60910 | 24.33 | 16.11 | 20.90 | 16.63 |
| AT3G05360 | 24.26 | 18.61 | 23.71 | 17.17 |
| AT1G57560 | 24.10 | 16.49 | 19.77 | 12.56 |
| AT2G34920 | 23.56 | 13.89 | 23.48 | 12.28 |
| AT3G20900 | 23.47 | 14.59 | 21.99 | 15.22 |
| AT4G39030 | 23.17 | 13.34 | 21.70 | 12.18 |
| AT1G68150 | 23.14 | 17.01 | 26.38 | 17.18 |
| AT1G51940 | 22.71 | 12.54 | 18.28 | 9.88 |
| AT4G40080 | 22.23 | 15.63 | 20.82 | 15.38 |
| AT1G18580 | 21.46 | 13.98 | 18.34 | 16.83 |
| AT5G07860 | 21.44 | 16.18 | 23.14 | 14.60 |
| AT1G32310 | 21.29 | 16.66 | 22.55 | 14.12 |
| AT5G24540 | 21.22 | 11.80 | 11.17 | 6.14 |
| AT1G74430 | 20.83 | 12.64 | 14.95 | 10.57 |
| AT5G52670 | 19.63 | 13.72 | 21.70 | 12.29 |
| AT1G44130 | 19.52 | 12.57 | 17.14 | 10.14 |
| AT1G24625 | 18.35 | 15.12 | 16.45 | 13.12 |
| AT1G19190 | 17.18 | 12.74 | 15.52 | 11.54 |
| AT5G44990 | 16.17 | 9.98 | 12.07 | 8.38 |
| AT3G63410 | 15.85 | 10.19 | 11.73 | 9.37 |
| AT1G60030 | 14.88 | 9.35 | 12.78 | 8.11 |
| AT3G54980 | 14.83 | 13.99 | 14.70 | 13.30 |
| AT1G35560 | 14.73 | 11.88 | 17.54 | 12.13 |
| AT2G41380 | 14.68 | 10.15 | 11.08 | 9.95 |
| AT5G38310 | 13.79 | 7.34 | 7.83 | 6.66 |
| AT1G15890 | 13.73 | 10.78 | 11.14 | 9.25 |
| AT1G09520 | 12.31 | 10.95 | 10.78 | 9.84 |
| AT1G56510 | 11.50 | 6.85 | 7.35 | 6.40 |
| AT1G36640 | 11.24 | 7.31 | 7.70 | 5.61 |
| AT1G35200 | 11.01 | 8.27 | 8.33 | 5.35 |
| AT5G40540 | 10.60 | 8.85 | 11.62 | 8.49 |
| AT4G27720 | 10.47 | 8.94 | 12.78 | 8.64 |
| AT4G33960 | 10.43 | 10.34 | 12.36 | 9.41 |
| AT2G46590 | 10.15 | 7.44 | 9.91 | 6.51 |
| AT2G21560 | 10.04 | 8.09 | 14.38 | 9.82 |
| AT1G14480 | 9.06 | 5.96 | 7.07 | 5.94 |
| AT3G50760 | 8.95 | 7.09 | 8.54 | 7.09 |
| AT2G17040 | 8.67 | 5.06 | 8.13 | 4.96 |
| AT2G19130 | 8.62 | 6.93 | 7.97 | 7.00 |
| AT1G11000 | 8.36 | 6.90 | 8.58 | 5.95 |
| AT2G16870 | 7.87 | 6.66 | 6.93 | 5.96 |
| AT3G61900 | 6.57 | 6.11 | 7.57 | 6.19 |
| AT4G23440 | 5.43 | 5.36 | 6.00 | 5.54 |
| AT4G30560 | 5.33 | 4.99 | 4.92 | 4.92 |
| AT5G39710 | 5.21 | 4.99 | 4.98 | 4.98 |
| AT2G39900 | 5.15 | 4.95 | 4.98 | 4.95 |
| AT1G55610 | 5.00 | 4.96 | 5.43 | 4.98 |

TABLE 16
Significantly over-represented GO terms (FDR < 0.01) identified for genes up-
regulated or down-regulated by DEX-induced nuclear import of bZIP1 (FDR < 0.05).

| | | |
|---|---|---|
| Term | p-value | Genes |

A. Significantly over-represented GO terms in the DEX up-regulated genes

| | | | |
|---|---|---|---|
| GO: 0009310 | amine catabolic | 0.000255 | AT4G33150\|AT3G30775\|AT2G43400\|AT1G08630\|AT5G43430\|AT1G64660\| |
| | process | | AT1G03090\|AT1G65840\|AT5G54080 |
| GO: 0042221 | response to | 0.000255 | AT1G08720\|AT1G08920\|AT5G66400\|AT2G40170\|AT2G22080\|AT4G13430\| |
| | chemical | | AT4G37790\|AT2G34600\|AT1G54100\|AT5G37260\|AT3G51860\| |
| | stimulus | | AT5G61590\|AT5G47390\|AT5G16970\|AT2G38750\|AT4G37220\|AT5G16960\| |
| | | | AT1G04410\|AT1G49670\|AT3G11410\|AT4G32320\|AT5G67450\| |
| | | | AT5G07440\|AT1G08090\|AT5G54500\|AT5G50200\|AT2G23170\|AT1G08830\| |
| | | | AT3G56240\|AT1G55020\|AT4G33420\|AT1G20340\|AT4G27260\| |
| | | | AT5G59220\|AT1G28130\|AT2G19810\|AT3G05200\|AT2G46270\|AT5G03720\| |
| | | | AT3G23230\|AT5G01600\|AT1G73260\|AT1G08930\|AT5G39040\| |
| | | | AT5G44380\|AT1G18330\|AT5G13740\|AT4G30170\|AT4G35770\|AT1G16150\| |
| | | | AT1G15050\|AT2G14170\|AT1G80460\|AT5G10450\|AT1G43160\| |
| | | | AT4G39070\|AT5G67300\|AT3G14050\|AT3G14990\|AT4G21440\|AT1G02860\| |
| | | | AT3G30775\|AT5G18170\|AT1G68850\|AT4G34350\|AT2G01570\| |
| | | | AT3G60690\|AT5G05340\|AT1G17190 |
| GO: 0050896 | response to | 0.000255 | AT1G08920\|AT2G43400\|AT2G33150\|AT5G02810\|AT2G40170\|AT2G22080\| |
| | stimulus | | AT4G13430\|AT4G37790\|AT1G54100\|AT1G02670\|AT5G61590\| |
| | | | AT5G47390\|AT3G54960\|AT2G38750\|AT4G37220\|AT5G16960\|AT1G04410\| |
| | | | AT1G49670\|AT3G11410\|AT4G32320\|AT5G07440\|AT1G08090\| |
| | | | AT5G54500\|AT1G08830\|AT1G25275\|AT3G15950\|AT4G33420\|AT4G27260\| |
| | | | AT5G59220\|AT1G28130\|AT5G24470\|AT2G46270\|AT5G03720\| |
| | | | AT3G23230\|AT1G06520\|AT5G67320\|AT1G73260\|AT5G39040\|AT5G40780\| |
| | | | AT4G30170\|AT4G35770\|AT1G16150\|AT1G31480\|AT1G80460\| |
| | | | AT5G24530\|AT1G75800\|AT1G43160\|AT2G39980\|AT4G39070\|AT3G14050\| |
| | | | AT3G14990\|AT1G60940\|AT3G15620\|AT5G06980\|AT1G02860\| |
| | | | AT3G47640\|AT3G30775\|AT1G68850\|AT2G26280\|AT5G13750\|AT3G45060\| |
| | | | AT1G17190\|AT5G67440\|AT5G27350\|AT1G08720\|AT5G20150\| |
| | | | AT5G66400\|AT5G47740\|AT5G52250\|AT4G24220\|AT2G34600\|AT5G37260\| |
| | | | AT3G51860\|AT5G16970\|AT3G61060\|AT3G27690\|AT5G67450\| |
| | | | AT5G47240\|AT5G50200\|AT2G23170\|AT4G01120\|AT5G61510\|AT3G56240\| |
| | | | AT1G55020\|AT1G20340\|AT5G43580\|AT5G04770\|AT2G39200\| |
| | | | AT2G19810\|AT3G05200\|AT5G01600\|AT1G08930\|AT4G37590\|AT5G44380\| |
| | | | AT1G18330\|AT5G13740\|AT4G36040\|AT1G15050\|AT2G14170\| |
| | | | AT1G13080\|AT5G64120\|AT5G10450\|AT5G20250\|AT5G67300\|AT2G32660\| |
| | | | AT4G21440\|AT1G75230\|AT5G18170\|AT4G34350\|AT2G01570\| |
| | | | AT3G60690\|AT5G05340\|AT5G61600 |
| GO: 0016054 | organic acid | 0.000434 | AT3G30775\|AT2G43400\|AT2G33150\|AT5G43430\|AT1G64660\|AT4G33150\| |
| | catabolic | | AT3G51840\|AT1G08630\|AT5G65110\|AT1G03090\|AT5G54080 |
| | process | | |
| GO: 0046395 | carboxylic acid | 0.000434 | AT3G30775\|AT2G43400\|AT2G33150\|AT5G43430\|AT1G64660\|AT4G33150\| |
| | catabolic | | AT3G51840\|AT1G08630\|AT5G65110\|AT1G03090\|AT5G54080 |
| | process | | |
| GO: 0009063 | cellular amino | 0.000585 | AT4G33150\|AT3G30775\|AT2G43400\|AT1G08630\|AT5G43430\|AT1G64660\| |
| | acid catabolic | | AT1G03090\|AT5G54080 |
| | process | | |
| GO: 0009628 | response to | 0.00178 | AT1G08720\|AT1G08920\|AT2G43400\|AT5G02810\|AT5G66400\|AT5G52250\| |
| | abiotic stimulus | | AT1G54100\|AT5G37260\|AT5G61590\|AT5G47390\|AT2G38750\| |
| | | | AT1G04410\|AT3G11410\|AT3G27690\|AT5G67450\|AT5G07440\|AT4G01120\| |
| | | | AT5G61510\|AT1G08830\|AT1G25275\|AT3G56240\|AT3G15950\| |
| | | | AT1G20340\|AT5G59220\|AT5G24470\|AT5G03720\|AT1G06520\|AT5G67320\| |
| | | | AT5G01600\|AT1G73260\|AT1G08930\|AT5G40780\|AT4G37590\| |
| | | | AT1G18330\|AT1G31480\|AT1G80460\|AT1G13080\|AT5G20250\|AT1G43160\| |
| | | | AT2G39980\|AT4G39070\|AT5G67300\|AT1G60940\|AT3G15620\| |
| | | | AT5G06980\|AT4G21440\|AT5G18170\|AT2G01570\|AT5G13750\|AT3G45060\| |
| | | | AT1G17190\|AT5G67440 |
| GO: 0006950 | response to | 0.00375 | AT1G08920\|AT2G33150\|AT2G22080\|AT1G54100\|AT1G02670\|AT5G61590\| |
| | stress | | AT5G47390\|AT3G54960\|AT2G38750\|AT4G37220\|AT5G16960\| |
| | | | AT1G04410\|AT1G49670\|AT3G11410\|AT4G32320\|AT5G07440\|AT1G08830\| |
| | | | AT3G15950\|AT4G33420\|AT5G59220\|AT5G03720\|AT5G67320\| |
| | | | AT1G73260\|AT4G30170\|AT4G35770\|AT1G43160\|AT3G14050\|AT1G60940\| |
| | | | AT1G02860\|AT3G47640\|AT3G30775\|AT1G68850\|AT2G26280\| |
| | | | AT1G17190\|AT1G08720\|AT5G20150\|AT5G66400\|AT5G47740\|AT4G24220\| |
| | | | AT5G37260\|AT5G16970\|AT3G61060\|AT5G67450\|AT5G47240\| |
| | | | AT5G50200\|AT3G56240\|AT1G55020\|AT5G43580\|AT2G39200\|AT2G19810\| |
| | | | AT5G01600\|AT1G08930\|AT5G44380\|AT1G18330\|AT4G36040\| |
| | | | AT2G14170\|AT1G13080\|AT5G64120\|AT5G10450\|AT5G20250\|AT5G67300\| |
| | | | AT2G32660\|AT4G21440\|AT1G75230\|AT5G18170\|AT2G01570\| |
| | | | AT5G05340\|AT5G61600 |
| GO: 0006979 | response to | 0.00375 | AT2G19810\|AT2G22080\|AT5G01600\|AT1G73260\|AT1G08830\|AT3G56240\| |
| | oxidative stress | | AT5G16970\|AT3G30775\|AT1G68850\|AT4G33420\|AT5G44380\| |
| | | | AT4G30170\|AT5G16960\|AT4G35770\|AT5G05340\|AT2G14170\|AT1G49670\| |
| | | | AT4G32320 |
| GO: 0009081 | branched chain | 0.0044 | AT1G18270\|AT1G10070\|AT5G43430\|AT1G10060\|AT1G03090\|AT2G43400 |
| | family amino | | |
| | acid metabolic | | |
| | process | | |
| GO: 0044282 | small molecule | 0.00497 | AT3G30775\|AT2G43400\|AT2G33150\|AT5G43430\|AT3G51840\|AT4G33150\| |
| | catabolic | | AT5G65110\|AT1G03090\|AT1G18270\|AT1G64660\|AT1G80460\| |
| | process | | AT1G08630\|AT5G54080 |
| GO: 0048878 | chemical | 0.00601 | AT3G47640\|AT1G20340\|AT5G24030\|AT5G13740\|AT2G23170\|AT4G27260\| |
| | homeostasis | | AT5G47560\|AT3G51860\|AT1G28130\|AT5G01600\|AT3G56240 |

B. Significantly over-represented GO terms in the DEX down-regulated genes

| | | | |
|---|---|---|---|
| GO: 0050896 | response to | 4.68E−09 | AT4G23440\|AT3G52360\|AT4G17230\|AT4G16780\|AT5G24620\|AT2G35980\| |
| | stimulus | | AT1G80820\|AT4G17260\|AT2G46140\|AT4G34180\|AT3G11840\| |
| | | | AT5G62390\|AT3G02880\|AT3G24550\|AT1G61560\|AT1G18890\|AT4G02200\| |
| | | | AT4G30080\|AT5G44070\|AT3G61850\|AT5G01540\|AT1G11210\| |
| | | | AT4G12720\|AT1G09940\|AT2G01150\|AT5G51190\|AT1G13340\|AT3G44720\| |
| | | | AT2G17040\|AT4G39260\|AT1G55920\|AT1G20510\|AT3G61900\| |
| | | | AT4G33300\|AT3G45640\|AT2G38870\|AT3G25070\|AT1G57630\|AT1G07520\| |
| | | | AT3G06490\|AT2G34930\|AT3G17020\|AT3G50480\|AT5G62680\| |
| | | | AT1G80530\|AT5G61210\|AT5G44610\|AT5G66070\|AT2G26560\|AT3G07390\| |
| | | | AT1G73010\|AT2G40180\|AT4G11360\|AT1G56510\|AT5G63770\| |
| | | | AT4G11170\|AT2G41380\|AT5G25190\|AT5G65020\|AT3G13650\|AT2G06050\| |
| | | | AT3G52430\|AT4G37910\|AT1G11000\|AT5G06720\|AT5G66880\| |
| | | | AT3G59900\|AT5G48540\|AT1G18570\|AT2G04160\|AT3G05360\|AT2G39660\| |
| | | | AT1G72060\|AT5G37770\|AT1G11310\|AT1G15890\|AT3G48090\| |
| | | | AT5G04720\|AT4G26120\|AT4G34150\|AT4G39030\|AT1G52560\|AT1G05710\| |
| | | | AT5G24540\|AT5G22690\|AT3G52400\|AT2G17660\|AT1G05055\| |
| | | | AT3G28740\|AT4G02380\|AT2G19190\|AT1G52200\|AT1G17750\|AT1G74430\| |
| | | | AT1G05800\|AT1G66090\|AT3G17700\|AT1G30040\|AT4G14630\| |
| | | | AT1G14550\|AT5G26030\|AT4G11850\|AT5G09980\|AT5G41550\|AT5G58120\| |
| | | | AT3G28580\|AT2G38470\|AT1G19220\|AT4G18880\|AT3G11820\| |
| | | | AT2G26380\|AT1G74710\|AT2G16870\|AT2G16500\|AT1G57560\|AT1G70940\| |
| | | | AT5G47910\|AT1G02400\|AT5G54170\|AT2G46590\|AT1G14540\| |
| | | | AT3G09270\|AT5G49620 |
| GO: 0006952 | defense | 3.03E−08 | AT2G38870\|AT3G52430\|AT3G25070\|AT4G11850\|AT4G23440\|AT1G11000\| |
| | response | | AT1G57630\|AT2G35980\|AT1G18570\|AT5G41550\|AT5G58120\| |
| | | | AT2G38470\|AT2G34930\|AT3G05360\|AT2G39660\|AT5G37770\|AT3G11840\| |
| | | | AT1G11310\|AT3G11820\|AT2G26380\|AT1G74710\|AT1G61560\| |
| | | | AT2G26560\|AT1G15890\|AT3G48090\|AT5G04720\|AT2G16870\|AT4G39030\| |
| | | | AT5G44070\|AT5G47910\|AT1G56510\|AT4G12720\|AT5G22690\| |
| | | | AT4G11170\|AT3G52400\|AT3G28740\|AT2G19190\|AT1G17750\|AT4G39260\| |
| | | | AT1G05800\|AT3G13650\|AT1G66090\|AT4G33300 |
| GO: 0006950 | response to | 9.90E−08 | AT4G23440\|AT2G35980\|AT1G80820\|AT4G17260\|AT2G46140\|AT4G34180\| |
| | stress | | AT3G11840\|AT5G62390\|AT3G24550\|AT1G61560\|AT4G02200\| |
| | | | AT5G44070\|AT1G11210\|AT4G12720\|AT1G09940\|AT1G13340\|AT4G39260\| |
| | | | AT1G55920\|AT1G20510\|AT4G33300\|AT3G45640\|AT2G38870\| |
| | | | AT3G25070\|AT1G57630\|AT3G06490\|AT2G34930\|AT3G17020\|AT5G44610\| |
| | | | AT2G26560\|AT1G73010\|AT1G56510\|AT5G63770\|AT4G11170\| |
| | | | AT5G65020\|AT3G13650\|AT2G06050\|AT3G52430\|AT4G37910\|AT1G11000\| |
| | | | AT5G66880\|AT5G06720\|AT1G18570\|AT3G05360\|AT2G39660\| |
| | | | AT1G72060\|AT5G37770\|AT1G11310\|AT1G15890\|AT3G48090\|AT5G04720\| |
| | | | AT4G34150\|AT4G39030\|AT1G52560\|AT5G22690\|AT3G52400\| |
| | | | AT1G05055\|AT3G28740\|AT4G02380\|AT2G19190\|AT1G52200\|AT1G17750\| |
| | | | AT1G05800\|AT1G66090\|AT4G14630\|AT1G14550\|AT5G26030\| |
| | | | AT4G11850\|AT5G41550\|AT5G58120\|AT2G38470\|AT3G11820\|AT2G26380\| |
| | | | AT1G74710\|AT2G16870\|AT2G16500\|AT5G47910\|AT5G54170\| |
| | | | AT2G46590\|AT1G14540\|AT5G49620 |
| GO: 0051707 | response to | 1.21E−06 | AT3G45640\|AT2G06050\|AT2G38870\|AT3G52430\|AT3G25070\|AT4G11850\| |
| | other | | AT5G24620\|AT2G35980\|AT1G18570\|AT2G38470\|AT3G06490\| |
| | organism | | AT2G34930\|AT3G50480\|AT2G39660\|AT5G61210\|AT1G11310\|AT3G11820\| |
| | | | AT1G74710\|AT3G24550\|AT1G61560\|AT2G26560\|AT3G48090\| |
| | | | AT4G39030\|AT5G44070\|AT5G47910\|AT1G56510\|AT4G12720\|AT5G24540\| |
| | | | AT3G52400\|AT3G28740\|AT2G19190\|AT1G17750\|AT1G05800\| |
| | | | AT3G17700 |
| GO: 0009607 | response to | 2.35E−06 | AT3G45640\|AT2G06050\|AT2G38870\|AT3G52430\|AT3G25070\|AT4G11850\| |
| | biotic | | AT5G24620\|AT2G35980\|AT1G18570\|AT2G38470\|AT3G06490\| |
| | stimulus | | AT2G34930\|AT3G50480\|AT2G39660\|AT5G61210\|AT5G62390\|AT1G11310\| |
| | | | AT3G11820\|AT1G74710\|AT3G24550\|AT1G61560\|AT2G26560\| |
| | | | AT3G48090\|AT4G39030\|AT5G44070\|AT5G47910\|AT1G56510\|AT4G12720\| |
| | | | AT5G24540\|AT3G52400\|AT3G28740\|AT2G19190\|AT1G17750\| |
| | | | AT1G05800\|AT3G17700 |
| GO: 0051704 | multi- | 2.77E−06 | AT3G45640\|AT2G06050\|AT2G38870\|AT3G52430\|AT3G25070\|AT4G11850\| |
| | organism | | AT5G24620\|AT2G35980\|AT1G18570\|AT2G38470\|AT3G06490\| |
| | process | | AT2G34930\|AT3G50480\|AT2G39660\|AT5G61210\|AT1G11310\|AT3G11820\| |
| | | | AT1G74710\|AT3G24550\|AT1G61560\|AT2G26560\|AT3G48090\| |
| | | | AT4G39030\|AT5G44070\|AT5G47910\|AT1G56510\|AT4G12720\|AT5G24540\| |
| | | | AT3G52400\|AT3G28740\|AT2G19190\|AT1G17750\|AT1G05800\| |
| | | | AT3G17700 |
| GO: 0002376 | immune | 1.12E−05 | AT3G48090\|AT3G52430\|AT2G16870\|AT3G25070\|AT4G11850\|AT4G23440\| |
| | system | | AT1G57630\|AT1G56510\|AT2G35980\|AT4G12720\|AT5G41550\| |
| | process | | AT5G58120\|AT5G22690\|AT3G05360\|AT5G37770\|AT3G11840\|AT4G39260\| |
| | | | AT1G11310\|AT1G74710\|AT1G66090\|AT1G61560\|AT2G26560 |
| GO: 0042221 | response to | 1.18E−05 | AT2G06050\|AT3G52430\|AT4G37910\|AT4G17230\|AT5G06720\|AT5G66880\| |
| | chemical | | AT3G59900\|AT4G16780\|AT1G18570\|AT2G04160\|AT4G17260\| |
| | stimulus | | AT2G46140\|AT1G72060\|AT5G37770\|AT3G11840\|AT5G62390\|AT3G02880\| |
| | | | AT3G48090\|AT4G26120\|AT1G18890\|AT4G02200\|AT4G30080\| |
| | | | AT5G44070\|AT5G01540\|AT1G52560\|AT1G11210\|AT1G05710\|AT4G12720\| |
| | | | AT1G09940\|AT5G51190\|AT3G52400\|AT1G13340\|AT2G17660\| |
| | | | AT4G02380\|AT1G52200\|AT2G17040\|AT1G17750\|AT4G39260\|AT1G74430\| |
| | | | AT3G61900\|AT3G45640\|AT1G14550\|AT3G25070\|AT5G26030\| |
| | | | AT1G07520\|AT5G09980\|AT3G28580\|AT2G38470\|AT3G06490\|AT1G19220\| |
| | | | AT4G18880\|AT5G61210\|AT5G44610\|AT3G11820\|AT5G66070\| |
| | | | AT2G26560\|AT3G07390\|AT2G16500\|AT1G57560\|AT2G40180\|AT4G11360\| |
| | | | AT4G11170\|AT2G41380\|AT5G25190\|AT1G14540\|AT5G65020\| |
| | | | AT3G09270\|AT5G49620 |
| GO: 0031348 | negative | 3.00E−05 | AT3G25070\|AT1G11310\|AT3G52400\|AT3G11820\|AT4G39030\|AT1G74710\| |
| | regulation of | | AT3G52430 |
| | defense | | |
| | response | | |
| GO: 0045087 | innate | 6.55E−05 | AT3G48090\|AT3G52430\|AT2G16870\|AT3G25070\|AT4G11850\|AT4G23440\| |
| | immune | | AT1G57630\|AT1G56510\|AT4G12720\|AT5G41550\|AT5G58120\| |
| | response | | AT5G22690\|AT5G37770\|AT4G39260\|AT1G11310\|AT1G74710\|AT1G66090\| |
| | | | AT1G61560\|AT2G26560 |
| GO: 0006955 | immune | 7.49E−05 | AT3G48090\|AT3G52430\|AT2G16870\|AT3G25070\|AT4G11850\|AT4G23440\| |
| | response | | AT1G57630\|AT1G56510\|AT4G12720\|AT5G41550\|AT5G58120\| |
| | | | AT5G22690\|AT5G37770\|AT4G39260\|AT1G11310\|AT1G74710\|AT1G66090\| |
| | | | AT1G61560\|AT2G26560 |
| GO: 0009620 | response to | 0.000103 | AT2G06050\|AT2G38470\|AT3G06490\|AT2G34930\|AT2G38870\|AT3G52400\| |
| | fungus | | AT2G39660\|AT5G47910\|AT1G56510\|AT1G11310\|AT3G11820\| |
| | | | AT1G05800\|AT1G74710\|AT3G24550\|AT1G61560 |
| GO: 0080134 | regulation of | 0.000169 | AT3G45640\|AT1G11310\|AT3G11820\|AT2G31880\|AT3G52430\|AT4G12720\| |
| | response to | | AT3G25070\|AT3G52400\|AT4G39030\|AT1G74710\|AT3G05360 |
| | stress | | |
| GO: 0016310 | phosphorylation | 0.00018 | AT3G45640\|AT5G40540\|AT3G25070\|AT1G55610\|AT5G41680\|AT1G16670\| |
| | | | AT2G41890\|AT2G17220\|AT1G51940\|AT4G09570\|AT2G31880\| |
| | | | AT4G28350\|AT2G19130\|AT5G38210\|AT1G70130\|AT3G55950\|AT2G37840\| |
| | | | AT3G16030\|AT1G51620\|AT2G39660\|AT1G70530\|AT3G02880\| |
| | | | AT1G53430\|AT1G61370\|AT3G24550\|AT3G08760\|AT2G11520\|AT1G18890\| |
| | | | AT4G21390\|AT5G07620\|AT1G53440\|AT1G28390\|AT5G65600\| |
| | | | AT1G04440\|AT2G39110\|AT1G17750\|AT4G08850\|AT1G53050\|AT4G39940 |
| GO: 0031347 | regulation of | 0.000214 | AT1G11310\|AT3G11820\|AT2G31880\|AT3G52430\|AT4G12720\|AT3G25070\| |
| | defense | | AT3G52400\|AT4G39030\|AT1G74710\|AT3G05360 |
| | response | | |
| GO: 0010033 | response to | 0.000224 | AT3G52430\|AT4G17230\|AT5G66880\|AT3G59900\|AT4G16780\|AT1G18570\| |
| | organic | | AT2G04160\|AT4G17260\|AT5G37770\|AT3G11840\|AT5G62390\| |
| | substance | | AT3G02880\|AT3G48090\|AT4G26120\|AT1G18890\|AT4G30080\|AT5G01540\| |
| | | | AT1G05710\|AT5G51190\|AT3G52400\|AT2G17040\|AT1G17750\| |
| | | | AT4G39260\|AT1G74430\|AT3G61900\|AT3G45640\|AT3G25070\|AT1G07520\| |
| | | | AT5G09980\|AT3G28580\|AT2G38470\|AT3G06490\|AT1G19220\| |
| | | | AT4G18880\|AT5G61210\|AT5G44610\|AT3G11820\|AT5G66070\|AT3G07390\| |
| | | | AT1G57560\|AT2G40180\|AT4G11360\|AT5G25190\|AT5G49620 |
| GO: 0006468 | protein | 0.000235 | AT3G45640\|AT5G40540\|AT3G25070\|AT1G55610\|AT5G41680\|AT1G16670\| |
| | phosphorylation | | AT2G41890\|AT2G17220\|AT1G51940\|AT4G09570\|AT2G31880\| |
| | | | AT4G28350\|AT2G19130\|AT5G38210\|AT1G70130\|AT3G55950\|AT2G37840\| |
| | | | AT3G16030\|AT1G51620\|AT2G39660\|AT1G70530\|AT3G02880\| |
| | | | AT1G53430\|AT1G61370\|AT3G24550\|AT3G08760\|AT2G11520\|AT1G18890\| |
| | | | AT4G21390\|AT5G07620\|AT1G53440\|AT1G28390\|AT5G65600\| |
| | | | AT1G04440\|AT2G39110\|AT1G17750\|AT4G08850\|AT1G53050 |
| GO: 0006793 | phosphorus | 0.000373 | AT3G45640\|AT5G40540\|AT3G25070\|AT1G55610\|AT5G41680\|AT1G16670\| |
| | metabolic | | AT2G41890\|AT2G17220\|AT1G51940\|AT4G09570\|AT2G31880\| |
| | process | | AT4G28350\|AT2G19130\|AT5G38210\|AT1G70130\|AT3G55950\|AT2G37840\| |
| | | | AT3G16030\|AT1G51620\|AT2G39660\|AT1G70530\|AT3G02880\| |
| | | | AT1G53430\|AT1G61370\|AT3G24550\|AT3G08760\|AT2G11520\|AT1G18890\| |
| | | | AT4G21390\|AT5G07620\|AT1G53440\|AT1G28390\|AT5G65600\| |
| | | | AT1G04440\|AT3G02800\|AT2G39110\|AT1G17750\|AT4G08850\|AT1G53050\| |
| | | | AT4G39940 |
| GO: 0006796 | phosphate | 0.000373 | AT3G45640\|AT5G40540\|AT3G25070\|AT1G55610\|AT5G41680\|AT1G16670\| |
| | metabolic | | AT2G41890\|AT2G17220\|AT1G51940\|AT4G09570\|AT2G31880\| |
| | process | | AT4G28350\|AT2G19130\|AT5G38210\|AT1G70130\|AT3G55950\|AT2G37840\| |
| | | | AT3G16030\|AT1G51620\|AT2G39660\|AT1G70530\|AT3G02880\| |
| | | | AT1G53430\|AT1G61370\|AT3G24550\|AT3G08760\|AT2G11520\|AT1G18890\| |
| | | | AT4G21390\|AT5G07620\|AT1G53440\|AT1G28390\|AT5G65600\| |
| | | | AT1G04440\|AT3G02800\|AT2G39110\|AT1G17750\|AT4G08850\|AT1G53050\| |
| | | | AT4G39940 |
| GO: 0050832 | defense | 0.00054 | AT2G38470\|AT2G34930\|AT2G38870\|AT3G52400\|AT2G39660\|AT5G47910\| |
| | response to | | AT1G56510\|AT1G11310\|AT3G11820\|AT1G05800\|AT1G74710\| |
| | fungus | | AT1G61560 |
| GO: 0008219 | cell death | 0.000593 | AT5G22690\|AT3G48090\|AT5G04720\|AT2G16870\|AT3G25070\|AT4G23440\| |
| | | | AT1G11000\|AT1G11310\|AT4G12720\|AT1G66090\|AT5G41550\| |
| | | | AT1G61560\|AT5G58120\|AT4G33300\|AT2G26560\|AT1G15890 |
| GO: 0016265 | death | 0.000593 | AT5G22690\|AT3G48090\|AT5G04720\|AT2G16870\|AT3G25070\|AT4G23440\| |
| | | | AT1G11000\|AT1G11310\|AT4G12720\|AT1G66090\|AT5G41550\| |
| | | | AT1G61560\|AT5G58120\|AT4G33300\|AT2G26560\|AT1G15890 |
| GO: 0010200 | response to | 0.00127 | AT3G45640\|AT2G17040\|AT3G11840\|AT1G07520\|AT2G38470\|AT4G18880\| |
| | chitin | | AT5G51190\|AT4G26120\|AT5G66070\|AT4G17230\|AT4G11360 |
| GO: 0048583 | regulation of | 0.00199 | AT3G45640\|AT3G52430\|AT3G25070\|AT3G52400\|AT4G39030\|AT3G05360\| |
| | response to | | AT5G66880\|AT4G09570\|AT1G11310\|AT3G11820\|AT2G31880\| |
| | stimulus | | AT4G12720\|AT1G74710 |
| GO: 0012501 | programmed | 0.00424 | AT5G22690\|AT3G48090\|AT5G04720\|AT2G16870\|AT3G25070\|AT4G23440\| |
| | cell death | | AT4G12720\|AT1G66090\|AT5G41550\|AT5G58120\|AT4G33300\| |
| | | | AT2G26560\|AT1G15890 |
| GO: 0006979 | response to | 0.0049 | AT3G45640\|AT3G48090\|AT1G14550\|AT5G26030\|AT2G16500\|AT5G06720\| |
| | oxidative | | AT1G52560\|AT1G11210\|AT4G12720\|AT1G09940\|AT1G13340\| |
| | stress | | AT4G02380\|AT1G14540\|AT1G52200\|AT1G72060\|AT5G37770 |
| GO: 0006464 | protein | 0.0081 | AT5G40540\|AT1G55610\|AT2G41890\|AT2G17220\|AT2G19130\|AT5G38210\| |
| | modification | | AT2G39660\|AT3G11840\|AT3G02880\|AT1G53430\|AT3G24550\| |
| | process | | AT2G11520\|AT1G18890\|AT5G57500\|AT4G12720\|AT5G65600\|AT1G04440\| |
| | | | AT1G17750\|AT4G08850\|AT3G45640\|AT3G25070\|AT5G41680\| |
| | | | AT1G16670\|AT3G61390\|AT1G51940\|AT4G09570\|AT2G31880\|AT4G28350\| |
| | | | AT1G70130\|AT3G55950\|AT2G37840\|AT3G16030\|AT1G51620\| |
| | | | AT1G70530\|AT1G61370\|AT3G08760\|AT4G21390\|AT5G07620\|AT1G53440\| |
| | | | AT1G28390\|AT2G38830\|AT2G39110\|AT1G53050 |

TABLE 17
Genes regulated by DEX-induced nuclear import of bZIP1 (FDR <0.05)
and by the interaction of N-signal and DEX-induced nuclear import
of bZIP1 (p-val <0.01).

| | |
|---|---|
| Cluster1 | |
| At4g37540 | LBD39, LOB domain-containing protein 39 |
| At5g04630 | CYP77A9, cytochrome P450, family 77, subfamily A, |
| | polypeptide 9 |
| At3g60690 | SAUR-like auxin-responsive protein family |
| At4g38340 | NLP3; Plant regulator RWP-RK family protein |
| Cluster2 | |
| At4g33420 | Peroxidase superfamily protein |
| At2g31380 | STH, salt tolerance homologue |
| At3g30396 | transposable element gene |
| At1g15050 | IAA34, indole-3-acetic acid inducible 34 |
| At5g28050 | Cytidine/deoxycytidylate deaminase family protein |
| At1g01490 | Heavy metal transport/detoxification superfamily protein |
| At2g39570 | ACT domain-containing protein |
| At3g55150 | ATEXO70H1, EXO70H1, exocyst subunit exo70 |
| | family protein H1 |
| At2g28630 | KCS12, 3-ketoacyl-CoA synthase 12 |
| At2g02700 | Cysteine/Histidine-rich C1 domain family protein |
| Cluster3 | |
| At1g55610 | BRL1, BRI1 like |
| At4g33960 | unknown protein; |
| At3g23820 | GAE6, UDP-D-glucuronate 4-epimerase 6 |
| At3g49350 | Ypt/Rab-GAP domain of gyp1p superfamily protein |
| Cluster4 | |
| At1g56510 | ADR2, WRR4, Disease resistance protein |
| | (TIR-NBS-LRR class) |
| At3g14360 | alpha/beta-Hydrolases superfamily protein |
| At3g59900 | ARGOS, auxin-regulated gene involved in organ size |
| At4g30560 | ATCNGC9, CNGC9, cyclic nucleotide gated channel 9 |
| At5g61210 | ATSNAP33, ATSNAP33B, SNAP33, SNP33, soluble |
| | N-ethylmaleimide-sensitive factor adaptor protein 33 |
| At5g39710 | EMB2745, Tetratricopeptide repeat (TPR)-like |
| | superfamily protein |
| At3g63390 | unknown protein; |
| At4g28940 | Phosphorylase superfamily protein |
| At2g39900 | GATA type zinc finger transcription factor family protein |
| At3g53280 | CYP71B5, cytochrome p450 71b5 |

TABLE 18
Genes bound by GR::bZIP1 as detected by ChIP-seq with anti-GR antibody.

| | present in | | |
|---|---|---|---|
| | ATH1 | | |
| | microarray | | |
| | by | | |
| bZIP1 bound | unambiguous | | |
| genes | probes | | |
| AT1G01060 | YES | LHY | LATE ELONGATED HYPOCOTYL |
| AT1G01460 | YES | ATPIPK11 | |
| AT1G01470 | YES | LEA14 | LATE EMBRYOGENESIS ABUNDANT 14 |
| AT1G01550 | YES | BPS1 | BYPASS 1 |
| AT1G01560 | YES | ATMPK11 | MAP kinase 11 |
| AT1G01720 | YES | ANAC2 | Arabidopsis NAC domain containing protein 2 |
| AT1G01725 | YES | | |
| AT1G03850 | YES | ATGRXS13 | glutaredoxin 13 |
| AT1G04530 | YES | TPR4 | tetratricopeptide repeat 4 |
| AT1G05330 | YES | | |
| AT1G05340 | YES | | |
| AT1G05680 | YES | UGT74E2 | Uridine diphosphate glycosyltransferase 74E2 |
| AT1G06760 | YES | | |
| AT1G08510 | YES | FATB | fatty acyl-ACP thioesterases B |
| AT1G08940 | YES | | |
| AT1G09070 | YES | (AT)SRC2 | SOYBEAN GENE REGULATED BY COLD-2 |
| AT1G09080 | YES | BIP3 | binding protein 3 |
| AT1G09930 | YES | ATOPT2 | oligopeptide transporter 2 |
| AT1G10170 | YES | ATNFXL1 | NF-X-like 1 |
| AT1G11560 | YES | | |
| AT1G11670 | YES | | |
| AT1G12960 | YES | | |
| AT1G13210 | YES | ACA.1 | autoinhibited Ca2+/ATPase II |
| AT1G13260 | YES | EDF4 | ETHYLENE RESPONSE DNA BINDING FACTOR 4 |
| AT1G13270 | YES | MAP1B | METHIONINE AMINOPEPTIDASE 1B |
| AT1G14040 | YES | | |
| AT1G14530 | YES | THH1 | TOM THREE HOMOLOG 1 |
| AT1G14540 | YES | PER4 | peroxidase 4 |
| AT1G14550 | YES | | |
| AT1G14560 | YES | | |
| AT1G15010 | YES | | |
| AT1G15040 | YES | GAT | glutamine amidotransferase |
| AT1G15080 | YES | ATLPP2 | LIPID PHOSPHATE PHOSPHATASE 2 |
| AT1G16640 | YES | | |
| AT1G16670 | YES | | |
| AT1G17180 | YES | ATGSTU25 | glutathione S-transferase TAU 25 |
| AT1G17420 | YES | ATLOX3 | Arabidopsis thaliana lipoxygenase 3 |
| AT1G17850 | YES | | |
| AT1G17860 | YES | | |
| AT1G17870 | YES | ATEGY3 | ETHYLENE-DEPENDENT GRAVITROPISM-DEFICIENT |
| | | | AND YELLOW-GREEN-LIKE 3 |
| AT1G18210 | YES | | |
| AT1G18310 | YES | | |
| AT1G18740 | YES | | |
| AT1G19020 | YES | | |
| AT1G19025 | YES | | |
| AT1G19180 | YES | JAZ1 | jasmonate-zim-domain protein 1 |
| AT1G19190 | YES | | |
| AT1G19210 | YES | | |
| AT1G19770 | YES | ATPUP14 | purine permease 14 |
| AT1G20440 | YES | AtCOR47 | |
| AT1G20450 | YES | ERD1 | EARLY RESPONSIVE TO DEHYDRATION 1 |
| AT1G21850 | YES | sks8 | SKU5 similar 8 |
| AT1G22070 | YES | TGA3 | TGA1A-related gene 3 |
| AT1G22080 | YES | | |
| AT1G22190 | YES | RAP2.4 | related to AP2 4 |
| AT1G22200 | YES | | |
| AT1G22570 | YES | | |
| AT1G22830 | YES | | |
| AT1G22840 | YES | ATCYTC-A | CYTOCHROME C-A |
| AT1G23480 | YES | ATCSLA3 | cellulose synthase-like A3 |
| AT1G23710 | YES | | |
| AT1G25400 | YES | | |
| AT1G25550 | YES | | |
| AT1G25560 | YES | EDF1 | ETHYLENE RESPONSE DNA BINDING FACTOR 1 |
| AT1G27100 | YES | | |
| AT1G27720 | YES | TAF4 | TBP-associated factor 4 |
| AT1G27730 | YES | STZ | salt tolerance zinc finger |
| AT1G27760 | YES | ATSAT32 | SALT-TOLERANCE 32 |
| AT1G27770 | YES | ACA1 | autoinhibited Ca2+-ATPase 1 |
| AT1G28280 | YES | | |
| AT1G28480 | YES | GRX48 | |
| AT1G29395 | YES | COR413-TM1 | COLD REGULATED 314 THYLAKOID MEMBRANE 1 |
| AT1G29400 | YES | AML5 | MEI2-like protein 5 |
| AT1G29680 | YES | | |
| AT1G29690 | YES | CAD1 | constitutively activated cell death 1 |
| AT1G30135 | YES | JAZ8 | jasmonate-zim-domain protein 8 |
| AT1G30370 | YES | DLAH | DAD1-like acylhydrolase |
| AT1G30700 | YES | | |
| AT1G30740 | YES | | |
| AT1G31820 | YES | PUT1 | POLYAMINE UPTAKE TRANSPORTER 1 |
| AT1G32070 | YES | ATNSI | nuclear shuttle interacting |
| AT1G32640 | YES | ATMYC2 | |
| AT1G32920 | YES | | |
| AT1G32930 | YES | | |
| AT1G33590 | YES | | |
| AT1G35140 | YES | EXL1 | EXORDIUM like 1 |
| AT1G35910 | YES | TPPD | trehalose-6-phosphate phosphatase D |
| AT1G42560 | YES | ATMLO9 | ARABIDOPSIS THALIANA MILDEW RESISTANCE |
| | | | LOCUS O 9 |
| AT1G42990 | YES | ATBZIP6 | basic region/leucine zipper motif 6 |
| AT1G43160 | YES | RAP2.6 | related to AP2 6 |
| AT1G43900 | YES | | |
| AT1G43910 | YES | | |
| AT1G45145 | YES | ATH5 | THIOREDOXIN H-TYPE 5 |
| AT1G49520 | YES | | |
| AT1G50750 | YES | | |
| AT1G52890 | YES | ANAC19 | NAC domain containing protein 19 |
| AT1G53720 | YES | ATCYP59 | CYCLOPHILIN 59 |
| AT1G53830 | YES | ATPME2 | pectin methylesterase 2 |
| AT1G53840 | YES | ATPME1 | pectin methylesterase 1 |
| AT1G55450 | YES | | |
| AT1G56050 | YES | | |
| AT1G56060 | YES | | |
| AT1G56590 | YES | ZIP4 | ZIG SUPPRESSOR 4 |
| AT1G56660 | YES | | |
| AT1G56670 | YES | | |
| AT1G58210 | YES | EMB1674 | EMBRYO DEFECTIVE 1674 |
| AT1G58420 | YES | | |
| AT1G59590 | YES | ZCF37 | |
| AT1G59600 | YES | ZCW7 | |
| AT1G59870 | YES | ABCG36 | ATP-binding cassette G36 |
| AT1G60190 | YES | AtPUB19 | |
| AT1G61340 | YES | AtFBS1 | |
| AT1G61360 | YES | | |
| AT1G61820 | YES | BGLU46 | beta glucosidase 46 |
| AT1G61870 | YES | PPR336 | pentatricopeptide repeat 336 |
| AT1G61890 | YES | | |
| AT1G62300 | YES | ATWRKY6 | |
| AT1G62570 | YES | FMO GS-OX4 | flavin-monooxygenase glucosinolate S-oxygenase 4 |
| AT1G62790 | YES | | |
| AT1G64390 | YES | AtGH9C2 | glycosyl hydrolase 9C2 |
| AT1G64660 | YES | ATMGL | methionine gamma-lyase |
| AT1G64670 | YES | BDG1 | BODYGUARD1 |
| AT1G65510 | YES | | |
| AT1G65520 | YES | ATECI1 | ARABIDOPSIS THALIANA DELTA(3), DELTA(2)-ENOYL |
| | | | COA ISOMERASE 1 |
| AT1G66160 | YES | ATCMPG1 | |
| AT1G66170 | YES | MMD1 | MALE MEIOCYTE DEATH 1 |
| AT1G68440 | YES | | |
| AT1G68670 | YES | | |
| AT1G68760 | YES | ATNUDT1 | ARABIDOPSIS THALIANA NUDIX HYDROLASE |
| | | | HOMOLOG 1 |
| AT1G68765 | YES | IDA | INFLORESCENCE DEFICIENT IN ABSCISSION |
| AT1G68840 | YES | AtRAV2 | |
| AT1G69220 | YES | SIK1 | |
| AT1G69490 | YES | ANAC29 | Arabidopsis NAC domain containing protein 29 |
| AT1G69760 | YES | | |
| AT1G69880 | YES | ATH8 | thioredoxin H-type 8 |
| AT1G69890 | YES | | |
| AT1G69930 | YES | ATGSTU11 | glutathione S-transferase TAU 11 |
| AT1G70420 | YES | | |
| AT1G71530 | YES | | |
| AT1G71697 | YES | ATCK1 | choline kinase 1 |
| AT1G72520 | YES | ATLOX4 | Arabidopsis thaliana lipoxygenase 4 |
| AT1G73010 | YES | AtPPsPase1 | pyrophosphate-specific phosphatase1 |
| AT1G73080 | YES | ATPEPR1 | PEP1 RECEPTOR 1 |
| AT1G73500 | YES | ATMKK9 | |
| AT1G73510 | YES | | |
| AT1G73530 | YES | | |
| AT1G73540 | YES | atnudt21 | nudix hydrolase homolog 21 |
| AT1G74310 | YES | ATHSP11 | heat shock protein 11 |
| AT1G74450 | YES | | |
| AT1G74930 | YES | ORA47 | |
| AT1G76170 | YES | | |
| AT1G76180 | YES | ERD14 | EARLY RESPONSE TO DEHYDRATION 14 |
| AT1G76600 | YES | | |
| AT1G76640 | YES | | |
| AT1G76650 | YES | CML38 | calmodulin-like 38 |
| AT1G78080 | YES | RAP2.4 | related to AP2 4 |
| AT1G78290 | YES | SNRK2-8 | SNF1-RELATED PROTEIN KINASE 2-8 |
| AT1G78340 | YES | ATGSTU22 | glutathione S-transferase TAU 22 |
| AT1G79400 | YES | ATCHX2 | cation/H+ exchanger 2 |
| AT1G79990 | YES | | |
| AT1G80010 | YES | FRS8 | FAR1-related sequence 8 |
| AT1G80380 | YES | | |
| AT1G80820 | YES | ATCCR2 | |
| AT1G80840 | YES | ATWRKY4 | |
| AT1G80850 | YES | | |
| AT1G80930 | YES | | |
| AT2G01300 | YES | | |
| AT2G01670 | YES | atnudt17 | nudix hydrolase homolog 17 |
| AT2G03750 | YES | | |
| AT2G03760 | YES | AtSOT1 | |
| AT2G04040 | YES | ATDTX1 | |
| AT2G04050 | YES | | |
| AT2G04880 | YES | ATWRKY1 | |
| AT2G04890 | YES | SCL21 | SCARECROW-like 21 |
| AT2G05710 | YES | ACO3 | aconitase 3 |
| AT2G05720 | YES | | |
| AT2G05940 | YES | RIPK | RPM1-induced protein kinase |
| AT2G07050 | YES | CAS1 | cycloartenol synthase 1 |
| AT2G17080 | YES | | |
| AT2G17660 | YES | | |
| AT2G17670 | YES | | |
| AT2G17840 | YES | ERD7 | EARLY-RESPONSIVE TO DEHYDRATION 7 |
| AT2G18190 | YES | | |
| AT2G18210 | YES | | |
| AT2G18240 | YES | | |
| AT2G18690 | YES | | |
| AT2G20560 | YES | | |
| AT2G20570 | YES | ATGLK1 | ARABIDOPSIS GOLDEN2-LIKE 1 |
| AT2G22470 | YES | AGP2 | arabinogalactan protein 2 |
| AT2G22500 | YES | ATPUMP5 | PLANT UNCOUPLING MITOCHONDRIAL PROTEIN 5 |
| AT2G22760 | YES | | |
| AT2G22860 | YES | ATPSK2 | phytosulfokine 2 precursor |
| AT2G22870 | YES | EMB21 | embryo defective 21 |
| AT2G22880 | YES | | |
| AT2G23120 | YES | | |
| AT2G23170 | YES | GH3.3 | |
| AT2G23320 | YES | AtWRKY15 | |
| AT2G23810 | YES | TET8 | tetraspanin8 |
| AT2G24570 | YES | ATWRKY17 | |
| AT2G24850 | YES | TAT | TYROSINE AMINOTRANSFERASE |
| AT2G25460 | YES | | |
| AT2G25490 | YES | EBF1 | EIN3-binding F box protein 1 |
| AT2G25735 | YES | | |
| AT2G26530 | YES | AR781 | |
| AT2G26690 | YES | | |
| AT2G27080 | YES | | |
| AT2G27090 | YES | | |
| AT2G28400 | YES | | |
| AT2G29080 | YES | ftsh3 | FTSH protease 3 |
| AT2G29470 | YES | ATGSTU3 | glutathione S-transferase tau 3 |
| AT2G29480 | YES | ATGSTU2 | glutathione S-transferase tau 2 |
| AT2G29490 | YES | ATGSTU1 | glutathione S-transferase TAU 1 |
| AT2G30040 | YES | MAPKKK14 | mitogen-activated protein kinase kinase kinase 14 |
| AT2G30240 | YES | ATCHX13 | |
| AT2G30250 | YES | ATWRKY25 | |
| AT2G31690 | YES | | |
| AT2G32020 | YES | | |
| AT2G32120 | YES | HSP7T-2 | heat-shock protein 7T-2 |
| AT2G32150 | YES | | |
| AT2G32220 | YES | | |
| AT2G33710 | YES | | |
| AT2G34910 | YES | | |
| AT2G35410 | YES | | |
| AT2G35930 | YES | AtPUB23 | |
| AT2G35980 | YES | ATNHL1 | ARABIDOPSIS NDR1/HIN1-LIKE 1 |
| AT2G36220 | YES | | |
| AT2G36230 | YES | APG1 | ALBINO AND PALE GREEN 1 |
| AT2G36950 | YES | | |
| AT2G37430 | YES | ZAT11 | zinc finger of Arabidopsis thaliana 11 |
| AT2G37975 | YES | | |
| AT2G38240 | YES | | |
| AT2G38470 | YES | ATWRKY33 | WRKY DNA-BINDING PROTEIN 33 |
| AT2G38480 | YES | | |
| AT2G38830 | YES | | |
| AT2G39190 | YES | ATATH8 | |
| AT2G39200 | YES | ATMLO12 | MILDEW RESISTANCE LOCUS O 12 |
| AT2G39660 | YES | BIK1 | botrytis-induced kinase 1 |
| AT2G39670 | YES | | |
| AT2G39990 | YES | AteIF3f | Arabidopsis thaliana eukaryotic translation initiation factor 3 |
| | | | subunit F |
| AT2G40000 | YES | ATHSPRO2 | ARABIDOPSIS ORTHOLOG OF SUGAR BEET HS1 PRO-1 2 |
| AT2G40140 | YES | ATSZF2 | |
| AT2G41000 | YES | | |
| AT2G41010 | YES | ATCAMBP25 | calmodulin (CAM)-binding protein of 25 kDa |
| AT2G41100 | YES | ATCAL4 | ARABIDOPSIS THALIANA CALMODULIN LIKE 4 |
| AT2G41110 | YES | ATCAL5 | |
| AT2G41410 | YES | | |
| AT2G41430 | YES | CID1 | CTC-Interacting Domain 1 |
| AT2G41620 | YES | | |
| AT2G41630 | YES | TFIIB | transcription factor IIB |
| AT2G41640 | YES | | |
| AT2G41730 | YES | | |
| AT2G41740 | YES | ATVLN2 | |
| AT2G41790 | YES | | |
| AT2G41800 | YES | | |
| AT2G41890 | YES | | |
| AT2G43130 | YES | ARA-4 | |
| AT2G43290 | YES | MSS3 | multicopy suppressors of snf4 deficiency in yeast 3 |
| AT2G44790 | YES | UCC2 | uclacyanin 2 |
| AT2G44840 | YES | ATERF13 | ETHYLENE-RESPONSIVE ELEMENT BINDING FACTOR |
| | | | 13 |
| AT2G45400 | YES | BEN1 | |
| AT2G45810 | YES | | |
| AT2G45820 | YES | | |
| AT2G46140 | YES | | |
| AT2G46260 | YES | LRB1 | light-response BTB 1 |
| AT2G46390 | YES | SDH8 | succinate dehydrogenase 8 |
| AT2G46400 | YES | ATWRKY46 | WRKY DNA-BINDING PROTEIN 46 |
| AT2G46420 | YES | | |
| AT2G46830 | YES | AtCCA1 | |
| AT2G47000 | YES | ABCB4 | ATP-binding cassette B4 |
| AT2G47550 | YES | | |
| AT2G47950 | YES | | |
| AT3G01280 | YES | ATVDAC1 | ARABIDOPSIS THALIANA VOLTAGE DEPENDENT |
| | | | ANION CHANNEL 1 |
| AT3G01290 | YES | AtHIR2 | |
| AT3G01560 | YES | | |
| AT3G01830 | YES | | |
| AT3G01840 | YES | LYK2 | LysM-containing receptor-like kinase 2 |
| AT3G02040 | YES | AtGDPD1 | |
| AT3G02480 | YES | | |
| AT3G02840 | YES | | |
| AT3G02850 | YES | SKOR | STELAR K+ outward rectifier |
| AT3G02880 | YES | | |
| AT3G03810 | YES | EDA3 | embryo sac development arrest 3 |
| AT3G03890 | YES | | |
| AT3G04070 | YES | anac47 | NAC domain containing protein 47 |
| AT3G04120 | YES | GAPC | GLYCERALDEHYDE-3-PHOSPHATE DEHYDROGENASE |
| | | | C SUBUNIT |
| AT3G04130 | YES | | |
| AT3G04730 | YES | IAA16 | indoleacetic acid-induced protein 16 |
| AT3G05310 | YES | MIRO3 | MIRO-related GTP-ase 3 |
| AT3G06490 | YES | AtMYB18 | myb domain protein 18 |
| AT3G06500 | YES | A/N-InvC | alkaline/neutral invertase C |
| AT3G06510 | YES | ATSFR2 | SENSITIVE TO FREEZING 2 |
| AT3G08580 | YES | AAC1 | ADP/ATP carrier 1 |
| AT3G08590 | YES | iPGAM2 | 2,3-biphosphoglycerate-independent phosphoglycerate mutase 2 |
| AT3G08610 | YES | | |
| AT3G09440 | YES | | |
| AT3G09940 | YES | ATMDAR3 | ARABIDOPSIS THALIANA |
| | | | MONODEHYDROASCORBATE REDUCTASE 3 |
| AT3G10300 | YES | | |
| AT3G10920 | YES | ATMSD1 | ARABIDOPSIS MANGANESE SUPEROXIDE DISMUTASE 1 |
| AT3G10930 | YES | | |
| AT3G10985 | YES | ATWI-12 | ARABIDOPSIS THALIANA WOUND-INDUCED PROTEIN |
| | | | 12 |
| AT3G12120 | YES | FAD2 | fatty acid desaturase 2 |
| AT3G12320 | YES | | |
| AT3G13310 | YES | | |
| AT3G13320 | YES | atcax2 | |
| AT3G13790 | YES | ATBFRUCT1 | |
| AT3G13920 | YES | EIF4A1 | eukaryotic translation initiation factor 4A1 |
| AT3G14940 | YES | ATPPC3 | phosphoenolpyruvate carboxylase 3 |
| AT3G14990 | YES | AtDJ1A | DJ-1 homolog A |
| AT3G15210 | YES | ATERF-4 | ETHYLENE RESPONSIVE ELEMENT BINDING FACTOR 4 |
| AT3G15450 | YES | | |
| AT3G15460 | YES | | |
| AT3G15500 | YES | ANAC55 | NAC domain containing protein 55 |
| AT3G15620 | YES | UVR3 | UV REPAIR DEFECTIVE 3 |
| AT3G15630 | YES | | |
| AT3G16857 | YES | ARR1 | response regulator 1 |
| AT3G16860 | YES | COBL8 | COBRA-like protein 8 precursor |
| AT3G17390 | YES | MAT4 | METHIONINE ADENOSYLTRANSFERASE 4 |
| AT3G19020 | YES | | |
| AT3G19030 | YES | | |
| AT3G19240 | YES | | |
| AT3G19570 | YES | QWRF1 | QWRF domain containing 1 |
| AT3G19580 | YES | AZF2 | zinc-finger protein 2 |
| AT3G19930 | YES | ATSTP4 | SUGAR TRANSPORTER 4 |
| AT3G21070 | YES | ATNADK-1 | NAD KINASE 1 |
| AT3G21500 | YES | DXL1 | DXS-like 1 |
| AT3G22370 | YES | AOX1A | alternative oxidase 1A |
| AT3G22380 | YES | TIC | TIME FOR COFFEE |
| AT3G22900 | YES | NRPD7 | |
| AT3G22910 | YES | | |
| AT3G23170 | YES | | |
| AT3G23250 | YES | ATMYB15 | MYB DOMAIN PROTEIN 15 |
| AT3G23460 | YES | | |
| AT3G24050 | YES | GATA1 | GATA transcription factor 1 |
| AT3G24170 | YES | ATGR1 | glutathione-disulfide reductase |
| AT3G24550 | YES | ATPERK1 | proline-rich extensin-like receptor kinase 1 |
| AT3G24560 | YES | RSY3 | RASPBERRY 3 |
| AT3G25250 | YES | AGC2 | |
| AT3G25600 | YES | | |
| AT3G25610 | YES | | |
| AT3G25650 | YES | ASK15 | SKP1-like 15 |
| AT3G25655 | YES | IDL1 | inflorescence deficient in abscission (IDA)-like 1 |
| AT3G25780 | YES | AOC3 | allene oxide cyclase 3 |
| AT3G27510 | YES | | |
| AT3G28690 | YES | | |
| AT3G29010 | YES | | |
| AT3G29290 | YES | emb276 | embryo defective 276 |
| AT3G30775 | YES | AT-POX | |
| AT3G44260 | YES | AtCAF1a | CCR4-associated factor 1a |
| AT3G45730 | YES | | |
| AT3G45740 | YES | | |
| AT3G45970 | YES | ATEXLA1 | expansin-like A1 |
| AT3G45980 | YES | H2B | HISTONE H2B |
| AT3G46620 | YES | AtRDUF1 | Arabidopsis thaliana RING and Domain of Unknown Function |
| | | | 1117 1 |
| AT3G47340 | YES | ASN1 | glutamine-dependent asparagine synthase 1 |
| AT3G48520 | YES | CYP94B3 | cytochrome P45, family 94, subfamily B, polypeptide 3 |
| AT3G49000 | YES | | |
| AT3G49530 | YES | ANAC62 | NAC domain containing protein 62 |
| AT3G49780 | YES | ATPSK3 (FORMER SYMBOL) | |
| AT3G49790 | YES | | |
| AT3G50900 | YES | | |
| AT3G50910 | YES | | |
| AT3G50930 | YES | BCS1 | cytochrome BC1 synthesis |
| AT3G50960 | YES | PLP3a | phosducin-like protein 3 homolog |
| AT3G50970 | YES | LTI3 | LOW TEMPERATURE-INDUCED 3 |
| AT3G50980 | YES | XERO1 | dehydrin xero 1 |
| AT3G51920 | YES | ATCML9 | |
| AT3G52450 | YES | AtPUB22 | |
| AT3G52700 | YES | | |
| AT3G52710 | YES | | |
| AT3G52800 | YES | | |
| AT3G52810 | YES | ATPAP21 | PURPLE ACID PHOSPHATASE 21 |
| AT3G52930 | YES | AtFBA8 | |
| AT3G53480 | YES | ABCG37 | ATP-binding cassette G37 |
| AT3G53510 | YES | ABCG2 | ATP-binding cassette G2 |
| AT3G53600 | YES | | |
| AT3G53610 | YES | ATRAB8 | RAB GTPase homolog 8 |
| AT3G53760 | YES | ATGCP4 | |
| AT3G54150 | YES | | |
| AT3G55440 | YES | ATCTIMC | CYTOSOLIC TRIOSE PHOSPHATE ISOMERASE |
| AT3G55620 | YES | eIF6A | eukaryotic initiation facor 6A |
| AT3G55630 | YES | ATDFD | DHFS-FPGS homolog D |
| AT3G55640 | YES | | |
| AT3G55970 | YES | ATJRG21 | |
| AT3G55980 | YES | ATSZF1 | |
| AT3G56800 | YES | ACAM-3 | CALMODULIN 3 |
| AT3G56880 | YES | | |
| AT3G57450 | YES | | |
| AT3G57460 | YES | | |
| AT3G59350 | YES | | |
| AT3G59360 | YES | ATUTR6 | UDP-GALACTOSE TRANSPORTER 6 |
| AT3G60130 | YES | BGLU16 | beta glucosidase 16 |
| AT3G60140 | YES | BGLU3 | BETA GLUCOSIDASE 3 |
| AT3G61190 | YES | BAP1 | BON association protein 1 |
| AT3G61640 | YES | AGP2 | arabinogalactan protein 2 |
| AT3G61890 | YES | ATHB-12 | homeobox 12 |
| AT3G62260 | YES | | |
| AT3G62410 | YES | CP12 | CP12 DOMAIN-CONTAINING PROTEIN 1 |
| AT3G63380 | YES | | |
| AT4G00170 | YES | | |
| AT4G00690 | YES | ULP1B | UB-like protease 1B |
| AT4G01370 | YES | ATMPK4 | MAP kinase 4 |
| AT4G02380 | YES | AtLEA5 | Arabidopsis thaliana late embryogenensis abundant like 5 |
| AT4G02880 | YES | | |
| AT4G04500 | YES | CRK37 | cysteine-rich RLK (RECEPTOR-like protein kinase) 37 |
| AT4G05050 | YES | UBQ11 | ubiquitin 11 |
| AT4G05100 | YES | AtMYB74 | myb domain protein 74 |
| AT4G05320 | YES | UBI1 | ubiquitin 1 |
| AT4G08850 | YES | | |
| AT4G08950 | YES | EXO | EXORDIUM |
| AT4G09630 | YES | | |
| AT4G11280 | YES | ACS6 | 1-aminocyclopropane-1-carboxylic acid (acc) synthase 6 |
| AT4G11350 | YES | | |
| AT4G11360 | YES | RHA1B | RING-H2 finger A1B |
| AT4G11560 | YES | | |
| AT4G11570 | YES | | |
| AT4G11670 | YES | | |
| AT4G12720 | YES | AtNUDT7 | Arabidopsis thaliana Nudix hydrolase homolog 7 |
| AT4G12730 | YES | FLA2 | FASCICLIN-like arabinogalactan 2 |
| AT4G13390 | YES | EXT12 | extensin 12 |
| AT4G15610 | YES | | |
| AT4G16670 | YES | | |
| AT4G16680 | YES | | |
| AT4G16820 | YES | PLA-I{beta]2 | phospholipase A I beta 2 |
| AT4G16830 | YES | | |
| AT4G17490 | YES | ATERF6 | ethylene responsive element binding factor 6 |
| AT4G17500 | YES | ATERF-1 | ethylene responsive element binding factor 1 |
| AT4G17520 | YES | | |
| AT4G17615 | YES | ATCBL1 | ARABIDOPSIS THALIANA CALCINEURIN B-LIKE |
| | | | PROTEIN |
| AT4G18170 | YES | ATWRKY28 | |
| AT4G18880 | YES | AT-HSFA4A | ARABIDOPSIS THALIANA HEAT SHOCK |
| | | | TRANSCRIPTION FACTOR A4A |
| AT4G19200 | YES | | |
| AT4G19210 | YES | ABCE2 | ATP-binding cassette E2 |
| AT4G20000 | YES | | |
| AT4G20830 | YES | | |
| AT4G20840 | YES | | |
| AT4G20860 | YES | | |
| AT4G20870 | YES | ATFAH2 | ARABIDOPSIS FATTY ACID HYDROXYLASE 2 |
| AT4G21120 | YES | AAT1 | amino acid transporter 1 |
| AT4G21490 | YES | NDB3 | NAD(P)H dehydrogenase B3 |
| AT4G21820 | YES | | |
| AT4G21850 | YES | ATMSRB9 | methionine sulfoxide reductase B9 |
| AT4G22720 | YES | | |
| AT4G23190 | YES | AT-RLK3 | RECEPTOR LIKE PROTEIN KINASE 3 |
| AT4G24390 | YES | AFB4 | auxin signaling F-box 4 |
| AT4G24570 | YES | DIC2 | dicarboxylate carrier 2 |
| AT4G24580 | YES | REN1 | ROP1 ENHANCER 1 |
| AT4G25570 | YES | ACYB-2 | |
| AT4G25580 | YES | | |
| AT4G25810 | YES | XTH23 | xyloglucan endotransglucosylase/hydrolase 23 |
| AT4G25820 | YES | ATXTH14 | |
| AT4G26040 | YES | | |
| AT4G26180 | YES | | |
| AT4G27270 | YES | | |
| AT4G27280 | YES | | |
| AT4G27580 | YES | | |
| AT4G27652 | YES | | |
| AT4G27654 | YES | | |
| AT4G27657 | YES | | |
| AT4G28460 | YES | | |
| AT4G29780 | YES | | |
| AT4G29790 | YES | | |
| AT4G30210 | YES | AR2 | |
| AT4G30280 | YES | ATXTH18 | XYLOGLUCAN |
| | | | ENDOTRANSGLUCOSYLASE/HYDROLASE 18 |
| AT4G30290 | YES | ATXTH19 | XYLOGLUCAN |
| | | | ENDOTRANSGLUCOSYLASE/HYDROLASE 19 |
| AT4G30430 | YES | TET9 | tetraspanin9 |
| AT4G30440 | YES | GAE1 | UDP-D-glucuronate 4-epimerase 1 |
| AT4G30530 | YES | GGP1 | gamma-glutamyl peptidase 1 |
| AT4G30600 | YES | | |
| AT4G31550 | YES | ATWRKY11 | |
| AT4G31800 | YES | ATWRKY18 | ARABIDOPSIS THALIANA WRKY DNA-BINDING |
| | | | PROTEIN 18 |
| AT4G31805 | YES | | |
| AT4G32020 | YES | | |
| AT4G32920 | YES | | |
| AT4G33666 | YES | | |
| AT4G33670 | YES | | |
| AT4G33780 | YES | | |
| AT4G33920 | YES | | |
| AT4G33925 | YES | SSN2 | suppressor of sni1 2 |
| AT4G33950 | YES | ATOST1 | OPEN STOMATA 1 |
| AT4G34150 | YES | | |
| AT4G34160 | YES | CYCD3 | |
| AT4G34410 | YES | RRTF1 | redox responsive transcription factor 1 |
| AT4G35580 | YES | CBNAC | calmodulin-binding NAC protein |
| AT4G36010 | YES | | |
| AT4G36040 | YES | J11 | DnaJ11 |
| AT4G36500 | YES | | |
| AT4G36640 | YES | | |
| AT4G37010 | YES | CEN2 | centrin 2 |
| AT4G37260 | YES | ATMYB73 | |
| AT4G37270 | YES | ATHMA1 | ARABIDOPSIS THALIANA HEAVY METAL ATPASE 1 |
| AT4G37370 | YES | CYP81D8 | cytochrome P45, family 81, subfamily D, polypeptide 8 |
| AT4G37590 | YES | MEL1 | MAB4/ENP/NPY1-LIKE 1 |
| AT4G37610 | YES | BT5 | BTB and TAZ domain protein 5 |
| AT4G37770 | YES | ACS8 | 1-amino-cyclopropane-1-carboxylate synthase 8 |
| AT4G37900 | YES | | |
| AT4G37910 | YES | mtHsc7-1 | mitochondrial heat shock protein 7-1 |
| AT4G38420 | YES | sks9 | SKU5 similar 9 |
| AT4G39080 | YES | VHA-A3 | vacuolar proton ATPase A3 |
| AT4G39090 | YES | RD19 | RESPONSIVE TO DEHYDRATION 19 |
| AT4G39260 | YES | ATGRP8 | GLYCINE-RICH PROTEIN 8 |
| AT4G39640 | YES | GGT1 | gamma-glutamyl transpeptidase 1 |
| AT4G40030 | YES | | |
| AT4G40040 | YES | | |
| AT5G01380 | YES | | |
| AT5G01500 | YES | TAAC | thylakoid ATP/ADP carrier |
| AT5G01510 | YES | RUS5 | ROOT UV-B SENSITIVE 5 |
| AT5G01540 | YES | LecRK-VI.2 | L-type lectin receptor kinase-VI.2 |
| AT5G01600 | YES | ATFER1 | ARABIDOPSIS THALIANA FERRETIN 1 |
| AT5G01750 | YES | | |
| AT5G01820 | YES | ATCIPK14 | |
| AT5G01950 | YES | | |
| AT5G01960 | YES | | |
| AT5G02020 | YES | SIS | Salt Induced Serine rich |
| AT5G02230 | YES | | |
| AT5G02240 | YES | | |
| AT5G02810 | YES | APRR7 | |
| AT5G02820 | YES | BIN5 | BRASSINOSTEROID INSENSITIVE 5 |
| AT5G03210 | YES | AtDIP2 | |
| AT5G03380 | YES | | |
| AT5G03610 | YES | | |
| AT5G04330 | YES | CYP84A4 | CYTOCHROME P45 84A4 |
| AT5G04750 | YES | | |
| AT5G05410 | YES | DREB2 | DEHYDRATION-RESPONSIVE ELEMENT BINDING |
| | | | PROTEIN 2 |
| AT5G05420 | YES | | |
| AT5G05600 | YES | | |
| AT5G05790 | YES | | |
| AT5G06290 | YES | 2-Cys Prx B | 2-cysteine peroxiredoxin B |
| AT5G06300 | YES | LOG7 | LONELY GUY 7 |
| AT5G06320 | YES | NHL3 | NDR1/HIN1-like 3 |
| AT5G07440 | YES | GDH2 | glutamate dehydrogenase 2 |
| AT5G07450 | YES | CYCP4; 3 | cyclin p4; 3 |
| AT5G07730 | YES | | |
| AT5G07740 | YES | | |
| AT5G08230 | YES | | |
| AT5G08240 | YES | | |
| AT5G09990 | YES | PROPEP5 | elicitor peptide 5 precursor |
| AT5G10180 | YES | AST68 | ARABIDOPSIS SULFATE TRANSPORTER 68 |
| AT5G10630 | YES | | |
| AT5G10690 | YES | | |
| AT5G10695 | YES | | |
| AT5G10700 | YES | | |
| AT5G10710 | YES | | |
| AT5G11090 | YES | | |
| AT5G11650 | YES | | |
| AT5G11670 | YES | ATNADP-ME2 | Arabidopsis thaliana NADP-malic enzyme 2 |
| AT5G11740 | YES | AGP15 | arabinogalactan protein 15 |
| AT5G12340 | YES | | |
| AT5G13200 | YES | | |
| AT5G13220 | YES | JAS1 | JASMONATE-ASSOCIATED 1 |
| AT5G13470 | YES | | |
| AT5G14730 | YES | | |
| AT5G14740 | YES | BETA CA2 | BETA CARBONIC ANHYDRASE 2 |
| AT5G15090 | YES | ATVDAC3 | ARABIDOPSIS THALIANA VOLTAGE DEPENDENT |
| | | | ANION CHANNEL 3 |
| AT5G15130 | YES | ATWRKY72 | ARABIDOPSIS THALIANA WRKY DNA-BINDING |
| | | | PROTEIN 72 |
| AT5G15980 | YES | | |
| AT5G17330 | YES | GAD | glutamate decarboxylase |
| AT5G17350 | YES | | |
| AT5G17360 | YES | | |
| AT5G17460 | YES | | |
| AT5G17650 | YES | | |
| AT5G18270 | YES | ANAC87 | Arabidopsis NAC domain containing protein 87 |
| AT5G18310 | YES | | |
| AT5G18475 | YES | | |
| AT5G19110 | YES | | |
| AT5G19240 | YES | | |
| AT5G20150 | YES | ATSPX1 | ARABIDOPSIS THALIANA SPX DOMAIN GENE 1 |
| AT5G20230 | YES | ATBCB | blue-copper-binding protein |
| AT5G20240 | YES | PI | PISTILLATA |
| AT5G24590 | YES | ANAC91 | Arabidopsis NAC domain containing protein 91 |
| AT5G24650 | YES | | |
| AT5G24800 | YES | ATBZIP9 | ARABIDOPSIS THALIANA BASIC LEUCINE ZIPPER 9 |
| AT5G24930 | YES | ATCOL4 | |
| AT5G25280 | YES | | |
| AT5G25930 | YES | | |
| AT5G26030 | YES | ATFC-I | |
| AT5G26340 | YES | ATSTP13 | SUGAR TRANSPORT PROTEIN 13 |
| AT5G26360 | YES | | |
| AT5G26760 | YES | | |
| AT5G27420 | YES | ATL31 | Arabidopsis toxicos en levadura 31 |
| AT5G35735 | YES | | |
| AT5G36260 | YES | | |
| AT5G37500 | YES | GORK | gated outwardly-rectifying K+ channel |
| AT5G37770 | YES | CML24 | CALMODULIN-LIKE 24 |
| AT5G39580 | YES | | |
| AT5G39670 | YES | | |
| AT5G39680 | YES | EMB2744 | EMBRYO DEFECTIVE 2744 |
| AT5G40690 | YES | | |
| AT5G40780 | YES | LHT1 | lysine histidine transporter 1 |
| AT5G41080 | YES | AtGDPD2 | |
| AT5G41810 | YES | | |
| AT5G42050 | YES | | |
| AT5G42370 | YES | | |
| AT5G42380 | YES | CML37 | calmodulin like 37 |
| AT5G42830 | YES | | |
| AT5G43440 | YES | | |
| AT5G43450 | YES | | |
| AT5G43580 | YES | UPI | UNUSUAL SERINE PROTEASE INHIBITOR |
| AT5G44320 | YES | | |
| AT5G44330 | YES | | |
| AT5G45110 | YES | ATNPR3 | |
| AT5G45140 | YES | NRPC2 | nuclear RNA polymerase C2 |
| AT5G45350 | YES | | |
| AT5G45630 | YES | | |
| AT5G46780 | YES | | |
| AT5G47200 | YES | ATRAB1A | RAB GTPase homolog 1A |
| AT5G47210 | YES | | |
| AT5G47220 | YES | ATERF-2 | ETHYLENE RESPONSE FACTOR-2 |
| AT5G47230 | YES | ATERF-5 | ETHYLENE RESPONSIVE ELEMENT BINDING FACTOR-5 |
| AT5G47910 | YES | ATRBOHD | |
| AT5G47960 | YES | ATRABA4C | RAB GTPase homolog A4C |
| AT5G47970 | YES | | |
| AT5G49030 | YES | OVA2 | ovule abortion 2 |
| AT5G49220 | YES | | |
| AT5G49480 | YES | ATCP1 | Ca2+-binding protein 1 |
| AT5G49520 | YES | ATWRKY48 | ARABIDOPSIS THALIANA WRKY DNA-BINDING |
| | | | PROTEIN 48 |
| AT5G50900 | YES | | |
| AT5G52050 | YES | | |
| AT5G52400 | YES | CYP715A1 | cytochrome P45, family 715, subfamily A, polypeptide 1 |
| AT5G52410 | YES | | |
| AT5G52750 | YES | | |
| AT5G53110 | YES | | |
| AT5G54490 | YES | PBP1 | pinoid-binding protein 1 |
| AT5G55140 | YES | | |
| AT5G55780 | YES | | |
| AT5G56340 | YES | ATCRT1 | |
| AT5G56980 | YES | | |
| AT5G57190 | YES | PSD2 | phosphatidylserine decarboxylase 2 |
| AT5G57500 | YES | | |
| AT5G57510 | YES | | |
| AT5G57550 | YES | XTH25 | xyloglucan endotransglucosylase/hydrolase 25 |
| AT5G57560 | YES | TCH4 | Touch 4 |
| AT5G57720 | YES | | |
| AT5G58060 | YES | ATGP1 | |
| AT5G58070 | YES | ATTIL | TEMPERATURE-INDUCED LIPOCALIN |
| AT5G59450 | YES | | |
| AT5G59490 | YES | | |
| AT5G59820 | YES | AtZAT12 | |
| AT5G59830 | YES | | |
| AT5G61520 | YES | | |
| AT5G61890 | YES | | |
| AT5G61910 | YES | | |
| AT5G62520 | YES | SRO5 | similar to RCD one 5 |
| AT5G62530 | YES | ALDH12A1 | aldehyde dehydrogenase 12A1 |
| AT5G63130 | YES | | |
| AT5G63780 | YES | SHA1 | shoot apical meristem arrest 1 |
| AT5G63790 | YES | ANAC12 | NAC domain containing protein 12 |
| AT5G64120 | YES | | |
| AT5G64240 | YES | AtMC3 | metacaspase 3 |
| AT5G64310 | YES | AGP1 | arabinogalactan protein 1 |
| AT5G64650 | YES | | |
| AT5G64660 | YES | ATCMPG2 | |
| AT5G64905 | YES | PROPEP3 | elicitor peptide 3 precursor |
| AT5G65205 | YES | | |
| AT5G65300 | YES | | |
| AT5G65660 | YES | | |
| AT5G66055 | YES | AKRP | ankyrin repeat protein |
| AT5G66060 | YES | | |
| AT5G66460 | YES | AtMAN7 | |
| AT5G67080 | YES | MAPKKK19 | mitogen-activated protein kinase kinase kinase 19 |
| AT5G67300 | YES | ATMYB44 | ARABIDOPSIS THALIANA MYB DOMAIN PROTEIN 44 |
| AT5G67310 | YES | CYP81G1 | cytochrome P45, family 81, subfamily G, polypeptide 1 |
| AT5G67420 | YES | ASL39 | ASYMMETRIC LEAVES2-LIKE 39 |
| AT5G67560 | YES | ARLA1D | ADP-ribosylation factor-like A1D |
| AT1G01471 | NO | | |
| AT1G02520 | NO | ABCB11 | ATP-binding cassette B11 |
| AT1G02530 | NO | ABCB12 | ATP-binding cassette B12 |
| AT1G02590 | NO | | |
| AT1G02600 | NO | | |
| AT1G02920 | NO | ATGST11 | ARABIDOPSIS GLUTATHIONE S-TRANSFERASE 11 |
| AT1G02930 | NO | ATGST1 | ARABIDOPSIS GLUTATHIONE S-TRANSFERASE 1 |
| AT1G03220 | NO | | |
| AT1G05320 | NO | | |
| AT1G05675 | NO | | |
| AT1G07135 | NO | | |
| AT1G08950 | NO | | |
| AT1G09690 | NO | | |
| AT1G09932 | NO | | |
| AT1G10155 | NO | ATPP2-A1 | phloem protein 2-A1 |
| AT1G11550 | NO | | |
| AT1G13350 | NO | | |
| AT1G13360 | NO | | |
| AT1G14549 | NO | | |
| AT1G14870 | NO | AtPCR2 | |
| AT1G15015 | NO | | |
| AT1G15030 | NO | | |
| AT1G15045 | NO | | |
| AT1G15090 | NO | | |
| AT1G16635 | NO | | |
| AT1G17147 | NO | | |
| AT1G18200 | NO | AtRABA6b | RAB GTPase homolog A6B |
| AT1G18300 | NO | atnudt4 | nudix hydrolase homolog 4 |
| AT1G18745 | NO | | |
| AT1G21395 | NO | | |
| AT1G24160 | NO | | |
| AT1G27695 | NO | | |
| AT1G29640 | NO | | |
| AT1G30720 | NO | | |
| AT1G30730 | NO | | |
| AT1G32928 | NO | | |
| AT1G42980 | NO | | |
| AT1G49610 | NO | | |
| AT1G53625 | NO | | |
| AT1G55340 | NO | | |
| AT1G56240 | NO | AtPP2-B13 | phloem protein 2-B13 |
| AT1G56242 | NO | | |
| AT1G57690 | NO | | |
| AT1G57980 | NO | | |
| AT1G57990 | NO | ATPUP18 | purine permease 18 |
| AT1G61880 | NO | | |
| AT1G62870 | NO | | |
| AT1G68770 | NO | | |
| AT1G68845 | NO | | |
| AT1G69130 | NO | | |
| AT1G69290 | NO | | |
| AT1G69300 | NO | | |
| AT1G70390 | NO | | |
| AT1G70780 | NO | | |
| AT1G70782 | NO | CPuORF28 | conserved peptide upstream open reading frame 28 |
| AT1G71520 | NO | | |
| AT1G71528 | NO | | |
| AT1G74929 | NO | | |
| AT1G76680 | NO | ATOPR1 | ARABIDOPSIS 12-OXOPHYTODIENOATE REDUCTASE 1 |
| AT1G76690 | NO | ATOPR2 | ARABIDOPSIS 12-OXOPHYTODIENOATE REDUCTASE 2 |
| AT1G78830 | NO | | |
| AT1G78850 | NO | | |
| AT1G79240 | NO | | |
| AT1G79980 | NO | | |
| AT2G07772 | NO | | |
| AT2G17190 | NO | | |
| AT2G17830 | NO | | |
| AT2G18193 | NO | | |
| AT2G19260 | NO | | |
| AT2G20562 | NO | | |
| AT2G23118 | NO | | |
| AT2G23321 | NO | | |
| AT2G25130 | NO | | |
| AT2G30020 | NO | | |
| AT2G31030 | NO | ORP1B | OSBP(oxysterol binding protein)-related protein 1B |
| AT2G31345 | NO | | |
| AT2G32190 | NO | | |
| AT2G32210 | NO | | |
| AT2G36770 | NO | | |
| AT2G36800 | NO | DOGT1 | don-glucosyltransferase 1 |
| AT2G38230 | NO | ATPDX1.1 | ARABIDOPSIS THALIANA PYRIDOXINE BIOSYNTHESIS |
| | | | 1.1 |
| AT2G38823 | NO | | |
| AT2G41415 | NO | | |
| AT2G41440 | NO | | |
| AT2G43120 | NO | | |
| AT2G45390 | NO | | |
| AT2G45950 | NO | ASK2 | SKP1-like 2 |
| AT2G46995 | NO | | |
| AT3G02030 | NO | | |
| AT3G02468 | NO | CPuORF9 | conserved peptide upstream open reading frame 9 |
| AT3G02470 | NO | SAMDC | S-adenosylmethionine decarboxylase |
| AT3G10815 | NO | | |
| AT3G10986 | NO | | |
| AT3G11950 | NO | | |
| AT3G13080 | NO | ABCC3 | ATP-binding cassette C3 |
| AT3G13300 | NO | VCS | VARICOSE |
| AT3G13432 | NO | | |
| AT3G13600 | NO | | |
| AT3G14362 | NO | DVL19 | DEVIL 19 |
| AT3G18950 | NO | | |
| AT3G18952 | NO | | |
| AT3G23470 | NO | | |
| AT3G25597 | NO | | |
| AT3G29000 | NO | | |
| AT3G30770 | NO | | |
| AT3G46080 | NO | | |
| AT3G46090 | NO | ZAT7 | |
| AT3G47790 | NO | ABCA8 | ATP-binding cassette A8 |
| AT3G48515 | NO | | |
| AT3G49570 | NO | LSU3 | RESPONSE TO LOW SULFUR 3 |
| AT3G49796 | NO | | |
| AT3G56790 | NO | | |
| AT3G62420 | NO | ATBZIP53 | basic region/leucine zipper motif 53 |
| AT3G62422 | NO | CPuORF3 | conserved peptide upstream open reading frame 3 |
| AT4G01360 | NO | BPS3 | BYPASS 3 |
| AT4G03635 | NO | | |
| AT4G05048 | NO | U49.1 | |
| AT4G08555 | NO | | |
| AT4G09040 | NO | | |
| AT4G12731 | NO | | |
| AT4G12735 | NO | | |
| AT4G13395 | NO | DVL1 | DEVIL 1 |
| AT4G15760 | NO | MO1 | monooxygenase 1 |
| AT4G17616 | NO | | |
| AT4G20920 | NO | | |
| AT4G21830 | NO | ATMSRB7 | methionine sulfoxide reductase B7 |
| AT4G21910 | NO | | |
| AT4G21920 | NO | | |
| AT4G22590 | NO | TPPG | trehalose-6-phosphate phosphatase G |
| AT4G22592 | NO | CPuORF27 | conserved peptide upstream open reading frame 27 |
| AT4G22710 | NO | CYP76A2 | cytochrome P45, family 76, subfamily A, polypeptide 2 |
| AT4G23550 | NO | ATWRKY29 | |
| AT4G23560 | NO | AtGH9B15 | glycosyl hydrolase 9B15 |
| AT4G24565 | NO | | |
| AT4G27585 | NO | | |
| AT4G28470 | NO | ATRPN1B | |
| AT4G32480 | NO | | |
| AT4G34131 | NO | UGT73B3 | UDP-glucosyl transferase 73B3 |
| AT4G34412 | NO | | |
| AT4G36648 | NO | | |
| AT4G37390 | NO | AUR3 | AUXIN UPREGULATED 3 |
| AT4G37608 | NO | | |
| AT5G01542 | NO | | |
| AT5G01595 | NO | | |
| AT5G02815 | NO | | |
| AT5G03204 | NO | | |
| AT5G06310 | NO | AtPOT1b | protection of telomeres 1b |
| AT5G06990 | NO | | |
| AT5G08770 | NO | | |
| AT5G08780 | NO | | |
| AT5G08790 | NO | anac81 | Arabidopsis NAC domain containing protein 81 |
| AT5G13210 | NO | | |
| AT5G15960 | NO | KIN1 | |
| AT5G15970 | NO | AtCor6.6 | |
| AT5G18480 | NO | PGSIP6 | plant glycogenin-like starch initiation protein 6 |
| AT5G19230 | NO | | |
| AT5G20010 | NO | ATRAN1 | ARABIDOPSIS THALIANA RAS-RELATED NUCLEAR |
| | | | PROTEIN |
| AT5G20225 | NO | | |
| AT5G21930 | NO | ATHMA8 | ARABIDOPSIS HEAVY METAL ATPASE 8 |
| AT5G21940 | NO | | |
| AT5G24630 | NO | BIN4 | brassinosteroid-insensitive4 |
| AT5G24640 | NO | | |
| AT5G36920 | NO | | |
| AT5G39581 | NO | | |
| AT5G40700 | NO | | |
| AT5G40880 | NO | | |
| AT5G42053 | NO | | |
| AT5G43570 | NO | | |
| AT5G43620 | NO | | |
| AT5G43650 | NO | BHLH92 | |
| AT5G47229 | NO | | |
| AT5G51730 | NO | | |
| AT5G53300 | NO | UBC1 | ubiquitin-conjugating enzyme 1 |
| AT5G53588 | NO | CPuORF5 | conserved peptide upstream open reading frame 5 |
| AT5G53590 | NO | | |
| AT5G53592 | NO | | |
| AT5G54100 | NO | | |
| AT5G55870 | NO | | |
| AT5G56975 | NO | | |
| AT5G57010 | NO | | |
| AT5G57015 | NO | ckl12 | casein kinase I-like 12 |
| AT5G61900 | NO | BON | |
| AT5G64320 | NO | | |
| AT5G64401 | NO | | |
| AT5G65207 | NO | | |
| AT5G65687 | NO | | |
| AT5G65690 | NO | PCK2 | phosphoenolpyruvate carboxykinase 2 |

Integration of TF-Regulation and TF-Binding Data Identifies Three Modes-of-Action for bZIP1 and its Primary Targets: Poised, Stable, and Transient.

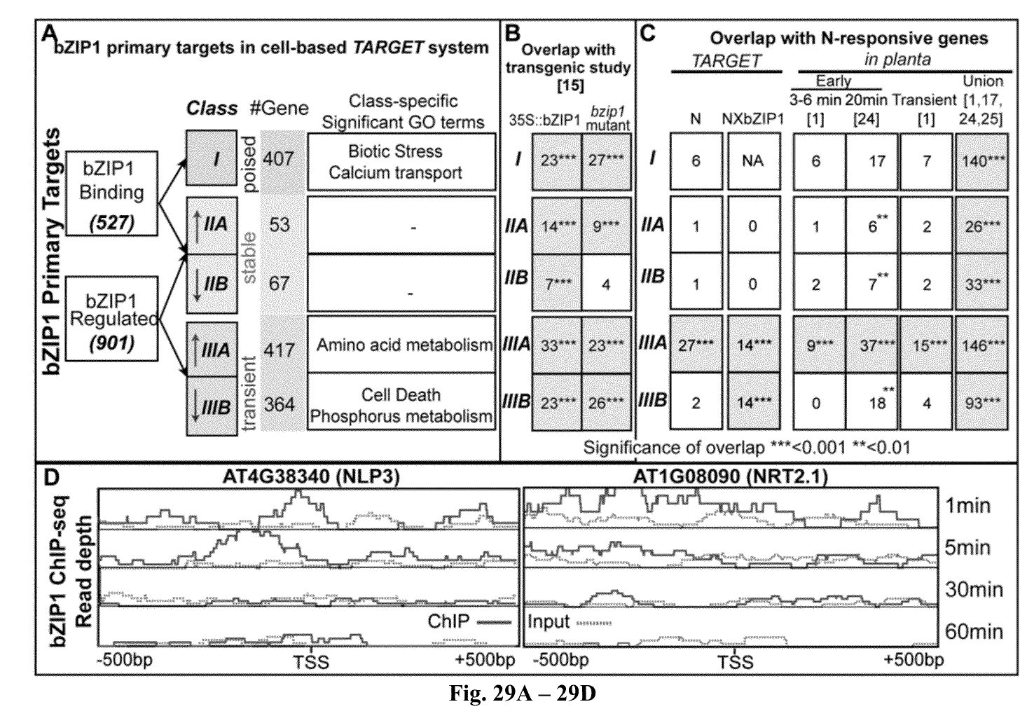

To understand the underlying mechanisms by which bZIP1 propagates N-signals through a GRN, primary targets identified either by TF-induced gene regulation or TF-binding were integrated. To enable a direct comparison of transcriptome and TF-binding data, of the 850 genes bound to bZIP1, 187 genes not represented on the ATH1 microarray were omitted. 136 genes that did not pass the stringent filters for effects of protoplasting, DEX, or CHX treatment were also omitted. This resulted in a filtered total of 527 bZIP1 bound genes (FIG. 29A). The resulting list of 1,308 high-confidence primary targets of bZIP1 identified either by TF-mediated gene regulation (901 genes) or TF-binding (527 genes) were integrated and analyzed for biological relevance to the N-signal (FIG. 29). The intersection of the TF-regulation and TF-binding data identified three classes of primary targets, representing distinct modes-of-action for bZIP1 in N-signal propagation (FIG. 29A; Table 19). Class I targets (407 genes) were deemed “Poised”, as they are bound to bZIP1 but show no significant TF-induced gene regulation. Class II targets (120 genes), are deemed “Stable”, as they are both bound and regulated by bZIP1. Unexpectedly, Class III targets (781 genes)—the largest class of bZIP1 primary target genes—were deemed “Transient” as they are regulated by bZIP1 perturbation, but not detectably bound to it. We note that these are not indirect TF targets, as ChIP-seq is able to detect direct or indirect binding by bZIP1, i.e., as part of a protein complex. They also cannot be dismissed as secondary targets of bZIP1, as they are regulated in response to DEX-induced bZIP1 perturbation performed in the presence of CHX, which blocks the regulation of secondary targets.

TABLE 19
Classes of bZIP1 primary targets: Class I, Poised; Class II Stable (IIA induced;
IIB repressed); and Class III transient (IIIA induced, IIIB repressed) listed as 5 subclasses.
Gene annotations are from TAIR10.
Class I. BN: Bind but no regulation

| | | |
|---|---|---|
| At1g14560 | Mitochondrial substrate carrier family protein | Class I |
| At2g23120 | Late embryogenesis abundant protein, group 6 | Class I |
| At5g57720 | AP2/B3-like transcriptional factor family protein | Class I |
| At5g02820 | BIN5, RHL2, Spo1 1/DNA topoisomerase VI, subunit A protein | Class I |
| At4g09630 | Protein of unknown function (DUF616) | Class I |
| At3g52700 | unknown protein; Has 6 Blast hits to 6 proteins in 2 species: Archae - 0; Bacteria - 0; Metazoa - | Class I |
| | 0; Fungi - 0; Plants - 6; Viruses - 0; Other Eukaryotes - 0 (source: NCBI BLink). | |
| At1g16640 | AP2/B3-like transcriptional factor family protein | Class I |
| At3g10920 | ATMSD1, MEE33, MSD1, manganese superoxide dismutase 1 | Class I |
| At1g61820 | BGLU46, beta glucosidase 46 | Class I |
| At4g39080 | VHA-A3, vacuolar proton ATPase A3 | Class I |
| At1g53720 | ATCYP59, CYP59, cyclophilin 59 | Class I |
| At3g29290 | emb2076, Pentatricopeptide repeat (PPR) superfamily protein | Class I |
| At1g64390 | AtGH9C2, GH9C2, glycosyl hydrolase 9C2 | Class I |
| At5g01500 | TAAC, thylakoid ATP/ADP carrier | Class I |
| At3g45980 | H2B, HTB9, Histone superfamily protein | Class I |
| At1g32920 | unknown protein; FUNCTIONS IN: molecular_function unknown; INVOLVED IN: response | Class I |
| | to wounding; LOCATED IN: endomembrane system; EXPRESSED IN: 23 plant structures; | |
| | EXPRESSED DURING: 13 growth stages; BEST Arabidopsis thaliana protein match is: | |
| | unknown protein (TAIR: AT1G32928.1); Has 42 Blast hits to 42 proteins in 8 species: Archae - | |
| | 0; Bacteria - 0; Metazoa - 0; Fungi - 0; Plants - 42; Viruses - 0; Other Eukaryotes - 0 (source: | |
| | NCBI BLink). | |
| At4g23190 | AT-RLK3, CRK11, cysteine-rich RLK (RECEPTOR-like protein kinase) 11 | Class I |
| At2g36230 | APG10, HISN3, Aldolase-type TIM barrel family protein | Class I |
| At2g26690 | Major facilitator superfamily protein | Class I |
| At1g73080 | ATPEPR1, PEPR1, PEP1 receptor 1 | Class I |
| At4g35580 | NTL9, NAC transcription factor-like 9 | Class I |
| At4g33950 | ATOST1, OST1, P44, SNRK2-6, SNRK2.6, SRK2E, Protein kinase superfamily protein | Class I |
| At5g67560 | ARLA1D, ATARLA1D, ADP-ribosylation factor-like A1D | Class I |
| At5g10180 | AST68, SULTR2; 1, slufate transporter 2; 1 | Class I |
| At5g42370 | Calcineurin-like metallo-phosphoesterase superfamily protein | Class I |
| At5g26760 | unknown protein; Has 30201 Blast hits to 17322 proteins in 780 species: Archae - 12; Bacteria - | Class I |
| | 1396; Metazoa - 17338; Fungi - 3422; Plants - 5037; Viruses - 0; Other Eukaryotes - 2996 | |
| | (source: NCBI BLink). | |
| At4g17615 | ATCBL1, CBL1, SCABP5, calcineurin B-like protein 1 | Class I |
| At1g29690 | CAD1, MAC/Perforin domain-containing protein | Class I |
| At3g16857 | ARR1, RR1, response regulator 1 | Class I |
| At3g15500 | ANAC055, ATNAC3, NAC055, NAC3, NAC domain containing protein 3 | Class I |
| At5g64650 | Ribosomal protein L17 family protein | Class I |
| At3g13790 | ATBFRUCT1, ATCWINV1, Glycosyl hydrolases family 32 protein | Class I |
| At5g05600 | 2-oxoglutarate (2OG) and Fe(II)-dependent oxygenase superfamily protein | Class I |
| At4g01370 | ATMPK4, MPK4, MAP kinase 4 | Class I |
| At2g41430 | CID1, ERD15, LSR1, dehydration-induced protein (ERD15) | Class I |
| At3g22900 | NRPD7, RNA polymerase Rpb7-like, N-terminal domain | Class I |
| At1g14040 | EXS (ERD1/XPR1/SYG1) family protein | Class I |
| At3g52930 | Aldolase superfamily protein | Class I |
| At2g29080 | ftsh3, FTSH protease 3 | Class I |
| At4g16680 | P-loop containing nucleoside triphosphate hydrolases superfamily protein | Class I |
| At4g39640 | GGT1, gamma-glutamyl transpeptidase 1 | Class I |
| At2g32120 | HSP70T-2, heat-shock protein 70T-2 | Class I |
| At1g23480 | ATCSLA03, ATCSLA3, CSLA03, CSLA03, CSLA3, cellulose synthase-like A3 | Class I |
| At1g15080 | ATLPP2, ATPAP2, LPP2, lipid phosphate phosphatase 2 | Class I |
| At3g13320 | atcax2, CAX2, cation exchanger 2 | Class I |
| At1g43900 | Protein phosphatase 2C family protein | Class I |
| At2g04040 | ATDTX1, TX1, MATE efflux family protein | Class I |
| At3g56800 | acam-3, CAM3, calmodulin 3 | Class I |
| At2g30240 | ATCHX13, CHX13, Cation/hydrogen exchanger family protein | Class I |
| At4g12730 | FLA2, FASCICLIN-like arabinogalactan 2 | Class I |
| At5g53110 | RING/U-box superfamily protein | Class I |
| At5g05790 | Duplicated homeodomain-like superfamily protein | Class I |
| At3g19020 | Leucine-rich repeat (LRR) family protein | Class I |
| At5g17360 | BEST Arabidopsis thaliana protein match is: DNA LIGASE 6 (TAIR: AT1G66730.1); Has | Class I |
| | 1807 Blast hits to 1807 proteins in 277 species: Archae - 0; Bacteria - 0; Metazoa - 736; Fungi - | |
| | 347; Plants - 385; Viruses - 0; Other Eukaryotes - 339 (source: NCBI BLink). | |
| At3g25610 | ATPase E1-E2 type family protein/haloacid dehalogenase-like hydrolase family protein | Class I |
| At1g61890 | MATE efflux family protein | Class I |
| At5g56980 | unknown protein; FUNCTIONS IN: molecular_function unknown; INVOLVED IN: | Class I |
| | biological_process unknown; LOCATED IN: endomembrane system; EXPRESSED IN: 18 | |
| | plant structures; EXPRESSED DURING: 12 growth stages; BEST Arabidopsis thaliana | |
| | protein match is: unknown protein (TAIR: AT4G26130.1); Has 30201 Blast hits to 17322 | |
| | proteins in 780 species: Archae - 12; Bacteria - 1396; Metazoa - 17338; Fungi - 3422; Plants - | |
| | 5037; Viruses - 0; Other Eukaryotes - 2996 (source: NCBI BLink). | |
| At5g07730 | unknown protein; BEST Arabidopsis thaliana protein match is unknown protein | Class I |
| | (TAIR: AT5G61360.1); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At3g59360 | ATUTR6, UTR6, UDP-galactose transporter 6 | Class I |
| At5g44320 | Eukaryotic translation initiation factor 3 subunit 7 (eIF-3) | Class I |
| At4g33666 | unknown protein; FUNCTIONS IN: molecular_function unknown; INVOLVED IN: | Class I |
| | biological_process unknown; LOCATED IN: chloroplast; EXPRESSED IN: 19 plant | |
| | structures; EXPRESSED DURING: 11 growth stages; Has 30201 Blast hits to 17322 proteins | |
| | in 780 species: Archae - 12; Bacteria - 1396; Metazoa - 17338; Fungi - 3422; Plants - 5037; | |
| | Viruses - 0; Other Eukaryotes - 2996 (source: NCBI BLink). | |
| At5g42050 | DCD (Development and Cell Death) domain protein | Class I |
| At4g19210 | ATRLI2, RLI2, RNAse 1 inhibitor protein 2 | Class I |
| At5g43450 | 2-oxoglutarate (2OG) and Fe(II)-dependent oxygenase superfamily protein | Class I |
| At2g07050 | CAS1, cycloartenol synthase 1 | Class I |
| At1g60190 | ARM repeat superfamily protein | Class I |
| At1g68840 | EDF2, RAP2.8, RAV2, TEM2, related to ABI3/VP1 2 | Class I |
| At4g36640 | Sec14p-like phosphatidylinositol transfer family protein | Class I |
| At3g53480 | ABCG37, ATPDR9, PDR9, PIS1, pleiotropic drug resistance 9 | Class I |
| At2g31690 | alpha/beta-Hydrolases superfamily protein | Class I |
| At5g61910 | DCD (Development and Cell Death) domain protein | Class I |
| At1g35140 | EXL7, PHI-1, Phosphate-responsive 1 family protein | Class I |
| At3g04730 | IAA16, indoleacetic acid-induced protein 16 | Class I |
| At2g45400 | BEN1, NAD(P)-binding Rossmann-fold superfamily protein | Class I |
| At1g30700 | FAD-binding Berberine family protein | Class I |
| At4g00170 | Plant VAMP (vesicle-associated membrane protein) family protein | Class I |
| At4g39090 | RD19, RD19A, Papain family cysteine protease | Class I |
| At1g05330 | unknown protein; Has 6 Blast hits to 6 proteins in 2 species: Archae - 0; Bacteria - 0; Metazoa - | Class I |
| | 0; Fungi - 0; Plants - 6; Viruses - 0; Other Eukaryotes - 0 (source: NCBI BLink). | |
| At5g01750 | Protein of unknown function (DUF567) | Class I |
| At3g10985 | ATWI-12, SAG20, WI12, senescence associated gene 20 | Class I |
| At5g10690 | pentatricopeptide (PPR) repeat-containing protein/CBS domain-containing protein | Class I |
| At3g17390 | MAT4, MTO3, SAMS3, S-adenosylmethionine synthetase family protein | Class I |
| At2g18690 | unknown protein; FUNCTIONS IN: molecular_function unknown; INVOLVED IN: | Class I |
| | biological_process unknown; LOCATED IN: membrane; EXPRESSED IN: 17 plant | |
| | structures; EXPRESSED DURING: 9 growth stages; CONTAINS InterPro DOMAIN/s: | |
| | Protein of unknown function DUF975 (InterPro: IPR010380); BEST Arabidopsis thaliana | |
| | protein match is: unknown protein (TAIR: AT2G18680.1); Has 213 Blast hits to 211 proteins | |
| | in 20 species: Archae - 0; Bacteria - 8; Metazoa - 0; Fungi - 0; Plants - 205; Viruses - 0; Other | |
| | Eukaryotes - 0 (source: NCBI BLink). | |
| At3g52800 | A20/AN1-like zinc finger family protein | Class I |
| At2g38480 | Uncharacterised protein family (UPF0497) | Class I |
| At5g52750 | Heavy metal transport/detoxification superfamily protein | Class I |
| At2g18190 | P-loop containing nucleoside triphosphate hydrolases superfamily protein | Class I |
| At5g52400 | CYP715A1, cytochrome P450, family 715, subfamily A, polypeptide 1 | Class I |
| At1g11670 | MATE efflux family protein | Class I |
| At4g25570 | ACYB-2, Cytochrome b561/ferric reductase transmembrane protein family | Class I |
| At4g34160 | CYCD3, CYCD3; 1, CYCLIN D3; 1 | Class I |
| At3g22370 | AOX1A, ATAOX1A, alternative oxidase 1A | Class I |
| At1g01550 | BPS1, Protein of unknown function (DUF793) | Class I |
| At3g23250 | ATMYB15, ATY19, MYB15, myb domain protein 15 | Class I |
| At3g53610 | ATRAB8, AtRab8B, AtRABE1a, RAB8, RAB GTPase homolog 8 | Class I |
| At5g45110 | ATNPR3, NPR3, NPR1-like protein 3 | Class I |
| At5g45140 | NRPC2, nuclear RNA polymerase C2 | Class I |
| At3g50980 | XERO1, dehydrin xero 1 | Class I |
| At5g58060 | ATGP1, ATYKT61, YKT61, SNARE-like superfamily protein | Class I |
| At1g79990 | structural molecules | Class I |
| At5g03210 | unknown protein; FUNCTIONS IN: molecular_function unknown; INVOLVED IN: | Class I |
| | biological_process unknown; LOCATED IN: endomembrane system; EXPRESSED IN: 11 | |
| | plant structures; EXPRESSED DURING: 7 growth stages; Has 6 Blast hits to 6 proteins in 2 | |
| | species: Archae - 0; Bacteria - 0; Metazoa - 0; Fungi - 0; Plants - 6; Viruses - 0; Other | |
| | Eukaryotes - 0 (source: NCBI BLink). | |
| At5g57550 | XTH25, XTR3, xyloglucan endotransglucosylase/hydrolase 25 | Class I |
| At1g61360 | S-locus lectin protein kinase family protein | Class I |
| At3g19240 | Vacuolar import/degradation, Vid27-related protein | Class I |
| At5g66060 | 2-oxoglutarate (2OG) and Fe(II)-dependent oxygenase superfamily protein | Class I |
| At4g04500 | CRK37, cysteine-rich RLK (RECEPTOR-like protein kinase) 37 | Class I |
| At1g32070 | ATNSI, NSI, nuclear shuttle interacting | Class I |
| At5g49220 | Protein of unknown function (DUF789) | Class I |
| At2g04050 | MATE efflux family protein | Class I |
| At1g09070 | (AT)SRC2, SRC2, soybean gene regulated by cold-2 | Class I |
| At5g55780 | Cysteine/Histidine-rich C1 domain family protein | Class I |
| At5g06290 | 2-Cys Prx B, 2CPB, 2-cysteine peroxiredoxin B | Class I |
| At1g12960 | Ribosomal protein L18e/L15 superfamily protein | Class I |
| At3g46620 | zinc finger (C3HC4-type RING finger) family protein | Class I |
| At3g55640 | Mitochondrial substrate carrier family protein | Class I |
| At5g01960 | RING/U-box superfamily protein | Class I |
| At1g35910 | Haloacid dehalogenase-like hydrolase (HAD) superfamily protein | Class I |
| At1g29680 | Protein of unknown function (DUF1264) | Class I |
| At1g14530 | THH1, Protein of unknown function (DUF1084) | Class I |
| At5g06320 | NHL3, NDR1/HIN1-like 3 | Class I |
| At1g05680 | UGT74E2, Uridine diphosphate glycosyltransferase 74E2 | Class I |
| At4g27270 | Quinone reductase family protein | Class I |
| At3g50970 | LTI30, XERO2, dehydrin family protein | Class I |
| At5g64240 | AtMC3, MC3, metacaspase 3 | Class I |
| At3g02040 | SRG3, senescence-related gene 3 | Class I |
| At4g05320 | UBQ10, polyubiquitin 10 | Class I |
| At3g16860 | COBL8, COBRA-like protein 8 precursor | Class I |
| At5g04750 | F1F0-ATPase inhibitor protein, putative | Class I |
| At4g36500 | unknown protein; FUNCTIONS IN: molecular_function unknown; INVOLVED IN: | Class I |
| | biological_process unknown; LOCATED IN: mitochondrion; EXPRESSED IN: 22 plant | |
| | structures; EXPRESSED DURING: 13 growth stages; BEST Arabidopsis thaliana protein | |
| | match is: unknown protein (TAIR: AT2G18210.1); Has 50 Blast hits to 50 proteins in 7 | |
| | species: Archae - 0; Bacteria - 0; Metazoa - 0; Fungi - 0; Plants - 50; Viruses - 0; Other | |
| | Eukaryotes - 0 (source: NCBI BLink). | |
| At5g17460 | unknown protein; FUNCTIONS IN: molecular_function unknown; INVOLVED IN: response | Class I |
| | to salt stress; LOCATED IN: mitochondrion; Has 30201 Blast hits to 17322 proteins in 780 | |
| | species: Archae - 12; Bacteria - 1396; Metazoa - 17338; Fungi - 3422; Plants - 5037; Viruses - | |
| | 0; Other Eukaryotes - 2996 (source: NCBI BLink). | |
| At3g49530 | ANAC062, NAC062, NTL6, NAC domain containing protein 62 | Class I |
| At1g22080 | Cysteine proteinases superfamily protein | Class I |
| At4g37260 | ATMYB73, MYB73, myb domain protein 73 | Class I |
| At5g02240 | NAD(P)-binding Rossmann-fold superfamily protein | Class I |
| At1g01720 | ANAC002, ATAF1, NAC (No Apical Meristem) domain transcriptional regulator superfamily | Class I |
| | protein | |
| At5g13470 | unknown protein; Has 1807 Blast hits to 1807 proteins in 277 species: Archae - 0; Bacteria - 0; | Class I |
| | Metazoa - 736; Fungi - 347; Plants - 385; Viruses - 0; Other Eukaryotes - 339 (source: NCBI | |
| | BLink). | |
| At1g59870 | ABCG36, ATABCG36, ATPDR8, PDR8, PEN3, ABC-2 and Plant PDR ABC-type transporter | Class I |
| | family protein | |
| At3g52450 | PUB22, plant U-box 22 | Class I |
| At1g49520 | SWIB complex BAF60b domain-containing protein | Class I |
| At1g78290 | SNRK2-8, SNRK2.8, SRK2C, Protein kinase superfamily protein | Class I |
| At3g63380 | ATPase E1-E2 type family protein/haloacid dehalogenase-like hydrolase family protein | Class I |
| At5g25930 | Protein kinase family protein with leucine-rich repeat domain | Class I |
| At4g24580 | REN1, Rho GTPase activation protein (RhoGAP) with PH domain | Class I |
| At1g80850 | DNA glycosylase superfamily protein | Class I |
| At5g37500 | GORK, gated outwardly-rectifying K+ channel | Class I |
| At4g21850 | ATMSRB9, MSRB9, methionine sulfoxide reductase B9 | Class I |
| At3g09440 | Heat shock protein 70 (Hsp 70) family protein | Class I |
| At3g14940 | ATPPC3, PPC3, phosphoenolpyruvate carboxylase 3 | Class I |
| At2g27090 | Protein of unknown function (DUF630 and DUF632) | Class I |
| At3g45730 | unknown protein; Has 3 Blast hits to 3 proteins in 1 species: Archae - 0; Bacteria - 0; Metazoa - | Class I |
| | 0; Fungi - 0; Plants - 3; Viruses - 0; Other Eukaryotes - 0 (source: NCBI BLink). | |
| At5g63780 | SHA1, RING/FYVE/PHD zinc finger superfamily protein | Class I |
| At3g08590 | Phosphoglycerate mutase, 2,3-bisphosphoglycerate-independent | Class I |
| At2g40000 | ATHSPRO2, HSPRO2, ortholog of sugar beet HS1 PRO-1 2 | Class I |
| At5g66055 | AKRP, EMB16, EMB2036, ankyrin repeat protein | Class I |
| At1g17870 | ATEGY3, EGY3, ethylene-dependent gravitropism-deficient and yellow-green-like 3 | Class I |
| At1g69220 | SIK1, Protein kinase superfamily protein | Class I |
| At5g20240 | PI, K-box region and MADS-box transcription factor family protein | Class I |
| At1g68760 | ATNUDT1, ATNUDX1, NUDX1, NUDX1, nudix hydrolase 1 | Class I |
| At1g20440 | AtCOR47, COR47, RD17, cold-regulated 47 | Class I |
| At1g19180 | JAZ1, TIFY10A, jasmonate-zim-domain protein 1 | Class I |
| At5g52410 | CONTAINS InterPro DOMAIN/s: S-layer homology domain (InterPro: IPR001119); BEST | Class I |
| | Arabidopsis thaliana protein match is: unknown protein (TAIR: AT5G23890.1); Has 30201 | |
| | Blast hits to 17322 proteins in 780 species: Archae - 12; Bacteria - 1396; Metazoa - 17338; | |
| | Fungi - 3422; Plants - 5037; Viruses - 0; Other Eukaryotes - 2996 (source: NCBI BLink). | |
| At5g39580 | Peroxidase superfamily protein | Class I |
| At5g15980 | Pentatricopeptide repeat (PPR) superfamily protein | Class I |
| At3g24050 | GATA1, GATA transcription factor 1 | Class I |
| At1g61870 | PPR336, pentatricopeptide repeat 336 | Class I |
| At5g10710 | INVOLVED IN: chromosome segregation, cell division; LOCATED IN: chromosome, | Class I |
| | centromeric region, nucleus; EXPRESSED IN: 23 plant structures; EXPRESSED DURING: | |
| | 13 growth stages; CONTAINS InterPro DOMAIN/s: Centromere protein Cenp-O | |
| | (InterPro: IPR018464); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At1g50750 | Plant mobile domain protein family | Class I |
| At5g05420 | FKBP-like peptidyl-prolyl cis-trans isomerase family protein | Class I |
| At1g09080 | BIP3, Heat shock protein 70 (Hsp 70) family protein | Class I |
| At1g58210 | EMB1674, kinase interacting family protein | Class I |
| At5g02020 | Encodes a protein involved in salt tolerance, names SIS (Salt Induced Serine rich). | Class I |
| At2g39190 | ATATH8, Protein kinase superfamily protein | Class I |
| At1g62790 | Bifunctional inhibitor/lipid-transfer protein/seed storage 2S albumin superfamily protein | Class I |
| At4g26040 | unknown protein; Has 2 Blast hits to 2 proteins in 1 species: Archae - 0; Bacteria - 0; Metazoa - | Class I |
| | 0; Fungi - 0; Plants - 2; Viruses - 0; Other Eukaryotes - 0 (source: NCBI BLink). | |
| At3g23460 | S-adenosyl-L-methionine-dependent methyltransferases superfamily protein | Class I |
| At2g36950 | Heavy metal transport/detoxification superfamily protein | Class I |
| At5g04330 | Cytochrome P450 superfamily protein | Class I |
| At2g23320 | WRKY15, WRKY DNA-binding protein 15 | Class I |
| At2g23810 | TET8, tetraspanin8 | Class I |
| At3g03890 | FMN binding | Class I |
| At1g17180 | ATGSTU25, GSTU25, glutathione S-transferase TAU 25 | Class I |
| At1g56660 | unknown protein; Has 665200 Blast hits to 205811 proteins in 4684 species: Archae - 3320; | Class I |
| | Bacteria - 107592; Metazoa - 249086; Fungi - 76753; Plants - 38542; Viruses - 3008; Other | |
| | Eukaryotes - 186899 (source: NCBI BLink). | |
| At4g33670 | NAD(P)-linked oxidoreductase superfamily protein | Class I |
| At1g05340 | unknown protein; FUNCTIONS IN: molecular_function unknown; INVOLVED IN: | Class I |
| | biological_process unknown; LOCATED IN: chloroplast; EXPRESSED IN: 14 plant | |
| | structures; EXPRESSED DURING: 7 growth stages; BEST Arabidopsis thaliana protein | |
| | match is: unknown protein (TAIR: AT2G32210.1); Has 189 Blast hits to 189 proteins in 27 | |
| | species: Archae - 0; Bacteria - 0; Metazoa - 0; Fungi - 21; Plants - 168; Viruses - 0; Other | |
| | Eukaryotes - 0 (source: NCBI BLink). | |
| At3g55440 | ATCTIMC, CYTOTPI, TPI, triosephosphate isomerase | Class I |
| At3g49000 | RNA polymerase III subunit RPC82 family protein | Class I |
| At4g25820 | ATXTH14, XTH14, XTR9, xyloglucan endotransglucosylase/hydrolase 14 | Class I |
| At1g27770 | ACA1, PEA1, autoinhibited Ca2+-ATPase 1 | Class I |
| At5g09990 | PROPEP5, elicitor peptide 5 precursor | Class I |
| At5g10630 | Translation elongation factor EF1A/initiation factor IF2gamma family protein | Class I |
| At4g16830 | Hyaluronan/mRNA binding family | Class I |
| At3g13920 | EIF4A1, RH4, TIF4A1, eukaryotic translation initiation factor 4A1 | Class I |
| At1g25550 | myb-like transcription factor family protein | Class I |
| At5g24650 | Mitochondrial import inner membrane translocase subunit Tim17/Tim22/Tim23 family protein | Class I |
| At3g59350 | Protein kinase superfamily protein | Class I |
| At2g29470 | ATGSTU3, GST21, GSTU3, glutathione 5-transferase tau 3 | Class I |
| At4g33925 | unknown protein; Has 30201 Blast hits to 17322 proteins in 780 species: Archae - 12; Bacteria - | Class I |
| | 1396; Metazoa - 17338; Fungi - 3422; Plants - 5037; Viruses - 0; Other Eukaryotes - 2996 | |
| | (source: NCBI BLink). | |
| At4g25580 | CAP160 protein | Class I |
| At2g03750 | P-loop containing nucleoside triphosphate hydrolases superfamily protein | Class I |
| At1g42990 | ATBZIP60, BZIP60, BZIP60, basic region/leucine zipper motif 60 | Class I |
| At5g36260 | Eukaryotic aspartyl protease family protein | Class I |
| At1g78080 | RAP2.4, related to AP2 4 | Class I |
| At2g37975 | Yos1-like protein | Class I |
| At5g55140 | ribosomal protein L30 family protein | Class I |
| At3g08610 | unknown protein; Has 40 Blast hits to 40 proteins in 15 species: Archae - 0; Bacteria - 0; | Class I |
| | Metazoa - 0; Fungi - 0; Plants - 40; Viruses - 0; Other Eukaryotes - 0 (source: NCBI BLink). | |
| At5g57190 | PSD2, phosphatidylserine decarboxylase 2 | Class I |
| At1g27720 | TAF4, TAF4B, TBP-associated factor 4B | Class I |
| At1g30740 | FAD-binding Berberine family protein | Class I |
| At2g24570 | ATWRKY17, WRKY17, WRKY DNA-binding protein 17 | Class I |
| At2g44790 | UCC2, uclacyanin 2 | Class I |
| At3g49780 | ATPSK3 (FORMER SYMBOL), ATPSK4, PSK4, phytosulfokine 4 precursor | Class I |
| At3g51920 | ATCML9, CAM9, CML9, calmodulin 9 | Class I |
| At5g65660 | hydroxyproline-rich glycoprotein family protein | Class I |
| At3g19030 | unknown protein; FUNCTIONS IN: molecular_function unknown; INVOLVED IN: | Class I |
| | pyridoxine biosynthetic process, homoserine biosynthetic process; LOCATED IN: | |
| | endomembrane system; EXPRESSED IN: 19 plant structures; EXPRESSED DURING: 9 | |
| | growth stages; BEST Arabidopsis thaliana protein match is: unknown protein | |
| | (TAIR: AT1G49500.1); Has 22 Blast hits to 22 proteins in 2 species: Archae - 0; Bacteria - 0; | |
| | Metazoa - 0; Fungi - 0; Plants - 22; Viruses - 0; Other Eukaryotes - 0 (source: NCBI BLink). | |
| At4g11570 | Haloacid dehalogenase-like hydrolase (HAD) superfamily protein | Class I |
| At4g11560 | bromo-adjacent homology (BAH) domain-containing protein | Class I |
| At3g19580 | AZF2, ZF2, zinc-finger protein 2 | Class I |
| At5g44330 | Tetratricopeptide repeat (TPR)-like superfamily protein | Class I |
| At4g21820 | binding; calmodulin binding | Class I |
| At3g08580 | AAC1, ADP/ATP carrier 1 | Class I |
| At5g66460 | Glycosyl hydrolase superfamily protein | Class I |
| At1g74450 | Protein of unknown function (DUF793) | Class I |
| At2g41110 | ATCAL5, CAM2, calmodulin 2 | Class I |
| At4g37270 | ATHMA1, HMA1, heavy metal atpase 1 | Class I |
| At1g29395 | COR413-TM1, COR413IM1, COR414-TM1, COLD REGULATED 314 INNER | Class I |
| | MEMBRANE 1 | |
| At1g20450 | ERD10, LTI29, LTI45, Dehydrin family protein | Class I |
| At1g32640 | ATMYC2, JAI1, JIN1, MYC2, RD22BP1, ZBF1, Basic helix-loop-helix (bHLH) DNA- | Class I |
| | binding family protein | |
| At5g47960 | ATRABA4C, RABA4C, SMG1, RAB GTPase homolog A4C | Class I |
| At3g03810 | EDA30, O-fucosyltransferase family protein | Class I |
| At1g62300 | ATWRKY6, WRKY6, WRKY family transcription factor | Class I |
| At4g13390 | Proline-rich extensin-like family protein | Class I |
| At2g39990 | AteIF3f, EIF2, eIF3F, eukaryotic translation initiation factor 2 | Class I |
| At5g59450 | GRAS family transcription factor | Class I |
| At5g01380 | Homeodomain-like superfamily protein | Class I |
| At4g37370 | CYP81D8, cytochrome P450, family 81, subfamily D, polypeptide 8 | Class I |
| At1g13210 | ACA.1, autoinhibited Ca2+/ATPase II | Class I |
| At2g41620 | Nucleoporin interacting component (Nup93/Nic96-like) family protein | Class I |
| At2g41740 | ATVLN2, VLN2, villin 2 | Class I |
| At5g18475 | Pentatricopeptide repeat (PPR) superfamily protein | Class I |
| At2g17840 | ERD7, Senescence/dehydration-associated protein-related | Class I |
| At2g25490 | EBF1, FBL6, EIN3-binding F box protein 1 | Class I |
| At4g20840 | FAD-binding Berberine family protein | Class I |
| At1g53830 | ATPME2, PME2, pectin methylesterase 2 | Class I |
| At5g59830 | unknown protein; BEST Arabidopsis thaliana protein match is: unknown protein | Class I |
| | (TAIR: AT5G13660.2); Has 174 Blast hits to 139 proteins in 16 species: Archae - 0; Bacteria - | |
| | 0; Metazoa - 0; Fungi - 0; Plants - 172; Viruses - 0; Other Eukaryotes - 2 (source: NCBI | |
| | BLink). | |
| At1g01060 | LHY, LHY1, Homeodomain-like superfamily protein | Class I |
| At1g31820 | Amino acid permease family protein | Class I |
| At1g80010 | FRS8, FAR1-related sequence 8 | Class I |
| At2g45810 | DEA(D/H)-box RNA helicase family protein | Class I |
| At1g55450 | S-adenosyl-L-methionine-dependent methyltransferases superfamily protein | Class I |
| At1g21850 | sks8, SKU5 similar 8 | Class I |
| At5g50900 | ARM repeat superfamily protein | Class I |
| At3g56880 | VQ motif-containing protein | Class I |
| At1g76180 | ERD14, Dehydrin family protein | Class I |
| At4g25810 | XTH23, XTR6, xyloglucan endotransglycosylase 6 | Class I |
| At3g24170 | ATGR1, GR1, glutathione-disulfide reductase | Class I |
| At5g47210 | Hyaluronan/mRNA binding family | Class I |
| At5g07450 | CYCP4; 3, cyclin p4; 3 | Class I |
| At2g39670 | Radical SAM superfamily protein | Class I |
| At1g56670 | GDSL-like Lipase/Acylhydrolase superfamily protein | Class I |
| At5g08230 | Tudor/PWWP/MBT domain-containing protein | Class I |
| At3g24560 | RSY3, Adenine nucleotide alpha hydrolases-like superfamily protein | Class I |
| At1g17860 | Kunitz family trypsin and protease inhibitor protein | Class I |
| At3g57460 | catalytics; metal ion binding | Class I |
| At2g20570 | ATGLK1, GLK1, GPRI1, GBF's pro-rich region-interacting factor 1 | Class I |
| At3g21500 | DXPS1, 1-deoxy-D-xylulose 5-phosphate synthase 1 | Class I |
| At3g25650 | ASK15, SK15, SKP1-like 15 | Class I |
| At5g46780 | VQ motif-containing protein | Class I |
| At5g43440 | 2-oxoglutarate (2OG) and Fe(II)-dependent oxygenase superfamily protein | Class I |
| At3g50910 | unknown protein; BEST Arabidopsis thaliana protein match is: unknown protein | Class I |
| | (TAIR: AT5G66480.1); Has 76 Blast hits to 75 proteins in 28 species: Archae - 0; Bacteria - | |
| | 10; Metazoa - 7; Fungi - 2; Plants - 49; Viruses - 0; Other Eukaryotes - 8 (source: NCBI | |
| | BLink). | |
| At4g26180 | Mitochondrial substrate carrier family protein | Class I |
| At3g25250 | AGC2, AGC2-1, AtOXI1, OXI1, AGC (cAMP-dependent, cGMP-dependent and protein | Class I |
| | kinase C) kinase family protein | |
| At1g59600 | ZCW7, ZCW7 | Class I |
| At2g05720 | Transducin/WD40 repeat-like superfamily protein | Class I |
| At2g43290 | MSS3, Calcium-binding EF-hand family protein | Class I |
| At3g53760 | ATGCP4, GCP4, GAMMA-TUBULIN COMPLEX PROTEIN 4 | Class I |
| At5g11670 | ATNADP-ME2, NADP-ME2, NADP-malic enzyme 2 | Class I |
| At5g07740 | actin binding | Class I |
| At5g27420 | ATL31, CNI1, carbon/nitrogen insensitive 1 | Class I |
| At3g15460 | Ribosomal RNA processing Brix domain protein | Class I |
| At5g47230 | ATERF-5, ATERF5, ERF5, ethylene responsive element binding factor 5 | Class I |
| At3g62410 | CP12, CP12-2, CP12 domain-containing protein 2 | Class I |
| At5g03610 | GDSL-like Lipase/Acylhydrolase superfamily protein | Class I |
| At4g05050 | UBQ11, ubiquitin 11 | Class I |
| At1g22200 | Endoplasmic reticulum vesicle transporter protein | Class I |
| At4g32920 | glycine-rich protein | Class I |
| At5g59820 | RHL41, ZAT12, C2H2-type zinc finger family protein | Class I |
| At5g49030 | OVA2, tRNA synthetase class I (I, L, M and V) family protein | Class I |
| At1g68670 | myb-like transcription factor family protein | Class I |
| At5g26360 | TCP-1/cpn60 chaperonin family protein | Class I |
| At5g24800 | ATBZIP9, BZIP9, BZO2H2, basic leucine zipper 9 | Class I |
| At4g00690 | ULP1B, UB-like protease 1B | Class I |
| At3g06500 | Plant neutral invertase family protein | Class I |
| At1g80930 | MIF4G domain-containing protein/MA3 domain-containing protein | Class I |
| At1g69880 | ATH8, TH8, thioredoxin H-type 8 | Class I |
| At5g24930 | ATCOL4, COL4, CONSTANS-like 4 | Class I |
| At2g46260 | BTB/POZ/Kelch-associated protein | Class I |
| At1g19020 | unknown protein; BEST Arabidopsis thaliana protein match is: unknown protein | Class I |
| | (TAIR: AT3G48180.1); Has 88 Blast hits to 88 proteins in 15 species: Archae - 0; Bacteria - 0; | |
| | Metazoa - 0; Fungi - 0; Plants - 88; Viruses - 0; Other Eukaryotes - 0 (source: NCBI BLink). | |
| At1g68765 | IDA, Putative membrane lipoprotein | Class I |
| At1g56590 | ZIP4, Clathrin adaptor complexes medium subunit family protein | Class I |
| At5g01820 | ATCIPK14, ATSR1, CIPK14, SnRK3.15, SR1, serine/threonine protein kinase 1 | Class I |
| At4g05100 | AtMYB74, MYB74, myb domain protein 74 | Class I |
| At5g58070 | ATTIL, TIL, temperature-induced lipocalin | Class I |
| At5g15090 | ATVDAC3, VDAC3, voltage dependent anion channel 3 | Class I |
| At3g06510 | ATSFR2, SFR2, Glycosyl hydrolase superfamily protein | Class I |
| At2g40140 | ATSZF2, CZF1, SZF2, ZFAR1, zinc finger (CCCH-type) family protein | Class I |
| At3g50960 | PLP3a, phosducin-like protein 3 homolog | Class I |
| At1g17850 | Rhodanese/Cell cycle control phosphatase superfamily protein | Class I |
| At1g28280 | VQ motif-containing protein | Class I |
| At4g36010 | Pathogenesis-related thaumatin superfamily protein | Class I |
| At3g44260 | Polynucleotidyl transferase, ribonuclease H-like superfamily protein | Class I |
| At5g35735 | Auxin-responsive family protein | Class I |
| At1g01725 | unknown protein; FUNCTIONS IN: molecular_function unknown; INVOLVED IN: | Class I |
| | biological_process unknown; LOCATED IN: endomembrane system; EXPRESSED IN: 23 | |
| | plant structures; EXPRESSED DURING: 14 growth stages; BEST Arabidopsis thaliana | |
| | protein match is: unknown protein (TAIR: AT4G00530.1); Has 20 Blast hits to 20 proteins in 7 | |
| | species: Archae - 0; Bacteria - 0; Metazoa - 0; Fungi - 0; Plants - 20; Viruses - 0; Other | |
| | Eukaryotes - 0 (source: NCBI BLink). | |
| At3g45740 | hydrolase family protein/HAD-superfamily protein | Class I |
| At3g55620 | emb1624, Translation initiation factor IF6 | Class I |
| At5g63790 | ANAC102, NAC102, NAC domain containing protein 102 | Class I |
| At2g34910 | BEST Arabidopsis thaliana protein match is: root hair specific 4 (TAIR: AT1G30850.1); Has | Class I |
| | 43 Blast hits to 43 proteins in 9 species: Archae - 0; Bacteria - 0; Metazoa - 0; Fungi - 0; | |
| | Plants - 43; Viruses - 0; Other Eukaryotes - 0 (source: NCBI BLink). | |
| At5g01510 | RUS5, Protein of unknown function, DUF647 | Class I |
| At2g43130 | ARA-4, ARA4, ATRAB11F, ATRABA5C, RABA5C, P-loop containing nucleoside | Class I |
| | triphosphate hydrolases superfamily protein | |
| At3g22380 | TIC, time for coffee | Class I |
| At1g45145 | ATH5, ATTRX5, LIV1, TRX5, thioredoxin H-type 5 | Class I |
| At1g22070 | TGA3, TGA1A-related gene 3 | Class I |
| At5g14740 | BETA CA2, CA18, CA2, carbonic anhydrase 2 | Class I |
| At2g18240 | Rer1 family protein | Class I |
| At2g46420 | Plant protein 1589 of unknown function | Class I |
| At5g56340 | ATCRT1, RING/U-box superfamily protein | Class I |
| At5g18310 | unknown protein; FUNCTIONS IN: molecular_function unknown; INVOLVED IN: | Class I |
| | biological_process unknown; LOCATED IN: plasma membrane; EXPRESSED IN: 22 plant | |
| | structures; EXPRESSED DURING: 13 growth stages; BEST Arabidopsis thaliana protein | |
| | match is: unknown protein (TAIR: AT5G48500.1); Has 30201 Blast hits to 17322 proteins in | |
| | 780 species: Archae - 12; Bacteria - 1396; Metazoa - 17338; Fungi - 3422; Plants - 5037; | |
| | Viruses - 0; Other Eukaryotes - 2996 (source: NCBI BLink). | |
| At3g28690 | Protein kinase superfamily protein | Class I |
| At3g15210 | ATERF-4, ATERF4, ERF4, RAP2.5, ethylene responsive element binding factor 4 | Class I |
| At1g69760 | unknown protein; BEST Arabidopsis thaliana protein match is: unknown protein | Class I |
| | (TAIR: AT1G26920.1); Has 51 Blast hits to 51 proteins in 15 species: Archae - 0; Bacteria - 2; | |
| | Metazoa - 2; Fungi - 7; Plants - 29; Viruses - 0; Other Eukaryotes - 11 (source: NCBI BLink). | |
| At2g46390 | unknown protein; FUNCTIONS IN: molecular_function unknown; INVOLVED IN: | Class I |
| | biological_process unknown; LOCATED IN: endomembrane system; EXPRESSED IN: 24 | |
| | plant structures; EXPRESSED DURING: 15 growth stages; Has 4 Blast hits to 4 proteins in 2 | |
| | species: Archae - 0; Bacteria - 0; Metazoa - 0; Fungi - 0; Plants - 4; Viruses - 0; Other | |
| | Eukaryotes - 0 (source: NCBI BLink). | |
| At2g17080 | Arabidopsis protein of unknown function (DUF241) | Class I |
| At1g76170 | 2-thiocytidine tRNA biosynthesis protein, TtcA | Class I |
| At5g61890 | Integrase-type DNA-binding superfamily protein | Class I |
| At2g20560 | DNAJ heat shock family protein | Class I |
| At4g30600 | signal recognition particle receptor alpha subunit family protein | Class I |
| At3g19570 | Family of unknown function (DUF566) | Class I |
| At5g11740 | AGP15, ATAGP15, arabinogalactan protein 15 | Class I |
| At1g04530 | Tetratricopeptide repeat (TPR)-like superfamily protein | Class I |
| At2g29490 | ATGSTU1, GST19, GSTU1, glutathione S-transferase TAU 1 | Class I |
| At5g61520 | Major facilitator superfamily protein | Class I |
| At4g02880 | unknown protein; BEST Arabidopsis thaliana protein match is: unknown protein | Class I |
| | (TAIR: AT1G03290.2); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At1g43910 | P-loop containing nucleoside triphosphate hydrolases superfamily protein | Class I |
| At2g30250 | ATWRKY25, WRKY25, WRKY DNA-binding protein 25 | Class I |
| At4g08950 | EXO, Phosphate-responsive 1 family protein | Class I |
| At4g20830 | FAD-binding Berberine family protein | Class I |
| At1g18740 | Protein of unknown function (DUF793) | Class I |
| At3g01560 | Protein of unknown function (DUF1421) | Class I |
| At5g10700 | Peptidyl-tRNA hydrolase II (PTH2) family protein | Class I |
| At2g41410 | Calcium-binding EF-hand family protein | Class I |
| At4g33780 | FUNCTIONS IN: molecular_function unknown; INVOLVED IN: biological_process | Class I |
| | unknown; LOCATED IN: chloroplast; EXPRESSED IN: 24 plant structures; EXPRESSED | |
| | DURING: 15 growth stages; BEST Arabidopsis thaliana protein match is: short hypocotyl in | |
| | white light1 (TAIR: AT1G69935.1); Has 40 Blast hits to 40 proteins in 10 species: Archae - 0; | |
| | Bacteria - 0; Metazoa - 0; Fungi - 0; Plants - 40; Viruses - 0; Other Eukaryotes - 0 (source: | |
| | NCBI BLink). | |
| At3g60130 | BGLU16, beta glucosidase 16 | Class I |
| At1g42560 | ATMLO9, MLO9, Seven transmembrane MLO family protein | Class I |
| At2g35930 | PUB23, plant U-box 23 | Class I |
| At3g04130 | Tetratricopeptide repeat (TPR)-like superfamily protein | Class I |
| At5g49480 | ATCP1, CP1, Ca2+-binding protein 1 | Class I |
| At4g37010 | CEN2, centrin 2 | Class I |
| At3g52810 | ATPAP21, PAP21, purple acid phosphatase 21 | Class I |
| At1g10170 | ATNFXL1, NFXL1, NF-X-like 1 | Class I |
| At2g41000 | Chaperone DnaJ-domain superfamily protein | Class I |
| At1g33590 | Leucine-rich repeat (LRR) family protein | Class I |
| At5g64905 | PROPEP3, elicitor peptide 3 precursor | Class I |
| At5g62530 | ALDH12A1, ATP5CDH, P5CDH, aldehyde dehydrogenase 12A1 | Class I |
| At1g79400 | ATCHX2, CHX2, cation/H+ exchanger 2 | Class I |
| At4g16670 | Plant protein of unknown function (DUF828) with plant pleckstrin homology-like region | Class I |
| At4g27652 | unknown protein; BEST Arabidopsis thaliana protein match is: unknown protein | Class I |
| | (TAIR: AT4G27657.1); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At5g25280 | serine-rich protein-related | Class I |
| At2g03760 | AtSOT1, AtSOT12, ATST1, RAR047, SOT12, ST, ST1, sulphotransferase 12 | Class I |
| At1g01460 | ATPIPK11, PIPK11, Phosphatidylinositol-4-phosphate 5-kinase, core | Class I |
| At4g11670 | Protein of unknown function (DUF810) | Class I |
| At4g27580 | unknown protein; FUNCTIONS IN: molecular_function unknown; INVOLVED IN: | Class I |
| | biological_process unknown; LOCATED IN: mitochondrion, cell wall; EXPRESSED IN: 9 | |
| | plant structures; EXPRESSED DURING: 6 growth stages; Has 30201 Blast hits to 17322 | |
| | proteins in 780 species: Archae - 12; Bacteria - 1396; Metazoa - 17338; Fungi - 3422; Plants - | |
| | 5037; Viruses - 0; Other Eukaryotes - 2996 (source: NCBI BLink). | |
| At3g05310 | MIRO3, MIRO-related GTP-ase 3 | Class I |
| At3g12120 | FAD2, fatty acid desaturase 2 | Class I |
| At4g28460 | unknown protein; FUNCTIONS IN: molecular_function unknown; INVOLVED IN: | Class I |
| | biological_process unknown; LOCATED IN: endomembrane system; EXPRESSED IN: 10 | |
| | plant structures; EXPRESSED DURING: LP.04 four leaves visible, 4 anthesis, petal | |
| | differentiation and expansion stage; Has 8 Blast hits to 8 proteins in 3 species: Archae - 0; | |
| | Bacteria - 0; Metazoa - 0; Fungi - 0; Plants - 8; Viruses - 0; Other Eukaryotes - 0 (source: | |
| | NCBI BLink). | |
| At2g17670 | Tetratricopeptide repeat (TPR)-like superfamily protein | Class I |
| At3g61640 | AGP20, AtAGP20, arabinogalactan protein 20 | Class I |
| At4g18170 | ATWRKY28, WRKY28, WRKY DNA-binding protein 28 | Class I |
| At4g31805 | WRKY family transcription factor | Class I |
| At1g76600 | unknown protein; FUNCTIONS IN: molecular_function unknown; INVOLVED IN: N- | Class I |
| | terminal protein myristoylation; LOCATED IN: nucleolus, nucleus; EXPRESSED IN: 24 | |
| | plant structures; EXPRESSED DURING: 15 growth stages; BEST Arabidopsis thaliana | |
| | protein match is: unknown protein (TAIR: AT1G21010.1); Has 220 Blast hits to 220 proteins | |
| | in 14 species: Archae - 0; Bacteria - 0; Metazoa - 0; Fungi - 0; Plants - 220; Viruses - 0; Other | |
| | Eukaryotes - 0 (source: NCBI BLink). | |
| At1g65510 | unknown protein; FUNCTIONS IN: molecular_function unknown; INVOLVED IN: N- | Class I |
| | terminal protein myristoylation; LOCATED IN: endomembrane system; EXPRESSED IN: 9 | |
| | plant structures; EXPRESSED DURING: LP.06 six leaves visible, LP.04 four leaves visible, 4 | |
| | anthesis, petal differentiation and expansion stage, LP.08 eight leaves visible; BEST | |
| | Arabidopsis thaliana protein match is: unknown protein (TAIR: AT1G65486.1); Has 22 Blast | |
| | hits to 22 proteins in 2 species: Archae - 0; Bacteria - 0; Metazoa - 0; Fungi - 0; Plants - 22; | |
| | Viruses - 0; Other Eukaryotes - 0 (source: NCBI BLink). | |
| At2g46830 | CCA1, circadian clock associated 1 | Class I |
| At4g30440 | GAE1, UDP-D-glucuronate 4-epimerase 1 | Class I |
| At5g65205 | NAD(P)-binding Rossmann-fold superfamily protein | Class I |
| At5g40690 | CONTAINS InterPro DOMAIN/s: EF-Hand 1, calcium-binding site (InterPro: IPR018247); | Class I |
| | BEST Arabidopsis thaliana protein match is: unknown protein (TAIR: AT2G41730.1); Has | |
| | 1807 Blast hits to 1807 proteins in 277 species: Archae - 0; Bacteria - 0; Metazoa - 736; Fungi - | |
| | 347; Plants - 385; Viruses - 0; Other Eukaryotes - 339 (source: NCBI BLink). | |
| At1g74310 | ATHSP101, HOT1, HSP101, heat shock protein 101 | Class I |
| At5g01950 | Leucine-rich repeat protein kinase family protein | Class I |
| At1g56050 | GTP-binding protein-related | Class I |
| At1g22840 | ATCYTC-A, CYTC-1, CYTOCHROME C-1 | Class I |
| At4g19200 | proline-rich family protein | Class I |
| At1g19025 | DNA repair metallo-beta-lactamase family protein | Class I |
| At2g05710 | ACO3, aconitase 3 | Class I |
| At1g08940 | Phosphoglycerate mutase family protein | Class I |
| At2g47000 | ABCB4, ATPGP4, MDR4, PGP4, ATP binding cassette subfamily B4 | Class I |
| At3g27510 | Cysteine/Histidine-rich C1 domain family protein | Class I |
| At4g27280 | Calcium-binding EF-hand family protein | Class I |
| At1g71697 | ATCK1, CK, CK1, choline kinase 1 | Class I |
| At4g21490 | NDB3, NAD(P)H dehydrogenase B3 | Class I |
| At5g47970 | Aldolase-type TIM barrel family protein | Class I |
| At1g18310 | glycosyl hydrolase family 81 protein | Class I |
| At1g71530 | Protein kinase superfamily protein | Class I |
| At2g32150 | Haloacid dehalogenase-like hydrolase (HAD) superfamily protein | Class I |
| At1g59590 | ZCF37, ZCF37 | Class I |
| At1g19770 | ATPUP14, PUP14, purine permease 14 | Class I |
| At4g29790 | unknown protein; BEST Arabidopsis thaliana protein match is unknown protein | Class I |
| | (TAIR: AT2G19390.1); Has 538 Blast hits to 357 proteins in 124 species: Archae - 0; Bacteria - | |
| | 74; Metazoa - 109; Fungi - 58; Plants - 105; Viruses - 2; Other Eukaryotes - 190 (source: | |
| | NCBI BLink). | |
| At1g27760 | ATSAT32, SAT32, interferon-related developmental regulator family protein/IFRD protein | Class I |
| | family | |
| At1g11560 | Oligosaccharyltransferase complex/magnesium transporter family protein | Class I |
| At2g04880 | ATWRKY1, WRKY1, ZAP1, zinc-dependent activator protein-1 | Class I |
| At1g53840 | ATPME1, PME1, pectin methylesterase 1 | Class I |

ClassIIA. BA: bind and activate

| | | |
|---|---|---|
| At1g69490 | ANAC029, ATNAP, NAP, NAC-like, activated by AP3/PI | Class IIA |
| At5g03380 | Heavy metal transport/detoxification superfamily protein | Class IIA |
| At2g23170 | GH3.3, Auxin-responsive GH3 family protein | Class IIA |
| At1g66170 | MMD1, RING/FYVE/PHD zinc finger superfamily protein | Class IIA |
| At4g20860 | FAD-binding Berberine family protein | Class IIA |
| At3g12320 | unknown protein; BEST Arabidopsis thaliana protein match is unknown protein | Class IIA |
| | (TAIR: AT5G06980.4); Has 102 Blast hits to 102 proteins in 16 species: Archae - 0; Bacteria - | |
| | 0; Metazoa - 0; Fungi - 0; Plants - 98; Viruses - 0; Other Eukaryotes - 4 (source: NCBI | |
| | BLink). | |
| At5g06300 | Putative lysine decarboxylase family protein | Class IIA |
| At3g15630 | unknown protein; FUNCTIONS IN: molecular_function unknown; INVOLVED IN: | Class IIA |
| | biological_process unknown; LOCATED IN: chloroplast; EXPRESSED IN: 24 plant | |
| | structures; EXPRESSED DURING: 15 growth stages; BEST Arabidopsis thaliana protein | |
| | match is: unknown protein (TAIR: AT1G52720.1); Has 61 Blast hits to 61 proteins in 13 | |
| | species: Archae - 0; Bacteria - 0; Metazoa - 0; Fungi - 0; Plants - 61; Viruses - 0; Other | |
| | Eukaryotes - 0 (source: NCBI BLink). | |
| At3g19930 | ATSTP4, STP4, sugar transporter 4 | Class IIA |
| At1g43160 | RAP2.6, related to AP2 6 | Class IIA |
| At3g01290 | SPFH/Band 7/PHB domain-containing membrane-associated protein family | Class IIA |
| At2g39200 | ATMLO12, MLO12, Seven transmembrane MLO family protein | Class IIA |
| At3g14990 | Class I glutamine amidotransferase-like superfamily protein | Class IIA |
| At1g69890 | Protein of unknown function (DUF569) | Class IIA |
| At4g15610 | Uncharacterised protein family (UPF0497) | Class IIA |
| At3g15450 | Aluminium induced protein with YGL and LRDR motifs | Class IIA |
| At1g62570 | FMO GS-OX4, flavin-monooxygenase glucosinolate S-oxygenase 4 | Class IIA |
| At1g29400 | AML5, ML5, MEI2-like protein 5 | Class IIA |
| At1g32930 | Galactosyltransferase family protein | Class IIA |
| At5g67420 | ASL39, LBD37, LOB domain-containing protein 37 | Class IIA |
| At5g64120 | Peroxidase superfamily protein | Class IIA |
| At3g30775 | AT-POX, ATPDH, ATPOX, ERD5, PRO1, PRODH, Methylenetetrahydrofolate reductase | Class IIA |
| | family protein | |
| At1g22830 | Tetratricopeptide repeat (TPR)-like superfamily protein | Class IIA |
| At1g22190 | Integrase-type DNA-binding superfamily protein | Class IIA |
| At2g22870 | EMB2001, P-loop containing nucleoside triphosphate hydrolases superfamily protein | Class IIA |
| At5g11090 | serine-rich protein-related | Class IIA |
| At5g07440 | GDH2, glutamate dehydrogenase 2 | Class IIA |
| At5g67310 | CYP81G1, cytochrome P450, family 81, subfamily G, polypeptide 1 | Class IIA |
| At1g68440 | unknown protein; BEST Arabidopsis thaliana protein match is unknown protein | Class IIA |
| | (TAIR: AT1G25400.2); Has 86 Blast hits to 86 proteins in 29 species: Archae - 0; Bacteria - | |
| | 6; Metazoa - 27; Fungi - 11; Plants - 24; Viruses - 0; Other Eukaryotes - 18 (source: NCBI | |
| | BLink). | |
| At1g15040 | Class I glutamine amidotransferase-like superfamily protein | Class IIA |
| At5g43580 | Serine protease inhibitor, potato inhibitor I-type family protein | Class IIA |
| At3g49790 | Carbohydrate-binding protein | Class IIA |
| At5g52050 | MATE efflux family protein | Class IIA |
| At5g12340 | unknown protein; BEST Arabidopsis thaliana protein match is unknown protein | Class IIA |
| | (TAIR: AT1G28190.1); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At4g27657 | unknown protein; FUNCTIONS IN: molecular_function unknown; INVOLVED IN: | Class IIA |
| | biological_process unknown; LOCATED IN: endomembrane system; EXPRESSED IN: 15 | |
| | plant structures; EXPRESSED DURING: 9 growth stages; BEST Arabidopsis thaliana | |
| | protein match is: unknown protein (TAIR: AT5G54145.1); Has 30201 Blast hits to 17322 | |
| | proteins in 780 species: Archae - 12; Bacteria - 1396; Metazoa - 17338; Fungi - 3422; Plants - | |
| | 5037; Viruses - 0; Other Eukaryotes - 2996 (source: NCBI BLink). | |
| At5g02810 | APRR7, PRR7, pseudo-response regulator 7 | Class IIA |
| At3g45970 | ATEXLA1, ATEXPL1, ATHEXP BETA 2.1, EXLA1, EXPL1, expansin-like A1 | Class IIA |
| At4g20870 | ATFAH2, FAH2, fatty acid hydroxylase 2 | Class IIA |
| At1g64670 | BDG1, alpha/beta-Hydrolases superfamily protein | Class IIA |
| At3g60140 | BGLU30, DIN2, SRG2, Glycosyl hydrolase superfamily protein | Class IIA |
| At1g64660 | ATMGL, MGL, methionine gamma-lyase | Class IIA |
| At5g67300 | ATMYB44, ATMYBR1, MYB44, MYBR1, myb domain protein r1 | Class IIA |
| At5g20150 | ATSPX1, SPX1, SPX domain gene 1 | Class IIA |
| At4g36040 | Chaperone DnaJ-domain superfamily protein | Class IIA |
| At5g40780 | LHT1, lysine histidine transporter 1 | Class IIA |
| At1g80380 | P-loop containing nucleoside triphosphate hydrolases superfamily protein | Class IIA |
| At1g27100 | Actin cross-linking protein | Class IIA |
| At3g15620 | UVR3, DNA photolyase family protein | Class IIA |
| At5g01600 | ATFER1, FER1, ferretin 1 | Class IIA |
| At3g52710 | unknown protein; FUNCTIONS IN: molecular_function unknown; INVOLVED IN: | Class IIA |
| | biological_process unknown; LOCATED IN: plasma membrane; EXPRESSED IN: 19 plant | |
| | structures; EXPRESSED DURING: 9 growth stages; BEST Arabidopsis thaliana protein | |
| | match is: unknown protein (TAIR: AT2G36220.1); Has 64 Blast hits to 64 proteins in 10 | |
| | species: Archae - 0; Bacteria - 0; Metazoa - 0; Fungi - 0; Plants - 64; Viruses - 0; Other | |
| | Eukaryotes - 0 (source: NCBI BLink). | |
| At3g04070 | anac047, NAC047, NAC domain containing protein 47 | Class IIA |
| At4g37590 | NPY5, Phototropic-responsive NPH3 family protein | Class IIA |
| At5g45630 | Protein of unknown function, DUF584 | Class IIA |

ClassIIB. BR: bind and repress

| | | |
|---|---|---|
| At3g50900 | unknown protein; BEST Arabidopsis thaliana protein match is unknown protein | Class IIB |
| | (TAIR: AT5G66490.1); Has 45 Blast hits to 45 proteins in 7 species: Archae - 0; Bacteria - 0; | |
| | Metazoa - 0; Fungi - 0; Plants - 45; Viruses - 0; Other Eukaryotes - 0 (source: NCBI BLink). | |
| At5g57500 | Galactosyltransferase family protein | Class IIB |
| At1g19190 | alpha/beta-Hydrolases superfamily protein | Class IIB |
| At2g25735 | unknown protein; Has 31 Blast hits to 31 proteins in 9 species: Archae - 0; Bacteria - 0; | Class IIB |
| | Metazoa - 0; Fungi - 0; Plants - 31; Viruses - 0; Other Eukaryotes - 0 (source: NCBI BLink). | |
| At1g56060 | unknown protein; BEST Arabidopsis thaliana protein match is unknown protein | Class IIB |
| | (TAIR: AT2G32210.1); Has 180 Blast hits to 180 proteins in 22 species: Archae - 0; Bacteria - | |
| | 0; Metazoa - 0; Fungi - 10; Plants - 170; Viruses - 0; Other Eukaryotes - 0 (source: NCBI | |
| | BLink). | |
| At4g08850 | Leucine-rich repeat receptor-like protein kinase family protein | Class IIB |
| At5g08240 | unknown protein; BEST Arabidopsis thaliana protein match is unknown protein | Class IIB |
| | (TAIR: AT5G23160.1); Has 69 Blast hits to 69 proteins in 10 species: Archae - 0; Bacteria - | |
| | 1; Metazoa - 0; Fungi - 0; Plants - 68; Viruses - 0; Other Eukaryotes - 0 (source: NCBI | |
| | BLink). | |
| At3g21070 | ATNADK-1, NADK1, NAD kinase 1 | Class IIB |
| At4g37910 | mtHsc70-1, mitochondrial heat shock protein 70-1 | Class IIB |
| At4g12720 | AtNUDT7, ATNUDX7, GFG1, NUDT7, MutT/nudix family protein | Class IIB |
| At3g02880 | Leucine-rich repeat protein kinase family protein | Class IIB |
| At3g06490 | AtMYB108, BOS1, MYB108, myb domain protein 108 | Class IIB |
| At1g18210 | Calcium-binding EF-hand family protein | Class IIB |
| At5g26030 | ATFC-I, FC-I, FC1, ferrochelatase 1 | Class IIB |
| At3g55630 | ATDFD, DFD, DHFS-FPGS homolog D | Class IIB |
| At4g24390 | RNI-like superfamily protein | Class IIB |
| At2g41730 | unknown protein; BEST Arabidopsis thaliana protein match is unknown protein | Class IIB |
| | (TAIR: AT5G24640.1); Has 25 Blast hits to 25 proteins in 5 species: Archae - 0; Bacteria - 0; | |
| | Metazoa - 0; Fungi - 0; Plants - 25; Viruses - 0; Other Eukaryotes - 0 (source: NCBI BLink). | |
| At5g41810 | unknown protein; BEST Arabidopsis thaliana protein match is unknown protein | Class IIB |
| | (TAIR: AT1G64340.1); Has 876 Blast hits to 690 proteins in 132 species: Archae - 0; | |
| | Bacteria - 38; Metazoa - 180; Fungi - 112; Plants - 59; Viruses - 2; Other Eukaryotes - 485 | |
| | (source: NCBI BLink). | |
| At5g02230 | Haloacid dehalogenase-like hydrolase (HAD) superfamily protein | Class IIB |
| At1g16670 | Protein kinase superfamily protein | Class IIB |
| At3g04120 | GAPC, GAPC-1, GAPC1, glyceraldehyde-3-phosphate dehydrogenase C subunit 1 | Class IIB |
| At2g32220 | Ribosomal L27e protein family | Class IIB |
| At5g37770 | CML24, TCH2, EF hand calcium-binding protein family | Class IIB |
| At2g38470 | ATWRKY33, WRKY33, WRKY DNA-binding protein 33 | Class IIB |
| At4g30290 | ATXTH19, XTH19, xyloglucan endotransglucosylase/hydrolase 19 | Class IIB |
| At5g39670 | Calcium-binding EF-hand family protein | Class IIB |
| At1g08510 | FATB, fatty acyl-ACP thioesterases B | Class IIB |
| At3g57450 | unknown protein; Has 65 Blast hits to 65 proteins in 11 species: Archae - 0; Bacteria - 0; | Class IIB |
| | Metazoa - 0; Fungi - 0; Plants - 65; Viruses - 0; Other Eukaryotes - 0 (source: NCBI BLink). | |
| At2g35980 | ATNHL10, NHL10, YLS9, Late embryogenesis abundant (LEA) hydroxyproline-rich | Class IIB |
| | glycoprotein family | |
| At3g24550 | ATPERK1, PERK1, proline extensin-like receptor kinase 1 | Class IIB |
| At1g80820 | ATCCR2, CCR2, cinnamoyl coa reductase | Class IIB |
| At4g34150 | Calcium-dependent lipid-binding (CaLB domain) family protein | Class IIB |
| At5g01540 | LECRKA4.1, lectin receptor kinase a4.1 | Class IIB |
| At1g14540 | Peroxidase superfamily protein | Class IIB |
| At2g41630 | TFIIB, transcription factor IIB | Class IIB |
| At2g38830 | Ubiquitin-conjugating enzyme/RWD-like protein | Class IIB |
| At3g54150 | S-adenosyl-L-methionine-dependent methyltransferases superfamily protein | Class IIB |
| At4g11350 | Protein of unknown function (DUF604) | Class IIB |
| At4g37900 | Protein of unknown function (duplicated DUF1399) | Class IIB |
| At4g30210 | AR2, ATR2, P450 reductase 2 | Class IIB |
| At4g02380 | AtLEA5, SAG21, senescence-associated gene 21 | Class IIB |
| At1g73510 | unknown protein; Has 7 Blast hits to 7 proteins in 2 species: Archae - 0; Bacteria - 0; | Class IIB |
| | Metazoa - 0; Fungi - 0; Plants - 7; Viruses - 0; Other Eukaryotes - 0 (source: NCBI BLink). | |
| At2g41890 | curculin-like (mannose-binding) lectin family protein/PAN domain-containing protein | Class IIB |
| At1g14550 | Peroxidase superfamily protein | Class IIB |
| At4g30280 | ATXTH18, XTH18, xyloglucan endotransglucosylase/hydrolase 18 | Class IIB |
| At5g39680 | EMB2744, Pentatricopeptide repeat (PPR) superfamily protein | Class IIB |
| At4g39260 | ATGRP8, CCR1, GR-RBP8, GRP8, cold, circadian rhythm, and RNA binding 1 | Class IIB |
| At4g38420 | sks9, SKU5 similar 9 | Class IIB |
| At2g46140 | Late embryogenesis abundant protein | Class IIB |
| At1g78340 | ATGSTU22, GSTU22, glutathione S-transferase TAU 22 | Class IIB |
| At2g39660 | BIK1, botrytis-induced kinase1 | Class IIB |
| At4g18880 | AT-HSFA4A, HSF A4A, heat shock transcription factor A4A | Class IIB |
| At4g40040 | Histone superfamily protein | Class IIB |
| At4g11360 | RHA1B, RING-H2 finger A1B | Class IIB |
| At4g30530 | Class I glutamine amidotransferase-like superfamily protein | Class IIB |
| At1g30370 | alpha/beta-Hydrolases superfamily protein | Class IIB |
| At4g40030 | Histone superfamily protein | Class IIB |
| At5g47910 | ATRBOHD, RBOHD, respiratory burst oxidase homologue D | Class IIB |
| At5g64310 | AGP1, ATAGP1, arabinogalactan protein 1 | Class IIB |
| At5g42830 | HXXXD-type acyl-transferase family protein | Class IIB |
| At1g73010 | ATPS2, PS2, phosphate starvation-induced gene 2 | Class IIB |
| At5g19240 | Glycoprotein membrane precursor GPI-anchored | Class IIB |
| At1g06760 | winged-helix DNA-binding transcription factor family protein | Class IIB |
| At2g22500 | ATPUMP5, DIC1, UCP5, uncoupling protein 5 | Class IIB |
| At4g32020 | unknown protein; FUNCTIONS IN: molecular_function unknown; INVOLVED IN: | Class IIB |
| | biological_process unknown; LOCATED IN: chloroplast; EXPRESSED IN: 24 plant | |
| | structures; EXPRESSED DURING: 15 growth stages; BEST Arabidopsis thaliana protein | |
| | match is: unknown protein (TAIR: AT2G25250.1); Has 65 Blast hits to 65 proteins in 19 | |
| | species: Archae - 0; Bacteria - 0; Metazoa - 3; Fungi - 8; Plants - 54; Viruses - 0; Other | |
| | Eukaryotes - 0 (source: NCBI BLink). | |
| At2g17660 | RPM1-interacting protein 4 (RIN4) family protein | Class IIB |
| At2g22470 | AGP2, ATAGP2, arabinogalactan protein 2 | Class IIB |

ClassIIIA. NA: No binding but activation

| | | |
|---|---|---|
| At3g15440 | BEST Arabidopsis thaliana protein match is: RING/U-box superfamily protein | ClassIIIA |
| | (TAIR: AT3G15740.1); Has 12 Blast hits to 12 proteins in 2 species: Archae - 0; Bacteria - 0; | |
| | Metazoa - 0; Fungi - 0; Plants - 12; Viruses - 0; Other Eukaryotes - 0 (source: NCBI BLink). | |
| At4g15280 | UGT71B5, UDP-glucosyl transferase 71B5 | ClassIIIA |
| At3g27690 | LHCB2, LHCB2.3, LHCB2.4, photosystem II light harvesting complex gene 2.3 | ClassIIIA |
| At5g67450 | AZF1, ZF1, zinc-finger protein 1 | ClassIIIA |
| At1g18460 | alpha/beta-Hydrolases superfamily protein | ClassIIIA |
| At1g03600 | PSB27, photosystem II family protein | ClassIIIA |
| At5g44380 | FAD-binding Berberine family protein | ClassIIIA |
| At3g24310 | ATMYB71, MYB305, myb domain protein 305 | ClassIIIA |
| At3g14780 | CONTAINS InterPro DOMAIN/s: Transposase, Ptta/En/Spm, plant (InterPro: IPR004252); | ClassIIIA |
| | BEST Arabidopsis thaliana protein match is: glucan synthase-like 4 (TAIR: AT3G14570.2); | |
| | Has 315 Blast hits to 313 proteins in 50 species: Archae - 2; Bacteria - 16; Metazoa - 11; | |
| | Fungi - 7; Plants - 181; Viruses - 2; Other Eukaryotes - 96 (source: NCBI BLink). | |
| At5g65110 | ACX2, ATACX2, acyl-CoA oxidase 2 | ClassIIIA |
| At1g23870 | ATTPS9, TPS9, TPS9, trehalose-phosphatase/synthase 9 | ClassIIIA |
| At1g08720 | ATEDR1, EDR1, Protein kinase superfamily protein | ClassIIIA |
| At3g03170 | unknown protein; BEST Arabidopsis thaliana protein match is unknown protein | ClassIIIA |
| | (TAIR: AT5G24890.1); Has 184 Blast hits to 184 proteins in 18 species: Archae - 0; Bacteria - | |
| | 0; Metazoa - 0; Fungi - 0; Plants - 184; Viruses - 0; Other Eukaryotes - 0 (source: NCBI | |
| | BLink). | |
| At1g02860 | BAH1, NLA, SPX (SYG1/Pho81/XPR1) domain-containing protein | ClassIIIA |
| At1g08830 | CSD1, copper/zinc superoxide dismutase 1 | ClassIIIA |
| At5g63800 | BGAL6, MUM2, Glycosyl hydrolase family 35 protein | ClassIIIA |
| At4g37790 | HAT22, Homeobox-leucine zipper protein family | ClassIIIA |
| At3g02150 | PTF1, TCP13, TFPD, plastid transcription factor 1 | ClassIIIA |
| At5g64460 | Phosphoglycerate mutase family protein | ClassIIIA |
| At2g33150 | KAT2, PED1, PKT3, peroxisomal 3-ketoacyl-CoA thiolase 3 | ClassIIIA |
| At1g06570 | HPD, PDS1, phytoene desaturation 1 | ClassIIIA |
| At3g14750 | unknown protein; BEST Arabidopsis thaliana protein match is unknown protein | ClassIIIA |
| | (TAIR: AT1G67170.1); Has 4036 Blast hits to 3091 proteins in 519 species: Archae - 61; | |
| | Bacteria - 669; Metazoa - 1503; Fungi - 255; Plants - 421; Viruses - 4; Other Eukaryotes - | |
| | 1123 (source: NCBI BLink). | |
| At1g18330 | EPR1, RVE7, Homeodomain-like superfamily protein | ClassIIIA |
| At3g49060 | U-box domain-containing protein kinase family protein | ClassIIIA |
| At3g16800 | Protein phosphatase 2C family protein | ClassIIIA |
| At1g72770 | HAB1, homology to ABI1 | ClassIIIA |
| At5g20050 | Protein kinase superfamily protein | ClassIIIA |
| At1g18260 | HCP-like superfamily protein | ClassIIIA |
| At2g26280 | CID7, CTC-interacting domain 7 | ClassIIIA |
| At5g13760 | Plasma-membrane choline transporter family protein | ClassIIIA |
| At1g55020 | ATLOX1, LOX1, lipoxygenase 1 | ClassIIIA |
| At5g03720 | AT-HSFA3, HSFA3, heat shock transcription factor A3 | ClassIIIA |
| At1g76240 | Arabidopsis protein of unknown function (DUF241) | ClassIIIA |
| At3g11340 | UDP-Glycosyltransferase superfamily protein | ClassIIIA |
| At3g16150 | N-terminal nucleophile aminohydrolases (Ntn hydrolases) superfamily protein | ClassIIIA |
| At2g34600 | JAZ7, TIFY5B, jasmonate-zim-domain protein 7 | ClassIIIA |
| At3g43430 | RING/U-box superfamily protein | ClassIIIA |
| At2g41200 | unknown protein; Has 26 Blast hits to 26 proteins in 11 species: Archae - 0; Bacteria - 0; | ClassIIIA |
| | Metazoa - 0; Fungi - 0; Plants - 26; Viruses - 0; Other Eukaryotes - 0 (source: NCBI BLink). | |
| At1g75230 | DNA glycosylase superfamily protein | ClassIIIA |
| At1g52240 | ATROPGEF11, PIRF1, ROPGEF11, RHO guanyl-nucleotide exchange factor 11 | ClassIIIA |
| At1g13080 | CYP71B2, cytochrome P450, family 71, subfamily B, polypeptide 2 | ClassIIIA |
| At1g68400 | leucine-rich repeat transmembrane protein kinase family protein | ClassIIIA |
| At1g56145 | Leucine-rich repeat transmembrane protein kinase | ClassIIIA |
| At5g61510 | GroES-like zinc-binding alcohol dehydrogenase family protein | ClassIIIA |
| At2g26600 | Glycosyl hydrolase superfamily protein | ClassIIIA |
| At1g02670 | P-loop containing nucleoside triphosphate hydrolases superfamily protein | ClassIIIA |
| At1g14340 | RNA-binding (RRM/RBD/RNP motifs) family protein | ClassIIIA |
| At2g41190 | Transmembrane amino acid transporter family protein | ClassIIIA |
| At1g06520 | ATGPAT1, GPAT1, glycerol-3-phosphate acyltransferase 1 | ClassIIIA |
| At1g23880 | NHL domain-containing protein | ClassIIIA |
| At3g52060 | Core-2/I-branching beta-1,6-N-acetylglucosaminyltransferase family protein | ClassIIIA |
| At1g08980 | AMI1, ATAMI1, ATTOC64-I, TOC64-I, amidase 1 | ClassIIIA |
| At5g37260 | CIR1, RVE2, Homeodomain-like superfamily protein | ClassIIIA |
| At4g23880 | unknown protein; Has 73 Blast hits to 69 proteins in 22 species: Archae - 0; Bacteria - 4; | ClassIIIA |
| | Metazoa - 9; Fungi - 2; Plants - 18; Viruses - 0; Other Eukaryotes - 40 (source: NCBI | |
| | BLink). | |
| At4g38200 | SEC7-like guanine nucleotide exchange family protein | ClassIIIA |
| At5g59590 | UGT76E2, UDP-glucosyl transferase 76E2 | ClassIIIA |
| At1g25275 | unknown protein; FUNCTIONS IN: molecular_function unknown; INVOLVED IN: | ClassIIIA |
| | response to karrikin; LOCATED IN: endomembrane system; EXPRESSED IN: 23 plant | |
| | structures; EXPRESSED DURING: 13 growth stages; Has 18 Blast hits to 18 proteins in 4 | |
| | species: Archae - 0; Bacteria - 0; Metazoa - 0; Fungi - 0; Plants - 18; Viruses - 0; Other | |
| | Eukaryotes - 0 (source: NCBI BLink). | |
| At2g29380 | HAI3, highly ABA-induced PP2C gene 3 | ClassIIIA |
| At1g08090 | ACH1, ATNRT2.1, ATNRT2:1, LIN1, NRT2, NRT2.1, NRT2:1, NRT2; 1AT, nitrate | ClassIIIA |
| | transporter 2:1 | |
| At5g57655 | xylose isomerase family protein | ClassIIIA |
| At4g01110 | unknown protein; BEST Arabidopsis thaliana protein match is unknown protein | ClassIIIA |
| | (TAIR: AT1G01453.1); Has 273 Blast hits to 272 proteins in 18 species: Archae - 0; Bacteria - | |
| | 0; Metazoa - 0; Fungi - 0; Plants - 273; Viruses - 0; Other Eukaryotes - 0 (source: NCBI | |
| | BLink). | |
| At3g54960 | ATPDI1, ATPDIL1-3, PDI1, PDIL1-3, PDI-like 1-3 | ClassIIIA |
| At3g54620 | ATBZIP25, BZIP25, BZO2H4, basic leucine zipper 25 | ClassIIIA |
| At1g03870 | FLA9, FASCICLIN-like arabinoogalactan 9 | ClassIIIA |
| At3g19400 | Cysteine proteinases superfamily protein | ClassIIIA |
| At3g13965 | pseudogene, hypothetical protein | ClassIIIA |
| At4g32960 | unknown protein; BEST Arabidopsis thaliana protein match is unknown protein | ClassIIIA |
| | (TAIR: AT4G32970.1); Has 106 Blast hits to 106 proteins in 39 species: Archae - 0; Bacteria - | |
| | 0; Metazoa - 62; Fungi - 0; Plants - 37; Viruses - 0; Other Eukaryotes - 7 (source: NCBI | |
| | BLink). | |
| At5g51850 | unknown protein; BEST Arabidopsis thaliana protein match is unknown protein | ClassIIIA |
| | (TAIR: AT5G62170.1); Has 384 Blast hits to 375 proteins in 79 species: Archae - 0; Bacteria - | |
| | 14; Metazoa - 135; Fungi - 31; Plants - 92; Viruses - 0; Other Eukaryotes - 112 (source: | |
| | NCBI BLink). | |
| At3g29240 | Protein of unknown function (DUF179) | ClassIIIA |
| At3g29160 | AKIN11, ATKIN11, KIN11, SNRK1.2, SNF1 kinase homolog 11 | ClassIIIA |
| At5g56100 | glycine-rich protein/oleosin | ClassIIIA |
| At5g47740 | Adenine nucleotide alpha hydrolases-like superfamily protein | ClassIIIA |
| At1g03100 | Pentatricopeptide repeat (PPR) superfamily protein | ClassIIIA |
| At1g67480 | Galactose oxidase/kelch repeat superfamily protein | ClassIIIA |
| At5g08350 | GRAM domain-containing protein/ABA-responsive protein-related | ClassIIIA |
| At3g23230 | Integrase-type DNA-binding superfamily protein | ClassIIIA |
| At4g28040 | nodulin MtN21/EamA-like transporter family protein | ClassIIIA |
| At5g47560 | ATSDAT, ATTDT, TDT, tonoplast dicarboxylate transporter | ClassIIIA |
| At5g04040 | SDP1, Patatin-like phospholipase family protein | ClassIIIA |
| At4g27480 | Core-2/I-branching beta-1,6-N-acetylglucosaminyltransferase family protein | ClassIIIA |
| At1g08930 | ERD6, Major facilitator superfamily protein | ClassIIIA |
| At3g15650 | alpha/beta-Hydrolases superfamily protein | ClassIIIA |
| At1g79700 | Integrase-type DNA-binding superfamily protein | ClassIIIA |
| At3g24520 | AT-HSFC1, HSFC1, heat shock transcription factor C1 | ClassIIIA |
| At4g36730 | GBF1, G-box binding factor 1 | ClassIIIA |
| At4g01030 | pentatricopeptide (PPR) repeat-containing protein | ClassIIIA |
| At1g79340 | AtMC4, MC4, metacaspase 4 | ClassIIIA |
| At1g10560 | ATPUB18, PUB18, plant U-box 18 | ClassIIIA |
| At2g43400 | ETFQO, electron-transfer flavoprotein:ubiquinone oxidoreductase | ClassIIIA |
| At5g56180 | ARP8, ARP8, ATARP8, actin-related protein 8 | ClassIIIA |
| At5g18170 | GDH1, glutamate dehydrogenase 1 | ClassIIIA |
| At4g16690 | ATMES16, MES16, methyl esterase 16 | ClassIIIA |
| At2g32510 | MAPKKK17, mitogen-activated protein kinase kinase kinase 17 | ClassIIIA |
| At1g76185 | unknown protein; BEST Arabidopsis thaliana protein match is unknown protein | ClassIIIA |
| | (TAIR: AT1G20460.1); Has 37 Blast hits to 37 proteins in 11 species: Archae - 0; Bacteria - | |
| | 0; Metazoa - 0; Fungi - 0; Plants - 37; Viruses - 0; Other Eukaryotes - 0 (source: NCBI | |
| | BLink). | |
| At2g44360 | unknown protein; Has 23 Blast hits to 23 proteins in 10 species: Archae - 0; Bacteria - 0; | ClassIIIA |
| | Metazoa - 0; Fungi - 0; Plants - 23; Viruses - 0; Other Eukaryotes - 0 (source: NCBI BLink). | |
| At3g45300 | ATIVD, IVD, IVDH, isovaleryl-CoA-dehydrogenase | ClassIIIA |
| At3g22920 | Cyclophilin-like peptidyl-prolyl cis-trans isomerase family protein | ClassIIIA |
| At4g39730 | Lipase/lipooxygenase, PLAT/LH2 family protein | ClassIIIA |
| At4g14500 | Polyketide cyclase/dehydrase and lipid transport superfamily protein | ClassIIIA |
| At3g14740 | RING/FYVE/PHD zinc finger superfamily protein | ClassIIIA |
| At3g13450 | DIN4, Transketolase family protein | ClassIIIA |
| At3g05200 | ATL6, RING/U-box superfamily protein | ClassIIIA |
| At2g28120 | Major facilitator superfamily protein | ClassIIIA |
| At2g02700 | Cysteine/Histidine-rich C1 domain family protein | ClassIIIA |
| At4g26290 | unknown protein; Has 9 Blast hits to 9 proteins in 5 species: Archae - 0; Bacteria - 0; | ClassIIIA |
| | Metazoa - 2; Fungi - 0; Plants - 3; Viruses - 0; Other Eukaryotes - 4 (source: NCBI BLink). | |
| At4g30170 | Peroxidase family protein | ClassIIIA |
| At3g11410 | AHG3, ATPP2CA, PP2CA, protein phosphatase 2CA | ClassIIIA |
| At1g10060 | ATBCAT-1, BCAT-1, branched-chain amino acid transaminase 1 | ClassIIIA |
| At1g63710 | CYP86A7, cytochrome P450, family 86, subfamily A, polypeptide 7 | ClassIIIA |
| At3g49940 | LBD38, LOB domain-containing protein 38 | ClassIIIA |
| At3g22930 | CML11, calmodulin-like 11 | ClassIIIA |
| At2g19320 | unknown protein; Has 9 Blast hits to 9 proteins in 4 species: Archae - 0; Bacteria - 0; | ClassIIIA |
| | Metazoa - 0; Fungi - 0; Plants - 9; Viruses - 0; Other Eukaryotes - 0 (source: NCBI BLink). | |
| At4g34350 | CLB6, HDR, ISPH, 4-hydroxy-3-methylbut-2-enyl diphosphate reductase | ClassIIIA |
| At5g61590 | Integrase-type DNA-binding superfamily protein | ClassIIIA |
| At2g28630 | KCS12, 3-ketoacyl-CoA synthase 12 | ClassIIIA |
| At2g19800 | MIOX2, myo-inositol oxygenase 2 | ClassIIIA |
| At3g56240 | CCH, copper chaperone | ClassIIIA |
| At1g56700 | Peptidase C15, pyroglutamyl peptidase I-like | ClassIIIA |
| At5g67440 | NPY3, Phototropic-responsive NPH3 family protein | ClassIIIA |
| At5g43190 | Galactose oxidase/kelch repeat superfamily protein | ClassIIIA |
| At2g15695 | Protein of unknown function DUF829, transmembrane 53 | ClassIIIA |
| At5g16110 | unknown protein; FUNCTIONS IN: molecular_function unknown; INVOLVED IN: | ClassIIIA |
| | biological_process unknown; LOCATED IN: chloroplast; EXPRESSED IN: 24 plant | |
| | structures; EXPRESSED DURING: 15 growth stages; BEST Arabidopsis thaliana protein | |
| | match is: unknown protein (TAIR: AT3G02555.1); Has 133 Blast hits to 133 proteins in 18 | |
| | species: Archae - 0; Bacteria - 0; Metazoa - 0; Fungi - 0; Plants - 133; Viruses - 0; Other | |
| | Eukaryotes - 0 (source: NCBI BLink). | |
| At1g66890 | FUNCTIONS IN: molecular_function unknown; INVOLVED IN: biological_process | ClassIIIA |
| | unknown; LOCATED IN: chloroplast; EXPRESSED IN: 22 plant structures; EXPRESSED | |
| | DURING: 13 growth stages; BEST Arabidopsis thaliana protein match is: 50S ribosomal | |
| | protein-related (TAIR: AT5G16200.1); Has 36 Blast hits to 36 proteins in 7 species: Archae - | |
| | 0; Bacteria - 0; Metazoa - 0; Fungi - 0; Plants - 36; Viruses - 0; Other Eukaryotes - 0 (source: | |
| | NCBI BLink). | |
| At3g57540 | Remorin family protein | ClassIIIA |
| At1g61740 | Sulfite exporter TauE/SafE family protein | ClassIIIA |
| At1g67470 | Protein kinase superfamily protein | ClassIIIA |
| At5g49440 | unknown protein; Has 30201 Blast hits to 17322 proteins in 780 species: Archae - 12; | ClassIIIA |
| | Bacteria - 1396; Metazoa - 17338; Fungi - 3422; Plants - 5037; Viruses - 0; Other Eukaryotes - | |
| | 2996 (source: NCBI BLink). | |
| At4g01870 | tolB protein-related | ClassIIIA |
| At4g21440 | ATM4, ATMYB102, MYB102, MYB102, MYB-like 102 | ClassIIIA |
| At4g29950 | Ypt/Rab-GAP domain of gyp1p superfamily protein | ClassIIIA |
| At3g51860 | ATCAX3, ATHCX1, CAX1-LIKE, CAX3, cation exchanger 3 | ClassIIIA |
| At1g16150 | WAKL4, wall associated kinase-like 4 | ClassIIIA |
| At1g67880 | beta-1,4-N-acetylglucosaminyltransferase family protein | ClassIIIA |
| At1g08630 | THA1, threonine aldolase 1 | ClassIIIA |
| At1g28130 | GH3.17, Auxin-responsive GH3 family protein | ClassIIIA |
| At3g55150 | ATEXO70H1, EXO70H1, exocyst subunit exo70 family protein H1 | ClassIIIA |
| At1g76160 | sks5, SKU5 similar 5 | ClassIIIA |
| At4g37220 | Cold acclimation protein WCOR413 family | ClassIIIA |
| At2g31380 | STH, salt tolerance homologue | ClassIIIA |
| At3g14050 | AT-RSH2, ATRSH2, RSH2, RELA/SPOT homolog 2 | ClassIIIA |
| At3g14770 | Nodulin MtN3 family protein | ClassIIIA |
| At5g57630 | CIPK21, SnRK3.4, CBL-interacting protein kinase 21 | ClassIIIA |
| At5g24530 | DMR6, 2-oxoglutarate (2OG) and Fe(II)-dependent oxygenase superfamily protein | ClassIIIA |
| At3g56000 | ATCSLA14, CSLA14, cellulose synthase like A14 | ClassIIIA |
| At1g15060 | Uncharacterised conserved protein UCP031088, alpha/beta hydrolase | ClassIIIA |
| At2g28200 | C2H2-type zinc finger family protein | ClassIIIA |
| At4g33420 | Peroxidase superfamily protein | ClassIIIA |
| At5g18650 | CHY-type/CTCHY-type/RING-type Zinc finger protein | ClassIIIA |
| At1g66070 | Translation initiation factor eIF3 subunit | ClassIIIA |
| At2g10640 | transposable element gene | ClassIIIA |
| At5g18610 | Protein kinase superfamily protein | ClassIIIA |
| At4g15620 | Uncharacterised protein family (UPF0497) | ClassIIIA |
| At5g50200 | ATNRT3.1, NRT3.1, WR3, nitrate transmembrane transporters | ClassIIIA |
| At4g01330 | Protein kinase superfamily protein | ClassIIIA |
| At5g46590 | anac096, NAC096, NAC domain containing protein 96 | ClassIIIA |
| At2g39570 | ACT domain-containing protein | ClassIIIA |
| At5g04740 | ACT domain-containing protein | ClassIIIA |
| At1g08920 | ESL1, ERD (early response to dehydration) six-like 1 | ClassIIIA |
| At1g09460 | Carbohydrate-binding X8 domain superfamily protein | ClassIIIA |
| At4g38060 | unknown protein; BEST Arabidopsis thaliana protein match is unknown protein | ClassIIIA |
| | (TAIR: AT5G65480.1); Has 63 Blast hits to 63 proteins in 13 species: Archae - 0; Bacteria - | |
| | 0; Metazoa - 0; Fungi - 0; Plants - 63; Viruses - 0; Other Eukaryotes - 0 (source: NCBI | |
| | BLink). | |
| At3g57420 | Protein of unknown function (DUF288) | ClassIIIA |
| At5g54080 | HGO, homogentisate 1,2-dioxygenase | ClassIIIA |
| At3g06780 | glycine-rich protein | ClassIIIA |
| At2g22080 | unknown protein; Has 96314 Blast hits to 34847 proteins in 1702 species: Archae - 612; | ClassIIIA |
| | Bacteria - 27969; Metazoa - 24311; Fungi - 12153; Plants - 4409; Viruses - 1572; Other | |
| | Eukaryotes - 25288 (source: NCBI BLink). | |
| At1g49670 | NQR, ARP protein (REF) | ClassIIIA |
| At2g03740 | late embryogenesis abundant domain-containing protein/LEA domain-containing protein | ClassIIIA |
| At5g56870 | BGAL4, beta-galactosidase 4 | ClassIIIA |
| At4g33150 | LKR, LKR/SDH, SDH, lysine-ketoglutarate reductase/saccharopine dehydrogenase | ClassIIIA |
| | bifunctional enzyme | |
| At1g23550 | SRO2, similar to RCD one 2 | ClassIIIA |
| At2g12400 | unknown protein; FUNCTIONS IN: molecular_function unknown; INVOLVED IN: | ClassIIIA |
| | biological_process unknown; LOCATED IN: endomembrane system; EXPRESSED IN: 25 | |
| | plant structures; EXPRESSED DURING: 13 growth stages; BEST Arabidopsis thaliana | |
| | protein match is: unknown protein (TAIR: AT2G25270.1); Has 177 Blast hits to 172 proteins | |
| | in 23 species: Archae - 0; Bacteria - 2; Metazoa - 3; Fungi - 0; Plants - 164; Viruses - 0; | |
| | Other Eukaryotes - 8 (source: NCBI BLink). | |
| At1g12080 | Vacuolar calcium-binding protein-related | ClassIIIA |
| At5g01590 | unknown protein; FUNCTIONS IN: molecular_function unknown; INVOLVED IN: | ClassIIIA |
| | biological_process unknown; LOCATED IN: chloroplast, chloroplast envelope; | |
| | EXPRESSED IN: 22 plant structures; EXPRESSED DURING: 13 growth stages; Has 60 | |
| | Blast hits to 59 proteins in 31 species: Archae - 0; Bacteria - 20; Metazoa - 1; Fungi - 2; | |
| | Plants - 33; Viruses - 0; Other Eukaryotes - 4 (source: NCBI BLink). | |
| At4g19810 | Glycosyl hydrolase family protein with chitinase insertion domain | ClassIIIA |
| At3g17440 | ATNPSN13, NPSN13, novel plant snare 13 | ClassIIIA |
| At5g03350 | Legume lectin family protein | ClassIIIA |
| At2g44670 | Protein of unknown function (DUF581) | ClassIIIA |
| At5g28050 | Cytidine/deoxycytidylate deaminase family protein | ClassIIIA |
| At5g10450 | 14-3-3lambda, AFT1, GRF6, G-box regulating factor 6 | ClassIIIA |
| At4g23870 | unknown protein; BEST Arabidopsis thaliana protein match is unknown protein | ClassIIIA |
| | (TAIR: AT4G11020.1); Has 12 Blast hits to 12 proteins in 4 species: Archae - 0; Bacteria - 0; | |
| | Metazoa - 0; Fungi - 0; Plants - 12; Viruses - 0; Other Eukaryotes - 0 (source: NCBI BLink). | |
| At1g69910 | Protein kinase superfamily protein | ClassIIIA |
| At5g13110 | G6PD2, glucose-6-phosphate dehydrogenase 2 | ClassIIIA |
| At1g14330 | Galactose oxidase/kelch repeat superfamily protein | ClassIIIA |
| At1g06560 | NOL1/NOP2/sun family protein | ClassIIIA |
| At3g16170 | AMP-dependent synthetase and ligase family protein | ClassIIIA |
| At5g20250 | DIN10, Raffinose synthase family protein | ClassIIIA |
| At5g49690 | UDP-Glycosyltransferase superfamily protein | ClassIIIA |
| At1g07250 | UGT71C4, UDP-glucosyl transferase 71C4 | ClassIIIA |
| At3g51540 | unknown protein; BEST Arabidopsis thaliana protein match is unknown protein | ClassIIIA |
| | (TAIR: AT3G08670.1); Has 22744 Blast hits to 9965 proteins in 783 species: Archae - 64; | |
| | Bacteria - 2760; Metazoa - 8515; Fungi - 3864; Plants - 499; Viruses - 702; Other Eukaryotes - | |
| | 6340 (source: NCBI BLink). | |
| At3g30396 | transposable element gene | ClassIIIA |
| At1g67510 | Leucine-rich repeat protein kinase family protein | ClassIIIA |
| At2g39130 | Transmembrane amino acid transporter family protein | ClassIIIA |
| At5g23050 | AAE17, acyl-activating enzyme 17 | ClassIIIA |
| At1g22360 | AtUGT85A2, UGT85A2, UDP-glucosyl transferase 85A2 | ClassIIIA |
| At2g32660 | AtRLP22, RLP22, receptor like protein 22 | ClassIIIA |
| At1g54740 | Protein of unknown function (DUF3049) | ClassIIIA |
| At1g03080 | kinase interacting (KIP1-like) family protein | ClassIIIA |
| At4g38490 | unknown protein; Has 30201 Blast hits to 17322 proteins in 780 species: Archae - 12; | ClassIIIA |
| | Bacteria - 1396; Metazoa - 17338; Fungi - 3422; Plants - 5037; Viruses - 0; Other Eukaryotes - | |
| | 2996 (source: NCBI BLink). | |
| At4g36790 | Major facilitator superfamily protein | ClassIIIA |
| At4g38480 | Transducin/WD40 repeat-like superfamily protein | ClassIIIA |
| At3g61070 | PEX11E, peroxin 11E | ClassIIIA |
| At3g45060 | ATNRT2.6, NRT2.6, high affinity nitrate transporter 2.6 | ClassIIIA |
| At4g33910 | 2-oxoglutarate (2OG) and Fe(II)-dependent oxygenase superfamily protein | ClassIIIA |
| At1g58180 | ATBCA6, BCA6, beta carbonic anhydrase 6 | ClassIIIA |
| At1g71980 | Protease-associated (PA) RING/U-box zinc finger family protein | ClassIIIA |
| At1g57680 | FUNCTIONS IN: molecular_function unknown; INVOLVED IN: biological_process | ClassIIIA |
| | unknown; LOCATED IN: endomembrane system; EXPRESSED IN: 24 plant structures; | |
| | EXPRESSED DURING: 15 growth stages; CONTAINS InterPro DOMAIN/s: | |
| | Uncharacterised conserved protein UCP031277 (InterPro: IPR016971); Has 70 Blast hits to | |
| | 70 proteins in 19 species: Archae - 0; Bacteria - 0; Metazoa - 1; Fungi - 0; Plants - 66; | |
| | Viruses - 0; Other Eukaryotes - 3 (source: NCBI BLink). | |
| At3g46280 | protein kinase-related | ClassIIIA |
| At1g30820 | CTP synthase family protein | ClassIIIA |
| At3g13460 | ECT2, evolutionarily conserved C-terminal region 2 | ClassIIIA |
| At4g17140 | pleckstrin homology (PH) domain-containing protein | ClassIIIA |
| At5g16120 | alpha/beta-Hydrolases superfamily protein | ClassIIIA |
| At1g04410 | Lactate/malate dehydrogenase family protein | ClassIIIA |
| At4g27260 | GH3.5, WES1, Auxin-responsive GH3 family protein | ClassIIIA |
| At1g66470 | RHD6, ROOT HAIR DEFECTIVE6 | ClassIIIA |
| At2g02040 | ATPTR2, ATPTR2-B, NTR1, PTR2, PTR2-B, peptide transporter 2 | ClassIIIA |
| At3g05390 | FUNCTIONS IN: molecular_function unknown; INVOLVED IN: biological_process | ClassIIIA |
| | unknown; LOCATED IN: mitochondrion; EXPRESSED IN: 15 plant structures; | |
| | EXPRESSED DURING: 7 growth stages; CONTAINS InterPro DOMAIN/s: Protein of | |
| | unknown function DUF248, methyltransferase putative (InterPro: IPR004159); BEST | |
| | Arabidopsis thaliana protein match is: S-adenosyl-L-methionine-dependent | |
| | methyltransferases superfamily protein (TAIR: AT4G01240.1); Has 507 Blast hits to 498 | |
| | proteins in 33 species: Archae - 4; Bacteria - 8; Metazoa - 0; Fungi - 0; Plants - 493; Viruses - | |
| | 0; Other Eukaryotes - 2 (source: NCBI BLink). | |
| At4g03510 | ATRMA1, RMA1, RING membrane-anchor 1 | ClassIIIA |
| At3g20860 | ATNEK5, NEK5, NIMA-related kinase 5 | ClassIIIA |
| At3g62650 | unknown protein; BEST Arabidopsis thaliana protein match is: unknown protein | ClassIIIA |
| | (TAIR: AT2G47485.1); Has 57 Blast hits to 57 proteins in 13 species: Archae - 0; Bacteria - | |
| | 0; Metazoa - 0; Fungi - 0; Plants - 57; Viruses - 0; Other Eukaryotes - 0 (source: NCBI | |
| | BLink). | |
| At1g54100 | ALDH7B4, aldehyde dehydrogenase 7B4 | ClassIIIA |
| At3g47500 | CDF3, cycling DOF factor 3 | ClassIIIA |
| At5g13750 | ZIFL1, zinc induced facilitator-like 1 | ClassIIIA |
| At3g51730 | saposin B domain-containing protein | ClassIIIA |
| At1g67810 | SUFE2, sulfur E2 | ClassIIIA |
| At3g52490 | Double Clp-N motif-containing P-loop nucleoside triphosphate hydrolases superfamily | ClassIIIA |
| | protein | |
| At3g48690 | ATCXE12, CXE12, alpha/beta-Hydrolases superfamily protein | ClassIIIA |
| At3g55450 | PBL1, PBS1-like 1 | ClassIIIA |
| At1g68620 | alpha/beta-Hydrolases superfamily protein | ClassIIIA |
| At3g54140 | ATPTR1, PTR1, peptide transporter 1 | ClassIIIA |
| At4g24330 | Protein of unknown function (DUF1682) | ClassIIIA |
| At1g64010 | Serine protease inhibitor (SERPIN) family protein | ClassIIIA |
| At2g46270 | GBF3, G-box binding factor 3 | ClassIIIA |
| At5g10210 | CONTAINS InterPro DOMAIN/s: C2 calcium-dependent membrane targeting | ClassIIIA |
| | (InterPro: IPR000008); BEST Arabidopsis thaliana protein match is: unknown protein | |
| | (TAIR: AT5G65030.1); Has 1807 Blast hits to 1807 proteins in 277 species: Archae - 0; | |
| | Bacteria - 0; Metazoa - 736; Fungi - 347; Plants - 385; Viruses - 0; Other Eukaryotes - 339 | |
| | (source: NCBI BLink). | |
| At1g73260 | ATKTI1, KTI1, kunitz trypsin inhibitor 1 | ClassIIIA |
| At1g75800 | Pathogenesis-related thaumatin superfamily protein | ClassIIIA |
| At5g07080 | HXXXD-type acyl-transferase family protein | ClassIIIA |
| At1g21310 | ATEXT3, EXT3, RSH, extensin 3 | ClassIIIA |
| At1g61810 | BGLU45, beta-glucosidase 45 | ClassIIIA |
| At4g32300 | SD2-5, S-domain-2 5 | ClassIIIA |
| At1g65840 | ATPAO4, PAO4, polyamine oxidase 4 | ClassIIIA |
| At5g47390 | myb-like transcription factor family protein | ClassIIIA |
| At5g61600 | ERF104, ethylene response factor 104 | ClassIIIA |
| At5g24030 | SLAH3, SLAC1 homologue 3 | ClassIIIA |
| At5g15190 | unknown protein; FUNCTIONS IN: molecular_function unknown; INVOLVED IN: | ClassIIIA |
| | biological_process unknown; LOCATED IN: chloroplast; EXPRESSED IN: 17 plant | |
| | structures; EXPRESSED DURING: LP.04 four leaves visible, 4 anthesis, petal | |
| | differentiation and expansion stage, E expanded cotyledon stage, D bilateral stage; Has 7 | |
| | Blast hits to 7 proteins in 3 species: Archae - 0; Bacteria - 0; Metazoa - 0; Fungi - 0; Plants - | |
| | 7; Viruses - 0; Other Eukaryotes - 0 (source: NCBI BLink). | |
| At4g38340 | NLP3; Plant regulator RWP-RK family protein | ClassIIIA |
| At1g10070 | ATBCAT-2, BCAT-2, branched-chain amino acid transaminase 2 | ClassIIIA |
| At2g19350 | Eukaryotic protein of unknown function (DUF872) | ClassIIIA |
| At4g31240 | protein kinase C-like zinc finger protein | ClassIIIA |
| At5g40450 | unknown protein; FUNCTIONS IN: molecular_function unknown; INVOLVED IN: | ClassIIIA |
| | biological_process unknown; LOCATED IN: chloroplast, plasma membrane; EXPRESSED | |
| | IN: 24 plant structures; EXPRESSED DURING: 13 growth stages; Has 30201 Blast hits to | |
| | 17322 proteins in 780 species: Archae - 12; Bacteria - 1396; Metazoa - 17338; Fungi - 3422; | |
| | Plants - 5037; Viruses - 0; Other Eukaryotes - 2996 (source: NCBI BLink). | |
| At1g69570 | Dof-type zinc finger DNA-binding family protein | ClassIIIA |
| At1g11260 | ATSTP1, STP1, sugar transporter 1 | ClassIIIA |
| At4g37540 | LBD39, LOB domain-containing protein 39 | ClassIIIA |
| At3g20410 | CPK9, calmodulin-domain protein kinase 9 | ClassIIIA |
| At5g27920 | F-box family protein | ClassIIIA |
| At4g01026 | PYL7, RCAR2, PYR1-like 7 | ClassIIIA |
| At4g35780 | ACT-like protein tyrosine kinase family protein | ClassIIIA |
| At3g06850 | BCE2, DIN3, LTA1, 2-oxoacid dehydrogenases acyltransferase family protein | ClassIIIA |
| At1g76410 | ATL8, RING/U-box superfamily protein | ClassIIIA |
| At1g20340 | DRT112, PETE2, Cupredoxin superfamily protein | ClassIIIA |
| At1g55510 | BCDH BETA1, branched-chain alpha-keto acid decarboxylase E1 beta subunit | ClassIIIA |
| At4g35770 | ATSEN1, DIN1, SEN1, SEN1, Rhodanese/Cell cycle control phosphatase superfamily | ClassIIIA |
| | protein | |
| At5g47240 | atnudt8, NUDT8, nudix hydrolase homolog 8 | ClassIIIA |
| At3g14760 | unknown protein; FUNCTIONS IN: molecular_function unknown; INVOLVED IN: | ClassIIIA |
| | biological_process unknown; LOCATED IN: endomembrane system; EXPRESSED IN: 6 | |
| | plant structures; EXPRESSED DURING: LP.04 four leaves visible, LP.02 two leaves visible; | |
| | Has 63 Blast hits to 63 proteins in 13 species: Archae - 0; Bacteria - 0; Metazoa - 0; Fungi - | |
| | 0; Plants - 63; Viruses - 0; Other Eukaryotes - 0 (source: NCBI BLink). | |
| At3g60690 | SAUR-like auxin-responsive protein family | ClassIIIA |
| At1g32460 | unknown protein; Has 19 Blast hits to 19 proteins in 8 species: Archae - 0; Bacteria - 0; | ClassIIIA |
| | Metazoa - 0; Fungi - 0; Plants - 19; Viruses - 0; Other Eukaryotes - 0 (source: NCBI BLink). | |
| At2g35230 | IKU1, IKU1, VQ motif-containing protein | ClassIIIA |
| At5g54500 | FQR1, flavodoxin-like quinone reductase 1 | ClassIIIA |
| At5g43830 | Aluminium induced protein with YGL and LRDR motifs | ClassIIIA |
| At1g51820 | Leucine-rich repeat protein kinase family protein | ClassIIIA |
| At1g63180 | UGE3, UDP-D-glucose/UDP-D-galactose 4-epimerase 3 | ClassIIIA |
| At3g61260 | Remorin family protein | ClassIIIA |
| At2g38750 | ANNAT4, annexin 4 | ClassIIIA |
| At4g32870 | Polyketide cyclase/dehydrase and lipid transport superfamily protein | ClassIIIA |
| At3g47960 | Major facilitator superfamily protein | ClassIIIA |
| At5g05340 | Peroxidase superfamily protein | ClassIIIA |
| At2g38400 | AGT3, alanine:glyoxylate aminotransferase 3 | ClassIIIA |
| At5g66030 | ATGRIP, GRIP, Golgi-localized GRIP domain-containing protein | ClassIIIA |
| At3g56360 | unknown protein; FUNCTIONS IN: molecular_function unknown; INVOLVED IN: | ClassIIIA |
| | biological_process unknown; LOCATED IN: chloroplast; EXPRESSED IN: 24 plant | |
| | structures; EXPRESSED DURING: 15 growth stages; BEST Arabidopsis thaliana protein | |
| | match is: unknown protein (TAIR: AT5G05250.1); Has 45 Blast hits to 45 proteins in 13 | |
| | species: Archae - 0; Bacteria - 0; Metazoa - 0; Fungi - 0; Plants - 45; Viruses - 0; Other | |
| | Eukaryotes - 0 (source: NCBI BLink). | |
| At5g18850 | unknown protein; FUNCTIONS IN: molecular_function unknown; INVOLVED IN: | ClassIIIA |
| | biological_process unknown; LOCATED IN: endomembrane system; EXPRESSED IN: 23 | |
| | plant structures; EXPRESSED DURING: 13 growth stages; Has 1807 Blast hits to 1807 | |
| | proteins in 277 species: Archae - 0; Bacteria - 0; Metazoa - 736; Fungi - 347; Plants - 385; | |
| | Viruses - 0; Other Eukaryotes - 339 (source: NCBI BLink). | |
| At2g31390 | pfkB-like carbohydrate kinase family protein | ClassIIIA |
| At5g03550 | BEST Arabidopsis thaliana protein match is: TRAF-like family protein | ClassIIIA |
| | (TAIR: AT2G42460.1); Has 137 Blast hits to 125 proteins in 2 species: Archae - 0; Bacteria - | |
| | 0; Metazoa - 0; Fungi - 0; Plants - 137; Viruses - 0; Other Eukaryotes - 0 (source: NCBI | |
| | BLink). | |
| At1g42480 | unknown protein; FUNCTIONS IN: molecular_function unknown; INVOLVED IN: | ClassIIIA |
| | biological_process unknown; LOCATED IN: endomembrane system; EXPRESSED IN: 24 | |
| | plant structures; EXPRESSED DURING: 13 growth stages; CONTAINS InterPro | |
| | DOMAIN/s: Protein of unknown function DUF3456 (InterPro: IPR021852); Has 177 Blast | |
| | hits to 177 proteins in 59 species: Archae - 0; Bacteria - 0; Metazoa - 140; Fungi - 0; Plants - | |
| | 35; Viruses - 0; Other Eukaryotes - 2 (source: NCBI BLink). | |
| At4g30490 | AFG1-like ATPase family protein | ClassIIIA |
| At2g25900 | ATCTH, ATTZF1, Zinc finger C-x8-C-x5-C-x3-H type family protein | ClassIIIA |
| At3g54630 | CONTAINS InterPro DOMAIN/s: Kinetochore protein Ndc80 (InterPro: IPR005550); Has | ClassIIIA |
| | 24780 Blast hits to 15608 proteins in 1321 species: Archae - 545; Bacteria - 2969; Metazoa - | |
| | 12597; Fungi - 2181; Plants - 1581; Viruses - 39; Other Eukaryotes - 4868 (source: NCBI | |
| | BLink). | |
| At1g66550 | ATWRKY67, WRKY67, WRKY DNA-binding protein 67 | ClassIIIA |
| At4g39780 | Integrase-type DNA-binding superfamily protein | ClassIIIA |
| At1g75450 | ATCKX5, ATCKX6, CKX5, cytokinin oxidase 5 | ClassIIIA |
| At2g01570 | RGA, RGA1, GRAS family transcription factor family protein | ClassIIIA |
| At4g38470 | ACT-like protein tyrosine kinase family protein | ClassIIIA |
| At1g35580 | CINV1, cytosolic invertase 1 | ClassIIIA |
| At1g11380 | PLAC8 family protein | ClassIIIA |
| At1g48840 | Plant protein of unknown function (DUF639) | ClassIIIA |
| At1g60940 | SNRK2-10, SNRK2.10, SRK2B, SNF1-related protein kinase 2.10 | ClassIIIA |
| At1g31480 | SGR2, shoot gravitropism 2 (SGR2) | ClassIIIA |
| At3g19390 | Granulin repeat cysteine protease family protein | ClassIIIA |
| At4g15545 | unknown protein; BEST Arabidopsis thaliana protein match is: unknown protein | ClassIIIA |
| | (TAIR: AT1G16520.1); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At1g32200 | ACT1, ATS1, phospholipid/glycerol acyltransferase family protein | ClassIIIA |
| At1g61660 | basic helix-loop-helix (bHLH) DNA-binding superfamily protein | ClassIIIA |
| At1g18270 | ketose-bisphosphate aldolase class-II family protein | ClassIIIA |
| At5g59220 | HAI1, highly ABA-induced PP2C gene 1 | ClassIIIA |
| At5g48430 | Eukaryotic aspartyl protease family protein | ClassIIIA |
| At5g06690 | WCRKC1, WCRKC thioredoxin 1 | ClassIIIA |
| At2g40170 | ATEM6, EM6, GEA6, Stress induced protein | ClassIIIA |
| At5g06980 | unknown protein; BEST Arabidopsis thaliana protein match is: unknown protein | ClassIIIA |
| | (TAIR: AT3G12320.1); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At3g03470 | CYP89A9, cytochrome P450, family 87, subfamily A, polypeptide 9 | ClassIIIA |
| At1g67070 | DIN9, PMI2, Mannose-6-phosphate isomerase, type I | ClassIIIA |
| At5g05440 | PYL5, RCAR8, Polyketide cyclase/dehydrase and lipid transport superfamily protein | ClassIIIA |
| At1g80460 | GLI1, NHO1, Actin-like ATPase superfamily protein | ClassIIIA |
| At2g39210 | Major facilitator superfamily protein | ClassIIIA |
| At5g63620 | GroES-like zinc-binding alcohol dehydrogenase family protein | ClassIIIA |
| At1g73240 | CONTAINS InterPro DOMAIN/s: Nucleoporin protein Ndc1-Nup (InterPro: IPR019049); | ClassIIIA |
| | Has 36 Blast hits to 36 proteins in 17 species: Archae - 0; Bacteria - 0; Metazoa - 1; Fungi - | |
| | 0; Plants - 35; Viruses - 0; Other Eukaryotes - 0 (source: NCBI BLink). | |
| At2g30600 | BTB/POZ domain-containing protein | ClassIIIA |
| At5g04310 | Pectin lyase-like superfamily protein | ClassIIIA |
| At4g18340 | Glycosyl hydrolase superfamily protein | ClassIIIA |
| At5g16960 | Zinc-binding dehydrogenase family protein | ClassIIIA |
| At4g15630 | Uncharacterised protein family (UPF0497) | ClassIIIA |
| At2g03220 | ATFT1, ATFUT1, FT1, MUR2, fucosyltransferase 1 | ClassIIIA |
| At3g50780 | BEST Arabidopsis thaliana protein match is: BTB/POZ domain-containing protein | ClassIIIA |
| | (TAIR: AT1G63850.1); Has 298 Blast hits to 298 proteins in 22 species: Archae - 0; Bacteria - | |
| | 0; Metazoa - 10; Fungi - 0; Plants - 287; Viruses - 0; Other Eukaryotes - 1 (source: NCBI | |
| | BLink). | |
| At5g65630 | GTE7, global transcription factor group E7 | ClassIIIA |
| At1g28260 | Telomerase activating protein Est1 | ClassIIIA |
| At3g02550 | LBD41, LOB domain-containing protein 41 | ClassIIIA |
| At3g14067 | Subtilase family protein | ClassIIIA |
| At5g26740 | Protein of unknown function (DUF300) | ClassIIIA |
| At4g36670 | Major facilitator superfamily protein | ClassIIIA |
| At1g19700 | BEL10, BLH10, BEL1-like homeodomain 10 | ClassIIIA |
| At5g64260 | EXL2, EXORDIUM like 2 | ClassIIIA |
| At1g75220 | Major facilitator superfamily protein | ClassIIIA |
| At2g40420 | Transmembrane amino acid transporter family protein | ClassIIIA |
| At1g30900 | BP80-3; 3, VSR3; 3, VSR6, VACUOLAR SORTING RECEPTOR 6 | ClassIIIA |
| At5g20885 | RING/U-box superfamily protein | ClassIIIA |
| At5g52250 | Transducin/WD40 repeat-like superfamily protein | ClassIIIA |
| At3g46440 | UXS5, UDP-XYL synthase 5 | ClassIIIA |
| At5g13740 | ZIF1, zinc induced facilitator 1 | ClassIIIA |
| At1g11780 | oxidoreductase, 2OG-Fe (II) oxygenase family protein | ClassIIIA |
| At5g43430 | ETFBETA, electron transfer flavoprotein beta | ClassIIIA |
| At5g60200 | TMO6, TARGET OF MONOPTEROS 6 | ClassIIIA |
| At5g16970 | AER, AT-AER, alkenal reductase | ClassIIIA |
| At3g57020 | Calcium-dependent phosphotriesterase superfamily protein | ClassIIIA |
| At5g02780 | GSTL1, glutathione transferase lambda 1 | ClassIIIA |
| At5g39040 | ALS1, ATTAP2, TAP2, transporter associated with antigen processing protein 2 | ClassIIIA |
| At5g19090 | Heavy metal transport/detoxification superfamily protein | ClassIIIA |
| At4g24220 | AWI31, VEP1, NAD(P)-binding Rossmann-fold superfamily protein | ClassIIIA |
| At1g03790 | SOM, Zinc finger C-x8-C-x5-C-x3-H type family protein | ClassIIIA |
| At2g38820 | Protein of unknown function (DUF506) | ClassIIIA |
| At1g20300 | Pentatricopeptide repeat (PPR) superfamily protein | ClassIIIA |
| At3g46690 | UDP-Glycosyltransferase superfamily protein | ClassIIIA |
| At3g15610 | Transducin/WD40 repeat-like superfamily protein | ClassIIIA |
| At3g01175 | Protein of unknown function (DUF1666) | ClassIIIA |
| At1g76990 | ACR3, ACT domain repeat 3 | ClassIIIA |
| At1g68410 | Protein phosphatase 2C family protein | ClassIIIA |
| At5g27350 | SFP1, Major facilitator superfamily protein | ClassIIIA |
| At4g32320 | APX6, ascorbate peroxidase 6 | ClassIIIA |
| At5g11520 | ASP3, YLS4, aspartate aminotransferase 3 | ClassIIIA |
| At2g14170 | ALDH6B2, aldehyde dehydrogenase 6B2 | ClassIIIA |
| At1g63700 | EMB71, MAPKKK4, YDA, Protein kinase superfamily protein | ClassIIIA |
| At1g68850 | Peroxidase superfamily protein | ClassIIIA |
| At3g15260 | Protein phosphatase 2C family protein | ClassIIIA |
| At5g04630 | CYP77A9, cytochrome P450, family 77, subfamily A, polypeptide 9 | ClassIIIA |
| At3g01270 | Pectate lyase family protein | ClassIIIA |
| At1g26730 | EXS (ERD1/XPR1/SYG1) family protein | ClassIIIA |
| At2g37440 | DNAse I-like superfamily protein | ClassIIIA |
| At5g49650 | XK-2, XK2, xylulose kinase-2 | ClassIIIA |
| At1g26270 | Phosphatidylinositol 3- and 4-kinase family protein | ClassIIIA |
| At5g28610 | BEST Arabidopsis thaliana protein match is: glycine-rich protein (TAIR: AT5G28630.1); Has | ClassIIIA |
| | 1536 Blast hits to 1202 proteins in 136 species: Archae - 0; Bacteria - 8; Metazoa - 888; | |
| | Fungi - 120; Plants - 71; Viruses - 39; Other Eukaryotes - 410 (source: NCBI BLink). | |
| At5g04770 | ATCAT6, CAT6, cationic amino acid transporter 6 | ClassIIIA |
| At4g10840 | Tetratricopeptide repeat (TPR)-like superfamily protein | ClassIIIA |
| At2g43060 | IBH1, ILI1 binding bHLH 1 | ClassIIIA |
| At4g03080 | BSL1, BRI1 suppressor 1 (BSU1)-like 1 | ClassIIIA |
| At5g57660 | ATCOL5, COL5, CONSTANS-like 5 | ClassIIIA |
| At5g07070 | CIPK2, SnRK3.2, CBL-interacting protein kinase 2 | ClassIIIA |
| At4g15550 | IAGLU, indole-3-acetate beta-D-glucosyltransferase | ClassIIIA |
| At2g01860 | EMB975, Tetratricopeptide repeat (TPR)-like superfamily protein | ClassIIIA |
| At5g58620 | zinc finger (CCCH-type) family protein | ClassIIIA |
| At1g15050 | IAA34, indole-3-acetic acid inducible 34 | ClassIIIA |
| At5g66400 | ATDI8, RAB18, Dehydrin family protein | ClassIIIA |
| At2g19810 | CCCH-type zinc finger family protein | ClassIIIA |
| At3g17420 | GPK1, glyoxysomal protein kinase 1 | ClassIIIA |
| At3g47640 | PYE, basic helix-loop-helix (bHLH) DNA-binding superfamily protein | ClassIIIA |
| At3g53150 | UGT73D1, UDP-glucosyl transferase 73D1 | ClassIIIA |
| At5g67320 | HOS15, WD-40 repeat family protein | ClassIIIA |
| At3g17110 | pseudogene, glycine-rich protein | ClassIIIA |
| At3g61060 | AtPP2-A13, PP2-A13, phloem protein 2-A13 | ClassIIIA |
| At1g01490 | Heavy metal transport/detoxification superfamily protein | ClassIIIA |
| At5g41610 | ATCHX18, CHX18, cation/H+ exchanger 18 | ClassIIIA |
| At3g57890 | Tubulin binding cofactor C domain-containing protein | ClassIIIA |
| At4g17950 | AT hook motif DNA-binding family protein | ClassIIIA |
| At4g01120 | ATBZIP54, GBF2, G-box binding factor 2 | ClassIIIA |
| At3g51840 | ACX4, ATG6, ATSCX, acyl-CoA oxidase 4 | ClassIIIA |
| At4g32950 | Protein phosphatase 2C family protein | ClassIIIA |
| At4g24060 | Dof-type zinc finger DNA-binding family protein | ClassIIIA |
| At1g79350 | EMB1135, RING/FYVE/PHD zinc finger superfamily protein | ClassIIIA |
| At2g39980 | HXXXD-type acyl-transferase family protein | ClassIIIA |
| At3g15950 | NAI2, DNA topoisomerase-related | ClassIIIA |
| At2g27490 | ATCOAE, dephospho-CoA kinase family | ClassIIIA |
| At3g60510 | ATP-dependent caseinolytic (Clp) protease/crotonase family protein | ClassIIIA |
| At3g28510 | P-loop containing nucleoside triphosphate hydrolases superfamily protein | ClassIIIA |
| At4g39070 | B-box zinc finger family protein | ClassIIIA |
| At1g22400 | ATUGT85A1, UGT85A1, UDP-Glycosyltransferase superfamily protein | ClassIIIA |
| At2g02800 | APK2B, protein kinase 2B | ClassIIIA |
| At4g14420 | HR-like lesion-inducing protein-related | ClassIIIA |
| At4g30550 | Class I glutamine amidotransferase-like superfamily protein | ClassIIIA |
| At1g03610 | Protein of unknown function (DUF789) | ClassIIIA |
| At2g23450 | Protein kinase superfamily protein | ClassIIIA |
| At4g13430 | ATLEUC1, IIL1, isopropyl malate isomerase large subunit 1 | ClassIIIA |
| At3g19920 | unknown protein; BEST Arabidopsis thaliana protein match is: unknown protein | ClassIIIA |
| | (TAIR: AT5G64230.1); Has 217 Blast hits to 217 proteins in 16 species: Archae - 0; Bacteria - | |
| | 2; Metazoa - 0; Fungi - 0; Plants - 215; Viruses - 0; Other Eukaryotes - 0 (source: NCBI | |
| | BLink). | |
| At5g49360 | ATBXL1, BXL1, beta-xylosidase 1 | ClassIIIA |
| At1g29760 | Putative adipose-regulatory protein (Seipin) | ClassIIIA |
| At4g38500 | Protein of unknown function (DUF616) | ClassIIIA |
| At1g15380 | Lactoylglutathione lyase/glyoxalase I family protein | ClassIIIA |
| At2g17500 | Auxin efflux carrier family protein | ClassIIIA |
| At5g24470 | APRR5, PRR5, pseudo-response regulator 5 | ClassIIIA |
| At1g03090 | MCCA, methylcrotonyl-CoA carboxylase alpha chain, mitochondrial/3-methylcrotonyl- | ClassIIIA |
| | CoA carboxylase 1 (MCCA) | |
| At3g18980 | ETP1, EIN2 targeting protein1 | ClassIIIA |
| At3g16910 | AAE7, ACN1, acyl-activating enzyme 7 | ClassIIIA |
| At1g17190 | ATGSTU26, GSTU26, glutathione S-transferase tau 26 | ClassIIIA |
| At5g18630 | alpha/beta-Hydrolases superfamily protein | ClassIIIA |
| At5g17640 | Protein of unknown function (DUF1005) | ClassIIIA |

ClassIIIB. NR: no binding but repression

| | | |
|---|---|---|
| At1g56510 | ADR2, WRR4, Disease resistance protein (TIR-NBS-LRR class) | ClassIIIB |
| At1g74710 | ATICS1, EDS16, ICS1, SID2, ADC synthase superfamily protein | ClassIIIB |
| At2g17040 | anac036, NAC036, NAC domain containing protein 36 | ClassIIIB |
| At1g57630 | Toll-Interleukin-Resistance (TIR) domain family protein | ClassIIIB |
| At3g63390 | unknown protein; Has 30201 Blast hits to 17322 proteins in 780 species: Archae - 12; | ClassIIIB |
| | Bacteria - 1396; Metazoa - 17338; Fungi - 3422; Plants - 5037; Viruses - 0; Other Eukaryotes - | |
| | 2996 (source: NCBI BLink). | |
| At1g67050 | unknown protein; BEST Arabidopsis thaliana protein match is: unknown protein | ClassIIIB |
| | (TAIR: AT5G38320.1); Has 617 Blast hits to 318 proteins in 80 species: Archae - 0; Bacteria - | |
| | 16; Metazoa - 141; Fungi - 62; Plants - 128; Viruses - 2; Other Eukaryotes - 268 (source: | |
| | NCBI BLink). | |
| At1g73750 | Uncharacterised conserved protein UCP031088, alpha/beta hydrolase | ClassIIIB |
| At3g05490 | RALFL22, ralf-like 22 | ClassIIIB |
| At1g15890 | Disease resistance protein (CC-NBS-LRR class) family | ClassIIIB |
| At2g46590 | DAG2, Dof-type zinc finger DNA-binding family protein | ClassIIIB |
| At2g44450 | BGLU15, beta glucosidase 15 | ClassIIIB |
| At1g05800 | DGL, alpha/beta-Hydrolases superfamily protein | ClassIIIB |
| At1g32690 | unknown protein; FUNCTIONS IN: molecular_function unknown; INVOLVED IN: | ClassIIIB |
| | biological_process unknown; LOCATED IN: plasma membrane; EXPRESSED IN: 21 plant | |
| | structures; EXPRESSED DURING: 11 growth stages; BEST Arabidopsis thaliana protein | |
| | match is: unknown protein (TAIR: AT2G35200.1); Has 45 Blast hits to 45 proteins in 8 | |
| | species: Archae - 0; Bacteria - 0; Metazoa - 0; Fungi - 0; Plants - 45; Viruses - 0; Other | |
| | Eukaryotes - 0 (source: NCBI BLink). | |
| At5g44350 | ethylene-responsive nuclear protein-related | ClassIIIB |
| At4g30560 | ATCNGC9, CNGC9, cyclic nucleotide gated channel 9 | ClassIIIB |
| At4g26120 | Ankyrin repeat family protein/BTB/POZ domain-containing protein | ClassIIIB |
| At3g10630 | UDP-Glycosyltransferase superfamily protein | ClassIIIB |
| At4g39890 | AtRABH1c, RABH1c, RAB GTPase homolog H1C | ClassIIIB |
| At3g61390 | RING/U-box superfamily protein | ClassIIIB |
| At3g07390 | AIR12, auxin-responsive family protein | ClassIIIB |
| At2g23270 | unknown protein; FUNCTIONS IN: molecular_function unknown; INVOLVED IN: | ClassIIIB |
| | biological_process unknown; LOCATED IN: endomembrane system; EXPRESSED IN: | |
| | stem, sperm cell, root, stamen; EXPRESSED DURING: 4 anthesis; BEST Arabidopsis | |
| | thaliana protein match is: unknown protein (TAIR: AT4G37290.1); Has 36 Blast hits to 35 | |
| | proteins in 6 species: Archae - 0; Bacteria - 0; Metazoa - 0; Fungi - 0; Plants - 36; Viruses - | |
| | 0; Other Eukaryotes - 0 (source: NCBI BLink). | |
| At4g22820 | A20/AN1-like zinc finger family protein | ClassIIIB |
| At1g51620 | Protein kinase superfamily protein | ClassIIIB |
| At4g39940 | AKN2, APK2, APS-kinase 2 | ClassIIIB |
| At1g10160 | transposable element gene | ClassIIIB |
| At3g19630 | Radical SAM superfamily protein | ClassIIIB |
| At2g44090 | Ankyrin repeat family protein | ClassIIIB |
| At1g58080 | ATATP-PRT1, ATP-PRT1, HISN1A, ATP phosphoribosyl transferase 1 | ClassIIIB |
| At3g55960 | Haloacid dehalogenase-like hydrolase (HAD) superfamily protein | ClassIIIB |
| At3g48850 | PHT3; 2, phosphate transporter 3; 2 | ClassIIIB |
| At1g53980 | Ubiquitin-like superfamily protein | ClassIIIB |
| At1g74430 | ATMYB95, ATMYBCP66, MYB95, myb domain protein 95 | ClassIIIB |
| At5g40540 | Protein kinase superfamily protein | ClassIIIB |
| At4g14368 | Regulator of chromosome condensation (RCC1) family protein | ClassIIIB |
| At2g16500 | ADC1, ARGDC, ARGDC1, SPE1, arginine decarboxylase 1 | ClassIIIB |
| At3g05360 | AtRLP30, RLP30, receptor like protein 30 | ClassIIIB |
| At1g20510 | OPCL1, OPC-8:0 CoA ligase1 | ClassIIIB |
| At3g17020 | Adenine nucleotide alpha hydrolases-like superfamily protein | ClassIIIB |
| At2g42360 | RING/U-box superfamily protein | ClassIIIB |
| At1g24625 | ZFP7, zinc finger protein 7 | ClassIIIB |
| At5g41550 | Disease resistance protein (TIR-NBS-LRR class) family | ClassIIIB |
| At2g41380 | S-adenosyl-L-methionine-dependent methyltransferases superfamily protein | ClassIIIB |
| At5g65870 | ATPSK5, PSK5, PSK5, phytosulfokine 5 precursor | ClassIIIB |
| At4g11850 | MEE54, PLDGAMMA1, phospholipase D gamma 1 | ClassIIIB |
| At3g13650 | Disease resistance-responsive (dirigent-like protein) family protein | ClassIIIB |
| At5g56760 | ATSERAT1; 1, SAT-52, SAT5, SERAT1; 1, serine acetyltransferase 1; 1 | ClassIIIB |
| At1g75540 | STH2, salt tolerance homolog2 | ClassIIIB |
| At1g53430 | Leucine-rich repeat transmembrane protein kinase | ClassIIIB |
| At1g74590 | ATGSTU10, GSTU10, glutathione 5-transferase TAU 10 | ClassIIIB |
| At5g52670 | Copper transport protein family | ClassIIIB |
| At3g44735 | ATPSK3, PSK1, PSK3, PHYTOSULFOKINE 3 PRECURSOR | ClassIIIB |
| At3g18250 | Putative membrane lipoprotein | ClassIIIB |
| At1g28190 | unknown protein; BEST Arabidopsis thaliana protein match is: unknown protein | ClassIIIB |
| | (TAIR: AT5G12340.1); Has 166 Blast hits to 162 proteins in 36 species: Archae - 0; Bacteria - | |
| | 2; Metazoa - 15; Fungi - 5; Plants - 124; Viruses - 0; Other Eukaryotes - 20 (source: NCBI | |
| | BLink). | |
| At3g02770 | Ribonuclease E inhibitor RraA/Dimethylmenaquinone methyltransferase | ClassIIIB |
| At5g25190 | Integrase-type DNA-binding superfamily protein | ClassIIIB |
| At4g00330 | CRCK2, calmodulin-binding receptor-like cytoplasmic kinase 2 | ClassIIIB |
| At1g53050 | Protein kinase superfamily protein | ClassIIIB |
| At1g05060 | unknown protein; Has 34 Blast hits to 34 proteins in 13 species: Archae - 0; Bacteria - 0; | ClassIIIB |
| | Metazoa - 0; Fungi - 0; Plants - 34; Viruses - 0; Other Eukaryotes - 0 (source: NCBI BLink). | |
| At3g09020 | alpha 1,4-glycosyltransferase family protein | ClassIIIB |
| At1g30040 | ATGA2OX2, GA2OX2, GA2OX2, gibberellin 2-oxidase | ClassIIIB |
| At5g24430 | Calcium-dependent protein kinase (CDPK) family protein | ClassIIIB |
| At4g21390 | B120, S-locus lectin protein kinase family protein | ClassIIIB |
| At1g70130 | Concanavalin A-like lectin protein kinase family protein | ClassIIIB |
| At2g04160 | AIR3, Subtilisin-like serine endopeptidase family protein | ClassIIIB |
| At3g20510 | Transmembrane proteins 14C | ClassIIIB |
| At3g10640 | VPS60.1, SNF7 family protein | ClassIIIB |
| At5g58787 | RING/U-box superfamily protein | ClassIIIB |
| At2g34920 | EDA18, RING/U-box superfamily protein | ClassIIIB |
| At1g44130 | Eukaryotic aspartyl protease family protein | ClassIIIB |
| At4g37940 | AGL21, AGAMOUS-like 21 | ClassIIIB |
| At4g27720 | Major facilitator superfamily protein | ClassIIIB |
| At5g22530 | unknown protein; BEST Arabidopsis thaliana protein match is: unknown protein | ClassIIIB |
| | (TAIR: AT5G22520.1); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At1g17310 | MADS-box transcription factor family protein | ClassIIIB |
| At1g35560 | TCP family transcription factor | ClassIIIB |
| At4g40020 | Myosin heavy chain-related protein | ClassIIIB |
| At1g24140 | Matrixin family protein | ClassIIIB |
| At1g11210 | Protein of unknown function (DUF761) | ClassIIIB |
| At1g48320 | Thioesterase superfamily protein | ClassIIIB |
| At5g12880 | proline-rich family protein | ClassIIIB |
| At1g10650 | SBP (S-ribonuclease binding protein) family protein | ClassIIIB |
| At3g09270 | ATGSTU8, GSTU8, glutathione S-transferase TAU 8 | ClassIIIB |
| At1g29250 | Alba DNA/RNA-binding protein | ClassIIIB |
| At3g61850 | DAG1, Dof-type zinc finger DNA-binding family protein | ClassIIIB |
| At1g78100 | F-box family protein | ClassIIIB |
| At4g00080 | UNE11, Plant invertase/pectin methylesterase inhibitor superfamily protein | ClassIIIB |
| At1g32350 | AOX1D, alternative oxidase 1D | ClassIIIB |
| At3g49350 | Ypt/Rab-GAP domain of gyp1p superfamily protein | ClassIIIB |
| At1g80530 | Major facilitator superfamily protein | ClassIIIB |
| At1g55610 | BRL1, BRI1 like | ClassIIIB |
| At5g13870 | EXGT-A4, XTH5, xyloglucan endotransglucosylase/hydrolase 5 | ClassIIIB |
| At4g28085 | unknown protein; Has 30201 Blast hits to 17322 proteins in 780 species: Archae - 12; | ClassIIIB |
| | Bacteria - 1396; Metazoa - 17338; Fungi - 3422; Plants - 5037; Viruses - 0; Other Eukaryotes - | |
| | 2996 (source: NCBI BLink). | |
| At1g07750 | RmlC-like cupins superfamily protein | ClassIIIB |
| At3g50480 | HR4, homolog of RPW8 4 | ClassIIIB |
| At3g21230 | 4CL5, 4-coumarate:CoA ligase 5 | ClassIIIB |
| At5g60350 | unknown protein; Has 110 Blast hits to 97 proteins in 36 species: Archae - 0; Bacteria - 10; | ClassIIIB |
| | Metazoa - 39; Fungi - 2; Plants - 5; Viruses - 0; Other Eukaryotes - 54 (source: NCBI | |
| | BLink). | |
| At3g09000 | proline-rich family protein | ClassIIIB |
| At3g25070 | RIN4, RPM1 interacting protein 4 | ClassIIIB |
| At3g11840 | PUB24, plant U-box 24 | ClassIIIB |
| At2g11520 | CRCK3, calmodulin-binding receptor-like cytoplasmic kinase 3 | ClassIIIB |
| At5g24540 | BGLU31, beta glucosidase 31 | ClassIIIB |
| At2g19130 | S-locus lectin protein kinase family protein | ClassIIIB |
| At5g48540 | receptor-like protein kinase-related family protein | ClassIIIB |
| At4g24160 | alpha/beta-Hydrolases superfamily protein | ClassIIIB |
| At1g09940 | HEMA2, Glutamyl-tRNA reductase family protein | ClassIIIB |
| At3g59080 | Eukaryotic aspartyl protease family protein | ClassIIIB |
| At3g27110 | Peptidase family M48 family protein | ClassIIIB |
| At4g16780 | ATHB-2, ATHB2, HAT4, HB-2, homeobox protein 2 | ClassIIIB |
| At5g44070 | ARA8, ATPCS1, CAD1, PCS1, phytochelatin synthase 1 (PCS1) | ClassIIIB |
| At5g66880 | SNRK2-3, SNRK2.3, SRK2I, sucrose nonfermenting 1(SNF1)-related protein kinase 2.3 | ClassIIIB |
| At5g49620 | AtMYB78, MYB78, myb domain protein 78 | ClassIIIB |
| At5g22550 | Plant protein of unknown function (DUF247) | ClassIIIB |
| At3g21080 | ABC transporter-related | ClassIIIB |
| At3g03020 | unknown protein; FUNCTIONS IN: molecular_function unknown; INVOLVED IN: | ClassIIIB |
| | biological_process unknown; LOCATED IN: chloroplast; EXPRESSED IN: 21 plant | |
| | structures; EXPRESSED DURING: 13 growth stages; Has 5 Blast hits to 5 proteins in 1 | |
| | species: Archae - 0; Bacteria - 0; Metazoa - 0; Fungi - 0; Plants - 5; Viruses - 0; Other | |
| | Eukaryotes - 0 (source: NCBI BLink). | |
| At5g59510 | DVL18, RTFL5, ROTUNDIFOLIA like 5 | ClassIIIB |
| At3g53730 | Histone superfamily protein | ClassIIIB |
| At1g19220 | ARF11, ARF19, IAA22, auxin response factor 19 | ClassIIIB |
| At1g18890 | ATCDPK1, CDPK1, CPK10, calcium-dependent protein kinase 1 | ClassIIIB |
| At3g44720 | ADT4, arogenate dehydratase 4 | ClassIIIB |
| At4g11170 | Disease resistance protein (TIR-NBS-LRR class) family | ClassIIIB |
| At5g07620 | Protein kinase superfamily protein | ClassIIIB |
| At3g54980 | Pentatricopeptide repeat (PPR) superfamily protein | ClassIIIB |
| At5g06720 | ATPA2, PA2, peroxidase 2 | ClassIIIB |
| At5g41100 | FUNCTIONS IN: molecular_function unknown; INVOLVED IN: biological_process | ClassIIIB |
| | unknown; LOCATED IN: plasma membrane; EXPRESSED IN: 23 plant structures; | |
| | EXPRESSED DURING: 13 growth stages; BEST Arabidopsis thaliana protein match is: | |
| | hydroxyproline-rich glycoprotein family protein (TAIR: AT3G26910.2); Has 1503 Blast hits | |
| | to 1197 proteins in 220 species: Archae - 4; Bacteria - 108; Metazoa - 481; Fungi - 318; | |
| | Plants - 186; Viruses - 39; Other Eukaryotes - 367 (source: NCBI BLink). | |
| At4g02360 | Protein of unknown function, DUF538 | ClassIIIB |
| At4g09570 | ATCPK4, CPK4, calcium-dependent protein kinase 4 | ClassIIIB |
| At1g51940 | protein kinase family protein/peptidoglycan-binding LysM domain-containing protein | ClassIIIB |
| At5g65020 | ANNAT2, annexin 2 | ClassIIIB |
| At3g48090 | ATEDS1, EDS1, alpha/beta-Hydrolases superfamily protein | ClassIIIB |
| At1g70530 | CRK3, cysteine-rich RLK (RECEPTOR-like protein kinase) 3 | ClassIIIB |
| At4g12070 | unknown protein; INVOLVED IN: biological_process unknown; LOCATED IN: plasma | ClassIIIB |
| | membrane; EXPRESSED IN: 22 plant structures; EXPRESSED DURING: 13 growth stages; | |
| | Has 30201 Blast hits to 17322 proteins in 780 species: Archae - 12; Bacteria - 1396; Metazoa - | |
| | 17338; Fungi - 3422; Plants - 5037; Viruses - 0; Other Eukaryotes - 2996 (source: NCBI | |
| | BLink). | |
| At1g63040 | a pseudogene member of the DREB subfamily A-4 of ERF/AP2 transcription factor family. | ClassIIIB |
| | The translated product contains one AP2 domain. There are 17 members in this subfamily | |
| | including TINY. | |
| At2g01150 | RHA2B, RING-H2 finger protein 2B | ClassIIIB |
| At4g25030 | unknown protein; BEST Arabidopsis thaliana protein match is: unknown protein | ClassIIIB |
| | (TAIR: AT5G45410.3); Has 125 Blast hits to 125 proteins in 36 species: Archae - 2; Bacteria - | |
| | 31; Metazoa - 0; Fungi - 4; Plants - 88; Viruses - 0; Other Eukaryotes - 0 (source: NCBI | |
| | BLink). | |
| At2g32030 | Acyl-CoA N-acyltransferases (NAT) superfamily protein | ClassIIIB |
| At3g60910 | S-adenosyl-L-methionine-dependent methyltransferases superfamily protein | ClassIIIB |
| At1g68150 | ATWRKY9, WRKY9, WRKY DNA-binding protein 9 | ClassIIIB |
| At2g06050 | DDE1, OPR3, oxophytodienoate-reductase 3 | ClassIIIB |
| At5g62680 | Major facilitator superfamily protein | ClassIIIB |
| At5g45750 | AtRABA1c, RABA1c, RAB GTPase homolog A1C | ClassIIIB |
| At4g18890 | BEH3, BES1/BZR1 homolog 3 | ClassIIIB |
| At2g27390 | proline-rich family protein | ClassIIIB |
| At4g23440 | Disease resistance protein (TIR-NBS class) | ClassIIIB |
| At2g22680 | Zinc finger (C3HC4-type RING finger) family protein | ClassIIIB |
| At3g54040 | PAR1 protein | ClassIIIB |
| At4g37730 | AtbZIP7, bZIP7, basic leucine-zipper 7 | ClassIIIB |
| At4g30080 | ARF16, auxin response factor 16 | ClassIIIB |
| At3g43250 | Family of unknown function (DUF572) | ClassIIIB |
| At2g46150 | Late embryogenesis abundant (LEA) hydroxyproline-rich glycoprotein family | ClassIIIB |
| At5g61210 | ATSNAP33, ATSNAP33B, SNAP33, SNP33, soluble N-ethylmaleimide-sensitive factor | ClassIIIB |
| | adaptor protein 33 | |
| At5g57340 | unknown protein; Has 30201 Blast hits to 17322 proteins in 780 species: Archae - 12; | ClassIIIB |
| | Bacteria - 1396; Metazoa - 17338; Fungi - 3422; Plants - 5037; Viruses - 0; Other Eukaryotes - | |
| | 2996 (source: NCBI BLink). | |
| At5g07870 | HXXXD-type acyl-transferase family protein | ClassIIIB |
| At5g54170 | Polyketide cyclase/dehydrase and lipid transport superfamily protein | ClassIIIB |
| At1g13340 | Regulator of Vps4 activity in the MVB pathway protein | ClassIIIB |
| At5g48175 | FUNCTIONS IN: molecular_function unknown; INVOLVED IN: biological_process | ClassIIIB |
| | unknown; LOCATED IN: endomembrane system; EXPRESSED IN: hypocotyl, male | |
| | gametophyte, root; BEST Arabidopsis thaliana protein match is: Glycosyl hydrolase | |
| | superfamily protein (TAIR: AT3G09260.1); Has 30201 Blast hits to 17322 proteins in 780 | |
| | species: Archae - 12; Bacteria - 1396; Metazoa - 17338; Fungi - 3422; Plants - 5037; Viruses - | |
| | 0; Other Eukaryotes - 2996 (source: NCBI BLink). | |
| At1g07130 | ATSTN1, STN1, Nucleic acid-binding, OB-fold-like protein | ClassIIIB |
| At2g30130 | ASL5, LBD12, PCK1, Lateral organ boundaries (LOB) domain family protein | ClassIIIB |
| At4g17230 | SCL13, SCARECROW-like 13 | ClassIIIB |
| At3g05510 | Phospholipid/glycerol acyltransferase family protein | ClassIIIB |
| At1g18570 | AtMYB51, BW51A, BW51B, HIG1, MYB51, myb domain protein 51 | ClassIIIB |
| At3g27160 | GHS1, Ribosomal protein S21 family protein | ClassIIIB |
| At2g39700 | ATEXP4, ATEXPA4, ATHEXP ALPHA 1.6, EXPA4, expansin A4 | ClassIIIB |
| At4g40080 | ENTH/ANTH/VHS superfamily protein | ClassIIIB |
| At1g57560 | AtMYB50, MYB50, myb domain protein 50 | ClassIIIB |
| At2g25250 | unknown protein; FUNCTIONS IN: molecular_function unknown; INVOLVED IN: | ClassIIIB |
| | biological_process unknown; LOCATED IN: chloroplast; EXPRESSED IN: 23 plant | |
| | structures; EXPRESSED DURING: 14 growth stages; BEST Arabidopsis thaliana protein | |
| | match is: unknown protein (TAIR: AT4G32020.1); Has 30 Blast hits to 30 proteins in 7 | |
| | species: Archae - 0; Bacteria - 0; Metazoa - 0; Fungi - 2; Plants - 28; Viruses - 0; Other | |
| | Eukaryotes - 0 (source: NCBI BLink). | |
| At2g28570 | unknown protein; Has 13 Blast hits to 13 proteins in 6 species: Archae - 0; Bacteria - 0; | ClassIIIB |
| | Metazoa - 0; Fungi - 0; Plants - 13; Viruses - 0; Other Eukaryotes - 0 (source: NCBI BLink). | |
| At1g66090 | Disease resistance protein (TIR-NBS class) | ClassIIIB |
| At1g44100 | AAPS, amino acid permease 5 | ClassIIIB |
| At3g11820 | AT-SYR1, ATSYP121, ATSYR1, PEN1, SYP121, SYR1, syntaxin of plants 121 | ClassIIIB |
| At4g01850 | AtSAM2, MAT2, SAM-2, SAM2, S-adenosylmethionine synthetase 2 | ClassIIIB |
| At2g24240 | BTB/POZ domain with WD40/YVTN repeat-like protein | ClassIIIB |
| At1g32310 | unknown protein; Has 28 Blast hits to 28 proteins in 9 species: Archae - 0; Bacteria - 0; | ClassIIIB |
| | Metazoa - 0; Fungi - 0; Plants - 28; Viruses - 0; Other Eukaryotes - 0 (source: NCBI BLink). | |
| At5g67570 | DG1, EMB1408, EMB246, Tetratricopeptide repeat (TPR)-like superfamily protein | ClassIIIB |
| At4g11370 | RHA1A, RING-H2 finger A1A | ClassIIIB |
| At1g60030 | ATNAT7, NAT7, nucleobase-ascorbate transporter 7 | ClassIIIB |
| At1g18860 | ATWRKY61, WRKY61, WRKY DNA-binding protein 61 | ClassIIIB |
| At1g18580 | GAUT11, galacturonosyltransferase 11 | ClassIIIB |
| At1g79160 | unknown protein; BEST Arabidopsis thaliana protein match is unknown protein | ClassIIIB |
| | (TAIR: AT1G16500.1); Has 104 Blast hits to 102 proteins in 13 species: Archae - 0; Bacteria - | |
| | 0; Metazoa - 0; Fungi - 0; Plants - 104; Viruses - 0; Other Eukaryotes - 0 (source: NCBI | |
| | BLink). | |
| At2g19710 | Regulator of Vps4 activity in the MVB pathway protein | ClassIIIB |
| At4g01720 | AtWRKY47, WRKY47, WRKY family transcription factor | ClassIIIB |
| At2g37840 | Protein kinase superfamily protein | ClassIIIB |
| At4g39840 | unknown protein; FUNCTIONS IN: molecular_function unknown; INVOLVED IN: | ClassIIIB |
| | biological_process unknown; LOCATED IN: endomembrane system; EXPRESSED IN: 23 | |
| | plant structures; EXPRESSED DURING: 13 growth stages; Has 20719 Blast hits to 6096 | |
| | proteins in 607 species: Archae - 22; Bacteria - 3243; Metazoa - 4364; Fungi - 2270; Plants - | |
| | 237; Viruses - 128; Other Eukaryotes - 10455 (source: NCBI BLink). | |
| At4g32060 | calcium-binding EF hand family protein | ClassIIIB |
| At1g70940 | ATPIN3, PIN3, Auxin efflux carrier family protein | ClassIIIB |
| At2g26290 | ARSK1, root-specific kinase 1 | ClassIIIB |
| At1g44830 | Integrase-type DNA-binding superfamily protein | ClassIIIB |
| At5g43520 | Cysteine/Histidine-rich C1 domain family protein | ClassIIIB |
| At4g28350 | Concanavalin A-like lectin protein kinase family protein | ClassIIIB |
| At2g20960 | pEARLI4, Arabidopsis phospholipase-like protein (PEARLI 4) family | ClassIIIB |
| At3g49220 | Plant invertase/pectin methylesterase inhibitor superfamily | ClassIIIB |
| At5g52240 | AtMAPR5, ATMP1, MSBP1, membrane steroid binding protein 1 | ClassIIIB |
| At1g09520 | LOCATED IN: chloroplast; EXPRESSED IN: 21 plant structures; EXPRESSED DURING: | ClassIIIB |
| | 12 growth stages; CONTAINS InterPro DOMAIN/s: Zinc finger, PHD-type, conserved site | |
| | (InterPro: IPR019786); BEST Arabidopsis thaliana protein match is: PHD finger family | |
| | protein (TAIR: AT3G17460.1); Has 56 Blast hits to 56 proteins in 17 species: Archae - 0; | |
| | Bacteria - 2; Metazoa - 0; Fungi - 4; Plants - 46; Viruses - 0; Other Eukaryotes - 4 (source: | |
| | NCBI BLink). | |
| At1g04440 | CKL13, casein kinase like 13 | ClassIIIB |
| At3g08750 | F-box and associated interaction domains-containing protein | ClassIIIB |
| At4g17260 | Lactate/malate dehydrogenase family protein | ClassIIIB |
| At3g63410 | APG1, E37, IEP37, VTE3, S-adenosyl-L-methionine-dependent methyltransferases ClassIIIB | |
| | superfamily protein | |
| At3g23820 | GAE6, UDP-D-glucuronate 4-epimerase 6 | ClassIIIB |
| At1g51920 | unknown protein; FUNCTIONS IN: molecular_function unknown; INVOLVED IN: | ClassIIIB |
| | biological_process unknown; LOCATED IN: endomembrane system; EXPRESSED IN: | |
| | stem, stamen; EXPRESSED DURING: 4 anthesis; Has 22 Blast hits to 22 proteins in 5 | |
| | species: Archae - 0; Bacteria - 0; Metazoa - 0; Fungi - 0; Plants - 22; Viruses - 0; Other | |
| | Eukaryotes - 0 (source: NCBI BLink). | |
| At4g34180 | Cyclase family protein | ClassIIIB |
| At1g52560 | HSP20-like chaperones superfamily protein | ClassIIIB |
| At3g49720 | unknown protein; FUNCTIONS IN: molecular_function unknown; INVOLVED IN: | ClassIIIB |
| | biological_process unknown; LOCATED IN: chloroplast thylakoid membrane, Golgi | |
| | apparatus, plasma membrane, membrane; EXPRESSED IN: 25 plant structures; | |
| | EXPRESSED DURING: 15 growth stages; BEST Arabidopsis thaliana protein match is: | |
| | unknown protein (TAIR: AT5G65810.1); Has 64 Blast hits to 64 proteins in 11 species: | |
| | Archae - 0; Bacteria - 0; Metazoa - 0; Fungi - 0; Plants - 64; Viruses - 0; Other Eukaryotes - 0 | |
| | (source: NCBI BLink). | |
| At3g28740 | CYP81D1, Cytochrome P450 superfamily protein | ClassIIIB |
| At3g52360 | unknown protein; FUNCTIONS IN: molecular_function unknown; INVOLVED IN: | ClassIIIB |
| | response to karrikin; LOCATED IN: endomembrane system; EXPRESSED IN: 23 plant | |
| | structures; EXPRESSED DURING: 14 growth stages; BEST Arabidopsis thaliana protein | |
| | match is: unknown protein (TAIR: AT2G35850.1); Has 34 Blast hits to 34 proteins in 10 | |
| | species: Archae - 0; Bacteria - 0; Metazoa - 0; Fungi - 0; Plants - 34; Viruses - 0; Other | |
| | Eukaryotes - 0 (source: NCBI BLink). | |
| At3g17700 | ATCNGC20, CNBT1, CNGC20, cyclic nucleotide-binding transporter 1 | ClassIIIB |
| At4g33300 | ADR1-L1, ADR1-like 1 | ClassIIIB |
| At3g52400 | ATSYP122, SYP122, syntaxin of plants 122 | ClassIIIB |
| At3g20900 | unknown protein; Has 2 Blast hits to 2 proteins in 1 species: Archae - 0; Bacteria - 0; | ClassIIIB |
| | Metazoa - 0; Fungi - 0; Plants - 2; Viruses - 0; Other Eukaryotes - 0 (source: NCBI BLink). | |
| At5g14930 | SAG101, senescence-associated gene 101 | ClassIIIB |
| At1g35200 | 60S ribosomal protein L4/L1 (RPL4B), pseudogene, similar to 60S ribosomal protein L4 | ClassIIIB |
| | (fragment) GB: P49691 from (Arabidopsis thaliana); blastp match of 50% identity and 6.3e−17 | |
| | P-value to SP\|Q9XF97\|RL4_PRUAR 60S ribosomal protein L4 (L1). (Apricot) {Prunus | |
| | armeniaca} | |
| At5g38310 | unknown protein; Has 1807 Blast hits to 1807 proteins in 277 species: Archae - 0; Bacteria - | ClassIIIB |
| | 0; Metazoa - 736; Fungi - 347; Plants - 385; Viruses - 0; Other Eukaryotes - 339 (source: | |
| | NCBI BLink). | |
| At3g23090 | TPX2 (targeting protein for Xklp2) protein family | ClassIIIB |
| At5g63770 | ATDGK2, DGK2, diacylglycerol kinase 2 | ClassIIIB |
| At5g13190 | CONTAINS InterPro DOMAIN/s: LPS-induced tumor necrosis factor alpha factor | ClassIIIB |
| | (InterPro: IPR006629); Has 30201 Blast hits to 17322 proteins in 780 species: Archae - 12; | |
| | Bacteria - 1396; Metazoa - 17338; Fungi - 3422; Plants - 5037; Viruses - 0; Other Eukaryotes - | |
| | 2996 (source: NCBI BLink). | |
| At4g30470 | NAD(P)-binding Rossmann-fold superfamily protein | ClassIIIB |
| At1g29860 | ATWRKY71, WRKY71, WRKY DNA-binding protein 71 | ClassIIIB |
| At4g28940 | Phosphorylase superfamily protein | ClassIIIB |
| At1g72070 | Chaperone DnaJ-domain superfamily protein | ClassIIIB |
| At2g45080 | cycp3; 1, cyclin p3; 1 | ClassIIIB |
| At2g01880 | ATPAP7, PAP7, purple acid phosphatase 7 | ClassIIIB |
| At1g34750 | Protein phosphatase 2C family protein | ClassIIIB |
| At1g09920 | TRAF-type zinc finger-related | ClassIIIB |
| At2g38010 | Neutral/alkaline non-lysosomal ceramidase | ClassIIIB |
| At1g21830 | unknown protein; CONTAINS InterPro DOMAIN/s: Protein of unknown function DUF740 | ClassIIIB |
| | (InterPro: IPR008004); BEST Arabidopsis thaliana protein match is: unknown protein | |
| | (TAIR: AT1G44608.1); Has 49 Blast hits to 49 proteins in 12 species: Archae - 0; Bacteria - | |
| | 0; Metazoa - 0; Fungi - 0; Plants - 49; Viruses - 0; Other Eukaryotes - 0 (source: NCBI | |
| | BLink). | |
| At1g74870 | RING/U-box superfamily protein | ClassIIIB |
| At3g10190 | Calcium-binding EF-hand family protein | ClassIIIB |
| At4g37400 | CYP81F3, cytochrome P450, family 81, subfamily F, polypeptide 3 | ClassIIIB |
| At1g07000 | ATEXO70B2, EXO70B2, exocyst subunit exo70 family protein B2 | ClassIIIB |
| At1g73066 | Leucine-rich repeat family protein | ClassIIIB |
| At2g39530 | Uncharacterised protein family (UPF0497) | ClassIIIB |
| At5g62070 | IQD23, IQ-domain 23 | ClassIIIB |
| At3g45640 | ATMAPK3, ATMPK3, MPK3, mitogen-activated protein kinase 3 | ClassIIIB |
| At1g11000 | ATMLO4, MLO4, Seven transmembrane MLO family protein | ClassIIIB |
| At2g26480 | UGT76D1, UDP-glucosyl transferase 76D1 | ClassIIIB |
| At4g02200 | Drought-responsive family protein | ClassIIIB |
| At5g07310 | Integrase-type DNA-binding superfamily protein | ClassIIIB |
| At2g16430 | ATPAP10, PAP10, purple acid phosphatase 10 | ClassIIIB |
| At5g44610 | MAP18, PCAP2, microtubule-associated protein 18 | ClassIIIB |
| At4g36680 | Tetratricopeptide repeat (TPR)-like superfamily protein | ClassIIIB |
| At4g21780 | unknown protein; Has 30201 Blast hits to 17322 proteins in 780 species: Archae - 12; | ClassIIIB |
| | Bacteria - 1396; Metazoa - 17338; Fungi - 3422; Plants - 5037; Viruses - 0; Other Eukaryotes - | |
| | 2996 (source: NCBI BLink). | |
| At4g22470 | protease inhibitor/seed storage/lipid transfer protein (LTP) family protein | ClassIIIB |
| At5g60800 | Heavy metal transport/detoxification superfamily protein | ClassIIIB |
| At4g34320 | Protein of unknown function (DUF677) | ClassIIIB |
| At2g47130 | NAD(P)-binding Rossmann-fold superfamily protein | ClassIIIB |
| At5g65600 | Concanavalin A-like lectin protein kinase family protein | ClassIIIB |
| At1g17370 | UBP1B, oligouridylate binding protein 1B | ClassIIIB |
| At1g28390 | Protein kinase superfamily protein | ClassIIIB |
| At4g36900 | DEAR4, RAP2.10, related to AP2 10 | ClassIIIB |
| At2g35910 | RING/U-box superfamily protein | ClassIIIB |
| At5g44990 | Glutathione S-transferase family protein | ClassIIIB |
| At4g31780 | MGD1, MGDA, monogalactosyl diacylglycerol synthase 1 | ClassIIIB |
| At5g51190 | Integrase-type DNA-binding superfamily protein | ClassIIIB |
| At4g23010 | ATUTR2, UTR2, UDP-galactose transporter 2 | ClassIIIB |
| At5g10400 | Histone superfamily protein | ClassIIIB |
| At4g02330 | ATPMEPCRB, Plant invertase/pectin methylesterase inhibitor superfamily | ClassIIIB |
| At2g34930 | disease resistance family protein/LRR family protein | ClassIIIB |
| At2g43000 | anac042, NAC042, NAC domain containing protein 42 | ClassIIIB |
| At5g58110 | chaperone binding; ATPase activators | ClassIIIB |
| At1g14480 | Ankyrin repeat family protein | ClassIIIB |
| At1g17750 | AtPEPR2, PEPR2, PEP1 receptor 2 | ClassIIIB |
| At5g62630 | HIPL2, hipl2 protein precursor | ClassIIIB |
| At5g51390 | unknown protein; Has 30201 Blast hits to 17322 proteins in 780 species: Archae - 12; | ClassIIIB |
| | Bacteria - 1396; Metazoa - 17338; Fungi - 3422; Plants - 5037; Viruses - 0; Other Eukaryotes - | |
| | 2996 (source: NCBI BLink). | |
| At5g07860 | HXXXD-type acyl-transferase family protein | ClassIIIB |
| At4g38000 | DOF4.7, DNA binding with one finger 4.7 | ClassIIIB |
| At2g39900 | GATA type zinc finger transcription factor family protein | ClassIIIB |
| At3g29670 | HXXXD-type acyl-transferase family protein | ClassIIIB |
| At2g17120 | LYM2, lysm domain GPI-anchored protein 2 precursor | ClassIIIB |
| At1g52200 | PLAC8 family protein | ClassIIIB |
| At2g39110 | Protein kinase superfamily protein | ClassIIIB |
| At1g55920 | ATSERAT2; 1, SAT1, SAT5, SERAT2; 1, serine acetyltransferase 2; 1 | ClassIIIB |
| At4g01700 | Chitinase family protein | ClassIIIB |
| At2g31880 | EVR, SOBIR1, Leucine-rich repeat protein kinase family protein | ClassIIIB |
| At3g62720 | ATXT1, XT1, XXT1, xylosyltransferase 1 | ClassIIIB |
| At2g26380 | Leucine-rich repeat (LRR) family protein | ClassIIIB |
| At2g47140 | NAD(P)-binding Rossmann-fold superfamily protein | ClassIIIB |
| At2g19570 | AT-CDA1, CDA1, DESZ, cytidine deaminase 1 | ClassIIIB |
| At3g14360 | alpha/beta-Hydrolases superfamily protein | ClassIIIB |
| At2g37940 | AtIPCS2, Arabidopsis Inositol phosphorylceramide synthase 2 | ClassIIIB |
| At5g60680 | Protein of unknown function, DUF584 | ClassIIIB |
| At5g41680 | Protein kinase superfamily protein | ClassIIIB |
| At3g47380 | Plant invertase/pectin methylesterase inhibitor superfamily protein | ClassIIIB |
| At5g62390 | ATBAG7, BAG7, BCL-2-associated athanogene 7 | ClassIIIB |
| At1g07520 | GRAS family transcription factor | ClassIIIB |
| At4g39030 | EDS5, SID1, MATE efflux family protein | ClassIIIB |
| At3g53130 | CYP97C1, LUT1, Cytochrome P450 superfamily protein | ClassIIIB |
| At1g77030 | hydrolases, acting on acid anhydrides, in phosphorus-containing anhydrides; ATP-dependent | ClassIIIB |
| | helicases; nucleic acid binding; ATP binding; RNA binding; helicases | |
| At3g22160 | VQ motif-containing protein | ClassIIIB |
| At2g42430 | ASL18, LBD16, lateral organ boundaries-domain 16 | ClassIIIB |
| At3g61900 | SAUR-like auxin-responsive protein family | ClassIIIB |
| At5g66070 | RING/U-box superfamily protein | ClassIIIB |
| At2g22750 | basic helix-loop-helix (bHLH) DNA-binding superfamily protein | ClassIIIB |
| At1g02400 | ATGA2OX4, ATGA2OX6, DTA1, GA2OX6, gibberellin 2-oxidase 6 | ClassIIIB |
| At1g51915 | cryptdin protein-related | ClassIIIB |
| At4g19960 | ATKUP9, HAK9, KT9, KUP9, K+ uptake permease 9 | ClassIIIB |
| At4g31000 | Calmodulin-binding protein | ClassIIIB |
| At2g26560 | PLA IIA, PLA2A, PLP2, PLP2, phospholipase A 2A | ClassIIIB |
| At5g10750 | Protein of unknown function (DUF1336) | ClassIIIB |
| At3g55950 | ATCRR3, CCR3, CRINKLY4 related 3 | ClassIIIB |
| At3g50760 | GATL2, galacturonosyltransferase-like 2 | ClassIIIB |
| At4g29670 | ACHT2, atypical CYS HIS rich thioredoxin 2 | ClassIIIB |
| At2g37810 | Cysteine/Histidine-rich C1 domain family protein | ClassIIIB |
| At3g52430 | ATPAD4, PAD4, alpha/beta-Hydrolases superfamily protein | ClassIIIB |
| At1g36640 | unknown protein; FUNCTIONS IN: molecular_function unknown; INVOLVED IN: | ClassIIIB |
| | biological_process unknown; LOCATED IN: endomembrane system; EXPRESSED IN: | |
| | sperm cell, root; BEST Arabidopsis thaliana protein match is unknown protein | |
| | (TAIR: AT1G36622.1); Has 14 Blast hits to 14 proteins in 2 species: Archae - 0; Bacteria - 0; | |
| | Metazoa - 0; Fungi - 0; Plants - 14; Viruses - 0; Other Eukaryotes - 0 (source: NCBI BLink). | |
| At2g20150 | unknown protein; Has 5 Blast hits to 5 proteins in 1 species: Archae - 0; Bacteria - 0; | ClassIIIB |
| | Metazoa - 0; Fungi - 0; Plants - 5; Viruses - 0; Other Eukaryotes - 0 (source: NCBI BLink). | |
| At3g08710 | ATH9, TH9, TRX H9, thioredoxin H-type 9 | ClassIIIB |
| At3g02800 | Tyrosine phosphatase family protein | ClassIIIB |
| At2g24180 | CYP71B6, cytochrome p450 71b6 | ClassIIIB |
| At2g27690 | CYP94C1, cytochrome P450, family 94, subfamily C, polypeptide 1 | ClassIIIB |
| At5g46710 | PLATZ transcription factor family protein | ClassIIIB |
| At3g02790 | zinc finger (C2H2 type) family protein | ClassIIIB |
| At3g53280 | CYP71B5, cytochrome p450 71b5 | ClassIIIB |
| At5g62350 | Plant invertase/pectin methylesterase inhibitor superfamily protein | ClassIIIB |
| At5g40010 | AATP1, AAA-ATPase 1 | ClassIIIB |
| At5g38210 | Protein kinase family protein | ClassIIIB |
| At2g21560 | unknown protein; BEST Arabidopsis thaliana protein match is unknown protein | ClassIIIB |
| | (TAIR: AT4G39190.1); Has 3685 Blast hits to 2305 proteins in 270 species: Archae - 0; | |
| | Bacteria - 156; Metazoa - 1145; Fungi - 322; Plants - 177; Viruses - 6; Other Eukaryotes - | |
| | 1879 (source: NCBI BLink). | |
| At1g59910 | Actin-binding FH2 (formin homology 2) family protein | ClassIIIB |
| At5g58120 | Disease resistance protein (TIR-NBS-LRR class) family | ClassIIIB |
| At5g59480 | Haloacid dehalogenase-like hydrolase (HAD) superfamily protein | ClassIIIB |
| At3g01820 | P-loop containing nucleoside triphosphate hydrolases superfamily protein | ClassIIIB |
| At1g63480 | AT hook motif DNA-binding family protein | ClassIIIB |
| At3g04630 | WDL1, WVD2-like 1 | ClassIIIB |
| At2g17220 | Protein kinase superfamily protein | ClassIIIB |
| At1g16380 | ATCHX1, CHX1, Cation/hydrogen exchanger family protein | ClassIIIB |
| At1g61370 | S-locus lectin protein kinase family protein | ClassIIIB |
| At3g09405 | Pectinacetylesterase family protein | ClassIIIB |
| At3g47550 | RING/FYVE/PHD zinc finger superfamily protein | ClassIIIB |
| At3g59900 | ARGOS, auxin-regulated gene involved in organ size | ClassIIIB |
| At1g24150 | ATFH4, FH4, formin homologue 4 | ClassIIIB |
| At2g16870 | Disease resistance protein (TIR-NBS-LRR class) family | ClassIIIB |
| At2g42350 | RING/U-box superfamily protein | ClassIIIB |
| At5g66620 | DAR6, DA1-related protein 6 | ClassIIIB |
| At4g33960 | unknown protein; FUNCTIONS IN: molecular_function unknown; INVOLVED IN: | ClassIIIB |
| | biological_process unknown; LOCATED IN: endomembrane system; EXPRESSED IN: 20 | |
| | plant structures; EXPRESSED DURING: 10 growth stages; BEST Arabidopsis thaliana | |
| | protein match is: unknown protein (TAIR: AT2G15830.1); Has 32 Blast hits to 32 proteins in | |
| | 4 species: Archae - 0; Bacteria - 0; Metazoa - 0; Fungi - 0; Plants - 32; Viruses - 0; Other | |
| | Eukaryotes - 0 (source: NCBI BLink). | |
| At3g16030 | CES101, lectin protein kinase family protein | ClassIIIB |
| At5g22690 | Disease resistance protein (TIR-NBS-LRR class) family | ClassIIIB |
| At1g11310 | ATMLO2, MLO2, PMR2, Seven transmembrane MLO family protein | ClassIIIB |
| At1g59850 | ARM repeat superfamily protein | ClassIIIB |
| At2g21120 | Protein of unknown function (DUF803) | ClassIIIB |
| At1g05710 | basic helix-loop-helix (bHLH) DNA-binding superfamily protein | ClassIIIB |
| At1g71450 | Integrase-type DNA-binding superfamily protein | ClassIIIB |
| At4g37180 | Homeodomain-like superfamily protein | ClassIIIB |
| At1g61560 | ATMLO6, MLO6, Seven transmembrane MLO family protein | ClassIIIB |
| At5g39710 | EMB2745, Tetratricopeptide repeat (TPR)-like superfamily protein | ClassIIIB |
| At1g05055 | ATGTF2H2, GTF2H2, general transcription factor II H2 | ClassIIIB |
| At3g03660 | WOX11, WUSCHEL related homeobox 11 | ClassIIIB |
| At5g09980 | PROPEP4, elicitor peptide 4 precursor | ClassIIIB |
| At2g26190 | calmodulin-binding family protein | ClassIIIB |
| At3g54200 | Late embryogenesis abundant (LEA) hydroxyproline-rich glycoprotein family | ClassIIIB |
| At1g53440 | Leucine-rich repeat transmembrane protein kinase | ClassIIIB |
| At5g60250 | zinc finger (C3HC4-type RING finger) family protein | ClassIIIB |
| At1g63830 | PLAC8 family protein | ClassIIIB |
| At3g08760 | ATSIK, Protein kinase superfamily protein | ClassIIIB |
| At5g66640 | DAR3, DA1-related protein 3 | ClassIIIB |
| At5g53130 | ATCNGC1, CNGC1, cyclic nucleotide gated channel 1 | ClassIIIB |
| At3g28580 | P-loop containing nucleoside triphosphate hydrolases superfamily protein | ClassIIIB |
| At4g15120 | VQ motif-containing protein | ClassIIIB |
| At2g24600 | Ankyrin repeat family protein | ClassIIIB |
| At2g01450 | ATMPK17, MPK17, MAP kinase 17 | ClassIIIB |
| At1g65690 | Late embryogenesis abundant (LEA) hydroxyproline-rich glycoprotein family | ClassIIIB |
| At1g53920 | GLIP5, GDSL-motif lipase 5 | ClassIIIB |
| At2g38870 | Serine protease inhibitor, potato inhibitor I-type family protein | ClassIIIB |
| At2g40180 | ATHPP2C5, PP2C5, phosphatase 2C5 | ClassIIIB |
| At5g04720 | ADR1-L2, ADR1-like 2 | ClassIIIB |
| At1g72060 | serine-type endopeptidase inhibitors | ClassIIIB |
| At5g24620 | Pathogenesis-related thaumatin superfamily protein | ClassIIIB |
| At2g19190 | FRK1, FLG22-induced receptor-like kinase 1 | ClassIIIB |
| At4g14630 | GLP9, germin-like protein 9 | ClassIIIB |

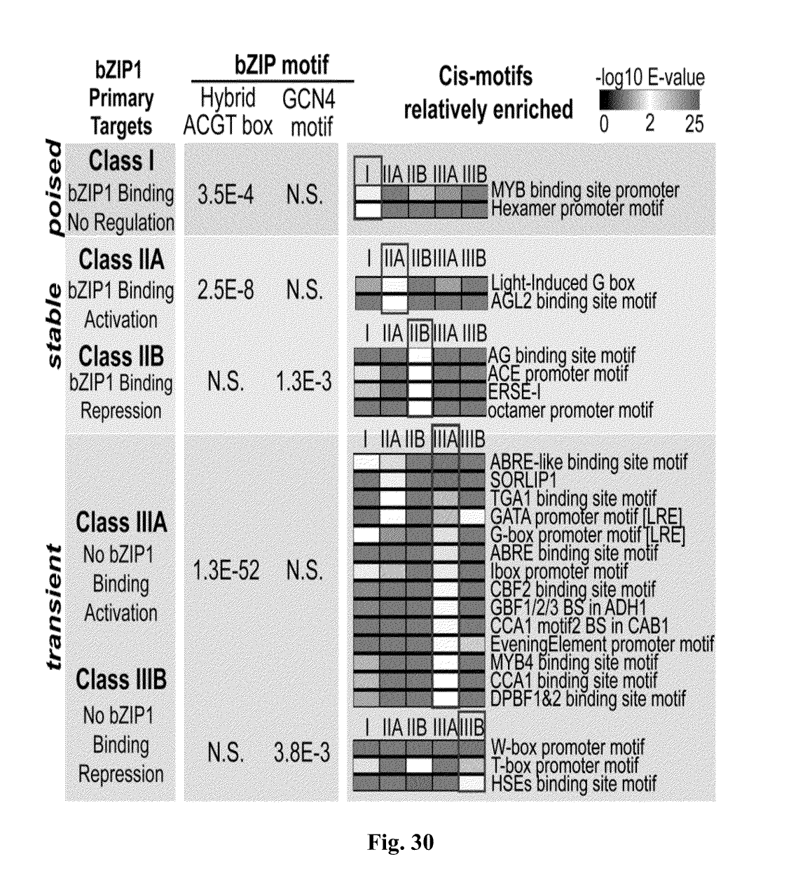

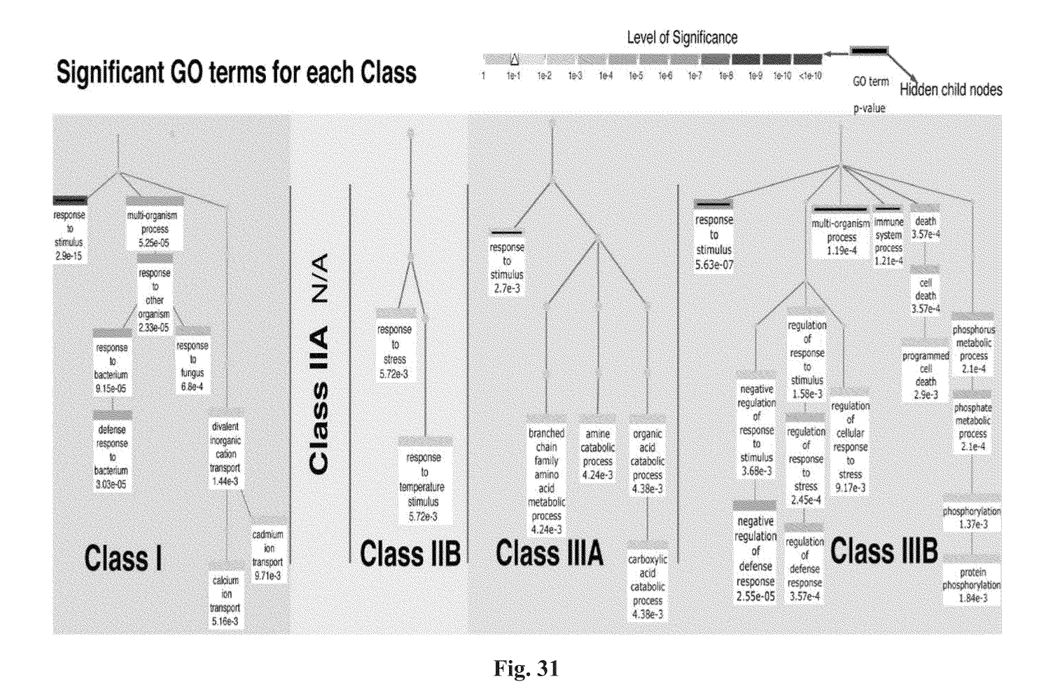

To next explore the biological relevance of the three distinct classes of primary bZIP1 targets, the following features were examined: (1) enrichment of cis-regulatory elements (FIG. 30); (2) comparison to bZIP1 regulated genes in planta (FIG. 29B), and (3) biological relevance to N-signal transduction in isolated cells (FIGS. 29A & 29C) and in planta (FIG. 29C). This comparative analysis uncovered features common to all three classes of bZIP1 targets, as well as specific features of Class III transient targets that are uniquely relevant to rapid N-signal propagation. The features shared by all three classes of bZIP1 primary targets are: i) bZIP1-binding sites: all three classes of genes deemed to be bZIP1 primary targets share enrichment of known bZIP1 binding sites in their promoters (E<0.01, FIG. 30). ii) In planta relevance to bZIP1: all three classes of bZIP1 primary targets identified in the cell-based TARGET system were validated by their significant overlap with bZIP1-regulated genes identified in transgenic plants, either by comparison to a 35S::bZIP1 overexpression line (100/449 genes; 22% overlap; p-val<0.001) or a T-DNA insertion mutant in bZIP1 (89/488 genes; 18.2% overlap; p-val<0.001) (Kang et al., 2010, Molecular Plant 3:361-373) (FIG. 29B). iii) N-regulation in planta: bZIP1 was predicted to be a master regulator in N-response (Gutierrez et al., 2008, Proc. Natl. Acad. Sci. U.S.A. 105:4939-4944; Obertello et al., 2010, BMC systems biology 4:111), and in support of this, all three classes of bZIP1 primary targets in protoplasts are significantly enriched with N-responsive genes in planta (Krouk et al., 2010, Genome Biology 11:R123; Gutierrez et al., 2008, Proc. Natl. Acad. Sci. U.S.A. 105:4939-4944; Wang et al., 2003, Plant Physiol. 132(2):556-567; Wang et al., 2004, Plant physiology 136(1):2512-2522) (438/1,308 genes, p-val<0.001) (FIG. 29C). iv) known bZIP1 functions: all three classes of targets show enrichment of GO-terms associated with other known bZIP1 functions (e.g. Stimulus/Stress) (FIG. 31). Specifically, bZIP1 is reported as a master regulator in response to darkness and sugar starvation (Baena-Gonzalez et al., 2007, Nature 448:938; Kang et al., 2010, Molecular Plant 3:361-373). Consistent with this, all three classes of bZIP1 primary targets share a significant overlap (p-val<0.001) with genes induced by sugar starvation and extended darkness (Krouk et al., 2009, PLoS Comput Biol 5(3):e1000326).

In addition to these common features consistent with the role of bZIP1 in planta (Baena-Gonzalez et al., 2007, Nature 448:938; Kang et al., 2010, Molecular Plant 3:361-373; Gutierrez et al., 2008, Proc. Natl. Acad. Sci. U.S.A. 105:4939-4944), distinctive features for the Class III transient bZIP1 primary targets specifically relevant to rapid N-signaling were uncovered. These class-specific features are outlined below.

Class I “Poised” Targets (TF Binding Only).

Class I bZIP1 primary targets (407 genes) that are bound, but not regulated by bZIP1, are significantly enriched in genes involved in response to biotic/abiotic stimuli, and transport of divalent ions (FDR<0.01) (FIG. 29A; FIG. 31). They are also significantly enriched in the known bZIP1 binding site “hybrid ACGT box” (E=3.5e-4), supporting that they are valid primary targets of bZIP1 (FIG. 30). This suggests that bZIP1 is bound to and poised to activate these target genes, possibly in response to a signal or a TF partner not present in the experimental conditions.

Class II “Stable” Targets (TF Binding and Regulation).

Class II targets (120 genes) are regulated and bound by bZIP1. This 23% overlap (p-val<0.001) between transcriptome and ChIP-Seq data (FIG. 29A), is comparable to the relatively low overlap observed for other TF perturbation studies performed in planta [23% ABI3 (Monke et al., 2012, Nucleic Acids Research 40:82401); 5% ASR5 (Arenhart et al., 2014, Molecular plant 7(4):709-721); KNOTTED1 20%-30% (Bolduc et al., 2012, Gene Dev 26(15):1685-1690)] and in other eukaryotes [8% BRCA1 (Gorski et al, 2011, Nucleic Acids Research 39(22):9536-9548); LRH-1 32% (Bianco et al., 2014, Cancer research 74(7):2015-2025)]. Thus, the Class II “stable” bZIP1 targets correspond to the “gold standard” set typically identified in TF studies across eukaryotes (Gorski et al, 2011, Nucleic Acids Research 39(22):9536-9548; Hughes et al., 2013, Genetics 195(1):9-36; Monke et al., 2012, Nucleic Acids Research 40:82401; Arenhart et al., 2014, Molecular plant 7(4):709-721; Bolduc et al., 2012, Gene Dev 26(15):1685-1690; Bianco et al., 2014, Cancer research 74(7):2015-2025). Further, the cis-element analysis suggests the novel finding that bZIP1 functions to activate or repress target gene expression via two distinct binding sites (FIG. 30). The targets activated by bZIP1 (Class IIA), are significantly enriched with the hybrid ACGT box bZIP1 binding site (E=2.5e-8) (FIG. 30). By contrast, genes repressed by bZIP1 (Class IIB) are enriched with the bZIP binding site GCN4 (E=1.3e-3) (FIG. 30). Interestingly, the GCN4 motif was reported to mediate N and amino acid starvation sensing in yeast (Hill et al., 1986, Science 234:451-457), suggesting a conserved link between bZIPs and nutrient sensing across eukaryotes. Finally, Class II targets share the “Stimulus/Stress” GO terms with other classes, but surprisingly, no significant biological terms unique to Class II targets were identified (FIG. 29A and FIG. 31).

Class III “Transient” Targets (TF Regulation, but No Detectable TF Binding).

Unexpectedly, the largest group of bZIP1 primary targets (781 genes), is represented by the Class III “transient” targets i.e., primary targets regulated by bZIP1 perturbation but not detectably bound by it (FIG. 29A). Paradoxically, Class IIIA “transient” targets that are activated by bZIP1 are the most significantly enriched in the known bZIP1 binding site (E=1.3e-52) (FIG. 30), despite their lack of detectable bZIP1 binding. Class IIIB targets repressed by bZIP1 are significantly enriched in a distinct bZIP binding site “GCN4” (E=3.8e-3) (FIG. 30). Intriguingly, both of these known bZIP1-binding sites in the Class III transient genes are also observed in the Class II stable target genes (TF-bound and regulated) (FIG. 30). The lack of detectable TF-binding for Class III targets likely represents a transient or weak interaction of bZIP1 and these primary targets, rather than an indirect interaction, as the ChIP-Seq protocol can also detect indirect binding (e.g. via interacting TF partners). The trivial explanation that the mRNAs for Class IIIA genes are stabilized by CHX or bZIP1 is not supported by the data, as the CHX effect was accounted for by filtering out genes whose response to DEX-induced nuclear localization of bZIP1 is altered by CHX-treatment. Instead, the Class III primary targets likely represent a transient interaction between bZIP1 and its targets. Indeed, 41 genes from Class III transient targets have detectable bZIP1 binding at one or more of the earlier time-points (1, 5, 30, 60 min) measured by ChIP-Seq, following DEX-induced TF nuclear import (FIG. 29D; Table 20). These Class III transient genes are uniquely relevant to rapid N-signaling, as described below.

TABLE 20
Class III bZIP1-regulated genes that show evidence of bZIP1 binding
at early (1, 5, 30 or 60 min), but not at a 5 hr time point.

| | |
|---|---|
| At4g14368 | Regulator of chromosome condensation (RCC1) family protein |
| At1g10060 | ATBCAT-1, BCAT-1, branched-chain amino acid transaminase 1 |
| At1g18460 | alpha/beta-Hydrolases superfamily protein |
| At3g60690 | SAUR-like auxin-responsive protein family |
| At2g37840 | Protein kinase superfamily protein |
| At3g14780 | CONTAINS InterPro DOMAIN/s: Transposase, Ptta/En/Spm, plant (InterPro:IPR004252); BEST |
| | Arabidopsis thaliana protein match is: glucan synthase-like 4 (TAIR:AT3G14570.2); Has 315 Blast hits |
| | to 313 proteins in 50 species: Archae - 2; Bacteria - 16; Metazoa - 11; Fungi - 7; Plants - 181; Viruses - |
| | 2; Other Eukaryotes - 96 (source: NCBI BLink). |
| At3g01820 | P-loop containing nucleoside triphosphate hydrolases superfamily protein |
| At1g30820 | CTP synthase family protein |
| At1g73240 | CONTAINS InterPro DOMAIN/s: Nucleoporin protein Ndc1-Nup (InterPro:IPR019049); Has 36 Blast |
| | hits to 36 proteins in 17 species: Archae - 0; Bacteria - 0; Metazoa - 1; Fungi - 0; Plants - 35; Viruses - |
| | 0; Other Eukaryotes - 0 (source: NCBI BLink). |
| At4g17140 | pleckstrin homology (PH) domain-containing protein |
| At1g04410 | Lactate/malate dehydrogenase family protein |
| At5g59590 | UGT76E2, UDP-glucosyl transferase 76E2 |
| At1g53430 | Leucine-rich repeat transmembrane protein kinase |
| At1g11000 | ATMLO4, MLO4, Seven transmembrane MLO family protein |
| At1g08090 | ACH1, ATNRT2.1, ATNRT2:1, LIN1, NRT2, NRT2.1, NRT2:1, NRT2;1AT, nitrate transporter 2:1 |
| At1g08830 | CSD1, copper/zinc superoxide dismutase 1 |
| At3g02150 | PTF1, TCP13, TFPD, plastid transcription factor 1 |
| At5g24430 | Calcium-dependent protein kinase (CDPK) family protein |
| At3g51840 | ACX4, ATG6, ATSCX, acyl-CoA oxidase 4 |
| At1g06570 | HPD, PDS1, phytoene desaturation 1 |
| At4g19810 | Glycosyl hydrolase family protein with chitinase insertion domain |
| At5g01590 | unknown protein; FUNCTIONS IN: molecular_function unknown; INVOLVED IN: |
| | biological_process unknown; LOCATED IN: chloroplast, chloroplast envelope; EXPRESSED IN: 22 |
| | plant structures; EXPRESSED DURING: 13 growth stages; Has 60 Blast hits to 59 proteins in 31 |
| | species: Archae - 0; Bacteria - 20; Metazoa - 1; Fungi - 2; Plants - 33; Viruses - 0; Other Eukaryotes - 4 |
| | (source: NCBI BLink). |
| At1g77030 | hydrolases, acting on acid anhydrides, in phosphorus-containing anhydrides;ATP-dependent |
| | helicases;nucleic acid binding;ATP binding;RNA binding;helicases |
| At3g15950 | NAI2, DNA topoisomerase-related |
| At5g43430 | ETFBETA, electron transfer flavoprotein beta |
| At4g34180 | Cyclase family protein |
| At1g19220 | ARF11, ARF19, IAA22, auxin response factor 19 |
| At1g08630 | THA1, threonine aldolase 1 |
| At1g67510 | Leucine-rich repeat protein kinase family protein |
| At4g38340 | Plant regulator RWP-RK family protein (NLP3) |
| At1g57560 | AtMYB50, MYB50, myb domain protein 50 |
| At4g38500 | Protein of unknown function (DUF616) |
| At5g53130 | ATCNGC1, CNGC1, cyclic nucleotide gated channel 1 |
| At1g03090 | MCCA, methylcrotonyl-CoA carboxylase alpha chain, mitochondrial/3-methylcrotonyl-CoA |
| | carboxylase 1 (MCCA) |
| At1g44100 | AAP5, amino acid permease 5 |
| At3g61850 | DAG1, Dof-type zinc finger DNA-binding family protein |
| At1g18270 | ketose-bisphosphate aldolase class-II family protein |
| At1g26730 | EXS (ERD1/XPR1/SYG1) family protein |
| At5g46710 | PLATZ transcription factor family protein |
| At3g48850 | PHT3;2, phosphate transporter 3;2 |
| At2g02700 | Cysteine/Histidine-rich C1 domain family protein |

The Class III Transient bZIP1 Primary Targets Comprise “First Responders” in Rapid N-Signaling.

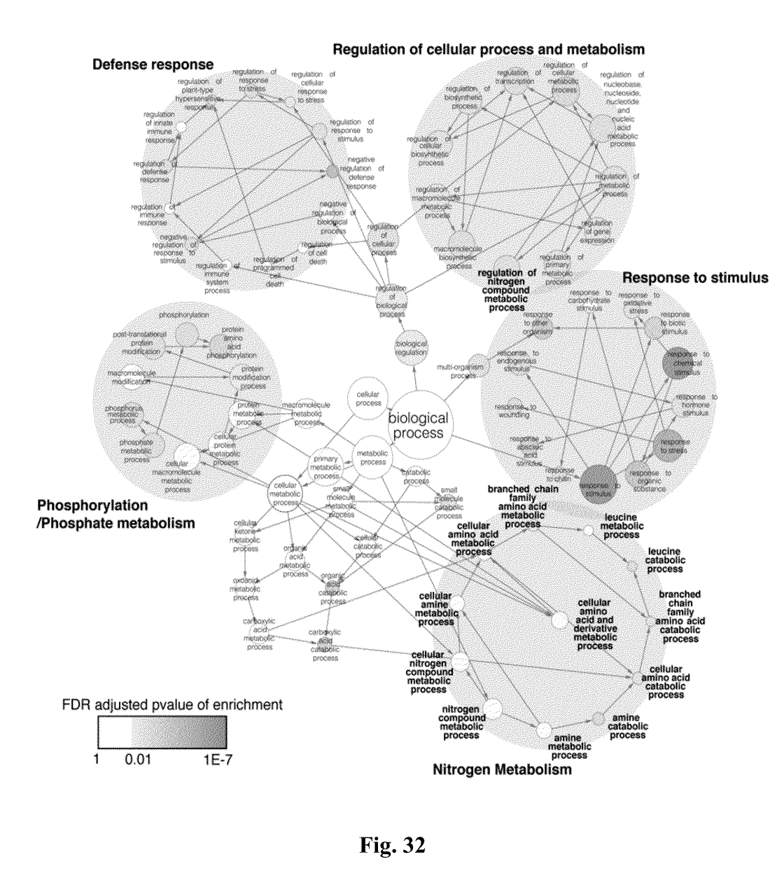

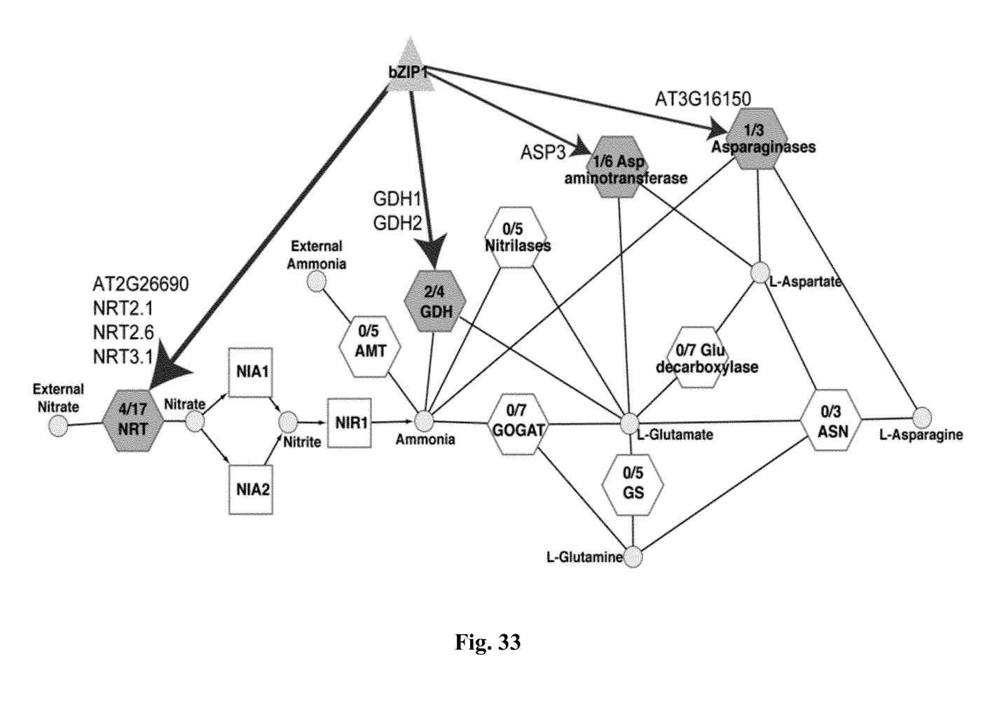

In line with its role as a master regulator in a N-response gene network, all three classes of bZIP1 primary targets uncovered in this cell-based study are significantly enriched with N-responsive genes observed in whole plants (Krouk et al., 2010, Genome Biology 11(12):R123; Gutierrez et al., 2008, Proc. Natl. Acad. Sci. U.S.A. 105:4939-4944; Wang et al., 2003, Plant Physiol. 132(2):556-567; Wang et al., 2004, Plant physiology 136(1):2512-2522) (FIG. 29C; overlap with the “union” of N-responsive genes in planta). Unexpectedly, the “transient” Class III bZIP1 targets—regulated by, but not stably bound to bZIP1—are uniquely relevant to rapid and dynamic N-signaling in planta (FIG. 29C). This conclusion is based on the following evidence: First, the Class IIIA transient bZIP1 targets have the largest and most significant overlap (p-val<0.001; FIG. 29C) with the 147 genes induced by N-signals in this cell-based TARGET study (Table 12). Second, only Class III transient bZIP1 targets have a significant enrichment in genes involved in N-related biological processes (enrichment of GO terms p-val<0.01) including amino acid metabolism (FIG. 29A; FIG. 32; Table 21), a role also supported by in planta studies of bZIP1 (Dietrich et al., 2011, The Plant Cell 23:381-395). Third, the Class III transient genes comprise the bulk of the bZIP1 targets in the N-assimilation pathway (FIG. 33 & Table 22), including the “early N-responders”, such as the high-affinity nitrate transporter, NRT2.1, induced rapidly (<12 minutes) and transiently following N-signal perturbation in planta (Krouk et al., 2010, Genome Biology 11(12):R123). Fourth, the Class III transient targets exclusively comprise all of the genes regulated by a N-treatment×bZIP1 interaction (28 genes) (FIG. 29C; FIG. 28). These include well-known early mediators of N-signaling induced at 6-12 min after N-provision (Krouk et al., 2010, Genome Biology 11(12):R123), including the NIN-like transcription factor 3 (NLP3; At4g38340) (Konishi et al., 2013, Nature Communications 4: 1617), and the LBD39 transcription factor (At4g37540) (Rubin et al., 2009, The Plant Cell 21(11):3567-3584). NLP3 belongs to the NIN-like transcription factor family which plays an essential role in nitrate signaling (Konishi et al., 2013, Nature Communications 4: 1617). In this study, NLP3 is a transient bZIP1 target whose up-regulation by bZIP1 is dependent on the N-signal (FIG. 28; Table 17). LBD39, which has been reported to fine-tune the magnitude of the N-response in planta (Rubin et al., 2009, The Plant Cell 21(11):3567-3584), is a transient bZIP1 target that is only induced by bZIP1 in the presence of the N-signal in this cell-based study (FIG. 28; Table 17). This N-signal×bZIP1 interaction could be a post-translational modification of bZIP1, reminiscent of its post-translational modification in response to other abiotic signals (e.g. sugar and stress signals) (Dietrich et al., 2011, The Plant Cell 23:381-395). The N-signal×bZIP1 interaction could also involve translational/transcriptional effects of the N-signal on its interacting TF partners, as depicted in FIG. 24B.

TABLE 21
Significantly over-represented GO terms (FDR adjusted p-val < 0.01) identified for genes in each of the five subclasses of bZIP1 targets. (Nitrogen related biological processes are in bold)

| | GO ID | Term | Observed Frequency | Expected Frequency | p-value | Genes |
|---|---|---|---|---|---|---|
| ClassI | GO:0006950 | response to | 86 out of 275 | 2104 out of 15002 | 1.69E−10 | AT5G49480\|AT1G17870\|AT1G80850\|AT4G23190\|AT5G06320\|AT3G09 |
| | | stress | genes, 31.3% | genes, 14% | | 440\|AT4G17615\|AT2G40000\|AT5G47230\|AT3G17390\|AT1G55450\|AT3 |
| | | | | | | G50980\|AT1G27760\|AT5G15090\|AT1G42560\|AT3G52930\|AT3G23250\| |
| | | | | | | AT1G09080\|AT2G26690\|AT3G06510\|AT5G02020\|AT1G74310\|AT2G30 |
| | | | | | | 250\|AT1G19020\|AT4G05100\|AT2G05710\|AT1G68760\|AT3G44260\|AT1 |
| | | | | | | G32920\|AT3G52450\|AT2G41430\|AT3G22370\|AT1G01060\|AT5G14740\| |
| | | | | | | AT1G78080\|AT3G13790\|AT3G10920\|AT3G15500\|AT2G24570\|AT5G59 |
| | | | | | | 820\|AT1G19180\|AT3G19580\|AT1G22070\|AT2G43130\|AT1G05680\|AT1 |
| | | | | | | G45145\|AT2G40140\|AT2G32120\|AT2G03760\|AT1G42990\|AT5G63790\| |
| | | | | | | AT4G39090\|AT5G45110\|AT5G64905\|AT3G49530\|AT1G01720\|AT1G76 |
| | | | | | | 180\|AT5G39580\|AT1G59870\|AT3G51920\|AT5G06290\|AT5G61890\|AT1 |
| | | | | | | G62300\|AT3G55440\|AT4G01370\|AT2G46830\|AT2G17840\|AT1G73080\| |
| | | | | | | AT5G58070\|AT4G39640\|AT3G10985\|AT1G20440\|AT1G10170\|AT4G37 |
| | | | | | | 010\|AT4G33950\|AT5G62530\|AT2G35930\|AT1G29395\|AT1G33590\|AT3 |
| | | | | | | G50970\|AT5G37500\|AT1G20450\|AT4G20830\|AT1G32640\|AT4G39080\| |
| | | | | | | AT1G71697 |
| | GO:0009628 | response to | 66 out of 275 | 1360 out of 15002 | 1.69E−10 | AT5G49480\|AT1G17870\|AT2G43130\|AT1G05680\|AT3G09440\|AT2G40 |
| | | abiotic | genes, 24% | genes, 9.1% | | 140\|AT4G27280\|AT4G17615\|AT2G32120\|AT2G03760\|AT5G47230\|AT3 |
| | | stimulus | | | | G17390\|AT1G56590\|AT1G55450\|AT3G50980\|AT1G27760\|AT4G39090\| |
| | | | | | | AT3G49530\|AT1G76180\|AT3G51920\|AT5G05600\|AT5G06290\|AT3G55 |
| | | | | | | 440\|AT4G01370\|AT2G46830\|AT3G52930\|AT1G61890\|AT4G37370\|AT2 |
| | | | | | | G17840\|AT5G58070\|AT3G23250\|AT1G09080\|AT3G06510\|AT5G02020\| |
| | | | | | | AT1G74310\|AT1G20440\|AT2G30250\|AT2G47000\|AT4G05100\|AT1G10 |
| | | | | | | 170\|AT4G33950\|AT1G78290\|AT5G62530\|AT2G05710\|AT2G35930\|AT1 |
| | | | | | | G29395\|AT1G33590\|AT3G50970\|AT4G37270\|AT3G52450\|AT5G37500\| |
| | | | | | | AT3G62410\|AT2G41430\|AT1G20450\|AT3G22370\|AT1G80010\|AT1G01 |
| | | | | | | 060\|AT1G32640\|AT1G78080\|AT3G13790\|AT4G39080\|AT3G10920\|AT3 |
| | | | | | | G15500\|AT5G59820\|AT5G01500\|AT3G19580 |
| | GO:0042221 | response to | 79 out of 275 | 1892 out of 15002 | 5.36E−10 | AT1G22070\|AT1G17870\|AT4G23190\|AT1G68765\|AT1G05680\|AT3G15 |
| | | chemical | genes, 28.7% | genes, 12.6% | | 210\|AT1G45145\|AT3G16857\|AT3G09440\|AT2G40140\|AT1G43910\|AT4 |
| | | stimulus | | | | G17615\|AT3G53480\|AT2G32120\|AT2G40000\|AT2G03760\|AT1G15080\| |
| | | | | | | AT5G47230\|AT3G08590\|AT1G42990\|AT3G50980\|AT5G63790\|AT4G39 |
| | | | | | | 090\|AT3G49530\|AT4G34160\|AT1G76180\|AT1G59870\|AT3G51920\|AT3 |
| | | | | | | G04730\|AT1G62300\|AT4G08950\|AT3G55440\|AT4G01370\|AT2G46830\| |
| | | | | | | AT5G11670\|AT3G52930\|AT2G17840\|AT5G27420\|AT1G73080\|AT4G39 |
| | | | | | | 640\|AT3G23250\|AT2G26690\|AT1G74310\|AT1G20440\|AT5G02240\|AT2 |
| | | | | | | G25490\|AT1G19020\|AT2G47000\|AT4G05100\|AT1G10170\|AT5G59450\| |
| | | | | | | AT3G46620\|AT4G33950\|AT3G13920\|AT4G37260\|AT2G05710\|AT2G35 |
| | | | | | | 930\|AT1G29395\|AT3G50970\|AT4G37270\|AT3G52450\|AT5G37500\|AT3 |
| | | | | | | G62410\|AT2G41430\|AT1G20450\|AT4G20830\|AT1G01060\|AT1G32640\| |
| | | | | | | AT1G78080\|AT3G10920\|AT3G15500\|AT3G52800\|AT2G24570\|AT4G05 |
| | | | | | | 320\|AT2G04880\|AT5G59820\|AT1G19180\|AT3G19580\|AT2G23320 |
| | GO:0050896 | response to | 121 out of 275 | 3689 out of 15002 | 6.14E−10 | AT5G49480\|AT1G17870\|AT1G80850\|AT4G23190\|AT3G15210\|AT5G06 |
| | | stimulus | genes, 44% | genes, 24.6% | | 320\|AT3G09440\|AT4G27280\|AT4G17615\|AT2G40000\|AT1G15080\|AT5 |
| | | | | | | G47230\|AT3G17390\|AT1G55450\|AT3G50980\|AT1G27760\|AT5G15090\| |
| | | | | | | AT3G04730\|AT4G08950\|AT1G42560\|AT3G52930\|AT5G27420\|AT3G23 |
| | | | | | | 250\|AT1G09080\|AT2G26690\|AT3G06510\|AT5G02020\|AT1G74310\|AT2 |
| | | | | | | G30250\|AT1G19020\|AT2G47000\|AT4G05100\|AT5G59450\|AT3G46620\| |
| | | | | | | AT1G78290\|AT2G05710\|AT1G68760\|AT3G44260\|AT1G32920\|AT3G52 |
| | | | | | | 450\|AT2G41430\|AT3G22370\|AT1G01060\|AT5G14740\|AT1G78080\|AT3 |
| | | | | | | G13790\|AT3G10920\|AT3G15500\|AT4G36010\|AT2G24570\|AT2G04880\| |
| | | | | | | AT5G59820\|AT5G01500\|AT1G19180\|AT3G19580\|AT2G23320\|AT1G22 |
| | | | | | | 070\|AT2G43130\|AT1G05680\|AT1G68765\|AT1G45145\|AT3G16857\|AT2 |
| | | | | | | G40140\|AT1G43910\|AT3G53480\|AT2G32120\|AT2G03760\|AT1G56590\| |
| | | | | | | AT3G08590\|AT1G42990\|AT5G63790\|AT4G39090\|AT5G45110\|AT5G64 |
| | | | | | | 905\|AT3G49530\|AT1G01720\|AT4G34160\|AT1G76180\|AT5G39580\|AT1 |
| | | | | | | G59870\|AT3G51920\|AT5G05600\|AT5G06290\|AT5G61890\|AT1G62300\| |
| | | | | | | AT3G55440\|AT4G01370\|AT2G46830\|AT5G11670\|AT1G61890\|AT4G37 |
| | | | | | | 370\|AT2G17840\|AT1G73080\|AT5G58070\|AT4G39640\|AT3G10985\|AT1 |
| | | | | | | G20440\|AT5G02240\|AT2G25490\|AT1G10170\|AT4G37010\|AT4G33950\| |
| | | | | | | AT3G13920\|AT5G62530\|AT4G37260\|AT2G35930\|AT1G29395\|AT1G33 |
| | | | | | | 590\|AT3G50970\|AT4G37270\|AT5G37500\|AT3G62410\|AT1G20450\|AT4 |
| | | | | | | G20830\|AT1G80010\|AT1G32640\|AT4G39080\|AT1G71697\|AT3G52800\| |
| | | | | | | AT4G05320\|AT1G29690 |
| | GO:0010033 | response to | 57 out of 275 | 1148 out of 15002 | 1.64E−09 | AT1G22070\|AT1G68765\|AT1G05680\|AT3G15210\|AT3G16857\|AT2G40 |
| | | organic | genes, 20.7% | genes, 7.7% | | 140\|AT1G43910\|AT4G17615\|AT3G53480\|AT2G40000\|AT2G03760\|AT1 |
| | | substance | | | | G15080\|AT5G47230\|AT1G42990\|AT3G49530\|AT4G34160\|AT1G76180\| |
| | | | | | | AT1G59870\|AT3G51920\|AT3G04730\|AT1G62300\|AT4G08950\|AT4G01 |
| | | | | | | 370\|AT2G46830\|AT5G27420\|AT1G73080\|AT3G23250\|AT2G26690\|AT1 |
| | | | | | | G20440\|AT5G02240\|AT2G25490\|AT2G47000\|AT4G05100\|AT5G59450\| |
| | | | | | | AT3G46620\|AT4G33950\|AT4G37260\|AT2G05710\|AT2G35930\|AT1G29 |
| | | | | | | 395\|AT3G50970\|AT3G52450\|AT5G37500\|AT3G62410\|AT1G20450\|AT1 |
| | | | | | | G01060\|AT1G32640\|AT1G78080\|AT3G15500\|AT3G52800\|AT2G24570\| |
| | | | | | | AT4G05320\|AT2G04880\|AT5G59820\|AT1G19180\|AT3G19580\|AT2G23 |
| | | | | | | 320 |
| | GO:0010200 | response to | 19 out of 275 | 127 out of 15002 | 2.22E−09 | AT5G27420\|AT1G32640\|AT3G49530\|AT2G40140\|AT4G37260\|AT1G42 |
| | | chitin | genes, 6.9% | genes, 0.8% | | 990\|AT3G19580\|AT5G59450\|AT3G15210\|AT3G46620\|AT5G59820\|AT5 |
| | | | | | | G47230\|AT3G23250\|AT2G35930\|AT3G52800\|AT2G23320\|AT1G62300\| |
| | | | | | | AT2G24570\|AT3G52450 |
| | GO:0009743 | response to | 21 out of 275 | 203 out of 15002 | 8.31E−08 | AT5G27420\|AT3G49530\|AT4G34160\|AT5G59450\|AT3G15210\|AT3G46 |
| | | carbohydrate | genes, 7.6% | genes, 1.4% | | 620\|AT5G59820\|AT5G47230\|AT3G23250\|AT1G62300\|AT3G52450\|AT1 |
| | | stimulus | | | | G32640\|AT2G40140\|AT4G37260\|AT1G42990\|AT3G19580\|AT2G35930\| |
| | | | | | | AT3G52800\|AT3G62410\|AT2G23320\|AT2G24570 |
| | GO:0006970 | response to | 29 out of 275 | 425 out of 15002 | 3.21E−07 | AT5G49480\|AT4G05100\|AT1G10170\|AT1G05680\|AT3G51920\|AT1G01 |
| | | osmotic | genes, 10.5% | genes, 2.8% | | 060\|AT4G33950\|AT5G62530\|AT1G78080\|AT4G39080\|AT3G55440\|AT3 |
| | | stress | | | | G10920\|AT4G01370\|AT2G46830\|AT2G05710\|AT4G17615\|AT3G52930\| |
| | | | | | | AT2G17840\|AT5G58070\|AT2G03760\|AT5G59820\|AT3G23250\|AT1G55 |
| | | | | | | 450\|AT5G02020\|AT1G20440\|AT3G19580\|AT1G27760\|AT2G30250\|AT4 |
| | | | | | | G39090 |
| | GO:0009651 | response to | 28 out of 275 | 397 out of 15002 | 3.21E−07 | AT5G49480\|AT4G05100\|AT1G10170\|AT1G05680\|AT3G51920\|AT1G01 |
| | | salt stress | genes, 10.2% | genes, 2.6% | | 060\|AT4G33950\|AT5G62530\|AT1G78080\|AT4G39080\|AT3G55440\|AT3 |
| | | | | | | G10920\|AT4G01370\|AT2G46830\|AT2G05710\|AT4G17615\|AT3G52930\| |
| | | | | | | AT2G17840\|AT5G58070\|AT2G03760\|AT5G59820\|AT3G23250\|AT1G55 |
| | | | | | | 450\|AT5G02020\|AT3G19580\|AT1G27760\|AT2G30250\|AT4G39090 |
| | GO:0009415 | response to | 20 out of 275 | 211 out of 15002 | 5.96E−07 | AT1G76180\|AT1G05680\|AT3G51920\|AT3G50970\|AT4G33950\|AT3G52 |
| | | water | genes, 7.3% | genes, 1.4% | | 450\|AT1G32640\|AT1G78080\|AT5G37500\|AT1G20440\|AT3G19580\|AT3 |
| | | | | | | G15500\|AT3G50980\|AT2G35930\|AT1G29395\|AT4G39090\|AT4G17615\| |
| | | | | | | AT2G41430\|AT1G20450\|AT2G17840 |
| | GO:0009414 | response to | 19 out of 275 | 202 out of 15002 | 1.46E−06 | AT1G76180\|AT1G05680\|AT3G51920\|AT3G50970\|AT4G33950\|AT3G52 |
| | | water | genes, 6.9% | genes, 1.3% | | 450\|AT1G32640\|AT1G78080\|AT5G37500\|AT1G20440\|AT3G19580\|AT3 |
| | | deprivation | | | | G15500\|AT2G35930\|AT1G29395\|AT4G39090\|AT4G17615\|AT2G41430\| |
| | | | | | | AT1G20450\|AT2G17840 |
| | GO:0009737 | response to | 24 out of 275 | 340 out of 15002 | 3.21E−06 | AT4G05100\|AT1G76180\|AT1G05680\|AT3G15210\|AT1G59870\|AT3G51 |
| | | abscisic | genes, 8.7% | genes, 2.3% | | 920\|AT1G01060\|AT4G33950\|AT1G32640\|AT4G37260\|AT4G01370\|AT2 |
| | | acid | | | | G46830\|AT1G43910\|AT2G05710\|AT1G29395\|AT4G17615\|AT5G27420\| |
| | | stimulus | | | | AT1G15080\|AT3G50970\|AT5G37500\|AT1G20440\|AT3G19580\|AT1G20 |
| | | | | | | 450\|AT5G02240 |
| | GO:0009266 | response to | 25 out of 275 | 399 out of 15002 | 1.33E−05 | AT3G49530\|AT3G22370\|AT1G17870\|AT2G43130\|AT1G76180\|AT5G06 |
| | | temperature | genes, 9.1% | genes, 2.7% | | 290\|AT3G09440\|AT2G40140\|AT4G01370\|AT1G29395\|AT4G17615\|AT2 |
| | | stimulus | | | | G17840\|AT2G32120\|AT5G58070\|AT5G59820\|AT5G47230\|AT3G50970\| |
| | | | | | | AT1G09080\|AT3G17390\|AT3G06510\|AT5G37500\|AT1G74310\|AT1G20 |
| | | | | | | 440\|AT2G30250\|AT1G20450 |
| | GO:0009409 | response to | 19 out of 275 | 269 out of 15002 | 7.22E−05 | AT3G49530\|AT3G22370\|AT1G76180\|AT5G06290\|AT2G40140\|AT4G01 |
| | | cold | genes, 6.9% | genes, 1.8% | | 370\|AT1G29395\|AT4G17615\|AT2G17840\|AT5G58070\|AT5G47230\|AT5 |
| | | | | | | G59820\|AT3G50970\|AT3G17390\|AT3G06510\|AT5G37500\|AT1G20440\| |
| | | | | | | AT2G30250\|AT1G20450 |
| | GO:0009719 | response to | 39 out of 275 | 920 out of 15002 | 8.19E−05 | AT2G47000\|AT4G05100\|AT1G68765\|AT1G05680\|AT3G15210\|AT4G33 |
| | | endogenous | genes, 14.2% | genes, 6.1% | | 950\|AT3G16857\|AT4G37260\|AT1G43910\|AT2G05710\|AT1G29395\|AT4 |
| | | stimulus | | | | G17615\|AT3G53480\|AT1G15080\|AT5G47230\|AT3G50970\|AT5G37500\| |
| | | | | | | AT1G20450\|AT4G34160\|AT1G76180\|AT1G59870\|AT3G51920\|AT3G04 |
| | | | | | | 730\|AT1G01060\|AT4G08950\|AT1G32640\|AT1G78080\|AT3G15500\|AT4 |
| | | | | | | G01370\|AT2G46830\|AT5G27420\|AT1G73080\|AT3G23250\|AT2G26690\| |
| | | | | | | AT1G19180\|AT1G20440\|AT3G19580\|AT5G02240\|AT2G25490 |
| | GO:0042742 | defense | 15 out of 275 | 201 out of 15002 | 0.000464 | AT1G22070\|AT2G40000\|AT5G15090\|AT1G10170\|AT4G23190\|AT5G06 |
| | | response to | genes, 5.5% | genes, 1.3% | | 320\|AT1G59870\|AT5G06290\|AT4G33950\|AT5G14740\|AT1G19180\|AT3 |
| | | bacterium | | | | G10920\|AT4G39090\|AT2G24570\|AT5G45110 |
| | GO:0009725 | response to | 35 out of 275 | 849 out of 15002 | 0.000474 | AT2G47000\|AT4G05100\|AT1G68765\|AT1G05680\|AT3G15210\|AT4G33 |
| | | hormone | genes, 12.7% | genes, 5.7% | | 950\|AT3G16857\|AT4G37260\|AT1G43910\|AT2G05710\|AT1G29395\|AT4 |
| | | stimulus | | | | G17615\|AT3G53480\|AT1G15080\|AT5G47230\|AT3G50970\|AT5G37500\| |
| | | | | | | AT1G20450\|AT4G34160\|AT1G76180\|AT1G59870\|AT3G51920\|AT3G04 |
| | | | | | | 730\|AT1G01060\|AT4G08950\|AT1G32640\|AT1G78080\|AT4G01370\|AT2 |
| | | | | | | G46830\|AT5G27420\|AT3G23250\|AT1G20440\|AT3G19580\|AT5G02240\| |
| | | | | | | AT2G25490 |
| | GO:0009607 | response to | 28 out of 275 | 610 out of 15002 | 0.000597 | AT1G22070\|AT1G10170\|AT4G23190\|AT5G06320\|AT4G33950\|AT1G45 |
| | | biotic | genes, 10.2% | genes, 4.1% | | 145\|AT2G40140\|AT3G44260\|AT2G40000\|AT3G50970\|AT1G42990\|AT4 |
| | | stimulus | | | | G39090\|AT2G41430\|AT5G45110\|AT3G49530\|AT5G15090\|AT5G39580\| |
| | | | | | | AT1G59870\|AT5G06290\|AT5G61890\|AT5G14740\|AT3G10920\|AT4G36 |
| | | | | | | 010\|AT4G01370\|AT2G24570\|AT3G10985\|AT1G19180\|AT1G20440 |
| | GO:0009753 | response to | 13 out of 275 | 158 out of 15002 | 0.000597 | AT1G73080\|AT4G05100\|AT3G15210\|AT1G01060\|AT3G23250\|AT2G26 |
| | | jasmonic | genes, 4.7% | genes, 1.1% | | 690\|AT1G32640\|AT1G19180\|AT5G37500\|AT4G37260\|AT3G15500\|AT4 |
| | | acid | | | | G01370\|AT2G46830 |
| | | stimulus | | | | |
| | GO:0051707 | response to | 26 out of 275 | 558 out of 15002 | 0.000872 | AT1G22070\|AT1G10170\|AT4G23190\|AT5G06320\|AT4G33950\|AT1G45 |
| | | other | genes, 9.5% | genes, 3.7% | | 145\|AT2G40140\|AT2G40000\|AT3G50970\|AT4G39090\|AT2G41430\|AT5 |
| | | organism | | | | G45110\|AT3G49530\|AT5G15090\|AT5G39580\|AT1G59870\|AT5G06290\| |
| | | | | | | AT5G61890\|AT5G14740\|AT3G10920\|AT4G36010\|AT4G01370\|AT2G24 |
| | | | | | | 570\|AT3G10985\|AT1G19180\|AT1G20440 |
| | GO:0070887 | cellular | 20 out of 275 | 374 out of 15002 | 0.00127 | AT1G22070\|AT1G05680\|AT3G15210\|AT1G59870\|AT4G33950\|AT1G62 |
| | | response to | genes, 7.3% | genes, 2.5% | | 300\|AT1G32640\|AT3G16857\|AT1G78080\|AT3G10920\|AT3G15500\|AT4 |
| | | chemical | | | | G01370\|AT1G29395\|AT4G17615\|AT3G53480\|AT2G04880\|AT1G15080\| |
| | | stimulus | | | | AT5G47230\|AT1G19180\|AT1G42990 |
| | GO:0009617 | response to | 16 out of 275 | 256 out of 15002 | 0.00134 | AT1G22070\|AT2G40000\|AT5G15090\|AT1G10170\|AT4G23190\|AT5G06 |
| | | bacterium | genes, 5.8% | genes, 1.7% | | 320\|AT1G59870\|AT5G06290\|AT4G33950\|AT5G14740\|AT1G19180\|AT3 |
| | | | | | | G10920\|AT4G39090\|AT2G24570\|AT2G41430\|AT5G45110 |
| | GO:0051704 | multi- | 26 out of 275 | 589 out of 15002 | 0.00182 | AT1G22070\|AT1G10170\|AT4G23190\|AT5G06320\|AT4G33950\|AT1G45 |
| | | organism | genes, 9.5% | genes, 3.9% | | 145\|AT2G40140\|AT2G40000\|AT3G50970\|AT4G39090\|AT2G41430\|AT5 |
| | | process | | | | G45110\|AT3G49530\|AT5G15090\|AT5G39580\|AT1G59870\|AT5G06290\| |
| | | | | | | AT5G61890\|AT5G14740\|AT3G10920\|AT4G36010\|AT4G01370\|AT2G24 |
| | | | | | | 570\|AT3G10985\|AT1G19180\|AT1G20440 |
| | GO:0006952 | defense | 30 out of 275 | 747 out of 15002 | 0.00242 | AT1G22070\|AT1G10170\|AT4G23190\|AT5G06320\|AT4G33950\|AT1G45 |
| | | response | genes, 10.9% | genes, 5% | | 145\|AT2G40140\|AT2G35930\|AT1G33590\|AT2G40000\|AT2G03760\|AT3 |
| | | | | | | G50970\|AT3G52450\|AT4G39090\|AT5G45110\|AT5G64905\|AT3G49530\| |
| | | | | | | AT5G15090\|AT5G39580\|AT1G59870\|AT5G06290\|AT5G61890\|AT5G14 |
| | | | | | | 740\|AT3G10920\|AT4G01370\|AT1G42560\|AT2G24570\|AT1G73080\|AT1 |
| | | | | | | G19180\|AT1G20440 |
| | GO:0009631 | cold | 5 out of 275 | 21 out of 15002 | 0.00297 | AT5G59820\|AT1G20440\|AT1G20450\|AT1G29395\|AT3G50970 |
| | | acclimation | genes, 1.8% | genes, 0.1% | | |
| | GO:0009642 | response to | 8 out of 275 | 78 out of 15002 | 0.0051 | AT2G32120\|AT1G17870\|AT1G74310\|AT1G10170\|AT5G59820\|AT4G37 |
| | | light | genes, 2.9% | genes, 0.5% | | 270\|AT2G41430\|AT2G17840 |
| | | intensity | | | | |
| | GO:0072511 | divalent | 6 out of 275 | 40 out of 15002 | 0.00516 | AT3G13320\|AT4G37270\|AT3G63380\|AT1G59870\|AT2G04040\|AT1G27 |
| | | inorganic | genes, 2.2% | genes, 0.3% | | 770 |
| | | cation | | | | |
| | | transport | | | | |
| | GO:0080167 | response to | 10 out of 275 | 127 out of 15002 | 0.00564 | AT3G13790\|AT3G09440\|AT1G05680\|AT5G05600\|AT4G27280\|AT1G33 |
| | | karrikin | genes, 3.6% | genes, 0.8% | | 590\|AT3G52930\|AT1G61890\|AT4G37370\|AT1G78290 |
| | GO:0071310 | cellular | 17 out of 275 | 337 out of 15002 | 0.00701 | AT1G22070\|AT1G05680\|AT3G15210\|AT1G59870\|AT4G33950\|AT1G32 |
| | | response to | genes, 6.2% | genes, 2.2% | | 640\|AT3G16857\|AT1G78080\|AT3G15500\|AT4G01370\|AT4G17615\|AT3 |
| | | organic | | | | G53480\|AT2G04880\|AT1G15080\|AT5G47230\|AT1G19180\|AT1G42990 |
| | | substance | | | | |
| | GO:0009723 | response to | 10 out of 275 | 134 out of 15002 | 0.00789 | AT4G05100\|AT1G68765\|AT3G15210\|AT5G47230\|AT1G01060\|AT3G23 |
| | | ethylene | genes, 3.6% | genes, 0.9% | | 250\|AT1G78080\|AT4G37260\|AT2G46830\|AT2G25490 |
| | | stimulus | | | | |
| ClassIIA | None | | | | | |
| ClassIIB | GO:0006950 | response to | 18 out of 49 | 1943 out of 12802 | 0.03 | AT2G35980\|AT1G80820\|AT2G46140\|AT3G24550\|AT4G12720\|AT4G39 |
| | | stress | genes, 36.7% | genes, 15.2% | | 260\|AT3G06490\|AT1G73010\|AT4G37910\|AT2G39660\|AT5G37770\|AT4 |
| | | | | | | G34150\|AT4G02380\|AT1G14550\|AT5G26030\|AT2G38470\|AT5G47910\| |
| | | | | | | AT1G14540 |
| | GO:0006979 | response to | 6 out of 49 | 271 out of 12802 | 0.03 | AT1G14550\|AT5G26030\|AT4G12720\|AT4G02380\|AT1G14540\|AT5G37 |
| | | oxidative | genes, 12.2% | genes, 2.1% | | 770 |
| | | stress | | | | |
| | GO:0009266 | response to | 8 out of 49 | 388 out of 12802 | 0.03 | AT4G34150\|AT4G37910\|AT5G47910\|AT2G38470\|AT1G80820\|AT4G02 |
| | | temperature | genes, 16.3% | genes, 3% | | 380\|AT5G37770\|AT4G39260 |
| | | stimulus | | | | |
| | GO:0009409 | response to | 6 out of 49 | 264 out of 12802 | 0.03 | AT4G34150\|AT2G38470\|AT1G80820\|AT4G02380\|AT5G37770\|AT4G39 |
| | | cold | genes, 12.2% | genes, 2.1% | | 260 |
| | GO:0009620 | response to | 5 out of 49 | 159 out of 12802 | 0.03 | AT2G38470\|AT3G06490\|AT2G39660\|AT5G47910\|AT3G24550 |
| | | fungus | genes, 10.2% | genes, 1.2% | | |
| | GO:0010411 | xyloglucan | 2 out of 49 | 6 out of 12802 | 0.03 | AT4G30280\|AT4G30290 |
| | | metabolic | genes, 4.1% | genes, 0% | | |
| | | process | | | | |
| | GO:0042221 | response to | 16 out of 49 | 1763 out of 12802 | 0.03 | AT4G37910\|AT2G46140\|AT5G37770\|AT3G02880\|AT5G01540\|AT4G12 |
| | | chemical | genes, 32.7% | genes, 13.8% | | 720\|AT2G17660\|AT4G02380\|AT4G39260\|AT1G14550\|AT5G26030\|AT2 |
| | | stimulus | | | | G38470\|AT3G06490\|AT4G18880\|AT4G11360\|AT1G14540 |
| | GO:0006334 | nucleosome | 3 out of 49 | 58 out of 12802 | 0.05 | AT4G40030\|AT1G06760\|AT4G40040 |
| | | assembly | genes, 6.1% | genes, 0.5% | | |
| | GO:0034728 | nucleosome | 3 out of 49 | 58 out of 12802 | 0.05 | AT4G40030\|AT1G06760\|AT4G40040 |
| | | organization | genes, 6.1% | genes, 0.5% | | |
| | GO:0050896 | response to | 23 out of 49 | 3396 out of 12802 | 0.05 | AT2G35980\|AT1G80820\|AT2G46140\|AT3G02880\|AT3G24550\|AT5G01 |
| | | stimulus | genes, 46.9% | genes, 26.5% | | 540\|AT4G12720\|AT4G39260\|AT3G06490\|AT1G73010\|AT4G11360\|AT4 |
| | | | | | | G37910\|AT2G39660\|AT5G37770\|AT4G34150\|AT2G17660\|AT4G02380\| |
| | | | | | | AT1G14550\|AT5G26030\|AT2G38470\|AT4G18880\|AT5G47910\|AT1G14 |
| | | | | | | 540 |
| | GO:0065004 | protein- | 3 out of 49 | 60 out of 12802 | 0.05 | AT4G40030\|AT1G06760\|AT4G40040 |
| | | DNA | genes, 6.1% | genes, 0.5% | | |
| | | complex | | | | |
| | | assembly | | | | |
| | GO:0071824 | protein- | 3 out of 49 | 60 out of 12802 | 0.05 | AT4G40030\|AT1G06760\|AT4G40040 |
| | | DNA | genes, 6.1% | genes, 0.5% | | |
| | | complex | | | | |
| | | subunit | | | | |
| | | organization | | | | |
| ClassIIIA | GO:0009081 | branched | 6 out of 269 | 27 out of 12802 | 0.01 | AT1G18270\|AT1G10070\|AT5G43430\|AT1G10060\|AT1G03090\|AT2G43 |
| | | chain | genes, 2.2% | genes, 0.2% | | 400 |
| | | family | | | | |
| | | amino acid | | | | |
| | | metabolic | | | | |
| | | process | | | | |
| | GO:0009310 | amine | 7 out of 269 | 40 out of 12802 | 0.01 | AT4G33150\|AT2G43400\|AT1G08630\|AT5G43430\|AT1G03090\|AT1G65 |
| | | catabolic | genes, 2.6% | genes, 0.3% | | 840\|AT5G54080 |
| | | process | | | | |
| | GO:0016054 | organic | 9 out of 269 | 79 out of 12802 | 0.01 | AT2G43400\|AT2G33150\|AT5G43430\|AT4G33150\|AT3G51840\|AT1G08 |
| | | acid | genes, 3.3% | genes, 0.6% | | 630\|AT5G65110\|AT1G03090\|AT5G54080 |
| | | catabolic | | | | |
| | | process | | | | |
| | GO:0042221 | response to | 62 out of 269 | 1763 out of 12802 | 0.01 | AT1G08720\|AT1G08920\|AT5G66400\|AT2G40170\|AT2G22080\|AT4G13 |
| | | chemical | genes, 23% | genes, 13.8% | | 430\|AT4G37790\|AT2G34600\|AT1G54100\|AT5G37260\|AT3G51860\|AT5 |
| | | stimulus | | | | G61590\|AT5G47390\|AT5G16970\|AT2G38750\|AT4G37220\|AT5G16960\| |
| | | | | | | AT1G04410\|AT1G49670\|AT3G11410\|AT4G32320\|AT5G67450\|AT1G08 |
| | | | | | | 090\|AT5G54500\|AT5G50200\|AT1G08830\|AT3G56240\|AT1G55020\|AT4 |
| | | | | | | G33420\|AT1G20340\|AT4G27260\|AT5G59220\|AT1G28130\|AT2G19810\| |
| | | | | | | AT3G05200\|AT2G46270\|AT5G03720\|AT3G23230\|AT1G73260\|AT1G08 |
| | | | | | | 930\|AT5G39040\|AT5G44380\|AT1G18330\|AT5G13740\|AT4G30170\|AT4 |
| | | | | | | G35770\|AT1G16150\|AT1G15050\|AT2G14170\|AT1G80460\|AT5G10450\| |
| | | | | | | AT4G39070\|AT3G14050\|AT4G21440\|AT1G02860\|AT5G18170\|AT1G68 |
| | | | | | | 850\|AT4G34350\|AT2G01570\|AT3G60690\|AT5G05340\|AT1G17190 |
| | GO:0046395 | carboxylic | 9 out of 269 | 79 out of 12802 | 0.01 | AT2G43400\|AT2G33150\|AT5G43430\|AT4G33150\|AT3G51840\|AT1G08 |
| | | acid | genes, 3.3% | genes, 0.6% | | 630\|AT5G65110\|AT1G03090\|AT5G54080 |
| | | catabolic | | | | |
| | | process | | | | |
| | GO:0006552 | leucine | 3 out of 269 | 4 out of 12802 | 0.03 | AT2G43400\|AT5G43430\|AT1G03090 |
| | | catabolic | genes, 1.1% | genes, 0% | | |
| | | process | | | | |
| | GO:0006979 | response to | 16 out of 269 | 271 out of 12802 | 0.03 | AT2G19810\|AT2G22080\|AT1G73260\|AT1G08830\|AT3G56240\|AT5G16 |
| | | oxidative | genes, 5.9% | | | 970\|AT1G68850\|AT4G33420\|AT5G44380\|AT4G30170\|AT5G16960\|AT4 |
| | | stress | | genes, 2.1% | | G35770\|AT5G05340\|AT2G14170\|AT1G49670\|AT4G32320 |
| | GO:0009063 | cellular | 6 out of 269 | 38 out of 12802 | 0.03 | AT4G33150\|AT2G43400\|AT1G08630\|AT5G43430\|AT1G03090\|AT5G54 |
| | | amino acid | genes, 2.2% | genes, 0.3% | | 080 |
| | | catabolic | | | | |
| | | process | | | | |
| | GO:0009083 | branched | 3 out of 269 | 5 out of 12802 | 0.03 | AT2G43400\|AT5G43430\|AT1G03090 |
| | | chain | genes, 1.1% | genes, 0% | | |
| | | family | | | | |
| | | amino acid | | | | |
| | | catabolic | | | | |
| | | process | | | | |
| | GO:0050896 | response to | 97 out of 269 | 3396 out of 12802 | 0.03 | AT1G08920\|AT2G43400\|AT2G33150\|AT2G40170\|AT2G22080\|AT4G13 |
| | | stimulus | genes, 36.1% | genes, 26.5% | | 430\|AT4G37790\|AT1G54100\|AT1G02670\|AT5G61590\|AT5G47390\|AT3 |
| | | | | | | G54960\|AT2G38750\|AT4G37220\|AT5G16960\|AT1G04410\|AT1G49670\| |
| | | | | | | AT3G11410\|AT4G32320\|AT1G08090\|AT5G54500\|AT1G08830\|AT1G25 |
| | | | | | | 275\|AT3G15950\|AT4G33420\|AT4G27260\|AT5G59220\|AT1G28130\|AT5 |
| | | | | | | G24470\|AT2G46270\|AT5G03720\|AT3G23230\|AT1G06520\|AT5G67320\| |
| | | | | | | AT1G73260\|AT5G39040\|AT4G30170\|AT4G35770\|AT1G16150\|AT1G31 |
| | | | | | | 480\|AT1G80460\|AT5G24530\|AT1G75800\|AT2G39980\|AT4G39070\|AT3 |
| | | | | | | G14050\|AT1G60940\|AT5G06980\|AT1G02860\|AT3G47640\|AT1G68850\| |
| | | | | | | AT2G26280\|AT5G13750\|AT3G45060\|AT1G17190\|AT5G67440\|AT5G27 |
| | | | | | | 350\|AT1G08720\|AT5G66400\|AT5G47740\|AT5G52250\|AT4G24220\|AT2 |
| | | | | | | G34600\|AT5G37260\|AT3G51860\|AT5G16970\|AT3G61060\|AT3G27690\| |
| | | | | | | AT5G67450\|AT5G47240\|AT5G50200\|AT4G01120\|AT5G61510\|AT3G56 |
| | | | | | | 240\|AT1G55020\|AT1G20340\|AT5G04770\|AT2G19810\|AT3G05200\|AT1 |
| | | | | | | G08930\|AT5G44380\|AT1G18330\|AT5G13740\|AT1G15050\|AT2G14170\| |
| | | | | | | AT1G13080\|AT5G10450\|AT5G20250\|AT2G32660\|AT4G21440\|AT1G75 |
| | | | | | | 230\|AT5G18170\|AT4G34350\|AT2G01570\|AT3G60690\|AT5G05340\|AT5 |
| | | | | | | G61600 |
| ClassIIIB | GO:0006952 | defense | 36 out of 234 | 683 out of 12802 | 1.43E−05 | AT2G38870\|AT3G52430\|AT3G25070\|AT4G11850\|AT4G23440\|AT1G11 |
| | | response | genes, 15.4% | genes, 5.3% | | 000\|AT1G57630\|AT1G18570\|AT5G41550\|AT5G58120\|AT2G34930\|AT3 |
| | | | | | | G05360\|AT3G11840\|AT1G11310\|AT3G11820\|AT2G26380\|AT1G74710\| |
| | | | | | | AT1G61560\|AT2G26560\|AT1G15890\|AT3G48090\|AT5G04720\|AT2G16 |
| | | | | | | 870\|AT4G39030\|AT5G44070\|AT1G56510\|AT5G22690\|AT4G11170\|AT3 |
| | | | | | | G52400\|AT3G28740\|AT2G19190\|AT1G17750\|AT1G05800\|AT3G13650\| |
| | | | | | | AT1G66090\|AT4G33300 |
| | GO:0050896 | response to | 100 out of 234 | 3396 out of 12802 | 3.02E−05 | AT4G23440\|AT3G52360\|AT4G17230\|AT4G16780\|AT5G24620\|AT4G17 |
| | | stimulus | genes, 42.7% | genes, 26.5% | | 260\|AT4G34180\|AT3G11840\|AT5G62390\|AT1G61560\|AT1G18890\|AT4 |
| | | | | | | G02200\|AT4G30080\|AT5G44070\|AT3G61850\|AT1G11210\|AT1G09940\| |
| | | | | | | AT2G01150\|AT5G51190\|AT1G13340\|AT3G44720\|AT2G17040\|AT1G55 |
| | | | | | | 920\|AT1G20510\|AT3G61900\|AT4G33300\|AT3G45640\|AT2G38870\|AT3 |
| | | | | | | G25070\|AT1G57630\|AT1G07520\|AT2G34930\|AT3G17020\|AT3G50480\| |
| | | | | | | AT5G62680\|AT1G80530\|AT5G61210\|AT5G44610\|AT5G66070\|AT2G26 |
| | | | | | | 560\|AT3G07390\|AT2G40180\|AT1G56510\|AT5G63770\|AT4G11170\|AT2 |
| | | | | | | G41380\|AT5G25190\|AT5G65020\|AT3G13650\|AT2G06050\|AT3G52430\| |
| | | | | | | AT1G11000\|AT5G06720\|AT5G66880\|AT3G59900\|AT5G48540\|AT1G18 |
| | | | | | | 570\|AT2G04160\|AT3G05360\|AT1G72060\|AT1G11310\|AT1G15890\|AT3 |
| | | | | | | G48090\|AT5G04720\|AT4G26120\|AT4G39030\|AT1G52560\|AT1G05710\| |
| | | | | | | AT5G24540\|AT5G22690\|AT3G52400\|AT1G05055\|AT3G28740\|AT2G19 |
| | | | | | | 190\|AT1G52200\|AT1G17750\|AT1G74430\|AT1G05800\|AT1G66090\|AT3 |
| | | | | | | G17700\|AT1G30040\|AT4G14630\|AT4G11850\|AT5G09980\|AT5G41550\| |
| | | | | | | AT5G58120\|AT3G28580\|AT1G19220\|AT3G11820\|AT2G26380\|AT1G74 |
| | | | | | | 710\|AT2G16870\|AT2G16500\|AT1G57560\|AT1G70940\|AT1G02400\|AT5 |
| | | | | | | G54170\|AT2G46590\|AT3G09270\|AT5G49620 |
| | GO:0031348 | negative | 7 out of 234 | 18 out of 12802 | 4.84E−05 | AT3G25070\|AT1G11310\|AT3G52400\|AT3G11820\|AT4G39030\|AT1G74 |
| | | regulation | genes, 3% | genes, 0.1% | | 710\|AT3G52430 |
| | | of defense | | | | |
| | | response | | | | |
| | GO:0051707 | response to | 27 out of 234 | 533 out of 12802 | 0.000515 | AT3G45640\|AT2G06050\|AT2G38870\|AT3G52430\|AT3G25070\|AT4G11 |
| | | other | genes, 11.5% | genes, 4.2% | | 850\|AT5G24620\|AT1G18570\|AT2G34930\|AT3G50480\|AT5G61210\|AT1 |
| | | organism | | | | G11310\|AT3G11820\|AT1G74710\|AT1G61560\|AT2G26560\|AT3G48090\| |
| | | | | | | AT4G39030\|AT5G44070\|AT1G56510\|AT5G24540\|AT3G52400\|AT3G28 |
| | | | | | | 740\|AT2G19190\|AT1G17750\|AT1G05800\|AT3G17700 |
| | GO:0002376 | immune | 18 out of 234 | 277 out of 12802 | 0.000657 | AT3G48090\|AT3G52430\|AT2G16870\|AT3G25070\|AT4G11850\|AT4G23 |
| | | system | genes, 7.7% | genes, 2.2% | | 440\|AT1G57630\|AT1G56510\|AT5G41550\|AT5G58120\|AT5G22690\|AT3 |
| | | process | | | | G05360\|AT3G11840\|AT1G11310\|AT1G74710\|AT1G66090\|AT1G61560\| |
| | | | | | | AT2G26560 |
| | GO:0006950 | response to | 62 out of 234 | 1943 out of 12802 | 0.000657 | AT4G23440\|AT4G17260\|AT4G34180\|AT3G11840\|AT5G62390\|AT1G61 |
| | | stress | genes, 26.5% | genes, 15.2% | | 560\|AT4G02200\|AT5G44070\|AT1G11210\|AT1G09940\|AT1G13340\|AT1 |
| | | | | | | G55920\|AT1G20510\|AT4G33300\|AT3G45640\|AT2G38870\|AT3G25070\| |
| | | | | | | AT1G57630\|AT2G34930\|AT3G17020\|AT5G44610\|AT2G26560\|AT1G56 |
| | | | | | | 510\|AT5G63770\|AT4G11170\|AT5G65020\|AT3G13650\|AT2G06050\|AT3 |
| | | | | | | G52430\|AT1G11000\|AT5G66880\|AT5G06720\|AT1G18570\|AT3G05360\| |
| | | | | | | AT1G72060\|AT1G11310\|AT1G15890\|AT3G48090\|AT5G04720\|AT4G39 |
| | | | | | | 030\|AT1G52560\|AT5G22690\|AT3G52400\|AT1G05055\|AT3G28740\|AT2 |
| | | | | | | G19190\|AT1G52200\|AT1G17750\|AT1G05800\|AT1G66090\|AT4G14630\| |
| | | | | | | AT4G11850\|AT5G41550\|AT5G58120\|AT3G11820\|AT2G26380\|AT1G74 |
| | | | | | | 710\|AT2G16870\|AT2G16500\|AT5G54170\|AT2G46590\|AT5G49620 |
| | GO:0009607 | response to | 28 out of 234 | 582 out of 12802 | 0.000657 | AT3G45640\|AT2G06050\|AT2G38870\|AT3G52430\|AT3G25070\|AT4G11 |
| | | biotic | genes, 12% | genes, 4.5% | | 850\|AT5G24620\|AT1G18570\|AT2G34930\|AT3G50480\|AT5G61210\|AT5 |
| | | stimulus | | | | G62390\|AT1G11310\|AT3G11820\|AT1G74710\|AT1G61560\|AT2G26560\| |
| | | | | | | AT3G48090\|AT4G39030\|AT5G44070\|AT1G56510\|AT5G24540\|AT3G52 |
| | | | | | | 400\|AT3G28740\|AT2G19190\|AT1G17750\|AT1G05800\|AT3G17700 |
| | GO:0051704 | multi- | 27 out of 234 | 562 out of 12802 | 0.000657 | AT3G45640\|AT2G06050\|AT2G38870\|AT3G52430\|AT3G25070\|AT4G11 |
| | | organism | genes, 11.5% | genes, 4.4% | | 850\|AT5G24620\|AT1G18570\|AT2G34930\|AT3G50480\|AT5G61210\|AT1 |
| | | process | | | | G11310\|AT3G11820\|AT1G74710\|AT1G61560\|AT2G26560\|AT3G48090\| |
| | | | | | | AT4G39030\|AT5G44070\|AT1G56510\|AT5G24540\|AT3G52400\|AT3G28 |
| | | | | | | 740\|AT2G19190\|AT1G17750\|AT1G05800\|AT3G17700 |
| | GO:0080134 | regulation | 10 out of 234 | 86 out of 12802 | 0.000674 | AT3G45640\|AT1G11310\|AT3G11820\|AT2G31880\|AT3G52430\|AT3G25 |
| | | of response | genes, 4.3% | genes, 0.7% | | 070\|AT3G52400\|AT4G39030\|AT1G74710\|AT3G05360 |
| | | to stress | | | | |
| | GO:0031347 | regulation | 9 out of 234 | 72 out of 12802 | 0.00102 | AT1G11310\|AT3G11820\|AT2G31880\|AT3G52430\|AT3G25070\|AT3G52 |
| | | of defense | genes, 3.8% | genes, 0.6% | | 400\|AT4G39030\|AT1G74710\|AT3G05360 |
| | | response | | | | |
| | GO:0045087 | innate | 16 out of 234 | 241 out of 12802 | 0.00106 | AT3G48090\|AT3G52430\|AT2G16870\|AT3G25070\|AT4G11850\|AT4G23 |
| | | immune | genes, 6.8% | genes, 1.9% | | 440\|AT1G57630\|AT1G56510\|AT5G41550\|AT5G58120\|AT5G22690\|AT1 |
| | | response | | | | G11310\|AT1G74710\|AT1G66090\|AT1G61560\|AT2G26560 |
| | GO:0006955 | immune | 16 out of 234 | 245 out of 12802 | 0.00118 | AT3G48090\|AT3G52430\|AT2G16870\|AT3G25070\|AT4G11850\|AT4G23 |
| | | response | genes, 6.8% | genes, 1.9% | | 440\|AT1G57630\|AT1G56510\|AT5G41550\|AT5G58120\|AT5G22690\|AT1 |
| | | | | | | G11310\|AT1G74710\|AT1G66090\|AT1G61560\|AT2G26560 |
| | GO:0008219 | cell death | 15 out of 234 | 221 out of 12802 | 0.00121 | AT5G22690\|AT3G48090\|AT5G04720\|AT2G16870\|AT3G25070\|AT4G23 |
| | | | genes, 6.4% | genes, 1.7% | | 440\|AT1G11000\|AT1G11310\|AT1G66090\|AT5G41550\|AT1G61560\|AT5 |
| | | | | | | G58120\|AT4G33300\|AT2G26560\|AT1G15890 |
| | GO:0016265 | death | 15 out of 234 | 221 out of 12802 | 0.00121 | AT5G22690\|AT3G48090\|AT5G04720\|AT2G16870\|AT3G25070\|AT4G23 |
| | | | genes, 6.4% | genes, 1.7% | | 440\|AT1G11000\|AT1G11310\|AT1G66090\|AT5G41550\|AT1G61560\|AT5 |
| | | | | | | G58120\|AT4G33300\|AT2G26560\|AT1G15890 |
| | GO:0016310 | phosphorylation | 33 out of 234 | 872 out of 12802 | 0.00364 | AT3G45640\|AT5G40540\|AT3G25070\|AT1G55610\|AT5G41680\|AT2G17 |
| | | | genes, 14.1% | genes, 6.8% | | 220\|AT1G51940\|AT4G09570\|AT2G31880\|AT4G28350\|AT2G19130\|AT5 |
| | | | | | | G38210\|AT1G70130\|AT3G55950\|AT2G37840\|AT3G16030\|AT1G51620\| |
| | | | | | | AT1G70530\|AT1G53430\|AT1G61370\|AT3G08760\|AT2G11520\|AT1G18 |
| | | | | | | 890\|AT4G21390\|AT5G07620\|AT1G53440\|AT1G28390\|AT5G65600\|AT1 |
| | | | | | | G04440\|AT2G39110\|AT1G17750\|AT1G53050\|AT4G39940 |
| | GO:0048583 | regulation | 12 out of 234 | 170 out of 12802 | 0.00495 | AT3G45640\|AT3G52430\|AT3G25070\|AT3G52400\|AT4G39030\|AT3G05 |
| | | of response | genes, 5.1% | genes, 1.3% | | 360\|AT5G66880\|AT4G09570\|AT1G11310\|AT3G11820\|AT2G31880\|AT1 |
| | | to stimulus | | | | G74710 |
| | GO:0006468 | protein | 32 out of 234 | 856 out of 12802 | 0.00519 | AT3G45640\|AT5G40540\|AT3G25070\|AT1G55610\|AT5G41680\|AT2G17 |
| | | phosphorylation | genes, 13.7% | genes, 6.7% | | 220\|AT1G51940\|AT4G09570\|AT2G31880\|AT4G28350\|AT2G19130\|AT5 |
| | | | | | | G38210\|AT1G70130\|AT3G55950\|AT2G37840\|AT3G16030\|AT1G51620\| |
| | | | | | | AT1G70530\|AT1G53430\|AT1G61370\|AT3G08760\|AT2G11520\|AT1G18 |
| | | | | | | 890\|AT4G21390\|AT5G07620\|AT1G53440\|AT1G28390\|AT5G65600\|AT1 |
| | | | | | | G04440\|AT2G39110\|AT1G17750\|AT1G53050 |
| | GO:0006793 | phosphorus | 34 out of 234 | 948 out of 12802 | 0.00605 | AT3G45640\|AT5G40540\|AT3G25070\|AT1G55610\|AT5G41680\|AT2G17 |
| | | metabolic | genes, 14.5% | genes, 7.4% | | 220\|AT1G51940\|AT4G09570\|AT2G31880\|AT4G28350\|AT2G19130\|AT5 |
| | | process | | | | G38210\|AT1G70130\|AT3G55950\|AT2G37840\|AT3G16030\|AT1G51620\| |
| | | | | | | AT1G70530\|AT1G53430\|AT1G61370\|AT3G08760\|AT2G11520\|AT1G18 |
| | | | | | | 890\|AT4G21390\|AT5G07620\|AT1G53440\|AT1G28390\|AT5G65600\|AT1 |
| | | | | | | G04440\|AT3G02800\|AT2G39110\|AT1G17750\|AT1G53050\|AT4G39940 |
| | GO:0006796 | phosphate | 34 out of 234 | 947 out of 12802 | 0.00605 | AT3G45640\|AT5G40540\|AT3G25070\|AT1G55610\|AT5G41680\|AT2G17 |
| | | metabolic | genes, 14.5% | genes, 7.4% | | 220\|AT1G51940\|AT4G09570\|AT2G31880\|AT4G28350\|AT2G19130\|AT5 |
| | | process | | | | G38210\|AT1G70130\|AT3G55950\|AT2G37840\|AT3G16030\|AT1G51620\| |
| | | | | | | AT1G70530\|AT1G53430\|AT1G61370\|AT3G08760\|AT2G11520\|AT1G18 |
| | | | | | | 890\|AT4G21390\|AT5G07620\|AT1G53440\|AT1G28390\|AT5G65600\|AT1 |
| | | | | | | G04440\|AT3G02800\|AT2G39110\|AT1G17750\|AT1G53050\|AT4G39940 |
| | GO:0012501 | programmed | 12 out of 234 | 185 out of 12802 | 0.00793 | AT5G22690\|AT3G48090\|AT5G04720\|AT2G16870\|AT3G25070\|AT4G23 |
| | | cell death | genes, 5.1% | genes, 1.4% | | 440\|AT1G66090\|AT5G41550\|AT5G58120\|AT4G33300\|AT2G26560\|AT1 |
| | | | | | | G15890 |
| | GO:0048585 | negative | 7 out of 234 | 62 out of 12802 | 0.00793 | AT1G11310\|AT3G11820\|AT3G52430\|AT3G25070\|AT3G52400\|AT4G39 |
| | | regulation | genes, 3% | genes, 0.5% | | 030\|AT1G74710 |
| | | of response | | | | |
| | | to stimulus | | | | |
| | GO:0010033 | response to | 36 out of 234 | 1059 out of 12802 | 0.00907 | AT3G52430\|AT4G17230\|AT5G66880\|AT3G59900\|AT4G16780\|AT1G18 |
| | | organic | genes, 15.4% | genes, 8.3% | | 570\|AT2G04160\|AT4G17260\|AT3G11840\|AT5G62390\|AT3G48090\|AT4 |
| | | substance | | | | G26120\|AT1G18890\|AT4G30080\|AT1G05710\|AT5G51190\|AT3G52400\| |
| | | | | | | AT2G17040\|AT1G17750\|AT1G74430\|AT3G61900\|AT3G45640\|AT3G25 |
| | | | | | | 070\|AT1G07520\|AT5G09980\|AT3G28580\|AT1G19220\|AT5G61210\|AT5 |
| | | | | | | G44610\|AT3G11820\|AT5G66070\|AT3G07390\|AT1G57560\|AT2G40180\| |
| | | | | | | AT5G25190\|AT5G49620 |
| | GO:0042221 | response to | 52 out of 234 | 1763 out of 12802 | 0.01 | AT2G06050\|AT3G52430\|AT4G17230\|AT5G06720\|AT5G66880\|AT3G59 |
| | | chemical | genes, 22.2% | genes, 13.8% | | 900\|AT4G16780\|AT1G18570\|AT2G04160\|AT4G17260\|AT1G72060\|AT3 |
| | | stimulus | | | | G11840\|AT5G62390\|AT3G48090\|AT4G26120\|AT1G18890\|AT4G02200\| |
| | | | | | | AT4G30080\|AT5G44070\|AT1G52560\|AT1G11210\|AT1G05710\|AT1G09 |
| | | | | | | 940\|AT5G51190\|AT3G52400\|AT1G13340\|AT1G52200\|AT2G17040\|AT1 |
| | | | | | | G17750\|AT1G74430\|AT3G61900\|AT3G45640\|AT3G25070\|AT1G07520\| |
| | | | | | | AT5G09980\|AT3G28580\|AT1G19220\|AT5G61210\|AT5G44610\|AT3G11 |
| | | | | | | 820\|AT5G66070\|AT2G26560\|AT3G07390\|AT2G16500\|AT1G57560\|AT2 |
| | | | | | | G40180\|AT4G11170\|AT2G41380\|AT5G25190\|AT5G65020\|AT3G09270\| |
| | | | | | | AT5G49620 |
| | GO:0009814 | defense | 8 out of 234 | 94 out of 12802 | 0.01 | AT3G48090\|AT1G56510\|AT1G11310\|AT3G52430\|AT3G25070\|AT4G11 |
| | | response, | genes, 3.4% | genes, 0.7% | | 850\|AT1G74710\|AT1G61560 |
| | | incompatible | | | | |
| | | interaction | | | | |
| | GO:0080135 | regulation | 4 out of 234 | 17 out of 12802 | 0.01 | AT3G45640\|AT3G25070\|AT3G52400\|AT3G11820 |
| | | of cellular | genes, 1.7% | genes, 0.1% | | |
| | | response to | | | | |
| | | stress | | | | |
| | GO:0050832 | defense | 9 out of 234 | 124 out of 12802 | 0.02 | AT2G34930\|AT2G38870\|AT3G52400\|AT1G56510\|AT1G11310\|AT3G11 |
| | | response to | genes, 3.8% | genes, 1% | | 820\|AT1G05800\|AT1G74710\|AT1G61560 |
| | | fungus | | | | |
| | GO:0010363 | regulation | 3 out of 234 | 8 out of 12802 | 0.02 | AT3G25070\|AT3G52400\|AT3G11820 |
| | | of plant- | genes, 1.3% | genes, 0.1% | | |
| | | type | | | | |
| | | hypersensitive | | | | |
| | | response | | | | |
| | GO:0009620 | response to | 10 out of 234 | 159 out of 12802 | 0.02 | AT2G06050\|AT2G34930\|AT2G38870\|AT3G52400\|AT1G56510\|AT1G11 |
| | | fungus | genes, 4.3% | genes, 1.2% | | 310\|AT3G11820\|AT1G05800\|AT1G74710\|AT1G61560 |
| | GO:0006915 | apoptosis | 9 out of 234 | 134 out of 12802 | 0.03 | AT5G22690\|AT5G04720\|AT2G16870\|AT4G23440\|AT1G66090\|AT5G41 |
| | | | genes, 3.8% | genes, 1% | | 550\|AT5G58120\|AT4G33300\|AT1G15890 |
| | GO:0009863 | salicylic | 4 out of 234 | 26 out of 12802 | 0.04 | AT3G52400\|AT3G11820\|AT3G48090\|AT3G52430 |
| | | acid | genes, 1.7% | genes, 0.2% | | |
| | | mediated | | | | |
| | | signaling | | | | |
| | | pathway | | | | |
| | GO:0010200 | response to | 8 out of 234 | 116 out of 12802 | 0.04 | AT3G45640\|AT2G17040\|AT3G11840\|AT1G07520\|AT5G51190\|AT4G26 |
| | | chitin | genes, 3.4% | genes, 0.9% | | 120\|AT5G66070\|AT4G17230 |
| | GO:0051245 | negative | 2 out of 234 | 2 out of 12802 | 0.04 | AT3G11820\|AT3G52400 |
| | | regulation | genes, 0.9% | genes, 0% | | |
| | | of cellular | | | | |
| | | defense | | | | |
| | | response | | | | |
| | GO:0071446 | cellular | 4 out of 234 | 26 out of 12802 | 0.04 | AT3G52400\|AT3G11820\|AT3G48090\|AT3G52430 |
| | | response to | genes, 1.7% | genes, 0.2% | | |
| | | salicylic | | | | |
| | | acid | | | | |
| | | stimulus | | | | |
| | GO:0031408 | oxylipin | 4 out of 234 | 27 out of 12802 | 0.05 | AT2G06050\|AT1G05800\|AT2G26560\|AT1G20510 |
| | | biosynthetic | genes, 1.7% | genes, 0.2% | | |
| | | process | | | | |

TABLE 22
bZIP1 primary targets in the N-assimilation pathway.

| Gene | Pathway role | bZIP1 target class |
|---|---|---|
| At2g26690 | Nitrate Transporter | Class I |
| At5g07440 | GDH | Class IIA |
| At1g08090 | Nitrate Transporter | ClassIIIA |
| At3g45060 | Nitrate Transporter | ClassIIIA |
| At5g18170 | GDH | ClassIIIA |
| At3g16150 | Asparaginase | ClassIIIA |
| At5g11520 | ASP | ClassIIIA |
| At5g50200 | Nitrate Transporter | ClassIIIA |

Lastly, Class III transient target genes are uniquely enriched in genes that respond early and transiently to the N-signal in planta (FIG. 29C). While all three classes of bZIP1 target genes have significant intersections with N-regulated genes in planta (p-val<0.001) (Krouk et al., 2010, Genome Biology 11(12):R123; Gutierrez et al., 2008, Proc. Natl. Acad. Sci. U.S.A. 105:4939-4944; Wang et al., 2003, Plant Physiol. 132(2):556-567; Wang et al., 2004, Plant physiology 136(1):2512-2522) (FIG. 29C, “Union” of N-response genes in planta), only Class IIIA transient targets have a significant overlap with genes induced transiently or early in response to a N-signal (within 3-6 minutes) (p-val<0.001), based on fine-scale kinetic studies of N-treatments performed in planta (Krouk et al., 2010, Genome Biology 11(12):R123) (FIG. 29C; Table 23). These transient bZIP1 targets include known early N-responders, such as the transcription factors LBD38 (At3g49940) and LBD39 (At4g37540), which respond to N-signals in as early as 3-6 min (Krouk et al., 2010, Genome Biology 11(12):R123), and are involved in regulating N-uptake and assimilation genes in planta (Rubin et al., 2009, The Plant Cell 21(11):3567-3584). Additionally, Class IIIA transient targets are uniquely enriched in rapid N-responders (FIG. 29C; Table 23), identified as genes induced within 20 min after a supply of 250 uM nitrate to roots (Wang et al., 2003, Plant Physiol. 132(2):556-567), including the nitrate transporters, NRT3.1 and NRT2.1. This result further supports the notion that the Class IIIA transient bZIP1 targets are specifically relevant to a rapid N-signaling response in planta.

TABLE 23
Class IIIA bZIP1 primary targets that transiently and rapidly up-regulated by N.
A. The 15 genes that are (1) ClassIIIA, i.e. no binding but activated and (2) transiently upregulated by N (Krouk et al., 2010).

| | |
|---|---|
| At1g08090 | ACH1, ATNRT2.1, ATNRT2:1, LIN1, NRT2, NRT2.1, NRT2:1, NRT2;1AT, nitrate transporter 2:1 |
| At5g57655 | xylose isomerase family protein |
| At3g14050 | AT-RSH2, ATRSH2, RSH2, RELA/SPOT homolog 2 |
| At3g28510 | P-loop containing nucleoside triphosphate hydrolases superfamily protein |
| At1g15380 | Lactoylglutathione lyase/glyoxalase I family protein |
| At5g56870 | BGAL4, beta-galactosidase 4 |
| At1g73260 | ATKTI1, KTI1, kunitz trypsin inhibitor 1 |
| At2g43400 | ETFQO, electron-transfer flavoprotein:ubiquinone oxidoreductase |
| At1g80460 | GLI1, NHO1, Actin-like ATPase superfamily protein |
| At1g22400 | ATUGT85A1, UGT85A1, UDP-Glycosyltransferase superfamily protein |
| At4g38490 | unknown protein; Has 30201 Blast hits to 17322 proteins in 780 species: Archae - 12; Bacteria - |
| | 1396; Metazoa - 17338; Fungi - 3422; Plants - 5037; Viruses - 0; Other Eukaryotes - 2996 (source: |
| | NCBI BLink). |
| At5g65110 | ACX2, ATACX2, acyl-CoA oxidase 2 |
| At5g04310 | Pectin lyase-like superfamily protein |
| At3g16150 | N-terminal nucleophile aminohydrolases (Ntn hydrolases) superfamily protein |
| At4g13430 | ATLEUC1, IIL1, isopropyl malate isomerase large subunit 1 |

B. The 9 genes that are (1) ClassIIIA, i.e. no binding but activated and (2) rapidly (3-6 min) upregulated by N (Krouk et al., 2010).

| | |
|---|---|
| At3g49940 | LBD38, LOB domain-containing protein 38 |
| At5g10210 | CONTAINS InterPro DOMAIN/s: C2 calcium-dependent membrane targeting |
| | (InterPro:IPR000008); BEST Arabidopsis thaliana protein match is: unknown protein |
| | (TAIR:AT5G65030.1); Has 1807 Blast hits to 1807 proteins in 277 species: Archae - 0; Bacteria - 0; |
| | Metazoa - 736; Fungi - 347; Plants - 385; Viruses - 0; Other Eukaryotes - 339 (source: NCBI |
| | BLink). |
| At2g43400 | ETFQO, electron-transfer flavoprotein:ubiquinone oxidoreductase |
| At1g22400 | ATUGT85A1, UGT85A1, UDP-Glycosyltransferase superfamily protein |
| At4g38490 | unknown protein; Has 30201 Blast hits to 17322 proteins in 780 species: Archae - 12; Bacteria - |
| | 1396; Metazoa - 17338; Fungi - 3422; Plants - 5037; Viruses - 0; Other Eukaryotes - 2996 (source: |
| | NCBI BLink). |
| At4g37540 | LBD39, LOB domain-containing protein 39 |
| At5g65110 | ACX2, ATACX2, acyl-CoA oxidase 2 |
| At5g04310 | Pectin lyase-like superfamily protein |
| At4g39780 | Integrase-type DNA-binding superfamily protein |

C. The 37 genes that are (1) ClassIIIA, i.e. no binding but activated and (2) early responder (20 min) upregulated by N (Wang et al., 2003)

| | |
|---|---|
| At5g28610 | BEST Arabidopsis thaliana protein match is: glycine-rich protein (TAIR:AT5G28630.1); Has 1536 |
| | Blast hits to 1202 proteins in 136 species: Archae - 0; Bacteria - 8; Metazoa - 888; Fungi - 120; Plants - 71; |
| | Viruses - 39; Other Eukaryotes - 410 (source: NCBI BLink). |
| At5g50200 | ATNRT3.1, NRT3.1, WR3, nitrate transmembrane transporters |
| At3g11410 | AHG3, ATPP2CA, PP2CA, protein phosphatase 2CA |
| At5g46590 | anac096, NAC096, NAC domain containing protein 96 |
| At3g49940 | LBD38, LOB domain-containing protein 38 |
| At1g14340 | RNA-binding (RRM/RBD/RNP motifs) family protein |
| At3g60690 | SAUR-like auxin-responsive protein family |
| At1g71980 | Protease-associated (PA) RING/U-box zinc finger family protein |
| At5g37260 | CIR1, RVE2, Homeodomain-like superfamily protein |
| At1g23870 | ATTPS9, TPS9, TPS9, trehalose-phosphatase/synthase 9 |
| At4g18340 | Glycosyl hydrolase superfamily protein |
| At4g03510 | ATRMA1, RMA1, RING membrane-anchor 1 |
| At1g08090 | ACH1, ATNRT2.1, ATNRT2:1, LIN1, NRT2, NRT2.1, NRT2:1, NRT2;1AT, nitrate transporter 2:1 |
| At3g53150 | UGT73D1, UDP-glucosyl transferase 73D1 |
| At5g13750 | ZIFL1, zinc induced facilitator-like 1 |
| At5g67440 | NPY3, Phototropic-responsive NPH3 family protein |
| At4g36670 | Major facilitator superfamily protein |
| At5g20885 | RING/U-box superfamily protein |
| At4g32950 | Protein phosphatase 2C family protein |
| At4g32960 | unknown protein; BEST Arabidopsis thaliana protein match is: unknown protein |
| | (TAIR:AT4G32970.1); Has 106 Blast hits to 106 proteins in 39 species: Archae - 0; Bacteria - 0; |
| | Metazoa - 62; Fungi - 0; Plants - 37; Viruses - 0; Other Eukaryotes - 7 (source: NCBI BLink). |
| At5g13110 | G6PD2, glucose-6-phosphate dehydrogenase 2 |
| At1g61740 | Sulfite exporter TauE/SafE family protein |
| At4g29950 | Ypt/Rab-GAP domain of gyp1p superfamily protein |
| At5g47740 | Adenine nucleotide alpha hydrolases-like superfamily protein |
| At2g46270 | GBF3, G-box binding factor 3 |
| At5g10210 | CONTAINS InterPro DOMAIN/s: C2 calcium-dependent membrane targeting |
| | (InterPro:IPR000008); BEST Arabidopsis thaliana protein match is: unknown protein |
| | (TAIR:AT5G65030.1); Has 1807 Blast hits to 1807 proteins in 277 species: Archae - 0; Bacteria - 0; |
| | Metazoa - 736; Fungi - 347; Plants - 385; Viruses - 0; Other Eukaryotes - 339 (source: NCBI |
| | BLink). |
| At5g47560 | ATSDAT, ATTDT, TDT, tonoplast dicarboxylate transporter |
| At3g16150 | N-terminal nucleophile aminohydrolases (Ntn hydrolases) superfamily protein |
| At4g38340 | NLP3;Plant regulator RWP-RK family protein |
| At4g39780 | Integrase-type DNA-binding superfamily protein |
| At3g15650 | alpha/beta-Hydrolases superfamily protein |
| At3g24520 | AT-HSFC1, HSFC1, heat shock transcription factor C1 |
| At4g38470 | ACT-like protein tyrosine kinase family protein |
| At1g15380 | Lactoylglutathione lyase/glyoxalase I family protein |
| At4g37540 | LBD39, LOB domain-containing protein 39 |
| At1g61660 | basic helix-loop-helix (bHLH) DNA-binding superfamily protein |
| At3g05200 | ATL6, RING/U-box superfamily protein |

A Transient Mode of bZIP1 Action Invokes a “Hit-and-Run” Model for N-Signaling.

The significant enrichment of N-relevant genes in Class III targets, links the transient mode-of-action of bZIP1 with early and transient aspects of N-nutrient signaling (FIGS. 29C & D). This transient mode-of-action could allow a small number of bZIP1 molecules to initiate and catalyze a large response to an N-signal in the GRN within minutes, without having to wait for a significant buildup of the bZIP1 protein. Two unique properties of Class III “transient” targets support this hypothesis. First, pioneer TFs have been shown to facilitate and/or initiate gene expression (Ni et al., 2009, Gene Dev 23(11):1351-1363; Magnani et al., 2011, Trends Genet. 27(11):465-474). Accordingly, bZIP1 binding to the promoter of Class III transient targets should be detected at very early time-points after DEX-induced nuclear localization of the GR-bZIP1 fusion protein (e.g. within minutes). Second, cis-motif analysis of target genes of a pioneer TF in Drosophila highlighted the specific enrichment of other TF binding motifs in close proximity to the pioneer TF motif (Satija et al., 2012, Genome Res 22(4):656-665), suggesting either active recruitment or passive enabling of binding by additional TF partners. By this model, the promoters of Class III transient bZIP1 targets should show specific enrichment for binding sites of other TFs in addition to bZIP1. Indeed, we find bZIP1 shares both of these properties, as detailed below.

To experimentally determine if any of the Class III transient targets are bound by bZIP1 at very early time-points, ChIP-Seq analysis was performed on four additional time-points after the DEX-induced nuclear import of bZIP1. 41 genes were revealed from Class III transient targets that have detectable bZIP1 binding at one or more of the earlier time-points (1, 5, 30, 60 min) (FIG. 29D; Table 20), but are not bound by bZIP1 at the 5 hour time point of our original study (FIG. 29A). Crucially, these 41 transiently bound bZIP1 targets are significantly enriched in GO-terms related to the N-signal (e.g. amino acid metabolism, p<0.05). The validated bZIP1 binding site (hybrid “ACGT” motif) (Baena-Gonzalez et al., 2007, Nature 448:938; Kang et al., 2010, Molecular Plant 3:361-373; Dietrich et al., 2011, The Plant Cell 23:381-395) is enriched in the promoters of these 41 genes (E=2.7e-3), as well as in the remaining Class III transient targets (E=1e-26). These transiently bound bZIP1 targets include NLP3, a key early regulator of nitrate signaling in plants (Konishi et al., 2013, Nature Communications 4: 1617). In this study, NLP3 is bound by bZIP1 at very early time-points (1 and 5 min), but not at the later points (30 and 60 min) following TF perturbation (FIG. 29D). Similarly, the promoter of an early response gene encoding the high-affinity nitrate transporter NRT2.1 (Krouk et al., 2010, Genome Biology 11(12):R123, is bound by bZIP1 as early as 1 and 5 min after the DEX-induced nuclear import of bZIP1, but binding is weakened at 30 min and disappears at 60 min (FIG. 29D). In summary, this time-course analysis provides physical evidence that some Class III targets are indeed transiently bound to bZIP1, only at very early time-points after bZIP1 nuclear import (1-5 min). We note that such transient TF-binding is difficult to capture, unless multiple early time-points are designed for ChIP-seq study. However, the cell-based TARGET system can identify primary targets based on the outcome of TF-binding (e.g. TF-induced gene regulation), even if TF binding is highly transient (e.g. within seconds), or is never bound stably enough to be detected at any time-point.

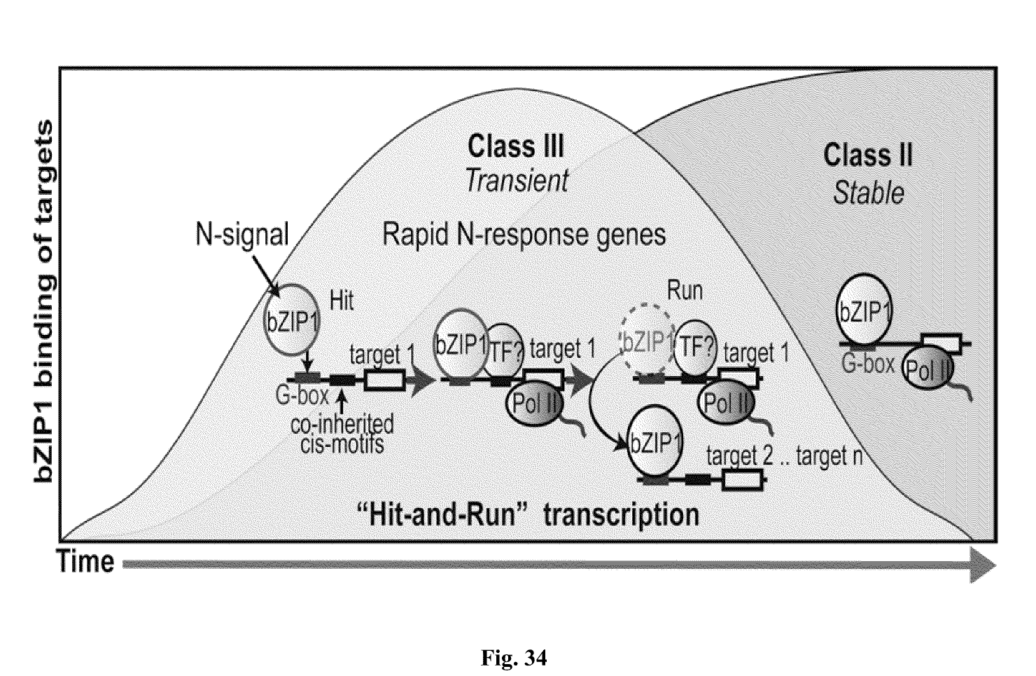

Finally, the hypothesis that bZIP1 acts as a “pioneer/catalyst” TF in N-signal propagation through a GRN, is further supported by cis-motif analysis. Specifically, the promoters of Class III “transient” bZIP1 target genes contained the largest number and most significant enrichment of cis-regulatory motifs, in addition to bZIP1-binding sites (FIG. 30). In particular, the Class IIIA transient activated genes contain the most significant enrichment of the known bZIP1 binding site (E=1.3e-52), and are specifically enriched in co-inherited cis-elements that belong to the bZIP, MYB, and GATA families (Yilmaz et al., 2011, Nucleic Acids Research 39:D1118-1122) (FIG. 30). These results support the hypothesis that bZIP1 is a pioneer TF that interacts and/or recruits other TFs, including other bZIPs and/or MYB/GATA binding factors, to temporally co-regulate target genes in response to a N-signal (FIG. 34). Indeed, bZIP1 has been reported to interact with other TFs in vitro (Ehlert et al., 2006, Plant J 46(5):890-900). (Table 24) and in vivo (Ehlert et al., 2006, Plant J 46(5):890-900; (Baena-Gonzalez et al., 2007, Nature 448:938; Kang et al., 2010, Molecular Plant 3:361-373). This list of bZIP1 interactors includes bZIP25, a gene in the Class III transient bZIP1 primary targets. In support of a collaborative relationship between bZIP1 and the GATA family TFs in mediating the N-response, one GATA TF was reported to be nitrate-inducible and involved in regulating energy metabolism, thus serving as a functional analog to bZIP1 (Bi et al., 2005, Plant Journal 44(4):680-692). Taken together, the transient binding of bZIP1 and enrichment of co-inherited binding sites for additional TFs specifically in Class III transient bZIP1 targets, supports a role for bZIP1 as a TF “pioneer/catalyst” (Satija et al., 2012, Genome Res 22(4):656-665) and a model for “hit-and-run” transcription (Schaffner, 1988, Nature 336:427-428), as depicted in FIG. 34 and discussed below.

TABLE 24
bZIP1 protein-protein interaction partners.

| | |
|---|---|
| At5g37780 | ACAM-1, CAM1, TCH1, calmodulin 1 |
| At1g66410 | ACAM-4, CAM4, calmodulin 4 |
| At5g21274 | ACAM-6, CAM6, calmodulin 6 |
| At2g41100 | ATCAL4, TCH3, Calcium-binding EF hand |
| | family protein |
| At3g51920 | ATCML9, CAM9, CML9, calmodulin 9 |
| At2g41090 | Calcium-binding EF-hand family protein |
| At3g43810 | CAM7, calmodulin 7 |
| At4g14640 | CAM8, calmodulin 8 |
| At5g41910 | MED10A, Mediator complex, subunit Med10 |
| At4g34590 | ATB2, AtbZIP11, BZIP11, GBF6, G-box binding |
| | factor 6 |
| At5g49450 | AtbZIP1, bZIP1, basic leucine-zipper 1 |
| At4g02640 | ATBZIP10, BZO2H1, bZIP transcription factor |
| | family protein |
| At2g18160 | ATBZIP2, bZIP2, GBF5, basic leucine-zipper 2 |
| At3g54620 | ATBZIP25, BZIP25, BZO2H4, basic leucine zipper 25 |
| At1g59530 | ATBZIP4, bZIP4, basic leucine-zipper 4 |
| At3g30530 | ATBZIP42, bZIP42, basic leucine-zipper 42 |
| At1g75390 | AtbZIP44, bZIP44, basic leucine-zipper 44 |
| At3g62420 | ATBZIP53, BZIP53, basic region/leucine zipper |
| | motif 53 |
| At1g13600 | AtbZIP58, bZIP58, basic leucine-zipper 58 |
| At5g28770 | AtbZIP63, BZO2H3, bZIP transcription factor |
| | family protein |
| At5g24800 | ATBZIP9, BZIP9, BZO2H2, basic leucine zipper 9 |

### 10.4. Discussion

The discovery of a large and typically overlooked class of transient primary targets of the master TF bZIP1, disclosed herein, introduces a novel perspective in the general field of dynamic GRNs. Dynamic TF-target binding studies across eukaryotes have captured many transient TF-targets (Ni et al., 2009, Gene Dev 23(11):1351-1363; Chang et al., 2013, Elife 2:e00675). However, even those fine-scale time-series ChIP studies likely miss highly temporal connections, as they require biochemically detectable TF binding in at least one time-point to identify primary TF targets. Key to the discovery of the transient targets of bZIP1 involved in rapid N-signaling, disclosed herein, is the ability to identify primary targets based on TF-induced changes in mRNA that can occur even in the absence of detectable TF binding. The cell-based system also enabled the detection of rapid and transient binding within 1 minute of TF nuclear import, owing to rapid fixation of protein-DNA complexes in plant cells lacking a cell wall. Importantly, the in planta relevance of the cell-based TARGET studies disclosed herein (FIG. 29A), confirms and complements data from bZIP1 T-DNA mutants and transgenic plants (Kang et al., 2010, Molecular Plant 3:361-373) (FIG. 29B), which are unable to distinguish primary from secondary targets, or capture transient TF-target interactions. Therefore, the transient interactions between bZIP1 and its targets uncovered in the cell-based TARGET system disclosed herein help to refine an understanding of the in planta mechanism of bZIP1.

The discovery of these transient TF targets, disclosed herein, adds a new perspective to the field of dynamic GRNs. Recent time-series studies in yeast by Lickwar et. al. reported transitive TF-target binding described as a “tread-milling” mechanism, in which a TF exhibits weak and transitive binding to some of its targets, resulting in a lower level of gene activation (Lickwar et al., 2012, Nature 484(7393):251-255). The transient bZIP1 targets detected in this study do not fit this “tread-milling” model, since there is no significant difference between the expression fold-change distributions of for Class III “transient” targets, versus Class II “stable” targets. Instead, the transient TF-target interactions uncovered herein are conceptualized to a classic, but largely forgotten, “hit-and-run” model of transcription proposed in the 1980's (Schaffner, 1988, Nature 336:427-428) (FIG. 34). This “hit-and-run” model posits that a TF can act as a trigger to organize a stable transcriptional complex, after which transcription by RNA polymerase II can continue without the TF being bound to the DNA (Schaffner, 1988, Nature 336:427-428).

In support of this “hit-and-run” transcription model, Class III “transient” targets include genes that are rapidly and transiently bound by bZIP1 at very early time-points (1-5 min) after TF nuclear import, and whose level of expression is maintained at a higher level, despite being no longer bound by bZIP1 at later time-points. Continued regulation of the bZIP1 targets (after bZIP1 is no longer bound) might be mediated by other TF partners recruited by the “trigger/pioneer” TF (FIG. 34). This model is supported by the enrichment of cis-motifs co-inherited with the known bZIP1 binding motif (Baena-Gonzalez et al., 2007, Nature 448:938; Kang et al., 2010, Molecular Plant 3:361-373; Dietrich et al., 2011, The Plant Cell 23:381-395) in the Class III transient targets (FIG. 30). This finding also supports other explanatory models for “continuous” TF networks (Biggin MD, 2011, Dev Cell 21(4):611-626; Walhout AJM, 2011, Genome Biol 12(4); Lickwar et al., 2012, Nature 484(7393):251-255), which converge on the idea that TF-binding data alone is insufficient to fully characterize regulatory networks, and that other factors (including chromatin and other TFs) may influence the action of a master TF. In this transient mode-of-action, bZIP1 can activate genes in response to a N-signal (“the hit”), while the transient nature of the TF-target association (“the run”), enables bZIP1 to act as a TF “catalyst” to rapidly induce a large set of genes needed for the N-response. In support of this “catalytic” TF model, the global targets of bZIP1 N-signaling are broad, covering 32% of the directly regulated targets of NLP7 related to the N-signal, a well-studied master regulator of the N-response (Marchive et al., 2013, Nature Communications 4). Importantly, the Class III transient bZIP1 targets play a unique role in mediating a rapid, early, and biologically relevant response to the N-signal in planta. This “hit-and-run” model, supported by our results for bZIP1, could represent a general mechanism for the deployment of an acute response to nutrient sensing, as well as other signals.

Importantly, these results have significance beyond bZIP1, N-signaling, and indeed transcend plants. Across eukaryotes, TFs are found to bind only to a small percentage of their regulated targets, as shown in plants (Monke et al., 2012, Nucleic Acids Research 40:82401; Arenhart et al., 2014, Molecular plant 7(4):709-721; Bolduc et al., 2012, Gene Dev 26(15):1685-1690), yeast (Hughes et al., 2013, Genetics 195(1):9-36) and animals (Gorski et al., 2011, Nucleic Acids Research 39:9536; Bianco et al., 2014, Cancer research 74(7):2015-2025). The large number of TF-regulated but unbound genes, including the false negatives of ChIP-seq (Chen et al., 2012, Nat Methods 9(6):609), must be dismissed as putative secondary targets in approaches that can only identify primary targets based on TF-DNA binding. Instead, it is shown herein that these typically dismissed targets, which can be identified as primary TF targets by a functional read-out in this cell-based TARGET approach (e.g. TF-induced regulation), are crucial for rapid and dynamic signal propagation, thus uncovering the “dark matter” of signal transduction that has been missed. More broadly, the approach described herein is applicable across eukaryotes, and can also be adapted to studying cell-specific GRNs, by using GFP-marked cell lines in the assay (Birnbaum K, et al., 2003, Science 302(5652):1956-1960). Moreover, this approach can identify primary targets even in cases where TF binding can never be physically detected. The transient targets thus uncovered, will reveal the elusive temporal interactions that mediate rapid and dynamic responses of GRNs to external signals.

### 11. EXAMPLE 6

As described herein, using the cell-based TARGET system, a novel class of transient TF targets that are directly regulated by the bZIP1 TF, but not detectably bound by it were identified. This class of transient targets (Class III) suggests a “hit-and-run” mode-of-action for bZIP1, where bZIP1 “hits” its target, initiates transcription, then dissociates (“run”), leaving the transcription going on even without bZIP1 binding to the promoter.

To test the hypothesis that transcription of a gene initiated by “the Hit” continues after “the Run,” an affinity-tagged UTP was used to label and capture newly synthesized mRNA. By adding this label at a time-point when the TF is not detectably bound, it can be determined whether a gene is still actively transcribed. Briefly, biosynthetic tagging of newly synthesized RNA performed using 4-thiouracil and uracil phosphoribosyltransferase (referred to as “4sU tagging” hereinafter) (Sidaway-Lee et al., 2014, Genome Biology 15 (3): R45; Zeiner et al., 2008, Methods in Molecular Biology 419: 135-46), was adapted for the cell based TARGET system in plants (Bargmann et al., 2013, Molecular Plant 6(3):978). Technically, 4sU is fed to plant protoplasts and incorporated into newly synthesized RNA. After that, total RNA is extracted from the protoplasts, and the newly synthesized RNA that is tagged with 4sU is isolated from the total RNA through biotinylation and Streptavidin magnetic beads. Next, the RNA is purified and used for transcriptomics profiling. The 4sU tagged RNA represents only the newly transcribed genes.

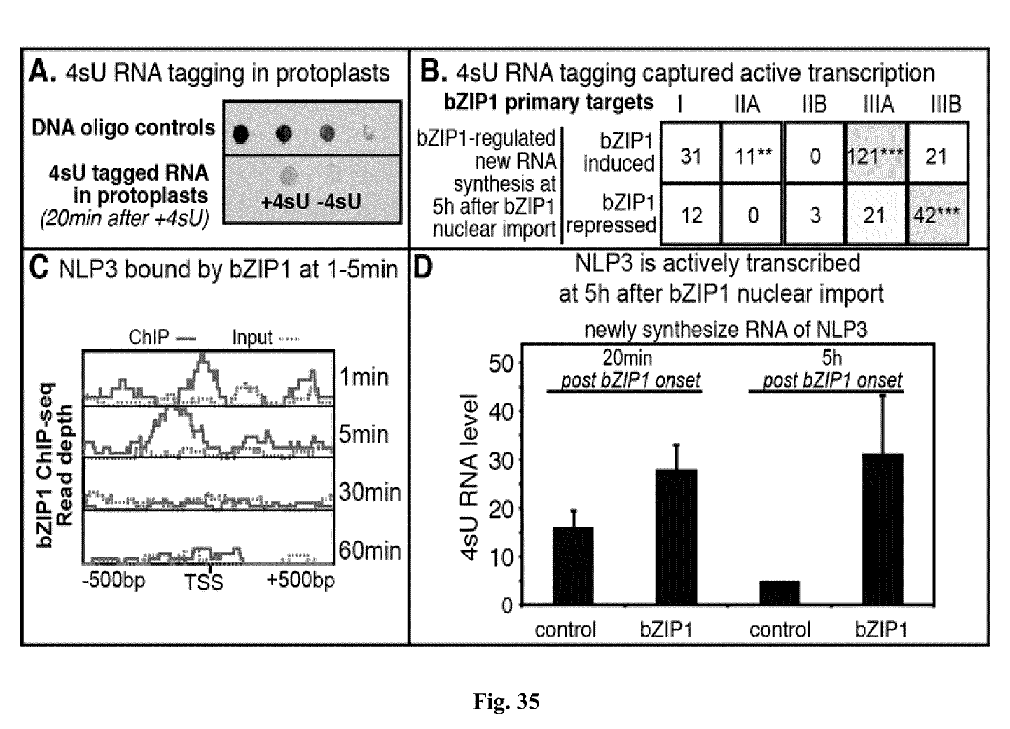

4sU tagged RNA can be detected as early as in 20 min after feeding 4sU to isolated protoplasts (FIG. 35). Using this technique, it was shown here that Class III “transient” genes have incorporated UTP label. These transient bZIP1 target genes that are activated (Class IIIA: 121 genes) or repressed (Class IIIB 42 genes). These genes are actively transcribed by bZIP1, even when bZIP1 is not bound to these targets (FIG. 29B; Table 25). These bZIP1 transient targets include the NIN-like protein 3 (NLP3; At4g38340), bound by bZIP1 at 1-5 min after the nuclear import of bZIP1 (FIG. 35C), but no longer bound by bZIP1 at 20 min, 1 hr, or 5 hr after the nuclear import of bZIP1 (FIG. 35C). These 4sU RNA tagging results show that NLP3 is actively transcribed at a higher rate in the cells that express bZIP1, even when bZIP1 does not bind to the NLP3 promoter (i.e. 5 hr after the nuclear import of bZIP1) (FIG. 35). The control in FIG. 35D is empty vector. This provides evidence for the “hit-and-run” model, which posit that bZIP1 can “hit” the target genes, and dissociate (“run”), while the induced transcription of target genes by bZIP1 can carry on even after the dissociation of bZIP1.

TABLE 25
Transient targets that are actively transcribed due to bZIP1 as validated by 4sU tagging.

| | |
|---|---|
| Gene ID | Tair 10 annotation |

A. bZIP1 Class IIIA transient targets that are transcribed higher (FC > 2) in the bZIP1
over-expressed cells compared to empty vector controls 5 hr after the bZIP1 nuclear import

| | |
|---|---|
| At5g06980 | unknown protein; BEST Arabidopsis thaliana protein match is: unknown protein |
| | (TAIR:AT3G12320.1); Has 30201 Blast hits to 17322 proteins in 780 species: Archae - 12; Bacteria - |
| | 1396; Metazoa - 17338; Fungi - 3422; Plants - 5037; Viruses - 0; Other Eukaryotes - 2996 (source: |
| | NCBI BLink). |
| At4g30170 | Peroxidase family protein |
| At3g27690 | LHCB2, LHCB2.3, LHCB2.4, photosystem II light harvesting complex gene 2.3 |
| At3g14780 | CONTAINS InterPro DOMAIN/s: Transposase, Ptta/En/Spm, plant (InterPro:IPR004252); BEST |
| | Arabidopsis thaliana protein match is: glucan synthase-like 4 (TAIR:AT3G14570.2); |
| | Has 315 Blast hits to 313 proteins in 50 species: Archae - 2; Bacteria - 16; Metazoa - 11; Fungi - 7; |
| | Plants - 181; Viruses - 2; Other Eukaryotes - 96 (source: NCBI BLink). |
| At1g30820 | CTP synthase family protein |
| At2g30600 | BTB/POZ domain-containing protein |
| At2g19320 | unknown protein; Has 9 Blast hits to 9 proteins in 4 species: Archae - 0; Bacteria - 0; Metazoa - 0; |
| | Fungi - 0; Plants - 9; Viruses - 0; Other Eukaryotes - 0 (source: NCBI BLink). |
| At1g04410 | Lactate/malate dehydrogenase family protein |
| At5g65110 | ACX2, ATACX2, acyl-CoA oxidase 2 |
| At4g18340 | Glycosyl hydrolase superfamily protein |
| At4g03510 | ATRMA1, RMA1, RING membrane-anchor 1 |
| At2g19800 | MIOX2, myo-inositol oxygenase 2 |
| At3g51730 | saposin B domain-containing protein |
| At1g56700 | Peptidase C15, pyroglutamyl peptidase I-like |
| At2g33150 | KAT2, PED1, PKT3, peroxisomal 3-ketoacyl-CoA thiolase 3 |
| At1g67810 | SUFE2, sulfur E2 |
| At5g67440 | NPY3, Phototropic-responsive NPH3 family protein |
| At5g16110 | unknown protein; FUNCTIONS IN: molecular_function unknown; INVOLVED IN: |
| | biological_process unknown; LOCATED IN: chloroplast; EXPRESSED IN: 24 plant structures; |
| | EXPRESSED DURING: 15 growth stages; BEST Arabidopsis thaliana protein match is: unknown |
| | protein (TAIR:AT3G02555.1); Has 133 Blast hits to 133 proteins in 18 species: Archae - 0; Bacteria - |
| | 0; Metazoa - 0; Fungi - 0; Plants - 133; Viruses - 0; Other Eukaryotes - 0 (source: NCBI BLink). |
| At1g75220 | Major facilitator superfamily protein |
| At1g30900 | BP80-3;3, VSR3;3, VSR6, VACUOLAR SORTING RECEPTOR 6 |
| At1g66890 | FUNCTIONS IN: molecular_function unknown; INVOLVED IN: biological_process unknown; |
| | LOCATED IN: chloroplast; EXPRESSED IN: 22 plant structures; EXPRESSED DURING: 13 growth |
| | stages; BEST Arabidopsis thaliana protein match is: 50S ribosomal protein-related |
| | (TAIR:AT5G16200.1); Has 36 Blast hits to 36 proteins in 7 species: Archae - 0; Bacteria - 0; Metazoa - |
| | 0; Fungi - 0; Plants - 36; Viruses - 0; Other Eukaryotes - 0 (source: NCBI BLink). |
| At3g49060 | U-box domain-containing protein kinase family protein |
| At3g16800 | Protein phosphatase 2C family protein |
| At1g61740 | Sulfite exporter TauE/SafE family protein |
| At5g13740 | ZIF1, zinc induced facilitator 1 |
| At5g43430 | ETFBETA, electron transfer flavoprotein beta |
| At4g21440 | ATM4, ATMYB102, MYB102, MYB102, MYB-like 102 |
| At1g55020 | ATLOX1, LOX1, lipoxygenase 1 |
| At5g19090 | Heavy metal transport/detoxification superfamily protein |
| At1g64010 | Serine protease inhibitor (SERPIN) family protein |
| At5g10210 | CONTAINS InterPro DOMAIN/s: C2 calcium-dependent membrane targeting (InterPro:IPR000008); |
| | BEST Arabidopsis thaliana protein match is: unknown protein (TAIR:AT5G65030.1); Has 1807 Blast |
| | hits to 1807 proteins in 277 species: Archae - 0; Bacteria - 0; Metazoa - 736; Fungi - 347; Plants - 385; |
| | Viruses - 0; Other Eukaryotes - 339 (source: NCBI BLink). |
| At1g75800 | Pathogenesis-related thaumatin superfamily protein |
| At5g07080 | HXXXD-type acyl-transferase family protein |
| At1g61810 | BGLU45, beta-glucosidase 45 |
| At1g67880 | beta-1,4-N-acetylglucosaminyltransferase family protein |
| At5g03720 | AT-HSFA3, HSFA3, heat shock transcription factor A3 |
| At2g38820 | Protein of unknown function (DUF506) |
| At1g65840 | ATPAO4, PAO4, polyamine oxidase 4 |
| At1g08630 | THAI, threonine aldolase 1 |
| At5g61600 | ERF104, ethylene response factor 104 |
| At1g76240 | Arabidopsis protein of unknown function (DUF241) |
| At1g28130 | GH3.17, Auxin-responsive GH3 family protein |
| At3g55150 | ATEXO70H1, EXO70H1, exocyst subunit exo70 family protein H1 |
| At3g16150 | N-terminal nucleophile aminohydrolases (Ntn hydrolases) superfamily protein |
| At4g38340 | Plant regulator RWP-RK family protein |
| At3g46690 | UDP-Glycosyltransferase superfamily protein |
| At2g19350 | Eukaryotic protein of unknown function (DUF872) |
| At1g10070 | ATBCAT-2, BCAT-2, branched-chain amino acid transaminase 2 |
| At3g43430 | RING/U-box superfamily protein |
| At3g14770 | Nodulin MtN3 family protein |
| At1g76990 | ACR3, ACT domain repeat 3 |
| At1g52240 | ATROPGEF11, PIRF1, ROPGEF11, RHO guanyl-nucleotide exchange factor 11 |
| At1g69570 | Dof-type zinc finger DNA-binding family protein |
| At1g13080 | CYP71B2, cytochrome P450, family 71, subfamily B, polypeptide 2 |
| At1g15060 | Uncharacterised conserved protein UCP031088, alpha/beta hydrolase |
| At2g14170 | ALDH6B2, aldehyde dehydrogenase 6B2 |
| At5g18650 | CHY-type/CTCHY-type/RING-type Zinc finger protein |
| At3g20410 | CPK9, calmodulin-domain protein kinase 9 |
| At3g01270 | Pectate lyase family protein |
| At2g10640 | transposable element gene |
| At4g35780 | ACT-like protein tyrosine kinase family protein |
| At3g06850 | BCE2, DIN3, LTA1, 2-oxoacid dehydrogenases acyltransferase family protein |
| At5g49650 | XK-2, XK2, xylulose kinase-2 |
| At4g15620 | Uncharacterised protein family (UPF0497) |
| At1g20340 | DRT112, PETE2, Cupredoxin superfamily protein |
| At1g55510 | BCDH BETA1, branched-chain alpha-keto acid decarboxylase E1 beta subunit |
| At2g39570 | ACT domain-containing protein |
| At4g10840 | Tetratricopeptide repeat (TPR)-like superfamily protein |
| At1g06520 | ATGPAT1, GPAT1, glycerol-3-phosphate acyltransferase 1 |
| At2g41190 | Transmembrane amino acid transporter family protein |
| At2g43060 | IBH1, ILI1 binding bHLH 1 |
| At4g35770 | ATSEN1, DIN1, SEN1, SEN1, Rhodanese/Cell cycle control phosphatase superfamily protein |
| At3g60690 | SAUR-like auxin-responsive protein family |
| At3g14760 | unknown protein; FUNCTIONS IN: molecular_function unknown; INVOLVED IN: |
| | biological_process unknown; LOCATED IN: endomembrane system; EXPRESSED IN: 6 plant |
| | structures; EXPRESSED DURING: LP.04 four leaves visible, LP.02 two leaves visible; Has 63 Blast |
| | hits to 63 proteins in 13 species: Archae - 0; Bacteria - 0; Metazoa - 0; Fungi - 0; Plants - 63; Viruses - |
| | 0; Other Eukaryotes - 0 (source: NCBI BLink). |
| At1g32460 | unknown protein; Has 19 Blast hits to 19 proteins in 8 species: Archae - 0; Bacteria - 0; Metazoa - 0; |
| | Fungi - 0; Plants - 19; Viruses - 0; Other Eukaryotes - 0 (source: NCBI BLink). |
| At2g35230 | IKU1, IKU1, VQ motif-containing protein |
| At1g09460 | Carbohydrate-binding X8 domain superfamily protein |
| At3g57420 | Protein of unknown function (DUF288) |
| At1g15050 | IAA34, indole-3-acetic acid inducible 34 |
| At3g61260 | Remorin family protein |
| At5g57655 | xylose isomerase family protein |
| At3g54960 | ATPDI1, ATPDIL1-3, PDI1, PDIL1-3, PDI-like 1-3 |
| At3g54620 | ATBZIP25, BZIP25, BZO2H4, basic leucine zipper 25 |
| At5g41610 | ATCHX18, CHX18, cation/H+ exchanger 18 |
| At4g33150 | LKR, LKR/SDH, SDH, lysine-ketoglutarate reductase/saccharopine dehydrogenase bifunctional |
| | enzyme |
| At1g03870 | FLA9, FASCICLIN-like arabinoogalactan 9 |
| At4g32870 | Polyketide cyclase/dehydrase and lipid transport superfamily protein |
| At5g01590 | unknown protein; FUNCTIONS IN: molecular_function unknown; INVOLVED IN: |
| | biological_process unknown; LOCATED IN: chloroplast, chloroplast envelope; EXPRESSED IN: 22 |
| | plant structures; EXPRESSED DURING: 13 growth stages; Has 60 Blast hits to 59 proteins in 31 |
| | species: Archae - 0; Bacteria - 20; Metazoa - 1; Fungi - 2; Plants - 33; Viruses - 0; Other Eukaryotes - |
| | 4 (source: NCBI BLink). |
| At4g32950 | Protein phosphatase 2C family protein |
| At4g19810 | Glycosyl hydrolase family protein with chitinase insertion domain |
| At2g38400 | AGT3, alanine:glyoxylate aminotransferase 3 |
| At3g13965 | pseudogene, hypothetical protein |
| At5g28050 | Cytidine/deoxycytidylate deaminase family protein |
| At2g39980 | HXXXD-type acyl-transferase family protein |
| At5g66030 | ATGRIP, GRIP, Golgi-localized GRIP domain-containing protein |
| At1g06560 | NOL1/NOP2/sun family protein |
| At5g20250 | DIN10, Raffinose synthase family protein |
| At1g03100 | Pentatricopeptide repeat (PPR) superfamily protein |
| At1g67480 | Galactose oxidase/kelch repeat superfamily protein |
| At5g08350 | GRAM domain-containing protein/ABA-responsive protein-related |
| At3g23230 | Integrase-type DNA-binding superfamily protein |
| At5g18850 | unknown protein; FUNCTIONS IN: molecular_function unknown; INVOLVED IN: |
| | biological_process unknown; LOCATED IN: endomembrane system; EXPRESSED IN: 23 plant |
| | structures; EXPRESSED DURING: 13 growth stages; Has 1807 Blast hits to 1807 proteins in 277 |
| | species: Archae - 0; Bacteria - 0; Metazoa - 736; Fungi - 347; Plants - 385; Viruses - 0; Other |
| | Eukaryotes - 339 (source: NCBI BLink). |
| At4g28040 | nodulin MtN21/EamA-like transporter family protein |
| At5g04040 | SDP1, Patatin-like phospholipase family protein |
| At3g30396 | transposable element gene |
| At1g66550 | ATWRKY67, WRKY67, WRKY DNA-binding protein 67 |
| At1g79700 | Integrase-type DNA-binding superfamily protein |
| At5g49360 | ATBXL1, BXL1, beta-xylosidase 1 |
| At4g38470 | ACT-like protein tyrosine kinase family protein |
| At1g15380 | Lactoylglutathione lyase/glyoxalase I family protein |
| At1g60940 | SNRK2-10, SNRK2.10, SRK2B, SNF1-related protein kinase 2.10 |
| At1g48840 | Plant protein of unknown function (DUF639) |
| At1g03090 | MCCA, methylcrotonyl-CoA carboxylase alpha chain, mitochondrial/3-methylcrotonyl-CoA |
| | carboxylase 1 (MCCA) |
| At3g19390 | Granulin repeat cysteine protease family protein |
| At1g32200 | ACT1, ATS1, phospholipid/glycerol acyltransferase family protein |
| At3g45300 | ATIVD, IVD, IVDH, isovaleryl-CoA-dehydrogenase |
| At3g22920 | Cyclophilin-like peptidyl-prolyl cis-trans isomerase family protein |
| At1g17190 | ATGSTU26, GSTU26, glutathione S-transferase tau 26 |
| At1g18270 | ketose-bisphosphate aldolase class-II family protein |
| At4g39730 | Lipase/lipooxygenase, PLAT/LH2 family protein |
| At4g14500 | Polyketide cyclase/dehydrase and lipid transport superfamily protein |

B. bZIP1 Class IIIB transient targets that are transcribed lower (FC <− 2) in the bZIP1
over-expressed cells compared to empty vector controls 5 hr after the bZIP1 nuclear import

| | |
|---|---|
| At5g13870 | EXGT-A4, XTH5, xyloglucan endotransglucosylase/hydrolase 5 |
| At2g17040 | anac036, NAC036, NAC domain containing protein 36 |
| At3g50480 | HR4, homolog of RPW8 4 |
| At5g60350 | unknown protein; Has 110 Blast hits to 97 proteins in 36 species: Archae - 0; Bacteria - 10; Metazoa - |
| | 39; Fungi - 2; Plants - 5; Viruses - 0; Other Eukaryotes - 54 (source: NCBI BLink). |
| At2g11520 | CRCK3, calmodulin-binding receptor-like cytoplasmic kinase 3 |
| At4g39840 | unknown protein; FUNCTIONS IN: molecular_function unknown; INVOLVED IN: |
| | biological_process unknown; LOCATED IN: endomembrane system; EXPRESSED IN: 23 plant |
| | structures; EXPRESSED DURING: 13 growth stages; Has 20719 Blast hits to 6096 proteins in 607 |
| | species: Archae - 22; Bacteria - 3243; Metazoa - 4364; Fungi - 2270; Plants - 237; |
| | Viruses - 128; Other Eukaryotes - 10455 (source: NCBI BLink). |
| At4g37400 | CYP81F3, cytochrome P450, family 81, subfamily F, polypeptide 3 |
| At5g56760 | ATSERAT1;1, SAT-52, SAT5, SERAT1;1, serine acetyltransferase 1;1 |
| At5g24540 | BGLU31, beta glucosidase 31 |
| At3g05490 | RALFL22, ralf-like 22 |
| At3g18250 | Putative membrane lipoprotein |
| At2g26480 | UGT76D1, UDP-glucosyl transferase 76D1 |
| At1g11000 | ATMLO4, MLO4, Seven transmembrane MLO family protein |
| At5g43520 | Cysteine/Histidine-rich C1 domain family protein |
| At4g28350 | Concanavalin A-like lectin protein kinase family protein |
| At3g59900 | ARGOS, auxin-regulated gene involved in organ size |
| At4g30080 | ARF16, auxin response factor 16 |
| At5g44610 | MAP18, PCAP2, microtubule-associated protein 18 |
| At1g24150 | ATFH4, FH4, formin homologue 4 |
| At5g41680 | Protein kinase superfamily protein |
| At3g47380 | Plant invertase/pectin methylesterase inhibitor superfamily protein |
| At5g24430 | Calcium-dependent protein kinase (CDPK) family protein |
| At4g16780 | ATHB-2, ATHB2, HAT4, HB-2, homeobox protein 2 |
| At4g33960 | unknown protein; FUNCTIONS IN: molecular_function unknown; INVOLVED IN: |
| | biological_process unknown; LOCATED IN: endomembrane system; EXPRESSED IN: 20 plant |
| | structures; EXPRESSED DURING: 10 growth stages; BEST Arabidopsis thaliana protein match is: |
| | unknown protein (TAIR:AT2G15830.1); Has 32 Blast hits to 32 proteins in 4 species: Archae - 0; |
| | Bacteria - 0; Metazoa - 0; Fungi - 0; Plants - 32; Viruses - 0; Other Eukaryotes - 0 (source: NCBI |
| | BLink). |
| At4g34320 | Protein of unknown function (DUF677) |
| At5g65600 | Concanavalin A-like lectin protein kinase family protein |
| At3g28740 | CYP81D1, Cytochrome P450 superfamily protein |
| At2g39700 | ATEXP4, ATEXPA4, ATHEXP ALPHA 1.6, EXPA4, expansin A4 |
| At3g20900 | unknown protein; Has 2 Blast hits to 2 proteins in 1 species: Archae - 0; Bacteria - 0; Metazoa - 0; |
| | Fungi - 0; Plants - 2; Viruses - 0; Other Eukaryotes - 0 (source: NCBI BLink). |
| At3g54980 | Pentatricopeptide repeat (PPR) superfamily protein |
| At1g53440 | Leucine-rich repeat transmembrane protein kinase |
| At1g35200 | 60S ribosomal protein L4/L1 (RPL4B), pseudogene, similar to 60S ribosomal protein L4 (fragment) |
| | GB:P49691 from (Arabidopsis thaliana); blastp match of 50% identity and 6.3e−17 P-value to |
| | SP\|Q9XF97\|RL4_PRUAR 60S ribosomal protein L4 (L1). (Apricot) {Prunus armeniaca} |
| At2g43000 | anac042, NAC042, NAC domain containing protein 42 |
| At4g15120 | VQ motif-containing protein |
| At3g48090 | ATEDS1, EDS1, alpha/beta-Hydrolases superfamily protein |
| At1g44100 | AAP5, amino acid permease 5 |
| At1g70530 | CRK3, cysteine-rich RLK (RECEPTOR-like protein kinase) 3 |
| At1g68150 | ATWRKY9, WRKY9, WRKY DNA-binding protein 9 |
| At3g02790 | zinc finger (C2H2 type) family protein |
| At1g53980 | Ubiquitin-like superfamily protein |
| At2g19190 | FRK1, FLG22-induced receptor-like kinase 1 |
| At3g29670 | HXXXD-type acyl-transferase family protein |

### 12. EXAMPLE 7

Transient TF-Targets Detected in Cells Help to Decipher Dynamic N-Regulatory Networks Operating in Planta.

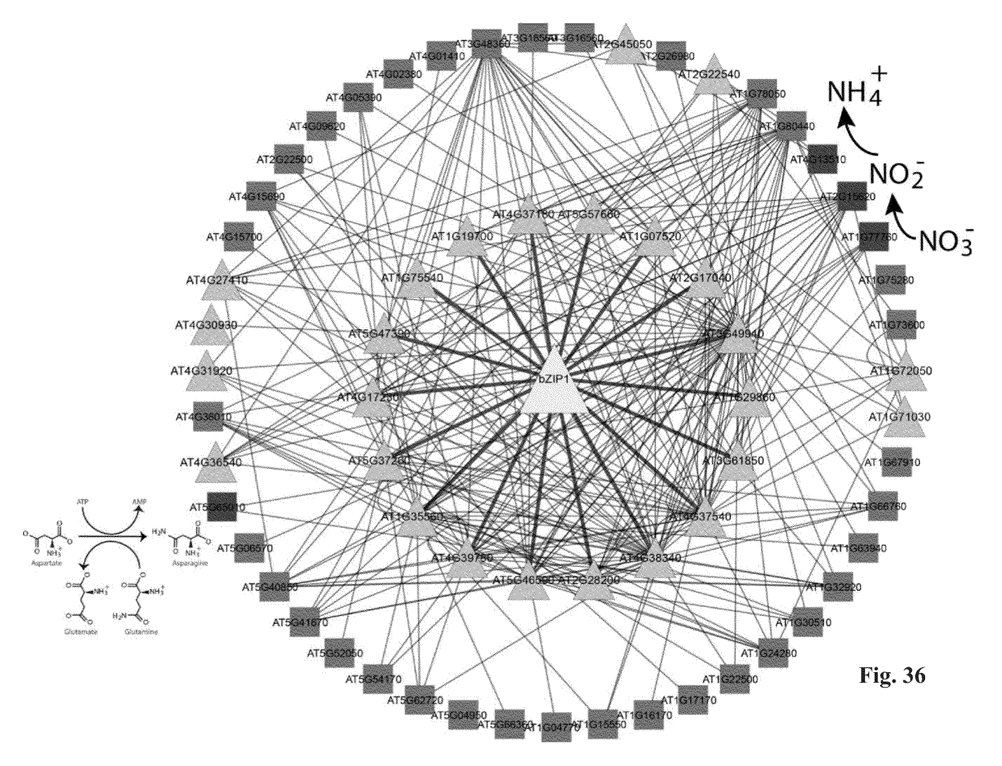

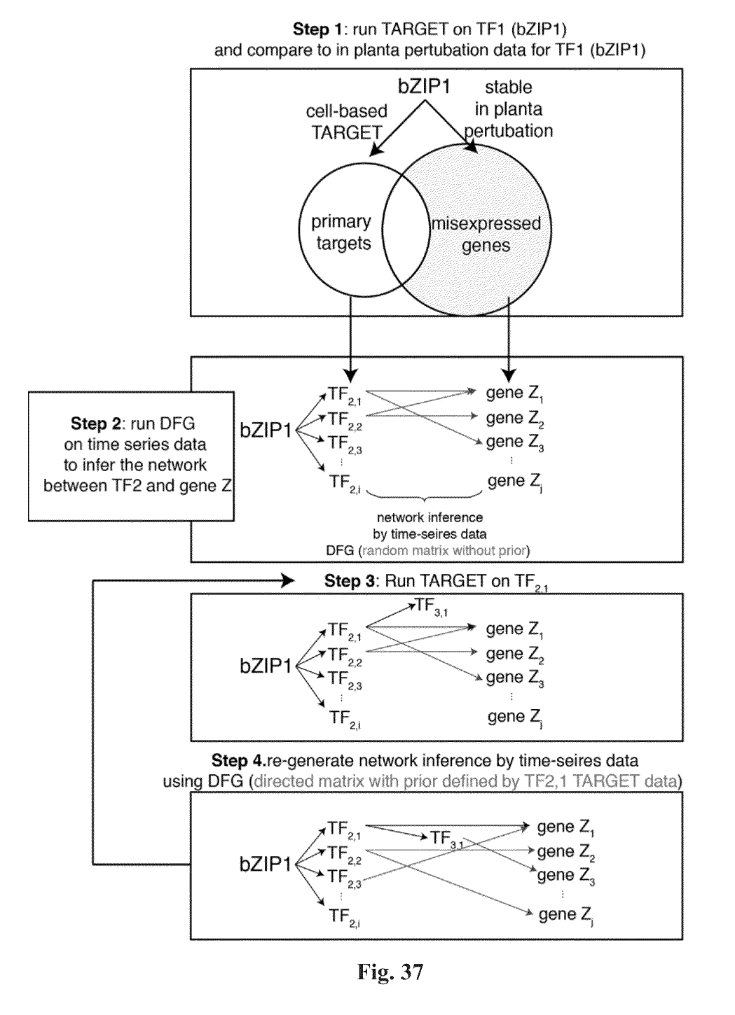

The transient TF-targets detected specifically in the TARGET cell-based system make a unique contribution to understanding how signal transduction occurs in planta. First, as the TARGET cell-based system detects only primary TF targets, this data enables the identification of direct TF-targets in the in planta TF perturbation data, which on its own cannot distinguish primary vs. secondary targets. Second, the network inference studies described herein for the proof-of-principle example bZIP1 predict that the transient bZIP1 targets (detected only in cells) are TF2's predicted to regulate secondary bZIP1 targets (detected only in planta) (FIG. 36). In FIG. 37 an approach called “Network Walking” is described to construct networks that link transient TF1→TF2 data from the TARGET cell-based system, with TF1 perturbation data in planta. The Network Walking approach uses N-response data from time-series, and Network Inference approaches including one called State-Space modeling, a form of Directed Factor Graph that was previously validated (Krouk et al., 2010, Genome Biology 11:R123; Krouk et al., 2013, Genome Biology 14(6):123). The TF2→target predictions can then be experimentally validated in the cell-based TARGET system, as described herein.

Transient TF1→T2 Targets Detected in TARGET Cell-Based System are Predicted to Regulate Secondary Targets of TF1 Identified in Planta.

The hypothesis that “transient” targets of bZIP1 detected in the cell-based TARGET system mediate N-regulation of downstream bZIP1 targets in planta was developed by the preliminary implementation of the “Network Walking” pipeline outlined in FIG. 37.

In Step 1, to identify genes potentially involved in bZIP1-mediated N-signaling in planta, bZIP1 targets identified using the cell-based TARGET system (primary targets), described herein, were combined with bZIP1 targets identified by TF perturbation in planta (primary and secondary targets) (Kang et al., 2010, Molecular Plant 3:361), and then this union of bZIP1 targets was intersected with the list of N-regulated genes from a time-course study of N-treatments performed in planta.

In Step 2, TF→target connections were inferred between the bZIP1 targets identified in the cell-based TARGET system with those identified by TF perturbation in planta, using the N-treatment time-series data and the network inference approach that was previously and validated in silico and experimentally (Directed Factor Graphs) (Krouk et al., 2010, Genome Biology 11:R123) (Step 2, FIG. 37).

The resulting network (shown in FIG. 36): The 22 TF's (depicted as triangles on the inner ring) which were identified in the cell-based TARGET system, are predicted to serve as intermediate TF2's linking bZIP1 and its downstream targets (gene Z) identified in planta (Kang et al., 2010, Molecular Plant 3:361).

Remarkably, 18/22 of these TF2's are Class III transient targets of bZIP1 detected only in the TARGET cell-based system, described herein (Inner ring of FIG. 37). As validation of their predicted role in N-signaling in planta, these transient TF2 targets of bZIP1 include TFs known to involved in N-signaling in plants (e.g. NLP3 (Konishi et al., 2013, Nature Communications 4: 1617), LBD38,39 (Rubin et al., 2009, The Plant Cell 21(11):3567-3584)). Moreover, the in planta targets of these TF2 include 7/9 N-regulated genes involved in primary assimilation of nitrate (Wang et al., 2003, Plant Physiol. 132(2):556-567). These are deemed to be secondary targets of bZIP1, as collectively they are not enriched in any of the known bZIP1 binding sites (Baena-Gonzalez et al., 2007, Nature 448:938; Kang et al., 2010, Molecular Plant 3:361; Dietrich et al., 2011, The Plant Cell 23:381-395). These lists of genes are show in Table 26.

This result supports the hypothesis that transient bZIP1 targets detected only in the TARGET cell-based system described herein, are intermediate effectors of secondary bZIP1 targets detected only in planta (Kang et al., 2010, Molecular Plant 3:361). This combined experimental and computational approach is called “Network Walking”, because it enables a “walk” from pioneer TF1→transient target (TF2)→effector target in planta (e.g. N-assimilation gene), as described below.

The General “Network Walking” Pipeline (FIG. 37):

Step 1A: Experimental: Perturb pioneer TF1 and identify symmetric difference between cell-based targets identified in TARGET (TF_{2.1-j}), and in planta targets defined by TF perturbation in planta (Z₁₋ⱼ), as well as overlap.

Step 1B: Computational: Infer edges in network. This will infer edges between potential “transient” targets detected in the cell-based TARGET system (TF_{2.1-j}) and in planta targets (Z₁₋ⱼ) of TF1 using time-series data and network inference approaches DFG (Krouk et al., 2010, Genome Biology 11:R123), Genie3 or Inferrelator (Krouk et al., 2013, Genome Biology 14(6):123).

Step 2A: Experimental: Perturb TF2 in cell-based TARGET system to validate primary TF2→gene Z edges and also identify new transient targets of TF2 (e.g. TF_{3.1-j}).

Step 2B: Computational: Rerun network inference (e.g. DFG) using time-series data from N-treated plants, this time using a directed matrix that starts with priors defined experimentally by TF2 target data (Step 3).

Outcome: This combined computational/experimental pipeline will result in a validated “Network Walk” from pioneer TF1→transient TF2.1 (identified in TARGET)→target gene Z's in planta. Another outcome will be new transient TF2→TF_{3i-j's} which may drive a new round of TF perturbation e.g. Step 3A, in a true systems biology cycle. Each iterative cycle of TF perturbation and network modeling, will build a new set of edges in the network out from the original TF1. The networks generated in Aim 2A will test the general hypothesis that transient targets detected only in the rapid and temporal cell based system, reveal “hidden steps” that mediate downstream responses in planta—but cannot be detected in planta. Thus, rather than merely using the in planta data to confirm TF-targets identified in the TARGET cell-based system, these network connections show that the transient targets identified in the cell-based TARGET system add to and refine our understanding of how dynamic networks operate in vivo, but whose specific connections elude detection in planta.

TABLE 26
Genes in bZIP1 network
>bZIP1_innerRing (Transient targets of bZIP1 only identified in the TARGET cell-based system)

| | | |
|---|---|---|
| At4g37180 | Homeodomain-like superfamily protein | |
| At4g17230 | SCL13, SCARECROW-like 13 | |
| At5g46590 | anac096, NAC096, NAC domain containing protein 96 | |
| At3g49940 | LBD38, LOB domain-containing protein 38 | This transcription factor has been |
| | | associated with N-signaling in |
| | | plants (Rubin et al., 2009, The |
| | | Plant Cell 21(11): 3567-3584). |
| At2g17040 | anac036, NAC036, NAC domain containing protein 36 | |
| At5g57660 | ATCOL5, COL5, CONSTANS-like 5 | |
| At5g37260 | CIR1, RVE2, Homeodomain-like superfamily protein | |
| At5g47390 | myb-like transcription factor family protein | |
| At1g75540 | STH2, salt tolerance homolog2 | |
| At1g35560 | TCP family transcription factor | |
| At4g39780 | Integrase-type DNA-binding superfamily protein | |
| At4g38340 | Plant regulator RWP-RK family protein (NLP3) | This transcription factor NLP3 has |
| | | been associated with N-signaling |
| | | in plants (Konishi et al., 2013, |
| | | Nature Communications 4: 1617). |
| At1g19700 | BEL10, BLH10, BEL1-like homeodomain 10 | |
| At2g28200 | C2H2-type zinc finger family protein | |
| At1g29860 | ATWRKY71, WRKY71, WRKY DNA-binding protein 71 | |
| At1g07520 | GRAS family transcription factor | |
| At4g37540 | LBD39, LOB domain-containing protein 39 | This transcription factor has been |
| | | associated with N-signaling in |
| | | plants (Rubin et al., 2009, The |
| | | Plant Cell 21(11): 3567-3584). |
| At3g61850 | DAG1, Dof-type zinc finger DNA-binding family protein | |

>bZIP1_outerRing (bZIP1 targets only identified in planta
(secondary targets) (Kang et al., 2010, Molecular Plant 3:361)

| | | |
|---|---|---|
| At5g66360 | Ribosomal RNA adenine dimethylase | |
| | family protein | |
| At4g31920 | ARR10, RR10, response regulator 10 | |
| At3g18560 | unknown protein; BEST Arabidopsis | |
| | thaliana protein match is: unknown protein | |
| | (TAIR:AT1G49000.1); Has 95 Blast hits to | |
| | 95 proteins in 13 species: Archae - 0; | |
| | Bacteria - 0; Metazoa - 0; Fungi - 0; Plants - | |
| | 95; Viruses - 0; Other Eukaryotes - 0 | |
| | (source: NCBI BLink). | |
| At4g36010 | Pathogenesis-related thaumatin superfamily | |
| | protein | |
| At5g62720 | Integral membrane HPP family protein | |
| At1g80440 | Galactose oxidase/kelch repeat superfamily | |
| | protein | |
| At4g09620 | Mitochondrial transcription termination | |
| | factor family protein | |
| At5g04950 | ATNAS1, NAS1, nicotianamine synthase 1 | |
| At4g36540 | BEE2, BR enhanced expression 2 | |
| At1g78050 | PGM, | |
| | phosphoglycerate/bisphosphoglycerate | |
| | mutase | |
| At1g63940 | MDAR6, monodehydroascorbate reductase | |
| | 6 | |
| At2g26980 | CIPK3, SnRK3.17, CBL-interacting protein | |
| | kinase 3 | |
| At4g27410 | ANAC072, RD26, NAC (No Apical | |
| | Meristem) domain transcriptional regulator | |
| | superfamily protein | |
| At1g04770 | Tetratricopeptide repeat (TPR)-like | |
| | superfamily protein | |
| At1g32920 | unknown protein; FUNCTIONS IN: | |
| | molecular_function unknown; INVOLVED | |
| | IN: response to wounding; LOCATED IN: | |
| | endomembrane system; EXPRESSED IN: | |
| | 23 plant structures; EXPRESSED | |
| | DURING: 13 growth stages; BEST | |
| | Arabidopsis thaliana protein match is: | |
| | unknown protein (TAIR:AT1G32928.1); | |
| | Has 42 Blast hits to 42 proteins in 8 species: | |
| | Archae - 0; Bacteria - 0; Metazoa - 0; Fungi - | |
| | 0; Plants - 42; Viruses - 0; Other | |
| | Eukaryotes - 0 (source: NCBI BLink). | |
| At4g02380 | AtLEA5, SAG21, senescence-associated | |
| | gene 21 | |
| At1g72050 | TFIIIA, transcription factor IIIA | |
| At1g15550 | ATGA3OX1, GA3OX1, GA4, gibberellin | |
| | 3-oxidase 1 | |
| At4g01410 | Late embryogenesis abundant (LEA) | |
| | hydroxyproline-rich glycoprotein family | |
| At5g54170 | Polyketide cyclase/dehydrase and lipid | |
| | transport superfamily protein | |
| At1g75280 | NmrA-like negative transcriptional | |
| | regulator family protein | |
| At1g77760 | GNR1, NIA1, NR1, nitrate reductase 1 | N-regulated gene involved in N-reduction/ |
| | | assimilation (Wang et al., 2003, Plant Physiol. |
| | | 132(2): 556-567). |
| At3g48360 | ATBT2, BT2, BTB and TAZ domain | |
| | protein 2 | |
| At4g13510 | AMT1;1, ATAMT1, ATAMT1;1, | N-regulated gene involved in N-reduction/ |
| | ammonium transporter 1;1 | assimilation (Wang et al., 2003, Plant Physiol. |
| | | 132(2): 556-567). |
| At5g52050 | MATE efflux family protein | |
| At5g40850 | UPM1, urophorphyrin methylase 1 | N-regulated gene involved in N-reduction/ |
| | | assimilation (Wang et al., 2003, Plant Physiol. |
| | | 132(2): 556-567). |
| At5g06570 | alpha/beta-Hydrolases superfamily protein | |
| At4g30930 | NFD1, Ribosomal protein L21 | |
| At2g22540 | AGL22, SVP, K-box region and MADS- | |
| | box transcription factor family protein | |
| At4g15690 | Thioredoxin superfamily protein | |
| At2g15620 | ATHNIR, NIR, NIR1, nitrite reductase 1 | N-regulated gene involved in N-reduction/ |
| | | assimilation (Wang et al., 2003, Plant Physiol. |
| | | 132(2): 556-567). |
| At1g30510 | ATRFNR2, RFNR2, root FNR 2 | N-regulated gene involved in N-reduction/ |
| | | assimilation (Wang et al., 2003, Plant Physiol. |
| | | 132(2): 556-567). |
| At1g66760 | MATE efflux family protein | |
| At4g05390 | ATRFNR1, RFNR1, root FNR 1 | N-regulated gene involved in N-reduction/ |
| | | assimilation (Wang et al., 2003, Plant Physiol. |
| | | 132(2): 556-567). |
| At1g17170 | ATGSTU24, GST, GSTU24, glutathione S- | |
| | transferase TAU 24 | |
| At1g67910 | unknown protein; FUNCTIONS IN: | |
| | molecular_function unknown; INVOLVED | |
| | IN: biological_process unknown; | |
| | LOCATED IN: chloroplast; EXPRESSED | |
| | IN: 21 plant structures; EXPRESSED | |
| | DURING: 12 growth stages; BEST | |
| | Arabidopsis thaliana protein match is: | |
| | unknown protein (TAIR:AT1G24577.1); | |
| | Has 167 Blast hits to 167 proteins in 19 | |
| | species: Archae - 0; Bacteria - 0; Metazoa - | |
| | 0; Fungi - 0; Plants - 167; Viruses - 0; Other | |
| | Eukaryotes - 0 (source: NCBI BLink). | |
| At1g71030 | ATMYBL2, MYBL2, MYB-like 2 | |
| At1g16170 | unknown protein; FUNCTIONS IN: | |
| | molecular_function unknown; INVOLVED | |
| | IN: biological_process unknown; | |
| | LOCATED IN: cellular_component | |
| | unknown; EXPRESSED IN: 24 plant | |
| | structures; EXPRESSED DURING: 15 | |
| | growth stages; BEST Arabidopsis thaliana | |
| | protein match is: unknown protein | |
| | (TAIR:AT1G79660.1); Has 55 Blast hits to | |
| | 55 proteins in 13 species: Archae - 0; | |
| | Bacteria - 0; Metazoa - 0; Fungi - 0; Plants - | |
| | 55; Viruses - 0; Other Eukaryotes - 0 | |
| | (source: NCBI BLink). | |
| At5g41670 | 6-phosphogluconate dehydrogenase family | |
| | protein | |
| At1g22500 | RING/U-box superfamily protein | |
| At2g45050 | GATA2, GATA transcription factor 2 | |
| At5g65010 | ASN2, asparagine synthetase 2 | N-regulated gene involved in N-reduction/ |
| | | assimilation (Wang et al., 2003, Plant Physiol. |
| | | 132(2): 556-567). |
| At1g24280 | G6PD3, glucose-6-phosphate | N-regulated gene involved in N-reduction/ |
| | dehydrogenase 3 | assimilation (Wang et al., 2003, Plant Physiol. |
| | | 132(2): 556-567). |
| At2g22500 | ATPUMP5, DIC1, UCP5, uncoupling | |
| | protein 5 | |
| At3g16560 | Protein phosphatase 2C family protein | |
| At1g73600 | S-adenosyl-L-methionine-dependent | |
| | methyltransferases superfamily protein | |
| At4g15700 | Thioredoxin superfamily protein | |

### 13. EQUIVALENTS

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference in their entireties.

What is claimed is:

1. A transgenic plant that ectopically expresses one or more hit-and-run transcription factor genes and exhibits a desired phenotype, wherein the said one or more genes comprises a polynucleotide that encodes At1g01060, At1g01720, At1g13300, At1g15100, At1g22070, At1g25550, At1g25560, At1g29160, At1g43160, At1g51700, At1g51950, At1g53910, At1 g66140, At1 g68670, At1 g68840, At1 g74660, At1 g74840, At1 g75390, At1 g77450, At1g80840, At2g04880, At2g20570, At2g22430, At2g22850, At2g24570, At2g25000, At2g28510, At2g28550, At2g30250, At2g33710, At2g38470, At2g46830, At3g01560, At3g04070, At3g06590, At3g20770, At3g25790, At3g46130, At3g47620, At3g51920, At3g54620, At3g60490, At3g61150, At3g61890, At3g62420, At4g17490, At4g17500, At4g24240, At4g27410, At4g31800, At4g34590, At4g36540, At4g37180, At4g37260, At4g37610, At4g37730, At5g05410, At5g06800, At5G10030, At5g13080, At5g14540, At5g24800, At5g39610, At5g44190, At5g47230, At5g48655, At5g49450, At5g49520, At5g56270, At5g60850, At5g63790, At5G65210, or At5g65640.

2. An isolated nucleic acid molecule that encodes (a) a chimeric protein comprising a transcription factor fused to a domain comprising an inducible nuclear localization signal; and (b) an independently expressed selectable marker.

3. The isolated nucleic acid molecule of claim 2, wherein the nucleic acid molecule is a DNA plasmid.

4. The isolated nucleic acid molecule of claim 2, wherein the domain comprising an inducible nuclear localization signal is glucocorticoid receptor.

5. The isolated nucleic acid molecule of claim 2, wherein the selectable marker is a fluorescent selection marker.

6. The isolated nucleic acid molecule of claim 5, wherein the fluorescent selection marker is green fluorescent protein, yellow fluorescent protein, red fluorescent protein, cyan fluorescent protein, or blue fluorescent protein.

7. The isolated nucleic acid molecule of claim 3, wherein the DNA plasmid is pBeaconRFP_GR, which comprises the nucleotide sequence of SEQ ID NO: 1.

8. A host cell comprising the isolated nucleic acid molecule of claim 2.

9. The host cell of claim 8, wherein the host cell is a plant protoplast.

10. The host cell of claim 9, wherein the plant protoplast is derived from one of the following genuses: Acorus, Aegilops, Allium, Amborella, Antirrhinum, Apium, Arabidopsis, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Persea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

11. The host cell of claim 8, wherein the host cell is transfected with the nucleic acid molecule.

12. The host cell of claim 11, wherein the host cell is transiently transfected with the nucleic acid molecule.

13. The host cell of any one of claim 8, wherein the host cell is derived from a genus that is different from the genus from which the transcription factor is derived from.

14. The host cell of claim 13, wherein the host cell is a plant protoplast derived from the genus Arabidopsis and the transcription factor is derived from the genus Zea.

15. A method for identifying target genes of a transcription factor comprising:
(i) transfecting host cells with the nucleic acid molecule of claim 2;
(ii) detecting host cells that express the selectable marker;
(iii) contacting the host cells that express the selectable marker with an agent that induces nuclear localization of the chimeric protein; and
(iv) detecting the level of mRNA expressed in the host cells,
wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor.

16. The method of claim 15, further comprising identifying direct target genes of the transcription factor comprising:
(v) contacting the host cells with cyclohexamide; and
(vi) detecting the level of mRNA expressed in the host cells; wherein an alteration in the level of the mRNA expressed in the host cells treated with cyclohexamide compared to the level of the mRNA expressed in the host cells not treated with cyclohexamdie indicates the identification of direct target genes of the transcription factor.

17. The method of claim 15, wherein the host cell is a plant protoplast.

18. The method of claim 17, wherein the plant protoplast is derived from one of the following genuses: Acorus, Aegilops, Allium, Amborella, Antirrhinum, Apium, Arabidopsis, Arachis, Beta, Betula, Brassica, Capsicum, Ceratopteris, Citrus, Cryptomeria, Cycas, Descurainia, Eschscholzia, Eucalyptus, Glycine, Gossypium, Hedyotis, Helianthus, Hordeum, Ipomoea, Lactuca, Linum, Liriodendron, Lotus, Lupinus, Lycopersicon, Medicago, Mesembryanthemum, Nicotiana, Nuphar, Pennisetum, Peryea, Phaseolus, Physcomitrella, Picea, Pinus, Poncirus, Populus, Prunus, Robinia, Rosa, Saccharum, Schedonorus, Secale, Sesamum, Solanum, Sorghum, Stevia, Thellungiella, Theobroma, Triphysaria, Triticum, Vitis, Zea, or Zinnia.

19. The method of claim 15, wherein the host cells are transiently transfected with the nucleic acid molecules.

20. The method of claim 15, wherein the agent that induces nuclear localization of the chimeric protein is dexamethasone.

21. The method of claim 15, wherein the step of detecting host cells that express the selectable marker is performed by Fluorescence Activated Cell Sorting.(FACS).

22. The method of claim 15 wherein the step of detecting the level of mRNA expressed in the host cells is performed by quantitative PCR, high throughput sequencing, or gene microarrays.

23. The method of claim 15, wherein the host cell is derived from a genus that is different from the genus from which the transcription factor is derived from.

24. The method of claim 23, wherein the host cell is a plant protoplast derived from the genus Arabidopsis and the transcription factor is derived from the genus Zea.

25. A method for identifying target genes of a transcription factor comprising:
(i) transfecting plant protoplasts with a DNA plasmid that encodes (a) a chimeric protein comprising a transcription factor fused to a glucocorticoid receptor; and (b) an independently expressed red fluorescent protein.
(ii) detecting the plant protoplasts that express the red fluorescent protein by performing Fluorescence Activated Cell Sorting.(FACS);
(iii) contacting the plant protoplasts that express the red fluorescent protein with an dexamethasone; and
(iv) detecting the level of mRNA expressed in the host cells,
wherein an alteration in the level of the mRNA expressed in the plant protoplasts that have nuclear localization of the chimeric protein compared to the level of the mRNA expressed in the plant protoplasts that do not have nuclear localization of the chimeric protein indicates the identification of target genes of the transcription factor.

26. The method of claim 25, further comprising (v) detecting said transcription factor binding to genomic DNA in the host cells.

27. A method for identifying target genes of a transcription factor comprising:
(i) transfecting host cells with the nucleic acid molecule of any one of claims 2-7;
(ii) detecting host cells that express the selectable marker;
(iii) contacting the host cells that express the selectable marker with an agent that induces nuclear localization of the chimeric protein; and
(iv) detecting the level of mRNA expressed in the host cells,
wherein an alteration in the level of the mRNA expressed in the host cells that have nuclear localization of the chimeric protein compared to the level of the mRNA expressed in the host cells that do not have nuclear localization of the chimeric protein indicates the identification of target genes of the transcription factor, and wherein the transcription factor is not ABI3.


SEQUENCE LISTING (1 sequence)

SEQ ID NO: 1 - DNA, 8507 bp, Artificial Sequence
  pBEACONRFP_GR
cgcatgcctgcaggtcactggattttggttttaggaattagaaattttattgatagaagtattttacaaatacaaatacatactaagggtttcttatatgctcaacacatgagcgaaaccctataagaaccctaattcccttatctgggaactactcacacattattctggagaaaaatagagagagatagatttgtagagagagactggtgatttttgcggactctagcatggccgcgggatatcaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgcccgggcgtgtcaataatatcactctgtacatccacaaacagacgataacggctctctcttttataggtgtaaaccttaaactgcatttcaccagtccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcacacctctttttgacatacttcgggtatacatatcagtatatattcttataccgcaaaaatcagcgcgcaaatacgcatactgttatctggcttttagtaagccggatccacgcgtttacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgccggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtgatatcactagttttttgatgaaacagaagctttttgatatttccatttgaatattttggtatctgattagtgatgatttcagctaacatctctgggaattcaatactcatggtcttatccaaaaatgtctggaagcagtaggtaaggagattctcaaccacctcatgcatggagtccagaagctttgtcagttggtaaaaccgttgccagttctgactggagttcccttcccttttgacgatggcttttcctagctctttgatataagtcattcgaatctcatcaaataactcttggctcttcagaccttccttaggaactgaggagagaagcagtaaggttttcatacagagatactcttcataggatacctgcaatctttgtaattcagaggagacaaacagcatgtgtttacattggtcatacatgcagggtagagacattctctgctcattaataatcagatcaggagcaaagcagagcaggtttccgcttgattgtctgtatgatctccaacccaaggcaaatgccatgagaaacatccatgagtactgtagcagggtcatttggtcatcgaggtgtaagtttctcaagcctagtatcgcctttgcccatttcactgctgcaatcacttgacgcccacctaacatgttgagtgtggtcataattctccatgctgaatctggaacagagctatcatatcctgcatacaacacctcgggttcaatcacctccagcagtgacaccaaggtaggggtgagctgtggtaatgctgcaggaactattgttttgttaggattttccgaagtgtcttgtgagactcctgcagtggcttgctgaatcatactagtgcggccgcctgcaggtcgactagaatagtaaattgtaatgttgtttgttgtttgttttgttgtggtattgttgtaaaaataccggagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatggcctttgattcagtaggaactactttcttagagactccaatctctattacttgccttggtttatgaagcaagccttgaatcgtccatactggaatagtacttctgatcttgagaaatatatctttctctgtgttcttgatgcagttagtcctgaatcttttgactgcatctttaaccttcttgggaaggtatttgatctcctggagattattactcgggtagatcgtcttgatgagacctgccgcgtaggcctctctaaccatctgtgggtcagcattctttctgaaattgaagaggctaatcttctcattatcggtggtgaacatggtatcgtcaccttctccgtcgaactttcttcctagatcgtagagatagagaaagtcgtccatggtgatctccggggcaaaggagatcagcttggctctagtcgaccatatgcccggggatcgatccccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcgggaattggatccggtaccgagctcgaattcttaggcgccggtggagtggcggccctcggcgcgctcgtactgttccacgatggtgtagtcctcgttgtgggaggtgatgtccagcttgatgtcggtcttgtaggcgccgggcagctgcacgggcttcttggccatgtaggtggtcttgacctcggcgtcgtagtggccgccgtccttcagcttcagcctcatcttgatctcgcccttcagggcgccgtcctcggggtacatccgctcggtggaggcctcccagcccatggtcttcttctgcattacggggccgtcggaggggaagttggtgccgcgcagcttcaccttgtagatgaactcgccgtcctgcagggaggagtcctgggtcacggtcaccacgccgccgtcctcgaagttcatcacgcgctcccacttgaagccctcggggaaggacagcttcaagtagtcggggatgtcggcggggtgcttcacgtaggccttggagccgtactggaactgaggggacaggatgtcccaggcgaagggcagggggccgcccttggtcaccttcagcttggcggtctgggtgccctcgtaggggcggccctcgccctcgccctcgatctcgaactcgtggccgttcacggagccctccatgcgcaccttgaagcgcatgaactccttgatgacgtcctcggaggaggccatcggatccttatcgtcatcgtcgtacagatcccgacccatttgctgtccaccagtcatgctagaatcgatagatctgctagagtcagcttgtcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggaccatcacatcaatccacttgctttgaagacgtggttggaacgtcttctttttccacgatgctcctcgtgggtgggggtccatctttgggaccactgtcggcagaggcatcttcaacgatggcctttcctttatcgcaatgatggcatttgtaggagccaccttccttttccactatcttcacaataaagtgacagatagctgggcaatggaatccgaggaggtttccggatattaccctttgttgaaaagtctcacatcggacctgcagaagcttgagctctcccatatgggagagctcccaacgcgttggatgcatagcttgagtattctatagtgtcacctaaatagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgatgcggtgtgaaataccgcacagatgcgtaaggagaaaataccgcatcaggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggcccgacgt